(19) United States
(12) Patent Application Publication
(10) Pub. No.: US 2026/0108546 A1
(43) Pub. Date: Apr. 23, 2026

(54) GIP RECEPTOR AGONIST COMPOUNDS

(71) Applicant: ELI LILLY AND COMPANY, Indianapolis, IN (US)

(72) Inventors: Thomas James BEAUCHAMP, Fishers, IN (US); Douglas Carl BEHENNA, San Juan Capistrano, CA (US); Maria del Pilar BUTELER, San Diego, CA (US); Rachel Katherine CHAMBERS, Indianapolis, IN (US); Jiehao CHEN, Carmel, IN (US); Zhaogen CHEN, Carmel, IN (US); Fortune Otuko DZEAGU, San Diego, CA (US); Erik James HEMBRE, Indianapolis, IN (US); Youming HUANG, Carmel, IN (US); Carina Ivonne JETTE, San Diego, CA (US); Steven Lee KUKLISH, Indianapolis, IN (US); Ethan Lindsay MAGNO, San Diego, CA (US); Maxwell Johnson MOORE, San Diego, CA (US); Paul Vincent RUCKER, Carlsbad, CA (US); Daniel Copley SCHMITT, Zionsville, IN (US); Lewis Robin VIDLER, Wallington (GB); Gaiying ZHAO, Carmel, IN (US)

(21) Appl. No.: 19/241,111

(22) Filed: Jun. 17, 2025

Related U.S. Application Data
(60) Provisional application No. 63/661,175, filed on Jun. 18, 2024.
(60) Provisional application No. 63/723,451, filed on Nov. 21, 2024.
(60) Provisional application No. 63/779,813, filed on Mar. 28, 2025.

(51) Int. Cl. A61K 31/675; A61K 31/415; A61K 31/437; A61K 31/4375; A61K 31/4439; A61K 31/444; A61K 31/4725; A61K 31/4985; A61K 31/501; A61K 31/506; A61K 31/513; A61K 31/519; A61K 31/53; C07B 59/00; C07D 231/12; C07D 401/14; C07D 403/14; C07D 471/04; C07D 487/04; C07D 519/00; C07F 9/6561

(52) U.S. Cl. CPC A61K 31/675; A61K 31/415; A61K 31/437; A61K 31/4375; A61K 31/4439; A61K 31/444; A61K 31/4725; A61K 31/4985; A61K 31/501; A61K 31/506; A61K 31/513; A61K 31/519; A61K 31/53; C07B 59/002; C07B 59/004; C07D 231/12; C07D 401/14; C07D 403/14; C07D 471/04; C07D 487/04; C07D 519/00; C07F 9/6561; C07B 2200/05

(57) ABSTRACT

The present disclosure provides compounds of the formula:

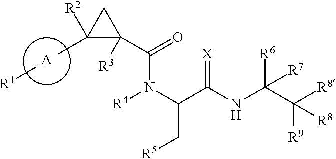

and their pharmaceutically acceptable salts, as well as pharmaceutical compositions comprising these compounds and their use in the treatment of type II diabetes mellitus and obesity.

### FIELD OF THE INVENTION

This invention relates to cyclopropane-carboxamide compounds having agonist activity at the human glucose-dependent insulinotropic polypeptide (GIP) receptor, pharmaceutically acceptable salts thereof, pharmaceutical compositions, and therapeutic uses of the compounds. The compounds may be useful in the treatment of type 2 diabetes mellitus (T2DM). Also, the compounds may be useful in the treatment of obesity.

### BACKGROUND OF THE INVENTION

Over the past several decades, the prevalence of diabetes has continued to rise. T2DM is the most common form of diabetes, accounting for approximately 90% of all diabetes. T2DM is characterized by high blood glucose levels associated mainly with insulin resistance. The current standard of care for T2DM includes diet and exercise, treatment with oral medications, and injectable glucose-lowering drugs, including incretin-based therapies such as Glucagon-like peptide-1 (GLP-1) receptor agonists. When treatment with oral medications and incretin-based therapies are insufficient, insulin treatment is considered. Despite the advances in treatment available today, many patients with T2DM are unable to reach their glycemic control goals. Uncontrolled diabetes leads to several conditions associated with increased morbidity and mortality of patients. There is a need for alternative treatments to enable more patients with T2DM to reach their glycemic treatment goals.

Obesity is a complex medical disorder resulting in excessive accumulation of adipose tissue mass. Today obesity is a global public health concern that is associated with undesired health outcomes and morbidities. Desired treatments for patients with obesity should reduce excess body weight, improve obesity-related co-morbidities, and maintain long-term weight reduction. There is a need for alternative treatment options to induce therapeutic weight loss in patients in need of such treatment.

Both GLP-1 and GIP are incretins, gastrointestinal hormones that regulate blood glucose by enhancing glucose-stimulated insulin secretion. Incretins are also involved in gastric emptying and the regulation of food intake. Incretin-based therapies have provided T2DM and obesity patients with valuable treatment options. Tirzepatide, an agonist having activity at both the GLP-1 receptor (GLP-1R) and GIPR, has been approved as an injectable for the treatment of T2DM (Mounjaro™) and for chronic weight management in adults with obesity (Zepbound™).

Injectable agents, however, have a number of drawbacks including inconvenience, pain, and the potential for injection site irritation. Therefore, patients often prefer orally administered drugs. Whilst there are a number of GLP-1R agonists in clinical development for oral administration including, for example, orforglipron (Frias et al., Lancet 2023: 402: 472-83), there are currently no such GIPR agonists.

There remains a need for alternative treatments for T2DM and obesity. In particular, there is a need for GIPR agonist compounds, especially GIPR agonist compounds which can be administered orally.

### SUMMARY OF THE INVENTION

In a first aspect, there is provided a compound of the formula:

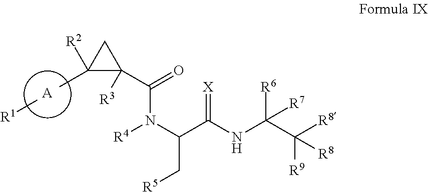

wherein
R¹ is:
   i) an 8-, 9- or 10-membered N-containing bicyclic heterocycle optionally substituted with 1 to 3 substituents independently selected from:
      C₃-C₄cycloalkyl optionally substituted with 1 or 2 halo or CH₃,
      4-membered heterocycle optionally substituted with CH₃,
      C₁-C₄alkoxy,
      C₁-C₄haloalkyl,
      C₁-C₄haloalkoxy,
      oxo,
      CN,
      halo,
      CD₃, and
      C₁-C₄alkyl optionally substituted with OCH₃, OH or C₃-C₄cycloalkyl, wherein the C₃-C₄cycloalkyl is optionally substituted with 1 or 2 halo,
   ii) NR¹⁶COR¹²,
   iii) OH, or
   iv) a 5- or 6-membered heterocycle optionally substituted with 1 or 2 substituents independently selected from: C(O)CH₃, oxo, OCH₃, CN, halo, and C₁-C₄alkyl optionally substituted with OH;
R¹² is NHR¹³, pyridyl, phenyl, OCH₃, CH₃ or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
R¹⁶ is H or CH₃;

A is phenyl, a 6-membered N-containing heteroaryl or thienyl, wherein the phenyl, heteroaryl or thienyl is optionally substituted with 1 or 2 substituents independently selected from halo, CF₃, CHF₂, CH₃, cyclopropyl, OH or CN, or when R¹ is an optionally substituted 8-, 9- or 10-membered N-containing bicyclic heterocycle A may be absent;
R² is H, CH₃, halo, CF₃ or CHF₂;
R³ is H and R⁴ is H, CH₃ or CD₃; or R³ and R⁴ together form —CH₂—CH₂—;
R⁵ is:
   i) a 8- or 9-membered N-containing bicyclic heterocycle optionally substituted with 1 or 2 substituents selected from oxo, halo and CH₃, or
   ii) a 5- or 6-membered N-containing heteroaryl optionally substituted with 1 or 2 substituents selected from CH₃, CD₃, CHF₂, OH and CH₂CN;
X is O or S;
R⁶ and R⁷ are each CH₃, or together form a cyclopropyl, cyclobutyl, oxetane, tetrahydrofuran, pyrrolidine or piperidine, wherein the cyclopropyl or cyclobutyl is optionally substituted with 1 or 2 halo and wherein the pyrrolidine or piperidine is optionally substituted with CH₃;
R⁸ is H, D, OH, CO₂R¹⁸, or

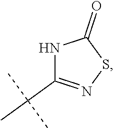

wherein R¹⁸ is H or C₁-C₄alkyl optionally substituted with OC(O)C₁-C₄alkyl or morpholine, R^{8′} is H, and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ is D, R^{8′} is D, and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo, and R^{8′} is H;
R¹⁰ is CF₃, CF₂H, halo or cyclopropyl;
R¹¹ is:
   i) a 5- or 6-membered heterocycle optionally substituted with 1 to 4 substituents independently selected from:
      halo,
      OH,
      C₁-C₄alkoxy,
      CD₃,
      C₁-C₄haloalkoxy,
      SO₂NH₂,
      CN,
      C₁-C₄haloalkyl,
      C₃-C₆cycloalkyl,
      C(O)NH₂,
      a 4- to 6-membered heterocycle optionally substituted with oxo or CH₃,
      oxo,
      CR¹⁴R¹⁵OP(O)(OH)₂,
      (CH₂)ₘP(O)(R¹⁷)₂, and
      C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 or 2 substituents independently selected from CN, CH₃, halo, oxo, C(O)O(CH₃)₃ and OH,
   iii) C₁-C₄alkoxy,
   iv) C₃-C₅cycloalkyl,
   v) halo,
   vi) (CH₂)ₙC(O)R¹⁹, wherein n is 0 or 1, R¹⁹ is OCH₃, NR¹⁴R¹⁵ or a 4- to 6-membered heterocycle,
   vii) (CH₂)ₘP(O)(R¹⁷)₂,
   viii) C₁-C₄alkyl optionally substituted with a 4- to 6-membered heterocycle or phenyl, which heterocycle or phenyl is optionally substituted by CH₃,
   ix) CN, or
   x) phenyl optionally substituted with 1 to 3 substituents independently selected from: halo, C₁-C₄alkyl, (CH₂)ₘP(O)(R¹⁷)₂, COOH and NR¹⁴R¹⁵, wherein the C₁-C₄alkyl is optionally substituted with OH; and
R¹⁴ and R¹⁵ are independently H or CH₃;
each R¹⁷ is independently C₁-C₄alkyl; and
m is 0 or 1;
or a pharmaceutically acceptable salt thereof.

In a second aspect, there is provided a pharmaceutical composition comprising a compound of Formula IX, or a pharmaceutically acceptable salt thereof, and at least one pharmaceutically acceptable carrier, diluent, or excipient.

In a third aspect, there is provided a method of treating type II diabetes mellitus in a patient comprising administering to the patient a therapeutically effective amount of a compound Formula IX, or a pharmaceutically acceptable salt thereof.

In a fourth aspect, there is provided a method of treating obesity in a patient comprising administering to the patient a therapeutically effective amount of a compound of Formula IX, or a pharmaceutically acceptable salt thereof.

In a fifth aspect, there is provided a compound of Formula IX, or a pharmaceutically acceptable salt thereof, for use in therapy.

In a sixth aspect, there is provided a compound of Formula IX, or a pharmaceutically acceptable salt thereof, for use in the treatment of type II diabetes mellitus.

In a seventh aspect, there is provided a compound Formula IX, or a pharmaceutically acceptable salt thereof, for use in treating obesity.

### DETAILED DESCRIPTION OF THE INVENTION

In an embodiment of Formula IX, there is a provided a compound of Formula IXa:

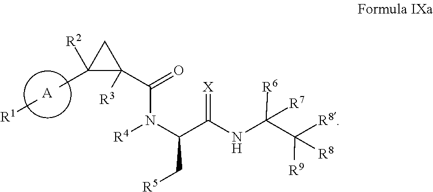

In an embodiment of Formula IX, R⁸ is H, D, OH, CO₂R¹⁸, or

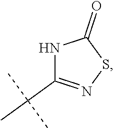

wherein R¹⁸ is H or C₁-C₄alkyl optionally substituted with OC(O)C₁-C₄alkyl or morpholine, R⁸″ is H, and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ is D, R⁸′ is D, and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo, and R⁸ is H;
R¹⁰ is CF₃, CF₂H, halo or cyclopropyl;
R¹¹ is:
   i) a 5- or 6-membered heterocycle optionally substituted with 1 to 4 substituents independently selected from:
      halo,
      OH,
      C₁-C₄alkoxy,
      CD₃,
      C₁-C₄haloalkoxy,
      SO₂NH₂,
      CN,
      C₁-C₄haloalkyl,
      C₃-C₆cycloalkyl,
      C(O)NH₂,
      a 4- to 6-membered heterocycle optionally substituted with oxo or CH₃,
      oxo,
      (CH₂)ₘP(O)(R¹⁷)₂, and
      C₁-C₄alkyl, wherein the C₁-C₄alkyl is optionally substituted with OH, OCH₃,
      C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 or 2 substituents independently selected from CN, CH₃, halo, oxo, C(O)O(CH₃)₃ and OH,
   iii) C₁-C₄alkoxy,
   iv) C₃-C₅cycloalkyl,
   v) halo,
   vi) (CH₂)ₙC(O)R¹⁹, wherein n is 0 or 1, R¹⁹ is OCH₃, NR¹⁴R¹⁵ or a 4- to 6-membered heterocycle,
   vii) (CH₂)ₘP(O)(R¹⁷)₂,
   viii) C₁-C₄alkyl optionally substituted with a 4- to 6-membered heterocycle or phenyl, which heterocycle or phenyl is optionally substituted by CH₃,
   ix) CN, or
   x) phenyl optionally substituted with 1 to 3 substituents independently selected from: halo, C₁-C₄alkyl, (CH₂)ₘP(O)(R¹⁷)₂, COOH and NR¹⁴R¹⁵, wherein the C₁-C₄alkyl is optionally substituted with OH.

In an embodiment, the compound of the present invention is a prodrug. In a particular embodiment, the prodrug is a dihydrogen phosphate (OP(O)(OH)₂) prodrug. Dihydrogen phosphate prodrugs may be formed from any aliphatic alcohol substituent. Alternatively, the dihydrogen phosphate prodrug group may be present on, for example, a 6-oxapyridazine ring.

In an embodiment of Formula IX, R⁹ is a 5- or 6-membered N-containing heteroaryl or phenyl, wherein the heteroaryl or phenyl is substituted with R¹⁰ and R¹¹, and R¹¹ is a 5- or 6-membered heterocycle substituted with CR¹⁴R¹⁵OP(O)(OH)₂ and optionally further substituted with 1 to 3 substituents independently selected from:
halo,
OH,
C₁-C₄alkoxy,
CD₃,
C₁-C₄haloalkoxy,
SO₂NH₂,
CN,
C₁-C₄haloalkyl,
C₃-C₅cycloalkyl,
C(O)NH₂,
a 4- to 6-membered heterocycle optionally substituted with oxo or CH₃,
oxo,
(CH₂)ₘP(O)(R¹⁷)₂, and
C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo.

In a particular embodiment, R¹¹ is a 5- or 6-membered N-containing heteroaryl selected from: 6-oxapyridazine, pyridine and pyrimidine, wherein the heteroaryl is substituted with CR¹⁴R¹⁵OP(O)(OH)₂.

In an embodiment, there is provided a compound of Formula V:

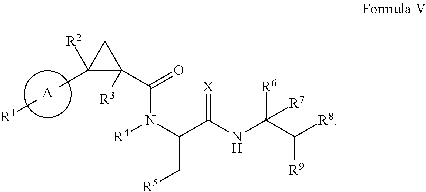

In an embodiment of Formula V, there is provided a compound wherein:
R¹ is:
   i) an 8-, 9- or 10-membered N-containing bicyclic heterocycle optionally substituted with 1 to 3 substituents independently selected from:
      C₃-C₄cycloalkyl optionally substituted with 1 or 2 halo or CH₃,
      4-membered heterocycle optionally substituted with CH₃,
      C₁-C₄alkoxy,
      C₁-C₄haloalkyl,
      oxo,
      CN,
      halo, and
      C₁-C₄alkyl optionally substituted with OCH₃, OH or C₃-C₄cycloalkyl, wherein the C₃-C₄cycloalkyl is optionally substituted with 1 or 2 halo,
   ii) NHCOR¹²,
   iii) OH, or
   iv) a 5- or 6-membered heterocycle optionally substituted with 1 or 2 substituents independently selected from: C(O)CH₃, C₁-C₄alkyl, oxo and CN;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
A is phenyl, a 6-membered N-containing heteroaryl or thienyl, wherein the phenyl, heteroaryl or thienyl is optionally substituted with 1 or 2 substituents independently selected from halo, CF₃, CHF₂, CH₃, cyclopropyl or CN, or when R¹ is an optionally substituted 8-, 9- or 10-membered N-containing bicyclic heterocycle A may be absent;
R² is H, CH₃, halo, CF₃ or CHF₂;
R³ is H and R⁴ is H, CH₃ or CD₃; or R³ and R⁴ together form —CH₂—CH₂—;
R⁵ is:
   i) a 8- or 9-membered N-containing bicyclic heterocycle optionally substituted with halo, or
   ii) a 5- or 6-membered N-containing heteroaryl optionally substituted with CH₃, CD₃, CHF₂ or OH;
X is O or S;
R⁶ and R⁷ are each CH₃ or together form a cyclopropyl;
R⁸ is H, CH₃, CO₂H or C(O)OCH₃ and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
   i) a 5- or 6-membered heterocycle optionally substituted with 1 to 4 substituents independently selected from:
      halo,
      OH,
      OCH₃,
      CD₃,
      OCHF₂,
      SO₂NH₂,
      CN,
      C₁-C₄haloalkyl,
      C₃-C₅cycloalkyl,
      C(O)NH₂,
      a 4- to 6-membered heterocycle optionally substituted with oxo,
      oxo,
      CR¹⁴R¹⁵OP(O)(OH)₂, and
      C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 or 2 substituents independently selected from CN, CH₃, halo, oxo, C(O)O(CH₃)₃ and OH,
   iii) C₁-C₄alkoxy,
   iv) C₃-C₅cycloalkyl,
   v) halo,
   vi) C(O)OCH₃, or
   vii) phenyl optionally substituted with 1 to 3 substituents independently selected from: halo and C₁-C₄alkyl, wherein the C₁-C₄alkyl is optionally substituted with OH; and
R¹⁴ and R¹⁵ are independently H or CH₃;
or a pharmaceutically acceptable salt thereof.

In an embodiment of Formula V, there is a provided a compound of Formula Va:

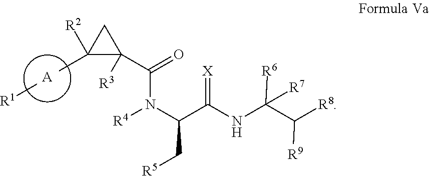

In an embodiment of any of the above formulae, R⁸ is H, CH₃, CO₂H or C(O)OCH₃ and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
   i) a 5- or 6-membered heterocycle optionally substituted with 1 to 4 substituents independently selected from:
      halo,
      OH,
      OCH₃,
      CD₃,
      OCHF₂,
      SO₂NH₂,
      CN,
      C₁-C₄haloalkyl,
      C₃-C₅cycloalkyl,
      C(O)NH₂,
      a 4- to 6-membered heterocycle optionally substituted with oxo, oxo, and
   C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 or 2 substituents independently selected from CN, CH₃, halo, oxo, C(O)O(CH₃)₃ and OH,
   iii) C₁-C₄alkoxy,
   iv) C₃-C₅cycloalkyl,
   v) halo,
   vi) C(O)OCH₃, or
   viii) phenyl optionally substituted with 1 to 3 substituents independently selected from: halo and C₁-C₄alkyl, wherein the C₁-C₄alkyl is optionally substituted with OH.

In an embodiment of any of the above formulae, R⁹ is a 5- or 6-membered N-containing heteroaryl or phenyl, wherein the heteroaryl or phenyl is substituted with R¹⁰ and R¹¹, and R¹¹ is a 5- or 6-membered heterocycle substituted with CR¹⁴R¹⁵OP(O)(OH)₂ and optionally further substituted with 1 to 3 substituents independently selected from:
halo,
OH,
OCH₃,
CD₃,
OCHF₂,
SO₂NH₂,
CN,
C₁-C₄haloalkyl,
C₃-C₅cycloalkyl,
C(O)NH₂,
a 4- to 6-membered heterocycle optionally substituted with oxo, oxo, and
C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo.

In an embodiment of any of the above formulae, R³ is H and R⁴ is H, CH₃ or CD₃. In an alternate embodiment of any of the above formulae, R³ and R⁴ together form —CH₂—CH₂—.

In an embodiment of any of the above formulae, R² is H or CH₃.

In an embodiment of Formula IX, R¹ is:
i) a 9- or 10-membered N-containing bicyclic heterocycle optionally substituted with 1 to 3 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo,
   CN
   halo, and
   C₁-C₄alkyl optionally substituted with OCH₃, OH or C₃-C₄cycloalkyl, wherein the C₃-C₄cycloalkyl is optionally substituted with 1 or 2 halo,
ii) NR¹⁶COR¹², or
iii) a 6-membered heterocycle optionally substituted with 1 or 2 substituents independently selected from: C(O)CH₃, oxo, OCH₃, halo, and C₁-C₄alkyl optionally substituted with OH;
wherein R¹² is NHR¹³, OCH₃ or C₃-C₆cycloalkyl optionally substituted with 1 or 2 halo, R¹³ is phenyl optionally substituted with 1 or 2 halo, and R¹⁶ is H or CH₃.

In an embodiment of any of the above formulae, R¹ is:
i) a 9- or 10-membered N-containing bicyclic heterocycle optionally substituted with 1 to 3 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo,
   CN,
   halo, and
   C₁-C₄alkyl optionally substituted with OCH₃, OH or C₃-C₄cycloalkyl, wherein the C₃-C₄cycloalkyl is optionally substituted with 1 or 2 halo,
ii) NHCOR¹², or
iii) a 6-membered heterocycle optionally substituted with 1 or 2 substituents independently selected from: C(O)CH₃, C₁-C₄alkyl and oxo;
wherein R¹² is NHR¹³ or C₃-C₆cycloalkyl optionally substituted with 1 or 2 halo, and R¹³ is phenyl optionally substituted with 1 or 2 halo.

In an embodiment of any of the above formulae, R¹ is:
i) a 9- or 10-membered N-containing bicyclic heterocycle optionally substituted with 1 or 2 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo, and
   C₁-C₄alkyl optionally substituted with OCH₃, OH or C₃-C₄cycloalkyl, wherein the C₃-C₄cycloalkyl is optionally substituted with 1 or 2 halo,
ii) NHCOR¹², or
iii) a 6-membered heterocycle optionally substituted with C(O)CH₃;
wherein R¹² is NHR¹³ or C₃-C₆cycloalkyl optionally substituted with 1 or 2 halo, and R¹³ is phenyl optionally substituted with 1 or 2 halo.

In an embodiment of Formula IX, A is phenyl, pyridyl, pyrimidinyl or thienyl, wherein the phenyl, pyridyl, pyrimidinyl or thienyl is optionally substituted with 1 or 2 substituents independently selected from: F, Cl, CF₃, CHF₂, CH₃, cyclopropyl and OH, or when R¹ is an optionally substituted 8-, 9- or 10-membered N-containing bicyclic heterocycle A may be absent.

In an embodiment of any of the above formulae, A is phenyl, pyridyl, pyrimidinyl or thienyl, wherein the phenyl, pyridyl, pyrimidinyl or thienyl is optionally substituted with a substituent selected from: F, Cl, CF₃, CHF₂ and CH₃, or when R¹ is an optionally substituted 8-, 9- or 10-membered N-containing bicyclic heterocycle A may be absent.

In an embodiment of any of the above formulae, A is phenyl, pyridyl, pyrimidinyl or thienyl, wherein the phenyl, pyridyl, pyrimidinyl or thienyl is optionally substituted with a substituent selected from: F, Cl, CF₃, CHF₂ and CH₃.

In an embodiment of any of the above formulae, R⁵ is:
i) a 9-membered N-containing bicyclic heterocycle, or
ii) a 5-membered N-containing heteroaryl substituted with CH₃, CD₃ or CHF₂.

In an embodiment of Formula IX, R⁶ and R⁷ are each CH₃, or together form a cyclopropyl.

In an embodiment of any of the above formulae, R⁸ is H. In an embodiment of Formula IX, R⁸ and R^{8′} are H.

In an embodiment of any of the above formulae, R⁹ is:
phenyl substituted with F, or
a 5- or 6-membered N-containing heteroaryl optionally substituted with R¹⁰ or with R¹⁰ and R¹¹.

In an embodiment of Formula IX, R¹¹ is:
phenyl substituted with F, or
a 5- or 6-membered N-containing heteroaryl optionally substituted with R¹⁰ or with R¹⁰ and R¹¹;
wherein R¹⁰ is CF₃, CF₂H or F, and R¹¹ is:
   i) a 5- or 6-membered heterocycle optionally substituted with 1 to 4 substituents independently selected from:
      halo,
      OH,
      CD₃,
      C₁-C₄haloalkoxy,
      CN,
      C₁-C₄haloalkyl,
      C₃-C₅cycloalkyl,
      C(O)NH₂,
      a 4- to 6-membered heterocycle optionally substituted with oxo,
      oxo,
      CR¹⁴R¹⁵OP(O)(OH)₂, and
      C₁-C₄alkyl, wherein said C₁-C₄ alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 to 2 substituents independently selected from CH₃, halo, oxo and C(O)O(CH₃)₃,
   iii) C₃-C₅cycloalkyl,
   iv) halo,
   v) C(O)OCH₃,
   vi) P(O)(CH₃)₂, or
   vii) phenyl optionally substituted with 1 to 3 substituents independently selected from: halo, P(O)(CH₃)₂, and C₁-C₄alkyl, wherein the C₁-C₄alkyl is optionally substituted with OH;
R¹⁴ and R¹⁵ are independently H or CH₃.

In an embodiment of any of the above formulae, R⁹ is:
a 5- or 6-membered N-containing heteroaryl optionally substituted with R¹⁰ or with R¹⁰ and R¹¹;
wherein R¹⁰ is CF₃, CF₂H or F, and R¹¹ is:
   i) a 5- or 6-membered heterocycle optionally substituted with 1 to 4 substituents independently selected from:
      halo,
      OH,
      CD₃,
      OCHF₂,
      CN,
      C₁-C₄haloalkyl,
      C₃-C₅cycloalkyl,
      C(O)NH₂,
      a 4- to 6-membered heterocycle optionally substituted with oxo,
      oxo,
      CR¹⁴R¹⁵OP(O)(OH)₂, and
      C₁-C₄alkyl, wherein said C₁-C₄ alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 to 2 substituents independently selected from CH₃, halo, oxo and C(O)O(CH₃)₃,
   iii) C₃-C₅cycloalkyl,
   iv) halo,
   v) C(O)OCH₃, or
   vi) phenyl optionally substituted with 1 to 3 substituents independently selected from: halo and C₁-C₄alkyl, wherein the C₁-C₄alkyl is optionally substituted with OH;
R¹⁴ and R¹⁵ are independently H or CH₃.

In an embodiment of any of the above formulae, R⁹ is:
a 5- or 6-membered N-containing heteroaryl optionally substituted with R¹⁰ or with R¹⁰ and R¹¹;
wherein R¹⁰ is CF₃, CF₂H or F, and R¹¹ is:
   i) a 5- or 6-membered heterocycle optionally substituted with 1 to 4 substituents independently selected from:
      halo,
      OH,
      CD₃,
      OCHF₂,
      CN,
      C₁-C₄haloalkyl,
      C₃-C₅cycloalkyl,
      C(O)NH₂,
      a 4- to 6-membered heterocycle optionally substituted with oxo,
      oxo, and
      C₁-C₄alkyl, wherein said C₁-C₄ alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 to 2 substituents independently selected from CH₃, halo, oxo and C(O)O(CH₃)₃,
   iii) C₃-C₅cycloalkyl,
   iv) halo,
   v) C(O)OCH₃, or
   vi) phenyl optionally substituted with 1 to 3 substituents independently selected from: halo and C₁-C₄alkyl, wherein the C₁-C₄alkyl is optionally substituted with OH;
R¹⁴ and R¹⁵ are independently H or CH₃.

In an embodiment, there is provided a compound of Formula VI:

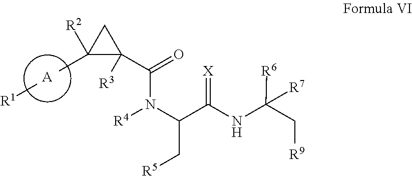

wherein
R¹ is:
   i) a 9- or 10-membered N-containing bicyclic heterocycle optionally substituted with 1 to 3 substituents independently selected from:
      C₃-C₄cycloalkyl optionally substituted with 1 or 2 halo or CH₃,
      4-membered heterocycle optionally substituted with CH₃,
      C₁-C₄alkoxy,
      C₁-C₄haloalkyl,
      oxo,
      CN,
      halo, and
      C₁-C₄alkyl optionally substituted with OCH₃, OH or C₃-C₄cycloalkyl, wherein the C₃-C₄cycloalkyl is optionally substituted with 1 or 2 halo,
   ii) NR¹⁶COR¹², or
   iii) a 6-membered heterocycle optionally substituted with 1 or 2 substituents independently selected from: C(O)CH₃, oxo, OCH₃, halo, and C₁-C₄alkyl optionally substituted with OH;
R¹² is NHR¹³, OCH₃ or C₃-C₆cycloalkyl optionally substituted with 1 or 2 halo;
R¹³ is phenyl optionally substituted with 1 or 2 halo;
R¹⁶ is H or CH₃;
A is phenyl, pyridyl, pyrimidinyl or thienyl, wherein the phenyl, pyridyl, pyrimidinyl or thienyl is optionally substituted with 1 or 2 substituents independently selected from: F, Cl, CF₃, CHF₂, CH₃, cyclopropyl and OH, or when R¹ is an optionally substituted 8-, 9- or 10-membered N-containing bicyclic heterocycle A may be absent;
R² is H or CH₃;
R³ is H and R⁴ is H, CH₃ or CD₃; or R³ and R⁴ together form —CH₂—CH₂—;
R⁵ is:
   i) a 9-membered N-containing bicyclic heterocycle, or
   ii) a 5-membered N-containing heteroaryl substituted with CH₃, CD₃ or CHF₂;
X is O or S;
R⁶ and R⁷ are each CH₃ or together form a cyclopropyl;
R⁹ is:
phenyl substituted with F, or
a 5- or 6-membered N-containing heteroaryl optionally substituted with R¹⁰ or with R¹⁰ and R¹¹;
R¹⁰ is CF₃, CF₂H or F;
R¹¹ is:
   i) a 5- or 6-membered heterocycle optionally substituted with 1 to 4 substituents independently selected from:
      halo,
      OH,
      CD₃,
      C₁-C₄haloalkoxy,
      CN,
      C₁-C₄haloalkyl,
      C₃-C₅cycloalkyl,
      C(O)NH₂,
      a 4- to 6-membered heterocycle optionally substituted with oxo,
      oxo,
      CR¹⁴R¹⁵OP(O)(OH)₂, and
      C₁-C₄alkyl, wherein said C₁-C₄ alkyl is optionally substituted with OH, OCH₃,
      C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 to 2 substituents independently selected from CH₃, halo, oxo and C(O)O(CH₃)₃,
   iii) C₃-C₅cycloalkyl,
   iv) halo,
   v) C(O)OCH₃,
   vi) P(O)(CH₃)₂, or
   vii) phenyl optionally substituted with 1 to 3 substituents independently selected from: halo, P(O)(CH₃)₂, and C₁-C₄alkyl, wherein the C₁-C₄alkyl is optionally substituted with OH; and
R¹⁴ and R¹⁵ are independently H or CH₃;
or a pharmaceutically acceptable salt thereof.

In an embodiment, there is provided a compound of Formula VI wherein
R¹ is:
   i) a 9- or 10-membered N-containing bicyclic heterocycle optionally substituted with 1 to 3 substituents independently selected from:
      C₃-C₄cycloalkyl optionally substituted with 1 or 2 halo or CH₃,
      4-membered heterocycle optionally substituted with CH₃,
      C₁-C₄alkoxy,
      C₁-C₄haloalkyl,
      oxo,
      CN
      halo, and
      C₁-C₄alkyl optionally substituted with OCH₃, OH or C₃-C₄cycloalkyl, wherein the C₃-C₄cycloalkyl is optionally substituted with 1 or 2 halo,
   ii) NHCOR¹², or
   iii) a 6-membered heterocycle optionally substituted with 1 or 2 substituents independently selected from: C(O)CH₃, C₁-C₄alkyl and oxo;
R¹² is NHR¹³ or C₃-C₆cycloalkyl optionally substituted with 1 or 2 halo;
R¹³ is phenyl optionally substituted with 1 or 2 halo;
A is phenyl, pyridyl, pyrimidinyl or thienyl, wherein the phenyl, pyridyl, pyrimidinyl or thienyl is optionally substituted with a substituent selected from: F, Cl, CF₃, CHF₂ and CH₃, or when R¹ is an optionally substituted 8-, 9- or 10-membered N-containing bicyclic heterocycle A may be absent;
R² is H or CH₃;
R³ is H and R⁴ is H, CH₃ or CD₃; or R³ and R⁴ together form —CH₂—CH₂—;
R⁵ is:
   i) a 9-membered N-containing bicyclic heterocycle, or
   ii) a 5-membered N-containing heteroaryl substituted with CH₃, CD₃ or CHF₂;
X is O or S;
R⁶ and R⁷ are each CH₃ or together form a cyclopropyl;
R⁹ is:

phenyl substituted with F, or a 5- or 6-membered N-containing heteroaryl optionally substituted with R¹⁰ or with R¹⁰ and R¹¹;
R¹⁰ is CF₃, CF₂H or F;
R¹¹ is:
   i) a 5- or 6-membered heterocycle optionally substituted with 1 to 4 substituents independently selected from:
      halo,
      OH,
      CD₃,
      OCHF₂,
      CN,
      C₁-C₄haloalkyl,
      C₃-C₅cycloalkyl,
      C(O)NH₂,
      a 4- to 6-membered heterocycle optionally substituted with oxo,
      oxo,
      CR¹⁴R¹⁵OP(O)(OH)₂, and
      C₁-C₄alkyl, wherein said C₁-C₄ alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 to 2 substituents independently selected from CH₃, halo, oxo and C(O)O(CH₃)₃,
   iii) C₃-C₅cycloalkyl,
   iv) halo,
   v) C(O)OCH₃, or
   vi) phenyl optionally substituted with 1 to 3 substituents independently selected from: halo and C₁-C₄alkyl, wherein the C₁-C₄alkyl is optionally substituted with OH; and
R¹⁴ and R¹⁵ are independently H or CH₃;
or a pharmaceutically acceptable salt thereof.

In an embodiment of Formula VI, there is a provided a compound of Formula VIa:

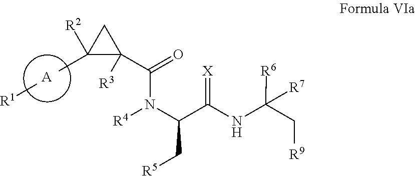

In an embodiment of Formula VI, R³ is H and R⁴ is H, CH₃ or CD₃. In an alternate embodiment of Formula VI, R³ and R⁴ together form —CH₂—CH₂—.

In an embodiment, there is a provided a compound of Formula VII:

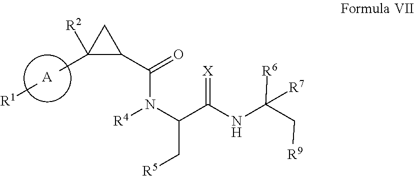

wherein R¹, A, R², R⁴, R⁵, X, R⁶, R⁷ and R⁹ are as defined in any of the above formulae.

In an embodiment of Formula VII, there is a provided a compound of Formula VIIa:

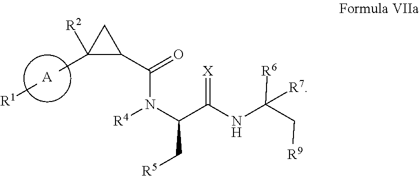

In a further embodiment of Formula VII, there is a provided a compound of Formula VIIb:

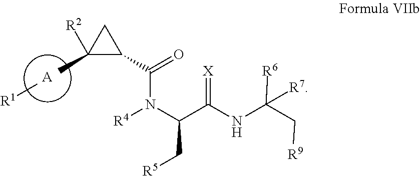

In an embodiment, there is a provided a compound of Formula VIII:

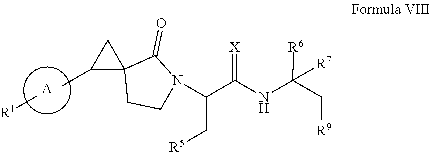

wherein R¹, A, R⁵, X, R⁶, R⁷ and R⁹ are as defined in any of the above formulae.

In an embodiment of Formula VIII, there is a provided a compound of Formula VIIIa:

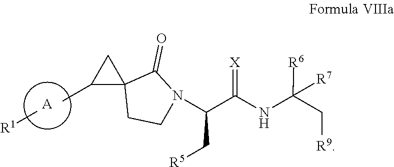

In an alternate embodiment, there is provided a compound of Formula I:

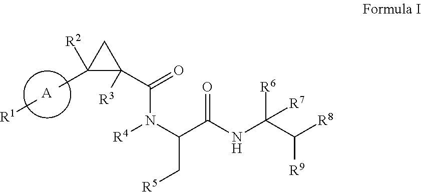

wherein
R¹ is:
   i) a 9- or 10-membered N-containing bicyclic heterocycle optionally substituted with 1 or 2 substituents independently selected from:
      C₃-C₄cycloalkyl optionally substituted with 1 or 2 halo or CH₃, C₁-C₄haloalkyl,
      oxo, and
      C₁-C₄alkyl optionally substituted with OCH₃ or C₃-C₄cycloalkyl, wherein the C₃-C₄cycloalkyl is optionally substituted with 1 or 2 halo,
   ii) NHCOR¹², or
   iii) OH;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
A is phenyl, a 6-membered N-containing heteroaryl or thienyl, wherein the phenyl, heteroaryl or thienyl is optionally substituted with 1 or 2 substituents independently selected from: halo, CF₃, CHF₂, CH₃ and cyclopropyl;
R² is H, CH₃, halo, CF₃ or CHF₂;
R³ is H and R⁴ is H or CH₃; or R³ and R⁴ together form —CH₂—CH₂—;
R⁵ is:
   i) a 8- or 9-membered N-containing bicyclic heterocycle optionally substituted with halo, or
   ii) a 5- or 6-membered N-containing heteroaryl optionally substituted with CH₃, CHF₂ or OH;
R⁶ and R⁷ are each CH₃ or together form a cyclopropyl;
R⁸ is H, CH₃, CO₂H or C(O)OCH₃ and R⁹ is:
   phenyl optionally substituted with 1 or 2 substituents selected from: halo, CF₃ and pyrazine optionally substituted with CF₂H or OCH₃, or
   a 5- or 6-membered N-containing heteroaryl optionally substituted with R¹⁰ or with R¹⁰ and R¹¹;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
   i) a 5- or 6-membered heterocycle optionally substituted with 1 to 3 substituents independently selected from:
      halo,
      OH,
      OCH₃,
      SO₂NH₂,
      a 4- to 6-membered heterocycle,
      oxo, and
      C₁-C₄alkyl, wherein the C₁-C₄alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with CN, oxo, C(O)O(CH₃)₃ or OH,
   iii) C₁-C₄alkoxy,
   iv) C₃-C₅cycloalkyl,
   v) halo, or
   vi) C(O)OCH₃;
or a pharmaceutically acceptable salt thereof.

In an embodiment of Formula I, there is a provided a compound of Formula Ia:

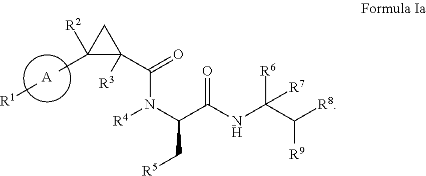

In an embodiment of Formula I, R⁶ and R⁷ are each CH₃.

In an embodiment of Formula I, R³ is H and R⁴ is H or CH₃. In an alternate embodiment of Formula I, R³ and R⁴ together form —CH₂—CH₂—.

In an embodiment of Formula I, R² is H or CH₃.

In an embodiment of Formula I, R¹ is:
i) a 9- or 10-membered N-containing bicyclic heteroaryl optionally substituted with 1 or 2 substituents independently selected from:
   C₃-C₄cycloalkyl optionally substituted with 1 or 2 halo or CH₃,
   C₁-C₄haloalkyl,
   C₁-C₄alkyl optionally substituted with OCH₃, or C₃-C₄cycloalkyl, wherein the C₃-C₄cycloalkyl is optionally substituted with 1 or 2 halo, or
ii) NHCOR¹², wherein R¹² is NHR¹³ or C₃-C₆cycloalkyl optionally substituted with 1 or 2 halo, and R¹³ is phenyl optionally substituted with 1 or 2 halo.

In an embodiment of Formula I, A is phenyl, pyridyl or thienyl, wherein the phenyl, pyridyl or thienyl is optionally substituted with a substituent selected from: F, CF₃ and CH₃.

In an embodiment of Formula I, R⁵ is:
i) a 9-membered N-containing bicyclic heterocycle, or
ii) a 5-membered N-containing heteroaryl substituted with CH₃ or CHF₂.

In an embodiment of Formula I, R⁸ is H.

In an embodiment of Formula I, R⁹ is:
phenyl substituted with F, or
a 5- or 6-membered N-containing heteroaryl optionally substituted with R¹⁰ or with R¹⁰ and R¹¹.

In an embodiment of Formula I, R⁹ is:
a 5- or 6-membered N-containing heteroaryl optionally substituted with R¹⁰ or with R¹⁰ and R¹¹;
wherein R¹⁰ is CF₃, CF₂H or F, and R¹¹ is:
   i) a 5- or 6-membered heterocycle optionally substituted with 1 or 2 substituents independently selected from:
      halo,
      a 4- to 6-membered heterocycle,
      oxo or
      C₁-C₄alkyl, wherein said C₁-C₄ alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with oxo or C(O)O(CH₃)₃,
   iii) C₃-C₅cycloalkyl,
   iv) halo, or
   v) C(O)OCH₃.

In a further alternate embodiment, there is a provided a compound of Formula II:

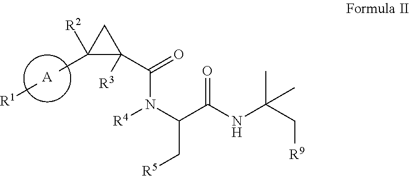

wherein
R¹ is:
   i) a 9- or 10-membered N-containing bicyclic heteroaryl optionally substituted with 1 or 2 substituents independently selected from:
      C₃-C₄cycloalkyl optionally substituted with 1 or 2 halo or CH₃,
      C₁-C₄haloalkyl, and
      C₁-C₄alkyl optionally substituted with OCH₃ or C₃-C₄cycloalkyl, wherein the C₃-C₄cycloalkyl is optionally substituted with 1 or 2 halo, or
   ii) NHCOR¹², wherein R¹² is NHR¹³ or C₃-C₆cycloalkyl optionally substituted with 1 or 2 halo and R¹³ is phenyl optionally substituted with 1 or 2 halo;
A is phenyl, pyridyl or thienyl, wherein the phenyl, pyridyl or thienyl is optionally substituted with a substituent selected from: F, CF₃ and CH₃;
R² is H or CH₃;
R³ is H and R⁴ is H or CH₃; or R³ and R⁴ together form —CH₂—CH₂—;
R⁵ is:
   i) a 9-membered N-containing bicyclic heterocycle, or
   ii) a 5-membered N-containing heteroaryl substituted with CH₃ or CHF₂;
R⁹ is:
phenyl substituted with F, or
a 5- or 6-membered N-containing heteroaryl optionally substituted with R¹⁰ or with R¹⁰ and R¹¹;
wherein R¹⁰ is CF₃, CF₂H or F, and R¹¹ is:
   i) a 5- or 6-membered heterocycle optionally substituted with 1 or 2 substituents independently selected from:
      halo,
      a 4- to 6-membered heterocycle,
      oxo, and
      C₁-C₄alkyl, wherein said C₁-C₄ alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with oxo or C(O)O(CH₃)₃,
   iii) C₃-C₅cycloalkyl,
   iv) halo, or
   v) C(O)OCH₃;
or a pharmaceutically acceptable salt thereof.

In an embodiment of Formula II, there is a provided a compound of Formula Ila:

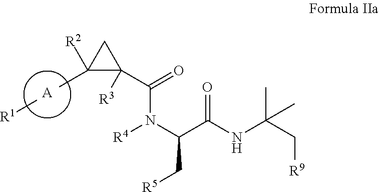

In an embodiment of Formula II, R³ is H and R⁴ is H or CH₃. In an alternate embodiment of Formula II, R³ and R⁴ together form —CH₂—CH₂—.

In a further alternate embodiment, there is a provided a compound of Formula III:

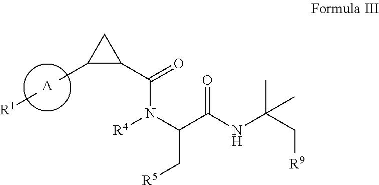

wherein R¹ is:
   i) a 9- or 10-membered N-containing bicyclic heteroaryl optionally substituted with 1 or 2 substituents independently selected from:
      C₃-C₄cycloalkyl optionally substituted with 1 or 2 halo or CH₃,
      C₁-C₄haloalkyl, and
      C₁-C₄alkyl optionally substituted with OCH₃ or C₃-C₄cycloalkyl, wherein the C₃-C₄cycloalkyl is optionally substituted with 1 or 2 halo, or
   ii) NHCOR¹², wherein R¹² is NHR¹³ or C₃-C₆cycloalkyl optionally substituted with 1 or 2 halo and R¹³ is phenyl optionally substituted with 1 or 2 halo;
A is phenyl, pyridyl or thienyl, wherein the phenyl, pyridyl or thienyl is optionally substituted with a substituent selected from: F, CF₃ and CH₃;
R⁴ is H or CH₃;
R⁵ is:
   i) a 9-membered N-containing bicyclic heterocycle, or
   ii) a 5-membered N-containing heteroaryl substituted with CH₃ or CHF₂;
R⁹ is:
phenyl substituted with F, or
a 5- or 6-membered N-containing heteroaryl optionally substituted with R¹⁰ or with R¹⁰ and R¹¹;
wherein R¹⁰ is CF₃, CF₂H or F, and R¹¹ is:
   i) a 5- or 6-membered heterocycle optionally substituted with 1 or 2 substituents independently selected from:
      halo,
      a 4- to 6-membered heterocycle,
      oxo, and
      C₁-C₄alkyl, wherein said C₁-C₄ alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with oxo or C(O)O(CH₃)₃,
   iii) C₃-C₅cycloalkyl,
   iv) halo, or
   v) C(O)OCH₃;
or a pharmaceutically acceptable salt thereof.

In an embodiment of Formula III, there is a provided a compound of Formula IIIa:

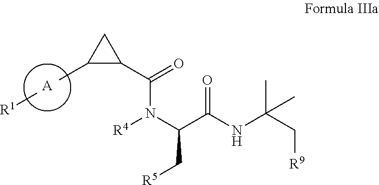

In a further embodiment of Formula III, there is a provided a compound of Formula IIIb:

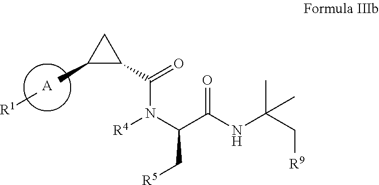

In a further alternate embodiment, there is a provided a compound of Formula IV:

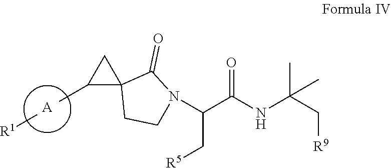

wherein R¹ is:
   i) a 9- or 10-membered N-containing bicyclic heteroaryl optionally substituted with 1 or 2 substituents independently selected from:
      C₃-C₄cycloalkyl optionally substituted with 1 or 2 halo or CH₃,
      C₁-C₄haloalkyl, and
      C₁-C₄alkyl optionally substituted with OCH₃ or C₃-C₄cycloalkyl, wherein the C₃-C₄cycloalkyl is optionally substituted with 1 or 2 halo, or
   ii) NHCOR¹², wherein R¹² is NHR¹³ or C₃-C₆cycloalkyl optionally substituted with 1 or 2 halo and R¹³ is phenyl optionally substituted with 1 or 2 halo;
A is phenyl, pyridyl or thienyl, wherein the phenyl, pyridyl or thienyl is optionally substituted with a substituent selected from: F, CF₃ and CH₃;
R⁵ is:
   i) a 9-membered N-containing bicyclic heterocycle, or
   ii) a 5-membered N-containing heteroaryl substituted with CH₃ or CHF₂;
R⁹ is:
phenyl substituted with F, or
a 5- or 6-membered N-containing heteroaryl optionally substituted with R¹⁰ or with R¹⁰ and R¹¹;
wherein R¹⁰ is CF₃, CF₂H or F, and R¹¹ is:
   i) a 5- or 6-membered heterocycle optionally substituted with 1 or 2 substituents independently selected from:
      halo,
      a 4- to 6-membered heterocycle,
      oxo, and
      C₁-C₄alkyl, wherein said C₁-C₄ alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with oxo or C(O)O(CH₃)₃,
   iii) C₃-C₅cycloalkyl,
   iv) halo, or
   v) C(O)OCH₃;
or a pharmaceutically acceptable salt thereof.

In an embodiment of Formula IV, there is a provided a compound of Formula IVa:

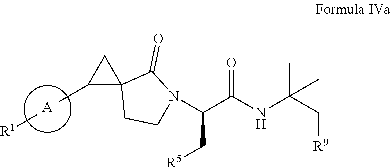

In a further embodiment of Formula IV, there is a provided a compound of Formula IVb:

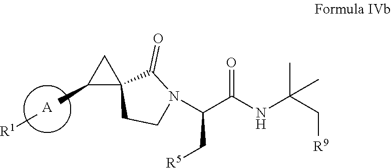

The embodiments below are embodiments of any or all of the applicable formulae above.

In an embodiment, X is O.

In an embodiment, R⁶ and R⁷ are each CH₃.

In an embodiment, R² is H or CH₃. Preferably, R² is H.

In an embodiment, R¹ is:
i) a 9- or 10-membered N-containing bicyclic heterocycle selected from:

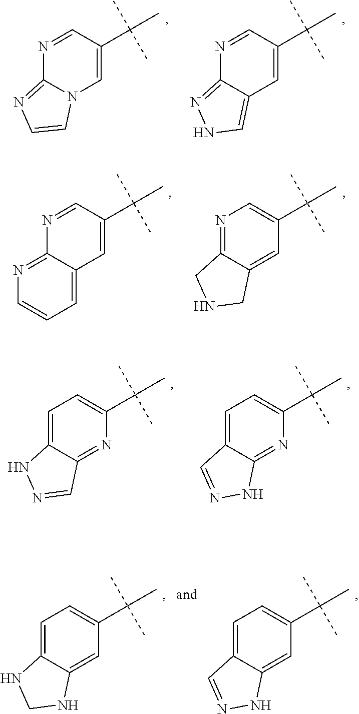

optionally substituted with 1 to 3 substituents independently selected from: C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo,
   CN,
   halo, and
   C₁-C₄alkyl optionally substituted with OCH₃, OH or C₃-C₄cycloalkyl, wherein the C₃-C₄cycloalkyl is optionally substituted with 1 or 2 halo,
   ii) NR¹⁶COR¹², wherein R¹² is NHR¹³, OCH₃ or C₃-C₆cycloalkyl optionally substituted with 1 or 2 halo, R¹³ is phenyl optionally substituted with 1 or 2 halo and R¹⁶ is H or CH₃, or
iii) piperazine optionally substituted with C(O)CH₃,
   pyridine optionally substituted with 1 or 2 substituents independently selected from OCH₃, halo and C₁-C₄alkyl optionally substituted with OH, or
   pyrimidine optionally substituted with 1 or 2 substituents independently selected from OCH₃, CH₃ and oxo.

In an embodiment, R¹ is:
i) a 9- or 10-membered N-containing bicyclic heterocycle selected from:

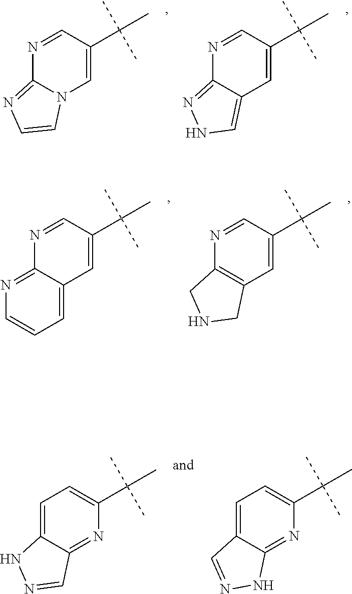

optionally substituted with 1 or 2 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo,
   CN,
   halo, and
   C₁-C₄alkyl optionally substituted with OCH₃, OH or C₃-C₄cycloalkyl, wherein the C₃-C₄cycloalkyl is optionally substituted with 1 or 2 halo,
ii) NHCOR¹², wherein R¹² is NHR¹³ or C₃-C₆cycloalkyl optionally substituted with 1 or 2 halo and R¹³ is phenyl optionally substituted with 1 or 2 halo, or
iii)

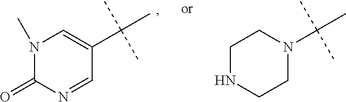

optionally substituted with C(O)CH₃.

In an embodiment, R¹ is:
i) a 9- or 10-membered N-containing bicyclic heterocycle selected from:

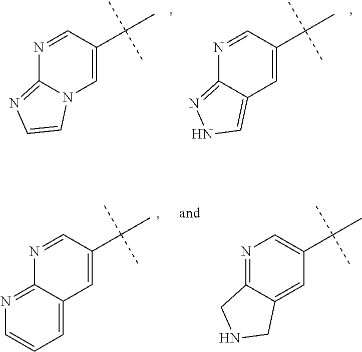

optionally substituted with 1 or 2 substituents independently selected from: C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo, and
   C₁-C₄alkyl optionally substituted with OCH₃, OH or C₃-C₄cycloalkyl, wherein the C₃-C₄cycloalkyl is optionally substituted with 1 or 2 halo,
ii) NHCOR¹², wherein R¹² is NHR¹³ or C₃-C₆cycloalkyl optionally substituted with 1 or 2 halo and R¹³ is phenyl optionally substituted with 1 or 2 halo, or
iii)

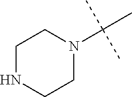

optionally substituted with C(O)CH₃.

In an alternate embodiment, R¹ is:
i) a 9- or 10-membered N-containing bicyclic heteroaryl selected from:

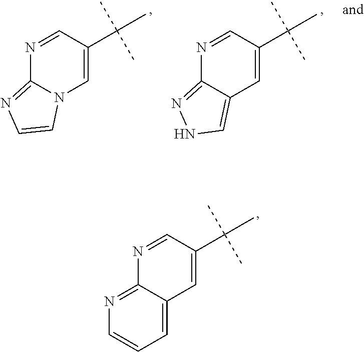

optionally substituted with 1 or 2 substituents independently selected from:
   C₃-C₄cycloalkyl optionally substituted with 1 or 2 halo or CH₃,
   C₁-C₄haloalkyl, and
   C₁-C₄alkyl optionally substituted with OCH₃ or C₃-C₄cycloalkyl, wherein the C₃-C₄cycloalkyl is optionally substituted with 1 or 2 halo, or
ii) NHCOR¹², wherein R¹² is NHR¹³ or C₃-C₆cycloalkyl optionally substituted with 1 or 2 halo and R¹³ is phenyl optionally substituted with 1 or 2 halo.

In an embodiment, R¹ is:
i) a 9- or 10-membered N-containing bicyclic heterocycle selected from:

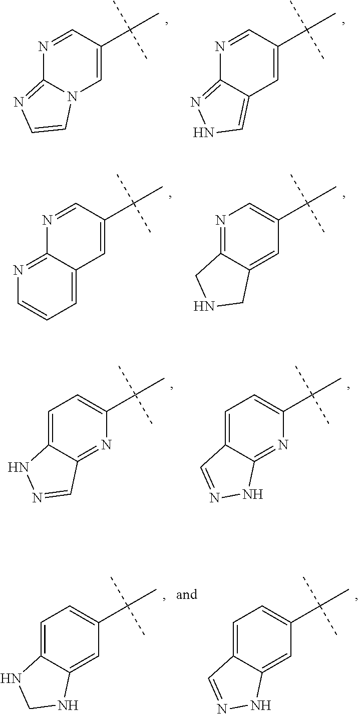

optionally substituted with 1 to 3 substituents independently selected from:
   cyclopropyl optionally substituted by CH₃,
   cyclobutyl optionally di-fluoro substituted,
   azetidinyl substituted with CH₃,
   OCH₃,
   CF₃,
   CHF₂,
   CF₂CH₃,
   oxo,
   CH₃,
   CH₂CH₃,
   CH₂OCH₃,
   (CH₂)₂OH,
   C(CH₃)₂OH,
   CH₂-cyclopropyl,
   CH₂-cyclobutyl,
   CN,
   fluoro, and
   chloro,
ii) NR¹⁶COR¹², wherein R¹² is

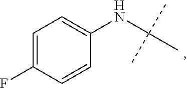

OCH₃, difluoro-substituted cyclohexyl or difluoro-substituted cyclobutyl, and R¹⁶ is H or CH₃, or
   iii) piperazine substituted with C(O)CH₃,
   pyridine substituted with 1 or 2 substituents independently selected from OCH₃, F and C(CH₃)₂OH, or
   pyrimidine substituted with 1 or 2 substituents independently selected from OCH₃, CH₃ and oxo.

In an embodiment, R¹ is:
i) a 9- or 10-membered N-containing bicyclic heterocycle selected from:

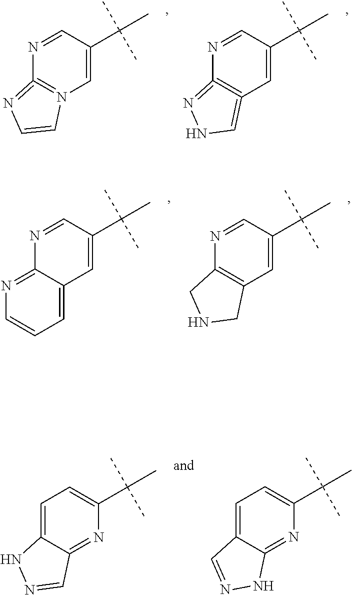

optionally substituted with 1 to 3 substituents independently selected from:
   cyclopropyl optionally substituted by CH₃,
   cyclobutyl optionally di-fluoro substituted,
   azetidinyl substituted with CH₃,
   OCH₃,
   CF₃,
   CHF₂,
   CF₂CH₃,
   oxo,
   CH₃,
   CH₂CH₃,
   CH₂OCH₃,
   (CH₂)₂OH,
   C(CH₃)₂OH,
   CH₂-cyclopropyl,
   CN,
   fluoro, and
   chloro,
ii) NHCOR¹², wherein R¹² is

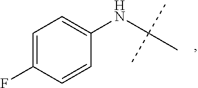

difluoro-substituted cyclobutyl or difluoro-substituted cyclobutyl, or

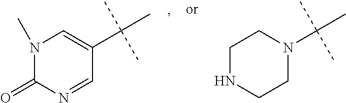

iii) substituted with C(O)CH₃.

In an embodiment, R¹ is:
i) a 9- or 10-membered N-containing bicyclic heterocycle selected from:

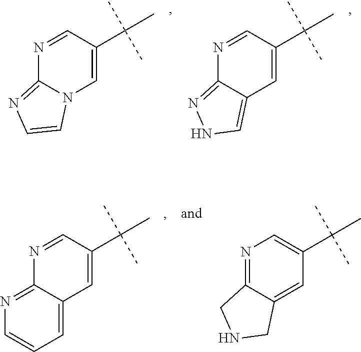

optionally substituted with 1 or 2 substituents independently selected from:
   cyclopropyl optionally substituted by CH₃,
   cyclobutyl optionally di-fluoro substituted,
   azetidinyl substituted with CH₃,
   OCH₃,
   CF₃,
   CHF₂,
   CF₂CH₃,
   oxo,
   CH₃,
   CH₂CH₃,
   CH₂OCH₃,
   (CH₂)₂OH,
   C(CH₃)₂OH, and
   CH₂-cyclopropyl,
ii) NHCOR¹², wherein R¹² is

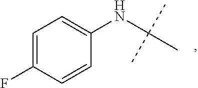

difluoro-substituted cyclobutyl or difluoro-substituted cyclobutyl, or
iii)

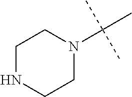

substituted with C(O)CH₃.

In an alternate embodiment, R¹ is:
i) a 9- or 10-membered N-containing bicyclic heteroaryl selected from:

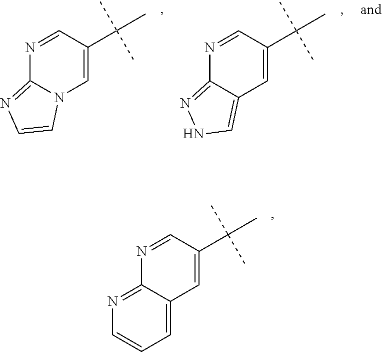

optionally substituted with 1 or 2 substituents independently selected from: cyclopropyl optionally substituted by CH₃, cyclobutyl optionally di-fluoro substituted, CF₃, CHF₂, CF₂CH₃, CH₃, CH₂CH₃, CH₂OCH₃ and CH₂-cyclopropyl, or
ii) NHCOR¹², wherein R¹² is

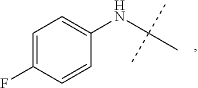

difluoro-substituted cyclobutyl or difluoro-substituted cyclobutyl.

In a further embodiment, R¹ is selected from:

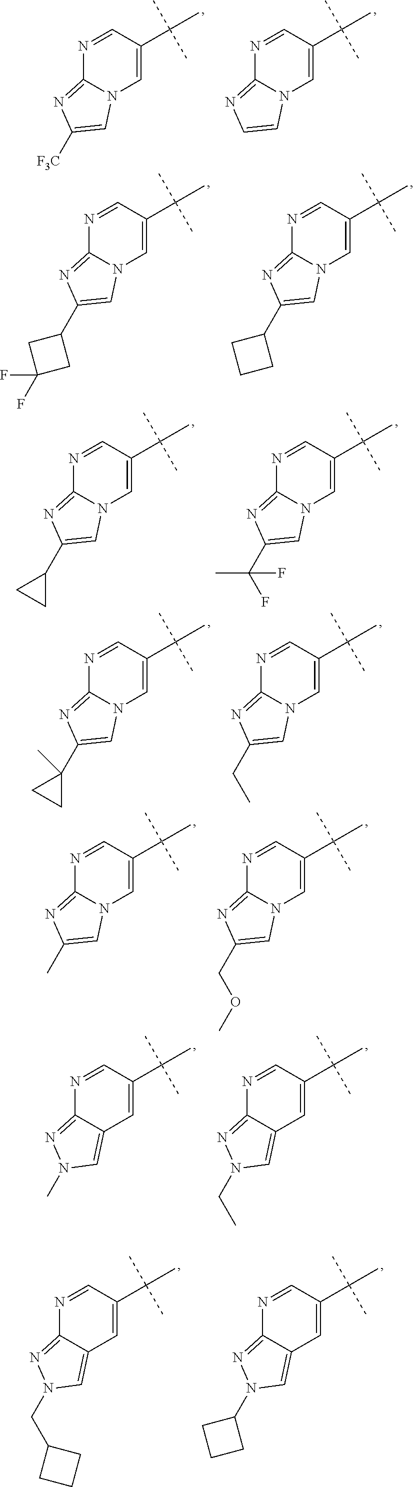

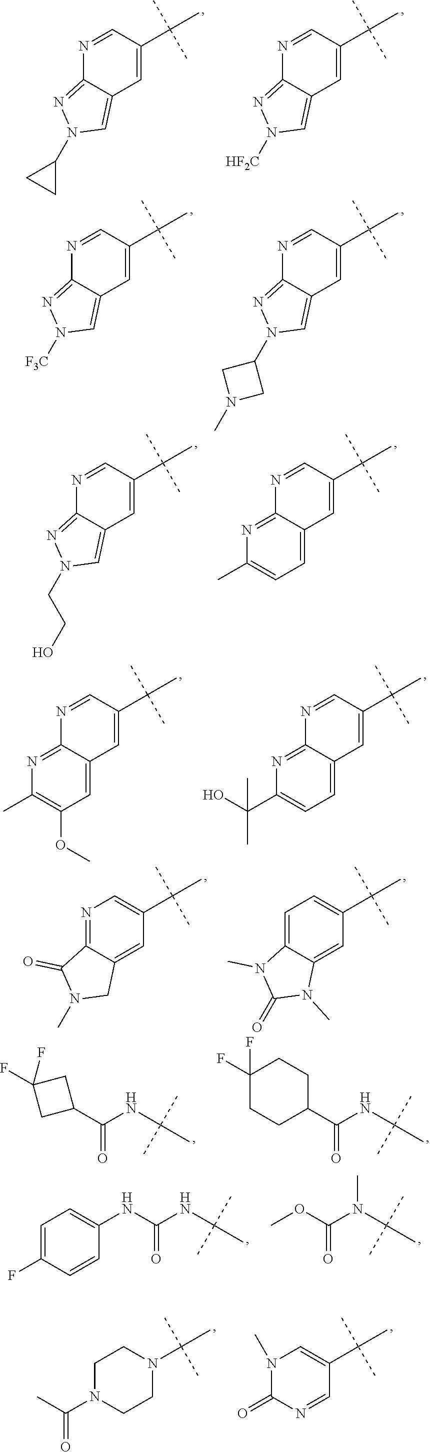

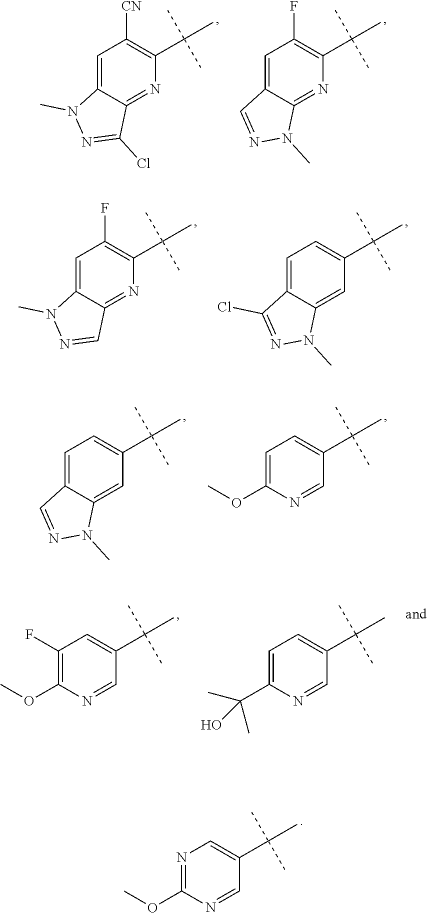

In a further embodiment, R¹ is selected from:

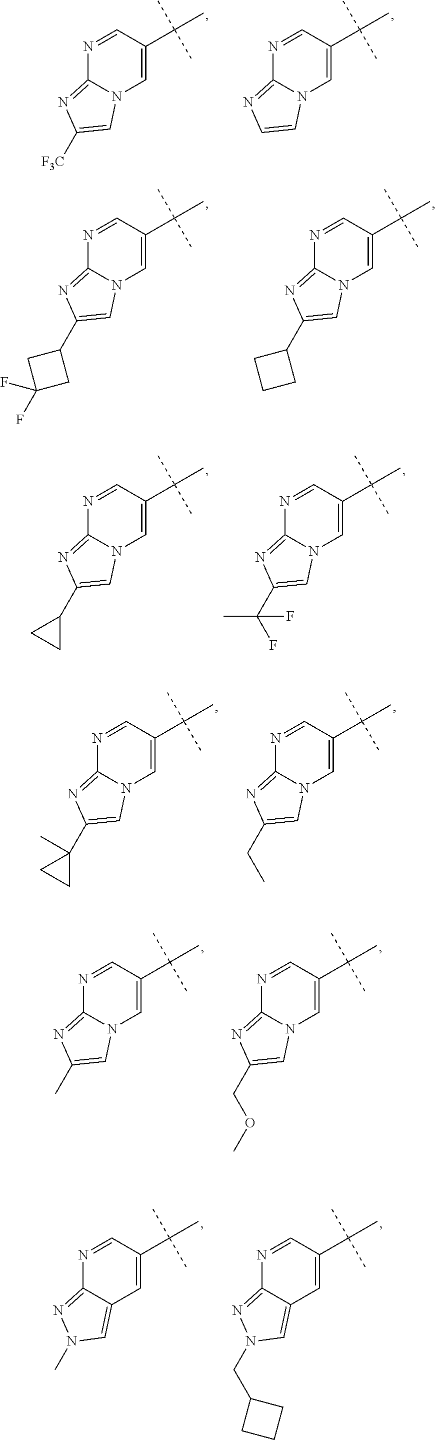

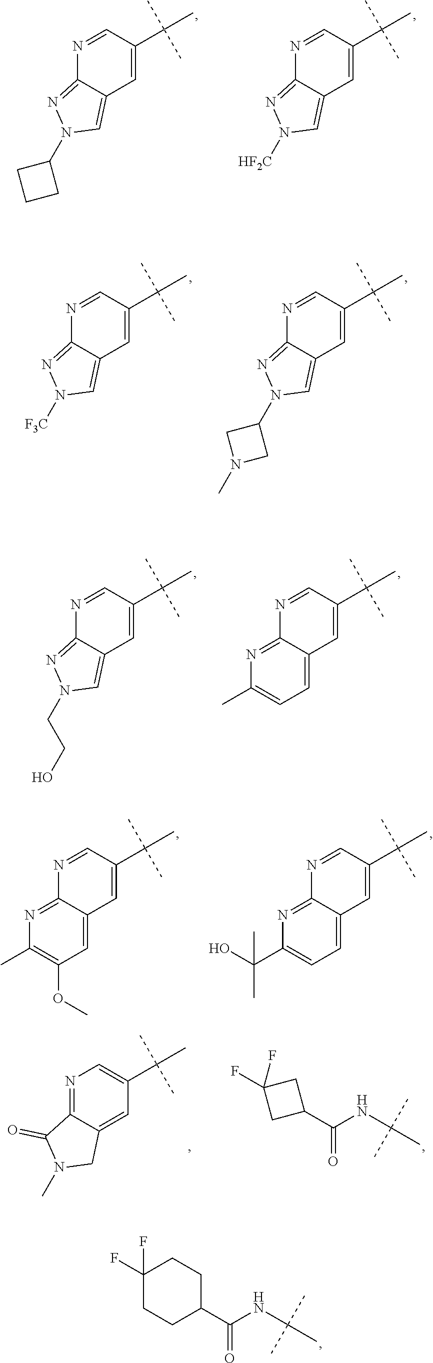

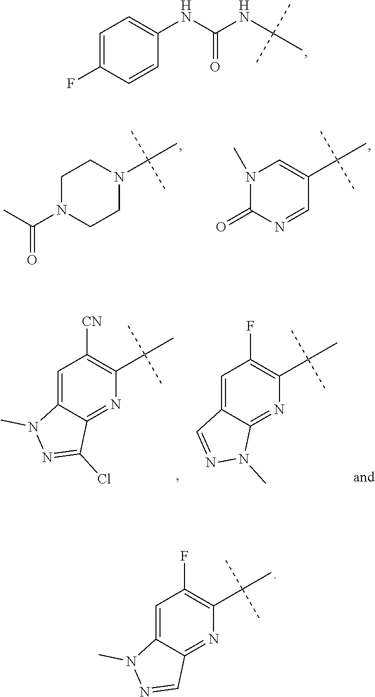

In a further embodiment, R¹ is selected from:

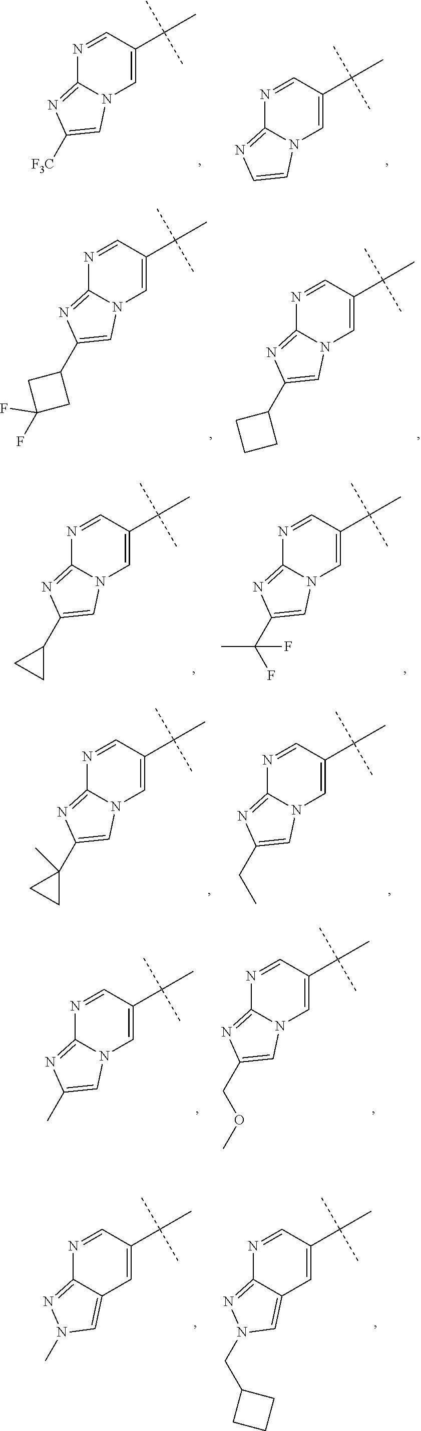

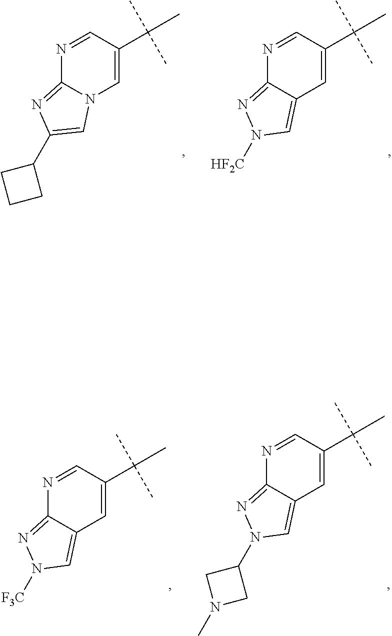

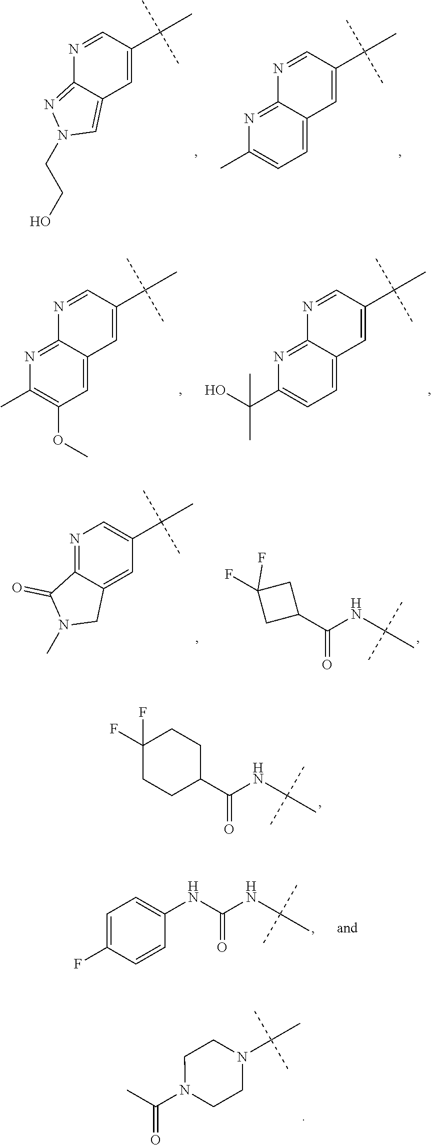

In an alternate embodiment, R¹ is selected from:

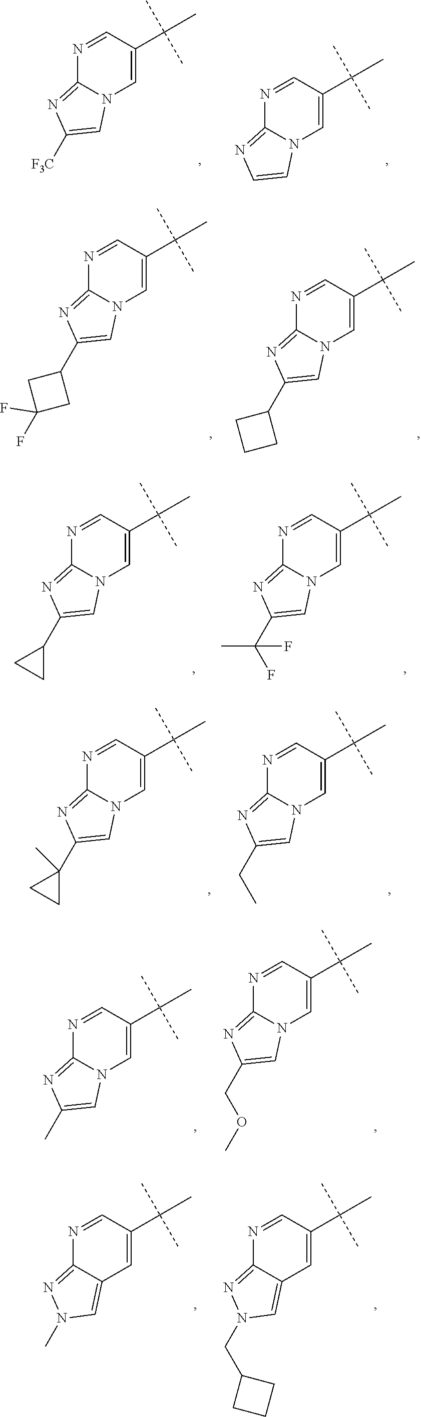

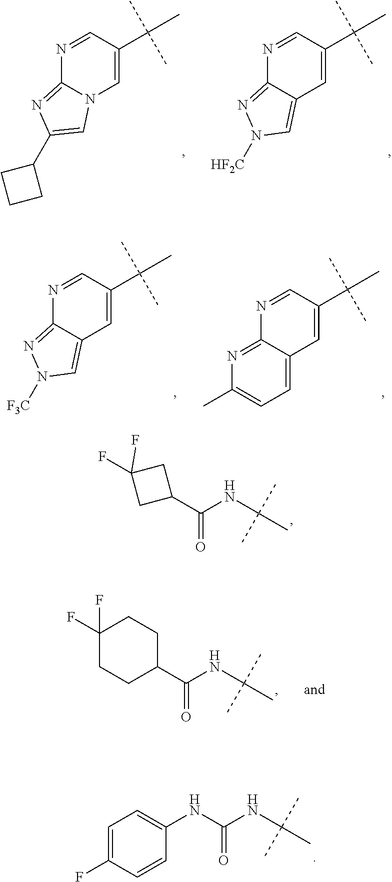

In a further embodiment, R¹ is selected from:

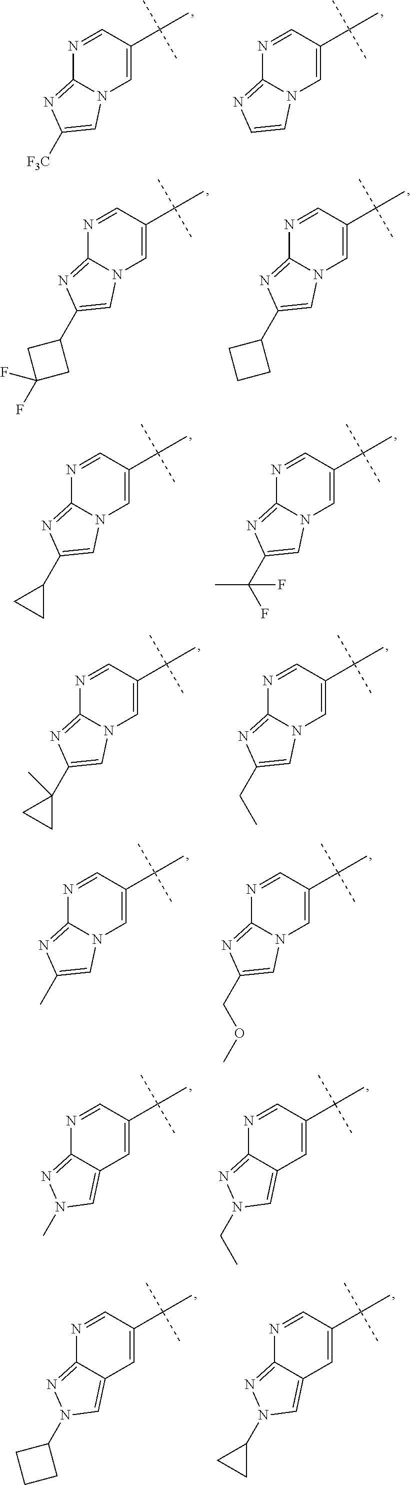

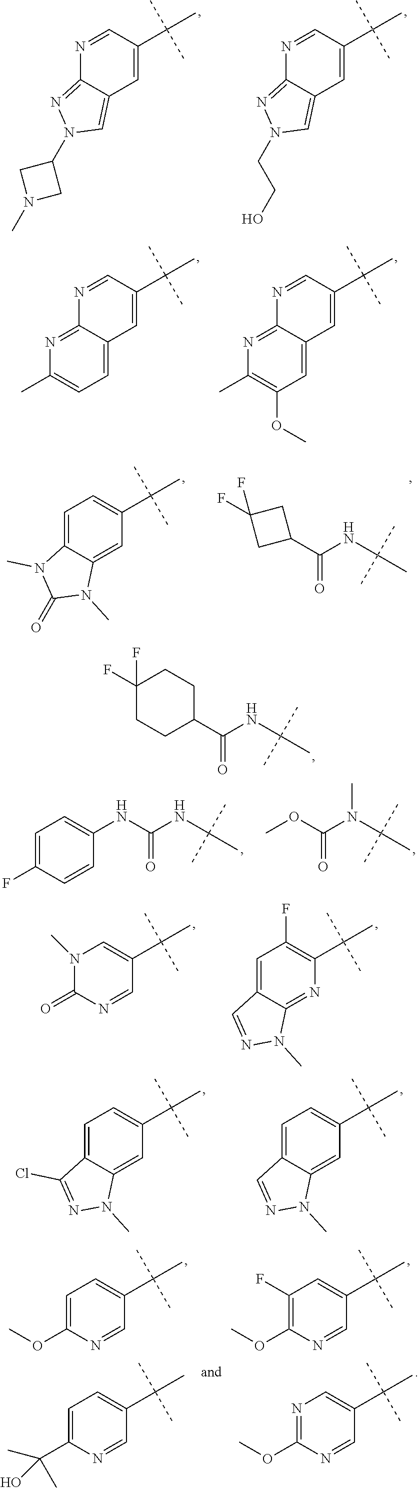

In a further embodiment, R¹ is selected from:

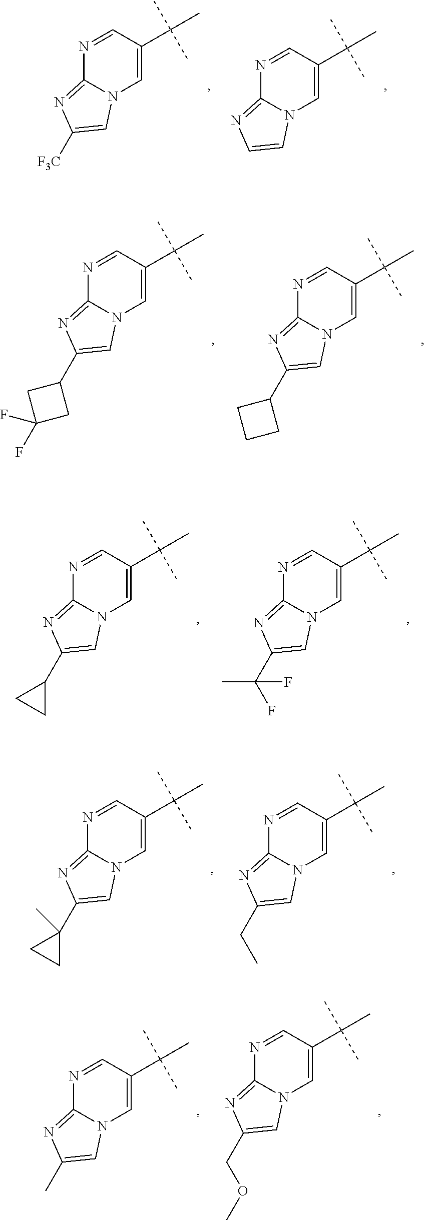

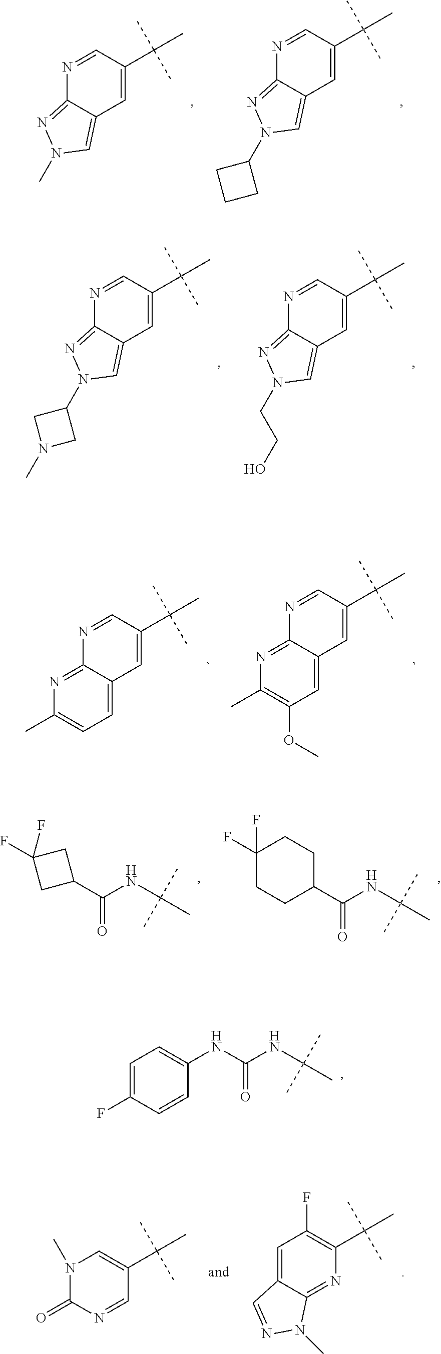

In a further embodiment, R¹ is selected from:

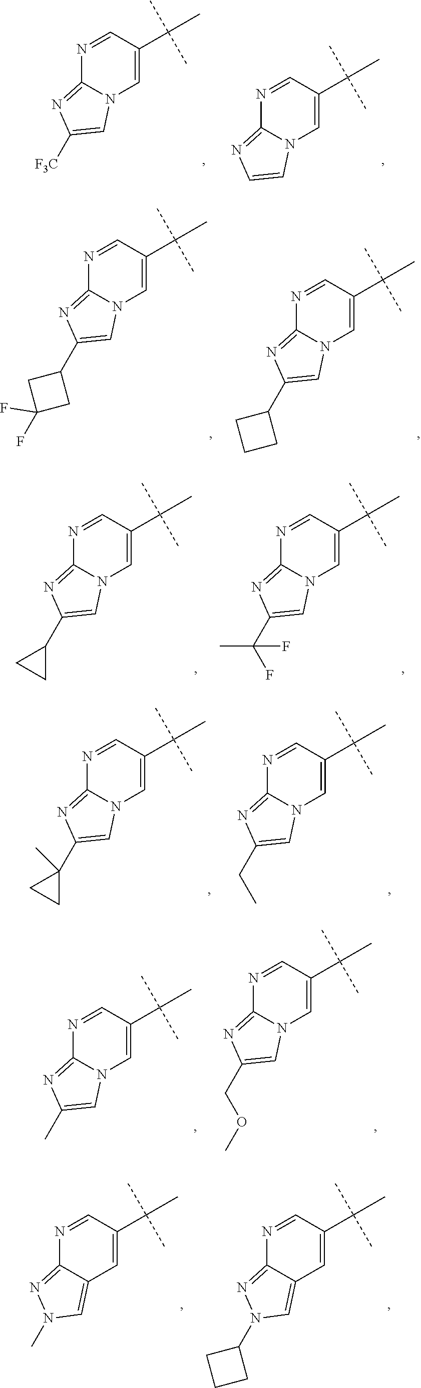

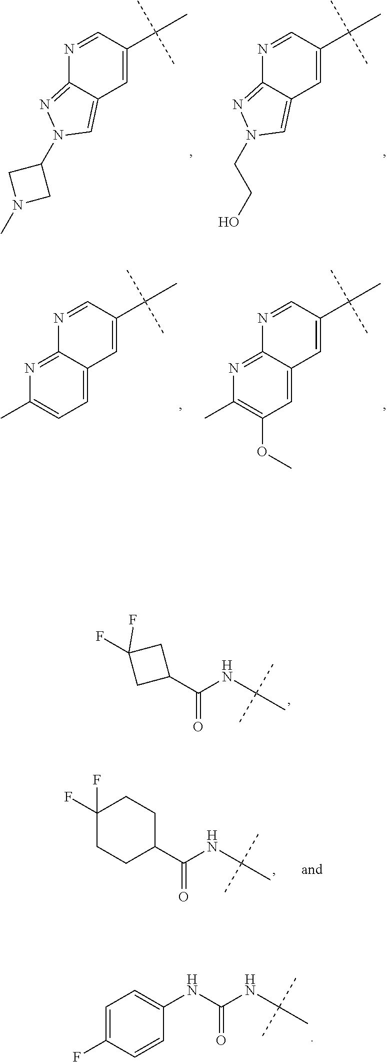

In an alternate embodiment, R¹ is selected from:

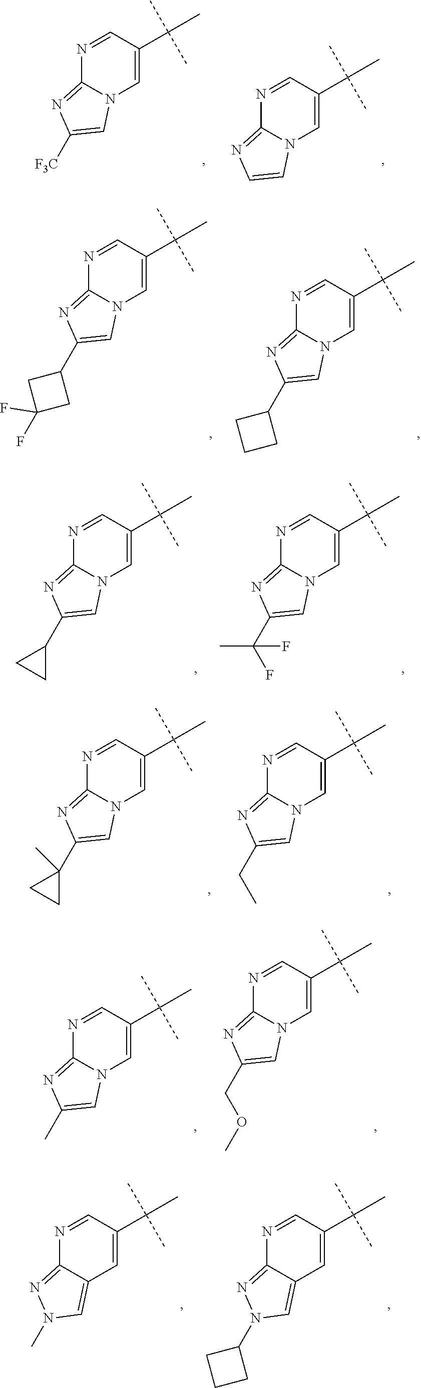

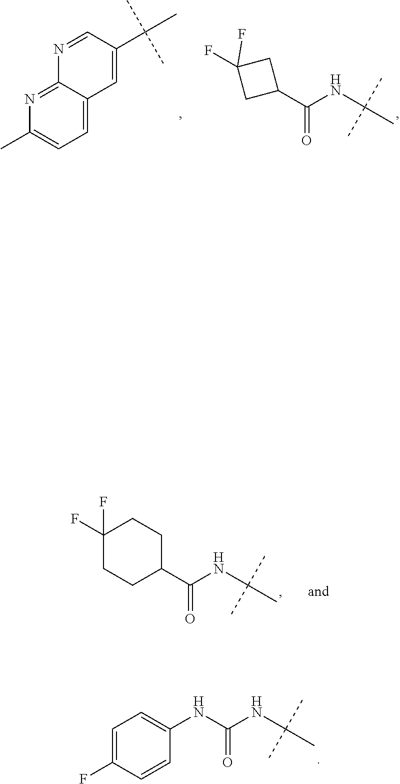

In an embodiment, R¹ is:
a 9- or 10-membered N-containing bicyclic heterocycle optionally substituted with 1 to 3 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo,
   CN,
   halo, and
   C₁-C₄alkyl optionally substituted with OCH₃, OH or C₃-C₄cycloalkyl, wherein the C₃-C₄cycloalkyl is optionally substituted with 1 or 2 halo.

In an embodiment, R¹ is:
a 9- or 10-membered N-containing bicyclic heterocycle optionally substituted with 1 or 2 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo, and
   C₁-C₄alkyl optionally substituted with OCH₃, OH or C₃-C₄cycloalkyl, wherein the C₃-C₄cycloalkyl is optionally substituted with 1 or 2 halo.

In an alternate embodiment, R¹ is:
a 9- or 10-membered N-containing bicyclic heteroaryl optionally substituted with 1 or 2 substituents independently selected from:
   C₃-C₄cycloalkyl optionally substituted with 1 or 2 halo or CH₃,
   C₁-C₄haloalkyl, and
   C₁-C₄alkyl optionally substituted with OCH₃ or C₃-C₄cycloalkyl, wherein the C₃-C₄cycloalkyl is optionally substituted with 1 or 2 halo.

In an embodiment, R¹ is:
a 9- or 10-membered N-containing bicyclic heterocycle selected from:

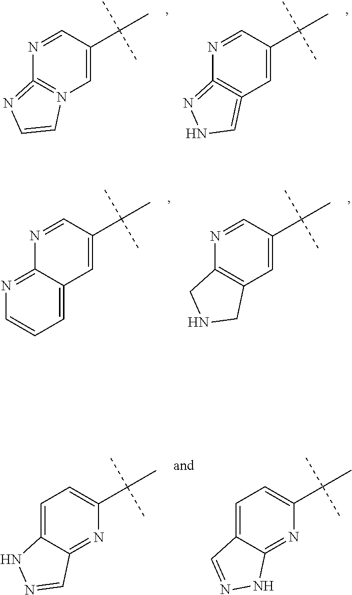

optionally substituted with 1 or 2 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo,
   CN,
   halo, and
   C₁-C₄alkyl optionally substituted with OCH₃, OH or C₃-C₄cycloalkyl, wherein the C₃-C₄cycloalkyl is optionally substituted with 1 or 2 halo.

In an embodiment, R¹ is:
a 9- or 10-membered N-containing bicyclic heterocycle selected from:

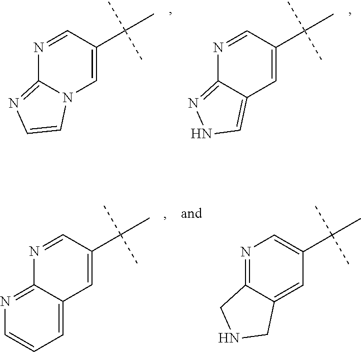

optionally substituted with 1 or 2 substituents independently selected from:
   C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo, and
   C₁-C₄alkyl optionally substituted with OCH₃, OH or C₃-C₄cycloalkyl, wherein the C₃-C₄cycloalkyl is optionally substituted with 1 or 2 halo.

In an alternate embodiment, R¹ is:
a 9- or 10-membered N-containing bicyclic heteroaryl selected from:

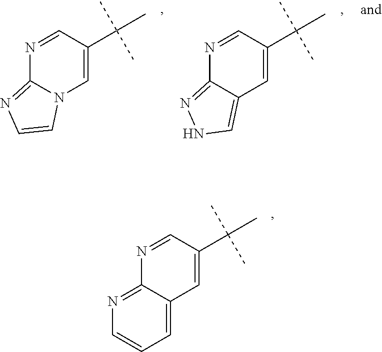

optionally substituted with 1 or 2 substituents independently selected from:
   C₃-C₄cycloalkyl optionally substituted with 1 or 2 halo or CH₃,
   C₁-C₄haloalkyl, and
   C₁-C₄alkyl optionally substituted with OCH₃ or C₃-C₄cycloalkyl, wherein the C₃-C₄cycloalkyl is optionally substituted with 1 or 2 halo.

In an embodiment, R¹ is:
a 9- or 10-membered N-containing bicyclic heterocycle selected from:

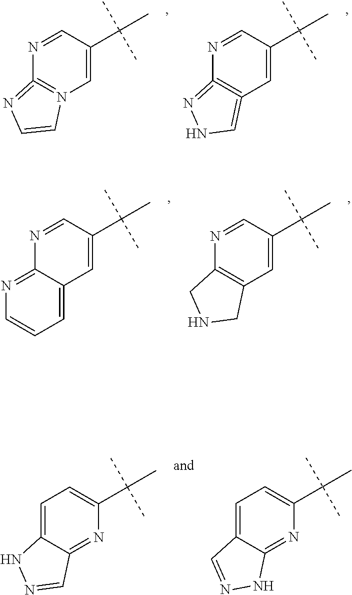

optionally substituted with 1 to 3 substituents independently selected from:
   cyclopropyl optionally substituted by CH₃,
   cyclobutyl optionally di-fluoro substituted,
   azetidinyl substituted with CH₃,
   OCH₃,
   CF₃,
   CHF₂,
   CF₂CH₃,
   oxo,
   CH₃,
   CH₂CH₃,
   CH₂OCH₃,
   (CH₂)₂OH,
   C(CH₃)₂OH,
   CH₂-cyclopropyl,
   CN,
   fluoro, and
   chloro.

In an embodiment, R¹ is:
a 9- or 10-membered N-containing bicyclic heterocycle selected from:

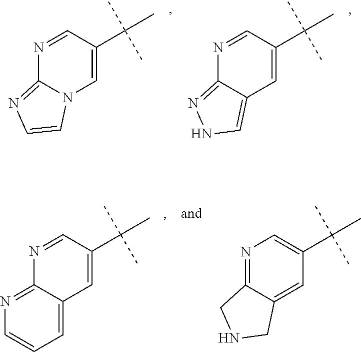

optionally substituted with 1 or 2 substituents independently selected from:
   cyclopropyl optionally substituted by CH₃,
   cyclobutyl optionally di-fluoro substituted,
   azetidinyl substituted with CH₃,
   OCH₃
   CF₃,
   CHF₂,
   CF₂CH₃,
   oxo,
   CH₃,
   CH₂CH₃,
   CH₂OCH₃,
   (CH₂)₂OH,
   C(CH₃)₂OH, and
   CH₂-cyclopropyl.

In an alternate embodiment, R¹ is:
a 9- or 10-membered N-containing bicyclic heteroaryl selected from:

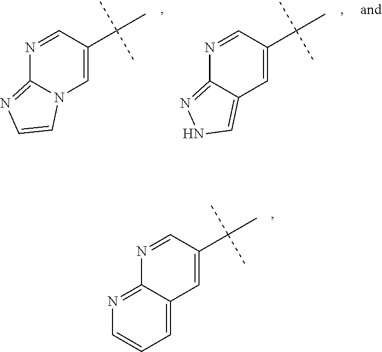

optionally substituted with 1 or 2 substituents independently selected from: cyclopropyl optionally substituted by CH₃, cyclobutyl optionally di-fluoro substituted, CF₃, CHF₂, CF₂CH₃, CH₃, CH₂CH₃, CH₂OCH₃ and CH₂-cyclopropyl.

In a further embodiment, R¹ is selected from:

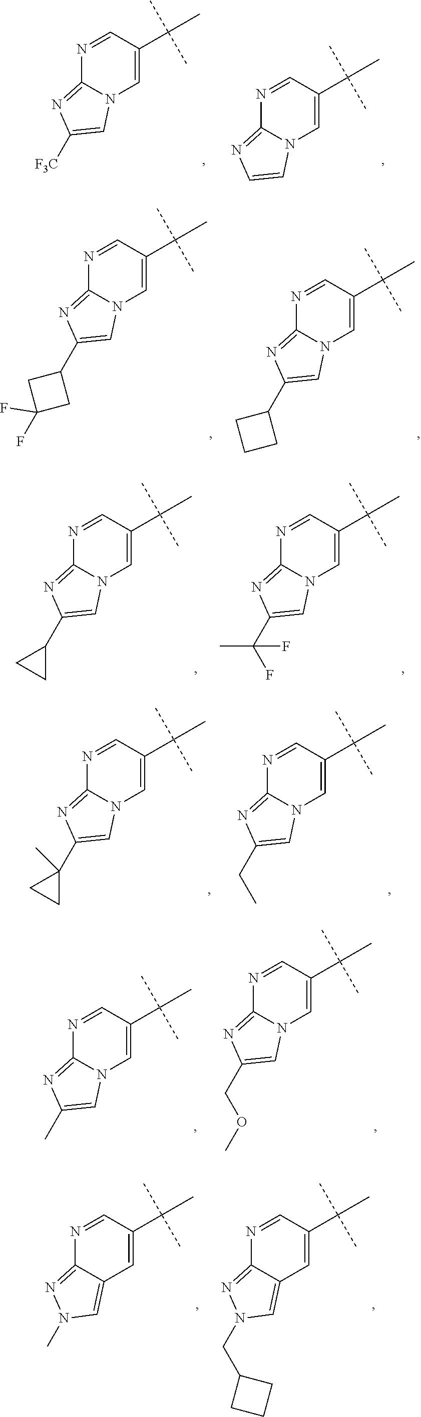

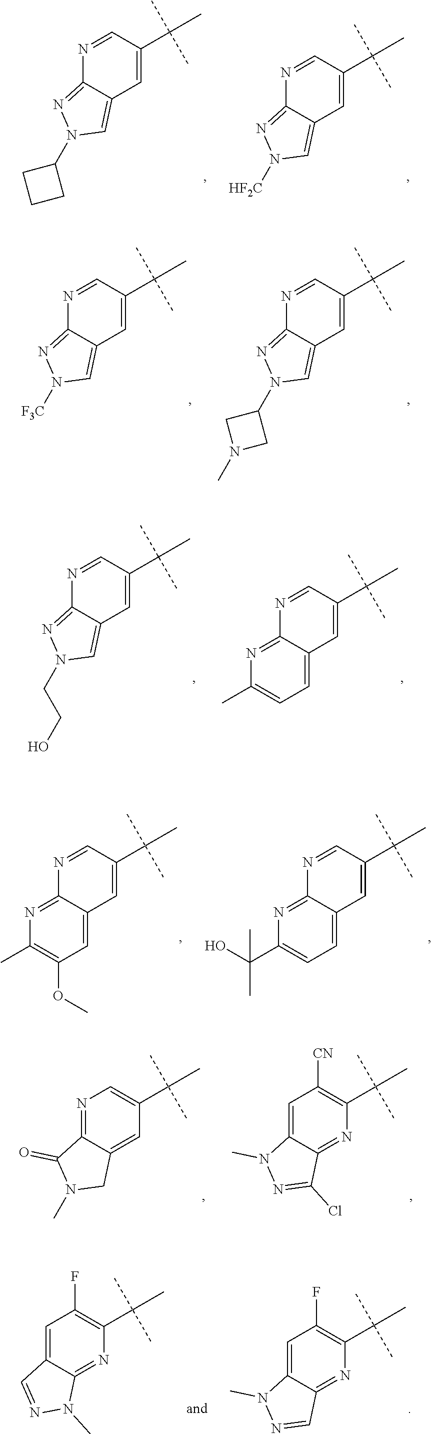

In a further embodiment, R¹ is selected from:

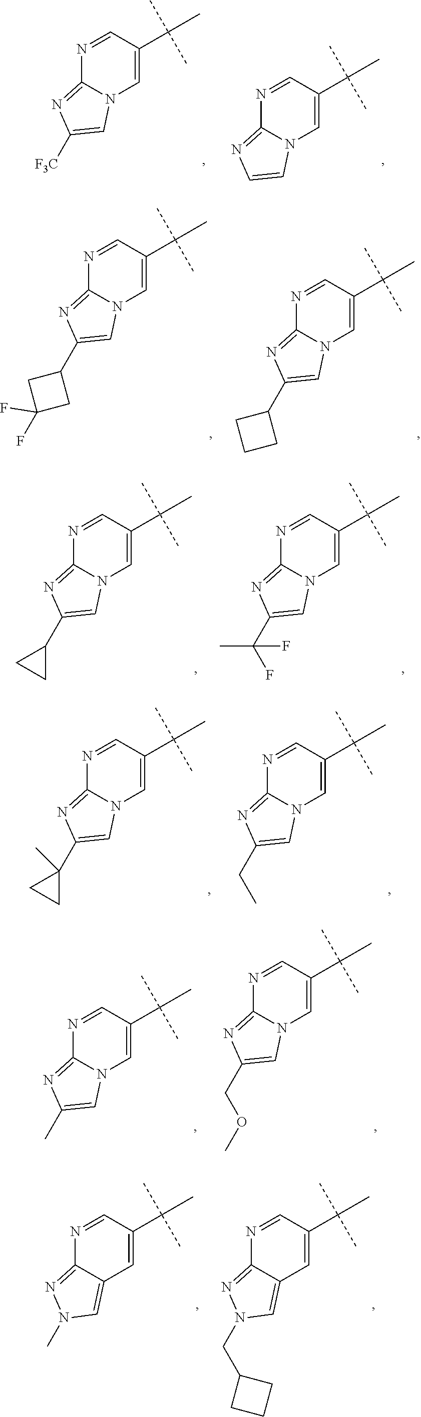

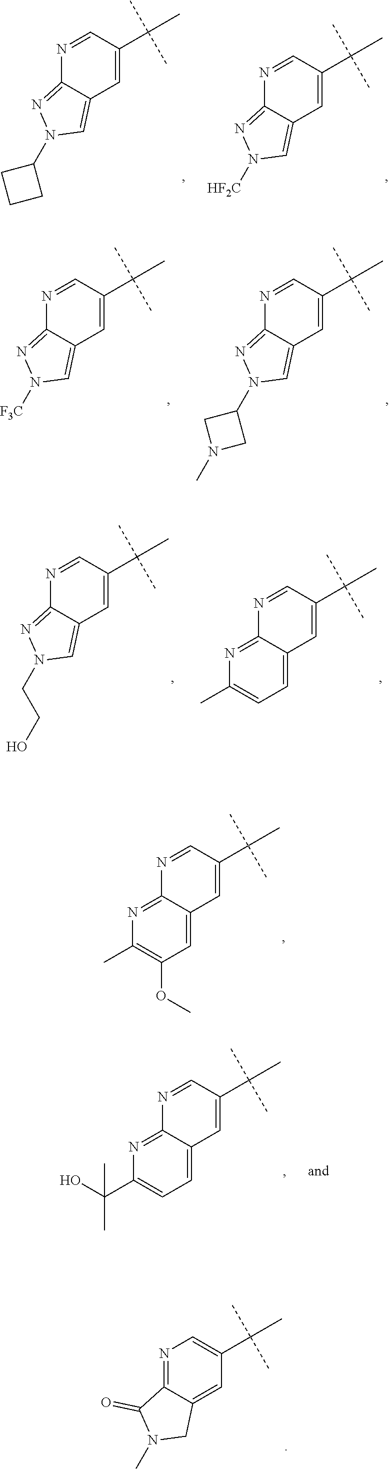

In an alternate embodiment, R¹ is selected from:

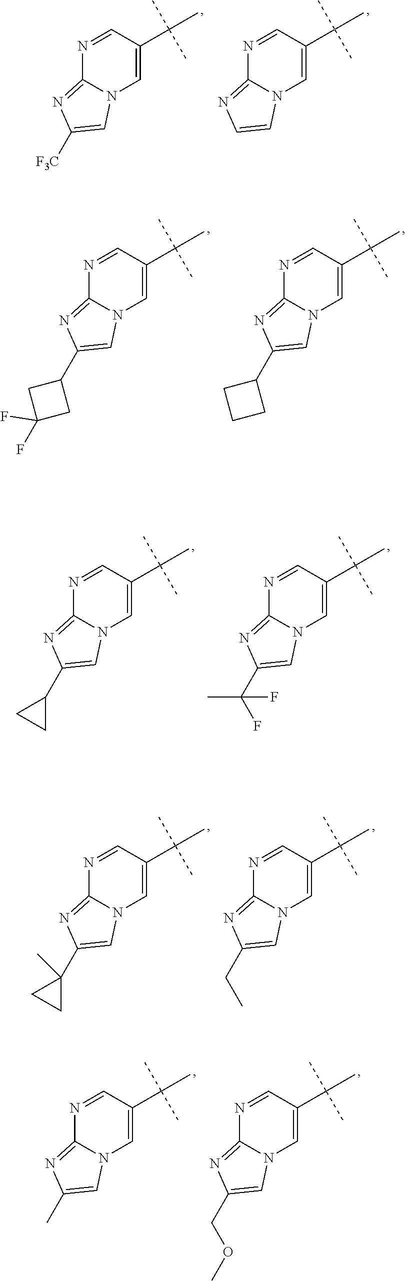

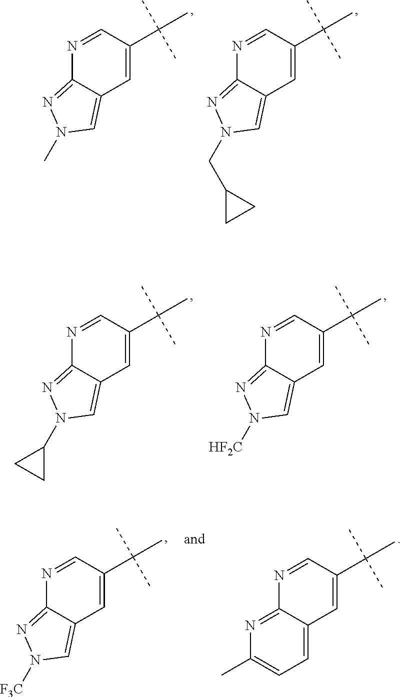

In a further embodiment, R¹ is selected from:

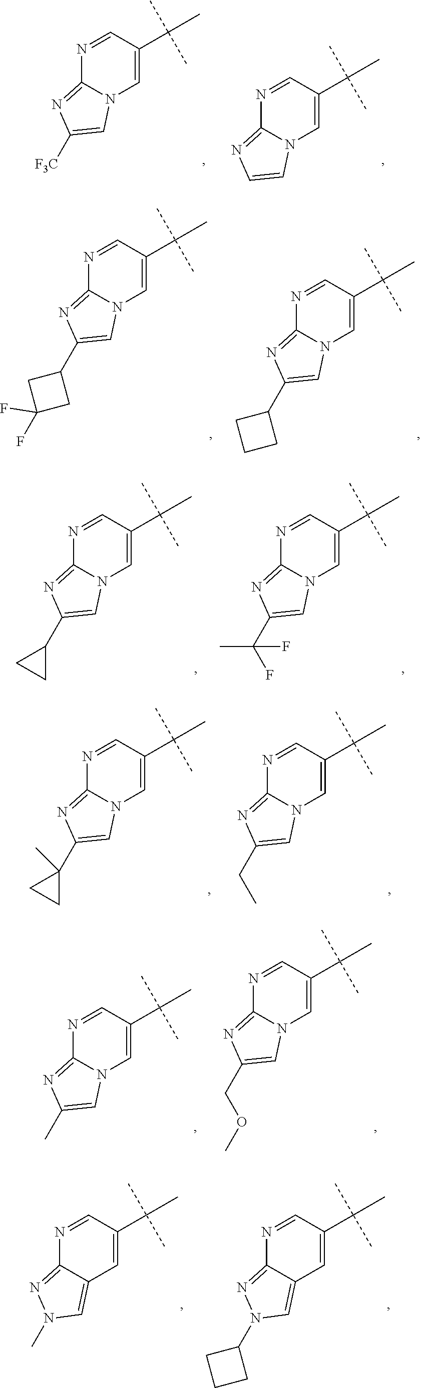

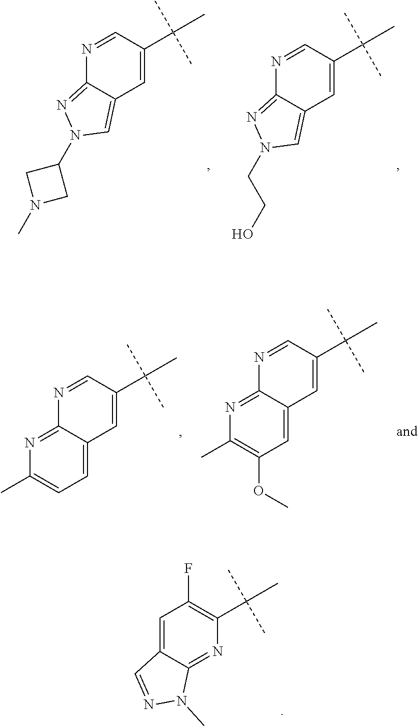

In a further embodiment, R¹ is selected from:

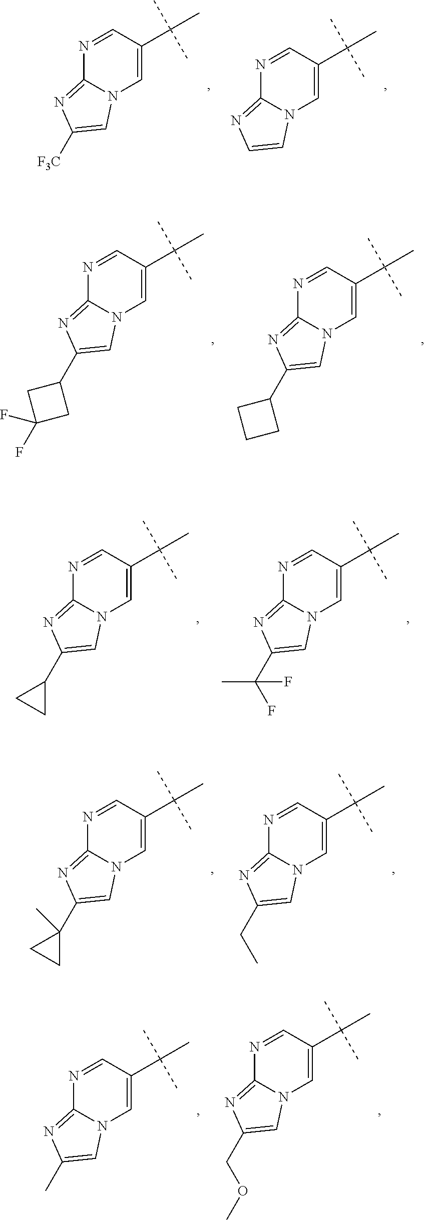

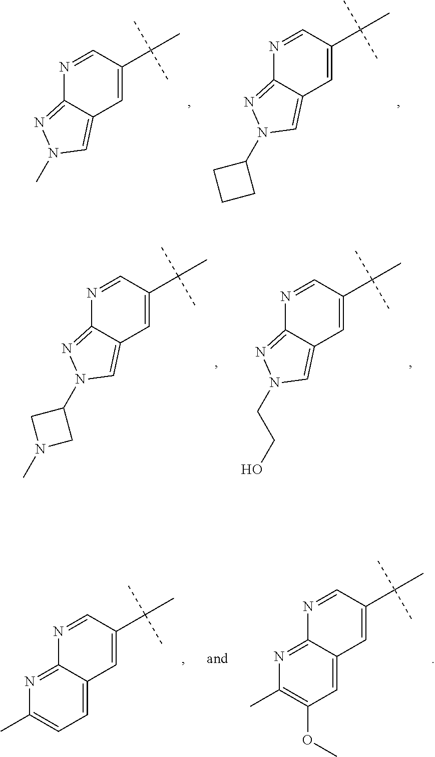

In an alternate embodiment, R¹ is selected from:

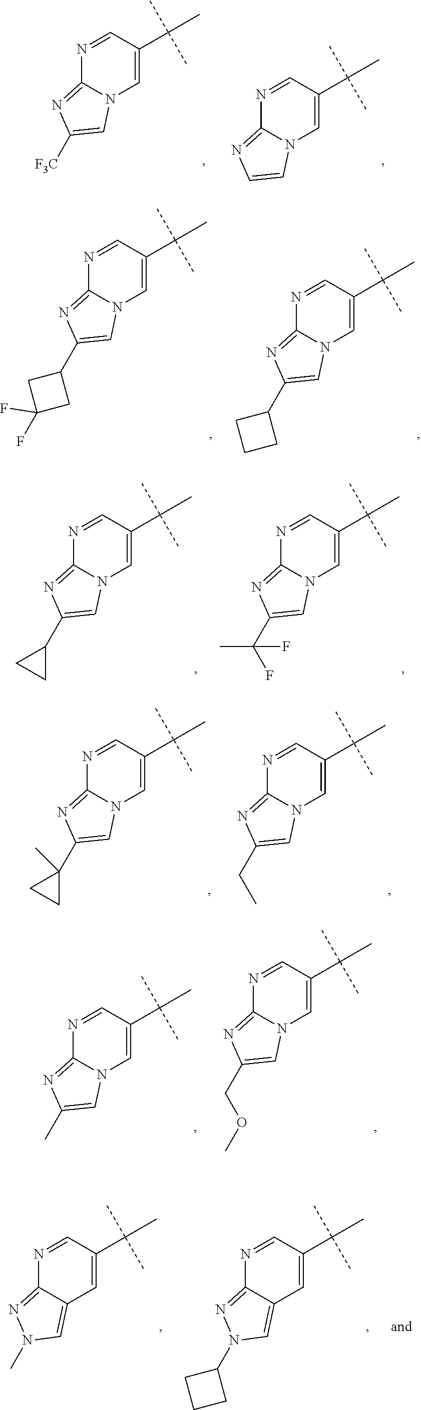

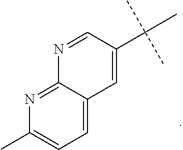

In a specific embodiment. R¹ is

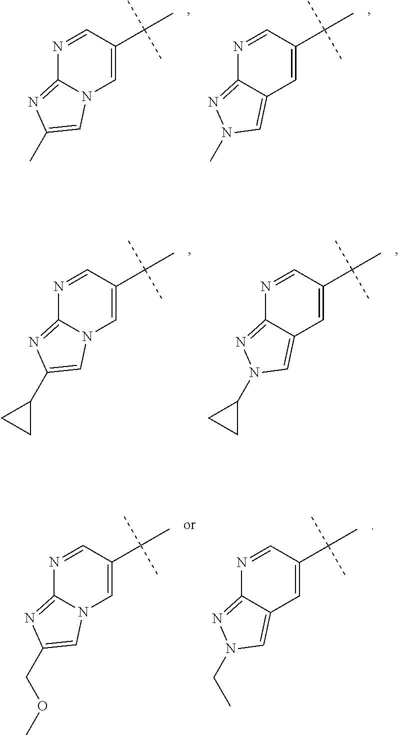

In a specific embodiment, R¹ is

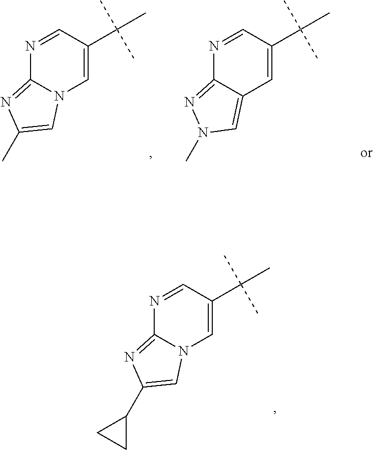

In a specific embodiment, R¹ is

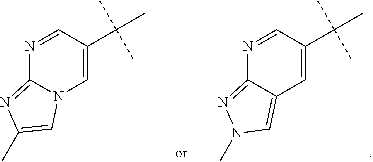

In a particular embodiment, R¹ is:
NHCOR¹², wherein R¹² is NHR¹³ or C₃-C₆cycloalkyl optionally substituted with 1 or 2 halo and R¹³ is phenyl optionally substituted with 1 or 2 halo.

In a further embodiment, R¹ is:
NHCOR¹², wherein R¹² is

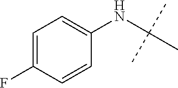

difluoro-substituted cyclobutyl or difluoro-substituted cyclobutyl.

In a further embodiment, R¹ is selected from:

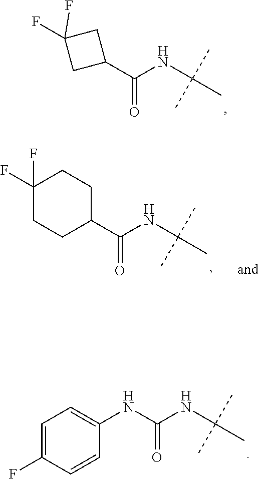

In a further embodiment, A is selected from:

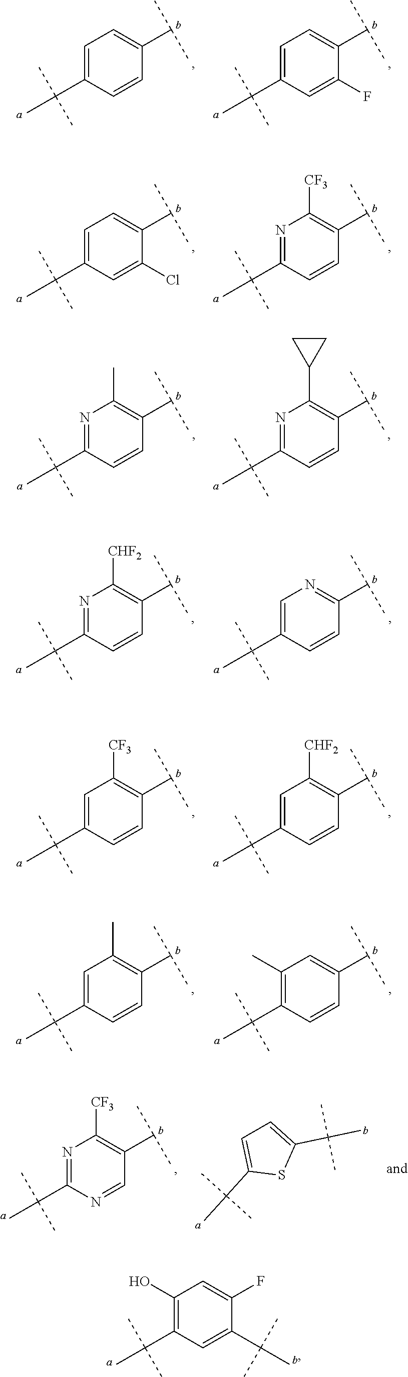

wherein a is the point of attachment to R¹ and b is the point of attachment to the cyclopropyl, or A is absent. In a further embodiment, A is selected from:

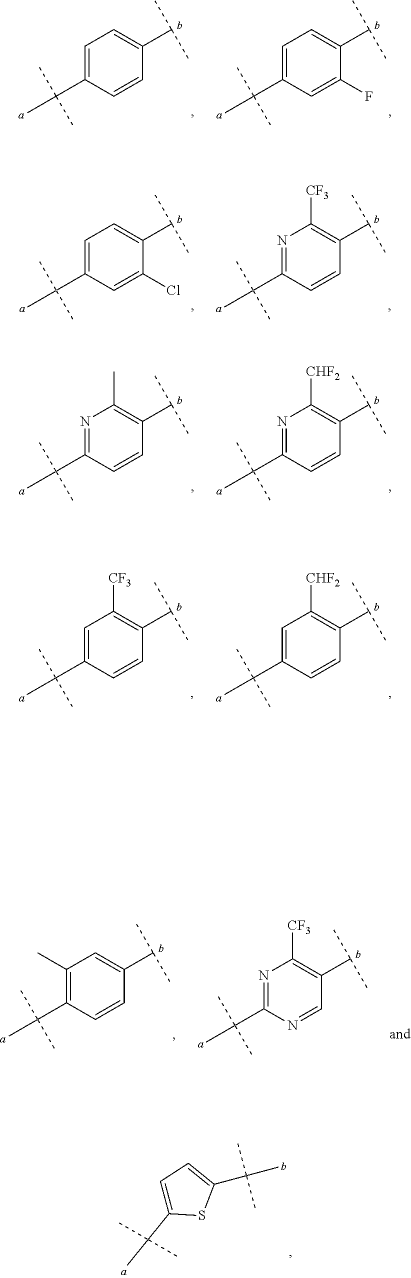

wherein a is the point of attachment to R¹ and b is the point of attachment to the cyclopropyl. In a further embodiment, A is selected from:

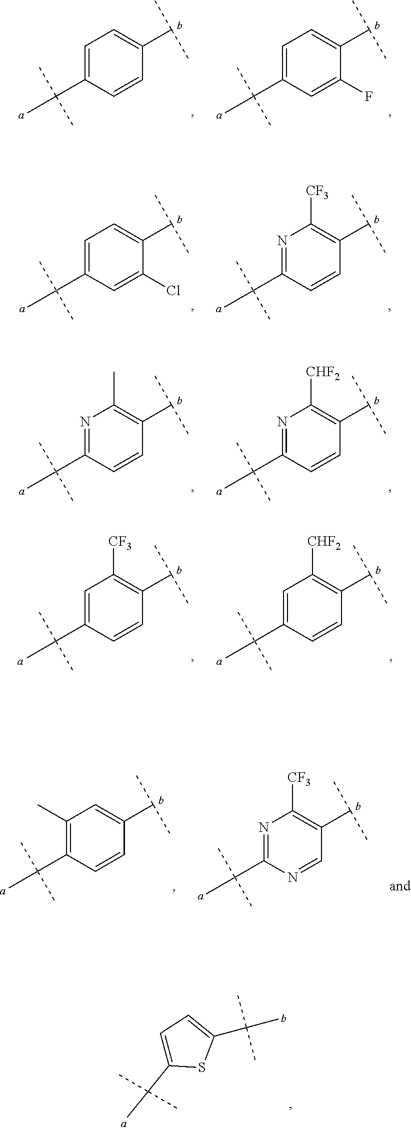

wherein a is the point of attachment to R¹ and b is the point of attachment to the cyclopropyl. In an alternate embodiment, A is selected from:

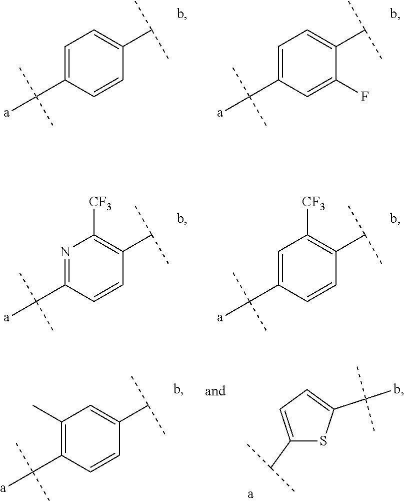

wherein a is the point of attachment to R¹ and b is the point of attachment to the cyclopropyl.

In a further embodiment, A is selected from:

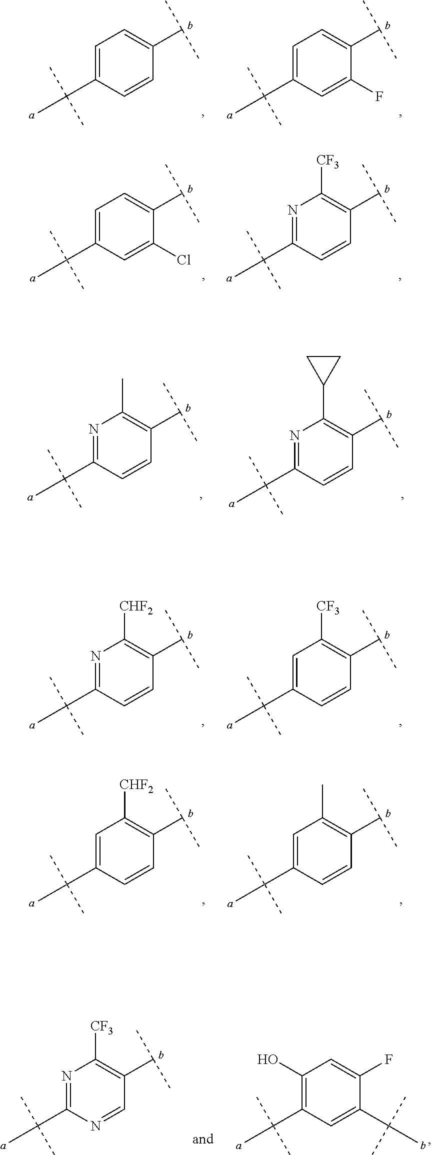

wherein a is the point of attachment to R¹ and b is the point of attachment to the cyclopropyl, or A is absent. In a further embodiment, A is selected from:

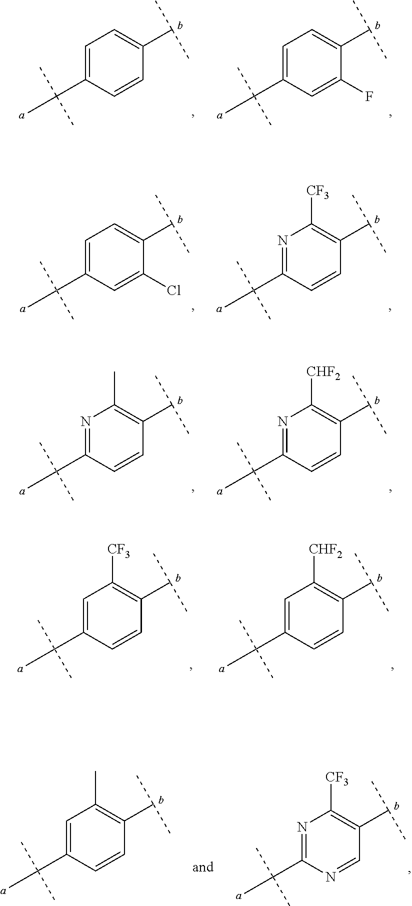

wherein a is the point of attachment to R¹ and b is the point of attachment to the cyclopropyl. In a further embodiment, A is selected from:

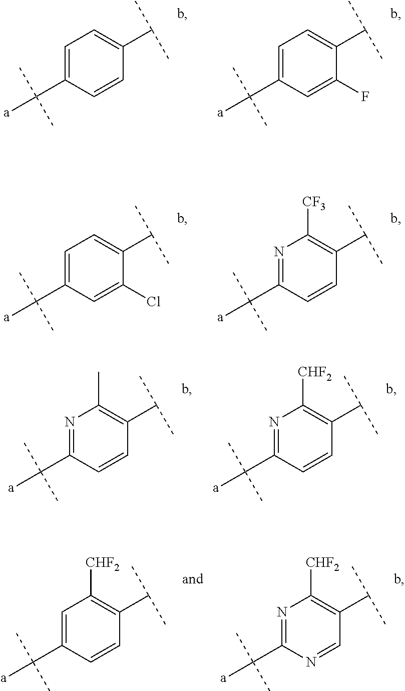

wherein a is the point of attachment to R¹ and b is the point of attachment to the cyclopropyl. In an alternate embodiment, A is selected from:

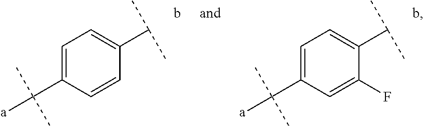

wherein a is the point of attachment to R¹ and b is the point of attachment to the cyclopropyl.

In a specific embodiment, A is

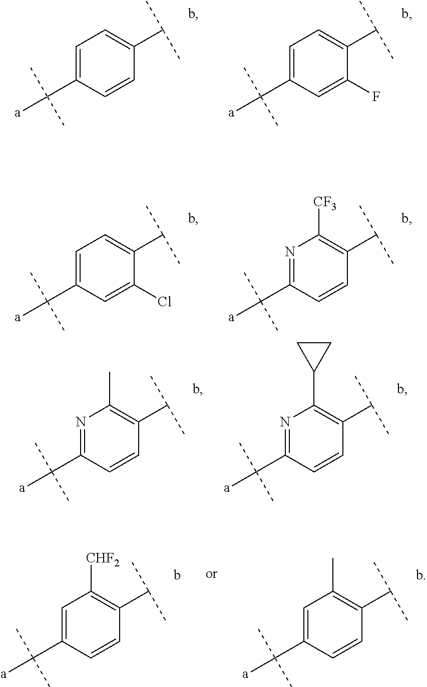

In a specific embodiment, A is

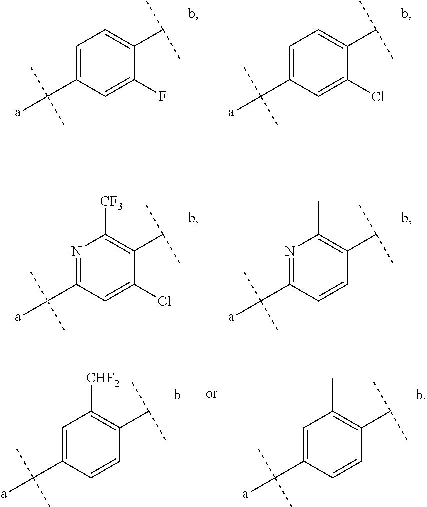

In a specific embodiment, A is

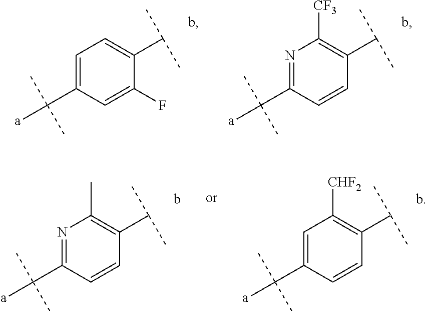

In a further embodiment, R⁵ is:

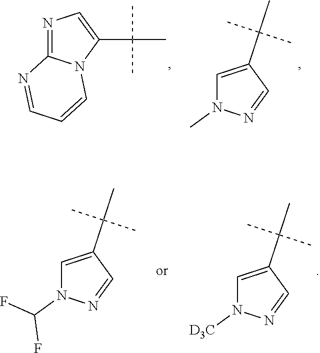

In an alternate embodiment, R⁵ is:

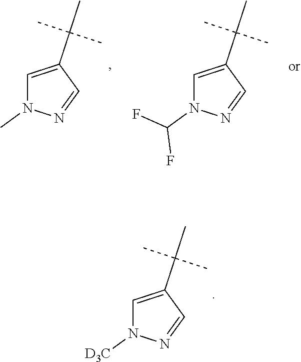

In an alternate embodiment, R⁵ is:

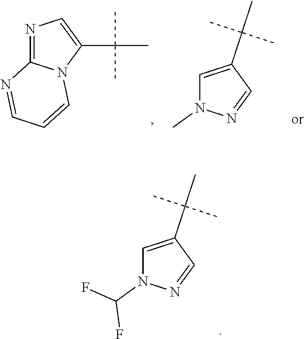

In a specific embodiment, R⁵ is

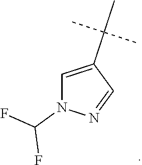

In an embodiment, R⁸ is H.

In a further embodiment, R⁹ is:

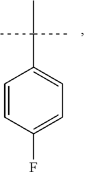

or
a 5- or 6-membered N-containing heteroaryl selected from:

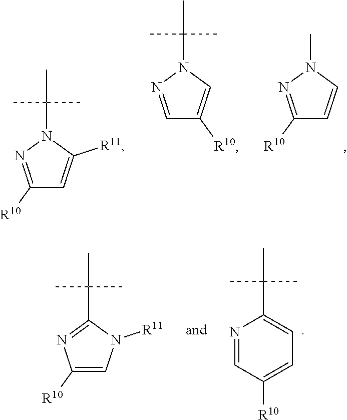

In a further embodiment, R⁹ is:

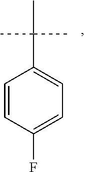

or
a 5- or 6-membered N-containing heteroaryl selected from:

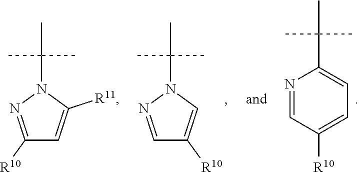

In a further embodiment, R⁹ is:

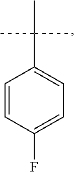

or
a 5- or 6-membered N-containing heteroaryl selected from:

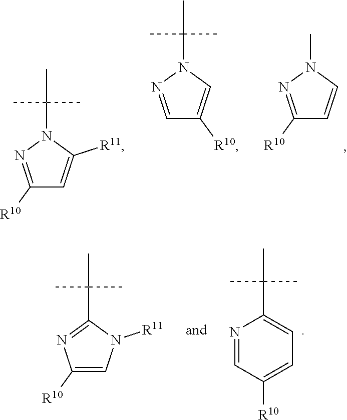

wherein R¹⁰ is CF₃, CF₂H or F, and R¹¹ is:
i) a 5- or 6-membered heterocycle selected from:

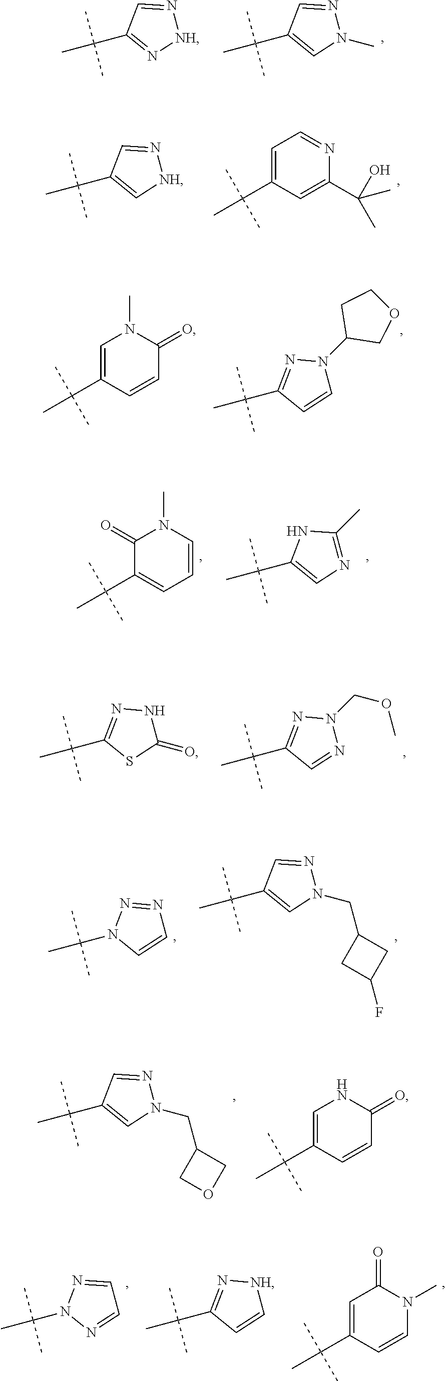

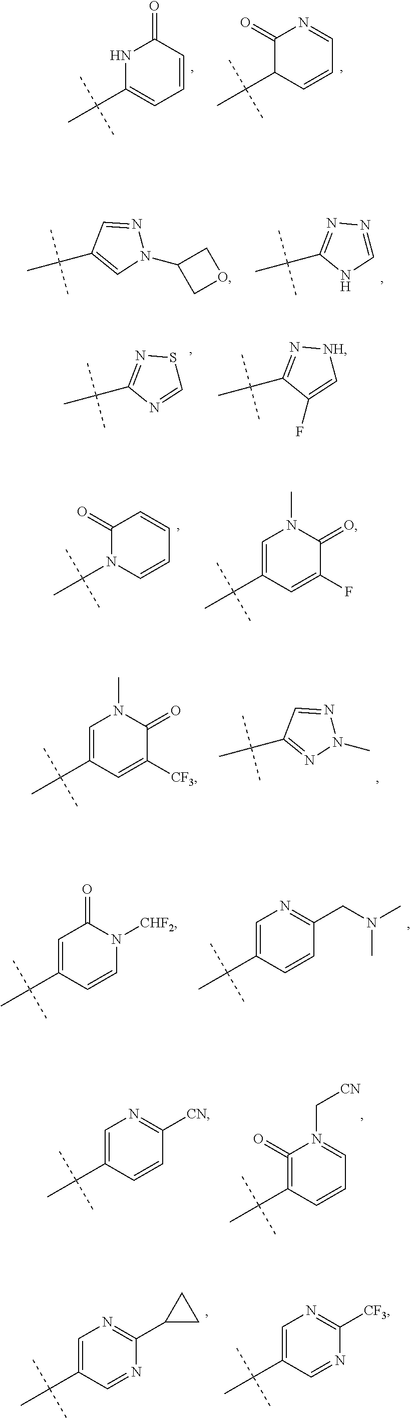

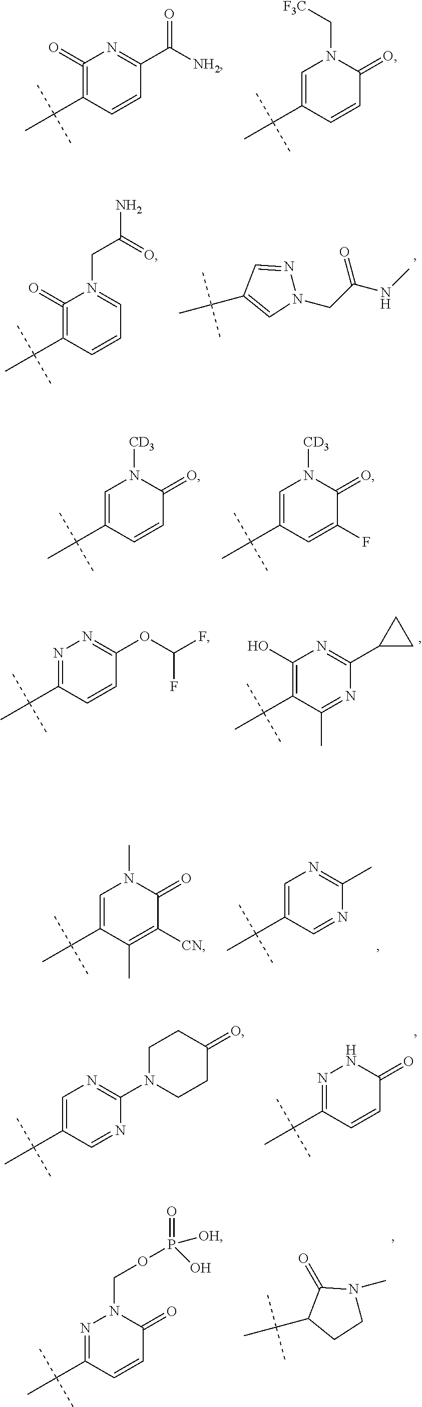

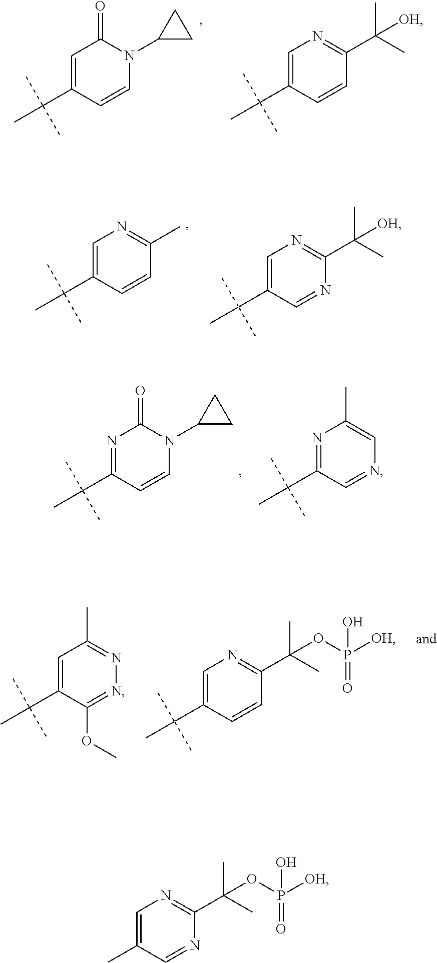

ii) a 9- or 10-membered N-containing bicyclic heterocycle selected from:

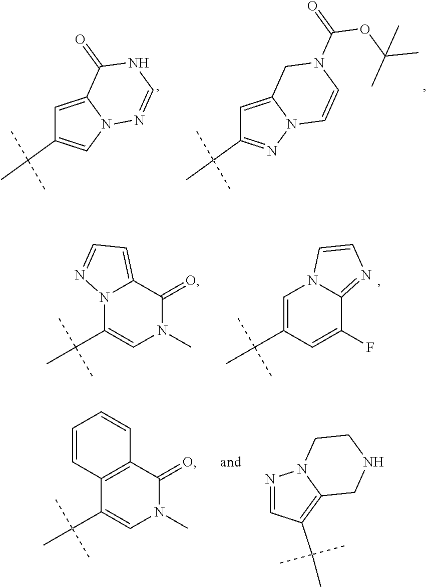

iii) cyclopropyl, cyclobutyl,
iv) Br,
v) C(O)OCH₃,
vi) P(O)(CH₃)₂, or

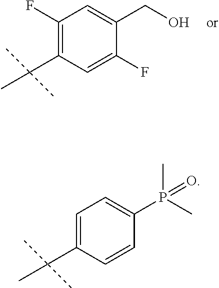

vii) In a particular embodiment, R⁹ is

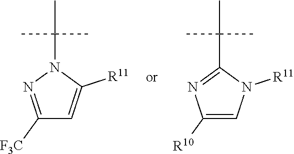

and R¹¹ is selected from the groups set out above.

In a further embodiment, R⁹ is:

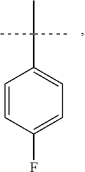

or
a 5- or 6-membered N-containing heteroaryl selected from:

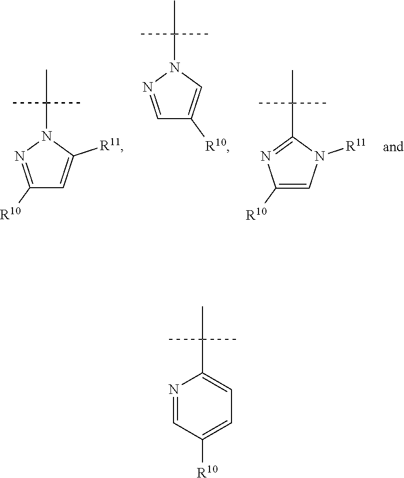

wherein R¹⁰ is CF₃, CF₂H or F, and R¹¹ is:
i) a 5- or 6-membered heterocycle selected from:

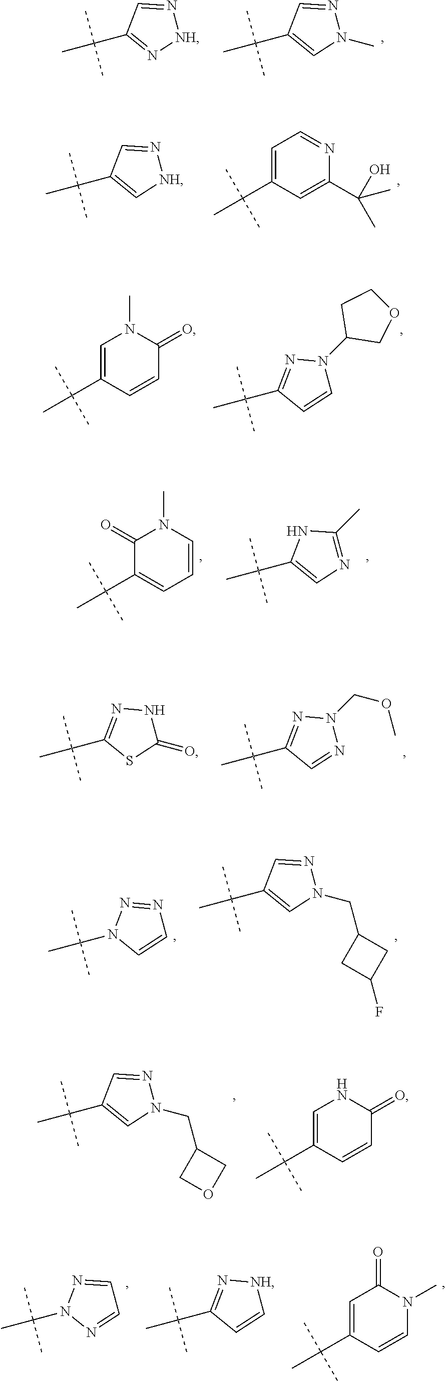

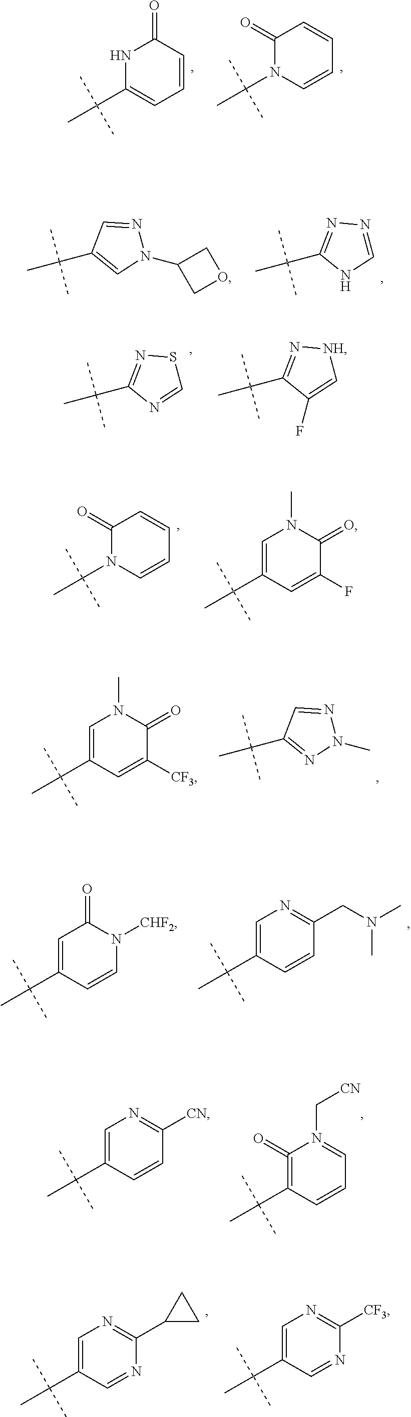

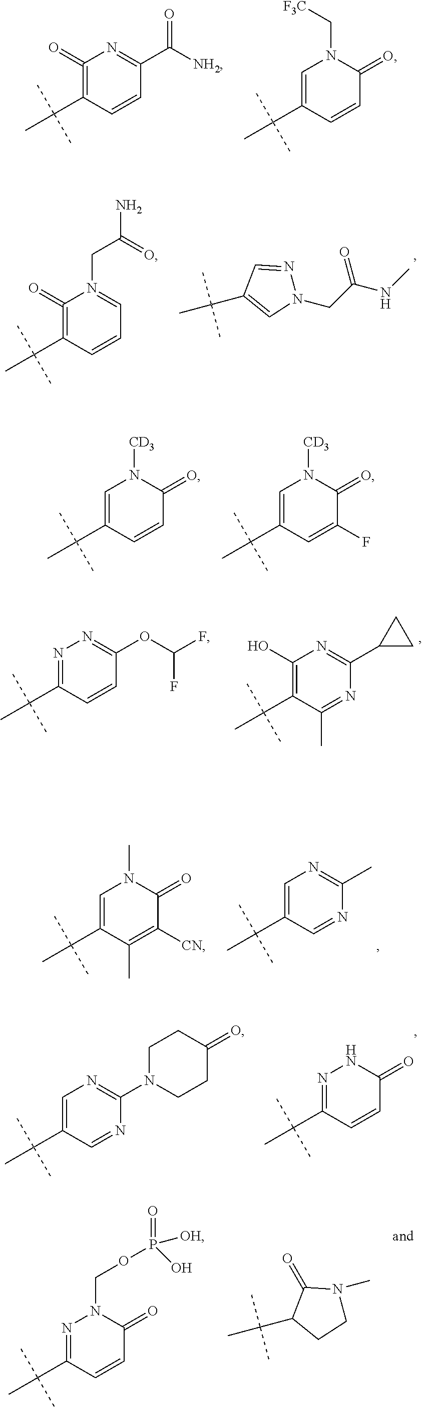

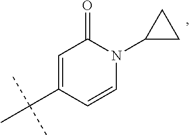

ii) a 9- or 10-membered N-containing bicyclic heterocycle selected from:

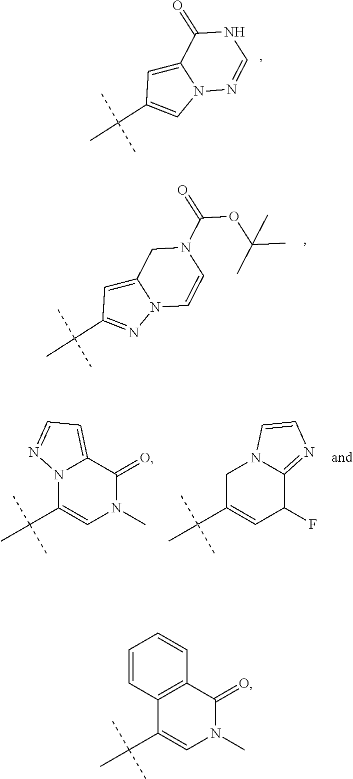

iii) cyclopropyl, cyclobutyl,
iv) Br,
v) C(O)OCH₃, or
vi)

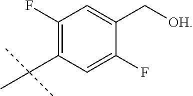

In a particular embodiment, R⁹ is

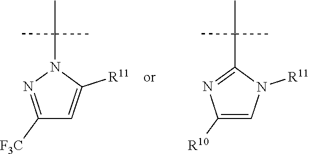

and R¹¹ is selected from the groups set out above.

In a further embodiment, R⁹ is:

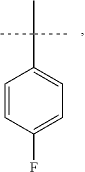

or
a 5- or 6-membered N-containing heteroaryl selected from:

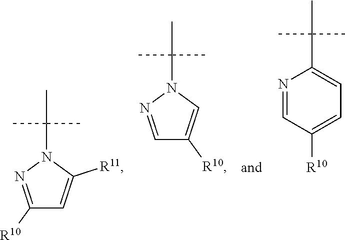

wherein R¹⁰ is CF₃, CF₂H or F, and R¹¹ is:
i) a 5- or 6-membered heterocycle selected from:

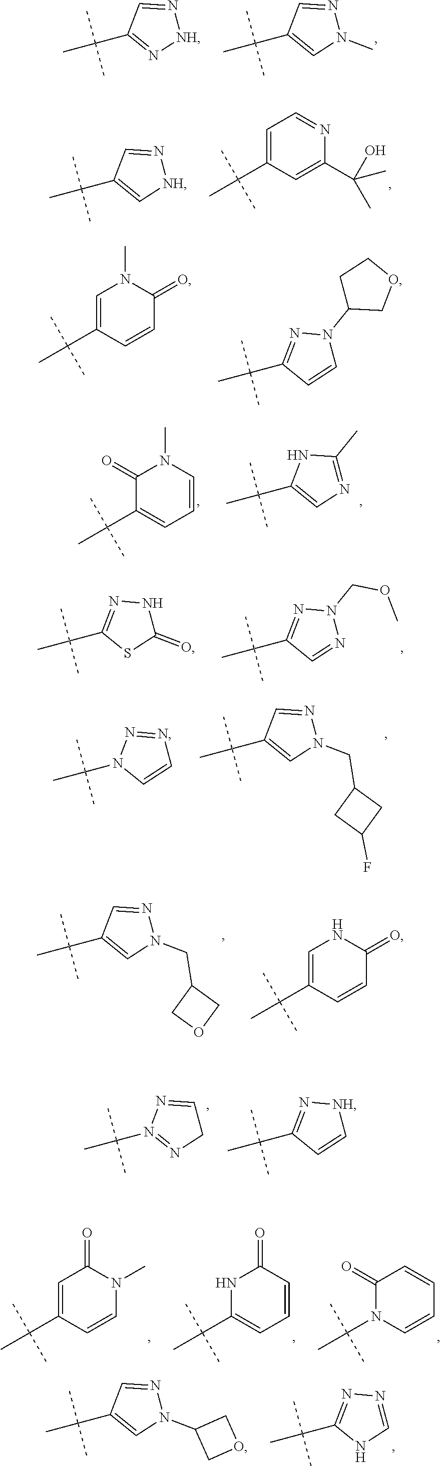

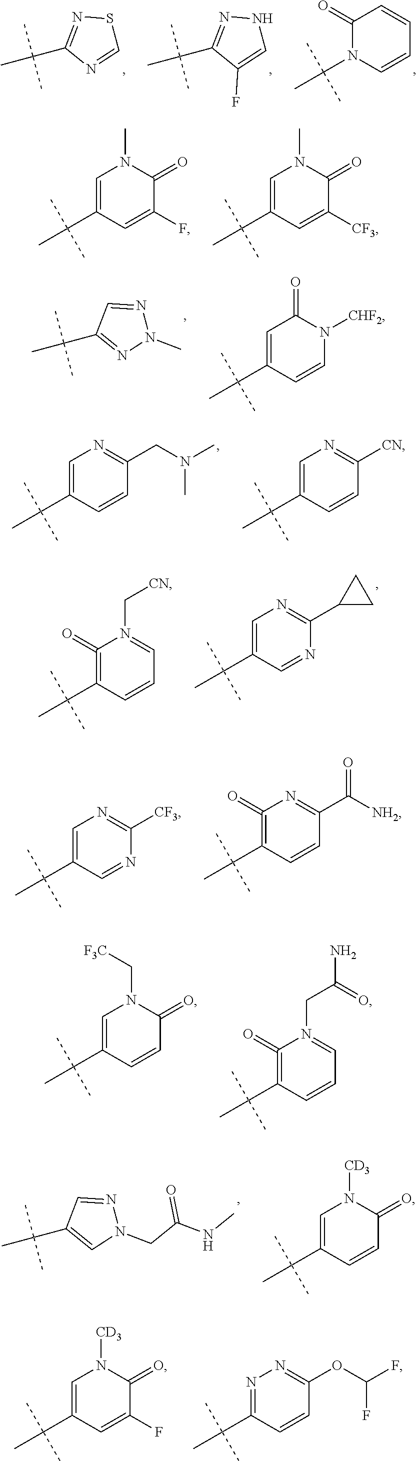

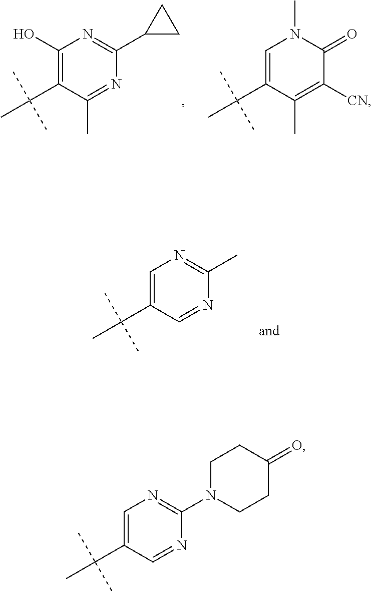

ii) a 9- or 10-membered N-containing bicyclic heterocycle selected from:

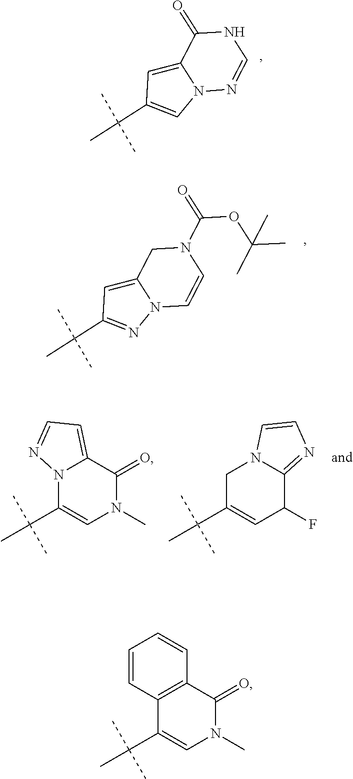

iii) cyclopropyl, cyclobutyl,
iv) Br,
v) C(O)OCH₃, or
vi)

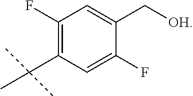

In a particular embodiment, R⁹ is

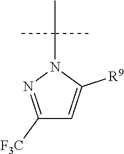

and R¹¹ is selected from the groups set out above.

In an alternate embodiment, R⁹ is:

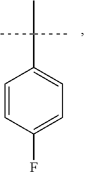

or
a 5- or 6-membered N-containing heteroaryl selected from:

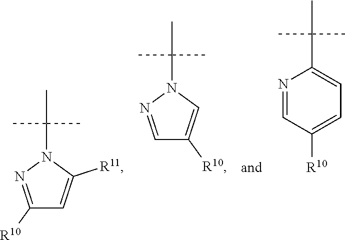

wherein R¹⁰ is CF₃, CF₂H or F, and R¹¹ is:
i) a 5- or 6-membered heterocycle selected from:

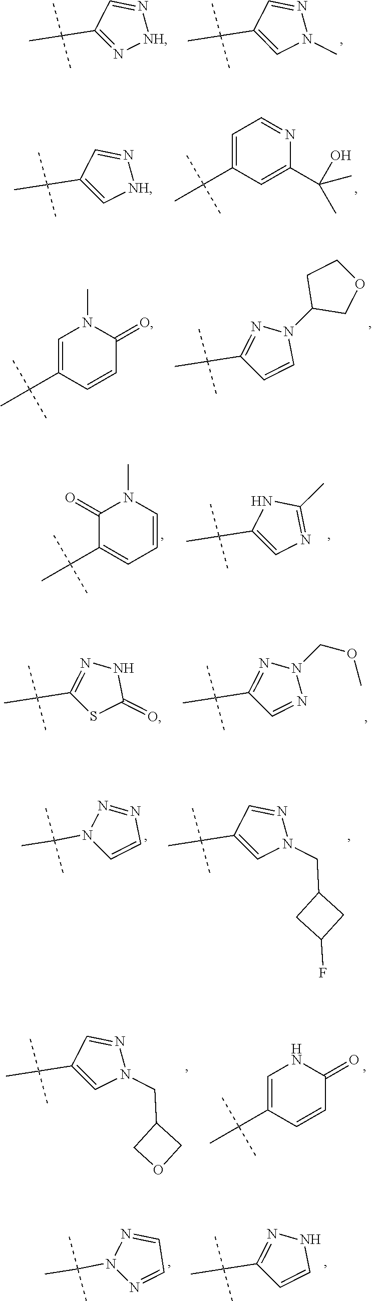

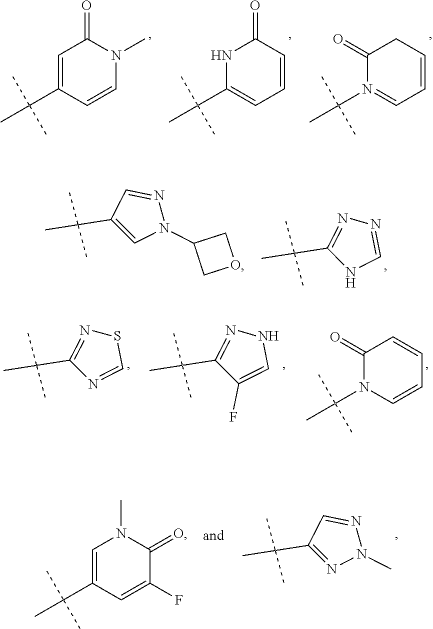

ii) a 9-membered N-containing bicyclic heterocycle selected from:

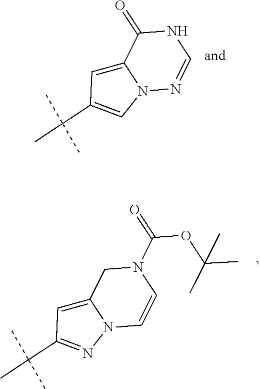

iii) cyclopropyl, cyclobutyl,
iv) Br, or
v) C(O)OCH₃.

In a further embodiment, R⁹ is:
a 5- or 6-membered N-containing heteroaryl selected from:

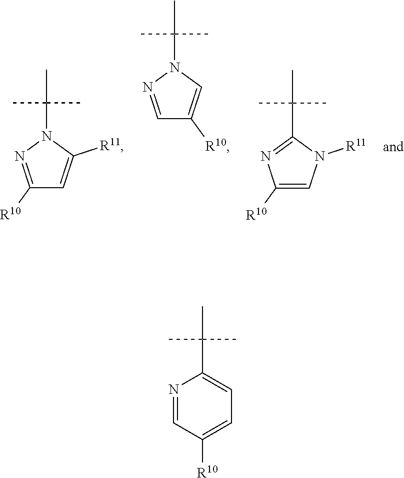

wherein R¹⁰ is CF₃, CF₂H or F, and R¹¹ is:
i) a 5- or 6-membered heterocycle selected from:

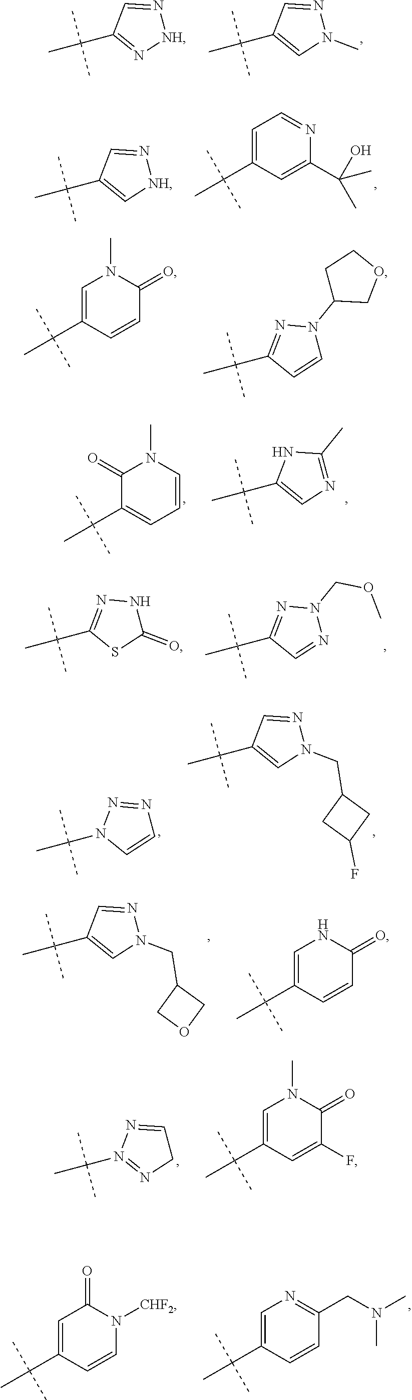

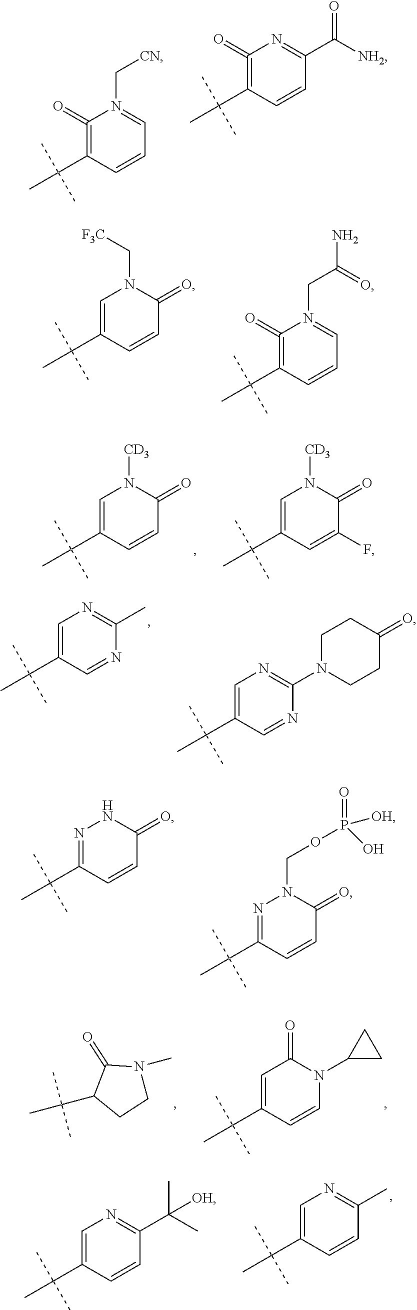

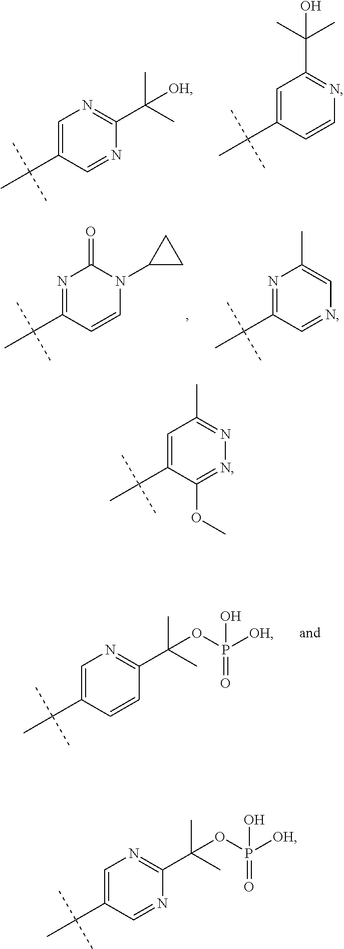

ii) a 9- or 10-membered N-containing bicyclic heterocycle selected from:

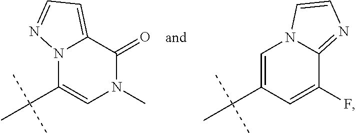

iii) cyclopropyl,
iv) C(O)OCH₃,
v) P(OXCH3)₂, or
vii)

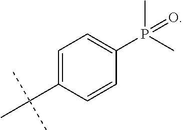

In a particular embodiment, R⁹ is

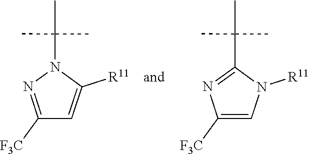

and R¹¹ is selected from the groups set out above.

In a further embodiment, R⁹ is:
a 5- or 6-membered N-containing heteroaryl selected from:

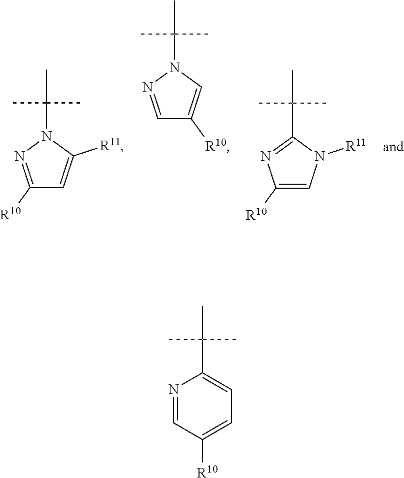

wherein R¹⁰ is CF₃, CF₂H or F, and R¹¹ is:
i) a 5- or 6-membered heterocycle selected from:

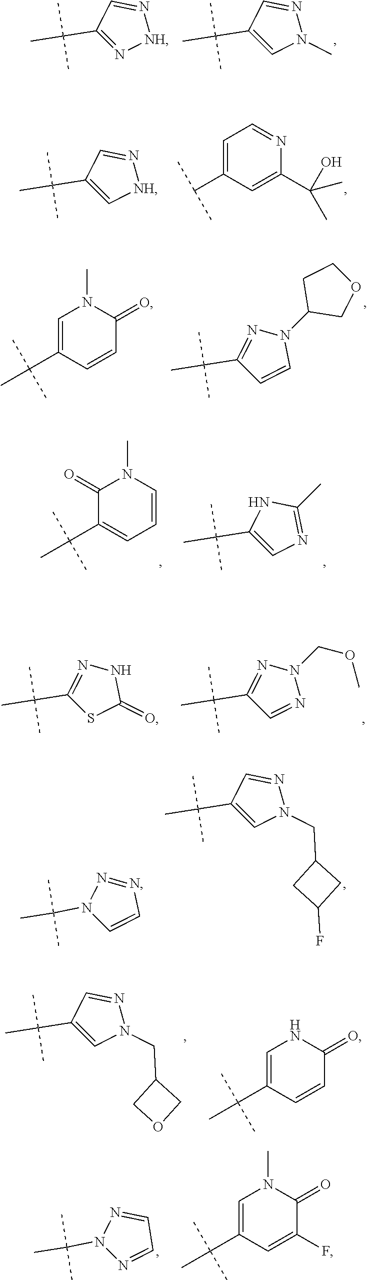

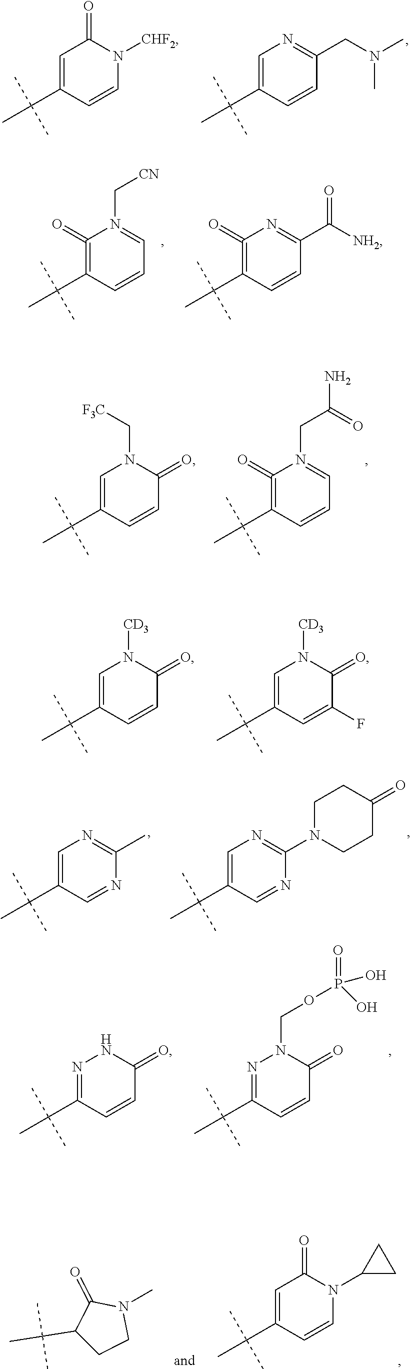

ii) a 9- or 10-membered N-containing bicyclic heterocycle selected from:

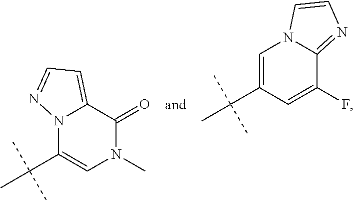

iii) cyclopropyl, or
viii) C(O)OCH₃. In a particular embodiment, R⁹ is

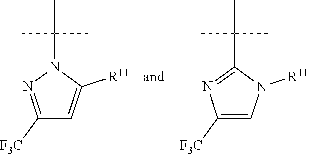

and
R¹¹ is selected from the groups set out above.

In a further embodiment, R⁹ is:
a 5- or 6-membered N-containing heteroaryl selected from:

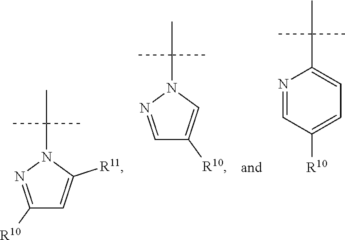

wherein R¹⁰ is CF₃, CF₂H or F, and R¹¹ is:
i) a 5- or 6-membered heterocycle selected from:

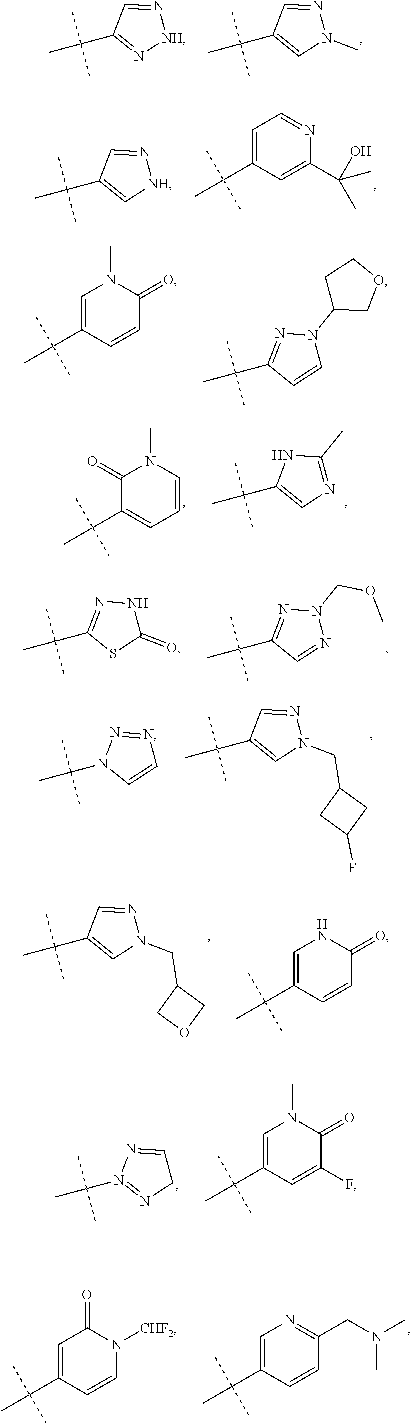

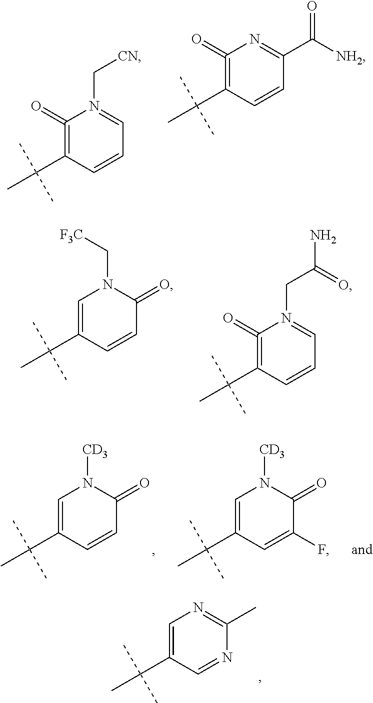

ii) a 9- or 10-membered N-containing bicyclic heterocycle selected from:

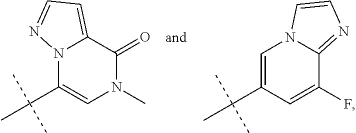

iii) cyclopropyl, or
vii) C(O)OCH₃. In a particular embodiment, R⁹ is

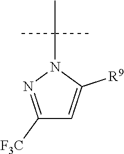

and R¹¹ is selected from the groups set out above.

In an alternate embodiment, R⁹ is:
a 5- or 6-membered N-containing heteroaryl selected from:

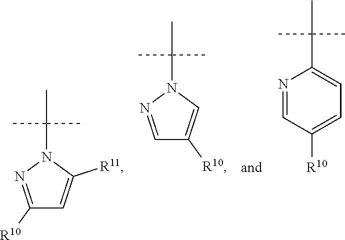

wherein R¹⁰ is CF₃ or F, and R¹¹ is:
i) a 5- or 6-membered heterocycle selected from:

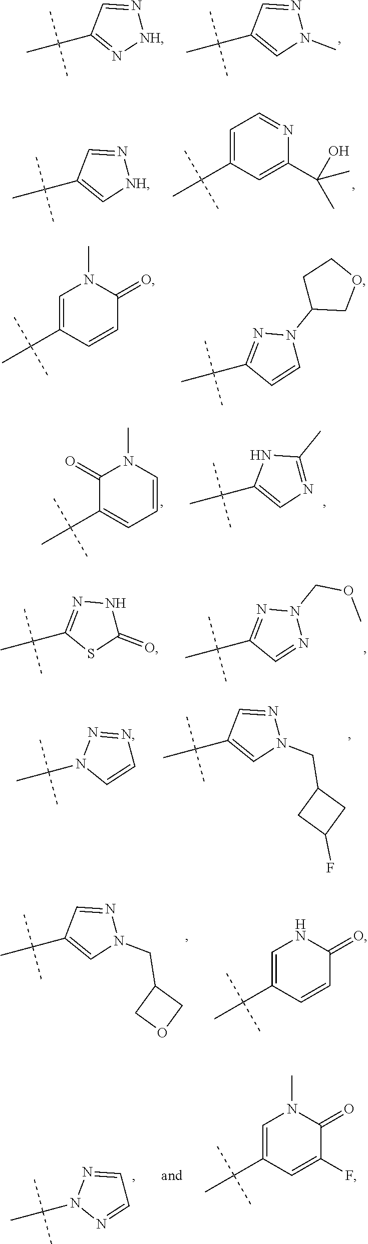

ii) cyclopropyl, or
iii) C(O)OCH₃.

In a particular embodiment, R⁹ is:

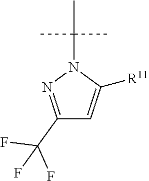

wherein R¹¹ is 5- or 6-membered heterocycle selected from:

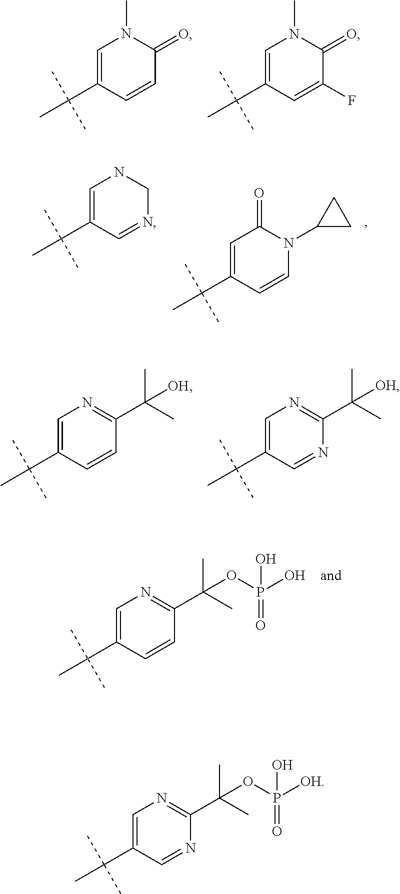

In a particular embodiment, R⁹ is:

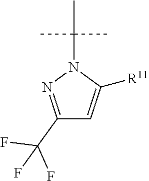

wherein R¹¹ is 5- or 6-membered heterocycle selected from:

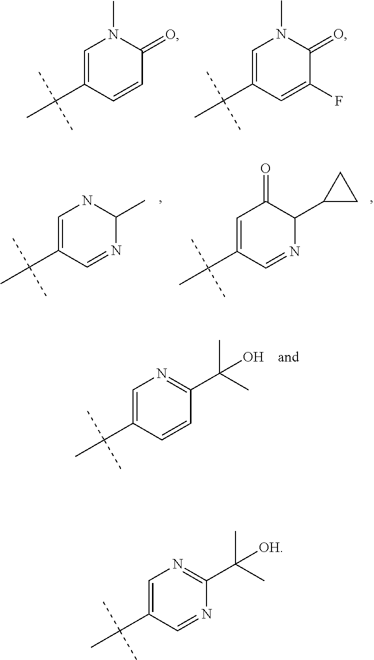

In an alternate embodiment, R⁹ is:

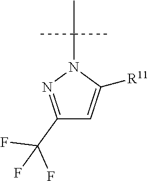

wherein R¹¹ is 5- or 6-membered heterocycle selected from:

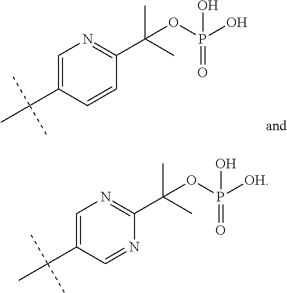

In a further embodiment, R⁹ is selected from:

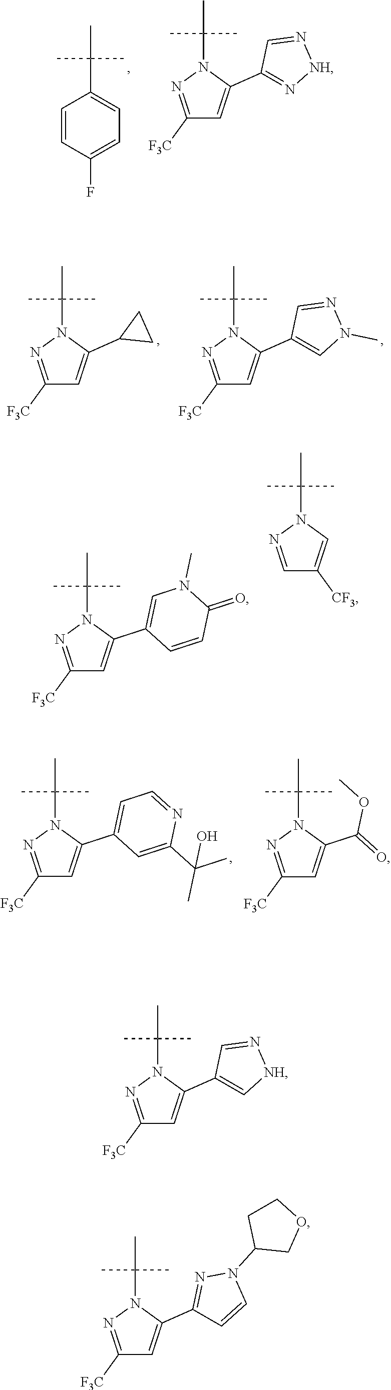

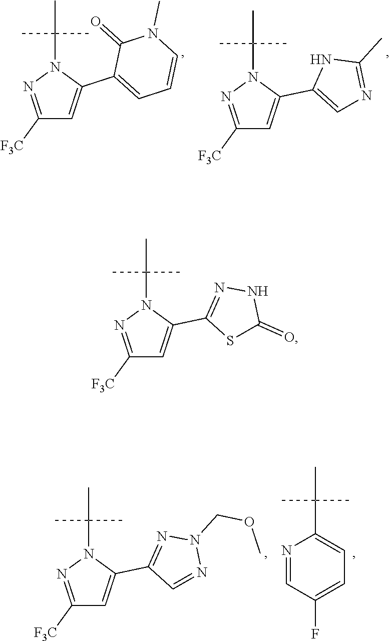

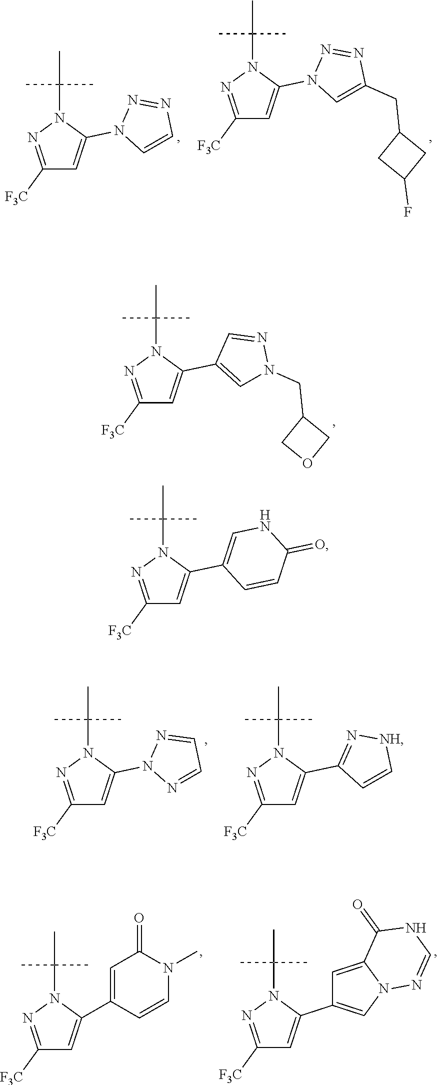

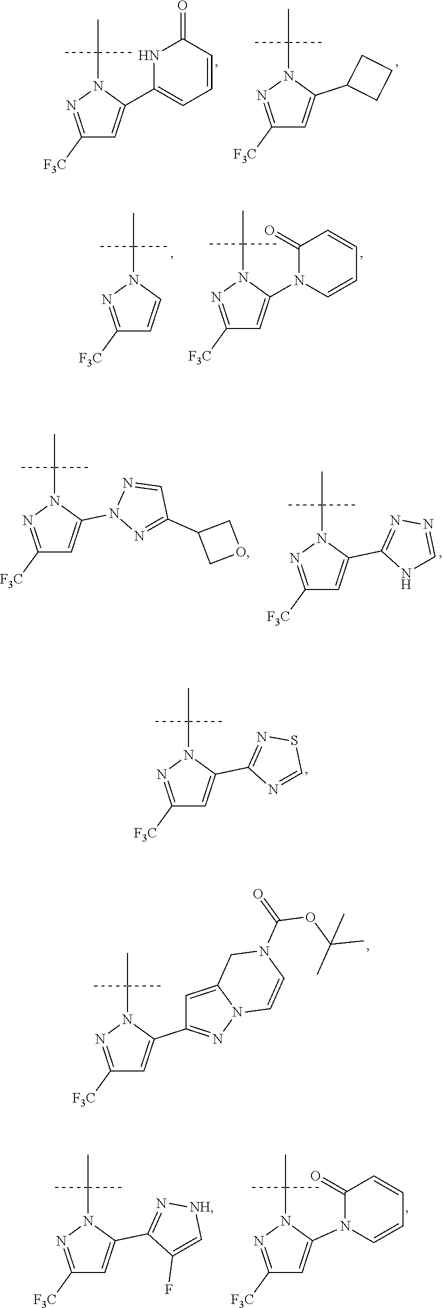

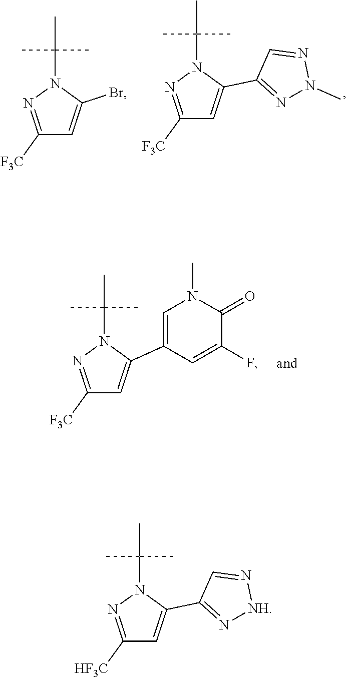

In a further embodiment, R⁹ is selected from:

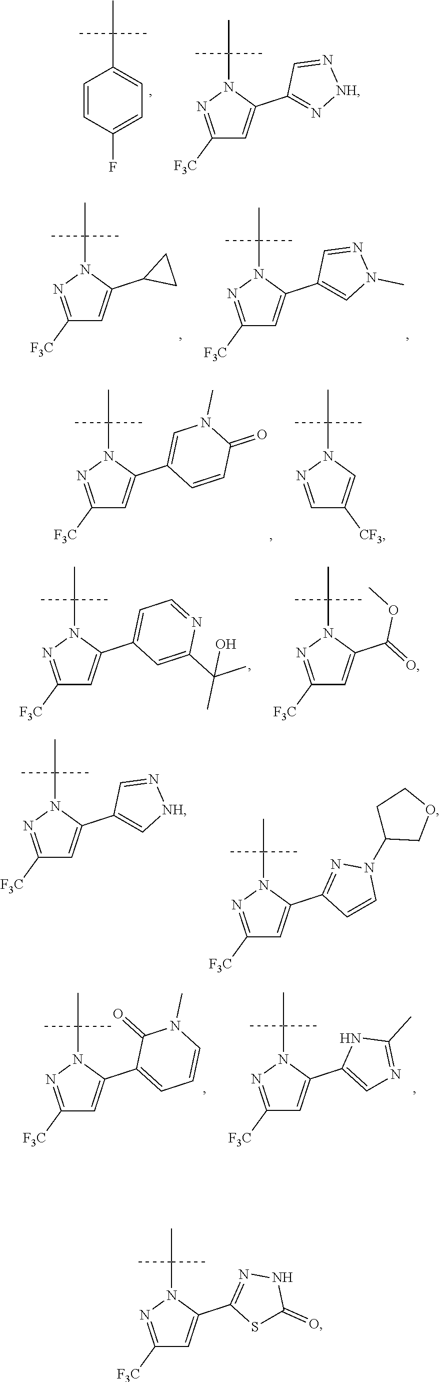

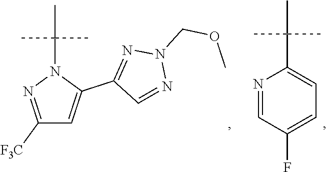

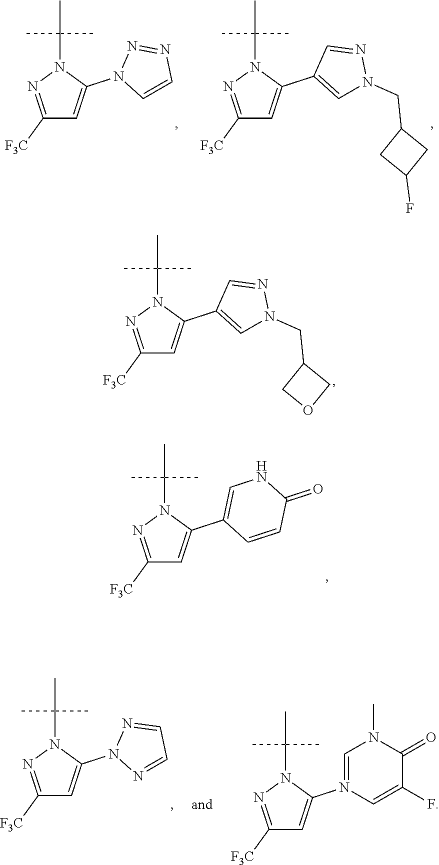

In a specific embodiment, R⁹ is selected from:

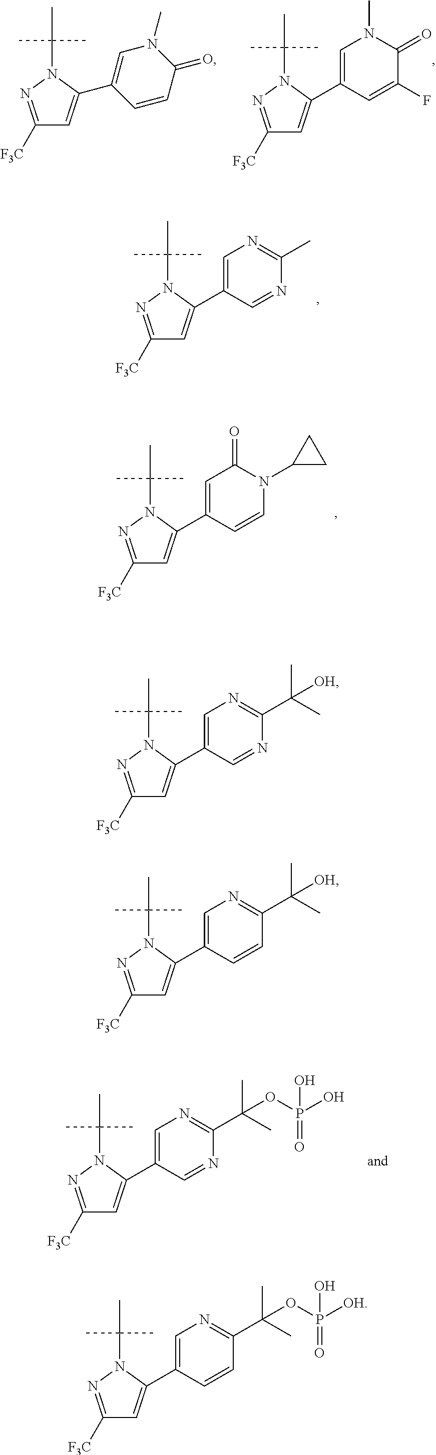

In a specific embodiment, R⁹ is selected from:

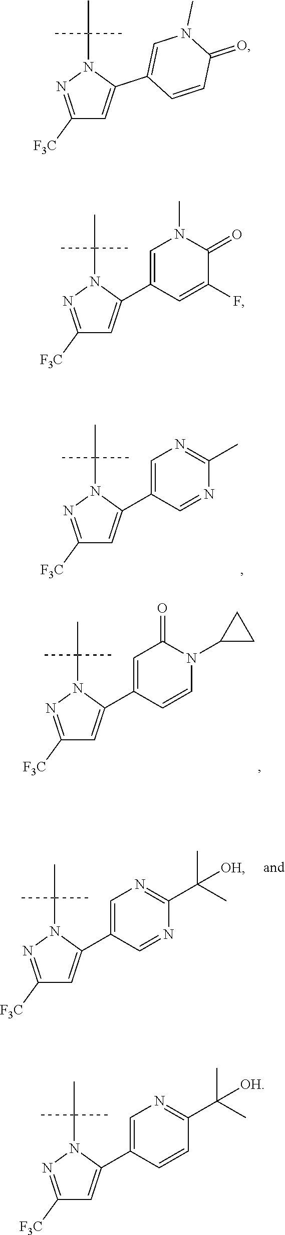

In an alternate specific embodiment, R⁹ is selected from:

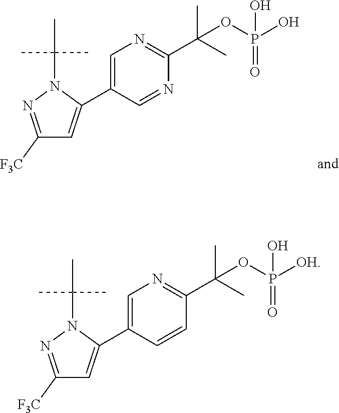

In a specific embodiment, R⁹ is selected from:

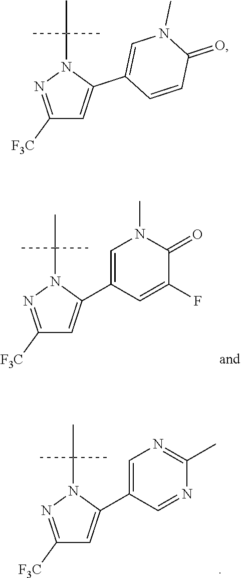

In one embodiment, the compound is selected from:

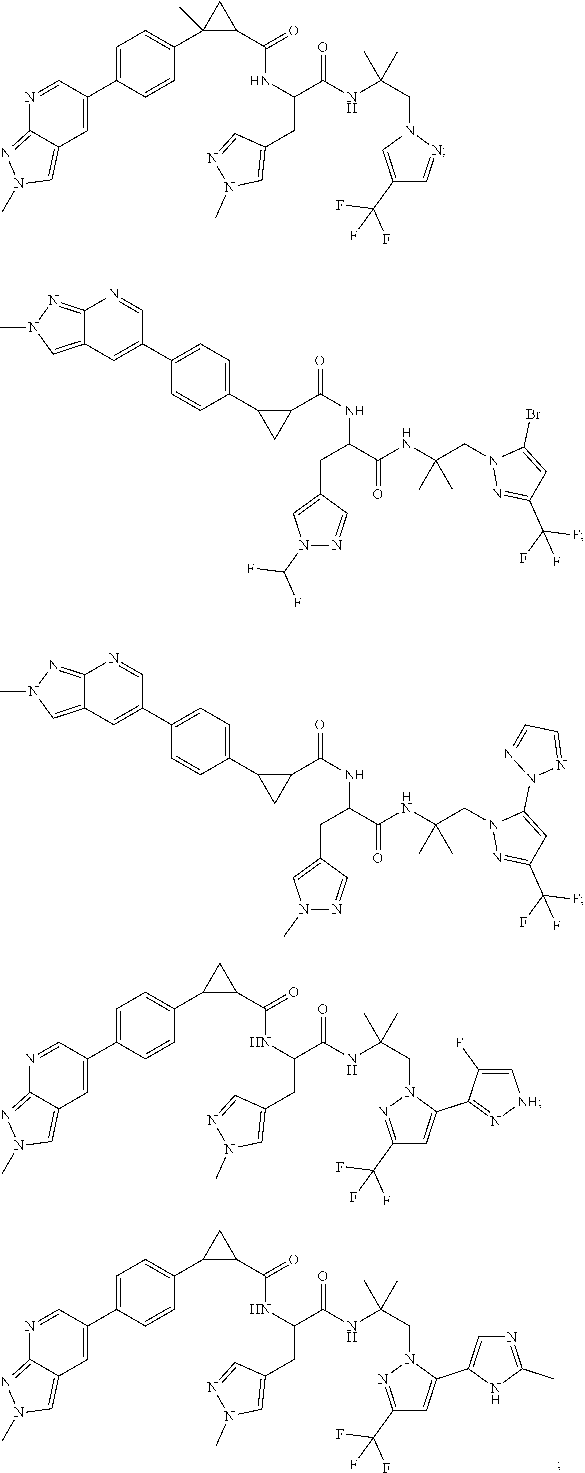

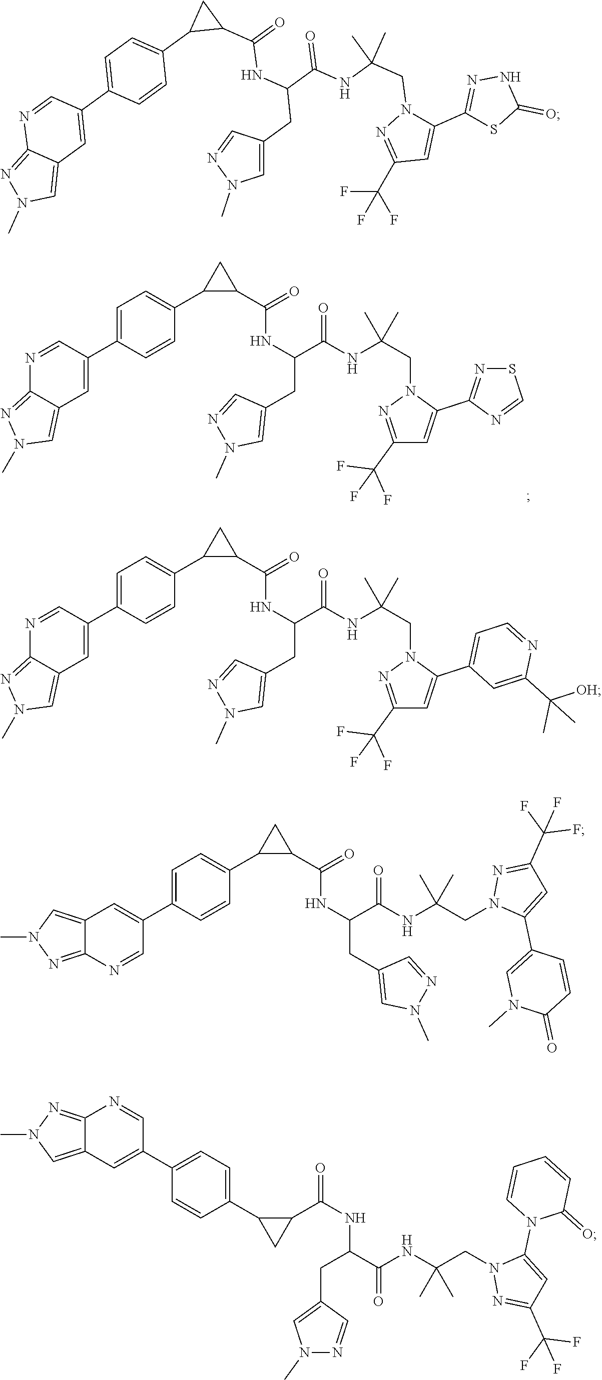

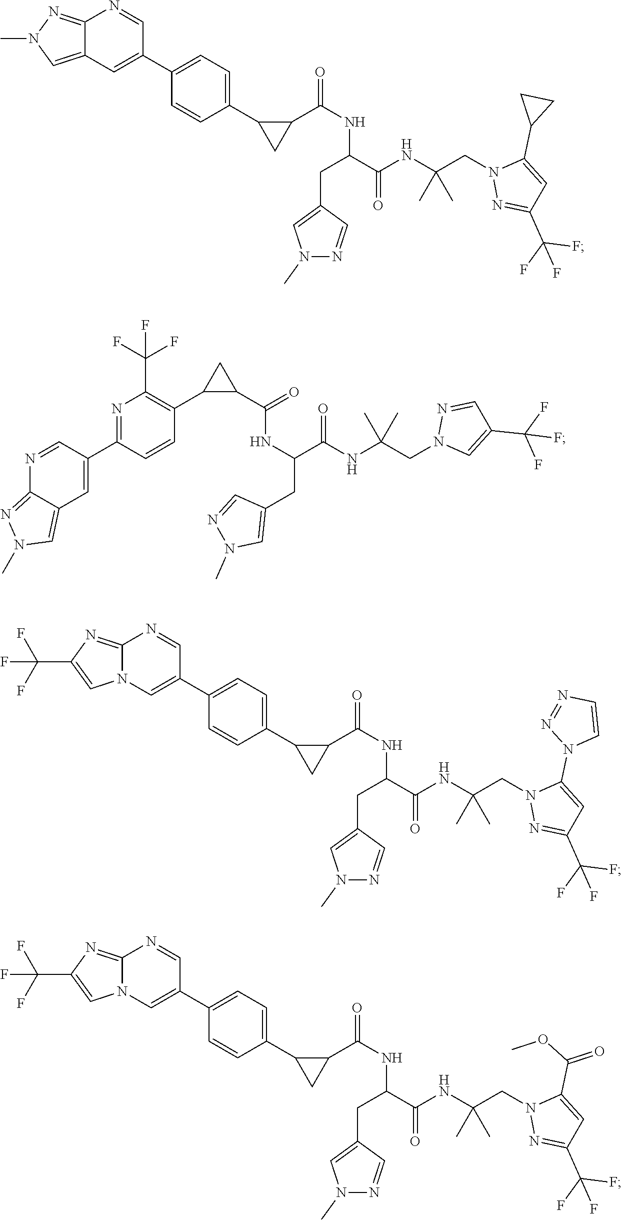 [SMILES: Cn1cc(CC(NC(=O)C2CC2c2ccc(-c3cnc4nn(C)cc4c3)cc2)C(=O)NC(C)(C)Cn2nc(C(F)(F)F)cc2C2CC2)cn1 ; Cn1cc(CC(NC(=O)C2CC2c2ccc(-c3cnc4nn(C)cc4c3)nc2C(F)(F)F)C(=O)NC(C)(C)Cn2cc(C(F)(F)F)cn2)cn1 ; Cn1cc(CC(NC(=O)C2CC2c2ccc(-c3cnc4nc(C(F)(F)F)cn4c3)cc2)C(=O)NC(C)(C)Cn2nc(C(F)(F)F)cc2-n2ccnn2)cn1 ; COC(=O)c1cc(C(F)(F)F)nn1CC(C)(C)NC(=O)C(Cc1cnn(C)c1)NC(=O)C1CC1c1ccc(-c2cnc3nc(C(F)(F)F)cn3c2)cc1]

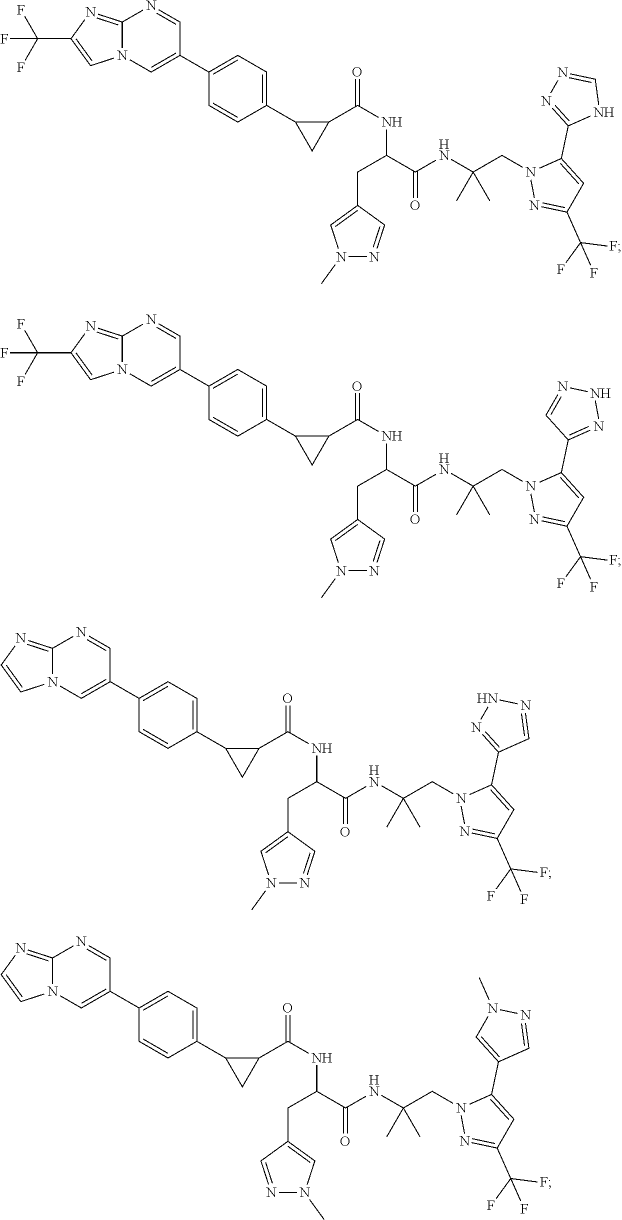 [SMILES: Cn1cc(CC(NC(=O)C2CC2c2ccc(-c3cnc4nc(C(F)(F)F)cn4c3)cc2)C(=O)NC(C)(C)Cn2nc(C(F)(F)F)cc2-c2nnc[nH]2)cn1 ; Cn1cc(CC(NC(=O)C2CC2c2ccc(-c3cnc4nc(C(F)(F)F)cn4c3)cc2)C(=O)NC(C)(C)Cn2nc(C(F)(F)F)cc2-c2cn[nH]n2)cn1 ; Cn1cc(CC(NC(=O)C2CC2c2ccc(-c3cnc4nccn4c3)cc2)C(=O)NC(C)(C)Cn2nc(C(F)(F)F)cc2-c2cn[nH]n2)cn1 ; Cn1cc(CC(NC(=O)C2CC2c2ccc(-c3cnc4nccn4c3)cc2)C(=O)NC(C)(C)Cn2nc(C(F)(F)F)cc2-c2cnn(C)c2)cn1]

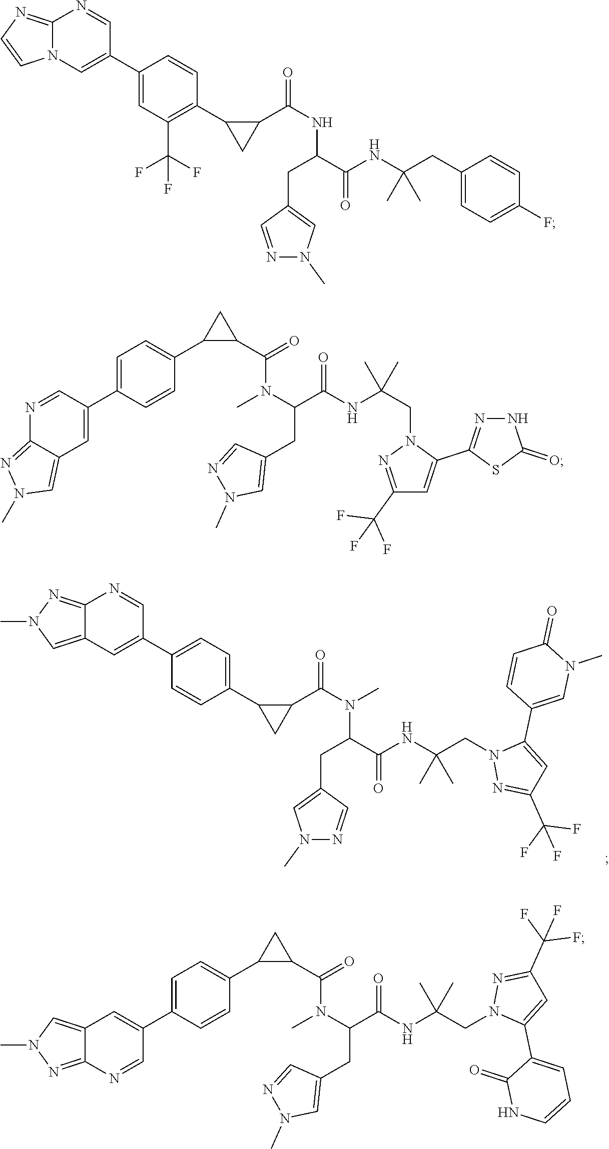

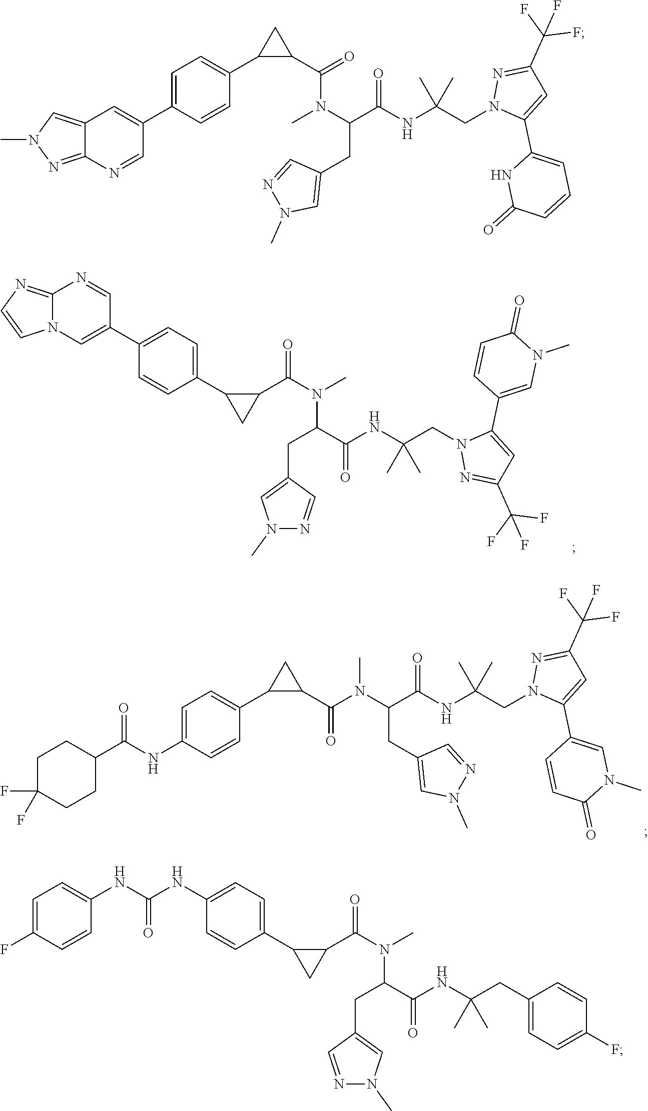 [SMILES: CN(C(=O)C1CC1c1ccc(-c2cnc3nn(C)cc3c2)cc1)C(Cc1cnn(C)c1)C(=O)NC(C)(C)Cn1nc(C(F)(F)F)cc1-c1cccc(=O)[nH]1 ; CN(C(=O)C1CC1c1ccc(-c2cnc3nccn3c2)cc1)C(Cc1cnn(C)c1)C(=O)NC(C)(C)Cn1nc(C(F)(F)F)cc1-c1ccc(=O)n(C)c1 ; CN(C(=O)C1CC1c1ccc(NC(=O)C2CCC(F)(F)CC2)cc1)C(Cc1cnn(C)c1)C(=O)NC(C)(C)Cn1nc(C(F)(F)F)cc1-c1ccc(=O)n(C)c1 ; CN(C(=O)C1CC1c1ccc(NC(=O)Nc2ccc(F)cc2)cc1)C(Cc1cnn(C)c1)C(=O)NC(C)(C)Cc1ccc(F)cc1]

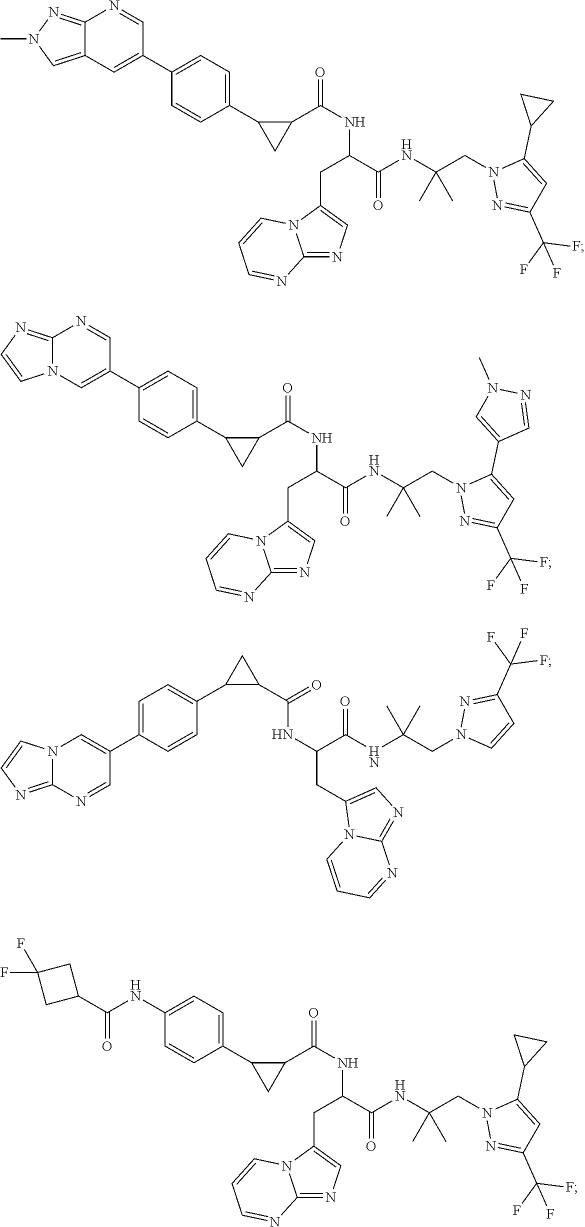

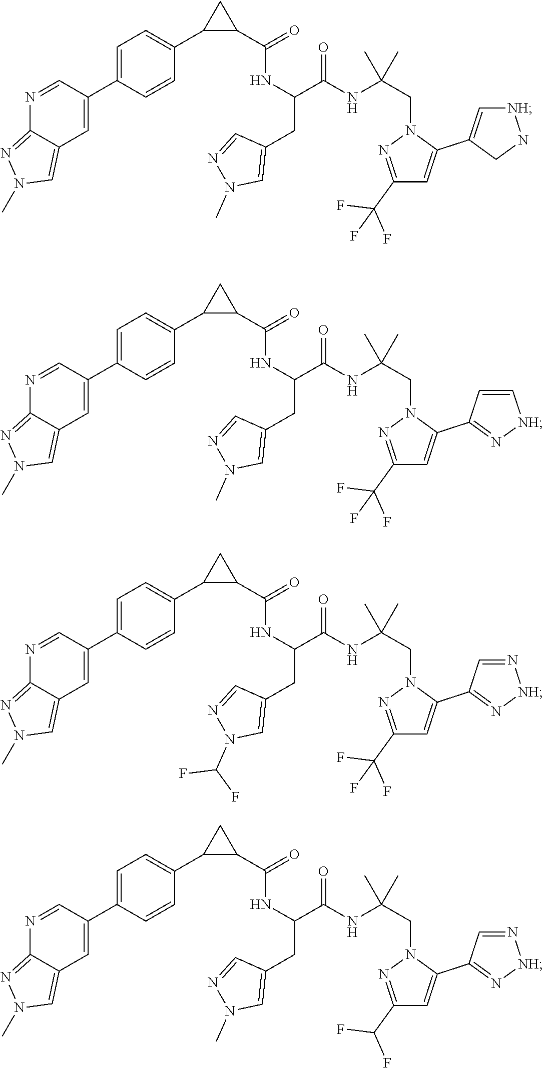

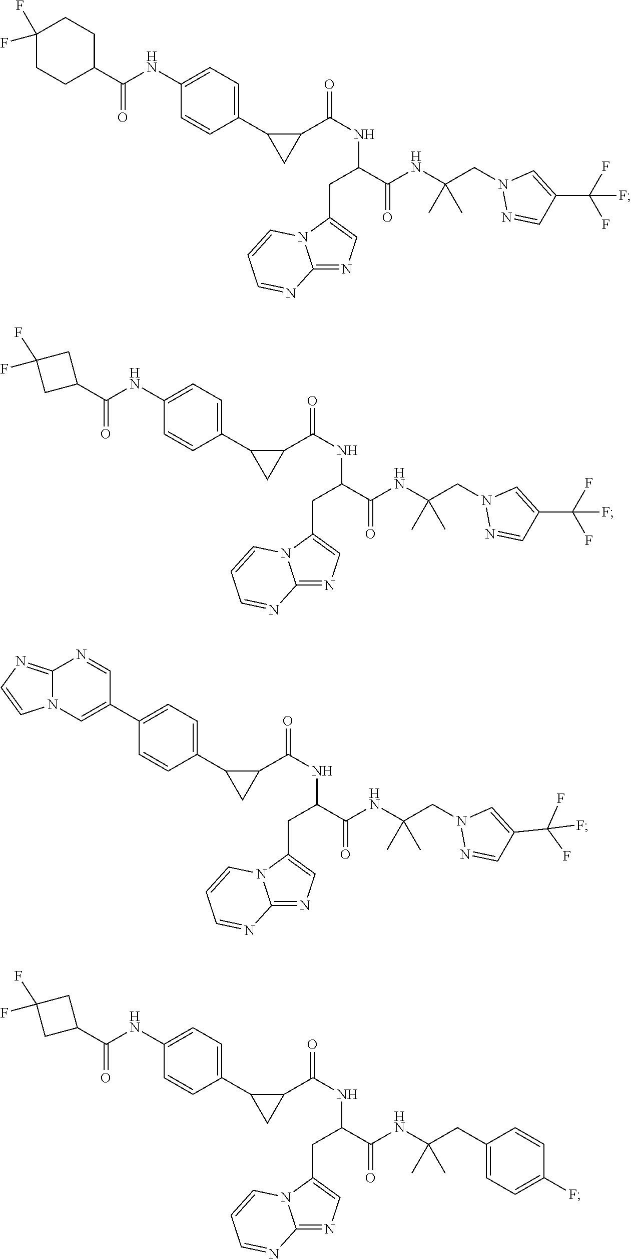 [SMILES: CC(C)(Cn1cc(C(F)(F)F)cn1)NC(=O)C(Cc1cnc2ncccn12)NC(=O)C1CC1c1ccc(NC(=O)C2CCC(F)(F)CC2)cc1 ; CC(C)(Cn1cc(C(F)(F)F)cn1)NC(=O)C(Cc1cnc2ncccn12)NC(=O)C1CC1c1ccc(NC(=O)C2CC(F)(F)C2)cc1 ; CC(C)(Cn1cc(C(F)(F)F)cn1)NC(=O)C(Cc1cnc2ncccn12)NC(=O)C1CC1c1ccc(-c2cnc3nccn3c2)cc1 ; CC(C)(Cc1ccc(F)cc1)NC(=O)C(Cc1cnc2ncccn12)NC(=O)C1CC1c1ccc(NC(=O)C2CC(F)(F)C2)cc1]

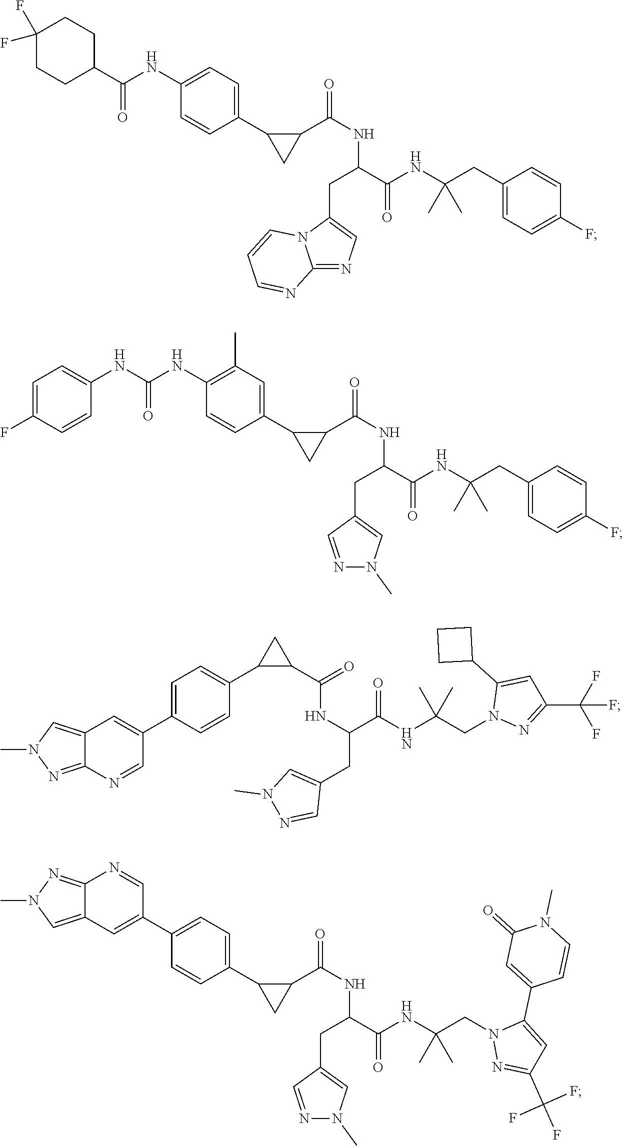 [SMILES: CC(C)(Cc1ccc(F)cc1)NC(=O)C(Cc1cnc2ncccn12)NC(=O)C1CC1c1ccc(NC(=O)C2CCC(F)(F)CC2)cc1 ; Cc1cc(C2CC2C(=O)NC(Cc2cnn(C)c2)C(=O)NC(C)(C)Cc2ccc(F)cc2)ccc1NC(=O)Nc1ccc(F)cc1 ; Cn1cc(CC(NC(=O)C2CC2c2ccc(-c3cnc4nn(C)cc4c3)cc2)C(=O)NC(C)(C)Cn2nc(C(F)(F)F)cc2C2CCC2)cn1 ; Cn1cc(CC(NC(=O)C2CC2c2ccc(-c3cnc4nn(C)cc4c3)cc2)C(=O)NC(C)(C)Cn2nc(C(F)(F)F)cc2-c2ccn(C)c(=O)c2)cn1]

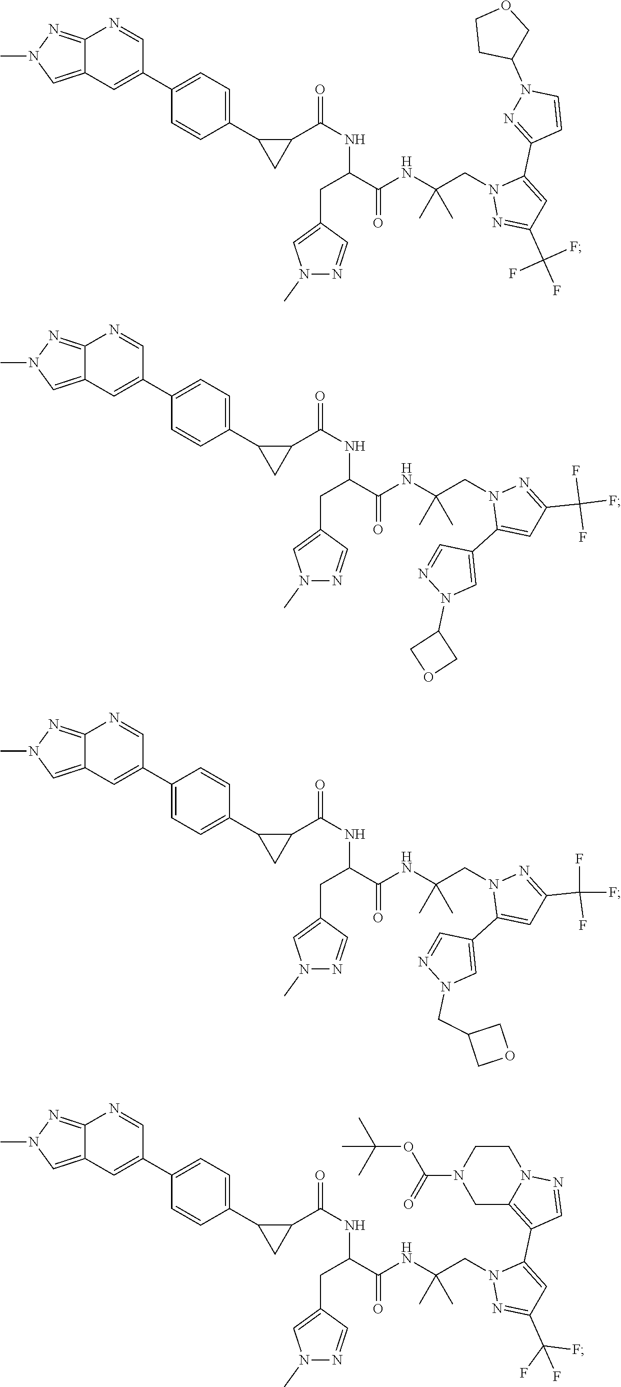 [SMILES: Cn1cc(CC(NC(=O)C2CC2c2ccc(-c3cnc4nn(C)cc4c3)cc2)C(=O)NC(C)(C)Cn2nc(C(F)(F)F)cc2-c2ccn(C3CCOC3)n2)cn1 ; Cn1cc(CC(NC(=O)C2CC2c2ccc(-c3cnc4nn(C)cc4c3)cc2)C(=O)NC(C)(C)Cn2nc(C(F)(F)F)cc2-c2cnn(C3COC3)c2)cn1 ; Cn1cc(CC(NC(=O)C2CC2c2ccc(-c3cnc4nn(C)cc4c3)cc2)C(=O)NC(C)(C)Cn2nc(C(F)(F)F)cc2-c2cnn(CC3COC3)c2)cn1 ; Cn1cc(CC(NC(=O)C2CC2c2ccc(-c3cnc4nn(C)cc4c3)cc2)C(=O)NC(C)(C)Cn2nc(C(F)(F)F)cc2-c2cnn3c2CN(C(=O)OC(C)(C)C)CC3)cn1]

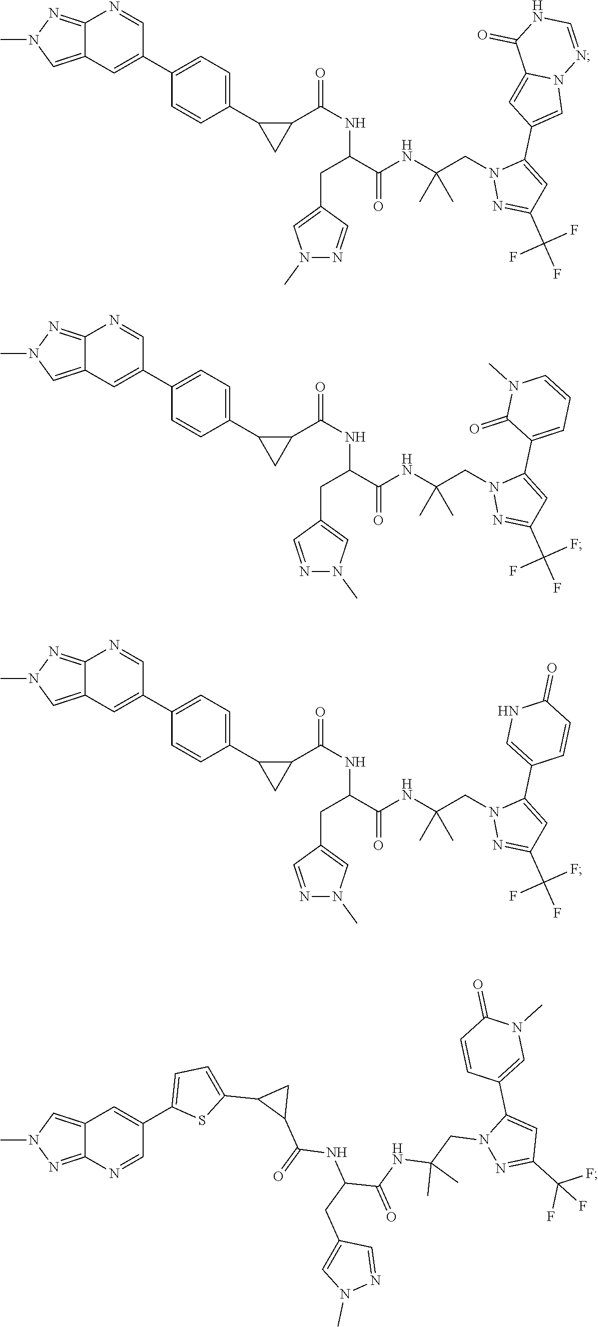 [SMILES: Cn1cc(CC(NC(=O)C2CC2c2ccc(-c3cnc4nn(C)cc4c3)cc2)C(=O)NC(C)(C)Cn2nc(C(F)(F)F)cc2-c2cc3c(=O)[nH]cnn3c2)cn1 ; Cn1cc(CC(NC(=O)C2CC2c2ccc(-c3cnc4nn(C)cc4c3)cc2)C(=O)NC(C)(C)Cn2nc(C(F)(F)F)cc2-c2cccn(C)c2=O)cn1 ; Cn1cc(CC(NC(=O)C2CC2c2ccc(-c3cnc4nn(C)cc4c3)cc2)C(=O)NC(C)(C)Cn2nc(C(F)(F)F)cc2-c2ccc(=O)[nH]c2)cn1 ; Cn1cc(CC(NC(=O)C2CC2c2ccc(-c3cnc4nn(C)cc4c3)s2)C(=O)NC(C)(C)Cn2nc(C(F)(F)F)cc2-c2ccc(=O)n(C)c2)cn1]

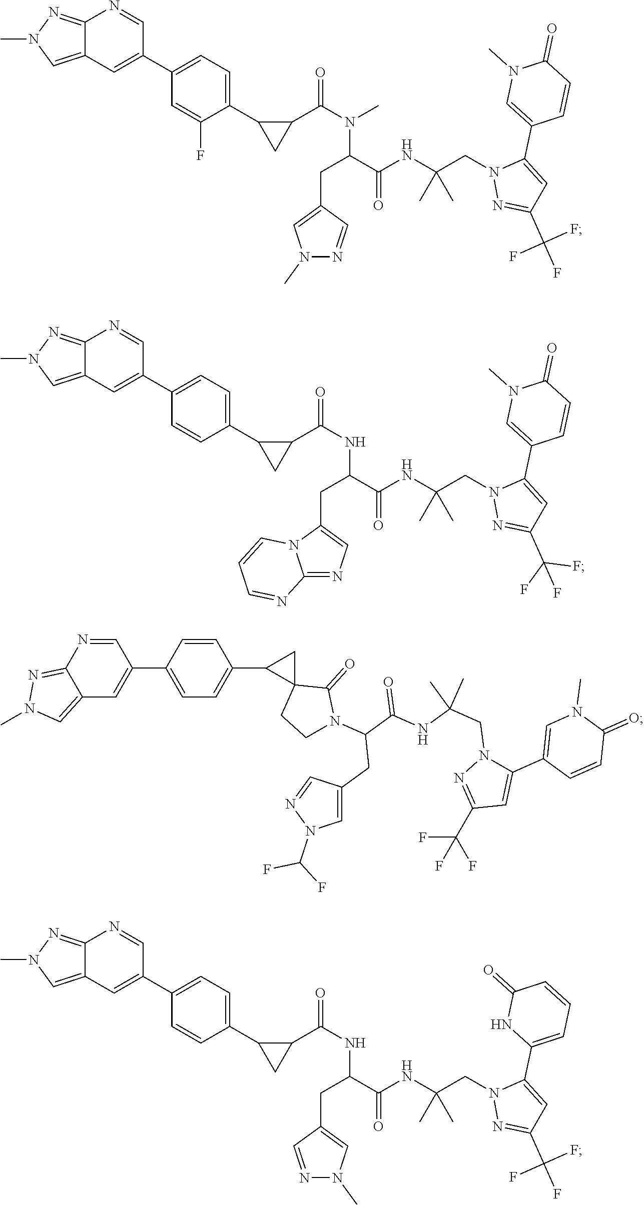 [SMILES: CN(C(=O)C1CC1c1ccc(-c2cnc3nn(C)cc3c2)cc1F)C(Cc1cnn(C)c1)C(=O)NC(C)(C)Cn1nc(C(F)(F)F)cc1-c1ccc(=O)n(C)c1 ; Cn1cc2cc(-c3ccc(C4CC4C(=O)NC(Cc4cnc5ncccn45)C(=O)NC(C)(C)Cn4nc(C(F)(F)F)cc4-c4ccc(=O)n(C)c4)cc3)cnc2n1 ; Cn1cc2cc(-c3ccc(C4CC45CCN(C(Cc4cnn(C(F)F)c4)C(=O)NC(C)(C)Cn4nc(C(F)(F)F)cc4-c4ccc(=O)n(C)c4)C5=O)cc3)cnc2n1 ; Cn1cc(CC(NC(=O)C2CC2c2ccc(-c3cnc4nn(C)cc4c3)cc2)C(=O)NC(C)(C)Cn2nc(C(F)(F)F)cc2-c2cccc(=O)[nH]2)cn1]

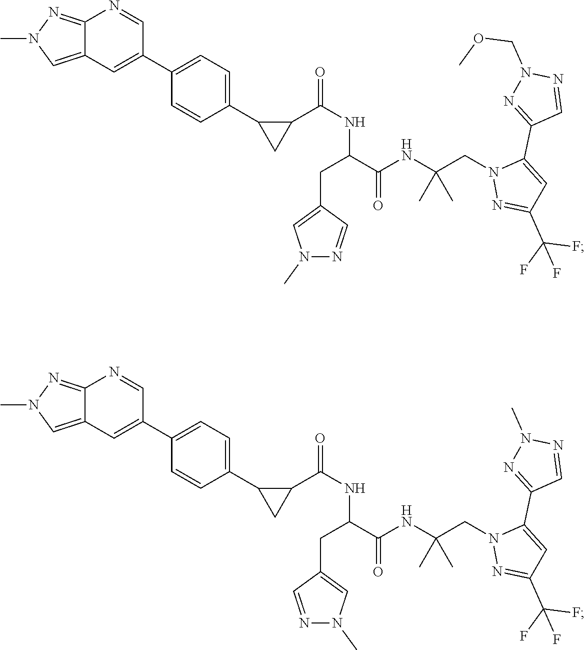 [SMILES: COCn1ncc(-c2cc(C(F)(F)F)nn2CC(C)(C)NC(=O)C(Cc2cnn(C)c2)NC(=O)C2CC2c2ccc(-c3cnc4nn(C)cc4c3)cc2)n1 ; Cn1cc(CC(NC(=O)C2CC2c2ccc(-c3cnc4nn(C)cc4c3)cc2)C(=O)NC(C)(C)Cn2nc(C(F)(F)F)cc2-c2cnn(C)n2)cn1]

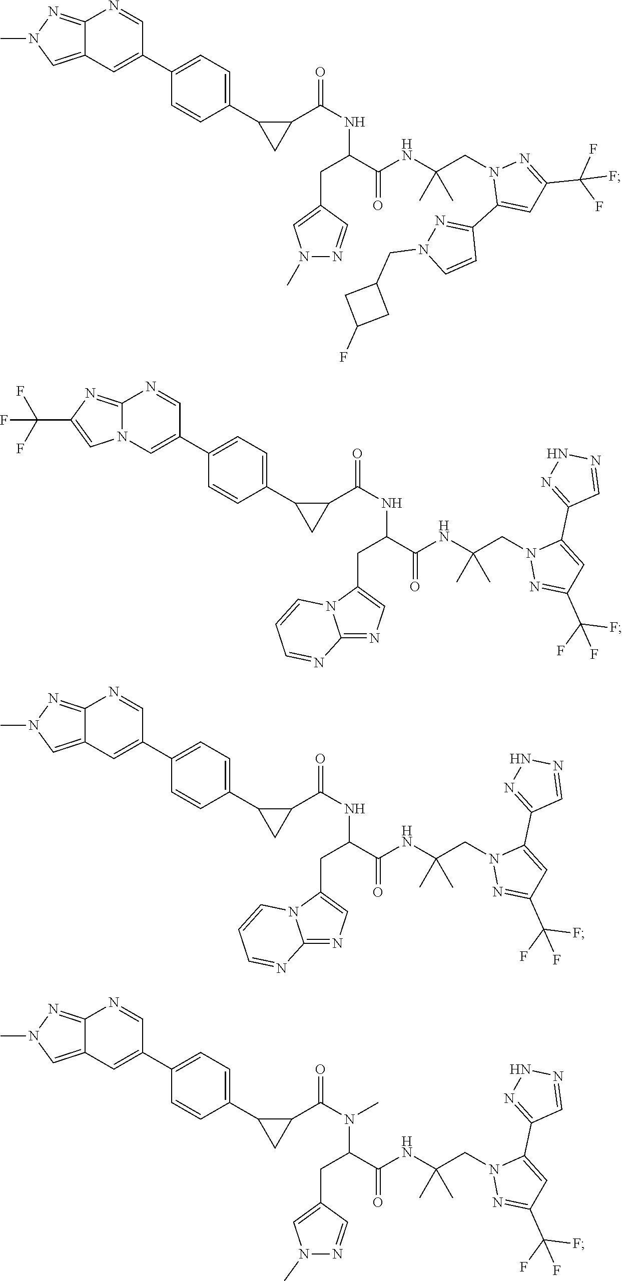 [SMILES: Cn1cc(CC(NC(=O)C2CC2c2ccc(-c3cnc4nn(C)cc4c3)cc2)C(=O)NC(C)(C)Cn2nc(C(F)(F)F)cc2-c2ccn(CC3CC(F)C3)n2)cn1 ; CC(C)(Cn1nc(C(F)(F)F)cc1-c1cn[nH]n1)NC(=O)C(Cc1cnc2ncccn12)NC(=O)C1CC1c1ccc(-c2cnc3nc(C(F)(F)F)cn3c2)cc1 ; Cn1cc2cc(-c3ccc(C4CC4C(=O)NC(Cc4cnc5ncccn45)C(=O)NC(C)(C)Cn4nc(C(F)(F)F)cc4-c4cn[nH]n4)cc3)cnc2n1 ; CN(C(=O)C1CC1c1ccc(-c2cnc3nn(C)cc3c2)cc1)C(Cc1cnn(C)c1)C(=O)NC(C)(C)Cn1nc(C(F)(F)F)cc1-c1cn[nH]n1]

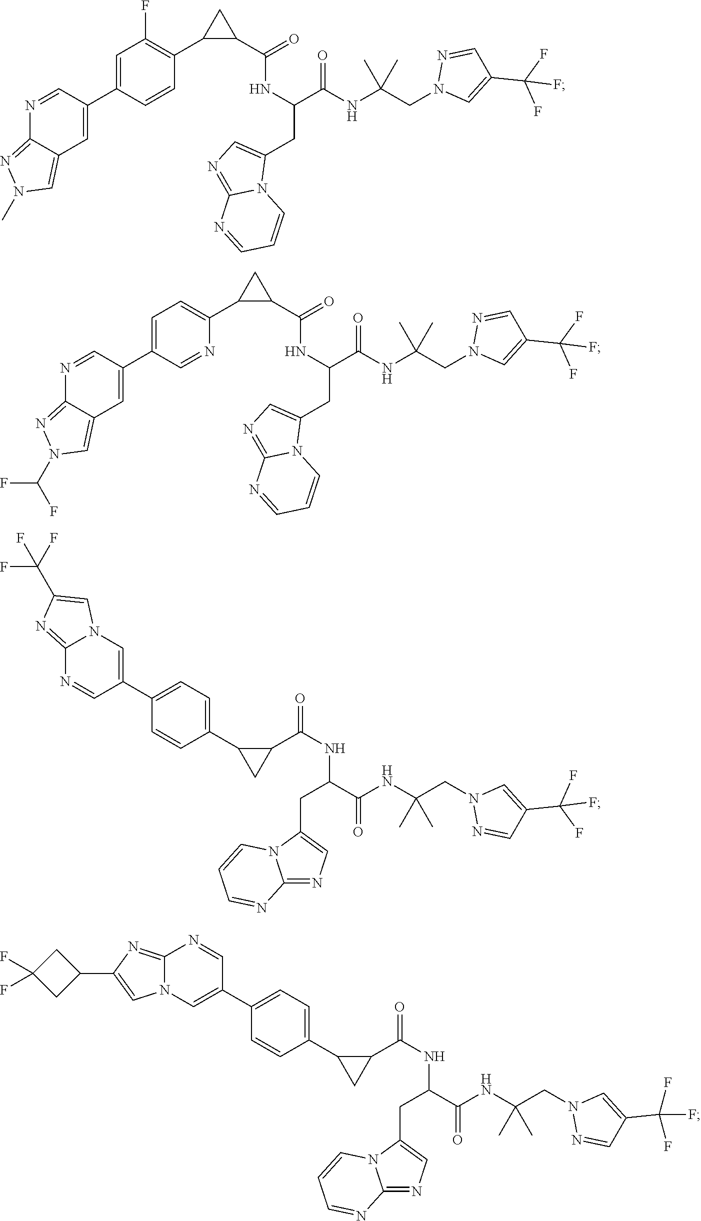

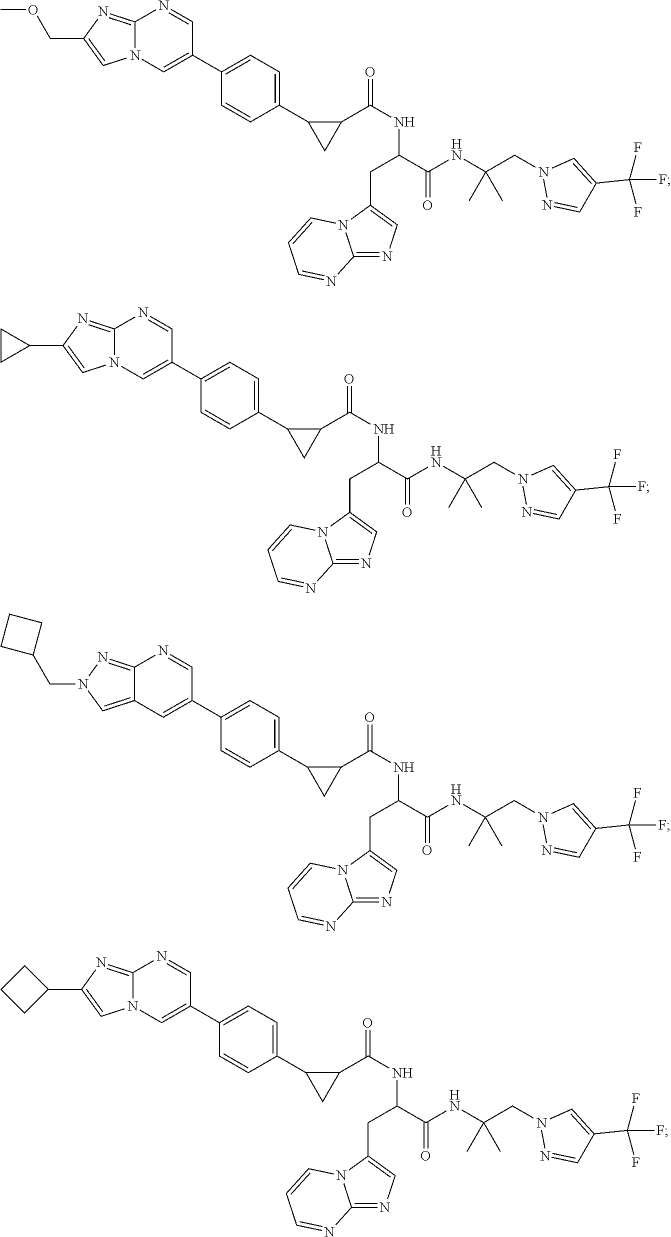 [SMILES: COCc1cn2cc(-c3ccc(C4CC4C(=O)NC(Cc4cnc5ncccn45)C(=O)NC(C)(C)Cn4cc(C(F)(F)F)cn4)cc3)cnc2n1 ; CC(C)(Cn1cc(C(F)(F)F)cn1)NC(=O)C(Cc1cnc2ncccn12)NC(=O)C1CC1c1ccc(-c2cnc3nc(C4CC4)cn3c2)cc1 ; CC(C)(Cn1cc(C(F)(F)F)cn1)NC(=O)C(Cc1cnc2ncccn12)NC(=O)C1CC1c1ccc(-c2cnc3nn(CC4CCC4)cc3c2)cc1 ; CC(C)(Cn1cc(C(F)(F)F)cn1)NC(=O)C(Cc1cnc2ncccn12)NC(=O)C1CC1c1ccc(-c2cnc3nc(C4CCC4)cn3c2)cc1]

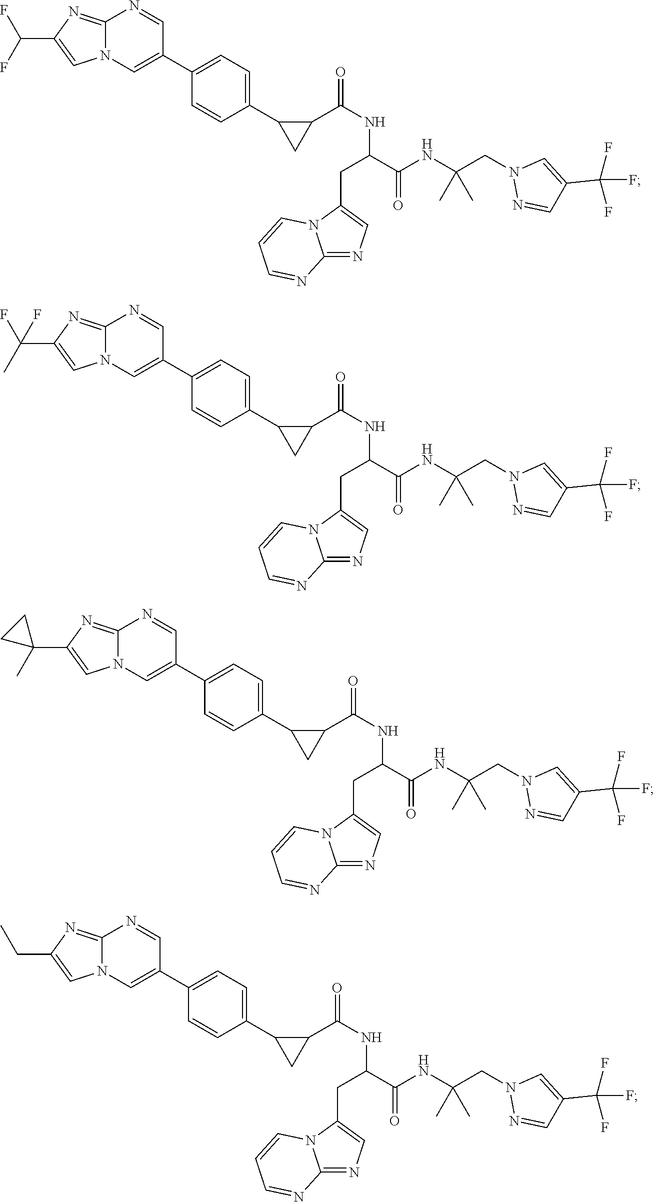 [SMILES: CC(C)(Cn1cc(C(F)(F)F)cn1)NC(=O)C(Cc1cnc2ncccn12)NC(=O)C1CC1c1ccc(-c2cnc3nc(C(F)F)cn3c2)cc1 ; CC(C)(Cn1cc(C(F)(F)F)cn1)NC(=O)C(Cc1cnc2ncccn12)NC(=O)C1CC1c1ccc(-c2cnc3nc(C(C)(F)F)cn3c2)cc1 ; CC(C)(Cn1cc(C(F)(F)F)cn1)NC(=O)C(Cc1cnc2ncccn12)NC(=O)C1CC1c1ccc(-c2cnc3nc(C4(C)CC4)cn3c2)cc1 ; CCc1cn2cc(-c3ccc(C4CC4C(=O)NC(Cc4cnc5ncccn45)C(=O)NC(C)(C)Cn4cc(C(F)(F)F)cn4)cc3)cnc2n1]

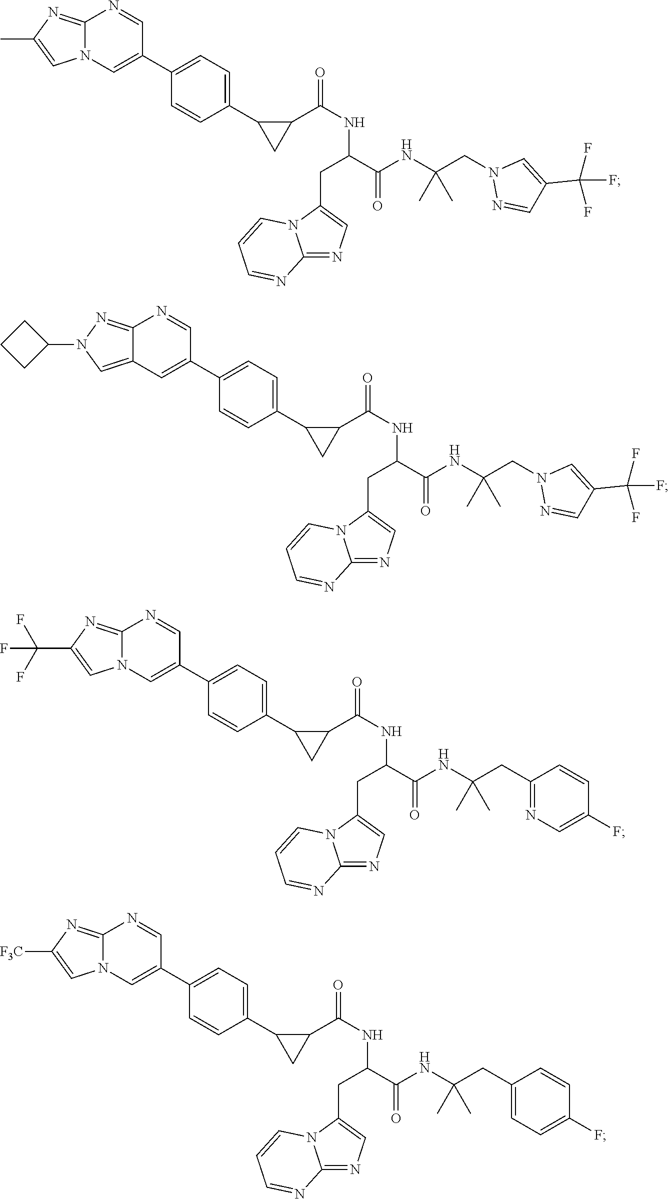 [SMILES: Cc1cn2cc(-c3ccc(C4CC4C(=O)NC(Cc4cnc5ncccn45)C(=O)NC(C)(C)Cn4cc(C(F)(F)F)cn4)cc3)cnc2n1 ; CC(C)(Cn1cc(C(F)(F)F)cn1)NC(=O)C(Cc1cnc2ncccn12)NC(=O)C1CC1c1ccc(-c2cnc3nn(C4CCC4)cc3c2)cc1 ; CC(C)(Cc1ccc(F)cn1)NC(=O)C(Cc1cnc2ncccn12)NC(=O)C1CC1c1ccc(-c2cnc3nc(C(F)(F)F)cn3c2)cc1 ; CC(C)(Cc1ccc(F)cc1)NC(=O)C(Cc1cnc2ncccn12)NC(=O)C1CC1c1ccc(-c2cnc3nc(C(F)(F)F)cn3c2)cc1]

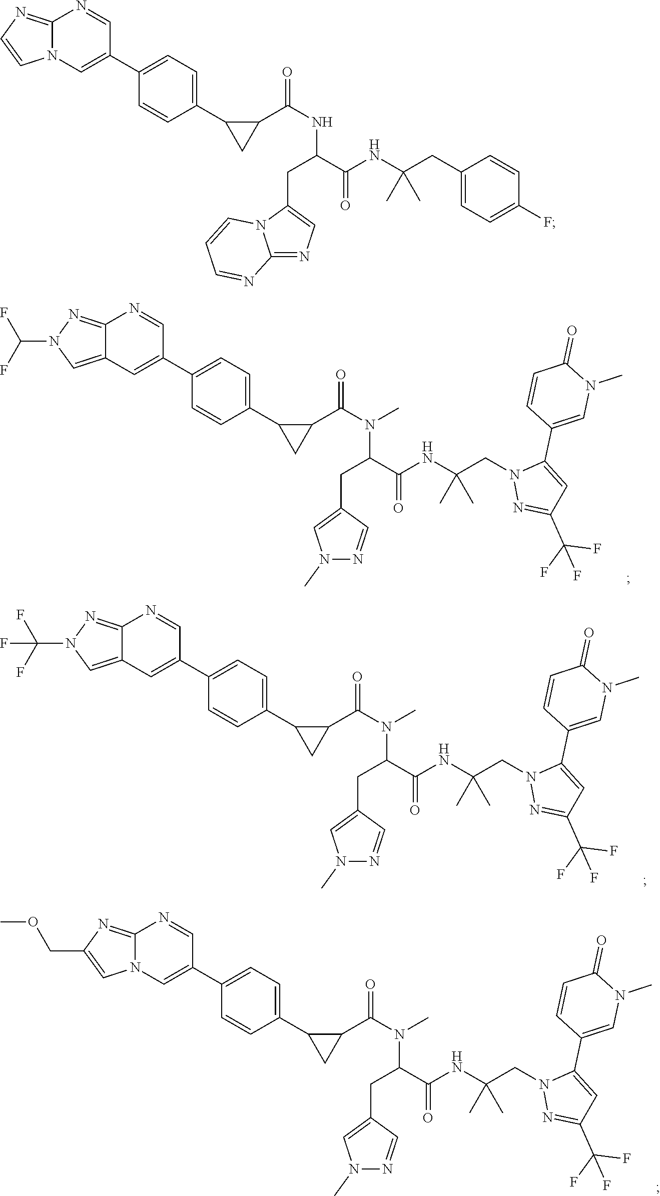

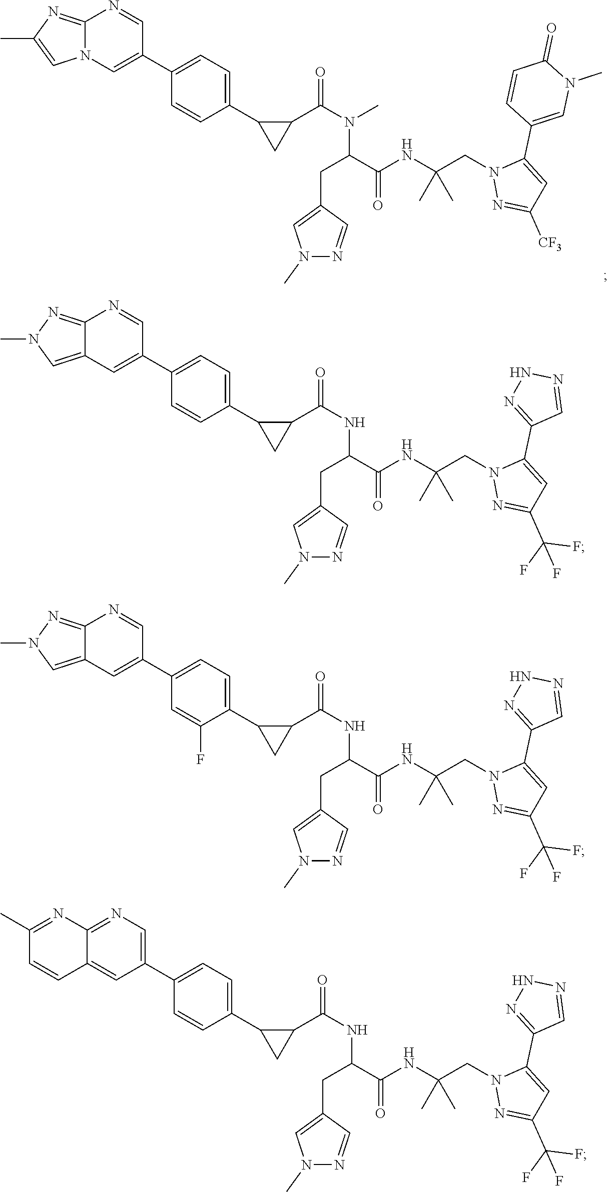

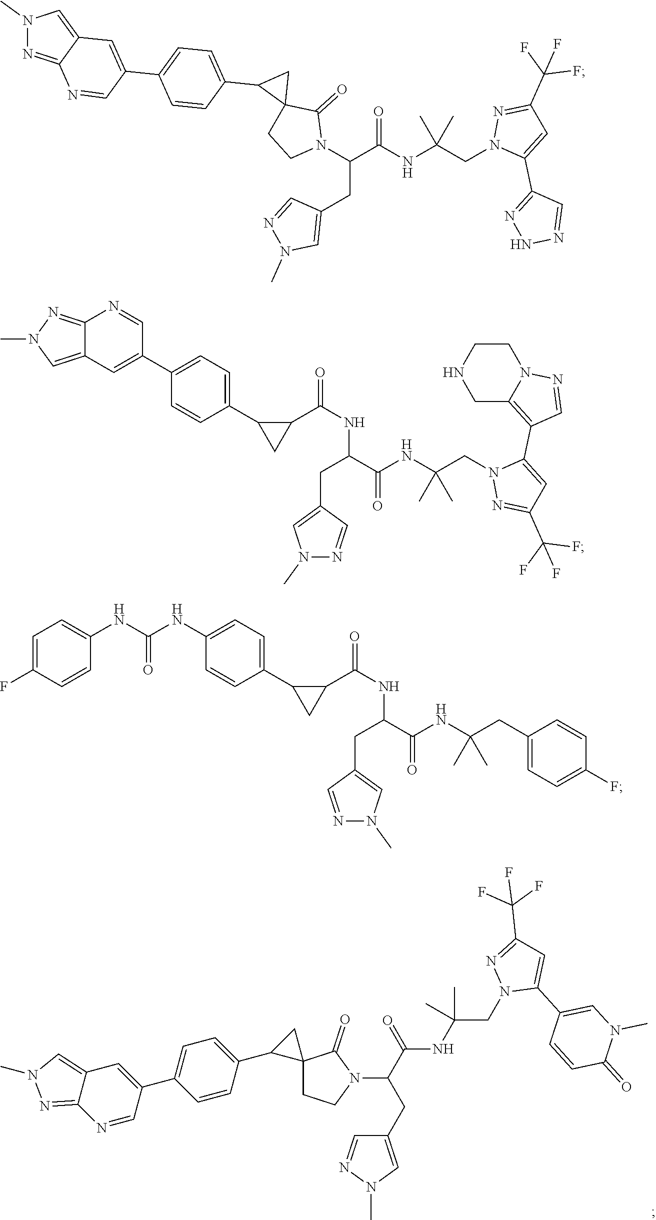

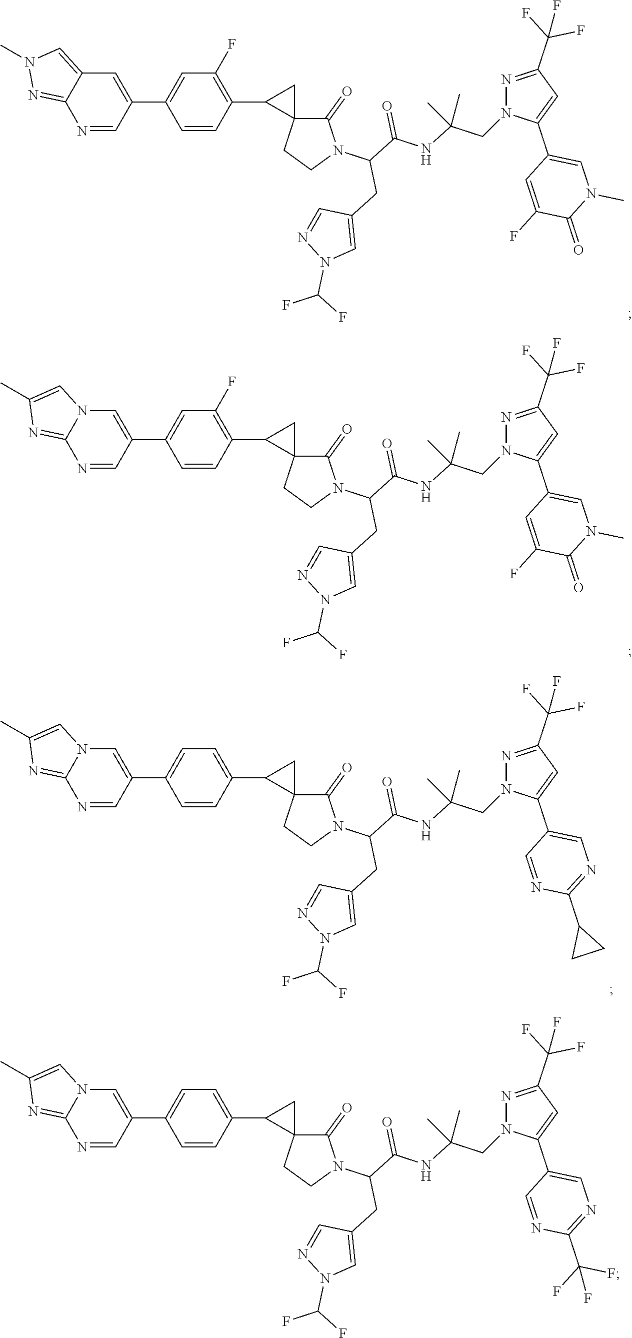

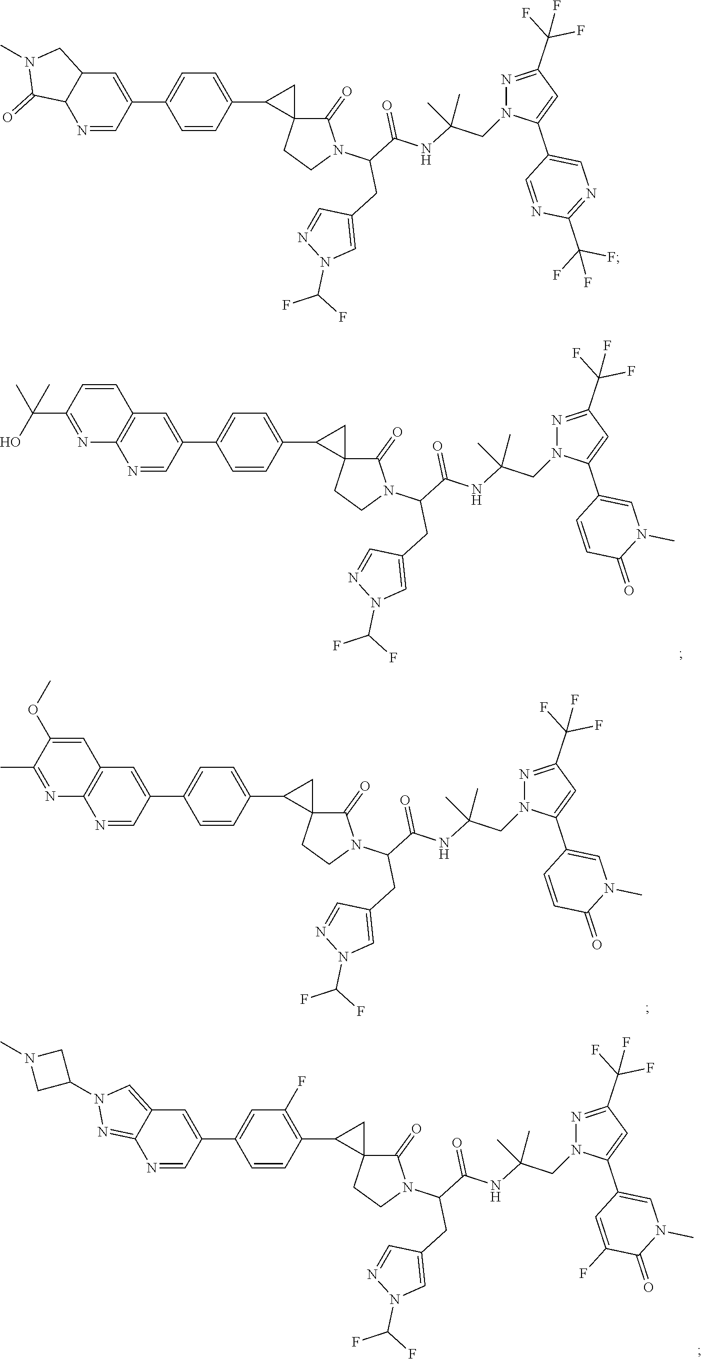

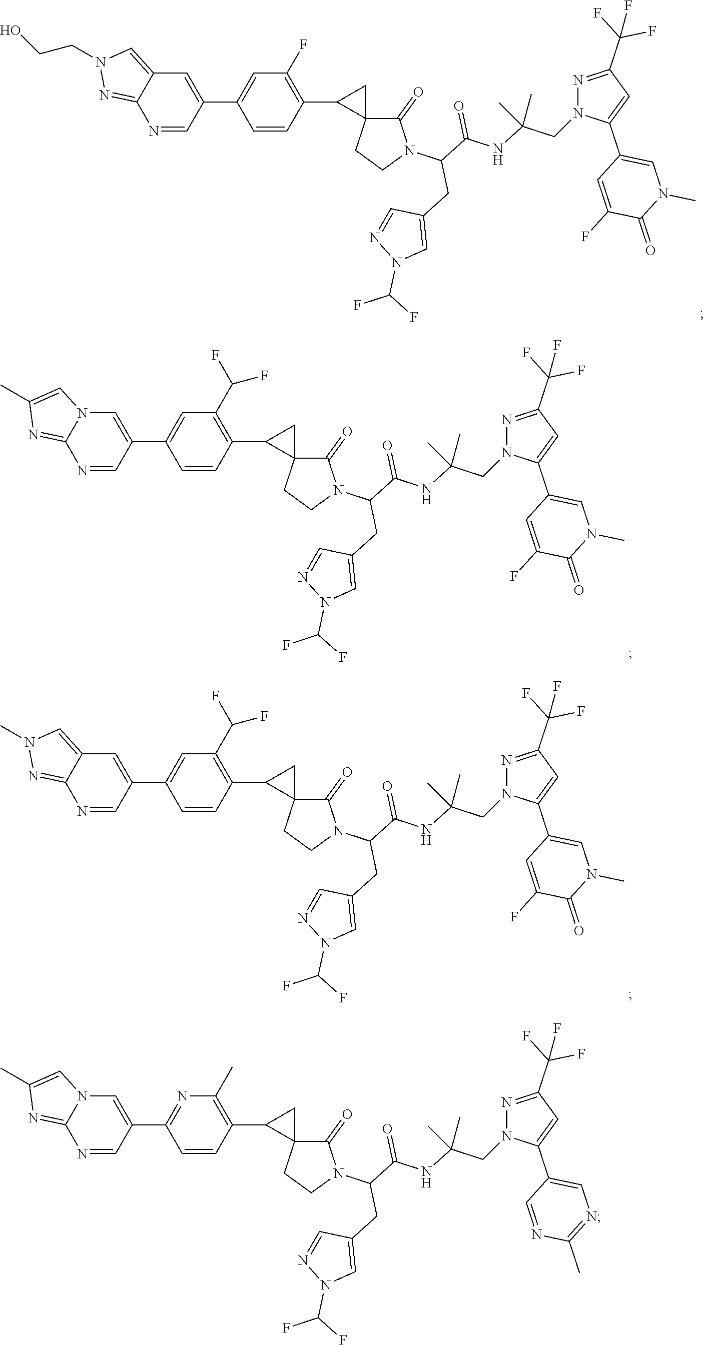

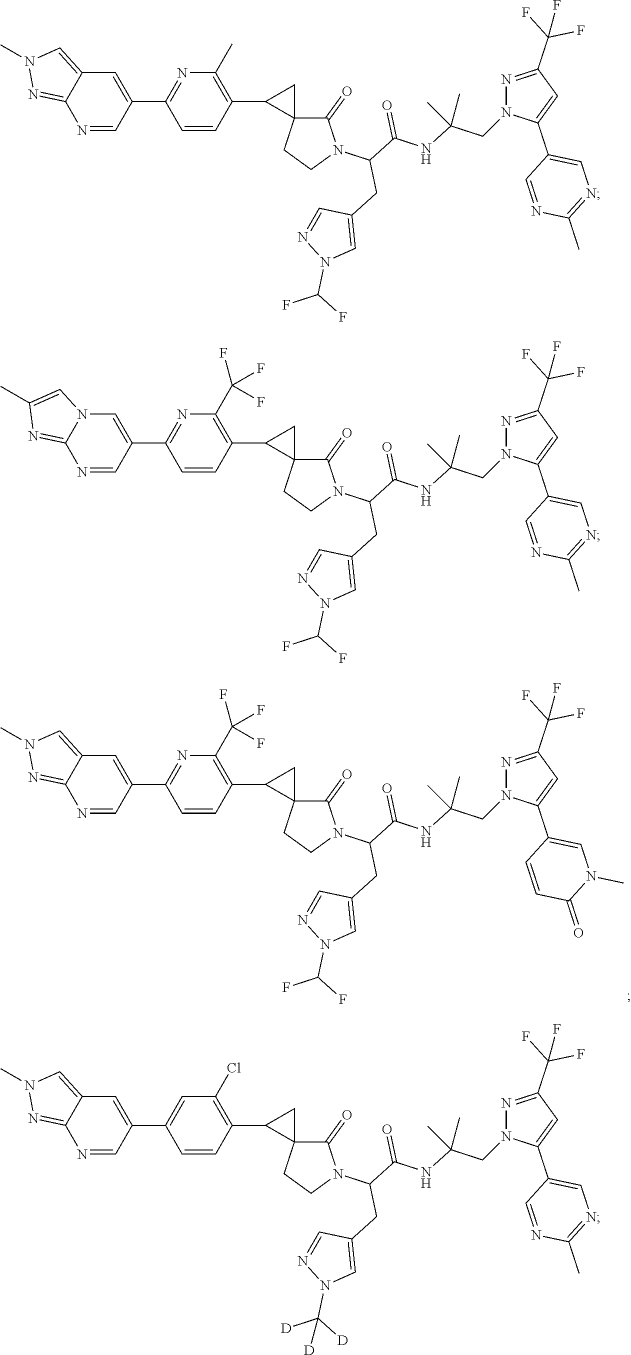

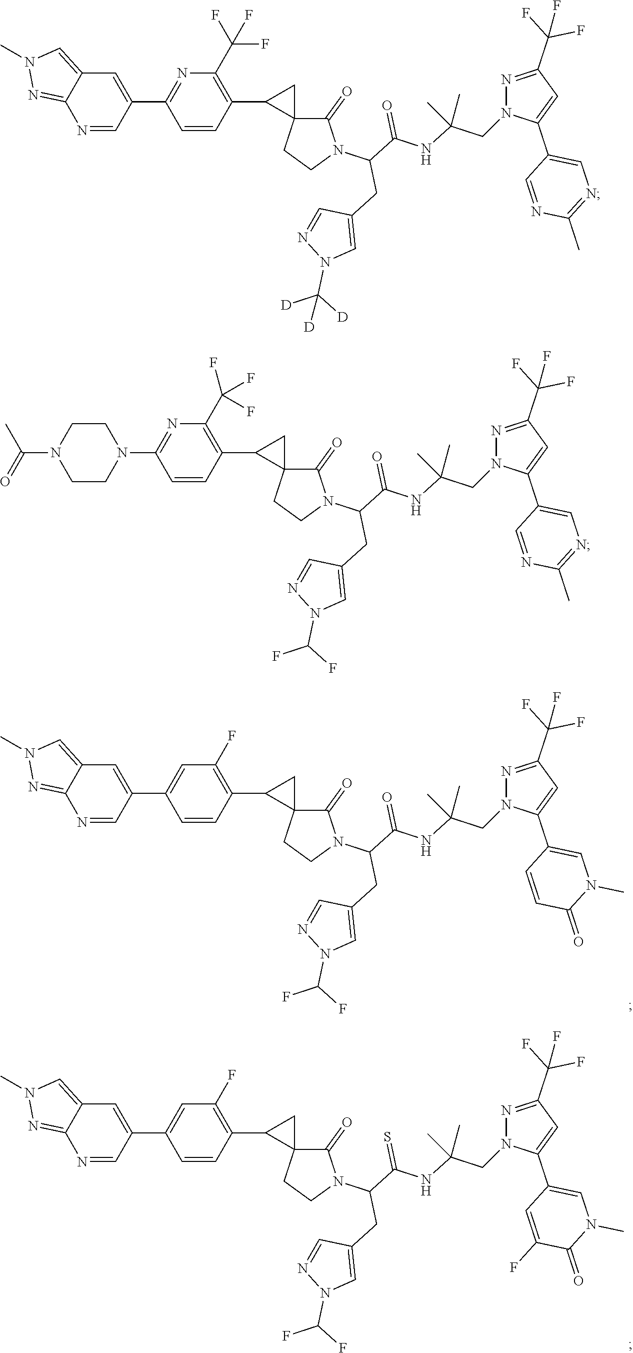

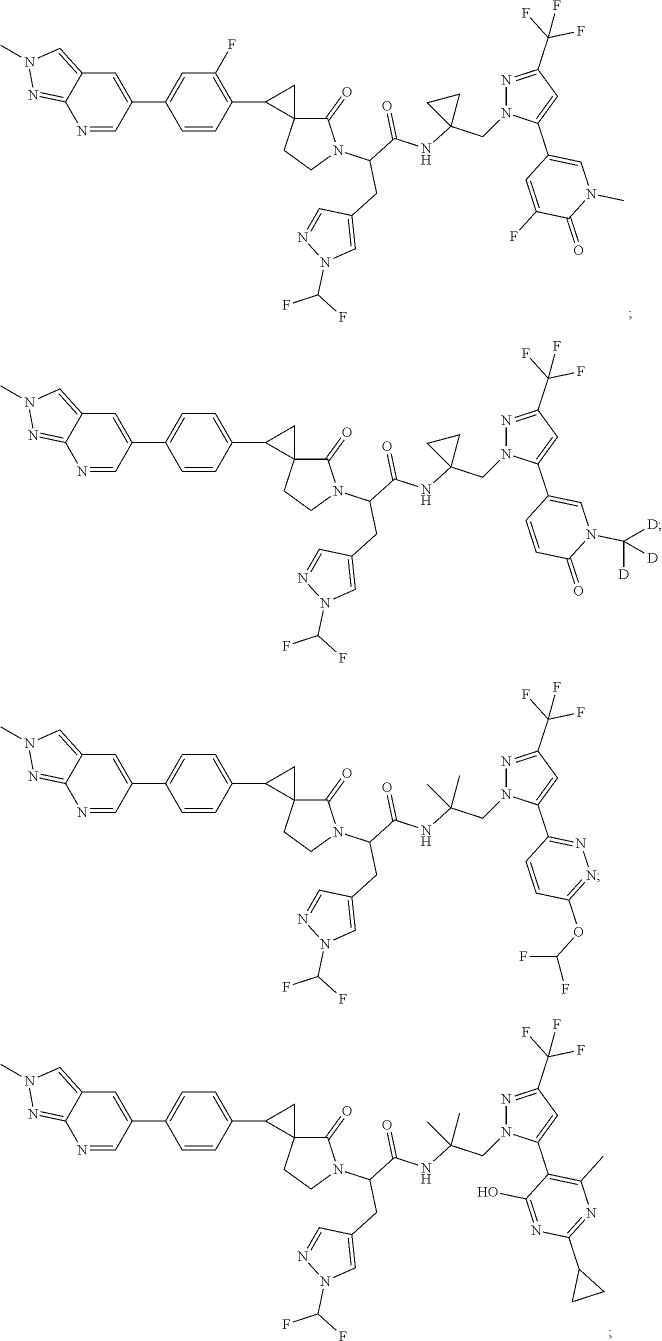

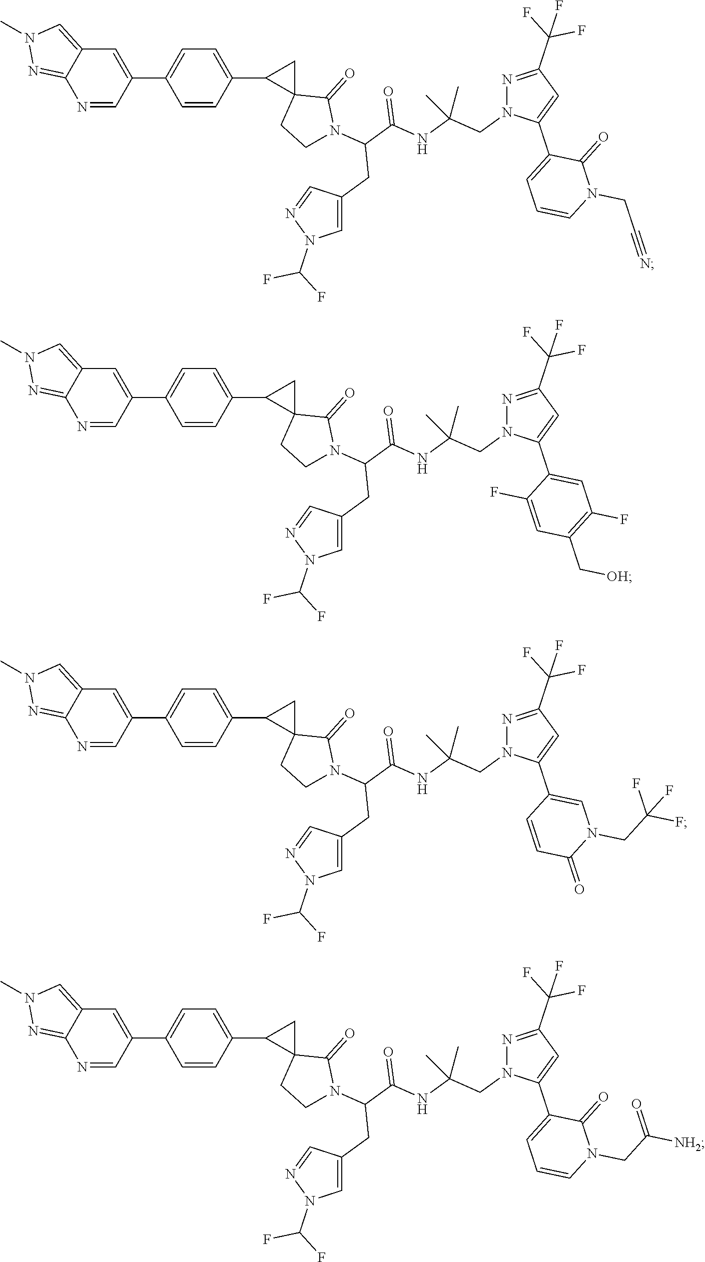 [SMILES: Cn1cc2cc(-c3ccc(C4CC45CCN(C(Cc4cnn(C(F)F)c4)C(=O)NC(C)(C)Cn4nc(C(F)(F)F)cc4-c4cccn(CC#N)c4=O)C5=O)cc3)cnc2n1 ; Cn1cc2cc(-c3ccc(C4CC45CCN(C(Cc4cnn(C(F)F)c4)C(=O)NC(C)(C)Cn4nc(C(F)(F)F)cc4-c4cc(F)c(CO)cc4F)C5=O)cc3)cnc2n1 ; Cn1cc2cc(-c3ccc(C4CC45CCN(C(Cc4cnn(C(F)F)c4)C(=O)NC(C)(C)Cn4nc(C(F)(F)F)cc4-c4ccc(=O)n(CC(F)(F)F)c4)C5=O)cc3)cnc2n1 ; Cn1cc2cc(-c3ccc(C4CC45CCN(C(Cc4cnn(C(F)F)c4)C(=O)NC(C)(C)Cn4nc(C(F)(F)F)cc4-c4cccn(CC(N)=O)c4=O)C5=O)cc3)cnc2n1]

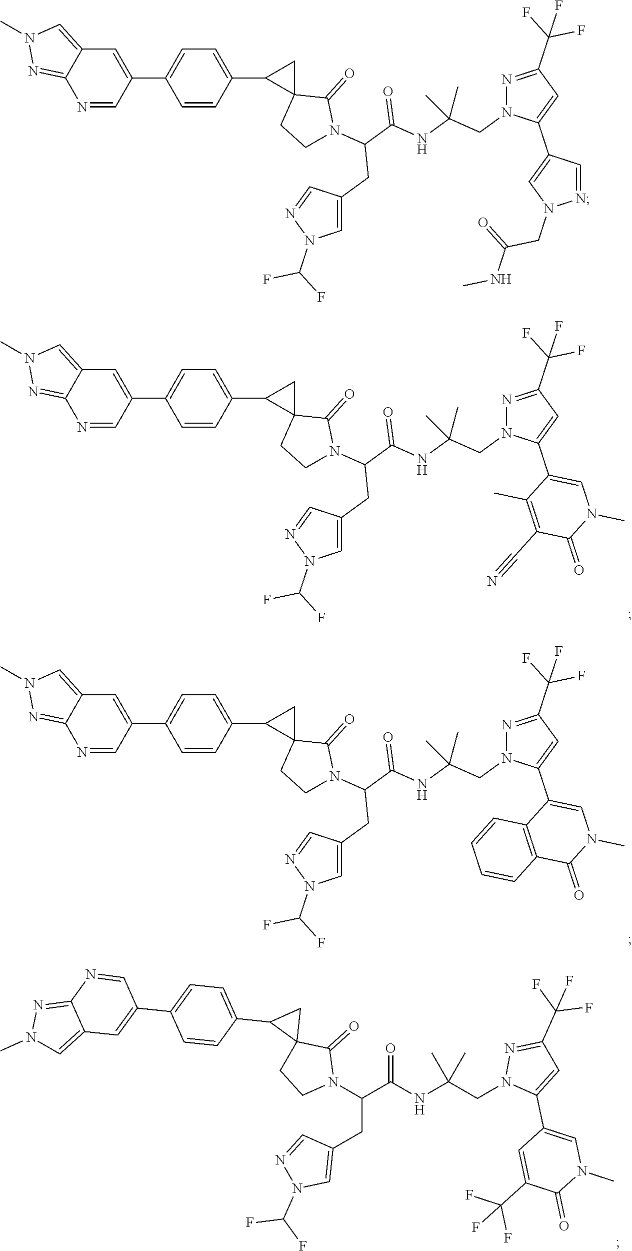

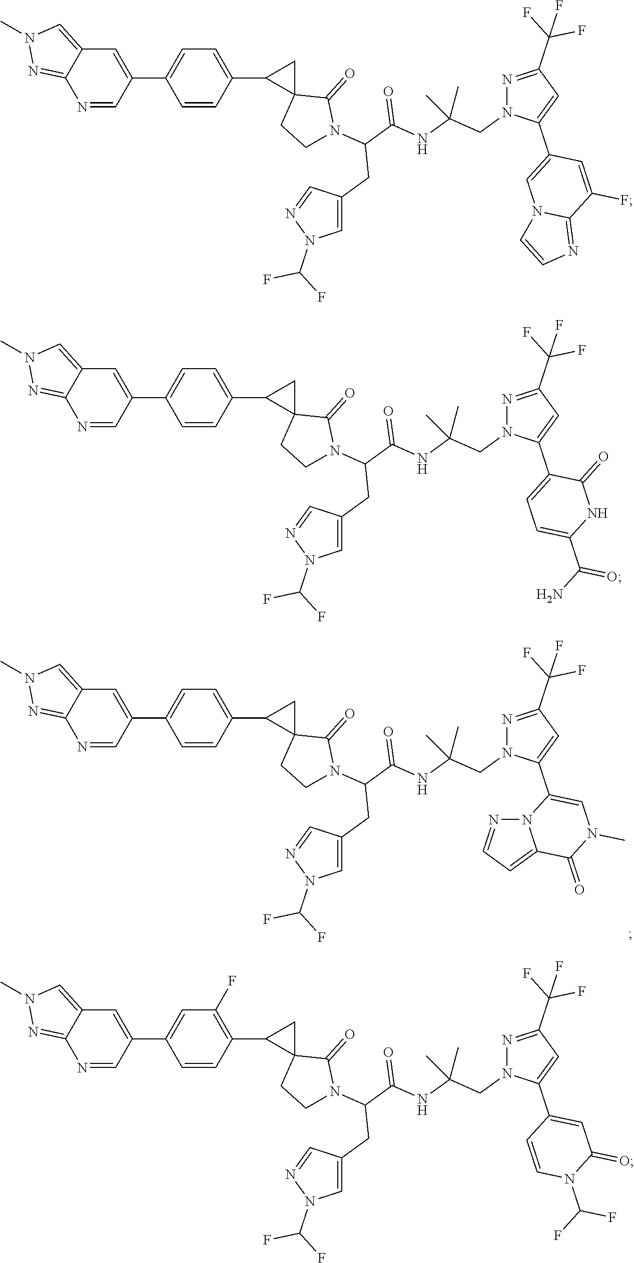

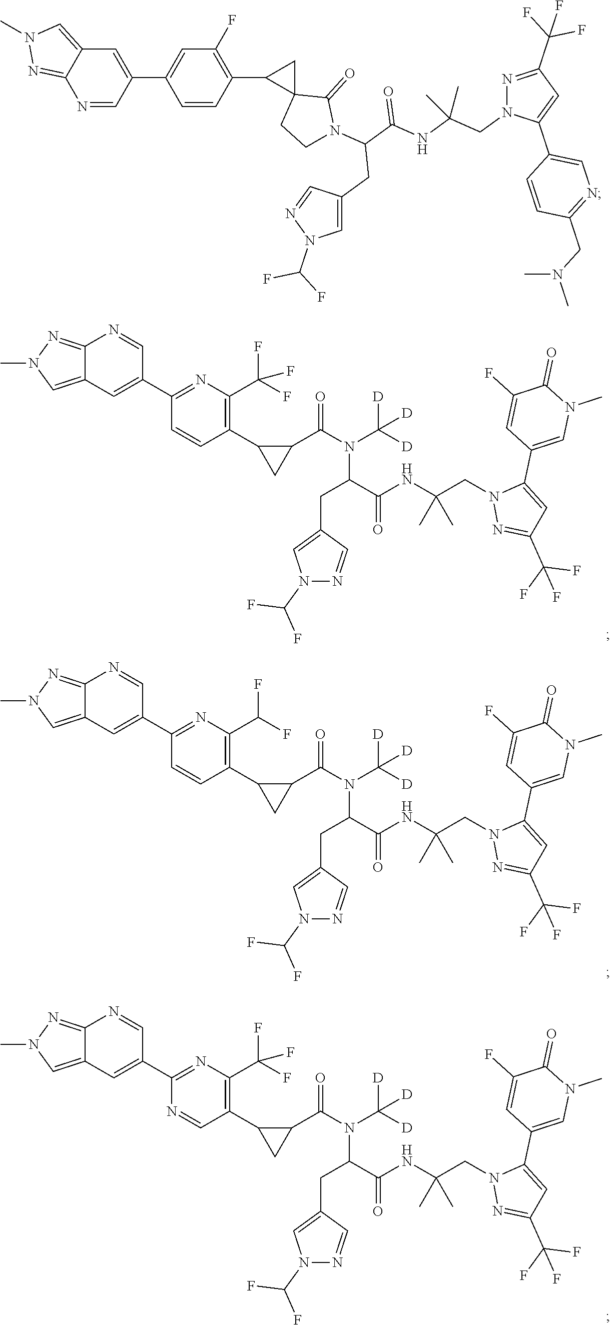

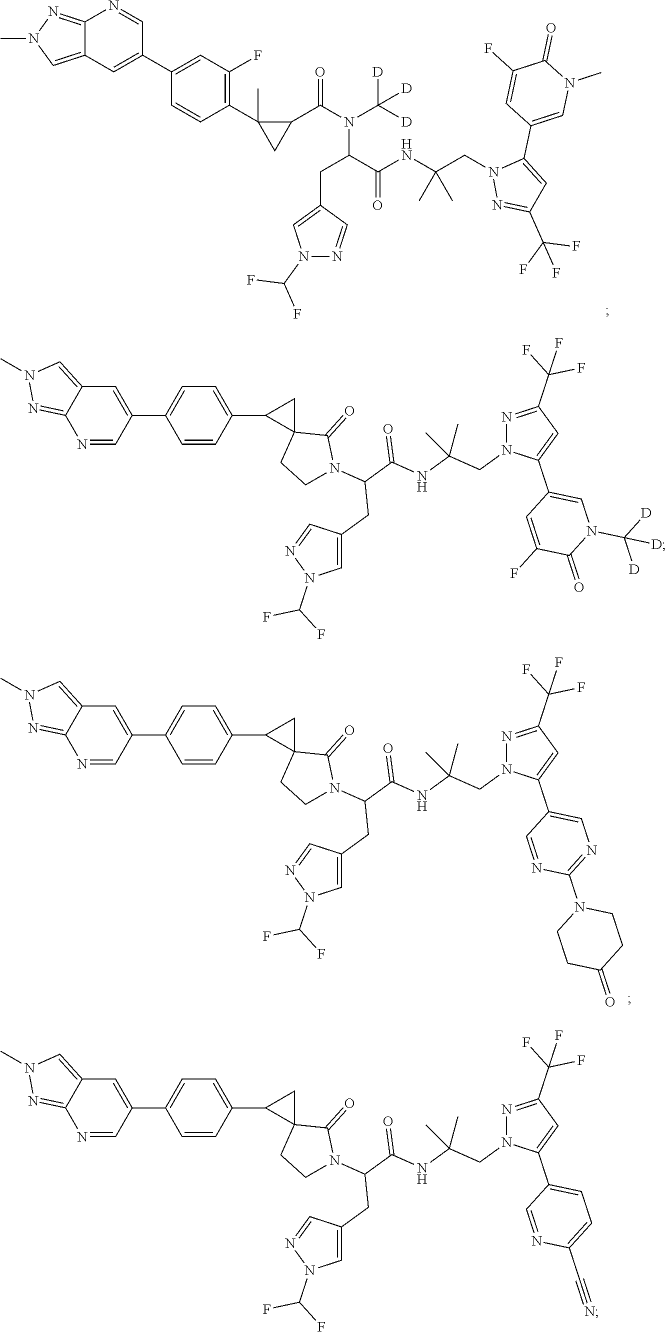

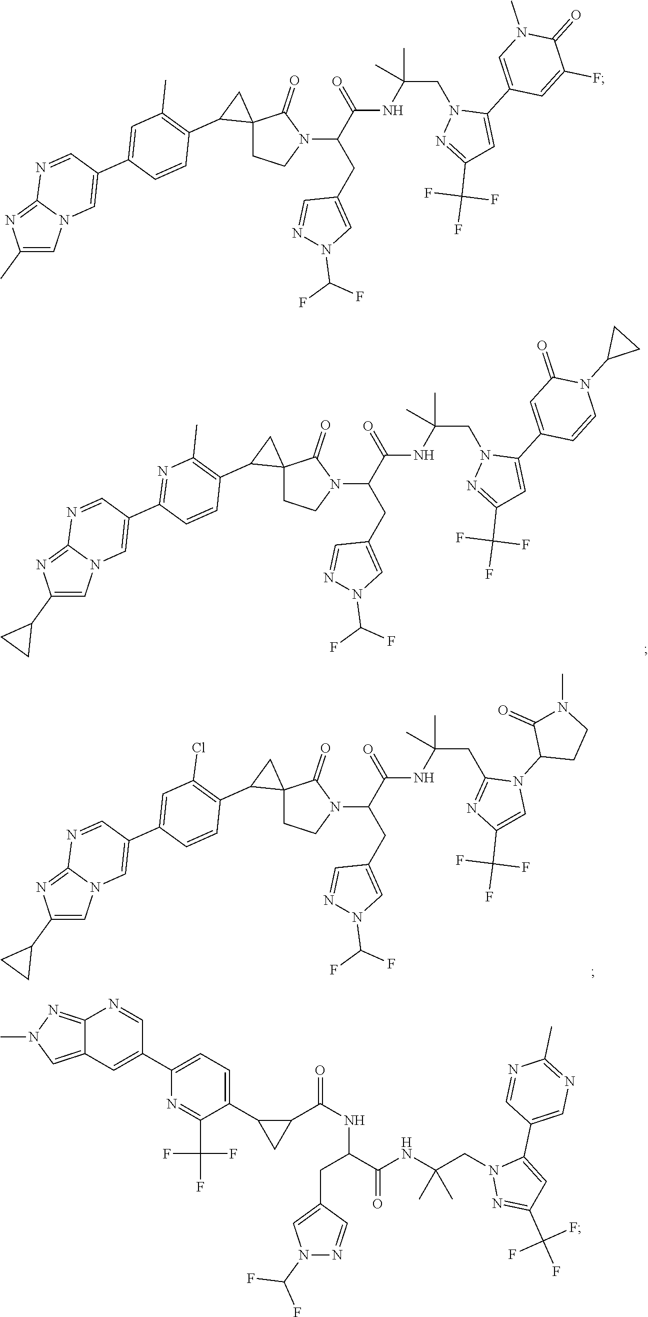

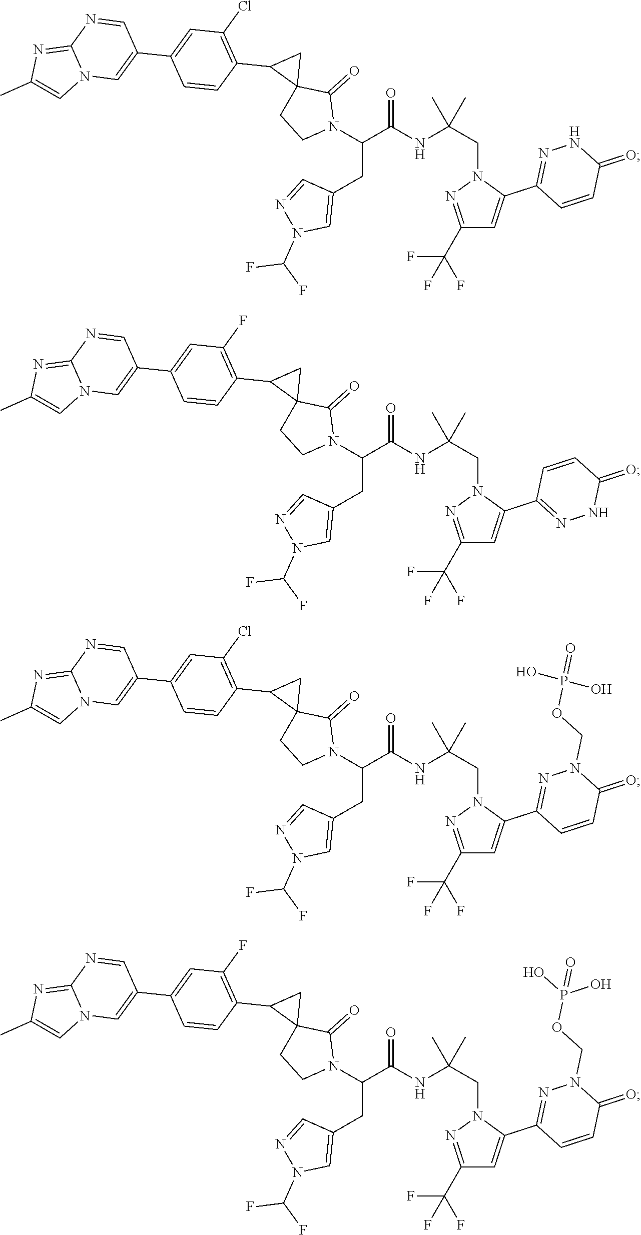 [SMILES: Cc1cn2cc(-c3ccc(C4CC45CCN(C(Cc4cnn(C(F)F)c4)C(=O)NC(C)(C)Cn4nc(C(F)(F)F)cc4-c4ccc(=O)[nH]n4)C5=O)c(Cl)c3)cnc2n1 ; Cc1cn2cc(-c3ccc(C4CC45CCN(C(Cc4cnn(C(F)F)c4)C(=O)NC(C)(C)Cn4nc(C(F)(F)F)cc4-c4ccc(=O)[nH]n4)C5=O)c(F)c3)cnc2n1 ; Cc1cn2cc(-c3ccc(C4CC45CCN(C(Cc4cnn(C(F)F)c4)C(=O)NC(C)(C)Cn4nc(C(F)(F)F)cc4-c4ccc(=O)n(COP(=O)(O)O)n4)C5=O)c(Cl)c3)cnc2n1 ; Cc1cn2cc(-c3ccc(C4CC45CCN(C(Cc4cnn(C(F)F)c4)C(=O)NC(C)(C)Cn4nc(C(F)(F)F)cc4-c4ccc(=O)n(COP(=O)(O)O)n4)C5=O)c(F)c3)cnc2n1]

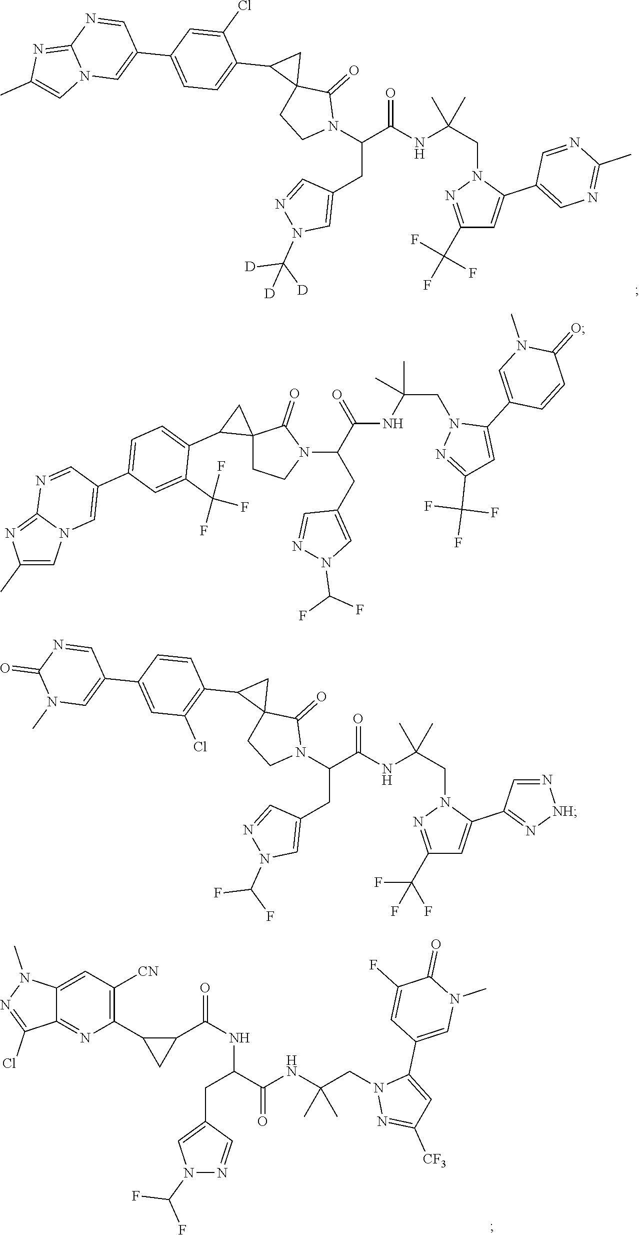

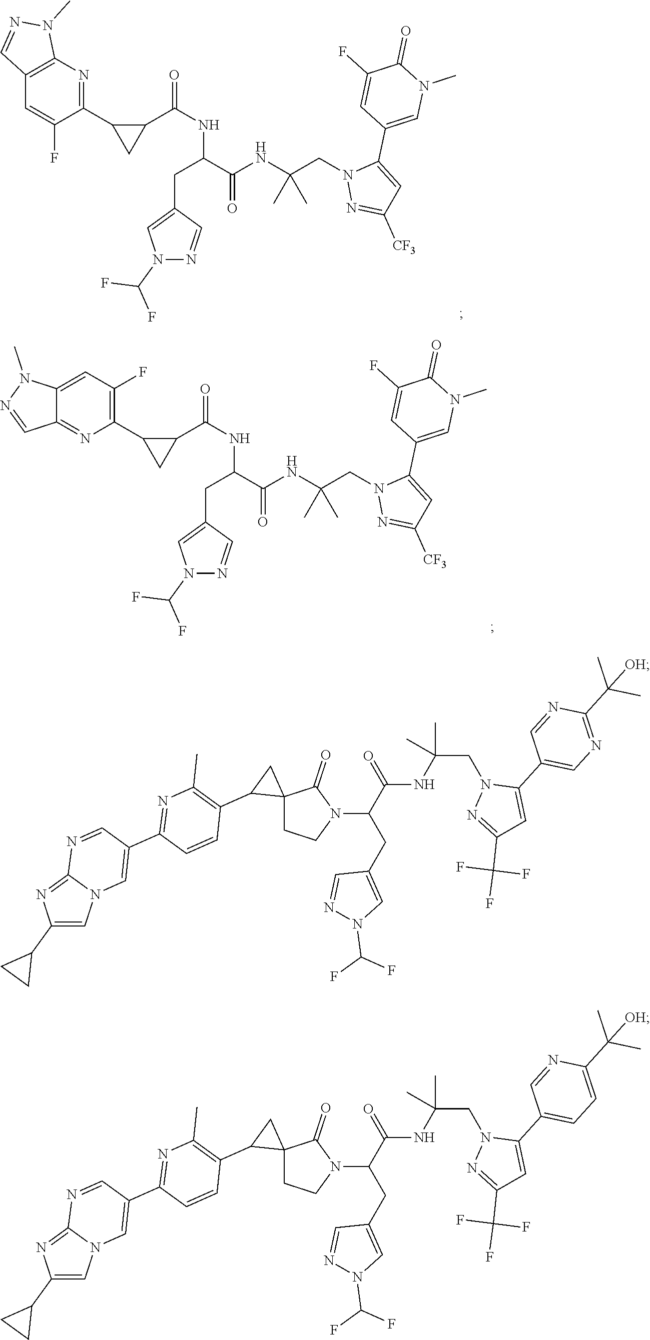 [SMILES: Cn1cc(-c2cc(C(F)(F)F)nn2CC(C)(C)NC(=O)C(Cc2cnn(C(F)F)c2)NC(=O)C2CC2c2nc3c(cnn3C)cc2F)cc(F)c1=O ; Cn1cc(-c2cc(C(F)(F)F)nn2CC(C)(C)NC(=O)C(Cc2cnn(C(F)F)c2)NC(=O)C2CC2c2nc3cnn(C)c3cc2F)cc(F)c1=O ; Cc1nc(-c2cnc3nc(C4CC4)cn3c2)ccc1C1CC12CCN(C(Cc1cnn(C(F)F)c1)C(=O)NC(C)(C)Cn1nc(C(F)(F)F)cc1-c1cnc(C(C)(C)O)nc1)C2=O ; Cc1nc(-c2cnc3nc(C4CC4)cn3c2)ccc1C1CC12CCN(C(Cc1cnn(C(F)F)c1)C(=O)NC(C)(C)Cn1nc(C(F)(F)F)cc1-c1ccc(C(C)(C)O)nc1)C2=O]

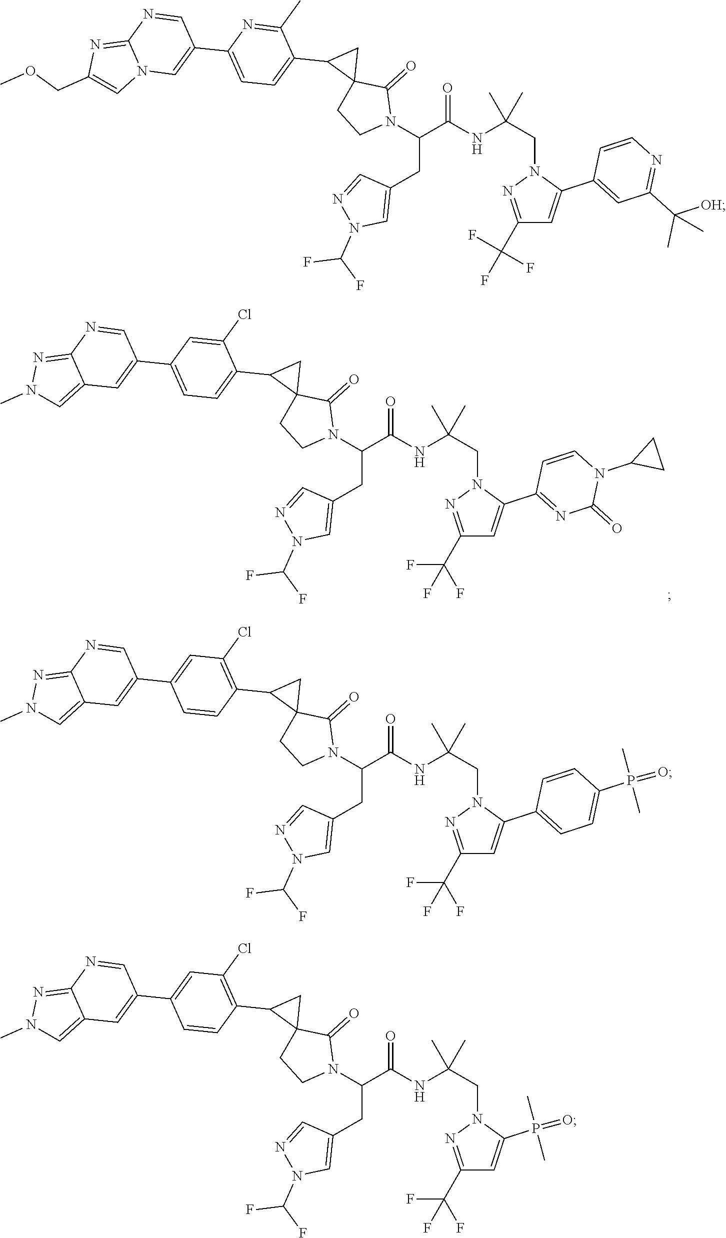 [SMILES: COCc1cn2cc(-c3ccc(C4CC45CCN(C(Cc4cnn(C(F)F)c4)C(=O)NC(C)(C)Cn4nc(C(F)(F)F)cc4-c4ccnc(C(C)(C)O)c4)C5=O)c(C)n3)cnc2n1 ; Cn1cc2cc(-c3ccc(C4CC45CCN(C(Cc4cnn(C(F)F)c4)C(=O)NC(C)(C)Cn4nc(C(F)(F)F)cc4-c4ccn(C6CC6)c(=O)n4)C5=O)c(Cl)c3)cnc2n1 ; Cn1cc2cc(-c3ccc(C4CC45CCN(C(Cc4cnn(C(F)F)c4)C(=O)NC(C)(C)Cn4nc(C(F)(F)F)cc4-c4ccc(P(C)(C)=O)cc4)C5=O)c(Cl)c3)cnc2n1 ; Cn1cc2cc(-c3ccc(C4CC45CCN(C(Cc4cnn(C(F)F)c4)C(=O)NC(C)(C)Cn4nc(C(F)(F)F)cc4P(C)(C)=O)C5=O)c(Cl)c3)cnc2n1]

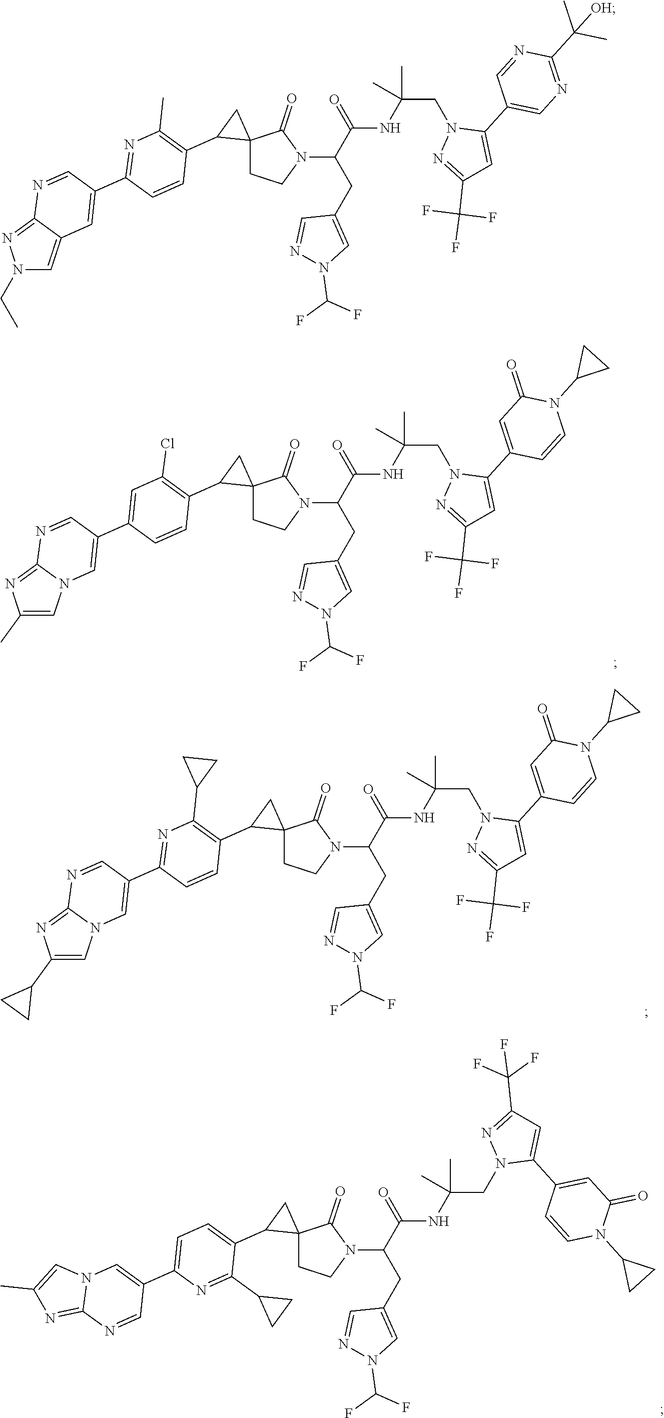

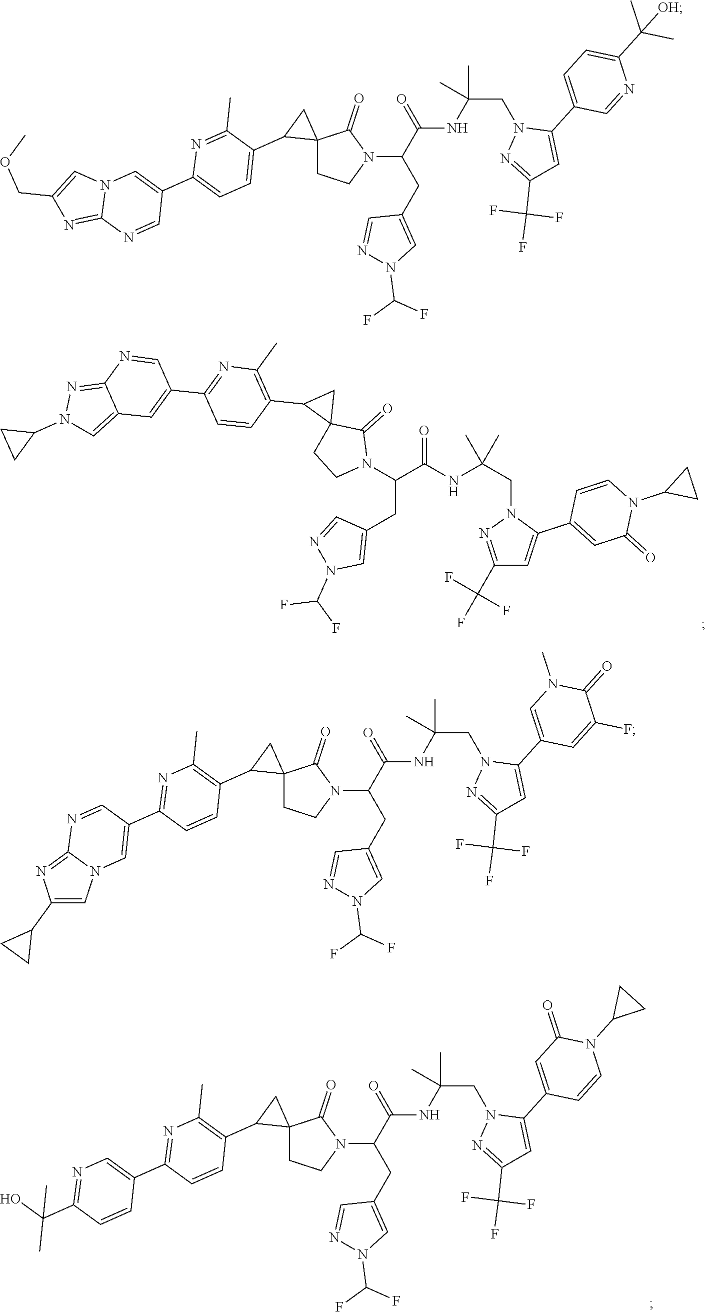 [SMILES: COCc1cn2cc(-c3ccc(C4CC45CCN(C(Cc4cnn(C(F)F)c4)C(=O)NC(C)(C)Cn4nc(C(F)(F)F)cc4-c4ccc(C(C)(C)O)nc4)C5=O)c(C)n3)cnc2n1 ; Cc1nc(-c2cnc3nn(C4CC4)cc3c2)ccc1C1CC12CCN(C(Cc1cnn(C(F)F)c1)C(=O)NC(C)(C)Cn1nc(C(F)(F)F)cc1-c1ccn(C3CC3)c(=O)c1)C2=O ; Cc1nc(-c2cnc3nc(C4CC4)cn3c2)ccc1C1CC12CCN(C(Cc1cnn(C(F)F)c1)C(=O)NC(C)(C)Cn1nc(C(F)(F)F)cc1-c1cc(F)c(=O)n(C)c1)C2=O ; Cc1nc(-c2ccc(C(C)(C)O)nc2)ccc1C1CC12CCN(C(Cc1cnn(C(F)F)c1)C(=O)NC(C)(C)Cn1nc(C(F)(F)F)cc1-c1ccn(C3CC3)c(=O)c1)C2=O]

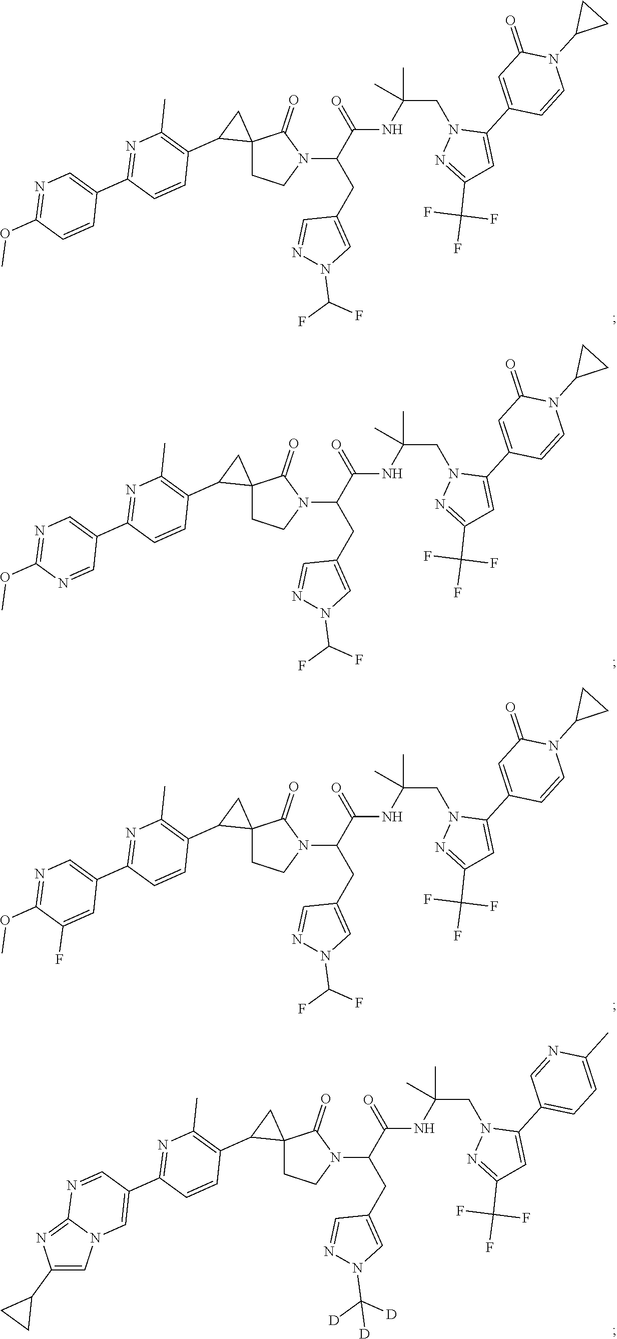 [SMILES: COc1ccc(-c2ccc(C3CC34CCN(C(Cc3cnn(C(F)F)c3)C(=O)NC(C)(C)Cn3nc(C(F)(F)F)cc3-c3ccn(C5CC5)c(=O)c3)C4=O)c(C)n2)cn1 ; COc1ncc(-c2ccc(C3CC34CCN(C(Cc3cnn(C(F)F)c3)C(=O)NC(C)(C)Cn3nc(C(F)(F)F)cc3-c3ccn(C5CC5)c(=O)c3)C4=O)c(C)n2)cn1 ; COc1ncc(-c2ccc(C3CC34CCN(C(Cc3cnn(C(F)F)c3)C(=O)NC(C)(C)Cn3nc(C(F)(F)F)cc3-c3ccn(C5CC5)c(=O)c3)C4=O)c(C)n2)cc1F ; [2H]C([2H])([2H])n1cc(CC(C(=O)NC(C)(C)Cn2nc(C(F)(F)F)cc2-c2ccc(C)nc2)N2CCC3(CC3c3ccc(-c4cnc5nc(C6CC6)cn5c4)nc3C)C2=O)cn1]

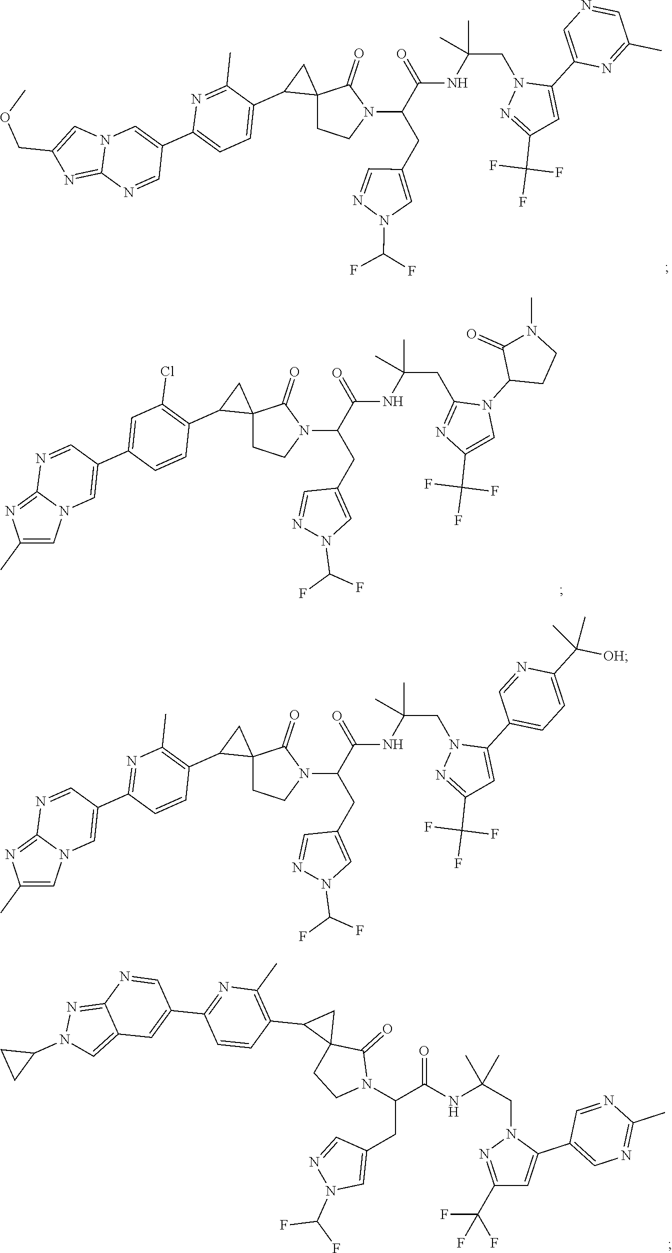

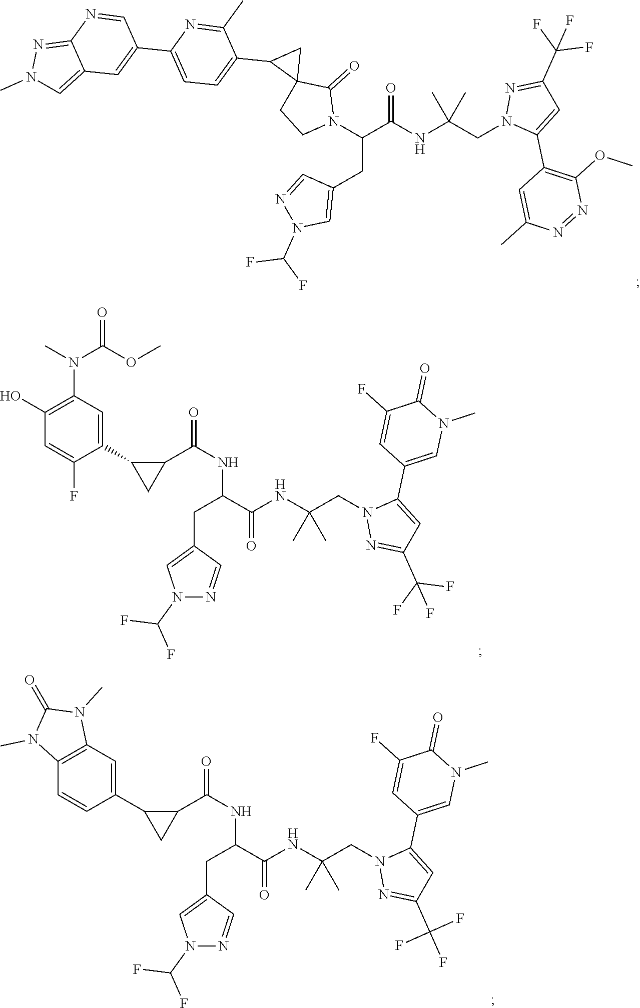

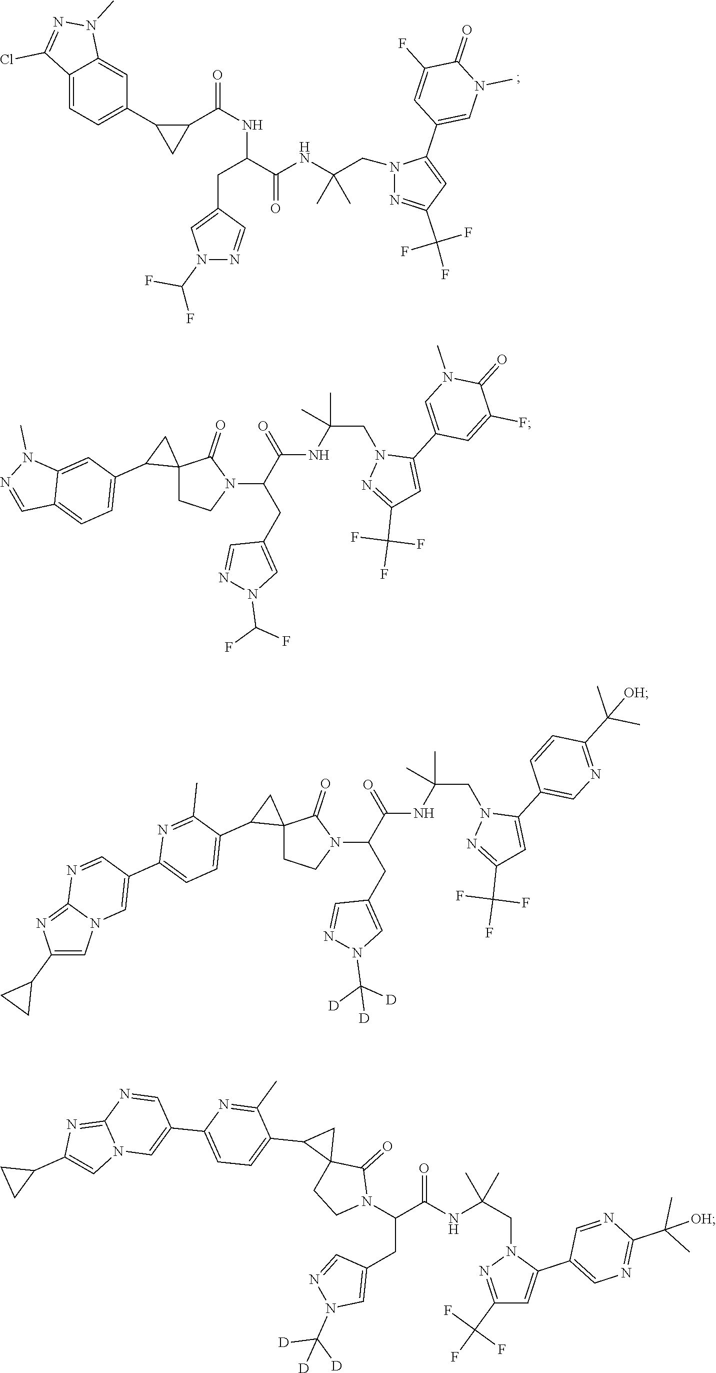 [SMILES: Cn1cc(-c2cc(C(F)(F)F)nn2CC(C)(C)NC(=O)C(Cc2cnn(C(F)F)c2)NC(=O)C2CC2c2ccc3c(Cl)nn(C)c3c2)cc(F)c1=O ; Cn1cc(-c2cc(C(F)(F)F)nn2CC(C)(C)NC(=O)C(Cc2cnn(C(F)F)c2)N2CCC3(CC3c3ccc4cnn(C)c4c3)C2=O)cc(F)c1=O ; [2H]C([2H])([2H])n1cc(CC(C(=O)NC(C)(C)Cn2nc(C(F)(F)F)cc2-c2ccc(C(C)(C)O)nc2)N2CCC3(CC3c3ccc(-c4cnc5nc(C6CC6)cn5c4)nc3C)C2=O)cn1 ; [2H]C([2H])([2H])n1cc(CC(C(=O)NC(C)(C)Cn2nc(C(F)(F)F)cc2-c2cnc(C(C)(C)O)nc2)N2CCC3(CC3c3ccc(-c4cnc5nc(C6CC6)cn5c4)nc3C)C2=O)cn1]

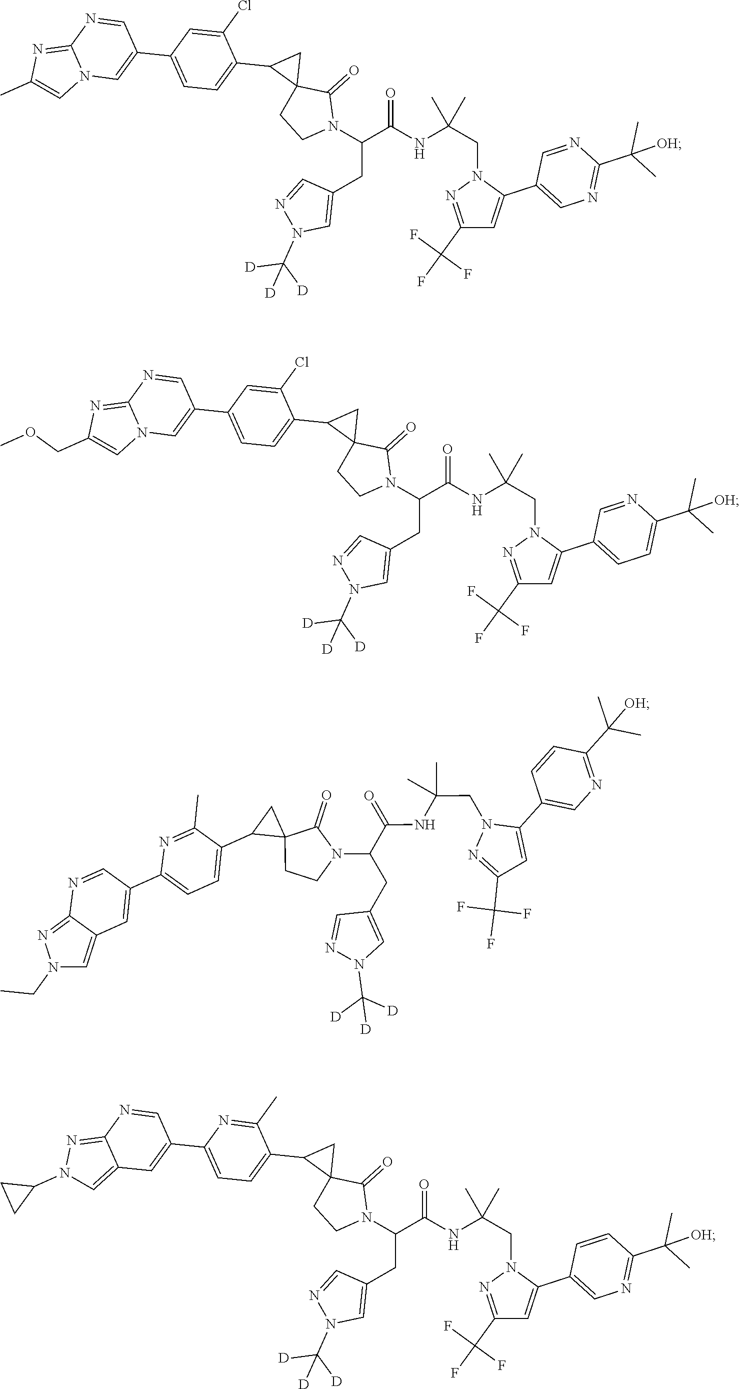

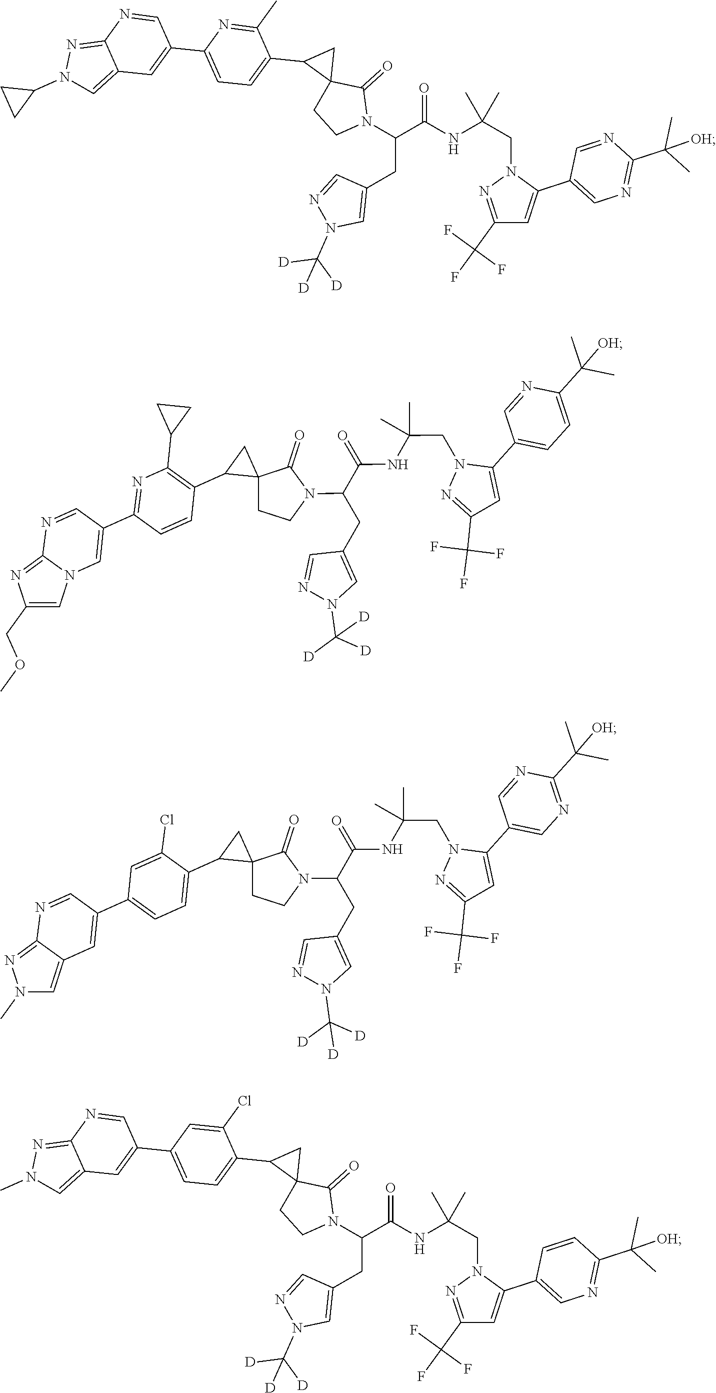

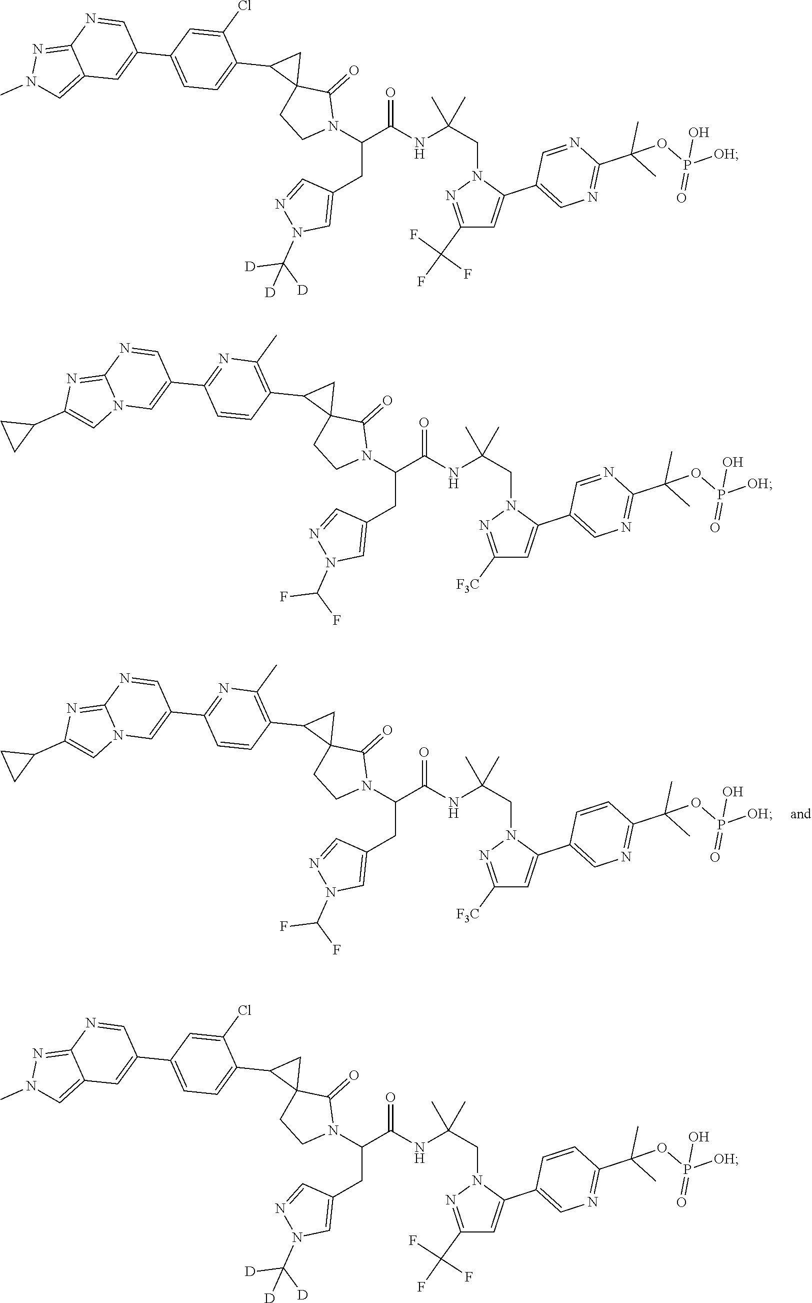

or a pharmaceutically acceptable salt thereof.

### NUMBERED EMBODIMENTS

1. A compound of formula:

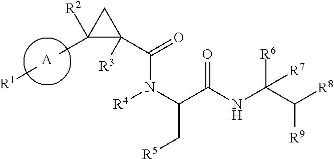

wherein
R¹ is:
   i) a 9- or 10-membered N-containing bicyclic heterocycle optionally substituted with 1 or 2 substituents independently selected from:
      C₃-C₄cycloalkyl optionally substituted with 1 or 2 halo or CH₃,
      C₁-C₄haloalkyl,
      oxo, and
      C₁-C₄alkyl optionally substituted with OCH₃ or C₃-C₄cycloalkyl, wherein the C₃-C₄cycloalkyl is optionally substituted with 1 or 2 halo,
   ii) NHCOR¹², or
   iii) OH;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
R² is H, CH₃, halo, CF₃ or CHF₂;
R³ is H and R⁴ is H or CH₃; or R³ and R⁴ together form —CH₂—CH₂—;
R⁵ is:
   i) a 8- or 9-membered N-containing bicyclic heterocycle optionally substituted with halo, or
   ii) a 5- or 6-membered N-containing heteroaryl optionally substituted with CH₃, CHF₂ or OH;
R⁶ and R⁷ are each CH₃ or together form a cyclopropyl;
R⁸ is H, CH₃, CO₂H or C(O)OCH₃ and R⁹ is:
   phenyl optionally substituted with 1 or 2 substituents selected from: halo, CF₃ and pyrazine optionally substituted with CF₂H or OCH₃, or
   a 5- or 6-membered N-containing heteroaryl optionally substituted with R¹⁰ or with R¹⁰ and R¹¹;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
   i) a 5- or 6-membered heterocycle optionally substituted with 1 to 3 substituents independently selected from:
      halo,
      OH,
      OCH₃,
      SO₂NH₂,
      a 4- to 6-membered heterocycle,
      oxo or
      C₁-C₄alkyl, wherein the C₁-C₄alkyl is optionally substituted with OH, OCH₃, a 4- to 6-membered heterocycle or C₃-C₄cycloalkyl optionally substituted with halo,
   ii) a 9-membered N-containing bicyclic heterocycle optionally substituted with CN, oxo, C(O)O(CH₃)₃ or OH,
   iii) C₁-C₄alkoxy,
   iv) C₃-C₅cycloalkyl,
   v) halo, or
   vi) C(O)OCH₃;
or a pharmaceutically acceptable salt thereof.

2. The compound according to embodiment 1, wherein R⁶ and R⁷ are each CH₃, or a pharmaceutically acceptable salt thereof.

3. The compound according to embodiment 1 or embodiment 2, wherein R³ is H and R⁴ is H or CH₃, or a pharmaceutically acceptable salt thereof.

4. The compound according to embodiment 1 or embodiment 2, wherein R³ and R⁴ together form —CH₂—CH₂—, or a pharmaceutically acceptable salt thereof.

5. The compound according to any one of embodiments 1 to 4, wherein R² is H or CH₃, or a pharmaceutically acceptable salt thereof.

6. The compound according to any one of embodiments 1 to 5, wherein R¹ is:
i) a 9- or 10-membered N-containing bicyclic heterocycle selected from:

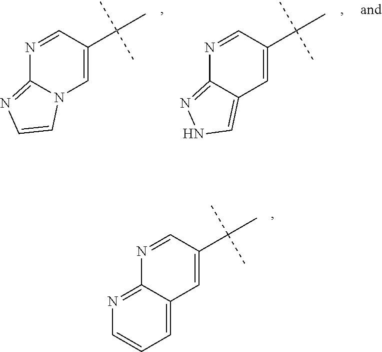

optionally substituted with 1 or 2 substituents independently selected from:
   C₃-C₄cycloalkyl optionally substituted with 1 or 2 halo or CH₃,
   C₁-C₄haloalkyl,
   C₁-C₄alkyl optionally substituted with OCH₃ or C₃-C₄cycloalkyl, wherein the C₃-C₄cycloalkyl is optionally substituted with 1 or 2 halo, or
ii) NHCOR¹², wherein R¹² is NHR¹³ or C₃-C₆cycloalkyl optionally substituted with 1 or 2 halo and R¹³ is phenyl optionally substituted with 1 or 2 halo;
or a pharmaceutically acceptable salt thereof.

7. The compound according to embodiment 6, wherein R¹ is selected from:

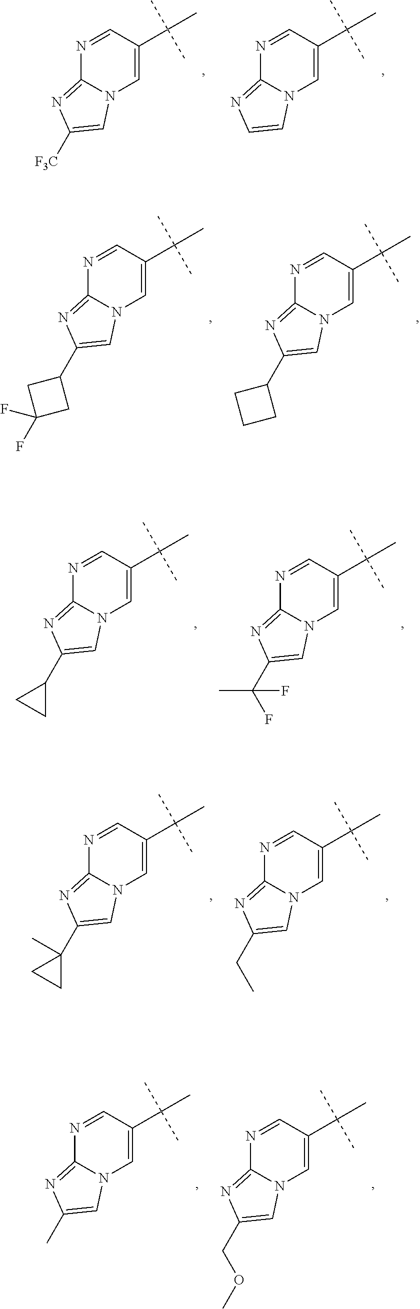

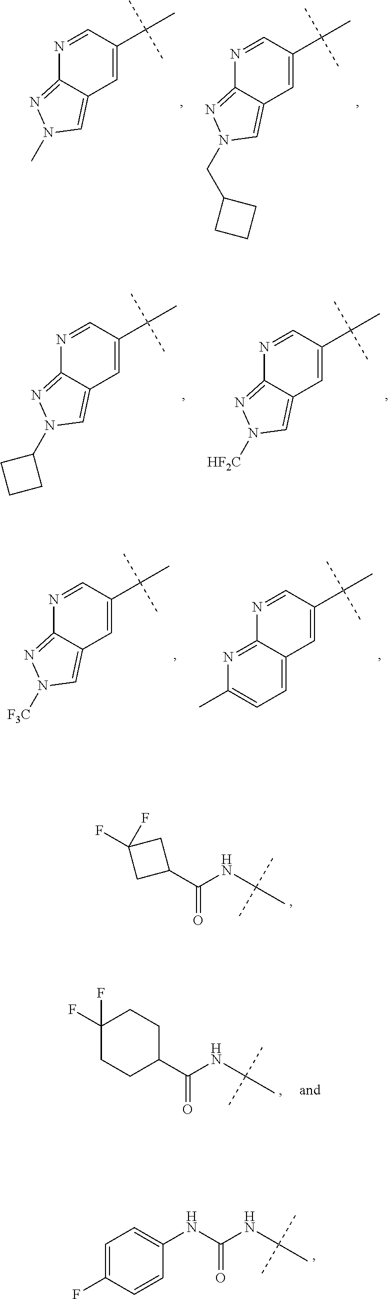

or a pharmaceutically acceptable salt thereof.

8. The compound according to any one of embodiments 1 to 7, wherein A is phenyl, pyridyl or thienyl, wherein the phenyl, pyridyl or thienyl is optionally substituted with a substituent selected from: F, CF₃ and CH₃, or a pharmaceutically acceptable salt thereof.

9. The compound according to embodiment 8, wherein A is selected from:

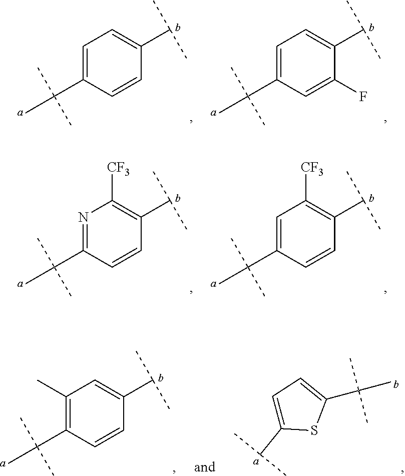

wherein a is the point of attachment to R¹ and b is the point of attachment to the cyclopropyl, or a pharmaceutically acceptable salt thereof.

10. The compound according to any one of embodiments 1 to 9, wherein R⁵ is:
a 9-membered N-containing bicyclic heterocycle, or
a 5-membered N-containing heteroaryl substituted with CH₃ or CHF₂,
or a pharmaceutically acceptable salt thereof.

11. The compound according to embodiment 10, wherein R⁵ is:

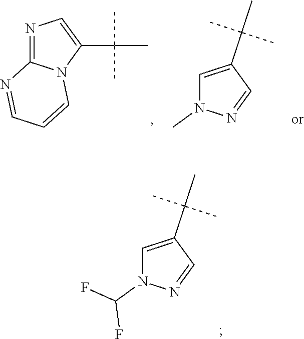

or a pharmaceutically acceptable salt thereof.

12. The compound according to any one of embodiments 1 to 11, wherein R⁸ is H, or a pharmaceutically acceptable salt thereof.

13. The compound according to any one of embodiments 1 to 12, wherein R⁹ is:phenyl substituted with F, or
a 5- or 6-membered N-containing heteroaryl optionally substituted with R¹⁰ or with R¹⁰ and R¹¹, or a pharmaceutically acceptable salt thereof.

14. The compound according to embodiment 13, wherein R⁹ is:

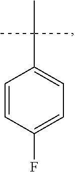

or
a 5- or 6-membered N-containing heteroaryl selected from:

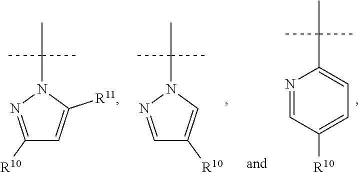

or a pharmaceutically acceptable salt thereof.

15. The compound according to embodiment 14, wherein R⁹ is:
a 5- or 6-membered N-containing heteroaryl selected from:

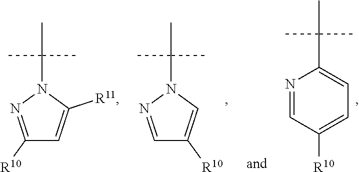

R¹⁰ is CF₃, CF₂H or F, and R¹¹ is:
   i) a 5- or 6-membered heterocycle optionally substituted with 1 or 2 substituents independently selected from: halo, a 4- to 6-membered heterocycle, oxo or C₁-C₄alkyl, wherein said C₁-C₄ alkyl is optionally substituted with OH, OCH₃, a 4- to 6-membered heterocycle and C₃-C₄cycloalkyl optionally substituted with halo,
   ii) a 9-membered N-containing bicyclic heterocycle optionally substituted with oxo or C(O)O(CH₃)₃,
   iii) C₃-C₅cycloalkyl,
   iv) halo, or
   v) C(O)OCH₃, or a pharmaceutically acceptable salt thereof.

16. The compound according to embodiment 15, wherein R¹¹ is:
i) a 5- or 6-membered heterocycle selected from:

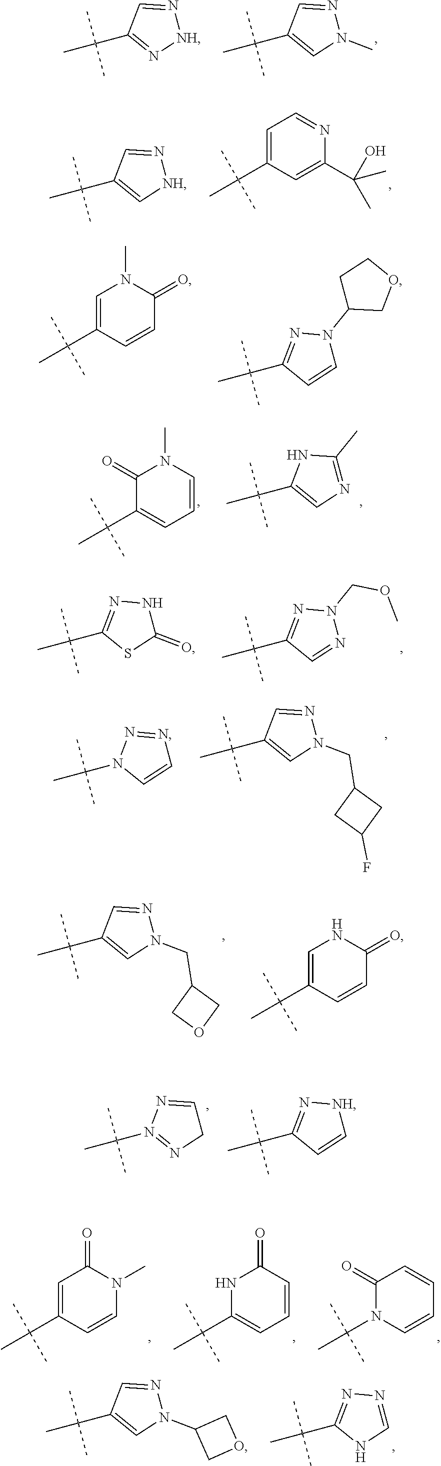

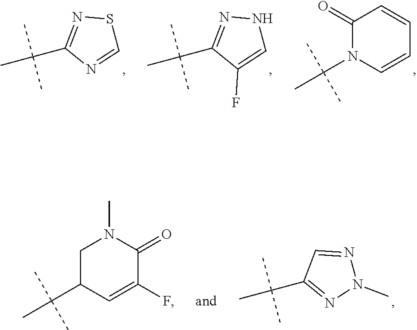

ii) a 9-membered N-containing bicyclic heterocycle selected from:

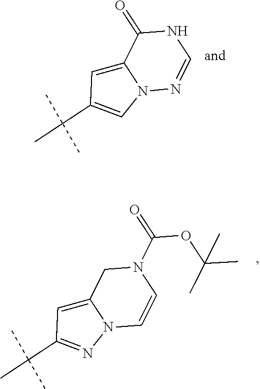

iii) cyclopropyl, cyclobutyl,
iv) Br, or
v) C(O)OCH₃, or a pharmaceutically acceptable salt thereof.

17. A compound of formula:

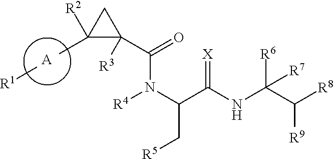

R¹ is:
   i) a 9- or 10-membered N-containing bicyclic heterocycle optionally substituted with 1 or 2 substituents independently selected from:
      C₃-C₄cycloalkyl optionally substituted with 1 or 2 halo or CH₃,
      4-membered heterocycle optionally substituted with CH₃,
      C₁-C₄alkoxy,
      C₁-C₄haloalkyl, oxo, and
      C₁-C₄alkyl optionally substituted with OCH₃, OH or C₃-C₄cycloalkyl, wherein the C₃-C₄cycloalkyl is optionally substituted with 1 or 2 halo,
   ii) NHCOR¹²,
   iii) OH, or
   iv) a 6-membered heterocycle optionally substituted with 1 or 2 substituents independently selected from: C(O)CH₃, C₁-C₄alkyl and oxo;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
A is phenyl, a 6-membered N-containing heteroaryl or thienyl, wherein the phenyl, heteroaryl or thienyl is optionally substituted with 1 or 2 substituents independently selected from halo, CF₃, CHF₂, CH₃ and cyclopropyl;
R² is H, CH₃, halo, CF₃ or CHF₂;
R³ is H and R⁴ is H, CH₃ or CD₃; or R³ and R⁴ together form —CH₂—CH₂—;
R⁵ is:
   i) a 8- or 9-membered N-containing bicyclic heterocycle optionally substituted with halo, or
   ii) a 5- or 6-membered N-containing heteroaryl optionally substituted with CH₃, CD₃, CHF₂ or OH;
X is O or S;
R⁶ and R⁷ are each CH₃ or together form a cyclopropyl;
R⁸ is H, CH₃, CO₂H or C(O)OCH₃ and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
   i) a 5- or 6-membered heterocycle optionally substituted with 1 to 4 substituents independently selected from:
      halo,
      OH
      OCH₃,
      CD₃,
      OCHF₂,
      SO₂NH₂,
      CN,
      C₁-C₄haloalkyl,
      C₃-C₅cycloalkyl,
      C(O)NH₂,
      a 4- to 6-membered heterocycle optionally substituted with oxo, oxo, and
      C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 or 2 substituents independently selected from CN, CH₃, halo, oxo, C(O)O(CH₃)₃ and OH,
   iii) C₁-C₄alkoxy,
   iv) C₃-C₅cycloalkyl,
   v) halo,
   vi) C(O)OCH₃, or
   vii) phenyl optionally substituted with 1 to 3 substituents independently selected from: halo and C₁-C₄alkyl, wherein the C₁-C₄alkyl is optionally substituted with OH; and
R¹⁴ and R¹⁵ are independently H or CH₃;
or a pharmaceutically acceptable salt thereof.

18. A compound of the formula:

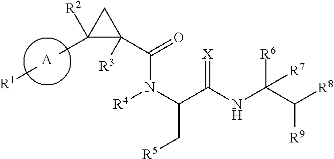

wherein
R¹ is:
   i) an 8-, 9- or 10-membered N-containing bicyclic heterocycle optionally substituted with 1 to 3 substituents independently selected from:
      C₃-C₄cycloalkyl optionally substituted with 1 or 2 halo or CH₃,
      4-membered heterocycle optionally substituted with CH₃,
      C₁-C₄alkoxy,
      C₁-C₄haloalkyl,
      oxo,
      CN,
      halo, and
      C₁-C₄alkyl optionally substituted with OCH₃, OH or C₃-C₄cycloalkyl, wherein the C₃-C₄cycloalkyl is optionally substituted with 1 or 2 halo,
   ii) NHCOR¹²,
   iii) OH, or
   iv) a 5- or 6-membered heterocycle optionally substituted with 1 or 2 substituents independently selected from: C(O)CH₃, C₁-C₄alkyl, oxo and CN;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
A is phenyl, a 6-membered N-containing heteroaryl or thienyl, wherein the phenyl, heteroaryl or thienyl is optionally substituted with 1 or 2 substituents independently selected from halo, CF₃, CHF₂, CH₃, cyclopropyl or CN, or when R¹ is an optionally substituted 8-, 9- or 10-membered N-containing bicyclic heterocycle A may be absent;
R² is H, CH₃, halo, CF₃ or CHF₂;
R³ is H and R⁴ is H, CH₃ or CD₃; or R³ and R⁴ together form —CH₂—CH₂—;
R⁵ is:
   i) a 8- or 9-membered N-containing bicyclic heterocycle optionally substituted with halo, or
   ii) a 5- or 6-membered N-containing heteroaryl optionally substituted with CH₃, CD₃, CHF₂ or OH;
X is O or S;
R⁶ and R⁷ are each CH₃ or together form a cyclopropyl;
R⁸ is H, CH₃, CO₂H or C(O)OCH₃ and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
   i) a 5- or 6-membered heterocycle optionally substituted with 1 to 4 substituents independently selected from:
      halo,
      OH,
      OCH₃,
      CD₃,
      OCHF₂,
      SO₂NH₂,
      CN,
      C₁-C₄haloalkyl,
      C₃-C₅cycloalkyl,
      C(O)NH₂,
      a 4- to 6-membered heterocycle optionally substituted with oxo,
      oxo,
      CR¹⁴R¹⁵OP(O)(OH)₂, and
      C₁-C₄alkyl, wherein the C₁-C₄alkyl is optionally substituted with OH, OCH₃,
      C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 or 2 substituents independently selected from CN, CH₃, halo, oxo, C(O)O(CH₃)₃ and OH,
   iii) C₁-C₄alkoxy,
   iv) C₃-C₅cycloalkyl,
   v) halo,
   vi) C(O)OCH₃, or
   vii) phenyl optionally substituted with 1 to 3 substituents independently selected from: halo and C₁-C₄alkyl, wherein the C₁-C₄alkyl is optionally substituted with OH; and
R¹⁴ and R¹⁵ are independently H or CH₃;
or a pharmaceutically acceptable salt thereof.

19. The compound according to embodiment 17 or embodiment 18, wherein X is O, or a pharmaceutically acceptable salt thereof.

20. The compound according to any one of embodiments 17 to 19, wherein R⁶ and R⁷ are each CH₃, or a pharmaceutically acceptable salt thereof.

21. The compound according to any one of embodiments 17 to 20, wherein R³ is H and R⁴ is H, CH₃ or CD₃, or a pharmaceutically acceptable salt thereof.

22. The compound according to any one of embodiments 17 to 20, wherein R³ and R⁴ together form —CH₂—CH₂—, or a pharmaceutically acceptable salt thereof.

23. The compound according to any one of embodiments 17 to 22, wherein R² is H or CH₃, or a pharmaceutically acceptable salt thereof.

24. The compound according to any one of embodiments 17 to 23, wherein R¹ is:
i) a 9- or 10-membered N-containing bicyclic heterocycle selected from:

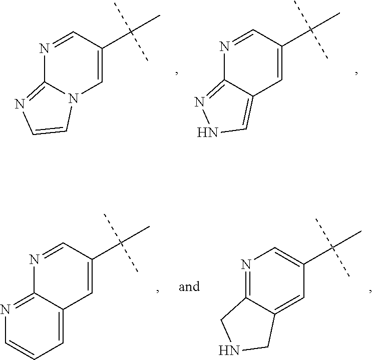

optionally substituted with 1 or 2 substituents independently selected from: C₃-C₄cycloalkyl optionally substituted with 1 or 2 halo or CH₃,
   4-membered heterocycle optionally substituted with CH₃,
   C₁-C₄alkoxy,
   C₁-C₄haloalkyl,
   oxo, and
   C₁-C₄alkyl optionally substituted with OCH₃, OH or C₃-C₄cycloalkyl, wherein the C₃-C₄cycloalkyl is optionally substituted with 1 or 2 halo,
ii) NHCOR¹², wherein R¹² is NHR¹³ or C₃-C₆cycloalkyl optionally substituted with 1 or 2 halo and R¹³ is phenyl optionally substituted with 1 or 2 halo, or
iii)

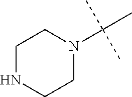

optionally substituted with C(O)CH₃, or a pharmaceutically acceptable salt thereof.

25. The compound according to embodiment 24, wherein R¹ is selected from:

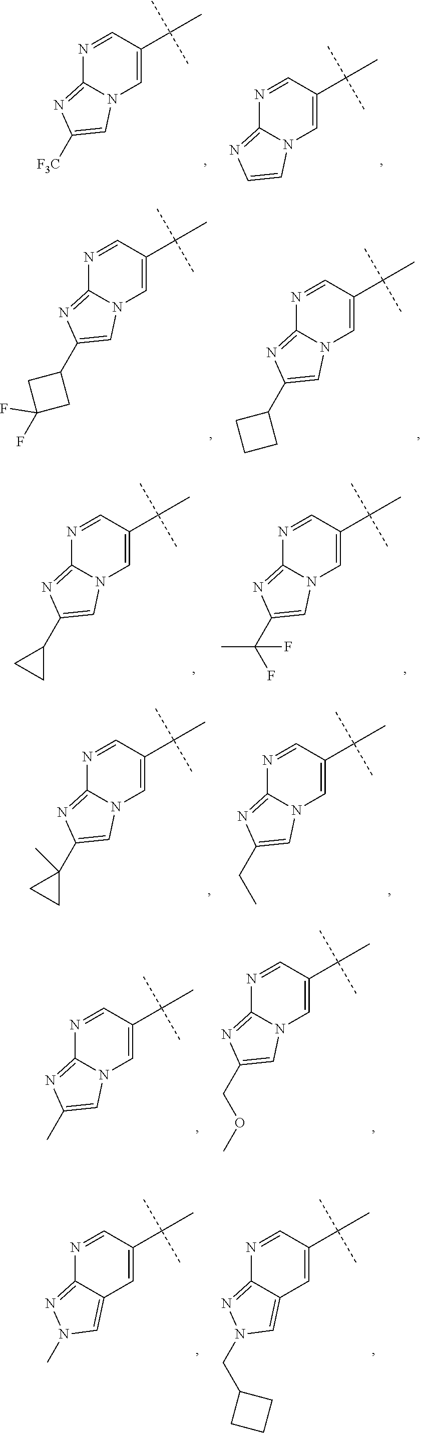

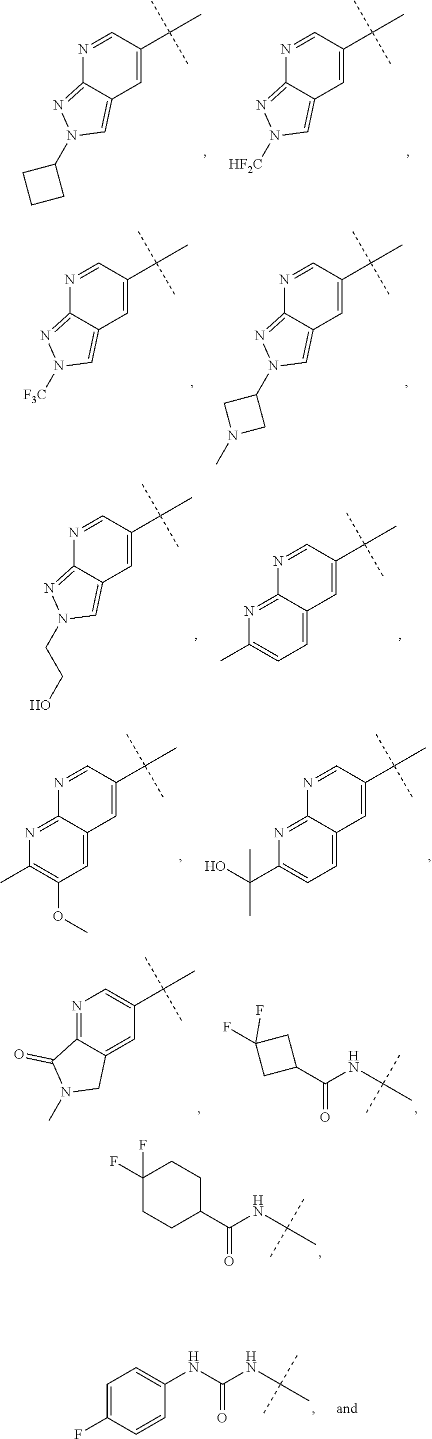

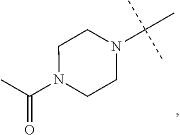

or a pharmaceutically acceptable salt thereof.

26. The compound according to any one of embodiments 17 to 25, wherein A is phenyl, pyridyl, pyrimidinyl or thienyl, wherein the phenyl, pyridyl, pyrimidinyl or thienyl is optionally substituted with a substituent selected from: F, Cl, CF₃, CHF₂ and CH₃, or a pharmaceutically acceptable salt thereof.

27. The compound according to embodiment 26, wherein A is selected from:

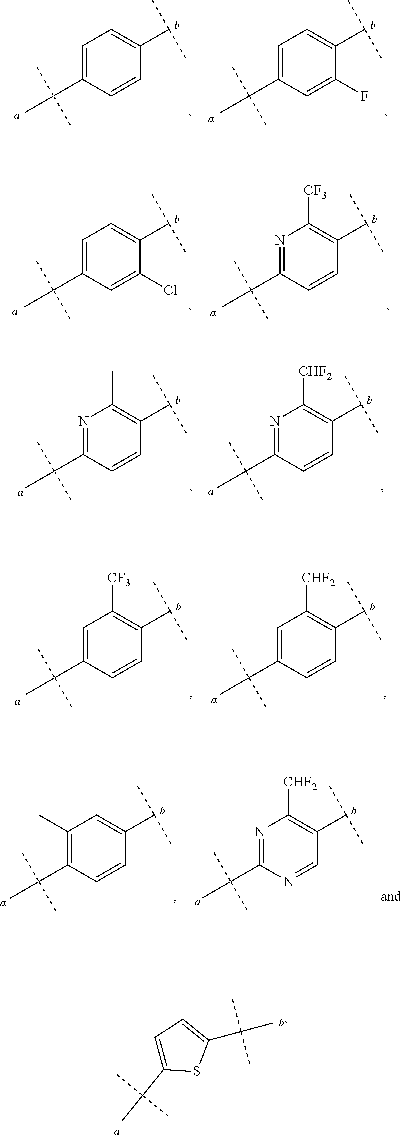

wherein a is the point of attachment to R¹ and b is the point of attachment to the cyclopropyl, or a pharmaceutically acceptable salt thereof.

28. The compound according to any one of embodiments 17 to 27, wherein R⁵ is:
i) a 9-membered N-containing bicyclic heterocycle, or
ii) a 5-membered N-containing heteroaryl substituted with CH₃, CD₃ or CHF₂,
or a pharmaceutically acceptable salt thereof.

29. The compound according to embodiment 28, wherein R⁸ is:

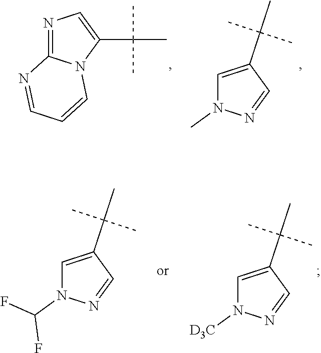

or a pharmaceutically acceptable salt thereof.

30. The compound according to any one of embodiments 17 to 29, wherein R⁸ is H, or a pharmaceutically acceptable salt thereof.

31. The compound according to any one of embodiments 17 to 30, wherein R⁹ is:
phenyl substituted with F, or
a 5- or 6-membered N-containing heteroaryl optionally substituted with R¹⁰ or with R¹⁰ and R¹¹, or a pharmaceutically acceptable salt thereof.

32. The compound according to embodiment 31, wherein R⁹ is:

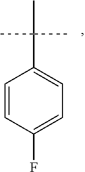

or
a 5- or 6-membered N-containing heteroaryl selected from:

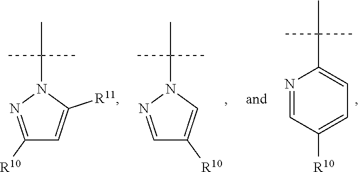

or a pharmaceutically acceptable salt thereof.

33. The compound according to embodiment 32, wherein R⁹ is:
a 5- or 6-membered N-containing heteroaryl selected from:

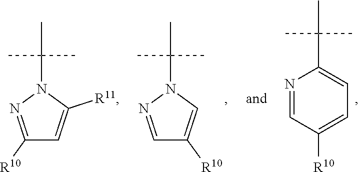

R¹⁰ is CF₃, CF₂H or F, and R¹¹ is:
   i) a 5- or 6-membered heterocycle optionally substituted with 1 to 4 substituents independently selected from:
      halo,
      OH,
      CD₃,
      OCHF₂,
      CN,
      C₁-C₄haloalkyl,
      C₃-C₅cycloalkyl,
      C(O)NH₂,
      a 4- to 6-membered heterocycle optionally substituted with oxo,
      oxo, and
      C₁-C₄alkyl, wherein said C₁-C₄ alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
   ii) a 9- or 10-membered N-containing bicyclic heterocycle optionally substituted with 1 to 2 substituents independently selected from CH₃, halo, oxo and C(O)O(CH₃)₃,
   iii) C₃-C₅cycloalkyl,
   iv) halo,
   v) C(O)OCH₃, or
   vi) phenyl optionally substituted with 1 to 3 substituents independently selected from: halo and C₁-C₄alkyl, wherein the C₁-C₄alkyl is optionally substituted with OH;
R¹⁴ and R¹⁵ are independently H or CH₃, or a pharmaceutically acceptable salt thereof.

34. The compound according to embodiment 33, wherein R¹¹ is:
i) a 5- or 6-membered heterocycle selected from:

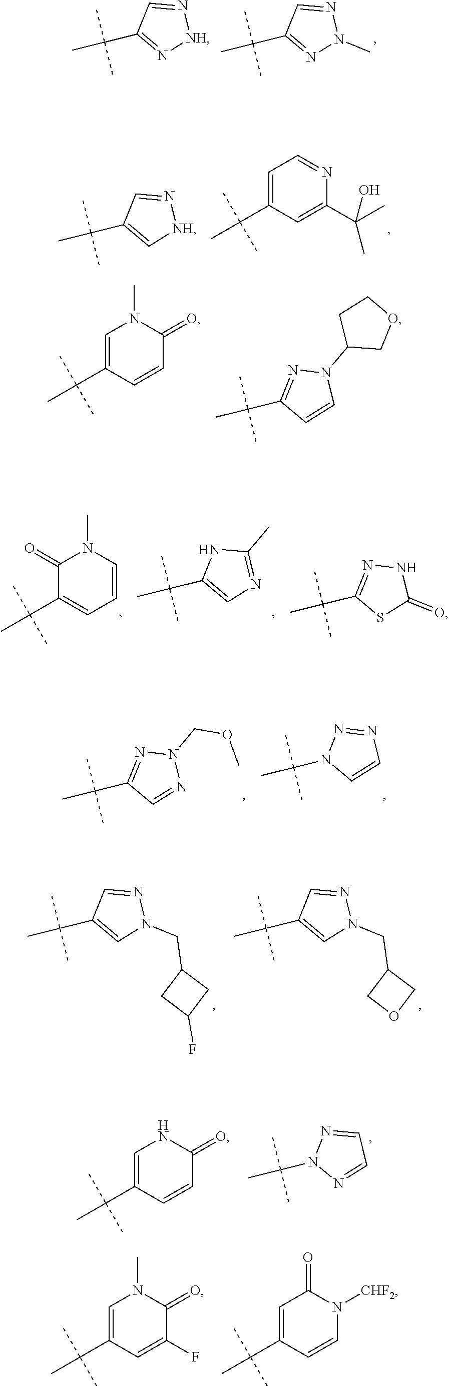

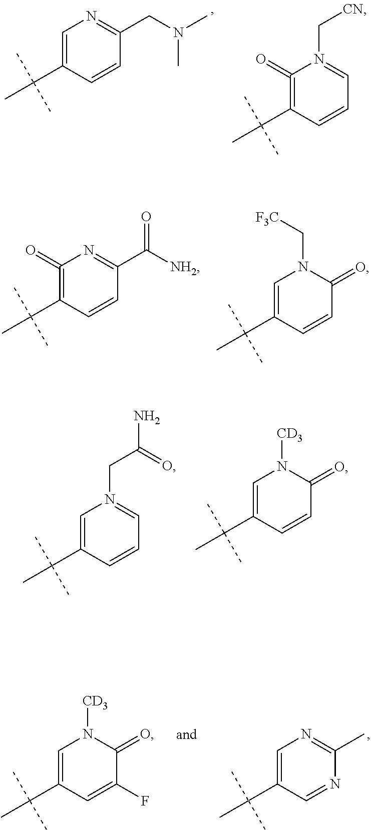

ii) a 9- or 10-membered N-containing bicyclic heterocycle selected from:

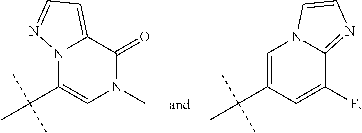

iii) cyclopropyl, or
iv) C(O)OCH₃,
or a pharmaceutically acceptable salt thereof.

In an embodiment, there is provided a pharmaceutical composition comprising a compound of any one of the above formulae, or a pharmaceutically acceptable salt thereof, and at least one of a pharmaceutically acceptable carrier, diluent or excipient. In a preferred embodiment, the pharmaceutical composition is formulated for oral administration.

In an embodiment, there is provided a method for treating type II diabetes mellitus comprising administering to the patient in need thereof a compound of any one of the above formulae, or a pharmaceutically acceptable salt thereof. In another embodiment, there is provided a method for treating type II diabetes mellitus comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In an embodiment, there is provided a method for treating obesity comprising administering to the patient in need thereof a compound of any one of the above formulae, or a pharmaceutically acceptable salt thereof. In another embodiment, there is provided a method for treating obesity comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In a further embodiment, there is provided a method for chronic weight management, a method for improving weight management or a method for providing therapeutic weight loss, comprising administering to the patient in need thereof a compound of any one of the above formulae, or a pharmaceutically acceptable salt thereof. In another embodiment, there is provided a method for chronic weight management, a method for improving weight management or a method for providing therapeutic weight loss comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In a further embodiment, there is provided a method for treating overweight with at least one weight related comorbidity, comprising administering to the patient in need thereof a compound of any one of the above formulae, or a pharmaceutically acceptable salt thereof. In another embodiment, there is provided a method for treating overweight with at least one weight related comorbidity comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided a method for treating a disorder selected from: type 1 diabetes mellitus, metabolic syndrome, dyslipidemia, hepatic steatosis associated with insulin resistance and diabetes, chronic kidney disease (CKD), atherosclerosis, non-alcoholic fatty liver disease (NAFLD), non-alcoholic steatohepatitis (NASH), diabetic kidney disease, a cognitive disorder, heart failure, high blood pressure, obstructive sleep apnea and bone frailty, comprising administering to the patient in need thereof a compound of any one of the above formulae, or a pharmaceutically acceptable salt thereof. In another embodiment, there is provided a method for treating a disorder selected from: metabolic syndrome, dyslipidemia, hepatic steatosis associated with insulin resistance and diabetes, chronic kidney disease (CKD), atherosclerosis, non-alcoholic fatty liver disease (NAFLD), non-alcoholic steatohepatitis (NASH), diabetic kidney disease, a cognitive disorder, heart failure, high blood pressure, obstructive sleep apnea and bone frailty, comprising administering to the patient in need thereof a pharmaceutical composition comprising a therapeutically effective amount of a compound of any one of the above formulae, or a pharmaceutically acceptable salt thereof, and at least one of a pharmaceutically acceptable carrier, diluent or excipient.

In an embodiment, there is provided a compound of any one of the above formulae, or a pharmaceutically acceptable salt thereof, for use in therapy.

In another embodiment, there is provided a compound of any one of the above formulae, or a pharmaceutically acceptable salt thereof, for use in the treatment of type II diabetes mellitus.

In another embodiment, there is provided a compound of any one of the above formulae, or a pharmaceutically acceptable salt thereof, for use in treating obesity.

In a further embodiment, there is provided a compound of any one of the above formulae, or a pharmaceutically acceptable salt thereof, for use in chronic weight management, improving weight management or providing therapeutic weight loss.

In a further embodiment, there is provided a compound of any one of the above formulae, or a pharmaceutically acceptable salt thereof, for use in treating overweight with at least one weight related comorbidity. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided a compound of any one of the above formulae, or a pharmaceutically acceptable salt thereof, for use in treating a disorder selected from: type 1 diabetes mellitus, metabolic syndrome, dyslipidemia, hepatic steatosis associated with insulin resistance and diabetes, CKD, atherosclerosis, NAFLD, NASH, diabetic kidney disease, a cognitive disorder, heart failure, high blood pressure, obstructive sleep apnea and bone frailty.

In an embodiment, there is provided the use of a compound of any one of the above formulae, or a pharmaceutically acceptable salt thereof, for the manufacture of a medicament for the treatment of type II diabetes mellitus.

In an embodiment, there is provided the use of a compound of any one of the above formulae, or a pharmaceutically acceptable salt thereof, for the manufacture of a medicament for the treatment of obesity.

In a further embodiment, there is provided the use of a compound of any one of the above formulae, or a pharmaceutically acceptable salt thereof, for the manufacture of a medicament for chronic weight management, improving weight management or providing therapeutic weight loss.

In a further embodiment, there is provided the use of a compound of any one of the above formulae, or a pharmaceutically acceptable salt thereof, for the manufacture of a medicament for treating overweight with at least one weight related comorbidity. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided the use of a compound of any one of the above formulae, or a pharmaceutically acceptable salt thereof, for the manufacture of a medicament for the treatment of a disorder selected from: type 1 diabetes mellitus, metabolic syndrome, dyslipidemia, hepatic steatosis associated with insulin resistance and diabetes, CKD, atherosclerosis, NAFLD, NASH, diabetic kidney disease, a cognitive disorder, heart failure, high blood pressure, obstructive sleep apnea and bone frailty.

In an embodiment, there is provided a pharmaceutical composition for treating type II diabetes mellitus comprising an effective amount of a compound of any one of the above formulae, or a pharmaceutically acceptable salt thereof.

In an embodiment, there is provided a pharmaceutical composition for treating obesity comprising an effective amount of a compound of any one of the above formulae, or a pharmaceutically acceptable salt thereof.

In a further embodiment, there is provided a pharmaceutical composition for chronic weight management, improving weight management or providing therapeutic weight loss comprising an effective amount of a compound of any one of the above formulae, or a pharmaceutically acceptable salt thereof.

In a further embodiment, there is provided a pharmaceutical composition for treating overweight with at least one weight related comorbidity comprising an effective amount of a compound of any one of the above formulae, or a pharmaceutically acceptable salt thereof. In a particular embodiment, the weight related comorbidity is selected from cancer, depression, diabetes, dyslipidemia, high blood pressure, high cholesterol, obstructive sleep apnea, osteoarthritis, and heart disease.

In a further embodiment, there is provided a pharmaceutical composition for treating a disorder selected from: type 1 diabetes mellitus, metabolic syndrome, dyslipidemia, hepatic steatosis associated with insulin resistance and diabetes, CKD, atherosclerosis, NAFLD, NASH, diabetic kidney disease, a cognitive disorder, heart failure, high blood pressure, obstructive sleep apnea and bone frailty, comprising an effective amount of a compound of any one of the above formulae, or a pharmaceutically acceptable salt thereof.

The compounds of any one of the above formulae may be used in simultaneous, separate, or sequential combination with one or more therapeutic agents. Examples of additional therapeutic agents include, but are not limited to, GLP-1 receptor agonist, a glucagon receptor agonist, a dual GLP-1-glucagon receptor agonist, metformin, a thiazolidinedione, a sulfonylurea, a dipeptidyl peptidase 4 inhibitor, a sodium-glucose cotransporter-2 (SGLT-2) inhibitor, a growth differentiation factor 15 modulator (“GDF15”), a peptide tyrosine modulator (“PYY”), a modified insulin, an amylin receptor agonist, a dual amylin-calcitonin receptor agonist and a modified urocortin-2 (UCN-2) agonist.

In a preferred embodiment, the compound of any one of the above formulae is administered orally. In a preferred embodiment, the compound of any one of the above formulae is administered once daily. In another preferred embodiment, the therapeutic use is in a human.

References to Formula IX should be understood to refer to compounds of Formula IX together with any and all sub-formulae disclosed herein, including Formulae I, II, III, IV, V, VI, VII, VIII and IX.

The term “halogen” or “halo” refers to fluorine, chlorine, bromine, or iodine.

The term “C₁-Cₙalkyl” refers to a straight, or branched chain saturated hydrocarbon containing 1 to n carbon atoms. Examples of a C₁-C₄alkyl group include, but are not limited to, methyl, ethyl, propyl, butyl, and tert-butyl.

The term “C₁-Cₙhaloalkyl” refers to a C₁-Cₙalkyl group, as defined herein, which is substituted with one or more halogen. Examples of C₁-C₄haloalkyl groups include, but are not limited to, trifluoromethyl, difluoromethyl and pentafluoroethyl.

The term “C₁-Cₙalkoxy” refers to a straight, or branched chain saturated hydrocarbon containing 1 to n carbon atoms containing a terminal “O” in the chain, i.e., —O(alkyl). Examples of C₁-C₄alkoxy groups include, but are not limited to, methoxy, ethoxy, propoxy and butoxy.

The term “C₁-Cₙhaloalkoxy” refers to a C₁-Cₙalkoxy group, as defined herein, which is substituted with one or more halogen. Examples of C₁-C₄haloalkoxy groups include, but are not limited to, difluoromethoxy and 2,2-difluoroethoxy.

The term “C₃-Cncycloalkyl” refers to a monocyclic saturated carbon ring containing between 3 and n carbon atoms.

The term “heterocycle” refers to an aromatic, saturated or partially saturated ring containing one or more heteroatoms, preferably selected from: N, S and O. An example of a 4-membered heterocycle includes, but is not limited to, oxetane. Examples of 5-membered heterocycles include, but are not limited to, pyrazole, imidazole, triazole, thiophene, oxadiazole and thiadiazole. Examples of 6-membered heterocycles include, but are not limited to, pyridine and pyridazine.

The term “N-containing bicyclic heterocycle” refers to a bicyclic aromatic, saturated or partially saturated ring comprising at least one nitrogen atom and optionally one or more other heteroatoms. An example of an 8-membered N-containing bicyclic heterocycle includes, but is not limited to, dihydropyrroloimidazole. Examples of 9-membered N-containing bicyclic heterocycles include, but are not limited to, pyrazolopyrimidine, imidazopyrimidine, dihydropyrazolopyrazine, dihydropyrrolotriazine and tetrahydropyrazolopyrazine. Examples of 10-membered N-containing bicyclic heterocycles include, but are not limited to, naphthyridine and pyridopyridazine.

The term “heteroaryl” refers to a monocyclic aromatic ring containing one or more heteroatoms, preferably selected from: N, S and O. An “N-containing heteroaryl” comprises at least one nitrogen atom but may optionally comprise one or more other heteroatoms. Examples of 5-membered N-containing heteroaryls include, but are not limited to, pyrazole, imidazole, triazole, oxadiazole and thiadiazole. Examples of 6-membered N-containing heteroaryls include, but are not limited to, pyridine and pyridazine.

The compounds of Formula IX provided herein, or a pharmaceutically acceptable salt thereof, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl refers to both C₂H₅ or C₂H₅ where from 1 to 5 hydrogens are replaced by deuterium, such as in C₂DₓH₅₋ₓ. Unless otherwise stated, when an atom is designated specifically as “D” or “deuterium”, the atom is understood to have deuterium at an abundance substantially greater than the natural abundance of deuterium, which is 0.015%.

The term “pharmaceutically acceptable salt” as used herein refers a salt of a compound of the invention considered to be acceptable for clinical and/or veterinary use. Examples of pharmaceutically acceptable salts and common methodologies for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66 (1), 1-19.

A compound herein, or a pharmaceutically acceptable salt thereof, includes all stereoisomers of the compound, for example, an enantiomer, a diastereomer (including cis- and trans-geometric isomer), the racemic form of the isomers, and other mixtures. For example, the compound herein, or a pharmaceutically acceptable salt thereof, may have one or more asymmetric centers.

The term “therapeutically effective amount” refers to the amount or dose of a compound of Formula IX, or a pharmaceutically acceptable salt thereof, which, upon single or multiple dose administration to the patient, provides the desired effect in the patient under diagnosis or treatment. The attending physician, as one skilled in the art, can readily determine an effective amount by the use of conventional techniques and by observing results obtained under analogous circumstances. Factors considered in the determination of a therapeutically effective amount or dose of a compound include: whether the compound or its salt will be administered; the co-administration of other agents, if used; the size, age, and general health of the patient; the degree of involvement or the severity of the disorder; the response of the individual patient; the mode of administration; the bioavailability characteristics of the preparation administered; the dose regimen selected; and other relevant circumstances.

As used herein, the terms “treating”, “to treat”, or “treatment”, refers to lowering, reducing, or reversing the progression or severity of an existing symptom, disorder, or condition.

As used herein, the term “patient” includes mammals. The patient is preferably human.

The term “overweight with at least one weight related comorbidity” refers to a disease or condition of being overweight and at least one weight related comorbidity. In a particular embodiment, the comorbidity is selected from cancer, depression, diabetes, dyslipidemia, high blood pressure, high cholesterol, obstructive sleep apnea, osteoarthritis, and heart disease. In one embodiment, a subject being overweight is defined as having a body mass index (BMI) of ≥25 to <30.

The compounds of Formula IX can be formulated as pharmaceutical compositions administered by any route which makes the compound bioavailable. Preferably, such compositions are for oral administration. Preferably the pharmaceutical compositions are formulated as a tablet, capsule, or a solution. The tablet, capsule, or solution can include a compound of Formula IX in an amount effective for treating a patient in need of treatment. Such pharmaceutical compositions and processes for preparing same are well known in the art (See, e.g., “Remington: The Science and Practice of Pharmacy”, A. Adejare Editor, 23rd Ed., 2020, Elsevier Science).

The compounds of the present invention, or salts thereof, may be prepared by a variety of procedures known to one of ordinary skill in the art, some of which are illustrated in the schemes, preparations, and examples below. The products of each step in the schemes below can be recovered by conventional methods well known in the art, including extraction, evaporation, precipitation, chromatography, filtration, trituration, and crystallization. In the schemes below, all substituents unless otherwise indicated, are as previously defined. The reagents and starting materials are readily available to one of ordinary skill in the art. Without limiting the scope of the invention, the following schemes, preparations, and examples are provided to further illustrate the invention. In addition, one of ordinary skill in the art appreciates that compounds of Formula IX may be prepared by using starting material or intermediate with the corresponding desired stereochemical configuration which can be prepared by one of skill in the art.

### Abbreviations:

“aq.” refers to aqueous
“BOC” or “Boc” refers to tert-butyloxycarbonyl
“n-BuOH” refers to n-butanol
“CDCl₃” refers to deuterated chloroform
“ACN” refers to acetonitrile
“DBU” refers to 1,8-diazabicyclo[5.4.0]undec-7-ene
“DCE” refers to dichloroethane
“DCM” refers to dichloromethane
“DEPBT” refers to diethyl (4-oxobenzo[d][1,2,3]triazin-3 (4H)-yl)phosphate
“DIEA” refers to N,N-diisopropylethylamine
“DMA” refers to N,N-dimethylacetamide
“DMAP” refers to N,N-dimethylpyridine-4-amine
“DMEA” refers to N,N-dimethylethylamine
“DME” refers to 1,2-dimethoxyethane
“DMEM” refers to Dulbecco's Modified Eagle's Medium
“DMF” refers to N,N-dimethylformamide
“DMSO” refers to dimethyl sulfoxide
“dppf” refers to 1,1′-bis(diphenylphosphino)ferrocene
“EDCI” refers to 1-(3-dimethylaminopropyl)-3-ethylcarbodiimide hydrochloride
“ee” refers to enantiomeric excess
“ES-MS” refers to electrospray mass spectrometry
“EtOAc” refers to ethyl acetate
“EtOH” refers to ethanol
“Et₂O” refers to diethyl ether
“eq” refers to equivalents
“FA” refers to formic acid
“h” refers to hours
“HATU” refers to 1-[Bis(dimethylamino)methylene]-1H-1,2,3-triazolo[4,5-b]pyridinium 3-oxid hexafluorophosphate
“HEPES” refers to (4-(2-hydroxyethyl)-1-piperazineethanesulfonic acid
“HFIP” refers to 1,1,1,3,3,3-hexafluoroisopropanol
“¹H-NMR” refers to proton nuclear magnetic resonance
“Prep-HPLC” refers to preparative high-performance liquid chromatography
“HOAc” refers to acetic acid
“IPA” refers to isopropanol
“IPAm” refers to isopropylamine
“KOAc” refers to potassium acetate
“LiHMDS” refers to lithium bis(trimethylsilyl) amide
“MeOH” refers to methanol
“mPa” refers to millipascal
“min” refers to minutes
“MTBE” refers to methyl tert-butyl ether
“m/z” refers to mass-to-charge ratio
“NaHMDS” refers to sodium bis(trimethylsilyl) amide
“NCS” refers to N-chlorosuccinimide
“NH₄OAc” refers to ammonium acetate
“NMP” refers to N-methyl pyrrolidine-2-one
“PDA” refers to photodiode array detection
“PE” refers petroleum ether
“Ph” refers to phenyl
“PPh₃” refers to triphenylphosphine
“ppm” refers to parts per million
“Prep” refers to preparation
“psi” refers to pounds per square inch
“Raney® Ni” refers to Raney® Nickel
“RT” refers to room temperature
“Rt” refers to retention time
“Rf” refers to retention factor
“sat.” refers to saturated
“SFC” refers to supercritical fluid chromatography
“SPE” refers to solid phase extraction
“TBAI” refers to tetrabutylammonium iodide
“tBu” refers to tert-butyl
“TEA” refers to triethylamine
“Temp” refers to temperature
“TFA” refers to trifluoroacetic acid
“THF” refers to tetrahydrofuran
“2-MeTHF” refers to 2-methyltetrahydrofuran
“wt %” refers to percentage by weight

### Catalyst Abbreviations:

“(A-taPhos)₂PdCl₂” refers to Bis(di-tert-butyl (4-dimethylaminophenyl)phosphine)dichloropalladium (II) (CAS #887919-35-9)
“BrettPhos Pd G3” refers to [(2-Di-cyclohexylphosphino-3,6-dimethoxy-2′,4′,6′-triisopropyl-1,1′-biphenyl)-2-(2′-amino-1,1′-biphenyl)]palladium (II) methanesulfonate (CAS #1470372-59-8)
“cataCXium® A Pd G3” refers to mesylate[(di(1-adamantyl)-n-butylphosphine)-2-(2′-amino-1,1′-biphenyl)]palladium (II) (CAS #1651823-59-4)
“cataCXium Pd G4” refers to [di(adamantan-1-yl)(butyl)phosphine](methanesulfonato-κO) [2′-(methylamino)-2-biphenylyl]palladium (CAS #2230788-67-5) or mesylate[(di(1-adamantyl)-n-butylphosphine)-2-(2′-MeNH-1,1′-biphenyl)]palladium (II)
“CPhos Pd G3” refers to methanesulfonato (2-dicyclohexylphosphino-2′,6′-bis(dimethylamino)-1,1′-biphenyl) (2′-amino-1,1′-biphenyl-2-yl) palladium (II) (CAS #1447963-73-6)
“(dtbbpy)NiCl₂” refers to [4,4-bis(1,1-dimethylethyl)-2,2′-bipyridine] nickel(II) dichloride (CAS #1034901-50-2)
“[Ir(dF(CF₃)ppy)₂(dtbpy)]PF₆” refers [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium (III) hexafluorophosphate (CAS #870987-63-6)
“[Ir(OMe)(1,5-cod)]₂” refers to (1,5-Cyclooctadiene)(methoxy)iridium(I) dimer (CAS #12148-71-9)
“Pd(amphos)Cl₂” refers to bis(di-tert-butyl(4-dimethylaminophenyl)phosphine)dichloropalladium(II) (CAS #887919-35-9)
“Pd₂(dba)₃” refers to tris(dibenzylideneacetone)dipalladium (CAS #51364-51-3)
“PdCl₂(dtbpf)” refers to 1,1′-bis(di-tert-butylphosphino)ferrocene palladium dichloride (CAS #95408-45-0)
“Pd(PPh₃)₄” refers to tetrakis(triphenylphosphine)palladium(0) (CAS #14221-01-3)
“Pd(dppf)Cl₂” or “PdCl₂(dppf)” refers to [1,1-bis(diphenylphosphino)ferrocene]dichloropalladium (II) (CAS #72287-26-4)
“Pd(dppf)Cl₂·CH₂Cl₂” or “PdCl₂(dppf)·CH₂Cl₂” refers to [1,1′-bis(diphenylphosphino)ferrocene]dichloropalladium (II), complex with dichloromethane (CAS #95464 May 4)
“Rh-COD-[(R)-MaxPhos]-BF₄” refers to [((R)-tert-butylmethylphosphino)(di-tert-butylphosphino)amine](1,5-cyclooctadiene)rhodium(I) tetrafluoroborate (CAS #1263077-53-7)
“(Ru(OAc)₂[(R)-BINAP]” refers to diacetato[(R)-(+)-2,2′-bis(diphenylphosphino)-1,1′-binaphthyl]ruthenium(II) (CAS #325146-81-4)
“Ru-(R)-Pheox” refers to Ruthenium-tetrakis(acetonitrile)[2-[(4R)-4,5-dihydro-4-phenyl-2-oxazolyl-κN3]phenyl-κC]-hexafluorophosphate (CAS #1421679-43-7)
“XantPhos Pd G4” refers to (SP-4-3)-[[5-(diphenylphosphino)-9,9-dimethyl-9H-xanthen-4-yl]diphenylphosphine-κP](methanesulfonato-κO)[2′-(methylamino-κN)[1,1′-biphenyl]-2-yl-κC]-Palladium (CAS #1621274-19-8)
“XPhos” refers to 2-dicyclohexylphosphino-2′,4′,6′-triisopropylbiphenyl (CAS #564483-18-7)
“XPhos Pd G3” refers to (2-dicyclohexylphosphino-2′,4′,6′-triisopropyl-1,1′-biphenyl)[2-(2′-amino-1,1′-biphenyl)]palladium (II) methanesulfonate (CAS #1445085-55-1)

In the following schemes, R¹, R², R³, R⁴, R⁵, R⁶, R⁷, R⁸, R^{8′}, R9, R¹⁰, R¹¹, A and Z are as defined in Formula IX.

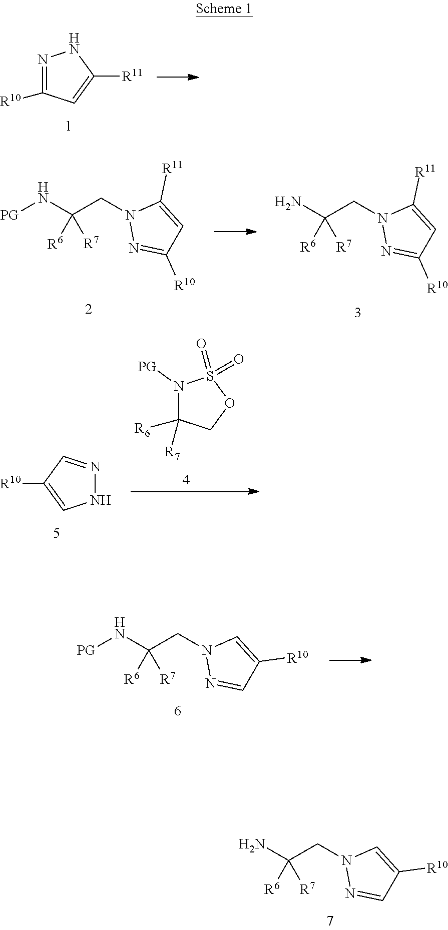

Scheme 1 shows the preparation of intermediates 3 and 7, which are useful for the preparation of compounds of the present invention. Imidazole intermediates 1 and 5 are reacted with oxathiazolidine intermediate 4 using a carbonate base such as Cs₂CO₃ in a solvent such as DMA at elevated temperature to give intermediates 2 and 6, respectively. The nitrogen protecting groups (represented as “PG” in the above scheme) of intermediates 2 and 6 are then removed under conditions known to the person skilled in the art, for example Boc-protecting groups can be removed under acidic conditions (e.g. stirring the Boc-protected intermediate in HCl in 1,4-dioxane at RT) to give 3 and 7, respectively. The same steps may also be used to prepare intermediates having a single R¹⁰ substituent at position 3.

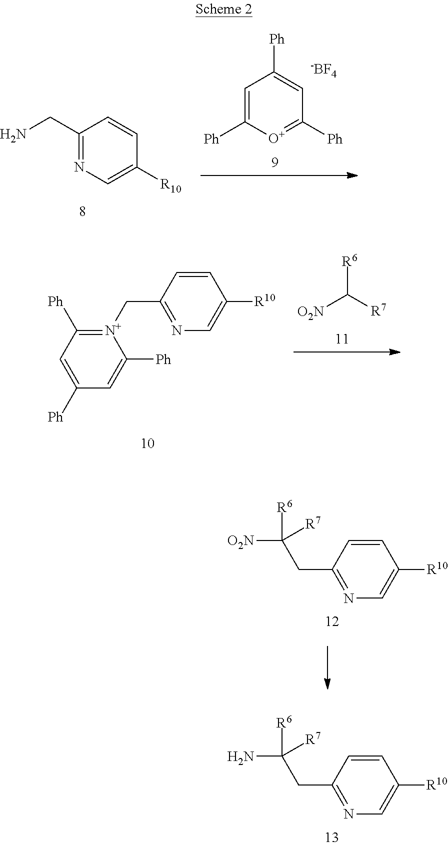

Scheme 2 shows the preparation of intermediate 13, which is useful for the preparation of compounds of the present invention. Amine 8 is first reacted with 2,4,6-triphenylpyrylium tetrafluoroborate 9 in a suitable solvent such as DCM to give intermediate 10. Nitro intermediate 11 is reacted with an alkoxide base such as sodium methoxide in an alcohol solvent such as MeOH, and the resulting product is reacted with intermediate 10 in a solvent such as DMSO at elevated temperature to give 12. The nitro group is then reduced under conditions known to the person skilled in the art, for example using a Raney® nickel catalyst and H₂ to give amine 13.

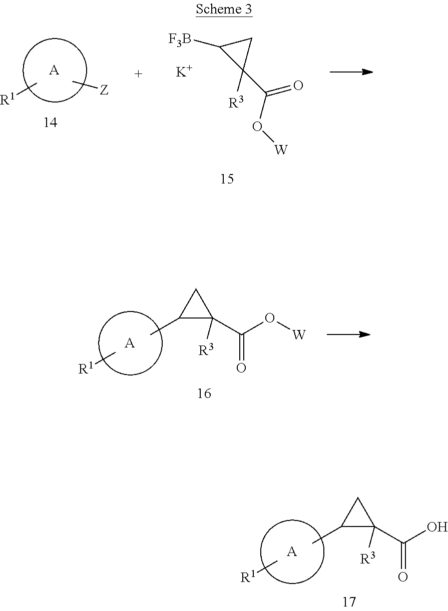

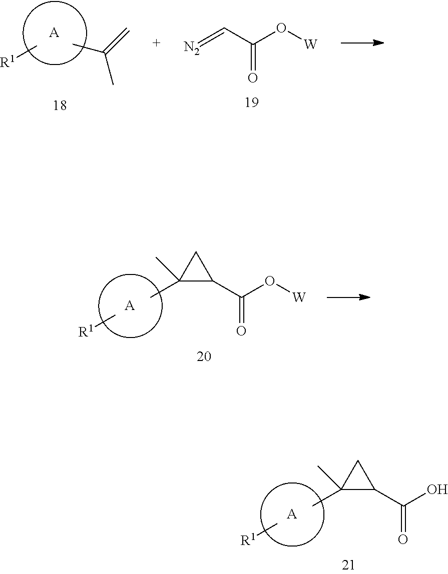

Scheme 3 shows the preparation of intermediates 17 and 21, which are useful for the preparation of compounds of the present invention. Aryl/heteroaryl halide 14 (where “Z” is a halogen such as bromine) is coupled with trifluoroborate intermediate 15 (where “W” is an alkyl group such as methyl, ethyl, or tert-butyl) using a palladium catalyst such as cataCXium Pd G4 and a carbonate base such as Cs₂CO₃ in an aqueous solvent mix such as toluene/water at elevated temperature to give 16. Alternatively, vinyl intermediate 18 is reacted with diazo compound 19 (where “W” is an alkyl group such as methyl, ethyl, or tert-butyl) in the presence of rhodium (II) acetate in a solvent such as THF to give 20. Ester intermediates 16 and 20 are then converted to acids 17 and 21, respectively, under conditions known to the person skilled in the art. For example tert-butyl esters are converted to acids under acidic conditions (e.g. TFA in chlorinated solvent such as DCM), and methyl and ethyl esters are hydrolyzed under aqueous basic conditions (e.g. LiOH in aqueous THF, at RT or at elevated temperature).

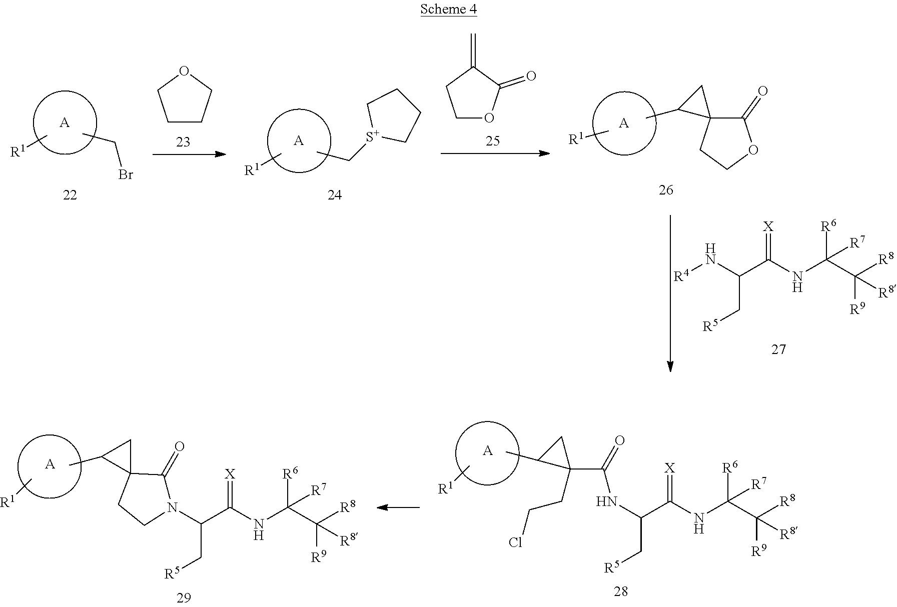

Scheme 4 shows the preparation of compounds of the present invention. Alkyl bromide 22 is reacted with tetrahydrothiophene 23 in a solvent such as acetone or ACN, optionally with addition of silver tetrafluoroborate to give 24, followed by reaction with 3-methylenedihydrofuran-2(3H)-one 25 using a base such as lithium bis(trimethylsilyl) amide in a solvent such as THF to give intermediate 26. Intermediate 26 is reacted with SOCl₂, benzyltriethylammonium chloride, and BF₃ etherate in a solvent such as DCE, then reacted with amine intermediate 27 using an organic base such as TEA in a solvent such as DCM to give intermediate 28. Intermediate 28 is then cyclized to spirocyclic compound of Formula IX using a carbonate base such as Cs₂CO₃ in a solvent such as NMP.

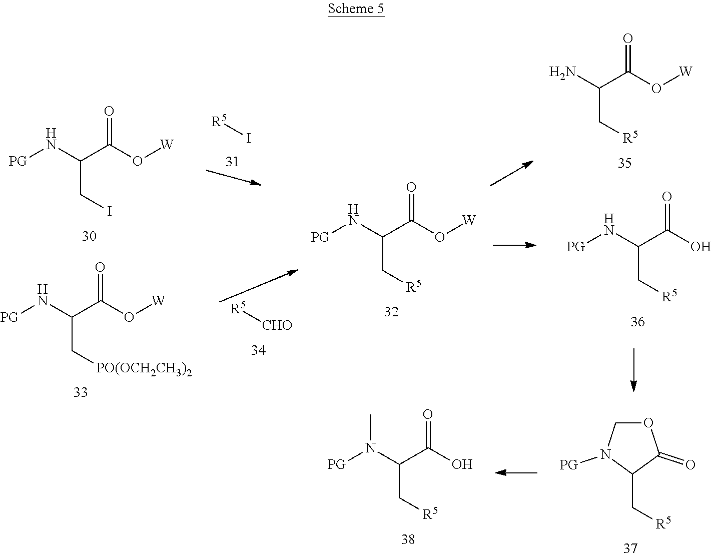

Scheme 5 shows the preparation of amino acid derivatives which are useful for the preparation of compounds of the present invention. Alkyl iodide 30 is reacted with aryl/heteroaryl iodide 31 (where “W” is an alkyl group such as methyl, ethyl, or tert-butyl and “PG” is a protecting group such as Boc or benzyloxycarbonyl) using pyridine-2,6-bis(carboximidamide) dihydrochloride, nickel(II) chloride, and zinc in a solvent such as DMA to give 32. Alternatively, phosphoryl compound 33 (where “W” and “PG” are as described above) undergoes a Horner-Wadsworth-Emmons reaction with aldehyde 34 using a base such as DBU in a solvent such as DCM, then the resulting olefin is hydrogenated to give 32. Olefin reduction conditions are known to a person of skill in the art, and can be achieved asymmetrically by hydrogenation with a catalyst such as Rh-COD-[(R)-MaxPhos]-BF₄ in a fluorinated alkane solvent (e.g. 1,1,1,3,3,3-hexafluoroisopropanol and 2,2,2-trifluoroethanol) at elevated temperature. If orthogonal protecting groups are chosen for “PG” and “W”, protected amino acid 32 can be deprotected under conditions known to a person of skill in the art to either to amine 35 or acid 36. For example, where “W” is methyl or ethyl, aqueous LiOH in a solvent such as TFH converts 32 to acid 36, and where “PG” is Boc 36 could be prepared from 32 under acidic conditions (e.g. using a solution of HCl in 1,4-dioxane). Acid 36 can be converted to N-methyl amino acid 38 by first reacting it with paraformaldehyde using p-toluene sulfonic acid in a solvent such as toluene at elevated temperature to give cyclic compound 37, which is then opened with TFA, HCl (as a solution in 1,4-dioxane), and triethylsilane in DCE to give 38.

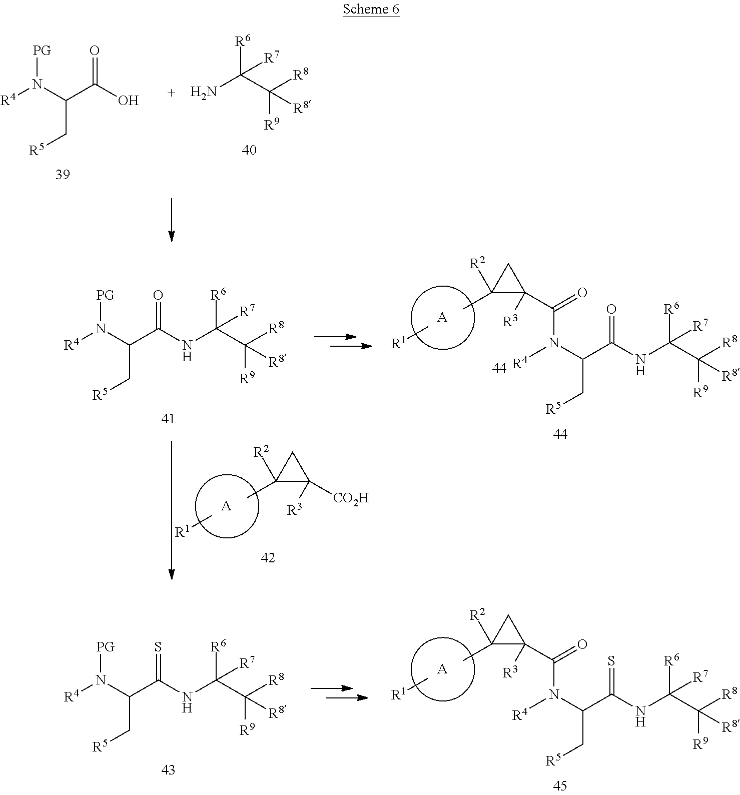

Scheme 6 shows the preparation of compounds of the present invention, which can be prepared from various amine and acid intermediates using amide coupling and deprotection chemistry in a varied order of steps. Reaction conditions used to couple amines with carboxylic acids are known to a person of skill in the art. This can include reacting a mixture of the amine and carboxylic acid with HATU and an organic base such as DIEA in a solvent such as DMF, DMA, or DMSO at temperatures from 0° C. to RT. Reaction conditions used to deprotect Boc or benzyloxycarbonyl protected amines are known to a person of skill in the art, which can include reacting a Boc-protected amine with HCl in 1,4-dioxane or reacting a benzyloxycarbonyl-protected amine with H₂ in the presence of palladium on carbon catalyst in an alcohol solvent. Reaction conditions used to convert carboxylic esters to carboxylic acids are also known to those skilled in the art. For example, methyl and ethyl esters can be hydrolyzed to carboxylic acids using an aqueous carbonate base at elevated temperature. Amine 39 (where “PG” is a protecting group such as Boc or benzyloxycarbonyl) is coupled with 40 to give 41, which is then deprotected and coupled with 42 to give 44. Alternatively, 41 is reacted with Lawesson's reagent in an organic solvent such as THF at elevated temperature to give 43, which is then deprotected and coupled with 42 to give 45.

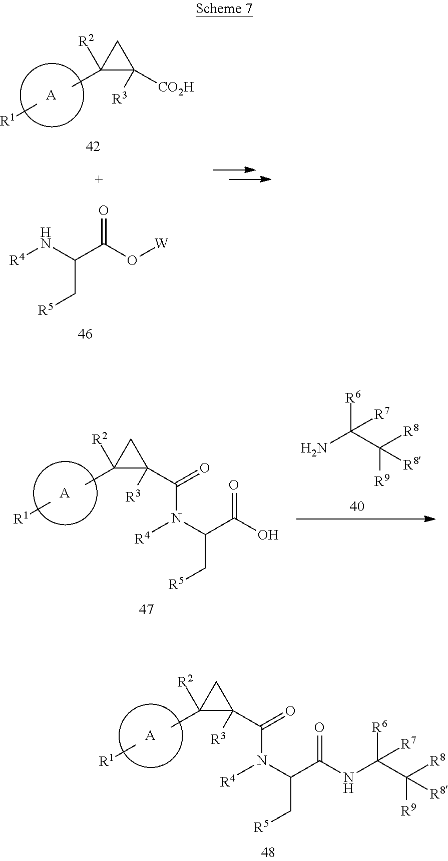

Scheme 7 shows the preparation of compounds of the present invention starting by coupling acid 42 with amine 46, and then the ester is hydrolyzed to give 47. Coupling acid 47 with amine 40 then gives compound 48. Suitable coupling and ester hydrolysis conditions are described in Scheme 6.

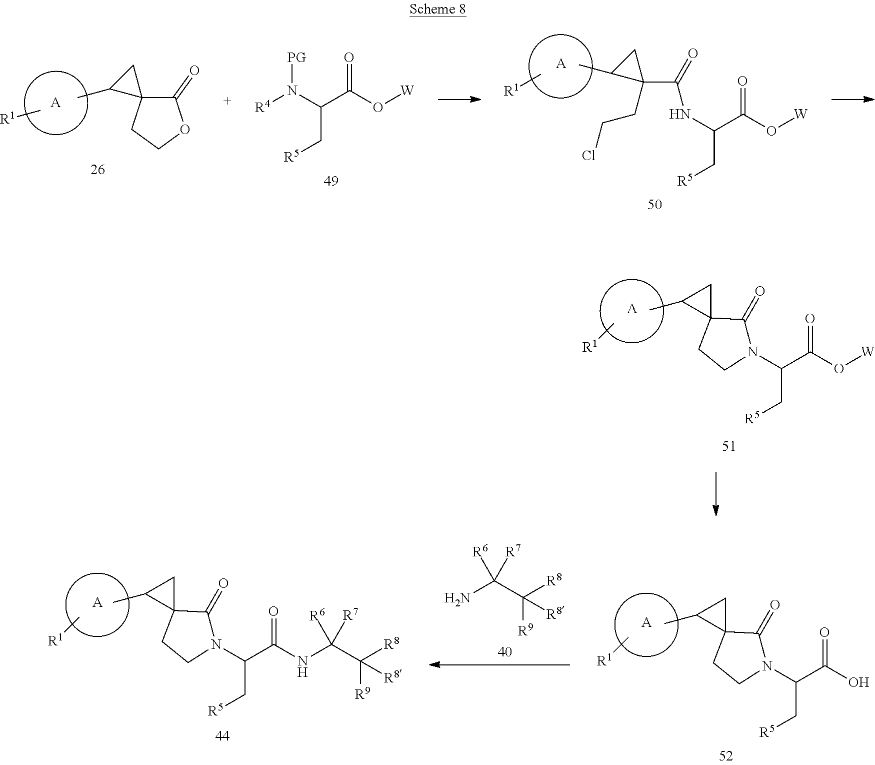

Scheme 8 shows the preparation of compounds of the present invention starting with intermediate 26, which is reacted with SOCl₂, benzyltriethylammonium chloride, and BF₃ etherate in a solvent such as DCE, then reacted with amine intermediate 49 using an organic base such as TEA in a solvent such as THF and/or DMA to give intermediate 50. Intermediate 50 is then cyclized to spirocyclic compound 51 using a carbonate base such as Cs₂CO₃ in a solvent such as NMP or THF. The ester 51 is hydrolyzed using aq. LiOH in tert-butanol to give 52, which is then coupled with amine 40 under amide coupling conditions known to one skilled in the art, such as DEPBT and N-methylmorpholine in a solvent such as DMA to give compound

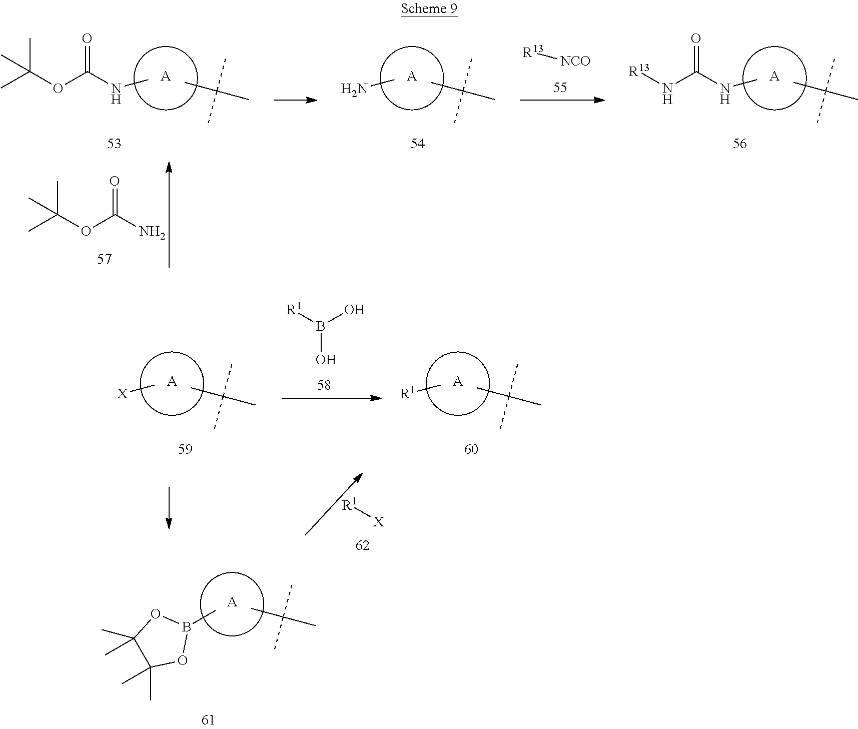

In Schemes 3, 4, 6, 7, and 8, R¹ can be introduced onto ring A at various points in the synthetic routes if ring A bears a halogen by metal catalyzed cross-coupling reactions with coupling partner intermediates bearing R¹ as defined in Formula IX, or groups which can be converted to R¹ in subsequent steps. Scheme 9 shows these synthetic steps. For example, if ring A bears a halogen (intermediate 59 where “Z” is a halogen) it can be coupled with boronic acids (e.g. intermediate 58 or analogous boronic ester where R¹ is a heterocycle as defined in Formula IX) using a palladium catalyst (e.g. Pd(dppf)Cl₂) and a carbonate base (e.g. Cs₂CO₃) in an organic/aqueous solvent mix (e.g. 1,4-dioxane/water) to give 60. An alternate approach is to react 59 with bis(pinacolato)diboron using a palladium catalyst such as Pd(dppf)Cl₂ and a base such as KOAc in a solvent such as 1,4-dioxane at elevated temperature to give boronate intermediate 61. Intermediate 61 can then be coupled with 62 (where “Z” is a halogen) under palladium coupling conditions (e.g. conditions described for the coupling of 58 and 59).

Alternatively, tert-butyl carbamate 57 can be coupled with 59 using a palladium catalyst such as Pd₂(dba)₃ with XPhos, and carbonate base (e.g. Cs₂CO₃) in an organic solvent such as 1,4-dioxane at elevated temperature to give 53. Boc deprotection using conditions described above (e.g. HCl in MeOH) gives an —NH₂ group on ring A (intermediate 54), which can then be reacted with an isocyanate (e.g. intermediate 55) in the presence of an organic base (e.g. TEA) in a solvent such as DCM to give 56.

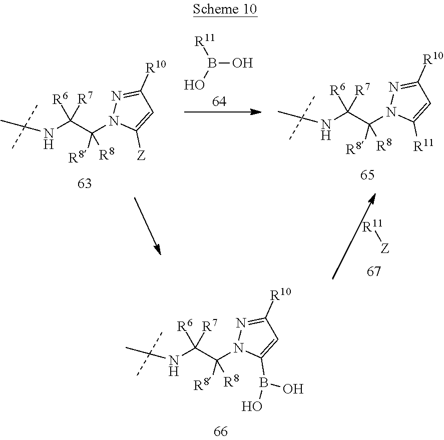

Scheme 10 shows the introduction of R¹¹ (where R¹¹ is an aromatic ring as defined in Formula IX) onto pyrazole 63, where “Z” is a halogen and 63 represents a compound also represented in Scheme 1, 4, 6, 7, or 8. Compound 63 can be coupled with boronic acid 64 (or an analogous boronic ester) using a palladium catalyst such as Pd(dppf)Cl₂ and a carbonate base such as Cs₂CO₃ in an organic/aqueous solvent such as 1,4-dioxane and water at elevated temperature to give compound 65. Alternatively, halide 63 is reacted with tetrahydroxydiboron, XPhos Pd G3, and KOAc in MeOH and DCE to give boronic acid 66, which is then coupled with 67 (where “Z” is a halogen) using a palladium catalyst such as Pd (dtbpf)Cl₂ or XPhos Pd G3, and a carbonate base such as Na₂CO₃ or K₂CO₃ in an organic/aqueous solvent mix such as 1,4-dioxane/water or 1,4-dioxane/ethanol/water at elevated temperature to give compound 65.

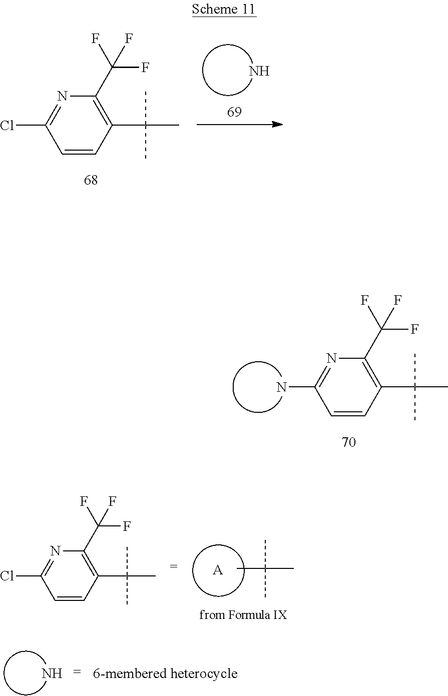

In Schemes 3, 4, 6, 7, and 8, 6-membered heterocycles can be introduced as R¹ onto ring A at various points in the synthetic routes if ring A is a 2-chloro-substituted pyridine. Scheme 11 shows 2-chloropyridine analog 68 undergoing a S_{N}Ar reaction with 6-membered heterocycle 69 in an organic solvent such as DMSO at elevated temperature to give compound 70.

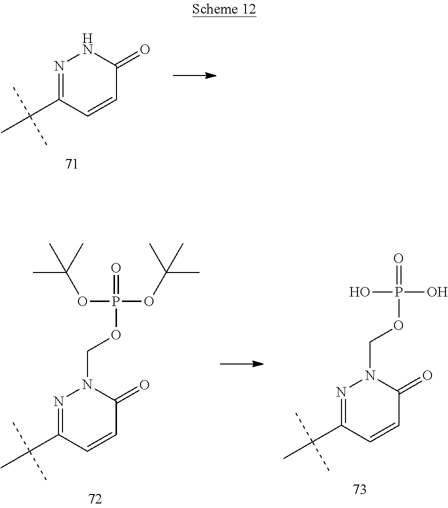

Scheme 12 shows the introduction of a methyl-dihydrogenphosphate group onto a R¹¹ group (as defined in Formula IX) comprising a 6-oxopyridazine ring. The 6-oxopyridazine compound 71 is reacted with di-tert-butyl(chloromethyl)phosphate using lithium tert-butoxide and sodium iodide in a solvent such as THF to give 72, which is then reacted with TFA in a solvent such as DCM to give 73.

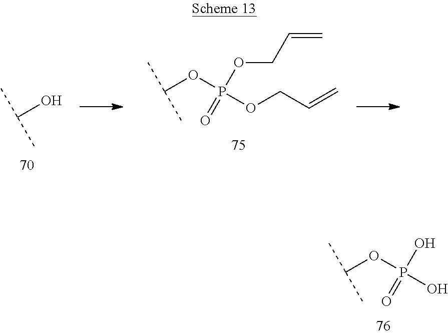

Compounds of the present invention bearing an aliphatic alcohol substituent can be converted to dihydrogen phosphates (—OP(O)(OH)₂) as shown in Scheme 13. Alcohol 74 is reacted with diallyl-N,N-diisopropylphosphoramidite using tetrazole in a solvent such as ACN at 0° C. and the mixture is treated with aq. H₂O₂ or anhydrous tert-butyl hydroperoxide to give diallyl phosphate 75, which is then converted to phosphate 76 using phenylsilane and Pd(PPh₃)₄ in a solvent such as DCM at 0° C.

### Preparation 1

### 1-(3-(Trifluoromethyl)-1H-pyrazol-5-yl)pyridin-2(1H)-one

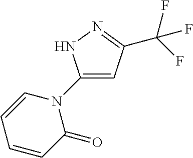 [SMILES: O=c1ccccn1-c1cc(C(F)(F)F)n[nH]1]

A mixture of 3-bromo-5-(trifluoromethyl)-1H-pyrazole (1.0 g, 4.7 mmol), pyridin-2(1H)-one (0.44 g, 4.7 mmol), K₂CO₃ (1.3 g, 9.3 mmol), copper (I) iodide (0.44 g, 2.3 mmol), (1S,2S)-N1,N2-dimethylcyclohexane-1,2-diamine (0.33 g, 2.3 mmol) was purged with N₂. 1,4-Dioxane (10 mL) was added, and the resulting mixture was stirred at 100° C. overnight. The reaction mixture was concentrated, and the residue was purified by reversed phase flash chromatography using a gradient of 20-100% ACN/10 mM aq. NH₄HCO₃, pH=10 (with 5% MeOH) to obtain the title compound (0.21 g, 20%) as a dark green solid. ES-MS m/z 230 (M+H).

### Preparations 2 and 3

### 1-(3-(Trifluoromethyl)-1H-pyrazol-5-yl)-1H-1,2,3-triazole

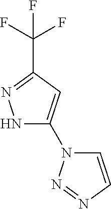 [SMILES: FC(F)(F)c1cc(-n2ccnn2)[nH]n1]

and 2-(3-(Trifluoromethyl)-1H-pyrazol-5-yl)-2H-1,2,3-triazole

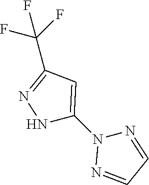 [SMILES: FC(F)(F)c1cc(-n2nccn2)[nH]n1]

A mixture of 2H-1,2,3-triazole (193 mg, 2.79 mmol), copper (I) iodide (111 mg, 0.582 mmol) and Cs₂CO₃ (1.36 g, 4.19 mmol) was purged with argon. 1,4-dioxane (9.304 mL), N,N′-dimethylethylenediamine (150 μL, 1.40 mmol) and 3-bromo-5-(trifluoromethyl)-1H-pyrazole (500 mg, 2.33 mmol) were added and the resulting mixture was stirred for 2 min at RT. After stirring at 120° C. for 4 days in a sealed 20 mL vial, most of the solvent evaporated to give a paste. MeOH (15 mL) was added, and the mixture was filtered. The collected residue was washed with MeOH (3×15 mL). The filtrate was concentrated under reduced pressure. The residue was purified by reversed phase flash chromatography using a gradient of 0-35% ACN/10 mM aq. NH₄HCO₃, pH=10 (with 5% MeOH) over 15 min to obtain the first eluting isomer, 1-(3-(trifluoromethyl)-1H-pyrazol-5-yl)-1H-1,2,3-triazole (51.3 mg, 11%), as a pale yellow solid. ES-MS (m/z) 204.0 (M+H). The second eluting isomer, 2-(3-(trifluoromethyl)-1H-pyrazol-5-yl)-2H-1,2,3-triazole (99.2 mg, 21%), was also obtained as a yellow solid. ES-MS m/z 204 (M+H).

### Preparation 4

### tert-Butyl (1-(5-bromo-3-(trifluoromethyl)-1H-pyrazol-1-yl)-2-methylpropan-2-yl) carbamate

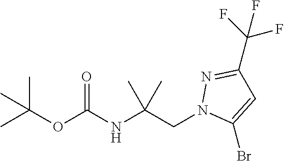 [SMILES: CC(C)(Cn1nc(C(F)(F)F)cc1Br)NC(=O)OC(C)(C)C]

A mixture of 3-bromo-5-(trifluoromethyl)-1H-pyrazole (4.11 g, 19.1 mmol), tert-butyl 4,4-dimethyl-1,2,3-oxathiazolidine-3-carboxylate 2,2-dioxide (4.00 g, 15.9 mmol) and Cs₂CO₃ (10.4 g, 31.8 mmol) was purged with N₂. DMA (32 mL) was added, and the resulting mixture was stirred at 95° C. for 6 h. The reaction mixture was cooled and poured into water (350 mL) and EtOAc (75 mL). The layers were separated, and the aq. layer was extracted with EtOAc (4×40 mL). The organic layers were combined, washed with water (40 mL) then sat. aq. NaCl (50 mL), dried over Na₂SO₄, filtered, and concentrated under reduced pressure. The residue was purified by silica gel chromatography using a gradient of 0-10% MTBE in heptane to obtain the second eluting isomer as the title compound (4.11 g, 67%). ES-MS m/z 330,332 (M-tBu+H).

### Preparation 5

### Methyl 1-(2-((tert-butoxycarbonyl)amino)-2-methylpropyl)-3-(trifluoromethyl)-1H-pyrazole-5-carboxylate

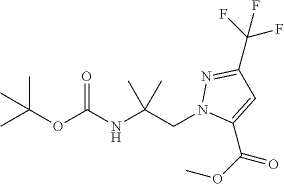 [SMILES: COC(=O)c1cc(C(F)(F)F)nn1CC(C)(C)NC(=O)OC(C)(C)C]

The title compound was prepared as described in Preparation 4 using methyl 5-(trifluoromethyl)-1H-pyrazole-3-carboxylate (stirring at 90° C. for 30 min) and isolated as the first eluting isomer (major regioisomer) when purified by silica gel chromatography with 0-15% MTBE in heptane. ES-MS m/z 310 (M-tBu+H).

### Preparation 6

### tert-Butyl (2-methyl-1-(4-(trifluoromethyl)-1H-pyrazol-1-yl)propan-2-yl)carbamate

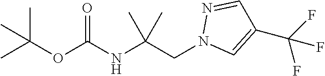 [SMILES: CC(C)(Cn1cc(C(F)(F)F)cn1)NC(=O)OC(C)(C)C]

A mixture of tert-butyl 4,4-dimethyl-1,2,3-oxathiazolidine-3-carboxylate 2,2-dioxide (30.0 g, 119 mmol), 4-(trifluoromethyl)-1H-pyrazole (24.4 g, 179 mmol) and Cs₂CO₃ (77.8 g, 239 mmol) in DMA (199 mL) was stirred at 90° C. overnight with vigorous stirring. The mixture was cooled to RT. Water (100 mL) and EtOAc (100 mL) were added, followed by dropwise addition of 1M aq. HCl (298 mL, 298 mmol). The layers were separated, and the aq. layer (approximately pH 2-3) was extracted with EtOAc (2×150 mL). The organic layers were combined, dried over Na₂SO₄, filtered, and concentrated. The residue was purified by silica gel chromatography using a gradient of 0-25% MTBE in cyclohexane to obtain the title compound (26.8 g, 73%) as a white solid. ES-MS m/z 252 (M-tBu+H).

The compounds in the following table were prepared as described in Preparation 1 using the appropriate pyrazole. Reactants can be added in different orders or in differing equivalency, and purification methods were adjusted to suit the compounds. Such variances would be apparent to one skilled in the art.

TABLE 1

| Preparation | Chemical Name | Structure | ES-MS m/z |
|---|---|---|---|
| 7^{a} | tert-Butyl (2-methyl-1-(3- (trifluoromethyl)-1H-pyrazol-1- yl)propan-2-yl)carbamate | 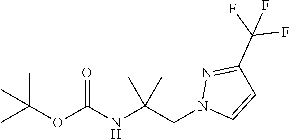 [SMILES: CC(C)(Cn1ccc(C(F)(F)F)n1)NC(=O)OC(C)(C)C] | 252 (M + H) |
| 8^{b} | tert-Butyl (1-(5-(1H-1,2,3-triazol- 1-yl)-3-(trifluoromethyl)-1H- pyrazol-1-yl)-2-methylpropan-2- yl)carbamate | 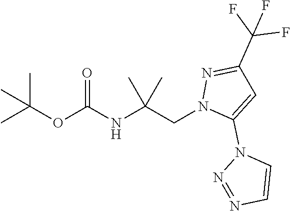 [SMILES: CC(C)(Cn1nc(C(F)(F)F)cc1-n1ccnn1)NC(=O)OC(C)(C)C] | 375 (M + H) |
| 9^{c} | tert-Butyl (2-methyl-1-(5-(2- oxopyridin-1(2H)-yl)-3- (trifluoromethyl)-1H-pyrazol-1- yl)propan-2-yl)carbamate | 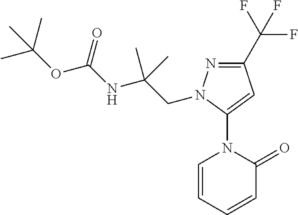 [SMILES: CC(C)(Cn1nc(C(F)(F)F)cc1-n1ccccc1=O)NC(=O)OC(C)(C)C] | 301 (M − BOC + H) |
| 10^{d} | tert-Butyl (1-(5-(2H-1,2,3-triazol- 2-yl)-3-(trifluoromethyl)-1H- pyrazol-1-yl)-2-methylpropan-2- yl)carbamate | 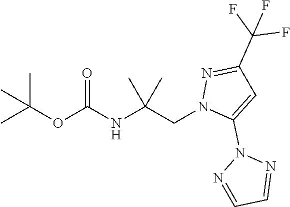 [SMILES: CC(C)(Cn1nc(C(F)(F)F)cc1-n1nccn1)NC(=O)OC(C)(C)C] | 319 (M − tBu + H) |

^{a}first eluting isomer (silica, gradient 0 to 20% MTBE/heptane), major regioisomer
^{b}second eluting isomer (silica, gradient 0 to 50% MTBE/heptane)
^{c}second eluting isomer (silica, gradient 0 to 35% MTBE/CHX), minor regioisomer
^{d}~1:4 mixture of regioisomers by high pH LCMS, favoring the title compound after purification (silica, gradient 0 to 30% MTBE/heptane)

### Preparation 11

### 1-(2-((tert-Butoxycarbonyl)amino)-2-methylpropyl)-3-(trifluoromethyl)-1H-pyrazole-5-carboxylic acid

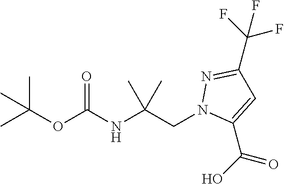 [SMILES: CC(C)(Cn1nc(C(F)(F)F)cc1C(=O)O)NC(=O)OC(C)(C)C]

A mixture of methyl 1-(2-((tert-butoxycarbonyl)amino)-2-methylpropyl)-3-(trifluoromethyl)-1H-pyrazole-5-carboxylate (221 mg, 603 μmol), THF (3 mL) and 2M aq. LiOH·H₂O (1.81 mL, 3.62 mmol) was stirred at 50° C. overnight in a sealed 20 mL vial. The reaction mixture was cooled to RT and treated with 4M HCl in 1,4-dioxane (905 μL, 3.62 mmol). After stirring for 5 min, the mixture was concentrated under a stream of N₂ to obtain a quantitative yield of the title compound as a white solid. ES-MS m/z 350 m/z (M−H).

### Preparation 12

### tert-Butyl (1-(5-carbamoyl-3-(trifluoromethyl)-1H-pyrazol-1-yl)-2-methylpropan-2-yl)carbamate

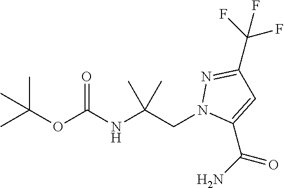 [SMILES: CC(C)(Cn1nc(C(F)(F)F)cc1C(N)=O)NC(=O)OC(C)(C)C]

A mixture of 1-(2-((tert-butoxycarbonyl)amino)-2-methylpropyl)-3-(trifluoromethyl)-1H-pyrazole-5-carboxylic acid (300 mg, 854 μmol), 1H-benzo[d][1,2,3]triazol-1-ol, ammonia salt (195 mg, 1.28 mmol), EDCI (246 mg, 1.28 mmol), THF (4.3 mL) and DIEA (0.44 mL, 2.56 mmol) was stirred at RT for 5 days in sealed 40 mL vial. The reaction mixture was treated with additional 1H-benzo[d][1,2,3]triazol-1-ol, ammonia salt (195 mg, 1.28 mmol), EDCI (246 mg, 1.28 mmol), DIEA (0.44 mL, 2.56 mmol) and THF (4.3 mL). The resulting mixture was stirred vigorously at RT for 2 days. The mixture was diluted with water (15 mL) and extracted with EtOAc (2×20 mL). The organic layers were combined, washed with sat. aq. NaCl (15 mL), dried over Na₂SO₄, filtered, and concentrated under reduced pressure to obtain a quantitative yield of the title compound as a pale pinkish-orange gel. ES-MS (m z) 251.2 (M-BOC+H).

### Preparation 13

### tert-Butyl (1-(5-(4H-1,2,4-triazol-3-yl)-3-(trifluoromethyl)-1H-pyrazol-1-yl)-2-methylpropan-2-yl)carbamate

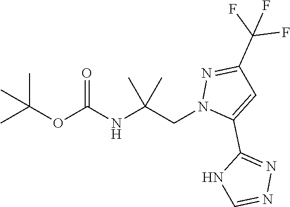 [SMILES: CC(C)(Cn1nc(C(F)(F)F)cc1-c1nnc[nH]1)NC(=O)OC(C)(C)C]

A mixture of tert-butyl (1-(5-carbamoyl-3-(trifluoromethyl)-1H-pyrazol-1-yl)-2-methylpropan-2-yl)carbamate (299 mg, 854 μmol) in dry toluene (4.3 mL) was treated with 1,1-dimethoxy-N,N-dimethyl-methanamine (341 μL, 2.56 mmol). The resulting mixture was stirred at 50° C. for 1 h in a sealed 20 mL vial then concentrated under reduced pressure to obtain a quantitative yield of tert-butyl (1-(5-(((dimethylamino) methylene) carbamoyl)-3-(trifluoromethyl)-1H-pyrazol-1-yl)-2-methylpropan-2-yl)carbamate as a yellow gel. ES/MS (m z) 406.2 m/z (M+H). HOAc (3.4 mL, 58 mmol) and hydrazine H₂O (208 μL, 4.27 mmol) were added, and the mixture was stirred at RT for 30 min. The resulting pale-yellow solution was concentrated under steam of N₂. The residue was partitioned between EtOAc (15 mL) and sat. aq. NaHCO₃ (10 mL), and the aq. layer was extracted with EtOAc (3×15 mL). The organic layers were combined, washed with sat. aq. NaCl, dried over Na₂SO₄, and concentrated under reduced pressure to obtain a quantitative yield of the title compound as a pale-yellow gel. ES-MS m z 319.2 (M-tBu+H).

### Preparation 14

### tert-Butyl (1-(5-(2-(methoxymethyl)-2H-1,2,3-triazol-4-yl)-3-(trifluoromethyl)-1H-pyrazol-1-yl)-2-methylpropan-2-yl)carbamate

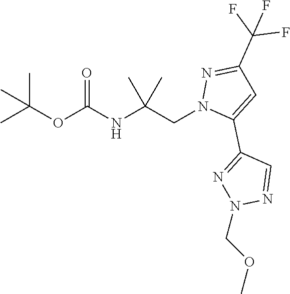 [SMILES: COCn1ncc(-c2cc(C(F)(F)F)nn2CC(C)(C)NC(=O)OC(C)(C)C)n1]

A mixture of tert-butyl (1-(5-bromo-3-(trifluoromethyl)-1H-pyrazol-1-yl)-2-methylpropan-2-yl)carbamate (1.50 g, 3.88 mmol), 2-(methoxymethyl)-4-(4,4,5,5-tetramethyl-1,3,2-dioxaborolan-2-yl)-2H-1,2,3-triazole (1.39 g, 5.83 mmol), and K₃PO₄ (2.47 g, 11.7 mmol) in DME (15.5 mL) was sparged with argon for 5 min. Pd(PPh₃)₄ (898 mg, 777 μmol) was added, and argon degassing was continued for 3 min. The resulting mixture was stirred at 95° C. overnight. The reaction mixture was cooled to RT and partitioned between water (30 mL) and EtOAc (50 mL). The aq. layer was extracted with EtOAc (3×50 mL). The organic layers were combined, washed with water (15 mL) then sat. aq. NaCl (15 mL), dried over Na₂SO₄, filtered through a pad of diatomaceous earth, and concentrated under reduced pressure. The residue was purified by silica gel chromatography using a gradient of 0-30% (3:1 EtOAc:EtOH) in heptane to obtain the title compound (0.914 g, 55%) as a yellow solid. ES-MS m/z 363.0 (M-tBu+H).

### Preparation 15

### tert-Butyl (2-methyl-1-(5-(1-methyl-6-oxo-1,6-dihydropyridin-3-yl)-3-(trifluoromethyl)-1H-pyrazol-1-yl)propan-2-yl)carbamate

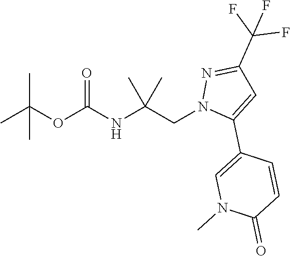 [SMILES: Cn1cc(-c2cc(C(F)(F)F)nn2CC(C)(C)NC(=O)OC(C)(C)C)ccc1=O]

A mixture of 1-methyl-5-(4,4,5,5-tetramethyl-1,3,2-dioxaborolan-2-yl)pyridin-2(1H)-one (3.04 g, 12.9 mmol), tert-butyl (1-(5-bromo-3-(trifluoromethyl)-1H-pyrazol-1-yl)-2-methylpropan-2-yl)carbamate (5.00 g, 12.9 mmol), Cs₂CO₃ (8.44 g, 25.9 mmol) and Pd(dppf)Cl₂ (947 mg, 1.29 mmol), 1,4-dioxane (78.5 mL) and water (7.85 mL) was sparged with N₂ for 5 min. The resulting mixture was stirred at 60° C. for 1 h. Another portion of 1-methyl-5-(4,4,5,5-tetramethyl-1,3,2-dioxaborolan-2-yl)pyridin-2(1H)-one (1.3 g, 5.53 mmol) was added, and the mixture was stirred at 80° C. overnight. The reaction mixture was cooled to RT, diluted with DCM, and washed with water. The organic phase was dried over MgSO₄ and concentrated under reduced pressure. The residue was purified by silica gel chromatography using a gradient of 30-100% EtOAc in cyclohexane. Crystallization from 20% EtOAc: 80% cyclohexane, followed by vacuum filtration gave the title compound (2.77 g, 52%) as a white solid. ES-MS m/z 359.0 (M-tBu+H).

The compounds in the following table were prepared as described in Preparation 15 using the appropriate commercially available boron ester or boronic acid. Varying reaction temperatures, bases and purification techniques can be used, which would be apparent to one skilled in the art.

TABLE 2

| | | | ES-MS |
|---|---|---|---|
| Prep | Chemical Name | Structure | m/z |
| 16^{a} | tert-Butyl (1-(5-cyclopropyl-3- (trifluoromethyl)-1H-pyrazol-1- yl)-2-methylpropan-2- yl)carbamate | 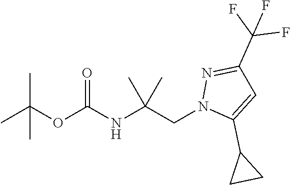 [SMILES: CC(C)(Cn1nc(C(F)(F)F)cc1C1CC1)NC(=O)OC(C)(C)C] | 348.2 (M + H) |
| 17^{b} | tert-Butyl (1-(5-(2- methoxypyridin-3-yl)-3- (trifluoromethyl)-1H-pyrazol-1- yl)-2-methylpropan-2- yl)carbamate | 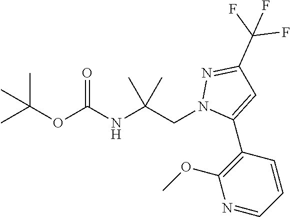 [SMILES: COc1ncccc1-c1cc(C(F)(F)F)nn1CC(C)(C)NC(=O)OC(C)(C)C] | 415.2 (M + H) |
| 18^{b} | tert-Butyl (1-(5-(6- methoxypyridin-2-yl)-3- (trifluoromethyl)-1H-pyrazol-1- yl)-2-methylpropan-2- yl)carbamate | 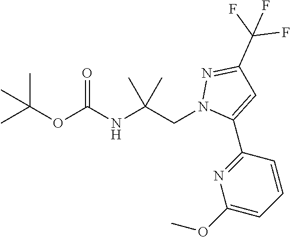 [SMILES: COc1cccc(-c2cc(C(F)(F)F)nn2CC(C)(C)NC(=O)OC(C)(C)C)n1] | 415.2 (M + H) |

^{a}used 3 eq cyclopropylboronic acid and K₂CO₃, heated at 90° C., extracted with EtOAc.
^{b}used K₂CO₃ (3 eq), heated at 95° C., extracted with 1:1 EtOAc: toluene (no further purification).

### Preparation 18a

### tert-Butyl (1-(5-cyclobutyl-3-(trifluoromethyl)-1H-pyrazol-1-yl)-2-methylpropan-2-yl)carbamate

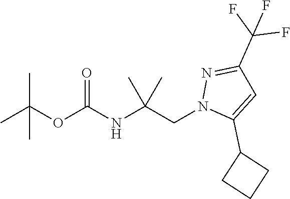 [SMILES: CC(C)(Cn1nc(C(F)(F)F)cc1C1CCC1)NC(=O)OC(C)(C)C]

A solution of tert-butyl (1-(5-bromo-3-(trifluoromethyl)-1H-pyrazol-1-yl)-2-methylpropan-2-yl)carbamate (97 mg, 0.25 mmol), [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium (III) hexafluorophosphate or [Ir(dF(CF₃)ppy)₂(dtbpy)]PF₆ (CAS #870987-63-6, 2.8 mg, 2.5 μmol), [4,4-bis(1,1-dimethylethyl)-2,2′-bipyridine] nickel(II) dichloride, or (dtbbpy) NiCl₂ (CAS #1034901-50-2, 5.0 mg, 13 μmol) and tris(trimethylsilyl) silane (78 μL, 0.25 mmol) in DME was sparged with N₂ for 5 min. 2,6-lutidine (0.15 mL, 1.3 mmol) and bromocyclobutane (50 mg, 0.37 mmol) were added, and the resulting mixture was irradiated with A465 nm light in a photochemical reactor overnight. The reaction mixture was passed through a syringe filter and purified by reversed phase prep-HPLC (Kinetex® EVO C18, 30×100 mm, 5 μm, 85 mL/min) using a gradient of 50-86% ACN in 10 mM aq. NH₄HCO₃, pH=10 (with 5% MeOH) over 6 min to obtain the title compound (20 mg, 22%) as a yellow oil. ES-MS m/z 362.0 (M+H).

### Preparation 19

### (1-(2-((tert-Butoxycarbonyl)amino)-2-methylpropyl)-3-(trifluoromethyl)-1H-pyrazol-5-yl)boronic acid

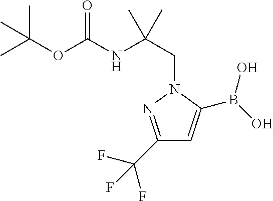 [SMILES: CC(C)(Cn1nc(C(F)(F)F)cc1B(O)O)NC(=O)OC(C)(C)C]

In a glovebox, a solution of tert-butyl (1-(5-bromo-3-(trifluoromethyl)-1H-pyrazol-1-yl)-2-methylpropan-2-yl)carbamate (1.0 g, 2.5 mmol) in MeOH (30 mL) was treated with tetrahydroxydiboron (1.1 g, 13 mmol), a solution of XPhos Pd G3 (CAS #1445085-55-1, 0.22 g, 0.25 mmol) in DCE (3 mL), and KOAc (1.0 g, 10 mmol). The resulting mixture was stirred at 40° C. for 16 h under N₂. The reaction was repeated on the same scale, and the reaction solutions were combined, adjusted pH to 6 with FA and filtered. The filtrate was concentrated. The residue was purified by silica gel chromatography using a gradient of 0-4% (0.1% FA/MeOH) in DCM to obtain the title compound (1.45 g, 80%) as a brown-oil. ES-MS m/z 352.3 (M+H).

### Preparation 20

### tert-Butyl (2-methyl-1-(5-(2-methyl-1H-imidazol-5-yl)-3-(trifluoromethyl)-1H-pyrazol-1-yl)propan-2-yl)carbamate

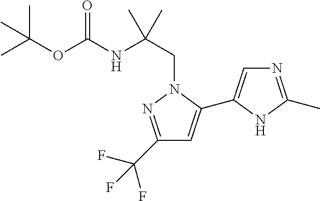 [SMILES: Cc1ncc(-c2cc(C(F)(F)F)nn2CC(C)(C)NC(=O)OC(C)(C)C)[nH]1]

To a solution (1-(2-((tert-butoxycarbonyl)amino)-2-methylpropyl)-3-(trifluoromethyl)-1H-pyrazol-5-yl) boronic acid (500 mg, 1.40 mmol), 5-bromo-2-methyl-1H-imidazole (562 mg, 3.49 mmol) in 1,4-dioxane (10 mL) and water (2 mL) was added K₂CO₃ (579 mg, 4.19 mmol) and 1,1′-bis(di-tert-butylphosphino)ferrocene palladium dichloride or PdCl₂(dtbpf) (CAS #95408-45-0, 182 mg, 279 μmol) at RT. The resulting mixture was stirred at 100° C. for 12 h under N₂, then concentrated under reduced pressure. The residue was purified by silica gel chromatography using a gradient of 0-60% EtOAc in PE to obtain the title compound (140 mg, 21%) as a yellow solid. ES-MS m/z 388.7 (M+H).

### Preparation 21

### tert-Butyl (1-(5-(5-methoxy-1,3,4-thiadiazol-2-yl)-3-(trifluoromethyl)-1H-pyrazol-1-yl)-2-methylpropan-2-yl)carbamate

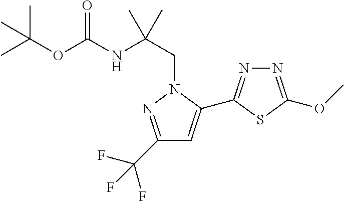 [SMILES: COc1nnc(-c2cc(C(F)(F)F)nn2CC(C)(C)NC(=O)OC(C)(C)C)s1]

A solution of 2-bromo-5-methoxy-1,3,4-thiadiazole (526 mg, 2.56 mmol), (1-(2-((tert-butoxycarbonyl)amino)-2-methylpropyl)-3-(trifluoromethyl)-1H-pyrazol-5-yl) boronic acid (1.00 g, 2.56 mmol, ˜90% purity) and Na₂CO₃ (543 mg, 5.13 mmol) in 1,4-dioxane (10 mL), EtOH (10 mL) and water (1 mL) was treated with XPhos Pd G3 (CAS #1445085-55-1, 325 mg, 384 μmol). After stirring at 90° C. for 2 h under N₂, the reaction mixture was cooled and diluted with water (20 mL). The aq. layer was extracted with EtOAc (2×30 mL). The organic layers were combined, washed with sat. aq. NaCl (3×15 mL), dried over Na₂SO₄, filtered, and concentrated under reduced pressure. The residue was purified by silica gel chromatography using a gradient of 0-40% EtOAc in PE to obtain the title compound (170 mg, 14%) as a yellow solid. ES-MS m/z 422.0 (M+H).

### Preparation 22

### 1-(5-Bromo-3-(trifluoromethyl)-1H-pyrazol-1-yl)-2-methylpropan-2-amine hydrochloride

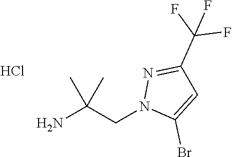

A solution of tert-butyl (1-(5-bromo-3-(trifluoromethyl)-1H-pyrazol-1-yl)-2-methylpropan-2-yl)carbamate (5.00 g, 12.9 mmol) in MeOH (12.9 mL) was treated with 4M HCl in 1,4-dioxane (12.9 mL, 51.8 mmol). After stirring for 2 h at RT, the mixture was concentrated to obtain the title compound (4.145 g, 99%) as a white solid. ES-MS (m z, ⁷⁹Br/⁸¹Br) 286.0/288.0 (M+H).

The compounds in the following table were prepared as described in Preparation 22 from the appropriate tert-butyl carbamate. Varying reaction times (2-43 h), eq of HCl 4-18 eq), and co-solvents (MeOH, EtOH, EtOAc, 1,4-dioxane) can be used, which would be apparent to one skilled in the art.

TABLE 3

| Prep | Chemical Name | Structure | ES-MS m/z |
|---|---|---|---|
| 23ª | 2-Methyl-1-(4-(trifluoromethyl)- 1H-pyrazol-1-yl)propan-2-amine hydrochloride | 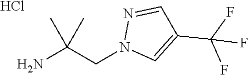 | 208.1 (M + H) |
| 24^{b} | 2-Methyl-1-(3-(trifluoromethyl)- 1H-pyrazol-1-yl)propan-2-amine hydrochloride | 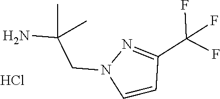 | 208.1 (M + H) |
| 25^{c} | 1-(5-(1H-1,2,3-Triazol-1-yl)-3- (trifluoromethyl)-1H-pyrazol-1- yl)-2-methylpropan-2-amine hydrochloride | 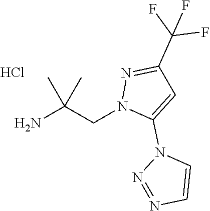 [SMILES: Cl ; CC(C)(N)Cn1nc(C(F)(F)F)cc1-n1ccnn1] | 275.2 (M + H) |
| 26^{d} | 1-(5-(2H-1,2,3-Triazol-2-yl)-3- (trifluoromethyl)-1H-pyrazol-1- yl)-2-methylpropan-2-amine hydrochloride | 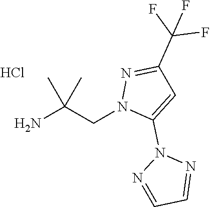 [SMILES: Cl ; CC(C)(N)Cn1nc(C(F)(F)F)cc1-n1nccn1] | 275.2 (M + H) |
| 27^{e} | 1-(1-(2-Amino-2-methylpropyl)-3- (trifluoromethyl)-1H-pyrazol-5- yl)pyridin-2(1H)-one hydrochloride | 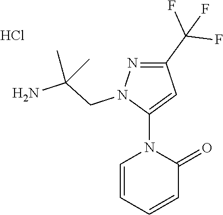 [SMILES: Cl ; CC(C)(N)Cn1nc(C(F)(F)F)cc1-n1ccccc1=O] | 301.0 (M + H) |
| 28^{c} | Methyl 1-(2-amino-2- methylpropyl)-3-(trifluoromethyl)- 1H-pyrazole-5-carboxylate hydrochloride | 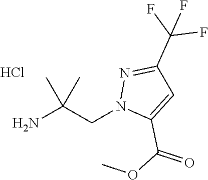 [SMILES: Cl ; COC(=O)c1cc(C(F)(F)F)nn1CC(C)(C)N] | 266.2 (M + H) |
| 29^{c} | 1-(5-(4H-1,2,4-Triazol-3-yl)-3- (trifluoromethyl)-1H-pyrazol-1- yl)-2-methylpropan-2-amine hydrochloride | 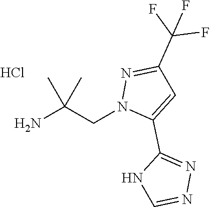 [SMILES: Cl ; CC(C)(N)Cn1nc(C(F)(F)F)cc1-c1nnc[nH]1] | 275.2 (M + H) |
| 30^{f} | 5-(1-(2-Amino-2-methylpropyl)-3- (trifluoromethyl)-1H-pyrazol-5- yl)-1-methylpyridin-2(1H)-one hydrochloride | 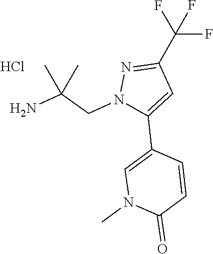 [SMILES: Cl ; Cn1cc(-c2cc(C(F)(F)F)nn2CC(C)(C)N)ccc1=O] | 315.0 (M + H) |
| 31^{c} | 1-(5-Cyclopropyl-3- (trifluoromethyl)-1H-pyrazol-1- yl)-2-methylpropan-2-amine hydrochloride | 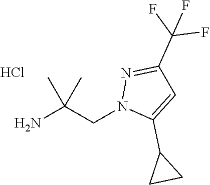 [SMILES: Cl ; CC(C)(N)Cn1nc(C(F)(F)F)cc1C1CC1] | 248.2 (M + H) |
| 32^{g} | 1-(5-Cyclobutyl-3- (trifluoromethyl)-1H-pyrazol-1- yl)-2-methylpropan-2-amine hydrochloride | 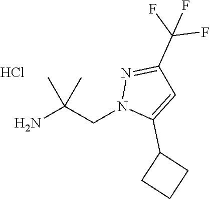 [SMILES: Cl ; CC(C)(N)Cn1nc(C(F)(F)F)cc1C1CCC1] | 262.0 (M + H) |
| 33^{h} | 2-Methyl-1-(5-(2-methyl-1H- imidazol-5-yl)-3-(trifluoromethyl)- 1H-pyrazol-1-yl)propan-2-amine hydrochloride | 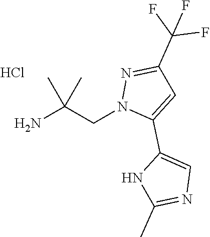 [SMILES: Cl ; Cc1ncc(-c2cc(C(F)(F)F)nn2CC(C)(C)N)[nH]1] | 287.9 (M + H) |
| 34ⁱ | 5-(1-(2-Amino-2-methylpropyl)-3- (trifluoromethyl)-1H-pyrazol-5- yl)-1,3,4-thiadiazol-2-ol hydrochloride | 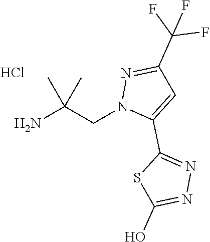 [SMILES: Cl ; CC(C)(N)Cn1nc(C(F)(F)F)cc1-c1nnc(O)s1] | 307.9 (M + H) |

^{a}36 h reaction time, used 10 eq HCl
^{b}16 h reaction time
^{c}EtOAc as co-solvent, 12 eq HCl;
^{d}EtOH was used as co-solvent, 2.5 h reaction time, used 18 eq HCl was used, giving a ~1:4 mixture of regioisomers favoring the title compound
^{e}EtOAc was used as co-solvent, 5 h reaction time, 10 eq HCl was used
^{f}6 h reaction time, 10 eq HCl was used, the product was triturated with DCM/ether to give a solid
^{g}EtOAc was used as co-solvent, used 12 eq HCl, 43 h reaction time
^{h}Used 10 eq HCl
ⁱUsed 1,4-dioxane as solvent, 16 h reaction time

### Preparation 35

### 3-(1-(2-Amino-2-methylpropyl)-3-(trifluoromethyl)-1H-pyrazol-5-yl)pyridin-2(1H)-one hydrochloride

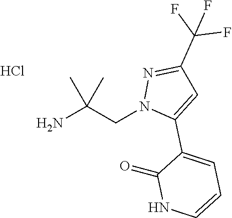 [SMILES: Cl ; CC(C)(N)Cn1nc(C(F)(F)F)cc1-c1ccc[nH]c1=O]

A solution of tert-butyl (1-(5-(2-methoxypyridin-3-yl)-3-(trifluoromethyl)-1H-pyrazol-1-yl)-2-methylpropan-2-yl)carbamate (132.2 mg, 239.2 μmol, ˜75% purity) in DCM (957 μL) was treated with 4M HCl in 1,4-dioxane (179 μL, 718 μmol). After stirring at RT overnight, another portion of 4M HCl in 1,4-dioxane (897 μL, 3.59 mmol) was added, and the resulting mixture was stirred at 50° C. for 3 days. HBr in HOAc (33 wt. % in HOAc, 130.0 μL, 718 μmol) was added, and stirring was continued at 50° C. for 24 h. The reaction mixture was concentrated under a stream of N₂ to obtain a quantitative yield of the title compound (143.1 mg, ˜55% purity) as a brown solid. ES-MS m/z 301.2 (M+H).

### Preparation 36

### (6-(1-(2-Amino-2-methylpropyl)-3-(trifluoromethyl)-1H-pyrazol-5-yl)pyridin-2(1H)-one hydrochloride

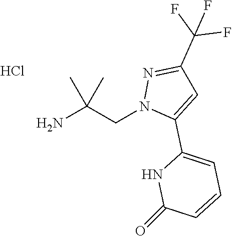 [SMILES: Cl ; CC(C)(N)Cn1nc(C(F)(F)F)cc1-c1cccc(=O)[nH]1]

The title compound was prepared in a manner similar as described in Preparation 35 using tert-butyl (1-(5-(6-methoxypyridin-2-yl)-3-(trifluoromethyl)-1H-pyrazol-1-yl)-2-methylpropan-2-yl)carbamate. ES-MS m/z 301.2 (M+H).

### Preparation 37

### 1-((5-Fluoropyridin-2-yl)methyl)-2,4,6-triphenylpyridin-1-ium tetrafluoroborate

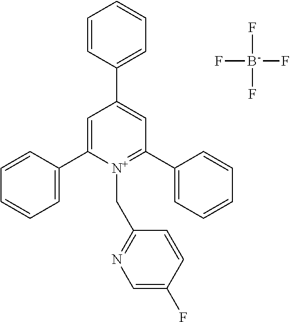 [SMILES: Fc1ccc(C[n+]2c(-c3ccccc3)cc(-c3ccccc3)cc2-c2ccccc2)nc1 ; F[B-](F)(F)F]

(5-Fluoropyridin-2-yl) methanamine (4.99 g, 39.5 mmol) was added to a mixture of 2,4,6-triphenylpyrylium tetrafluoroborate (14.5 g, 36.6 mmol) in DCM (100 mL) at RT, and the resulting mixture was stirred overnight before concentrating to a foam. The foam was triturated with Et₂O, and the solvent was decanted off (repeated). The remaining residue was dried overnight under reduced pressure to obtain the title compound as a yellow foam, which was used directly in the next step assuming quantitative yield. ES-MS m/z 417.0 (M⁺).

### Preparation 38

### 5-Fluoro-2-(2-methyl-2-nitropropyl)pyridine

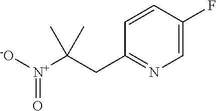

2-Nitropropane (12.9 mL, 144 mmol) was added over 10 min to an ice-water bath cooled solution of sodium methoxide (5.4 molar solution in MeOH, 22.2 mL, 120 mmol) in MeOH (240 mL). The resulting mixture was stirred for 30 min at RT and then concentrated to give a white solid that was dried under reduced pressure overnight. To the white solid was added 1-((5-fluoropyridin-2-yl)methyl)-2,4,6-triphenylpyridin-1-ium tetrafluoroborate (19 g, 38 mmol) and DMSO (50 mL) in a 250 mL, three-neck flask equipped with a mechanical stirrer under N₂. The resulting mixture was stirred at 70° C. for 2 h. The mixture was cooled to RT and then diluted with Et₂O (200 mL) to give a precipitate. The solids were removed by filtration through diatomaceous earth, rinsing with Et₂O. The filtrate (800 mL) was washed with water (200 mL). The layers were separated, and the aq. layer was extracted with ether (3×50 mL). The organic layers were combined, washed with sat. aq. NaCl, dried over MgSO₄, filtered and concentrated. The residue was purified by silica gel chromatography using a gradient of 5-100% EtOAc in DCM to obtain the title compound (5.63 g, 75%) as a yellow oil. ES-MS m/z 198.8 (M+H).

### Preparation 39

### 1-(5-Fluoropyridin-2-yl)-2-methylpropan-2-amine hydrochloride

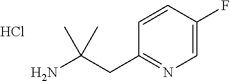

A solution of 5-fluoro-2-(2-methyl-2-nitropropyl)pyridine (4.62 g, 23.3 mmol) in MeOH (100 mL) was added to a mixture of Raney® Ni (19.28 g, 328.5 mmol) in MeOH (100 mL) in a pressure vessel under a stream of N₂. The vessel was sealed, purged with N₂ 5 times, purged with H₂ 5 times, and pressurized with H₂ to 0.41 MPa. After shaking at RT for 5 h, the suspension was filtered over diatomaceous earth, and the solids were rinsed with MeOH to give a clear, light-yellow filtrate, which was combined with the filtrate from a second reaction ran on 996 mg of 5-fluoro-2-(2-methyl-2-nitropropyl)pyridine. The combined filtrate was concentrated. The residue was dissolved in ether (200 mL) and filtered through fluted filter paper to remove solids. The filtrate was concentrated to give 1-(5-fluoropyridin-2-yl)-2-methylpropan-2-amine (4.19 g) as a light orange oil. ES/MS m/z 169.0 (M+H). The oil was dissolved in ether (200 mL) and treated with 4M HCl in 1,4-dioxane (7.0 mL, 28 mmol) to give a precipitate. The solvent was removed under reduced pressure to obtain a quantitative yield of the title compound (6.55 g) as a fluffy, yellow, hygroscopic solid. ES-MS m/z 268.8 (M+H).

### Preparation 40

### Methyl (Z)-2-(((benzyloxy)carbonyl)amino)-3-(1-methyl-1H-pyrazol-4-yl)acrylate

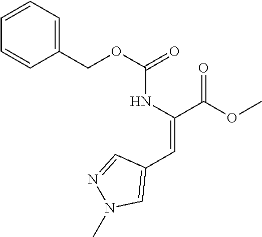 [SMILES: COC(=O)/C(=C/c1cnn(C)c1)NC(=O)OCc1ccccc1]

To a cooled mixture of 1-methyl-1H-pyrazole-4-carbaldehyde (300 g, 2.72 mol) and methyl 2-(((benzyloxy)carbonyl)amino)-2-(dimethoxyphosphoryl)acetate (966 g, 2.92 mol) in DCM (1.5 L) was added DBU (435 mL, 2.89 mol) in one portion at 0-5° C. under N₂. Warmed to 15-20° C. and stirred for 2 h. The reaction mixture was diluted with water (2 L) and extracted with DCM (2×0.5 L). The organic layers were combined, dried over Na₂SO₄, filtered, and concentrated under reduced pressure. The residue was triturated by stirring with MTBE (3 L) for 1 h, before filtering to collect the solids. The reaction was repeated ten times. Combination of the collected material from all eleven reactions gave the title compound (7.00 kg, 74%) as a yellow solid. ES-MS m/z 316.1 (M+H).

The compounds in the following table were prepared as described in Preparation 40 using the appropriate phosphonate and aldehyde. Various methods can be used to purify the compounds, which would be apparent to one skilled in the art.

TABLE 4

| | | | ES-MS |
|---|---|---|---|
| Preparation | Chemical Name | Structure | m/z |
| 41ª | Methyl (Z)-2-((tert- butoxycarbonyl)amino)-3- (imidazo[1,2-a]pyrimidin-3- yl)acrylate | 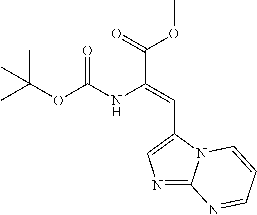 [SMILES: COC(=O)/C(=C/c1cnc2ncccn12)NC(=O)OC(C)(C)C] | 319.1 (M + H) |

^{a}The product was purified by trituration with ACN

### Preparation 42

### Methyl 2-(((benzyloxy)carbonyl)amino)-3-(1-methyl-1H-pyrazol-4-yl)propanoate, Enantiomerically Enriched with the (R)-Enantiomer (˜3:1)

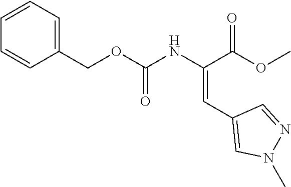 [SMILES: COC(=O)/C(=C\c1cnn(C)c1)NC(=O)OCc1ccccc1]

To a solution of methyl (Z)-2-(((benzyloxy)carbonyl)amino)-3-(1-methyl-1H-pyrazol-4-yl)acrylate (700 g, 2.22 mol) in MeOH (7 L) was added diacetato[(R)-(+)-2,2′-bis(diphenylphosphino)-1,1′-binaphthyl]ruthenium(II) (Ru(OAc)₂[(R)-binap], CAS #325146-81-4), 46.8 g, 55.5 mmol) and HOAc (127 mL, 2.22 mol) under Ar. The resulting mixture was degassed under reduced pressure and purged with H₂ several times. After stirring under H₂ (3.00 MPa) at 60° C. for 16 h, the reaction mixture was concentrated under reduced pressure. The reaction was repeated nine times on the same scale. The residues from all ten reactions were combined to obtain the title compound (6.80 kg, 97%) as a yellow oil. ES-MS m/z 318.1 (M+H).

### Preparation 43

### (R)-2-(((Benzyloxy)carbonyl)amino)-3-(1-methyl-1H-pyrazol-4-yl)propanoic acid

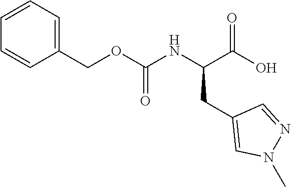 [SMILES: Cn1cc(C[C@@H](NC(=O)OCc2ccccc2)C(=O)O)cn1]

A 0° C. solution of methyl 2-(((benzyloxy)carbonyl)amino)-3-(1-methyl-1H-pyrazol-4-yl)propanoate, enantiomerically enriched with the (R)-enantiomer (3.40 kg, 10.7 mol) in MeOH (10.2 L) was treated with a mixture of LiOH. H₂O (1.80 kg, 42.9 mol) and H₂O (13.6 L). After stirring at 20° C. for 16 h, the mixture was partially concentrated under reduced pressure, adjusted to pH=3-4 with 1 M aq. H₂SO₄, and filtered to collect the solids. The reaction was repeated on the same scale, and the resultant solids were combined and dried under reduced pressure at 45° C. to give 2-(((benzyloxy)carbonyl)amino)-3-(1-methyl-1H-pyrazol-4-yl)propanoic acid, enantiomerically enriched with the (R)-enantiomer (5.00 kg, 77% yield) as a black solid. ES-MS m/z 304.1 (M+H). The material was divided into five equal portions. Each portion (1.00 kg, 3.30 mol) was dissolved in acetone (20 L) and stirred for 10 min at 25° C., before adding (1S,2R)-2-amino-1,2-diphenylethan-1-ol (633 g, 2.97 mol). After stirring at 25° C. for 12 h, the mixtures were filtered, and the isolated solids from the five reactions were combined to give the (1S,2R)-2-amino-1,2-diphenylethan-1-ol salt of the title compound (5.40 kg, 63%) as a white solid. A 0° C. solution of the salt (1.01 kg, 1.96 mol) in water (15 L) was adjusted to pH 9 with 20% aq. Na₂CO₃ and extracted with EtOAc (3×4 L) to remove neutral impurities. The aq. phase was cooled to 0° C., adjusted to pH 2 with H₂SO₄, and extracted with EtOAc (3×4 L). The organic layers were combined, dried over Na₂SO₄, filtered, and concentrated under reduced pressure. The residue was triturated by mixing with EtOAc (10.0 L) at 20° C. for 2 h before filtering to collect the solids. This reaction was repeated five times on the same scale, and the isolated material from all six reactions was combined to give the title compound (2.00 kg, 56% yield) as a white solid. ES-MS m/z 304.1 (M+H). [a]_{D}²⁰=−4.98° (C=1.0, ACN:H₂O, 5:1). >98% ee, Rt=3.44 min (SFC, Chiralpak® AD-3, 150× 4.6 mm, 3 μm, 35° C., gradient 10-50% IPA (with 0.1% IPAm) in CO₂ over 3.5 min, 2.5 mL/min, 220 nm).

### Preparation 44

### Methyl (R)-2-((tert-butoxycarbonyl)amino)-3-(imidazo[1,2-a]pyrimidin-3-yl)propanoate

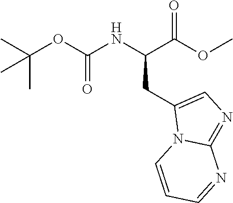 [SMILES: COC(=O)[C@@H](Cc1cnc2ncccn12)NC(=O)OC(C)(C)C]

In a glove box under N₂, methyl (Z)-2-((tert-butoxycarbonyl)amino)-3-(imidazo[1,2-a]pyrimidin-3-yl)acrylate (20.28 g, 63.71 mmol) and Rh-COD-[(R)-MaxPhos]-BF₄ (CAS #1263077-53-7, 778 mg, 1.39 mmol) were added to a 600 mL autoclave. 1,1,1,3,3,3-hexafluoroisopropanol (33 mL, degassed) and 2,2,2-trifluoroethanol (367 mL, degassed) were added. The autoclave was sealed, purged with H₂, and then pressurized to 1.7 MPa with H₂. The reaction temperature was gradually raised from RT to 60° C. over 30 min while stirring. After heating for 8 h, the reaction mixture was cooled to RT and concentrated under reduced pressure. The orange liquid residue was dissolved in DCM (200 mL) and concentrated under reduced pressure twice, followed by a third time with MTBE (250 mL), to give an orange glass (28.8 g) which was crystallized from MTBE (200 mL). The solids were collected by filtration, rinsing with cold MTBE (100 mL). Drying at 50° C. under reduced pressure provided the title compound (8.13 g, 40%) as a tan powder. ES-MS m/z 321.1 (M+H). [a]_{D}²⁰=+48.8° (C=0.2, MeOH). [a]_{D}²⁰=−10° (c=0.2, CHCl₃). >99% ee, Rt=1.33 min (SFC, ChiralCel® OD-H, 4.6×100 mm, 5 μm, 40° C., 20% IPA: 80% CO₂, 5 mL/min, 225 nm).

### Preparation 45

### Benzyl (R)-4-((1-methyl-1H-pyrazol-4-yl)methyl)-5-oxooxazolidine-3-carboxylate

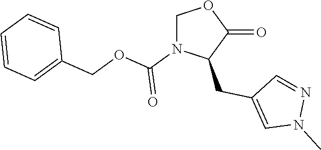 [SMILES: Cn1cc(C[C@@H]2C(=O)OCN2C(=O)OCc2ccccc2)cn1]

A solution of (R)-2-(((benzyloxy)carbonyl)amino)-3-(1-methyl-1H-pyrazol-4-yl)propanoic acid (190 g, 626 mmol) in toluene (7.22 L) was treated with p-toluene sulfonic acid monohydrate (23.8 g, 125 mmol) and paraformaldehyde (73.3 g, 2.44 mol). The resulting mixture was stirred at 120° C. for 16 h before concentrating under reduced pressure. The residue was purified by silica gel chromatography using a gradient of 0-100% EtOAc in PE to obtain the title compound (145 g, 73%) as a colorless oil. ES-MS m/z 316.3 (M+H).

### Preparation 46

### (R)-2-(((benzyloxy)carbonyl)amino)-3-(1-methyl-1H-pyrazol-4-yl)propanoic acid

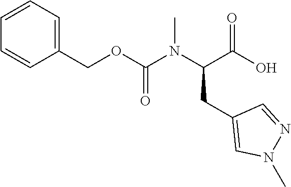 [SMILES: CN(C(=O)OCc1ccccc1)[C@H](Cc1cnn(C)c1)C(=O)O]

To a solution of benzyl (R)-4-((1-methyl-1H-pyrazol-4-yl)methyl)-5-oxooxazolidine-3-carboxylate (145 g, 460 mmol) in DCE (1.45 L) was added triethylsilane (534 g, 4.60 mol), TFA (1.57 kg, 13.80 mol), and 4 M HCl/dioxane (115 mL). The resulting mixture was stirred at 70° C. for 3 h then concentrated under reduced pressure. H₂O (500 mL) was added, followed by 5N aq. NaOH to bring the mixture above pH 10. Extracted with DCM (2×500 mL). The aq. layer was acidified below pH 2 with 5N HCl and extracted with DCM (2×500 mL). This second set of organic layers were combined, dried over Na₂SO₄, filtered, and concentrated under reduced pressure to afford the title compound (100 g, 68.5%) as colorless oil. ES-MS m/z 318.1 (M+H). [a]_{D}²⁰=+38.7° (C=1.0, ACN). 97.5% ee, Rt=2.38 min (SFC, Chiralpak® IG-3, 4.6×100 mm, 3 μm, 35° C., gradient 10-50% EtOH (with 0.2% 7M NH₃ in MeOH) in CO₂ over 3.4 min, 3.4 mL/min, 220 nm).

### Preparation 47

### Methyl (R)-2-((tert-butoxycarbonyl)amino)-3-(1-methyl-1H-pyrazol-4-yl)propanoate

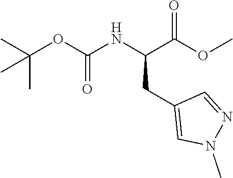 [SMILES: COC(=O)[C@@H](Cc1cnn(C)c1)NC(=O)OC(C)(C)C]

A mixture of 4-iodo-1-methyl-1H-pyrazole (20.0 g, 96.2 mmol), pyridine-2,6-bis(carboximidamide) dihydrochloride (5.05 g, 19.2 mmol), nickel(II) chloride, dimethoxyethane adduct (CAS #29046-78-4, 4.23 g, 19.2 mmol), methyl(S)-2-((tert-butoxycarbonyl)amino)-3-iodopropanoate (47.5 g, 144 mmol) and Zinc (8.46 g, 129 mmol) in DMA (300 mL) was purged with N₂ and stirred at 40° C. for 16 h under N₂. The reaction mixture was quenched slowly with sat. aq. NH₄Cl solution (300 mL) at 0° C. then filtered. The filtrate was diluted with water (200 mL) and extracted with EtOAc (3×400 mL). The organic layers were combined, washed with water (2×300 mL), then sat. aq. NaCl (300 mL), dried over anhydrous Na₂SO₄, filtered, and concentrated under reduced pressure. The residue was purified by silica gel chromatography using a gradient of 0-33% EtOAc in PE to obtain the title compound (12.16 g, 42%) as a colorless oil. ES-MS m/z 284.1 (M+H). >99% ee, Rt=0.55 min (SFC, Chiralcel® OJ-3, 4.6×150 mm, 3 μm, 35° C., gradient 5-50% EtOH (with 0.05% DMEA) in CO₂ over 1.4 min, 3 mL/min, 220 nm).

### Preparation 48

### Methyl (R)-2-((tert-butoxycarbonyl)amino)-3-(1-(difluoromethyl)-1H-pyrazol-4-yl)propanoate

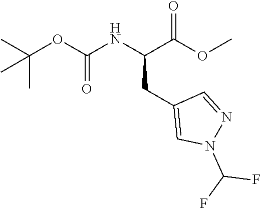 [SMILES: COC(=O)[C@@H](Cc1cnn(C(F)F)c1)NC(=O)OC(C)(C)C]

To a solution of 1-difluoromethyl-4-iodo-1H-pyrazole (15.0 g, 59.6 mmol) in DMA (50 mL) under N₂, was added pyridine-2,6-bis(carboximidamide) dihydrochloride (2.83 g, 12.0 mmol), methyl(S)-2-((tert-butoxycarbonyl)amino)-3-iodopropanoate (29.5 g, 89.7 mmol), Zinc (7.79 g, 119 mmol) and nickel(II) chloride, dimethoxyethane adduct (CAS #29046-78-4, 2.60 g, 11.8 mmol). The resulting mixture was stirred at 60° C. (internally) for 12 h. The reaction mixture was quenched with sat. aq. NH₄Cl solution (200 mL) then filtered over diatomaceous earth, rinsing with EtOAc (2×50 mL). The filtrate was diluted with water (200 mL) and extracted with EtOAc (3×100 mL). The organic layers were combined, washed sat. aq. NaCl (100 mL), dried over Na₂SO₄, filtered, and concentrated under reduced pressure. The residue was purified by silica gel chromatography using a gradient of 0-10% EtOAc in PE to obtain the title compound (13.0 g, 67%) as a colorless oil. ES-MS m/z 264.1 (M-tBu+H). >99% ee, Rt=1.38 min (SFC, Chiralpak® AD-3, 4.6×150 mm, 3 μm, 35° C., gradient 10-50% EtOH (with 0.2% 7M NH₃ in MeOH) in CO₂ over 3 min, 2.5 mL/min, 220 nm).

### Preparation 50

### (R)-2-((tert-Butoxycarbonyl)amino)-3-(1-(difluoromethyl)-1H-pyrazol-4-yl)propanoic acid

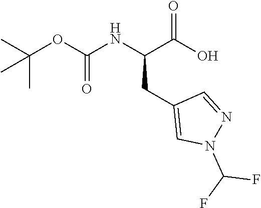 [SMILES: CC(C)(C)OC(=O)N[C@H](Cc1cnn(C(F)F)c1)C(=O)O]

A solution of methyl (R)-2-((tert-butoxycarbonyl)amino)-3-(1-(difluoromethyl)-1H-pyrazol-4-yl)propanoate (12.0 g, 36.8 mmol) in THF (120 mL) was treated with 1M aq. LiOH. H₂O (110 mL, 110 mmol) and stirred at 25° C. for 1 h under N₂. The mixture was extracted with DCM (3×30 mL). The aq. phase was adjusted to pH 2-3 with 1N aq. HCl and the resulting white solid was collected by filtration, suspended in PE (100 mL) and stirred for 12 h. Filtration and drying under reduced pressure afforded the title compound (10.5 g, 94%) as a white solid. ES-MS m/z 250.1 (M-tBu+H). >99% ee, Rt=2.73 min (SFC, Chiralpak® AD-3, 4.6×150 mm, 3 μm, 35° C., 15% IPA (with 0.5% IPAm): 85% CO₂, 2.5 mL/min, 220 nm).

The compounds in the following table were prepared as described in Preparation 50. Alternatively, after acidification of the reaction mixture, the products can be extracted with EtOAc. These compounds could also be isolated as their corresponding Lithium carboxylate salts by concentrating the basic reaction mixture, which would be apparent to one skilled in the art.

TABLE 5

| Prep | Chemical Name | Structure | ES-MS m/z |
|---|---|---|---|
| 51 | (R)-2-((tert- Butoxycarbonyl)amino)- 3-(1-methyl-1H- pyrazol-4-yl)propanoic acid | 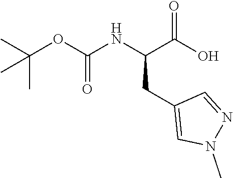 [SMILES: Cn1cc(C[C@@H](NC(=O)OC(C)(C)C)C(=O)O)cn1] | 270.2 (M + H) |
| 52 | (R)-2-((tert- Butoxycarbonyl)amino)- 3-(imidazo[1,2- a]pyrimidin-3- yl)propanoic acid | 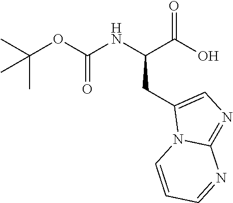 [SMILES: CC(C)(C)OC(=O)N[C@H](Cc1cnc2ncccn12)C(=O)O] | 307.0 (M + H) |

### Preparation 53

### tert-Butyl (R)-(1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate

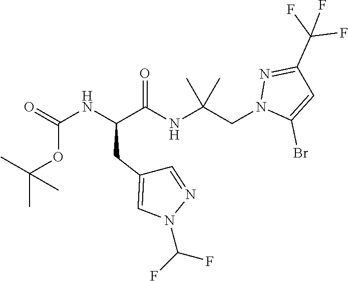 [SMILES: CC(C)(Cn1nc(C(F)(F)F)cc1Br)NC(=O)[C@@H](Cc1cnn(C(F)F)c1)NC(=O)OC(C)(C)C]

A stirring mixture of (R)-2-((tert-butoxycarbonyl)amino)-3-(1-(difluoromethyl)-1H-pyrazol-4-yl)propanoic acid (8.3 g, 27 mmol), 1-(5-bromo-3-(trifluoromethyl)-1H-pyrazol-1-yl)-2-methylpropan-2-amine hydrochloride (8.4 g, 26 mmol), DIEA (16 mL, 91 mmol), and DMF (50 mL) was cooled at 0° C. and treated with HATU (12 g, 31 mmol) portion-wise. After 4 h at RT, the reaction mixture was partitioned between EtOAc (400 mL) and water (400 mL). The aq. layer was extracted with EtOAc (300 mL). The organic layers were combined, washed with water (500 mL), then sat. aq. NaCl (500 mL), dried over Na₂SO₄, filtered, and concentrated under reduced pressure. The residue was purified by silica gel chromatography using a gradient of 50-65% MTBE in heptane to obtain the title compound (14.4 g, 96%) as a white foam. ES-MS m/z 517,519 (M-tBu+H).

The compounds in the following table were prepared as described in Preparation 53 using the appropriate carboxylic acid and the appropriate amine. Reactants can be added in different orders or in differing equivalency. DMA and DMSO are suitable replacements for DMF. Reaction times (30 min to overnight) and temperatures (0° C. to RT) may vary, and differing methods can be used to work up or purify the compounds (normal phase, or high, neutral or low pH reversed phase). Such variances would be apparent to one skilled in the art.

TABLE 6

| | | | ES-MS |
|---|---|---|---|
| Prep | Chemical Name | Structure | m/z |
| 54 | tert-Butyl (R)-(1-((1-(5-bromo-3- (trifluoromethyl)-1H-pyrazol-1- yl)-2-methylpropan-2-yl)amino)- 3-(1-methyl-1H-pyrazol-4-yl)-1- oxopropan-2-yl)carbamate | 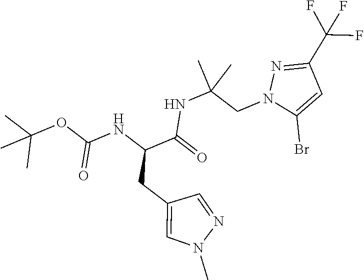 [SMILES: Cn1cc(C[C@@H](NC(=O)OC(C)(C)C)C(=O)NC(C)(C)Cn2nc(C(F)(F)F)cc2Br)cn1] | 481, 483 (M − tBu + H) |
| 55 | Benzyl (R)-(1-((1-(5-bromo-3- (trifluoromethyl)-1H-pyrazol-1- yl)-2-methylpropan-2-yl)amino)- 3-(1-methyl-1H-pyrazol-4-yl)-1- oxopropan-2-yl)carbamate | 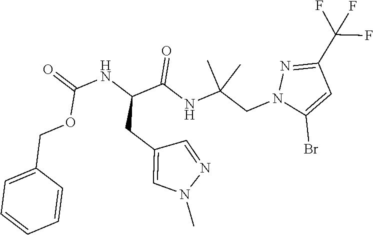 [SMILES: Cn1cc(C[C@@H](NC(=O)OCc2ccccc2)C(=O)NC(C)(C)Cn2nc(C(F)(F)F)cc2Br)cn1] | 571, 573 (M − tBu + H) |
| 56 | Benzyl (R)-(1-((2-methyl-1-(4- (trifluoromethyl)-1H-pyrazol-1- yl)propan-2-yl)amino)-3-(1- methyl-1H-pyrazol-4-yl)-1- oxopropan-2-yl)carbamate | 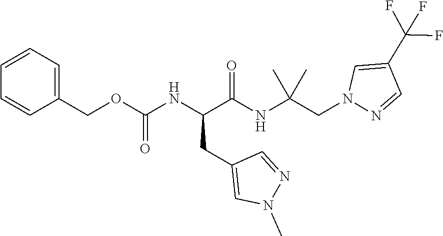 [SMILES: Cn1cc(C[C@@H](NC(=O)OCc2ccccc2)C(=O)NC(C)(C)Cn2cc(C(F)(F)F)cn2)cn1] | 493 (M + H) |
| 57 | tert-Butyl (R)-(1-((2-methyl-1- (4-(trifluoromethyl)-1H-pyrazol- 1-yl)propan-2-yl)amino)-3-(1- methyl-1H-pyrazol-4-yl)-1- oxopropan-2-yl)carbamate | 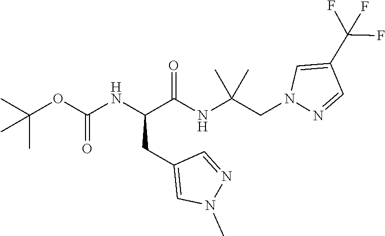 [SMILES: Cn1cc(C[C@@H](NC(=O)OC(C)(C)C)C(=O)NC(C)(C)Cn2cc(C(F)(F)F)cn2)cn1] | 459 (M + H) |
| 58 | Benzyl (R)-(1-((2-methyl-1-(5- (1-methyl-6-oxo-1,6- dihydropyridin-3-yl)-3- (trifluoromethyl)-1H-pyrazol-1- yl)propan-2-yl)amino)-3-(1- methyl-1H-pyrazol-4-yl)-1- oxopropan-2-yl)carbamate | 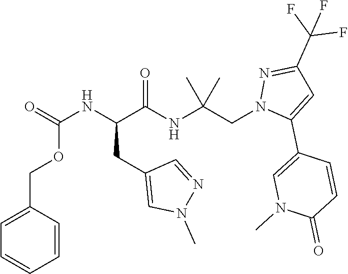 [SMILES: Cn1cc(C[C@@H](NC(=O)OCc2ccccc2)C(=O)NC(C)(C)Cn2nc(C(F)(F)F)cc2-c2ccc(=O)n(C)c2)cn1] | 600 (M + H) |
| 59 | tert-Butyl (R)-(1-((2-methyl-1- (5-(1-methyl-6-oxo-1,6- dihydropyridin-3-yl)-3- (trifluoromethyl)-1H-pyrazol-1- yl)propan-2-yl)amino)-3-(1- methyl-1H-pyrazol-4-yl)-1- oxopropan-2-yl)carbamate | 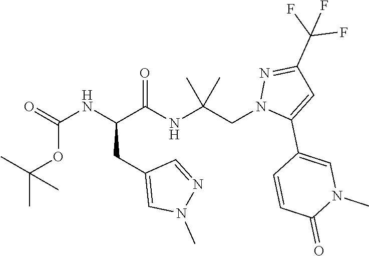 [SMILES: Cn1cc(C[C@@H](NC(=O)OC(C)(C)C)C(=O)NC(C)(C)Cn2nc(C(F)(F)F)cc2-c2ccc(=O)n(C)c2)cn1] | 566 (M + H) |
| 60 | tert-Butyl (R)-(1-((2-methyl-1- (5-(2-oxopyridin-1(2H)-yl)-3- (trifluoromethyl)-1H-pyrazol-1- yl)propan-2-yl)amino)-3-(1- methyl-1H-pyrazol-4-yl)-1- oxopropan-2-yl)carbamate | 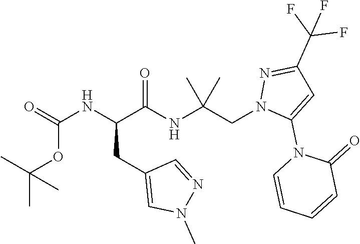 [SMILES: Cn1cc(C[C@@H](NC(=O)OC(C)(C)C)C(=O)NC(C)(C)Cn2nc(C(F)(F)F)cc2-n2ccccc2=O)cn1] | 552 (M + H) |
| 61 | tert-Butyl (R)-(1-((1-(5-(1H- 1,2,3-triazol-1-yl)-3- (trifluoromethyl)-1H-pyrazol-1- yl)-2-methylpropan-2-yl)amino)- 3-(1-methyl-1H-pyrazol-4-yl)-1- oxopropan-2-yl)carbamate | 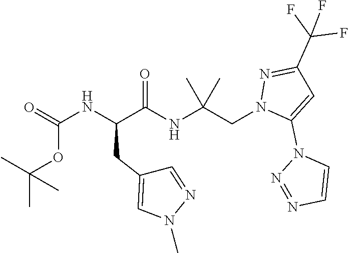 [SMILES: Cn1cc(C[C@@H](NC(=O)OC(C)(C)C)C(=O)NC(C)(C)Cn2nc(C(F)(F)F)cc2-n2ccnn2)cn1] | 526 (M + H) |
| 62 | tert-Butyl (R)-(1-((1-(5-(2H- 1,2,3-triazol-2-yl)-3- (trifluoromethyl)-1H-pyrazol-1- yl)-2-methylpropan-2-yl)amino)- 3-(1-methyl-1H-pyrazol-4-yl)-1- oxopropan-2-yl)carbamate | 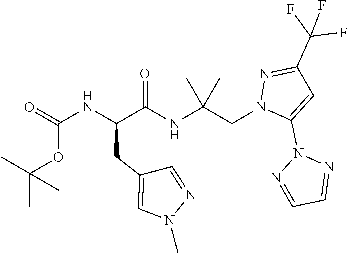 [SMILES: Cn1cc(C[C@@H](NC(=O)OC(C)(C)C)C(=O)NC(C)(C)Cn2nc(C(F)(F)F)cc2-n2nccn2)cn1] | 526 (M + H) |
| 63 | Methyl (R)-1-(2-(2-((tert- butoxycarbonyl)amino)-3-(1- methyl-1H-pyrazol-4- yl)propanamido)-2- methylpropyl)-3- (trifluoromethyl)-1H-pyrazole-5- carboxylate | 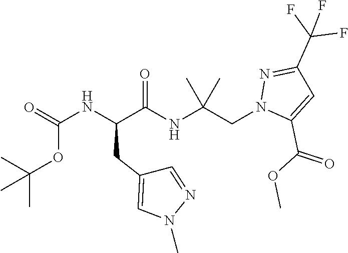 [SMILES: COC(=O)c1cc(C(F)(F)F)nn1CC(C)(C)NC(=O)[C@@H](Cc1cnn(C)c1)NC(=O)OC(C)(C)C] | 517 (M + H) |
| 64 | tert-Butyl (R)-(1-((1-(5-(4H- 1,2,4-triazol-3-yl)-3- (trifluoromethyl)-1H-pyrazol-1- yl)-2-methylpropan-2-yl)amino)- 3-(1-methyl-1H-pyrazol-4-yl)-1- oxopropan-2-yl)carbamate | 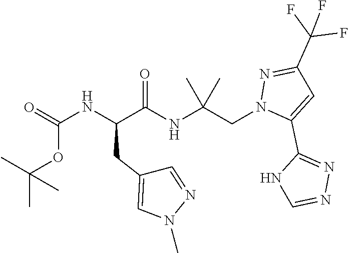 [SMILES: Cn1cc(C[C@@H](NC(=O)OC(C)(C)C)C(=O)NC(C)(C)Cn2nc(C(F)(F)F)cc2-c2nnc[nH]2)cn1] | 526 (M + H) |
| 65 | tert-Butyl (R)-(1-((2-methyl-1- (5-(2-methyl-1H-imidazol-5-yl)- 3-(trifluoromethyl)-1H-pyrazol- 1-yl)propan-2-yl)amino)-3-(1- methyl-1H-pyrazol-4-yl)-1- oxopropan-2-yl)carbamate | 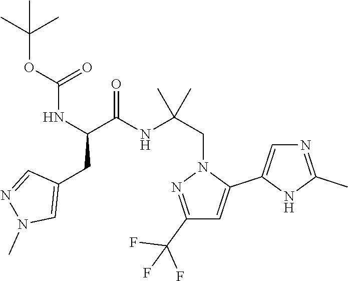 [SMILES: Cc1ncc(-c2cc(C(F)(F)F)nn2CC(C)(C)NC(=O)[C@@H](Cc2cnn(C)c2)NC(=O)OC(C)(C)C)[nH]1] | 539 (M + H) |
| 66 | tert-Butyl (R)-(1-((2-methyl-1- (5-(5-oxo-4,5-dihydro-1,3,4- thiadiazol-2-yl)-3- (trifluoromethyl)-1H-pyrazol-1- yl)propan-2-yl)amino)-3-(1- methyl-1H-pyrazol-4-yl)-1- oxopropan-2-yl)carbamate | 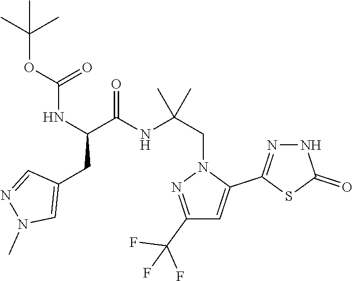 [SMILES: Cn1cc(C[C@@H](NC(=O)OC(C)(C)C)C(=O)NC(C)(C)Cn2nc(C(F)(F)F)cc2-c2n[nH]c(=O)s2)cn1] | 559 (M + H) |
| 67 | Benzyl (R)-(1-((1-(5- cyclopropyl-3-(trifluoromethyl)- 1H-pyrazol-1-yl)-2- methylpropan-2-yl)amino)-3-(1- methyl-1H-pyrazol-4-yl)-1- oxopropan-2-yl)carbamate | 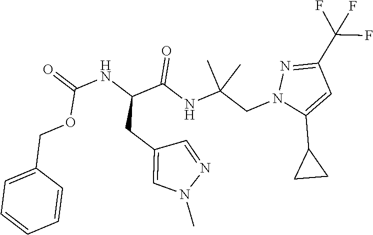 [SMILES: Cn1cc(C[C@@H](NC(=O)OCc2ccccc2)C(=O)NC(C)(C)Cn2nc(C(F)(F)F)cc2C2CC2)cn1] | 533 (M + H) |
| 68 | tert-Butyl (R)-(1-((1-(5-bromo-3- (trifluoromethyl)-1H-pyrazol-1- yl)-2-methylpropan-2-yl)amino)- 3-(imidazo[1,2-a]pyrimidin-3- yl)-1-oxopropan-2-yl)carbamate | 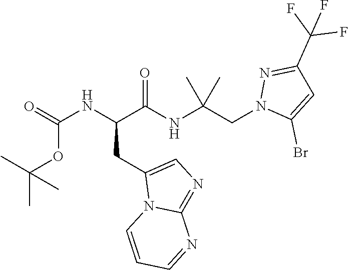 [SMILES: CC(C)(Cn1nc(C(F)(F)F)cc1Br)NC(=O)[C@@H](Cc1cnc2ncccn12)NC(=O)OC(C)(C)C] | 574, 576 (M + H) |
| 69 | tert-Butyl (R)-(3-(imidazo[1,2- a]pyrimidin-3-y1)-1-((2-methyl- 1-(4-(trifluoromethyl)-1H- pyrazol-1-yl)propan-2-yl)amino)- 1-oxopropan-2-yl)carbamate | 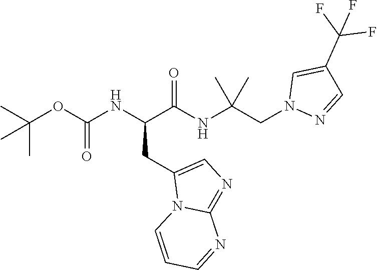 [SMILES: CC(C)(Cn1cc(C(F)(F)F)cn1)NC(=O)[C@@H](Cc1cnc2ncccn12)NC(=O)OC(C)(C)C] | 496.2 (M + H) |
| 70 | tert-Butyl (R)-(3-(imidazo[1,2- a]pyrimidin-3-yl)-1-((2-methyl- 1-(3-(trifluoromethyl)-1H- pyrazol-1-yl)propan-2-yl)amino)- 1-oxopropan-2-yl)carbamate | 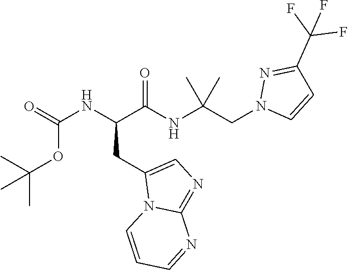 [SMILES: CC(C)(Cn1ccc(C(F)(F)F)n1)NC(=O)[C@@H](Cc1cnc2ncccn12)NC(=O)OC(C)(C)C] | 496.2 (M + H) |
| 71 | tert-Butyl (R)-(1-((1-(5- cyclopropyl-3-(trifluoromethyl)- 1H-pyrazol-1-yl)-2- methylpropan-2-yl)amino)-3- (imidazo[1,2-a]pyrimidin-3-yl)- 1-oxopropan-2-yl)carbamate | 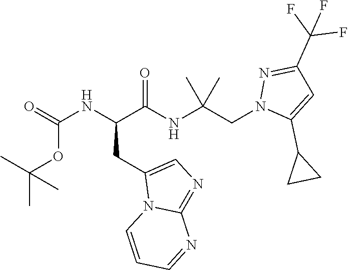 [SMILES: CC(C)(Cn1nc(C(F)(F)F)cc1C1CC1)NC(=O)[C@@H](Cc1cnc2ncccn12)NC(=O)OC(C)(C)C] | 536.2 (M + H) |
| 72 | Benzyl (R)-(1-((1-(5-bromo-3- (trifluoromethyl)-1H-pyrazol-1- yl)-2-methylpropan-2-yl)amino)- 3-(1-methyl-1H-pyrazol-4-yl)-1- oxopropan-2- yl)(methyl)carbamate | 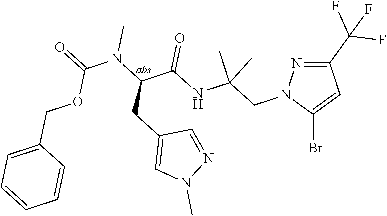 [SMILES: CN(C(=O)OCc1ccccc1)[C@H](Cc1cnn(C)c1)C(=O)NC(C)(C)Cn1nc(C(F)(F)F)cc1Br] | 585, 587 (M + H) |
| 73 | Benzyl (R)-methyl(1-((2-methyl- 1-(5-(5-oxo-4,5-dihydro-1,3,4- thiadiazol-2-yl)-3- (trifluoromethyl)-1H-pyrazol-1- yl)propan-2-yl)amino)-3-(1- methyl-1H-pyrazol-4-yl)-1- oxopropan-2-yl)carbamate | 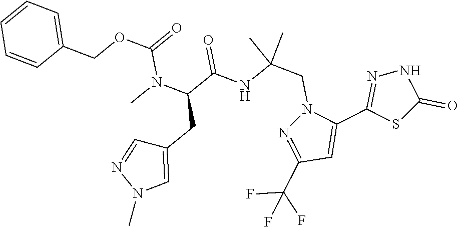 [SMILES: CN(C(=O)OCc1ccccc1)[C@H](Cc1cnn(C)c1)C(=O)NC(C)(C)Cn1nc(C(F)(F)F)cc1-c1n[nH]c(=O)s1] | 607.1 (M + H) |
| 74 | Benzyl (R)-methyl(1-((2-methyl- 1-(5-(2-oxo-1,2-dihydropyridin- 3-yl)-3-(trifluoromethyl)-1H- pyrazol-1-yl)propan-2-yl)amino)- 3-(1-methyl-1H-pyrazol-4-yl)-1- oxopropan-2-yl)carbamate | 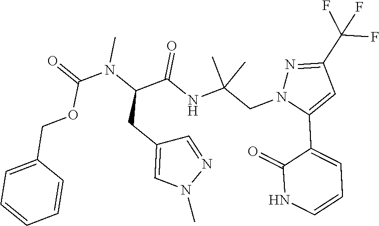 [SMILES: CN(C(=O)OCc1ccccc1)[C@H](Cc1cnn(C)c1)C(=O)NC(C)(C)Cn1nc(C(F)(F)F)cc1-c1ccc[nH]c1=O] | 600.2 (M + H) |
| 75 | Benzyl (R)-methyl(1-((2-methyl- 1-(5-(6-oxo-1,6-dihydropyridin- 2-yl)-3-(trifluoromethyl)-1H- pyrazol-1-yl)propan-2-yl)amino)- 3-(1-methyl-1H-pyrazol-4-yl)-1- oxopropan-2-yl)carbamate | 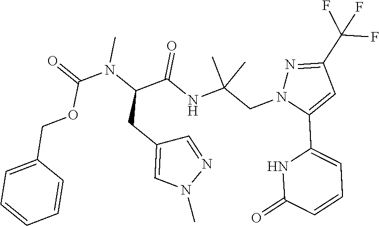 [SMILES: CN(C(=O)OCc1ccccc1)[C@H](Cc1cnn(C)c1)C(=O)NC(C)(C)Cn1nc(C(F)(F)F)cc1-c1cccc(=O)[nH]1] | 600.4 (M + H) |
| 76 | Benzyl (R)-(1-((1-(4- fluorophenyl)-2-methylpropan-2- yl)amino)-3-(1-methyl-1H- pyrazol-4-yl)-1-oxopropan-2- yl)carbamate | 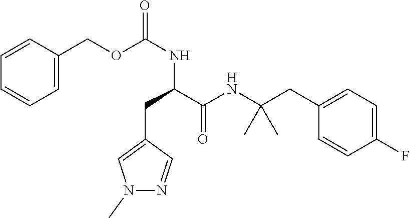 [SMILES: Cn1cc(C[C@@H](NC(=O)OCc2ccccc2)C(=O)NC(C)(C)Cc2ccc(F)cc2)cn1] | 453.3 (M + H) |
| 77 | tert-Butyl (R)-(1-((1-(4- fluorophenyl)-2-methylpropan-2- yl)amino)-3-(imidazo[1,2- a]pyrimidin-3-yl)-1-oxopropan- 2-yl)carbamate | 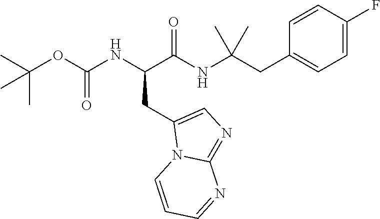 [SMILES: CC(C)(Cc1ccc(F)cc1)NC(=O)[C@@H](Cc1cnc2ncccn12)NC(=O)OC(C)(C)C] | 456.1 (M + H) |
| 78 | tert-Butyl (R)-(1-((1-(5- fluoropyridin-2-yl)-2- methylpropan-2-yl)amino)-3- (imidazo[1,2-a]pyrimidin-3-yl)- 1-oxopropan-2-yl)carbamate | 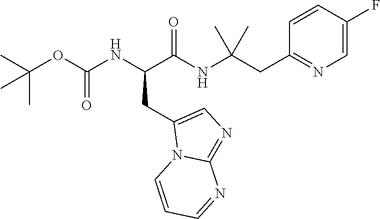 [SMILES: CC(C)(Cc1ccc(F)cn1)NC(=O)[C@@H](Cc1cnc2ncccn12)NC(=O)OC(C)(C)C] | 457.2 (M + H) |

### Preparation 79

### tert-Butyl (R)-(1-((1-(5-(2H-1,2,3-triazol-4-yl)-3-(trifluoromethyl)-1H-pyrazol-1-yl)-2-methylpropan-2-yl)amino)-3-(1-methyl-1H-pyrazol-4-yl)-1-oxopropan-2-yl)carbamate

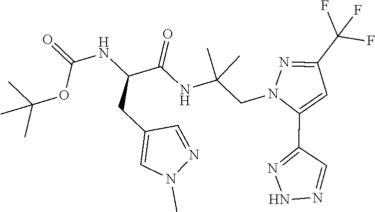 [SMILES: Cn1cc(C[C@@H](NC(=O)OC(C)(C)C)C(=O)NC(C)(C)Cn2nc(C(F)(F)F)cc2-c2cn[nH]n2)cn1]

A solution of tert-butyl (1-(5-(2-(methoxymethyl)-2H-1,2,3-triazol-4-yl)-3-(trifluoromethyl)-1H-pyrazol-1-yl)-2-methylpropan-2-yl)carbamate (914 mg, 2.18 mmol) in EtOAc (6.2 mL) was treated with 4M HCl in 1,4-dioxane (6.6 mL, 26.2 mmol). The resulting mixture was stirred at RT for 17 h, then at 45° C. for 5 h, followed by RT for 3 days. TFA (2.0 mL, 26.2 mmol) was added, and heating was resumed at 45° C. for 19 h. The mixture was concentrated under a stream of N₂. The residue was dissolved in TFA (6.2 mL, 54.7 mmol), and heating was resumed at 45° C. for 1.25 h before concentrating under a stream of N₂ to obtain a quantitative yield of 1-(5-(2H-1,2,3-triazol-4-yl)-3-(trifluoromethyl)-1H-pyrazol-1-yl)-2-methylpropan-2-amine trifluoroacetate. ES-MS m/z 275.2 (M+H). The residue and (R)-2-((tert-butoxycarbonyl)amino)-3-(1-methyl-1H-pyrazol-4-yl)propanoic acid (588 mg, 2.19 mmol) were combined in DMF (5.5 mL) and treated with HATU (790 mg, 3.28 mmol) and DIEA (381 μL, 2.19 mmol). After stirring at RT overnight, DIEA (381 μL, 2.19 mmol) and HATU (790 mg, 3.28 mmol) were added, and stirring was continued for 2.25 h. The reaction mixture was partitioned between sat. aq. NaHCO₃ (15 mL) and 1:1 EtOAc:toluene (20 mL), and the aq. layer was extracted with EtOAc (3×20 mL). The organic layers were combined, washed with sat. aq. NaCl (15 mL), dried over Na₂SO₄, filtered, and concentrated under reduced pressure. The residue was purified by silica gel chromatography using a gradient of 0-20% (7M NH₃/MeOH) in DCM to obtain the title compound (282 mg, 25%) as a yellow liquid. ES-MS m/z 526.2 (M+H).

### Preparation 80

### Benzyl ((2R)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-3-(1-methyl-1H-pyrazol-4-yl)-1-oxopropan-2-yl)carbamate

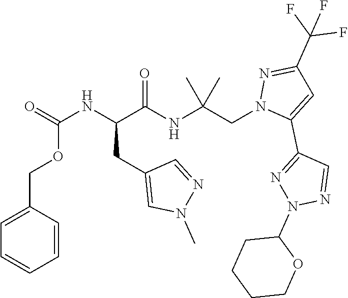 [SMILES: Cn1cc(C[C@@H](NC(=O)OCc2ccccc2)C(=O)NC(C)(C)Cn2nc(C(F)(F)F)cc2-c2cnn(C3CCCCO3)n2)cn1]

A mixture of benzyl (R)-(1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)carbamate (13.4 g, 23.5 mmol) and Na₂CO₃ (7.46 g, 70.4 mmol) in MeOH (50 mL) was sparged with N₂ for 30 min. Bis(di-tert-butyl (4-dimethylaminophenyl)phosphine)dichloropalladium (II), Pd(amphos)Cl₂ (CAS #887919-35-9, 1.66 g, 2.35 mmol) and 2-(tetrahydro-2H-pyran-2-yl)-4-(4,4,5,5-tetramethyl-1,3,2-dioxaborolan-2-yl)-2H-1,2,3-triazole (10.8 g, 38.7 mmol) were added, and degassing was continued for 2 min. The vessel was sealed and stirred at 75° C. overnight. The reaction mixture was partitioned between EtOAc and water. Sat. aq. NaCl was added, and the layers were separated. The aq. layer was extracted twice more with EtOAc. The organic layers were combined, dried over Na₂SO₄, filtered, and concentrated under reduced pressure. The residue was purified by silica gel chromatography using a gradient of 0-30% (3:1 EtOAc:EtOH) in cyclohexane to obtain the title compound (12.8 g, 85%) as a yellow glass. ES-MS m/z 644.4 (M+H).

### Preparation 81

### 2-(4-Bromopyridin-2-yl)propan-2-ol

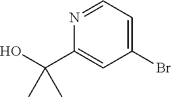

A mixture of ethyl 4-bromopicolinate (2.2 g, 9.6 mmol) in THF (10 mL) was treated with 3M methyl magnesium bromide in Et₂O (9.6 mL, 29 mmol) at 0° C. The reaction mixture was stirred at 0° C. for 2 h, then quenched with water (10 mL) and extracted with EtOAc (2×10 mL). The organic layers were combined, dried over Na₂SO₄, filtered, and concentrated under reduced pressure. The residue was purified by silica gel chromatography using a gradient of 18% EtOAc in PE, to give the title compound (1.6 g, 69%) as a colorless oil. ES-MS m/z 216,218 (M+H).

### Preparation 82

### 4-Bromo-2-(2-((tert-butyldimethylsilyl)oxy)propan-2-yl)pyridine

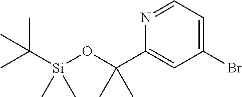 [SMILES: CC(C)(O[Si](C)(C)C(C)(C)C)c1cc(Br)ccn1]

A 0° C. solution of 2-(4-bromopyridin-2-yl)propan-2-ol (1.6 g, 7.0 mmol), TEA (2 mL, 10 mmol) and DMAP (86 mg, 0.70 mmol) in DMF (10 mL) was treated with tert-butyldimethylsilyl chloride (1.5 g, 10 mmol) and the resulting mixture was stirred at 100° C. for 48 h. The reaction mixture was cooled and combined with a second reaction ran in a similar manner on 650 mg of 2-(4-bromopyridin-2-yl)propan-2-ol. The mixture was diluted with water (30 mL) and extracted with EtOAc (2×20 mL). The organic layers were combined, washed with sat. aq. NaCl (2× 30 mL), dried over Na₂SO₄, filtered, and concentrated under reduced pressure. The residue was purified by silica gel chromatography using a gradient of 0-6% EtOAc in PE to obtain the title compound (1.4 g, 42%) as a yellow oil. ES-MS m/z 330,332 (M+H).

### Preparation 83

### 2-(2-((tert-Butyldimethylsilyl)oxy)propan-2-yl)-4-(4,4,5,5-tetramethyl-1,3,2-dioxaborolan-2-yl)pyridine

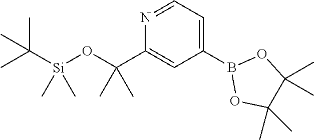 [SMILES: CC(C)(O[Si](C)(C)C(C)(C)C)c1cc(B2OC(C)(C)C(C)(C)O2)ccn1]

To a N₂ sparged solution of 4-bromo-2-(2-((tert-butyldimethylsilyl)oxy)propan-2-yl)pyridine (1.4 g, 4.0 mmol), bis(pinacolato) diborane (2.6 g, 10 mmol) and KOAc (99 mg, 10 mmol) in 1,4-dioxane (10 mL) was added Pd(dppf)Cl₂ (290 mg, 0.40 mmol). The mixture was stirred at 100° C. for 4 h under N₂. The reaction mixture was cooled, and water (20 mL) was added. The mixture was extracted with EtOAc (2×20 mL). The organic layers were combined, washed with sat. aq. NaCl (3×10 mL), dried over Na₂SO₄, filtered, and concentrated under reduced pressure. The residue was purified by silica gel chromatography using a gradient of 0-25% EtOAc in PE to obtain the title compound (1.2 g, 63%, 80% purity). TLC (1:3 EtOAc:PE) R_{f}=0.6.

### Preparation 84

### tert-Butyl (R)-(1-((1-(5-(2-(2-((tert-butyldimethylsilyl)oxy)propan-2-yl)pyridin-4-yl)-3-(trifluoromethyl)-1H-pyrazol-1-yl)-2-methylpropan-2-yl)amino)-3-(1-methyl-1H-pyrazol-4-yl)-1-oxopropan-2-yl)carbamate

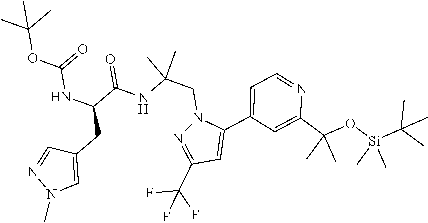 [SMILES: Cn1cc(C[C@@H](NC(=O)OC(C)(C)C)C(=O)NC(C)(C)Cn2nc(C(F)(F)F)cc2-c2ccnc(C(C)(C)O[Si](C)(C)C(C)(C)C)c2)cn1]

To a N₂ sparged solution of 2-(2-((tert-butyldimethylsilyl)oxy)propan-2-yl)-4-(4,4,5,5-tetramethyl-1,3,2-dioxaborolan-2-yl)pyridine (220 mg, 466 μmol, 80% purity), tert-butyl (R)-(1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)carbamate (300 mg, 502 μmol, 90% purity) and Na₂CO₃ (125 mg, 1.18 mmol) in 1,4-dioxane (8 mL) and water (0.8 mL) was added Pd(dppf)Cl₂ (40 mg, 55 μmol). The resulting mixture was stirred at 90° C. for 16 h under N₂ then diluted with water (20 mL) and extracted with EtOAc (2×15 mL). The organic layers were combined, washed with sat. aq. NaCl (2×20 mL), dried over Na₂SO₄, filtered, and concentrated under reduced pressure. The residue was purified by silica gel chromatography using a gradient of 0-4% MeOH/DCM to obtain the title compound (380 mg, 83%, 72% purity) as a white solid. ES-MS m/z 708.7 (M+H).

The compounds in the following table were prepared as described in Preparation 84 using the appropriate aryl bromide and the appropriate boron ester or boronic acid. Reactants can be added in different orders or in differing equivalency, reaction times (5-32 h) and temperatures (90-100° C.) may vary, and differing methods can be used to work up or purify the compounds, which would be apparent to one skilled in the art.

TABLE 7

| | | | ES-MS |
|---|---|---|---|
| Prep | Chemical Name | Structure | m/z |
| 85^{a} | tert-Butyl (R)-(1-((2-methyl- 1-(5-(trifluoromethyl)-1′H,2H- [3,4′-bipyrazol]-2-yl)propan- 2-yl)amino)-3-(1-methyl-1H- pyrazol-4-yl)-1-oxopropan-2- yl)carbamate | 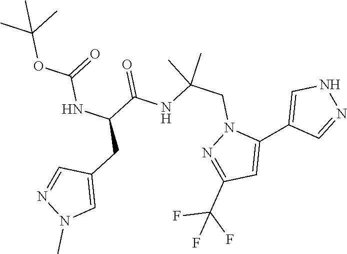 [SMILES: Cn1cc(C[C@@H](NC(=O)OC(C)(C)C)C(=O)NC(C)(C)Cn2nc(C(F)(F)F)cc2-c2cn[nH]c2)cn1] | 525.4 (M + H) |
| 86^{b} | tert-Buty l(R)-(1-((2-methyl- 1-(5′-(trifluoromethyl)- 1H,2′H-[3,3′-bipyrazol]-2′- yl)propan-2-yl)amino)-3-(1- methyl-1H-pyrazol-4-yl)-1- oxopropan-2-yl)carbamate | 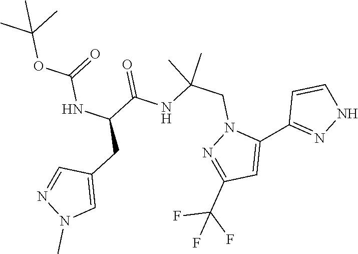 [SMILES: Cn1cc(C[C@@H](NC(=O)OC(C)(C)C)C(=O)NC(C)(C)Cn2nc(C(F)(F)F)cc2-c2cc[nH]n2)cn1] | 525.4 (M + H) |
| 87^{c} | tert-Butyl ((2R)-3-(1- (difluoromethyl)-1H-pyrazol- 4-yl)-1-((2-methyl-1-(5-(2- (tetrahydro-2H-pyran-2-yl)- 2H-1,2,3-triazol-4-yl)-3- (trifluoromethyl)-1H-pyrazol- 1-yl)propan-2-yl)amino)-1- oxopropan-2-yl)carbamate | 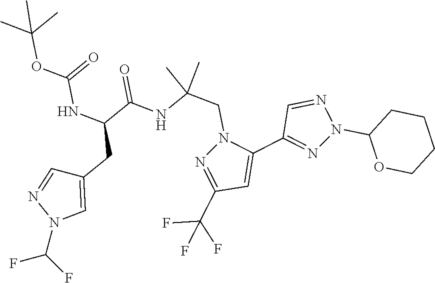 [SMILES: CC(C)(Cn1nc(C(F)(F)F)cc1-c1cnn(C2CCCCO2)n1)NC(=O)[C@@H](Cc1cnn(C(F)F)c1)NC(=O)OC(C)(C)C] | 646.2 (M + H) |

^{a}3 eq boron ester, 100° C., 32 h
^{b}3 eq boron ester, 100° C., 16 h, purification with 0-90% EtOAc/PE.
^{c}90° C., 5 h.

### Preparation 88

### tert-Butyl (R)-(1-((1-(5-(2-(2-hydroxypropan-2-yl)pyridin-4-yl)-3-(trifluoromethyl)-1H-pyrazol-1-yl)-2-methylpropan-2-yl)amino)-3-(1-methyl-1H-pyrazol-4-yl)-1-oxopropan-2-yl)carbamate

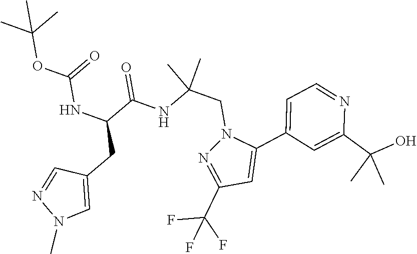 [SMILES: Cn1cc(C[C@@H](NC(=O)OC(C)(C)C)C(=O)NC(C)(C)Cn2nc(C(F)(F)F)cc2-c2ccnc(C(C)(C)O)c2)cn1]

A 0° C. solution of tert-butyl (R)-(1-((1-(5-(2-(2-((tert-butyldimethylsilyl)oxy)propan-2-yl)pyridin-4-yl)-3-(trifluoromethyl)-1H-pyrazol-1-yl)-2-methylpropan-2-yl)amino)-3-(1-methyl-1H-pyrazol-4-yl)-1-oxopropan-2-yl)carbamate (370 mg, 376 μmol) in THF (3 mL) was treated with 1M tetrabutylammonium fluoride in THF (1 mL, 1 mmol), and the mixture was stirred at 15° C. for 2 days. The reaction mixture was quenched with water (20 mL) and extracted with EtOAc (2×10 mL). The organic layers were combined, washed with sat. aq. NaCl (2× 10 mL), dried over Na₂SO₄, filtered, and concentrated under reduced pressure. The residue was purified by silica gel chromatography using a gradient of 0-8% MeOH in DCM to obtain the title compound (180 mg, 79%) as a white solid. ES-MS m z 594.2 (M+H).

### Preparation 89

### (R)—N-(1-(5-(2H-1,2,3-Triazol-4-yl)-3-(trifluoromethyl)-1H-pyrazol-1-yl)-2-methylpropan-2-yl)-2-amino-3-(1-(difluoromethyl)-1H-pyrazol-4-yl)propanamide hydrochloride

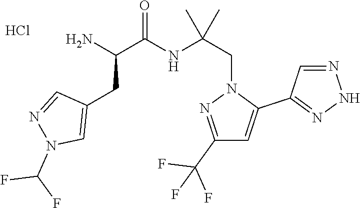 [SMILES: CC(C)(Cn1nc(C(F)(F)F)cc1-c1cn[nH]n1)NC(=O)[C@H](N)Cc1cnn(C(F)F)c1 ; Cl]

A solution of tert-butyl ((2R)-3-(1-(difluoromethyl)-1H-pyrazol-4-yl)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-1-oxopropan-2-yl)carbamate (150 mg, 204 μmol, ˜88% purity) in MeOH (3 mL) was treated with 2 M HCl in MeOH (2.0 mL, 4.0 mmol). The resulting mixture was stirred at 25° C. for 12 h then concentrated under reduced pressure to obtain quantitative yield of the title compound (140 mg, ˜70% purity) as a yellow solid. ES-MS m/z 462.2 (M+H).

### Preparation 90

### Benzyl (R)-(1-((2-methyl-1-(1′-methyl-5-(trifluoromethyl)-1′H,2H-[3,4′-bipyrazol]-2-yl)propan-2-yl)amino)-3-(1-methyl-1H-pyrazol-4-yl)-1-oxopropan-2-yl)carbamate

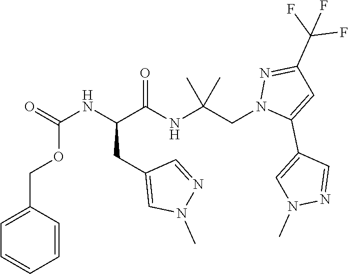 [SMILES: Cn1cc(C[C@@H](NC(=O)OCc2ccccc2)C(=O)NC(C)(C)Cn2nc(C(F)(F)F)cc2-c2cnn(C)c2)cn1]

A mixture of 1-methyl-4-(4,4,5,5-tetramethyl-1,3,2-dioxaborolan-2-yl)-1H-pyrazole (102 mg, 490 μmol), benzyl (R)-(1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)carbamate (140 mg, 245 μmol), and K₃PO₄ (156 mg, 735 μmol) in DME (1.2 mL) was sparged with argon for 2 min. Pd(PPh₃)₄ (28.3 mg, 24.5 μmol) was added, and argon degassing was continued for 3 min. After stirring at 95° C. overnight, the reaction mixture was cooled and treated with additional 1-methyl-4-(4,4,5,5-tetramethyl-1,3,2-dioxaborolan-2-yl)-1H-pyrazole (102 mg, 490 μmol), K₃PO₄ (156 mg, 735 μmol) and Pd(PPh₃)₄ (28.3 mg, 24.5 μmol). Resumed stirring at 95° C. for 4 h. The reaction mixture was cooled and partitioned between water (10 mL) and EtOAc (15 mL). The aq. layer was extracted with EtOAc (3×15 mL). The organic layers were combined, washed with water (10 mL) then sat. aq. NaCl (10 mL), dried over Na₂SO₄, filtered through a pad of diatomaceous earth, and concentrated under reduced pressure. The residue was purified by silica gel chromatography using a gradient of 0-100% (3:1 EtOAc:EtOH) in heptane to obtain a quantitative yield of the title compound as a yellow gel. ES-MS m/z 573.4 (M+H).

### Preparation 91

### tert-Butyl (R)-(3-(imidazo[1,2-a]pyrimidin-3-yl)-1-((2-methyl-1-(1′-methyl-5-(trifluoromethyl)-1′H,2H-[3,4′-bipyrazol]-2-yl)propan-2-yl)amino)-1-oxopropan-2-yl)carbamate

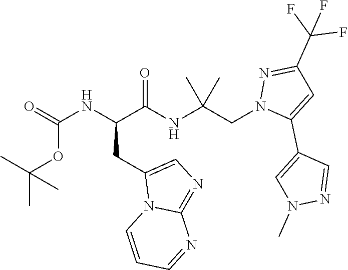 [SMILES: Cn1cc(-c2cc(C(F)(F)F)nn2CC(C)(C)NC(=O)[C@@H](Cc2cnc3ncccn23)NC(=O)OC(C)(C)C)cn1]

The title compound was prepared as described in Preparation 90 using tert-butyl (R)-(1-((1-(5-bromo-3-(trifluoromethyl)-1H-pyrazol-1-yl)-2-methylpropan-2-yl)amino)-3-(imidazo[1,2-a]pyrimidin-3-yl)-1-oxopropan-2-yl)carbamate. ES-MS m/z 576.2 (M+H).

### Preparation 92

### tert-Butyl (R)-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)carbamate

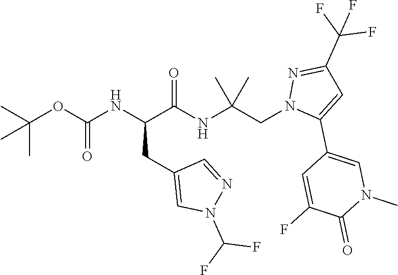 [SMILES: Cn1cc(-c2cc(C(F)(F)F)nn2CC(C)(C)NC(=O)[C@@H](Cc2cnn(C(F)F)c2)NC(=O)OC(C)(C)C)cc(F)c1=O]

To a mixture of tert-butyl (R)-(1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate (2.05 g, 3.58 mmol), 3-fluoro-1-methyl-5-(4,4,5,5-tetramethyl-1,3,2-dioxaborolan-2-yl)pyridin-2(1H)-one (900 mg, 3.56 mmol), K₂CO₃ (1.47 g, 10.6 mmol) and Pd(dppf)Cl₂·CH₂Cl₂ (581 mg, 711 μmol) under N₂ was added N₂ sparged 4:1 1,4-dioxane/water (20 mL). The resulting mixture was stirred at 90° C. for 3.5 h, then cooled to RT and filtered. The filtrate was concentrated under reduced pressure and the residue was purified by silica gel chromatography using a gradient of 0-100% EtOAc/cyclohexane to obtain the title compound (1.25 g, 57%) as a yellow solid. ES-MS m/z 520.2 (M-BOC+H), 642.2 (M+Na).

### Preparation 93

### (R)-(1-(2-(2-((tert-Butoxycarbonyl)amino)-3-(1-methyl-1H-pyrazol-4-yl)propanamido)-2-methylpropyl)-3-(trifluoromethyl)-1H-pyrazol-5-yl) boronic acid

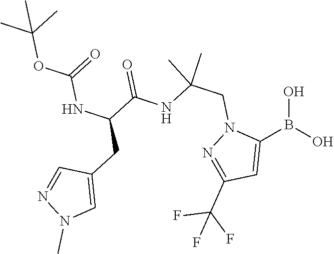 [SMILES: Cn1cc(C[C@@H](NC(=O)OC(C)(C)C)C(=O)NC(C)(C)Cn2nc(C(F)(F)F)cc2B(O)O)cn1]

The title compound was prepared as described in Preparation 20 using tert-butyl (R)-(1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)carbamate and purifying on silica with 0-20% (0.1% FA/MeOH) in DCM. ES-MS m/z 503.3 (M+H).

### Preparation 94

### tert-Butyl (R)-(1-((1-(5-(1,2,4-thiadiazol-3-yl)-3-(trifluoromethyl)-1H-pyrazol-1-yl)-2-methylpropan-2-yl)amino)-3-(1-methyl-1H-pyrazol-4-yl)-1-oxopropan-2-yl)carbamate

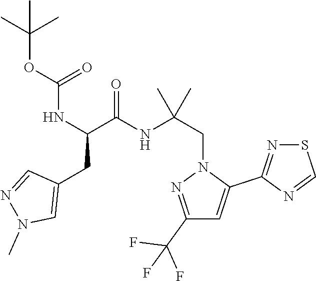 [SMILES: Cn1cc(C[C@@H](NC(=O)OC(C)(C)C)C(=O)NC(C)(C)Cn2nc(C(F)(F)F)cc2-c2ncsn2)cn1]

A solution of (R)-(1-(2-(2-((tert-butoxycarbonyl)amino)-3-(1-methyl-1H-pyrazol-4-yl)propanamido)-2-methylpropyl)-3-(trifluoromethyl)-1H-pyrazol-5-yl) boronic acid (100 mg, 195 μmol) in 1,4-dioxane (5 mL) and water (0.5 mL) was treated with 3-bromo-[1,2,4]thiadiazole (42.3 mg, 254 μmol), Na₂CO₃ (62.7 mg, 585 μmol) and methanesulfonato (2-dicyclohexylphosphino-2′,6′-bis(dimethylamino)-1,1′-biphenyl) (2′-amino-1,1′-biphenyl-2-yl)palladium(II), CPhos Pd G3 (CAS #1447963-73-6, 31.8 mg, 39.0 μmol). The resulting mixture was purged with N₂ and stirred at 80° C. for 16 h under N₂. The reaction mixture was concentrated under reduced pressure, and the residue was purified by silica gel chromatography using a gradient of 0-7% MeOH in DCM to obtain the title compound (82 mg, 47%, 61% purity) as a brown solid. ES-MS m/z 543.3 (M+H).

### Preparation 95

### (R)-2-Amino-N-(1-(4-fluoro-5′-(trifluoromethyl)-1H,2′H-[3,3′-bipyrazol]-2′-yl)-2-methylpropan-2-yl)-3-(1-methyl-1H-pyrazol-4-yl)propanamide hydrochloride

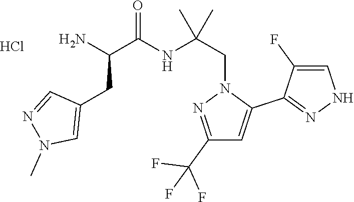 [SMILES: Cl ; Cn1cc(C[C@@H](N)C(=O)NC(C)(C)Cn2nc(C(F)(F)F)cc2-c2n[nH]cc2F)cn1]

A 0° C. solution of 3-bromo-4-fluoro-1H-pyrazole (500 mg, 3.03 mmol) in DMF (8 mL) was treated with NaH (60 wt. % in mineral oil, 145 mg, 3.64 mmol). After stirring at 0° C. for 30 min, 2-(trimethylsilyl) ethoxymethyl chloride (644 μL, 3.64 mmol) was added. The resulting mixture was stirred at 20° C. for 1 h then diluted with EtOAc (30 mL) and water (20 mL). The organic layer was washed with water (2×20 mL), dried over Na₂SO₄, filtered, and concentrated under reduced pressure. The residue was purified by silica gel chromatography using a gradient of 0-3% EtOAc/PE, to give a mixture of regioisomers 3-bromo-4-fluoro-1-((2-(trimethylsilyl) ethoxy)methyl)-1H-pyrazole and 5-bromo-4-fluoro-1-((2-(trimethylsilyl) ethoxy)methyl)-1H-pyrazole (560 mg, 62%) as a colorless oil. ES-MS m z 295,297 (M+H).

A portion of the regioisomeric mixture (209 mg, 351 μmol) was combined with (R)-(1-(2-(2-((tert-butoxycarbonyl)amino)-3-(1-methyl-1H-pyrazol-4-yl)propanamido)-2-methylpropyl)-3-(trifluoromethyl)-1H-pyrazol-5-yl) boronic acid (180 mg, 351 μmol) and Na₂CO₃ (74.4 mg, 702 μmol) in 1,4-dioxane (4 mL) and water (0.4 mL), and XPhos Pd G3 (59.5 mg, 70.2 μmol) was added in a glovebox. After stirring at 90° C. for 16 h under N₂, the reaction mixture was diluted with EtOAc (10 mL) and filtered. The resulting filtrate was concentrated under reduced pressure. The residue was purified by silica gel chromatography using a gradient of 0-3% MeOH/DCM to give a mixture of regioisomers, tert-butyl ((2R)-1-((1-(4′-fluoro-5-(trifluoromethyl)-2′-((2-(trimethylsilyl) ethoxy)methyl)-2H,2′H-[3,3′-bipyrazol]-2-yl)-2-methylpropan-2-yl)amino)-3-(1-methyl-1H-pyrazol-4-yl)-1-oxopropan-2-yl)carbamate and tert-butyl (R)-(1-((1-(4-fluoro-5′-(trifluoromethyl)-1-((2-(trimethylsilyl) ethoxy)methyl)-1H,2′H-[3,3′-bipyrazol]-2′-yl)-2-methylpropan-2-yl)amino)-3-(1-methyl-1H-pyrazol-4-yl)-1-oxopropan-2-yl)carbamate (138 mg, 45%, 77% purity) as a yellow oil. ES-MS m/z 695.4 (M+Na).

The mixture of regioisomers was dissolved in 1,4-dioxane (1 mL) and treated with 2M HCl in 1,4-dioxane (2.00 mL, 4.00 mmol). The resulting mixture was stirred at 20° C. for 16 h then concentrated to obtain the title compound (110 mg, 95%, 65% purity) as a yellow solid. ES-MS m/z 443.3 (M+H).

### Preparation 96

### (R)—N-(2-Methyl-1-(5-(1-methyl-6-oxo-1,6-dihydropyridin-3-yl)-3-(trifluoromethyl)-1H-pyrazol-1-yl)propan-2-yl)-3-(1-methyl-1H-pyrazol-4-yl)-2-(methylamino)propanamide

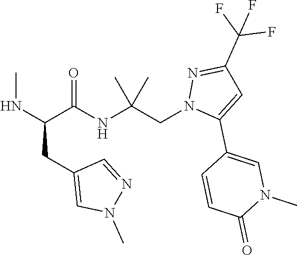 [SMILES: CN[C@H](Cc1cnn(C)c1)C(=O)NC(C)(C)Cn1nc(C(F)(F)F)cc1-c1ccc(=O)n(C)c1]

To a mixture of benzyl (R)-(1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)(methyl)carbamate (1.50 g, 2.56 mmol), 1-methyl-5-(4,4,5,5-tetramethyl-1,3,2-dioxaborolan-2-yl)pyridin-2(1H)-one (1.51 g, 6.41 mmol), Pd(dppf)Cl₂·CH₂Cl₂ (CAS #95464 May 4, 314 mg, 384 μmol) and K₂CO₃ (1.06 g, 7.69 mmol) under N₂ was added 1,4-dioxane (13 mL) and water (4.3 mL). After sparging with N₂ for 5 min, the reaction vessel was sealed, and the mixture was stirred for 30 min at 95° C., then 24 h at 37° C., followed by 22 h at 55° C. The resulting mixture was cooled to RT and diluted with 0.1M aq. HCl (75 mL) and EtOAc (60 mL). The layers were separated, and the organic layer was extracted with 0.1M aq. HCl (4×50 mL). The aq. layers were combined, basified with 2M aq. LiOH, and extracted with EtOAc (6×50 mL). The organic layers were combined, dried over Na₂SO₄, filtered, and concentrated under reduced pressure to obtain the title compound (1.23 g, 85%, 85% purity) as a solid. ES-MS m/z 480.2 (M+H).

### Preparation 97

### tert-Butyl 2-(2-((tert-butoxycarbonyl)amino)-2-methylpropyl) hydrazine-1-carboxylate

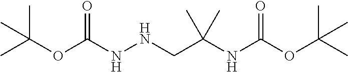 [SMILES: CC(C)(CNNC(=O)OC(C)(C)C)NC(=O)OC(C)(C)C]

To a solution of tert-butyl (2-methyl-1-oxopropan-2-yl)carbamate (9.0 g, 48 mmol) in MeOH (100 mL) was added tert-butyl hydrazinecarboxylate (6.4 g, 48 mmol) and HOAc (8.3 mL, 140 mmol). The mixture was stirred at RT for 2 h, then NaBH₃CN (4.5 g, 72 mmol) was added. After stirring at 25° C. for 16 h, the reaction mixture was concentrated under reduced pressure and diluted with water (100 mL). Na₂CO₃ was added to adjusted to pH-8, and the mixture was extracted with EtOAc (2×100 mL). The organic layers were combined, washed with sat. aq. NaCl (100 mL), dried over Na₂SO₄, filtered, and concentrated under reduced pressure. The residue was purified by silica gel chromatography using a gradient of 0-10% EtOAc/PE to obtain the title compound (13.2 g, 86%) as a white solid. ES-MS m/z 304.2 (M+H).

### Preparation 98

### 1-(2-Amino-2-methylpropyl)-3-(difluoromethyl)-1H-pyrazol-5-ol formate

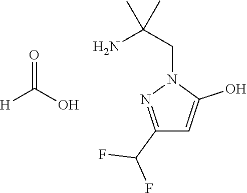

A solution of tert-butyl 2-(2-((tert-butoxycarbonyl)amino)-2-methylpropyl) hydrazine-1-carboxylate (13.2 g, 41.3 mmol) in MeOH (50 mL) was treated with 2M HCl in MeOH (50 mL, 100 mmol). The resulting mixture was stirred at 25° C. for 16 h then concentrated under reduced pressure to give 1-hydrazineyl-2-methylpropan-2-amine hydrochloride (8.5 g, 92%) as a white solid. A portion of this solid (1.57 g, 14.4 mmol) was added to a solution of ethyl 4,4-difluoro-3-oxobutanoate (2.00 g, 12.0 mmol) and FA (1.36 mL, 36.1 mmol) in MTBE (20 mL) and water (5 mL) at 5° C. The cooling bath was removed, and the reaction mixture was stirred at 22° C. for 24 h, then concentrated under reduced pressure. The residue was purified by reversed phase prep-HPLC (Xtimate® C18, 40×150 mm, 10 μm), eluting with a gradient of 0-20% ACN in water (with 0.1% FA) to obtain the title compound (3.6 g, 40%, 34% purity) as a yellow oil. ES-MS (m z) 205.9 (M+H).

### Preparation 99

### tert-Butyl (R)-(1-((1-(3-(difluoromethyl)-5-hydroxy-1H-pyrazol-1-yl)-2-methylpropan-2-yl)amino)-3-(1-methyl-1H-pyrazol-4-yl)-1-oxopropan-2-yl)carbamate

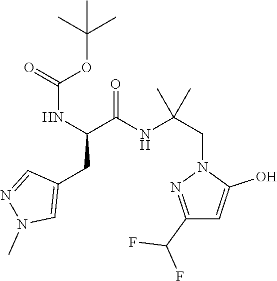 [SMILES: Cn1cc(C[C@@H](NC(=O)OC(C)(C)C)C(=O)NC(C)(C)Cn2nc(C(F)F)cc2O)cn1]

To a solution of 1-(2-amino-2-methylpropyl)-3-(difluoromethyl)-1H-pyrazol-5-ol (700 mg, 1.16 mmol, 34% purity) in DMA (2 mL) was added (R)-2-((tert-butoxycarbonyl)amino)-3-(1-methyl-1H-pyrazol-4-yl)propanoic acid (263 mg, 0.93 mmol), HATU (882 mg, 2.32 mmol) and DIEA (808 μL, 4.64 mmol) at 25° C. After stirring for 3 h, the reaction mixture was diluted with water (50 mL) and extracted with EtOAc (3×35 mL). The organic layers were combined, washed with water (3×40 mL), dried over Na₂SO₄, filtered, and concentrated under reduced pressure. The residue was purified by silica gel chromatography using a gradient of 0-7% DCM/MeOH to obtain the title compound (334 mg, 54%, 85% purity) as a yellow oil. ES-MS (m z) 457.2 (M+H).

### Preparation 100

### (R)-1-(2-(2-((tert-Butoxycarbonyl)amino)-3-(1-methyl-1H-pyrazol-4-yl)propanamido)-2-methylpropyl)-3-(difluoromethyl)-1H-pyrazol-5-yl trifluoromethanesulfonate

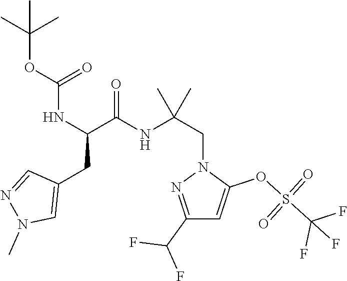 [SMILES: Cn1cc(C[C@@H](NC(=O)OC(C)(C)C)C(=O)NC(C)(C)Cn2nc(C(F)F)cc2OS(=O)(=O)C(F)(F)F)cn1]

A solution of tert-butyl (R)-(1-((1-(3-(difluoromethyl)-5-hydroxy-1H-pyrazol-1-yl)-2-methylpropan-2-yl)amino)-3-(1-methyl-1H-pyrazol-4-yl)-1-oxopropan-2-yl)carbamate (357 mg, 665 μmol, 85% purity) and phenyl triflimide (475 mg, 1.33 mmol) in THF (5 mL) was treated with potassium tert-butoxide (112 mg, 1.0 mmol) at 30° C. The reaction mixture was stirred at 30° C. for 12 h, then concentrated under reduced pressure. The residue was purified by silica gel chromatography using a gradient of 0-20% MeOH in DCM to give the title compound (358 mg, 78%, 85% purity) as a yellow oil. ES-MS (m z) 589.1 (M+H).

### Preparation 101

### tert-Butyl ((2R)-1-((1-(3-(Difluoromethyl)-5-(2-(tetrahydro-2H-pyran-2-yl)-2H-1,2,3-triazol-4-yl)-1H-pyrazol-1-yl)-2-methylpropan-2-yl)amino)-3-(1-methyl-1H-pyrazol-4-yl)-1-oxopropan-2-yl)carbamate

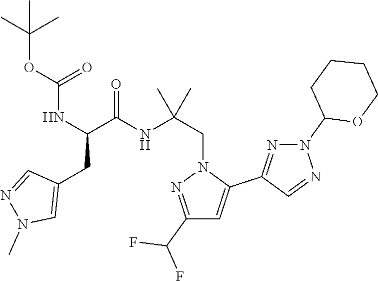 [SMILES: Cn1cc(C[C@@H](NC(=O)OC(C)(C)C)C(=O)NC(C)(C)Cn2nc(C(F)F)cc2-c2cnn(C3CCCCO3)n2)cn1]

To a mixture of (R)-1-(2-(2-((tert-butoxycarbonyl)amino)-3-(1-methyl-1H-pyrazol-4-yl)propanamido)-2-methylpropyl)-3-(difluoromethyl)-1H-pyrazol-5-yl trifluoromethanesulfonate (328 mg, 474 μmol, 85% purity), 2-(tetrahydro-2H-pyran-2-yl)-4-(4,4,5,5-tetramethyl-1,3,2-dioxaborolan-2-yl)-2H-1,2,3-triazole (145 mg, 521 μmol) and Na₂CO₃ (100 mg, 947 μmol) in 1,4-dioxane (5 mL) and water (0.3 mL) was added Pd(dppf)Cl₂ (69.3 mg, 94.7 μmol). The mixture was stirred at 90° C. under N₂ for 16 h. The reaction mixture was poured into water (30 mL) and extracted with EtOAc (3× 20 mL). The organic layers were combined, washed with sat. aq. NaCl (15 mL), dried over Na₂SO₄, filtered, and concentrated under reduced pressure. The residue was purified by silica gel chromatography using a gradient of 0-20% MeOH in DCM, to give the title compound (140 mg, 49%) as a yellow solid. ES-MS m/z 592.3 (M+H).

### Preparation 102

### (R)-2-Amino-N-(1-(3-(difluoromethyl)-5-(2H-1,2,3-triazol-4-yl)-1H-pyrazol-1-yl)-2-methylpropan-2-yl)-3-(1-methyl-1H-pyrazol-4-yl)propanamide hydrochloride

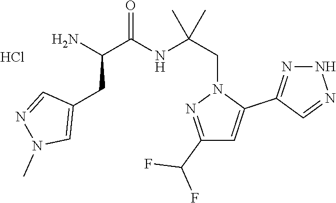 [SMILES: Cl ; Cn1cc(C[C@@H](N)C(=O)NC(C)(C)Cn2nc(C(F)F)cc2-c2cn[nH]n2)cn1]

To a solution of tert-butyl ((2R)-1-((1-(3-(difluoromethyl)-5-(2-(tetrahydro-2H-pyran-2-yl)-2H-1,2,3-triazol-4-yl)-1H-pyrazol-1-yl)-2-methylpropan-2-yl)amino)-3-(1-methyl-1H-pyrazol-4-yl)-1-oxopropan-2-yl)carbamate (140 mg, 234 μmol) in MeOH (3 mL) was added 2 M HCl in 1,4-dioxane (3 mL, 6 mmol) at 25° C. After stirring at 30° C. for 3 h, the mixture was concentrated under reduced pressure to obtain the title compound (164 mg, 95%, 60% purity) as a yellow solid. ES-MS m/z 408.1 (M+H).

### Preparation 103

### (R)-2-Amino-N-(2-methyl-1-(1′-methyl-5-(trifluoromethyl)-1′H,2H-[3,4′-bipyrazol]-2-yl)propan-2-yl)-3-(1-methyl-1H-pyrazol-4-yl)propanamide trifluoroacetate

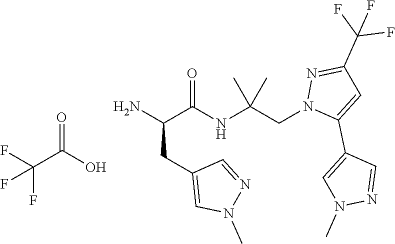 [SMILES: O=C(O)C(F)(F)F ; Cn1cc(C[C@@H](N)C(=O)NC(C)(C)Cn2nc(C(F)(F)F)cc2-c2cnn(C)c2)cn1]

A solution of benzyl (R)-(1-((2-methyl-1-(1′-methyl-5-(trifluoromethyl)-1′H,2H-[3,4′-bipyrazol]-2-yl)propan-2-yl)amino)-3-(1-methyl-1H-pyrazol-4-yl)-1-oxopropan-2-yl)carbamate (140.0 mg, 244.5 μmol) in TFA (2.5 mL) was stirred at 45° C. for 33 h. The reaction mixture was concentrated under a stream of N₂ to obtain a quantitative yield of the title compound as yellow gel. ES-MS m/z 439.4 (M+H).

### Preparation 104

### (R)-2-Amino-N-1-(5-cyclopropyl-3-(trifluoromethyl)-1H-pyrazol-1-yl)-2-methylpropan-2-yl)-3-(1-methyl-1H-pyrazol-4-yl)propanamide trifluoroacetate

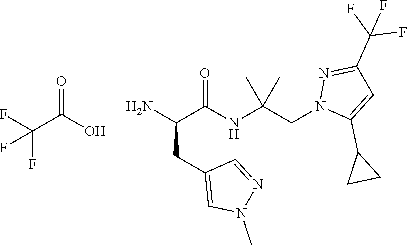 [SMILES: O=C(O)C(F)(F)F ; Cn1cc(C[C@@H](N)C(=O)NC(C)(C)Cn2nc(C(F)(F)F)cc2C2CC2)cn1]

The title compound was prepared as described in Preparation 103 using benzyl (R)-(1-((1-(5-cyclopropyl-3-(trifluoromethyl)-1H-pyrazol-1-yl)-2-methylpropan-2-yl)amino)-3-(1-methyl-1H-pyrazol-4-yl)-1-oxopropan-2-yl)carbamate. ES-MS m/z 399.2 (M+H).

### Preparation 105

### (R)-2-Amino-N-(1-(5-bromo-3-(trifluoromethyl)-1H-pyrazol-1-yl)-2-methylpropan-2-yl)-3-(1-methyl-1H-pyrazol-4-yl)propanamide

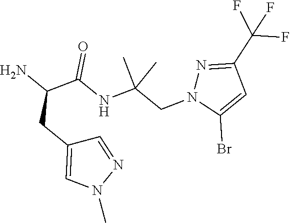 [SMILES: Cn1cc(C[C@@H](N)C(=O)NC(C)(C)Cn2nc(C(F)(F)F)cc2Br)cn1]

A mixture of benzyl (R)-(1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)carbamate (9.9 g, 17 mmol) in TFA (100 mL) was stirred at 35° C. for 3 days. After concentrating under reduced pressure, the oily residue was cooled at 0° C., diluted with DCM (500 mL) and basified with ice-cold 1M aq. NaOH to ˜pH 8. The layers were separated, and the aq. layer was extracted with 3:1 CHCl₃/IPA (500 mL). The organic layers were combined, dried over Na₂SO₄, filtered, and concentrated under reduced pressure. The residue was purified by silica gel chromatography using a gradient of 0-5% 7M NH₃/MeOH in DCM. The mixed fractions were repurified by silica gel chromatography using a gradient of 50 to 100% EA/cyclohexane then switching to 5% 7M NH₃/MeOH in DCM. The two batches were combined to obtain the title compound (4.6 g, 65%) as a colorless oil. ES-MS m/z 437,439 (M-tBu+H).

### Preparation 106

### (R)—N-(1-(5-Bromo-3-(trifluoromethyl)-1H-pyrazol-1-yl)-2-methylpropan-2-yl)-3-(1-methyl-1H-pyrazol-4-yl)-2-(methylamino)propanamide hydrochloride

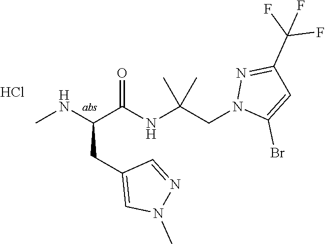

A solution of benzyl (R)-(1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)(methyl)carbamate (3.43 g, 5.86 mmol) in TFA (19.5 mL) was stirred at 55° C. for 2 days. After concentrating under reduced pressure, the residue was partitioned between 3:1 heptane/EtOAc (150 mL) and 0.05 M aq. HCl (150 mL). The organic layer was extracted with 0.05 M aq. HCl (3×50 mL). The aq. layers were combined, neutralized with sat. aq. NaHCO₃ (200 mL), adjusted to pH 10 with sat. aq. Na₂CO₃ and extracted with DCM (4×100 mL). The DCM extracts were combined, dried over Na₂SO₄, and filtered. The filtrate was treated with 4.0 M HCl in 1,4-dioxane (7.3 mL, 29.3 mmol) and the resulting mixture was concentrated under a stream of N₂ at 30° C. to obtain a quantitative yield of the title compound (3.27 g) as an off-white solid. ES-MS m/z 451,453 (M+H).

### Preparation 107

### (R)—N-(2-Methyl-1-(5-(2-oxo-1,2-dihydropyridin-3-yl)-3-(trifluoromethyl)-1H-pyrazol-1-yl)propan-2-yl)-3-(1-methyl-1H-pyrazol-4-yl)-2-(methylamino)propanamide hydrobromide

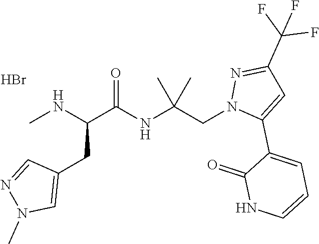 [SMILES: CN[C@H](Cc1cnn(C)c1)C(=O)NC(C)(C)Cn1nc(C(F)(F)F)cc1-c1ccc[nH]c1=O ; Br]

A solution of benzyl (R)-methyl (1-((2-methyl-1-(5-(2-oxo-1,2-dihydropyridin-3-yl)-3-(trifluoromethyl)-1H-pyrazol-1-yl)propan-2-yl)amino)-3-(1-methyl-1H-pyrazol-4-yl)-1-oxopropan-2-yl)carbamate (122.6 mg, 204.5 μmol) in DCM (1.4 mL) was treated with 33 wt. % HBr in HOAc (269 μL, 1.64 mmol) dropwise. The resulting mixture was stirred at 40° C. for 2 h then concentrated under a stream of N₂. EtOAc (5 mL) was added to the residue, and the mixture was sonicated for 2 min. Heptane (5 mL) was added, and the mixture sonicated an additional 2 min. The mixture was concentrated under reduced pressure to obtain a quantitative yield of the title compound (155.6 mg) as a light tan powder. ES-MS m/z 466.2 (M+H).

### Preparation 108

### (R)—N-(2-Methyl-1-(5-(6-oxo-1,6-dihydropyridin-2-yl)-3-(trifluoromethyl)-1H-pyrazol-1-yl)propan-2-yl)-3-(1-methyl-1H-pyrazol-4-yl)-2-(methylamino)propanamide hydrobromide

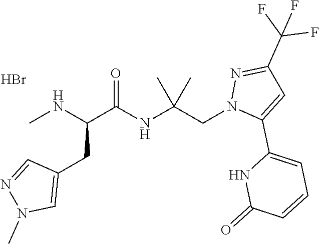 [SMILES: CN[C@H](Cc1cnn(C)c1)C(=O)NC(C)(C)Cn1nc(C(F)(F)F)cc1-c1cccc(=O)[nH]1 ; Br]

The title compound was prepared as described in Preparation 107 using benzyl (R)-methyl (1-((2-methyl-1-(5-(6-oxo-1,6-dihydropyridin-2-yl)-3-(trifluoromethyl)-1H-pyrazol-1-yl)propan-2-yl)amino)-3-(1-methyl-1H-pyrazol-4-yl)-1-oxopropan-2-yl)carbamate. ES-MS m/z 466.2 (M+H).

### Preparation 109

### (R)-2-Amino-N-(2-methyl-1-(5-(1-methyl-6-oxo-1,6-dihydropyridin-3-yl)-3-(trifluoromethyl)-1H-pyrazol-1-yl)propan-2-yl)-3-(1-methyl-1H-pyrazol-4-yl)propanamide hydrobromide

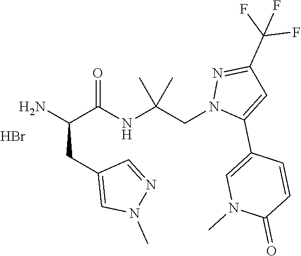 [SMILES: Br ; Cn1cc(C[C@@H](N)C(=O)NC(C)(C)Cn2nc(C(F)(F)F)cc2-c2ccc(=O)n(C)c2)cn1]

To a solution of tert-Butyl (R)-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)carbamate (140 mg, 219 μmol) in DCM (4 mL) was added BBr₃ (150 μL, 1.59 mmol) at 0° C. The mixture was stirred cold for 2 h then combined with a second reaction mixture (31 μmol scale). The combined mixture was suction filtered, and the collected solid was washed with EtOAc to obtain the title compound (140 mg, 65%, ˜64% purity) as a white solid. ES-MS m/z 473.2 (M+H).

### Preparation 110

### (R)-2-amino-N-(2-methyl-1-(4-(trifluoromethyl)-1H-pyrazol-1-yl)propan-2-yl)-3-(1-methyl-1H-pyrazol-4-yl)propanamide

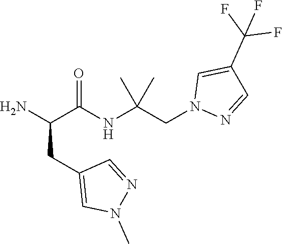 [SMILES: Cn1cc(C[C@@H](N)C(=O)NC(C)(C)Cn2cc(C(F)(F)F)cn2)cn1]

To a mixture of 10% Pd/C (50% wet) (1.5 g, 1.4 mmol) wetted with EtOH (1 mL) and under a blanket of N₂ was added benzyl (R)-(1-((2-methyl-1-(4-(trifluoromethyl)-1H-pyrazol-1-yl)propan-2-yl)amino)-3-(1-methyl-1H-pyrazol-4-yl)-1-oxopropan-2-yl)carbamate (3.88 g, 7.9 mmol) and MeOH (40 mL). The resulting mixture was stirred under 0.31 MPa of H₂ at RT overnight. The reaction was repeated on the same scale. The reaction mixtures were combined by filtering over the same bed of diatomaceous earth, rinsing with EA. The filtrate was concentrated under reduced pressure, and the residue was dissolved in DCM and passed through a hydrophobic frit, rinsing with DCM. The filtrate was concentrated under reduced pressure to give a quantitative yield of the title compound (5.84 g) as a clear, slightly yellow oil. ES-MS m/z 359.2 (M+H).

The compounds in the following table were prepared as described in Preparation 110 from the appropriate benzyl carbamate. Varying H₂ pressures (0.10-0.31 MPa) and reaction times (4 h to overnight) can be used, which would be apparent to one skilled in the art.

TABLE 8

| Prep | Chemical Name | Structure | ES-MS m/z |
|---|---|---|---|
| 111 | (R)-2-Amino-N-(2-methyl-1-(5- (1-methyl-6-oxo-1,6- dihydropyridin-3-yl)-3- (trifluoromethyl)-1H-pyrazol-1- yl)propan-2-yl)-3-(1-methyl-1H- pyrazol-4-yl)propanamide | 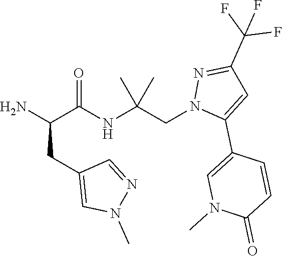 [SMILES: Cn1cc(C[C@@H](N)C(=O)NC(C)(C)Cn2nc(C(F)(F)F)cc2-c2ccc(=O)n(C)c2)cn1] | 466.2 (M + H) |
| 112ª | (R)-2-Amino-N-(2-methyl-1-(5- (2-(tetrahydro-2H-pyran-2-yl)- 2H-1,2,3-triazol-4-yl)-3- (trifluoromethyl)-1H-pyrazol-1- yl)propan-2-yl)-3-(1-methyl-1H- pyrazol-4-yl)propanamide | 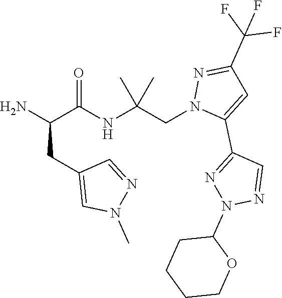 [SMILES: Cn1cc(C[C@@H](N)C(=O)NC(C)(C)Cn2nc(C(F)(F)F)cc2-c2cnn(C3CCCCO3)n2)cn1] | 510.4 (M + H) |
| 113 | (R)-2-Amino-N-(1-(4- fluorophenyl)-2-methylpropan-2- yl)-3-(1-methyl-1H-pyrazol-4- yl)propanamide | 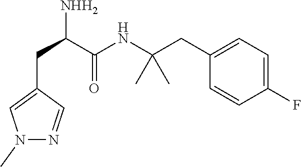 | 319.0 (M + H) |

^{a}catalyst: 20% Pd(OH)₂, 50% water wet

### Preparation 114

### (R)-2-Amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide hydrochloride

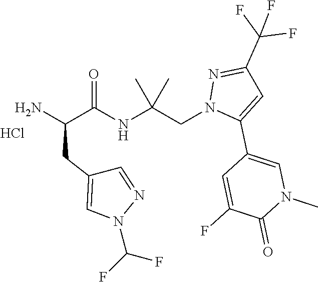 [SMILES: Cl ; Cn1cc(-c2cc(C(F)(F)F)nn2CC(C)(C)NC(=O)[C@H](N)Cc2cnn(C(F)F)c2)cc(F)c1=O]

A solution of tert-butyl (R)-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)carbamate (1.25 g, 2.02 mmol) in MeOH (10 mL) was added 4M HCl in 1,4-dioxane (7.6 mL, 30 mmol). After stirring for 18 h at RT, the mixture was concentrated under a stream of N₂ to obtain a quantitative yield of the title compound (1.1 g) as a yellow solid. ES-MS m/z 520.0 (M+H).

The compounds in the following table were prepared as described in Preparation 114 from the appropriate tert-butyl carbamate. Varying reaction times (15 min-24 h), equivalents of HCl (3-25 eq), and co-solvents (such as EtOAc) can be used, which would be apparent to one skilled in the art. The deprotected amines were isolated as the corresponding hydrochloride salt. The compounds may have been used without determination of the number of equivalents of HCl.

TABLE 9

| Prep | Chemical Name | Structure | ES-MS m/z |
|---|---|---|---|
| 115 | (R)-2-Amino-N-(2-methyl-1- (4-(trifluoromethyl)-1H- pyrazol-1-yl)propan-2-yl)-3-(1- methyl-1H-pyrazol-4- yl)propanamide hydrochloride | 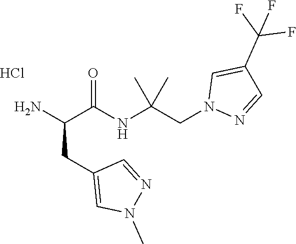 [SMILES: Cl ; Cn1cc(C[C@@H](N)C(=O)NC(C)(C)Cn2cc(C(F)(F)F)cn2)cn1] | 359.1 (M + H) |
| 116 | (R)-2-Amino-N-(1-(5-bromo-3- (trifluoromethyl)-1H-pyrazol- 1-yl)-2-methylpropan-2-yl)-3- (1-methyl-1H-pyrazol-4- yl)propanamide hydrochloride | 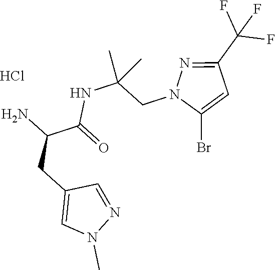 [SMILES: Cl ; Cn1cc(C[C@@H](N)C(=O)NC(C)(C)Cn2nc(C(F)(F)F)cc2Br)cn1] | 437, 439 (M + H) |
| 117 | (R)-N-(1-(5-(2H-1,2,3-Triazol- 4-yl)-3-(trifluoromethyl)-1H- pyrazol-1-yl)-2-methylpropan- 2-yl)-2-amino-3-(1-methyl-1H- pyrazol-4-yl)propanamide hydrochloride | 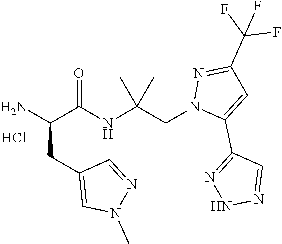 [SMILES: Cl ; Cn1cc(C[C@@H](N)C(=O)NC(C)(C)Cn2nc(C(F)(F)F)cc2-c2cn[nH]n2)cn1] | 426.2 (M + H) |
| 118 | (R)-2-Amino-N-(1-(5-bromo-3- (trifluoromethyl)-1H-pyrazol- 1-yl)-2-methylpropan-2-yl)-3- (1-(difluoromethyl)-1H- pyrazol-4-yl)propanamide hydrochloride | 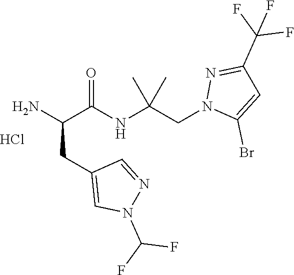 [SMILES: Cl ; CC(C)(Cn1nc(C(F)(F)F)cc1Br)NC(=O)[C@H](N)Cc1cnn(C(F)F)c1] | 473.0 (M + H) |
| 119 | (R)-N-(1-(5-(1H-1,2,3-Triazol- 1-yl)-3-(trifluoromethyl)-1H- pyrazol-1-yl)-2-methylpropan- 2-yl)-2-amino-3-(1-methyl-1H- pyrazol-4-yl)propanamide hydrochloride | 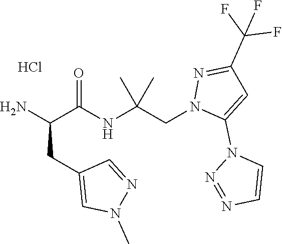 [SMILES: Cn1cc(C[C@@H](N)C(=O)NC(C)(C)Cn2nc(C(F)(F)F)cc2-n2ccnn2)cn1 ; Cl] | 426.4 (M + H) |
| 120 | (R)-N-(1-(5-(2H-1,2,3-Triazol- 2-yl)-3-(trifluoromethyl)-1H- pyrazol-1-yl)-2-methylpropan- 2-yl)-2-amino-3-(1-methyl-1H- pyrazol-4-yl)propanamide hydrochloride | 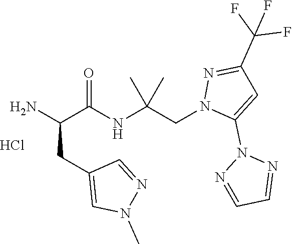 [SMILES: Cl ; Cn1cc(C[C@@H](N)C(=O)NC(C)(C)Cn2nc(C(F)(F)F)cc2-n2nccn2)cn1] | 426.2 (M + H) |
| 121 | Methyl (R)-1-(2-(2-amino-3- (1-methyl-1H-pyrazol-4- yl)propanamido)-2- methylpropyl)-3- (trifluoromethyl)-1H-pyrazole- 5-carboxylate hydrochloride | 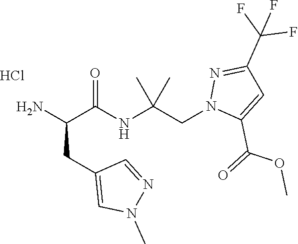 [SMILES: Cl ; COC(=O)c1cc(C(F)(F)F)nn1CC(C)(C)NC(=O)[C@H](N)Cc1cnn(C)c1] | 417.4 (m + H) |
| 122 | (R)-N-(1-(5-(4H-1,2,4-Triazol- 3-yl)-3-(trifluoromethyl)-1H- pyrazol-1-yl)-2-methylpropan- 2-yl)-2-amino-3-(1-methyl-1H- pyrazol-4-yl)propanamide hydrochloride | 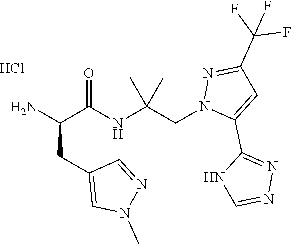 [SMILES: Cl ; Cn1cc(C[C@@H](N)C(=O)NC(C)(C)Cn2nc(C(F)(F)F)cc2-c2nnc[nH]2)cn1] | 426.4 (M + H) |
| 123 | (R)-2-Amino-N-(2-methyl-1- (5-(2-methyl-1H-imidazol-5- yl)-3-(trifluoromethyl)-1H- pyrazol-1-yl)propan-2-yl)-3-(1- methyl-1H-pyrazol-4- yl)propanamide hydrochloride | 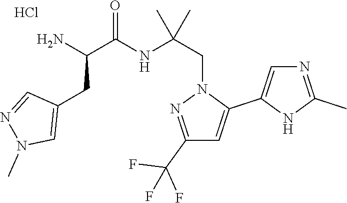 [SMILES: Cc1ncc(-c2cc(C(F)(F)F)nn2CC(C)(C)NC(=O)[C@H](N)Cc2cnn(C)c2)[nH]1 ; Cl] | 439.2 (M + H) |
| 124 | (R)-2-Amino-N-(2-methyl-1- (5-(trifluoromethyl)-1′H,2H- [3,4′-bipyrazol]-2-yl)propan-2- yl)-3-(1-methyl-1H-pyrazol-4- yl)propanamide hydrochloride | 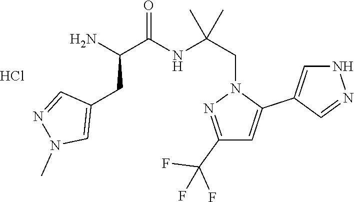 [SMILES: Cl ; Cn1cc(C[C@@H](N)C(=O)NC(C)(C)Cn2nc(C(F)(F)F)cc2-c2cn[nH]c2)cn1] | 425.3 (M + H) |
| 125 | (R)-2-Amino-N-(2-methyl-1- (5′-(trifluoromethyl)-1H,2′H- [3,3′-bipyrazol]-2′-y1)propan-2- yl)-3-(1-methyl-1H-pyrazol-4- yl)propanamide hydrochloride | 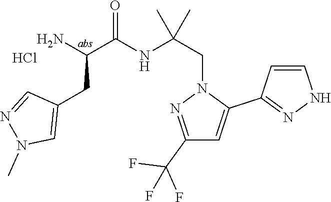 [SMILES: Cn1cc(C[C@@H](N)C(=O)NC(C)(C)Cn2nc(C(F)(F)F)cc2-c2cc[nH]n2)cn1 ; Cl] | 425.1 (M + H) |
| 126 | (R)-2-Amino-N-(2-methyl-1- (5-(5-oxo-4,5-dihydro-1,3,4- thiadiazol-2-yl)-3- (trifluoromethyl)-1H-pyrazol- 1-yl)propan-2-yl)-3-(1-methyl- 1H-pyrazol-4-yl)propanamide hydrochloride | 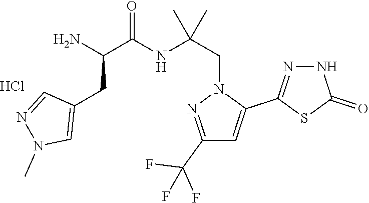 [SMILES: Cl ; Cn1cc(C[C@@H](N)C(=O)NC(C)(C)Cn2nc(C(F)(F)F)cc2-c2n[nH]c(=O)s2)cn1] | 459.2 (M + H) |
| 127 | (R)-2-Amino-N-(2-methyl-1- (5-(1-methyl-6-oxo-1,6- dihydropyridin-3-yl)-3- (trifluoromethyl)-1H-pyrazol- 1-yl)propan-2-yl)-3-(1-methyl- 1H-pyrazol-4-yl)propanamide hydrochloride | 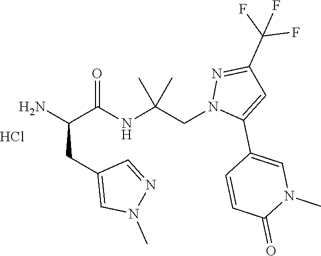 [SMILES: Cl ; Cn1cc(C[C@@H](N)C(=O)NC(C)(C)Cn2nc(C(F)(F)F)cc2-c2ccc(=O)n(C)c2)cn1] | 466.4 (M + H) |
| 128 | (R)-2-Amino-N-(2-methyl-1- (5-(2-oxopyridin-1(2H)-yl)-3- (trifluoromethyl)-1H-pyrazol- 1-yl)propan-2-yl)-3-(1-methyl- 1H-pyrazol-4-yl)propanamide hydrochloride | 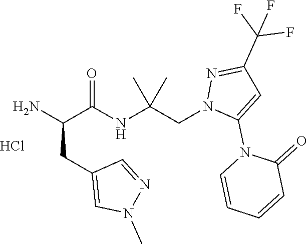 [SMILES: Cl ; Cn1cc(C[C@@H](N)C(=O)NC(C)(C)Cn2nc(C(F)(F)F)cc2-n2ccccc2=O)cn1] | 452.2 (M + H) |
| 129 | (R)-2-Amino-N-(1-(5-(2-(2- hydroxypropan-2-yl)pyridin-4- yl)-3-(trifluoromethyl)-1H- pyrazol-1-yl)-2-methylpropan- 2-yl)-3-(1-methyl-1H-pyrazol- 4-yl)propanamide hydrochloride | 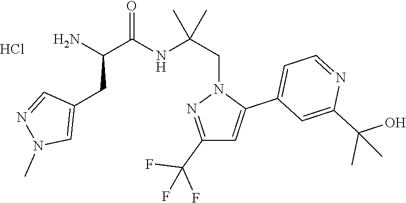 [SMILES: Cl ; Cn1cc(C[C@@H](N)C(=O)NC(C)(C)Cn2nc(C(F)(F)F)cc2-c2ccnc(C(C)(C)O)c2)cn1] | 494.4 (M + H) |
| 130 | (R)-2-Amino-N-(1-(5-bromo-3- (trifluoromethyl)-1H-pyrazol- 1-yl)-2-methylpropan-2-yl)-3- (imidazo[1,2-a]pyrimidin-3- yl)propanamide hydrochloride | 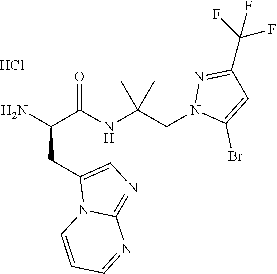 [SMILES: Cl ; CC(C)(Cn1nc(C(F)(F)F)cc1Br)NC(=O)[C@H](N)Cc1cnc2ncccn12] | (⁷⁹Br/⁸¹Br) 474.0/476.0 (M + H) |
| 131 | (R)-2-Amino-3-(imidazo[1,2- a]pyrimidin-3-yl)-N-(2-methyl- 1-(1′-methyl-5- (trifluoromethyl)-1′H,2H-[3,4′- bipyrazol]-2-yl)propan-2- yl)propanamide | 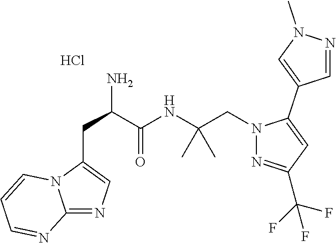 [SMILES: Cn1cc(-c2cc(C(F)(F)F)nn2CC(C)(C)NC(=O)[C@H](N)Cc2cnc3ncccn23)cn1 ; Cl] | 476.2 (M + H) |
| 132 | (R)-2-Amino-N-(1-(5- cyclopropyl-3- (trifluoromethyl)-1H-pyrazol- 1-yl)-2-methylpropan-2-yl)-3- (imidazo[1,2-a]pyrimidin-3- yl)propanamide hydrochloride | 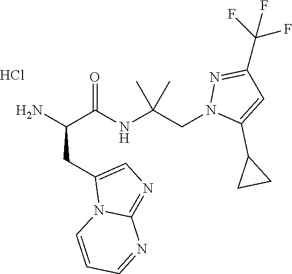 [SMILES: Cl ; CC(C)(Cn1nc(C(F)(F)F)cc1C1CC1)NC(=O)[C@H](N)Cc1cnc2ncccn12] | 436.2 (M + H) |
| 133 | (R)-2-Amino-3-(imidazo[1,2- a]pyrimidin-3-yl)-N-(2-methyl- 1-(4-(trifluoromethyl)-1H- pyrazol-1-yl)propan-2- yl)propanamide hydrochloride | 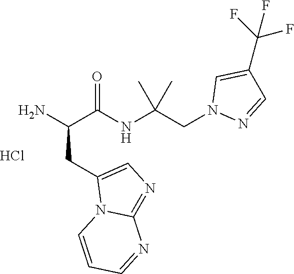 [SMILES: Cl ; CC(C)(Cn1cc(C(F)(F)F)cn1)NC(=O)[C@H](N)Cc1cnc2ncccn12] | 396.2 (M + H) |
| 134 | (R)-2-Amino-3-(imidazo[1,2- a]pyrimidin-3-yl)-N-(2-methyl- 1-(3-(trifluoromethyl)-1H- pyrazol-1-yl)propan-2- yl)propanamide hydrochloride | 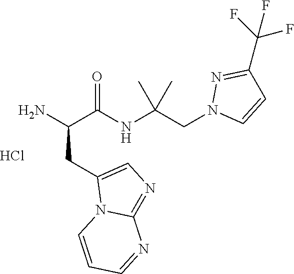 [SMILES: Cl ; CC(C)(Cn1ccc(C(F)(F)F)n1)NC(=O)[C@H](N)Cc1cnc2ncccn12] | 396.2 (M + H) |
| 135 | (R)-2-Amino-N-(1-(4- fluorophenyl)-2-methylpropan- 2-yl)-3-(imidazo[1,2- a]pyrimidin-3-yl)propanamide hydrochloride | 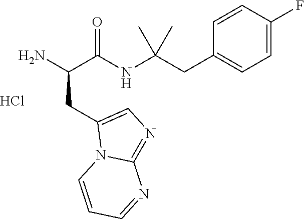 [SMILES: Cl ; CC(C)(Cc1ccc(F)cc1)NC(=O)[C@H](N)Cc1cnc2ncccn12] | 356.2 (M + H) |
| 136 | (R)-2-Amino-N-(1-(5- fluoropyridin-2-yl)-2- methylpropan-2-yl)-3- (imidazo[1,2-a]pyrimidin-3- yl)propanamide hydrochloride | 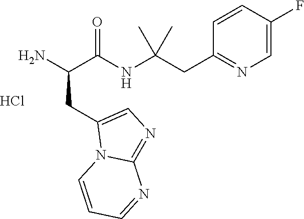 [SMILES: Cl ; CC(C)(Cc1ccc(F)cn1)NC(=O)[C@H](N)Cc1cnc2ncccn12] | 357.2 (M + H) |

### Preparation 137

### ((R)-2-Amino-N-(1-(4-fluorophenyl)-2-methylpropan-2-yl)-3-(1-methyl-1H-pyrazol-4-yl)propanamide hydrochloride

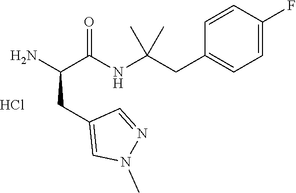 [SMILES: Cl ; Cn1cc(C[C@@H](N)C(=O)NC(C)(C)Cc2ccc(F)cc2)cn1]

To a mixture of ((R)-2-amino-N-(1-(4-fluorophenyl)-2-methylpropan-2-yl)-3-(1-methyl-1H-pyrazol-4-yl)propanamide (70 g, 0.220 mol) in 1,4-dioxane (140 mL) was added 4M HCl in 1,4-dioxane (175 mL) at 0° C. The resulting mixture was allowed to stir at RT for 3 h. The solid was filtered and dried under reduced pressure at 40° C. to obtain a quantitative yield of the title compound (80.0 g) as white solid. ES-MS m/z 319.0 (M+H).

### Preparation 138

### (R)—N-(1-(4-Fluorophenyl)-2-methylpropan-2-yl)-3-(1-methyl-1H-pyrazol-4-yl)-2-((2-nitrophenyl)sulfonamido)propanamide

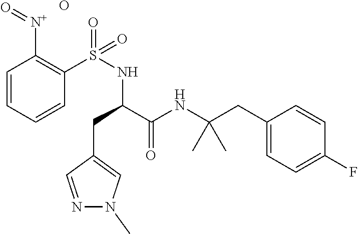

A mixture of (R)-2-amino-N-(1-(4-fluorophenyl)-2-methylpropan-2-yl)-3-(1-methyl-1H-pyrazol-4-yl)propanamide hydrochloride (1.5 g, 3.8 mmol) in DCM (15 mL) was treated with TEA (1.1 mL, 8.0 mmol) and 2-nitrobenzene sulfonyl chloride (1.6 g, 7.1 mmol). The mixture was stirred at RT for 2 h then partitioned between sat. aq. NH₄Cl (50 mL) and DCM (50 mL).

The aq. layer was extracted with DCM (3×50 mL). The organic layers were combined, washed with water (30 mL), then sat. aq. NaCl (30 mL), dried over Na₂SO₄, filtered, and concentrated under reduced pressure to obtain a quantitative yield of the title compound as a yellow gel. ES-MS m/z 504.2 (M+H).

### Preparation 139

### (R)—N-(1-(4-Fluorophenyl)-2-methylpropan-2-yl)-3-(1-methyl-1H-pyrazol-4-yl)-2-((N-methyl-2-nitrophenyl)sulfonamido)propanamide

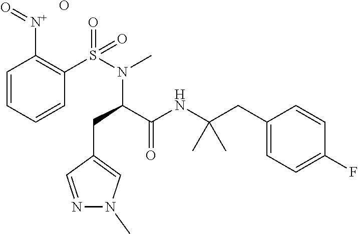

A mixture of (R)—N-(1-(4-fluorophenyl)-2-methylpropan-2-yl)-3-(1-methyl-1H-pyrazol-4-yl)-2-((2-nitrophenyl)sulfonamido)propanamide (2.4 g, 4.8 mmol) in DMF (32 mL) was treated with Cs₂CO₃ (1.7 g, 5.2 mmol). After stirring at RT for 5 min, methyl iodide (0.93 mL, 14 mmol) was added, and the resulting mixture was stirred at RT overnight. The reaction mixture was partitioned between water (50 mL) and DCM (50 mL). The aq. layer was extracted with DCM (3×50 mL). The organic layers were combined, washed with water (3×40 mL), then sat. aq. NaCl (40 mL), dried over Na₂SO₄, filtered, and concentrated under reduced pressure. The residue was purified by silica gel chromatography using a gradient of 0-80% (3:1 EtOAc:EtOH) in heptane to give the title compound (1.5 g, 60%). ES-MS m/z 518.2 (M+H).

### Preparation 140

### (R)—N-(1-(4-Fluorophenyl)-2-methylpropan-2-yl)-3-(1-methyl-1H-pyrazol-4-yl)-2-(methylamino)propanamide

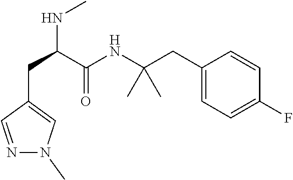 [SMILES: CN[C@H](Cc1cnn(C)c1)C(=O)NC(C)(C)Cc1ccc(F)cc1]

A mixture of (R)—N-(1-(4-fluorophenyl)-2-methylpropan-2-yl)-3-(1-methyl-1H-pyrazol-4-yl)-2-((N-methyl-2-nitrophenyl)sulfonamido)propanamide (100 mg, 193 μmol) and LiOH (19 mg, 773 μmol) in DMF (1.3 mL) was treated with thioglycolic acid (36 mg, 386 μmol). The resulting mixture was stirred at RT for 1 h, then partitioned between sat. aq. NH₄Cl (5 mL) and DCM (10 mL). The aq. layer was extracted with DCM (3×10 mL). The organic layers were combined, washed with water (2×10 mL), then sat. aq. NaCl (5 mL), dried over Na₂SO₄, filtered, and concentrated under reduced pressure. The residue was purified by silica gel chromatography using a gradient of 0-100% (3:1 EtOAc:EtOH) in DCM to obtain the title compound (32.7 mg, 50%). ES-MS: 333.2 m/z (M+H).

### Preparation 141

### 5-Bromo-2-cyclobutyl-2H-pyrazolo[3,4-b]pyridine

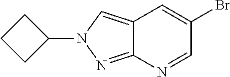 [SMILES: Brc1cnc2nn(C3CCC3)cc2c1]

A mixture of 5-bromo-1H-pyrazolo[3,4-b]pyridine (3.00 g, 15.0 mmol) in NMP (60 mL) was treated with NaH (1.02 g, 25.5 mmol, 60% in oil) in one portion, and the reaction mixture was stirred until gas evolution ceased (˜5 min). Bromocyclobutane (3.53 mL, 37.5 mmol) was added, and the resulting mixture was stirred at 115° C. for 42 h. The reaction mixture was cooled to RT, diluted with water (250 mL), and extracted with EtOAc (3×50 mL). The organic layers were combined, dried over Na₂SO₄, filtered, and concentrated under reduced pressure. The residue was purified by silica gel chromatography using a gradient of 5-50% EtOAc in heptane to obtain the second-eluting (minor) isomer, the title compound (second-eluting minor isomer, 299 mg, 8%) as an off-white solid.

### Preparation 142

### 6-Bromo-2-(trifluoromethyl) imidazo[1,2-a]pyrimidine

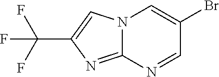 [SMILES: FC(F)(F)c1cn2cc(Br)cnc2n1]

A mixture of 5-bromo-pyrimidin-2-ylamine (8.75 g, 50.3 mmol), DME (100 mL) and 3-bromo-1,1,1-trifluoroacetone (9.41 mL, 90.5 mmol). After stirring at RT for 12 days, the reaction mixture was filtered, and the filter cake was washed with heptane (20 mL) and dried to give a tan solid (17.3 g) which was suspended in EtOH (150 mL). The suspension was stirred at reflux for 21 h. After cooling to RT, the reaction mixture was concentrated under reduced pressure to approximately 25 mL and diluted with heptane (40 mL). The mixture was stirred for 5 min then filtered. The filter cake was vacuum dried to obtain the title compound (7.91 g, 59%) as a light-pink solid. ES-MS m/z 266,268 (M+H).

### Preparation 143

### 6-Bromo-2-methyl-1,8-naphthyridine

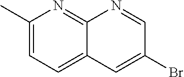

A mixture of sodium tert-butoxide (166 mg, 1.72 mmol), and 1,3-bis(2,6-diisopropylphenyl) imidazol-2-ylidene copper chloride (210 mg, 0.43 mmol) was sealed in an oven-dried vial, purged with N₂, and treated with a solution of 4-hydroxy-4-methylpentan-2-one (1.00 g, 8.61 mmol) and 2-amino-5-bromonicotinaldehyde (1.73 g, 8.61 mmol) in toluene (20 mL). The resulting mixture was stirred at 70° C. for 5 h, then cooled, filtered through a pad of silica, and concentrated under reduced pressure. The residue was purified by silica gel chromatography using a gradient of 40-70% EtOAc in cyclohexane, to give the title compound (750 mg, 39% yield) as a white solid. ES-MS m/z 223,225 (M+H).

### Preparation 144

### 2-Methyl-4-(4,4,5,5-tetramethyl-1,3,2-dioxaborolan-2-yl)-2H-1,2,3-triazole

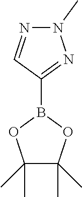

A mixture of 4-bromo-2-methyl-2H-1,2,3-triazole (3.69 g, 22.1 mmol), bis(pinacolato)diboron (6.23 g, 24.3 mmol), KOAc (6.51 g, 66.3 mmol), Pd(dppf)Cl₂·CH₂Cl₂ (2.26 g, 2.77 mmol), and 1,4-dioxane (100 mL) was purged with N₂ and then stirred at 100° C. for 2 h. The reaction mixture was cooled to RT and filtered through a pad of silica (65 g) and rinsing with EtOAc. The filtrate was concentrated under reduced pressure. The residue was purified by silica gel chromatography, eluting with EtOAc in hexanes and co-evaporated with DCM to afford the title product (3.86 g, 80%) as a light brown solid. 1H NMR (CDCl₃): 7.89 (s, 1H), 4.25 (s, 3H), 1.36 (s, 12H).

### Preparation 146

### 2-(2-Methyl-2H-1,2,3-triazol-4-yl)-1,3,6,2-dioxazaborocane

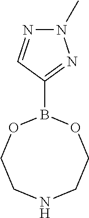

A solution of 2-methyl-4-(4,4,5,5-tetramethyl-1,3,2-dioxaborolan-2-yl)-2H-1,2,3-triazole (21.1 g, 70.7 mmol, 70% purity) in cyclopentyl methyl ether (210 mL) stirring at 55° C. under N₂ was treated with a solution of diethanolamine (7.13 mL, 74.2 mmol) in IPA (21 mL) in portions by pipette over 10 min. Stirring at 55° C. was continued for 2 h, and then reaction mixture was allowed to slowly cool to RT with continued stirring. After ˜5 h, the solid was collected by filtration, washed with 10:1 cyclopentyl methyl ether: IPA (110 mL), and dried overnight in a 40° C. vacuum oven to give the title compound (13.1 g, 93%) as light-grey solid. ES-MS m/z 197.0 (M+H).

### Preparation 147

### Methyl (1S,2S)-2-(4-bromophenyl)cyclopropane-1-carboxylate

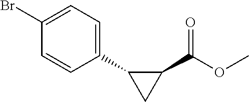

A stirring solution of (1S,2S)-2-(4-bromophenyl)cyclopropane-1-carboxylic acid (prepared as described in US 2011/0201623 A1; 12.0 g, 49.8 mmol) in MeOH (100 mL) at RT was treated with SOCl₂ (3.70 mL, 50.8 mmol) dropwise. The resulting mixture was stirred at 50° C. overnight then concentrated under reduced pressure. The residue was partitioned between EtOAc (40 mL) and sat. aq. NaHCO₃ (100 mL), and the layers were separated. The organic layer was washed with sat. aq. NaCl, dried over Na₂SO₄, filtered, and concentrated under reduced pressure to obtain a quantitative yield of the title compound (12.7 g) as a light-yellow oil. ES-MS m/z 255,257 (M+H).

### Preparation 148

### Methyl (1S,2S)-2-(4-(4,4,5,5-tetramethyl-1,3,2-dioxaborolan-2-yl)phenyl)cyclopropane-1-carboxylate

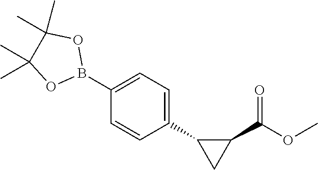

To a mixture of bis(pinacolato) diborane (7.47 g, 29.4 mmol), KOAc (5.77 g, 58.8 mmol) and Pd(dppf)Cl₂·CH₂Cl₂ (1.60 g, 1.96 mmol) under N₂ was added 1,4-dioxane (245 mL) and methyl (1S,2S)-2-(4-bromophenyl)cyclopropane-1-carboxylate (5.00 g, 19.6 mmol). The resulting mixture was sparged with N₂ for 20 min at RT, then stirred at 90° C. overnight under N₂. The reaction mixture was cooled to RT, diluted with EtOAc, and filtered through diatomaceous earth. The filtrate was concentrated under reduced pressure, and the residue was purified by silica gel chromatography using a gradient of 0-50% EtOAc in cyclohexane to obtain the title compound (5.71 g, 96%). ES-MS m/z 303.0 (M+H).

### Preparation 149

### Methyl (1S,2S)-2-(4-(2-methyl-2H-pyrazolo[3,4-b]pyridin-5-yl)phenyl)cyclopropane-1-carboxylate

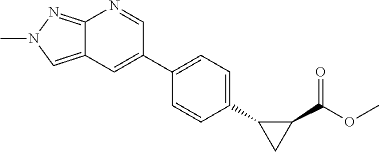

A mixture of 5-bromo-2-methylpyrazolo[3,4-b]pyridine (5.00 g, 23.6 mmol), methyl (1S,2S)-2-(4-(4,4,5,5-tetramethyl-1,3,2-dioxaborolan-2-yl)phenyl)cyclopropane-1-carboxylate (2.85 g, 9.43 mmol), Cs₂CO₃ (11.1 g, 34.0 mmol) and Pd(dppf)Cl₂·CH₂Cl₂ (1.16 g, 1.41 mmol) under N₂ was treated with 1,4-dioxane (240 mL) and water (24 mL), and the resulting mixture was sparged with N₂ for 30 min, then stirred at 90° C. overnight. After concentrating most of the solvent under reduced pressure, the residue was poured into water (200 mL) and sat. aq. NaCl (50 mL). EtOAc (100 mL) was added, and the layers were separated. The aq. layer was extracted with EtOAc (4×100 mL). The organic layers were combined, washed with sat. aq. NaCl, dried over Na₂SO₄, filtered, and concentrated under reduced pressure. The residue was purified by silica gel chromatography using a gradient of 15-100% (3:1 EtOAc:EtOH) in cyclohexane to obtain the title compound (2.10 g, 72%) as a tan solid. ES-MS m/z 308.0 (M+H).

The compounds in the following table were prepared as described in Preparation 149 using the appropriate aryl bromide. Differing methods or gradients can be used to work up or purify the compounds, which would be apparent to one skilled in the art.

TABLE 10

| Prep | Chemical Name | Structure | ES-MS m/z |
|---|---|---|---|
| 150 | Methyl (1S,2S)-2-(4-(2- (trifluoromethyl)imidazo[1,2- a]pyrimidin-6- yl)phenyl)cyclopropane-1- carboxylate | 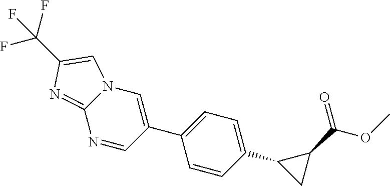 [SMILES: COC(=O)[C@H]1C[C@@H]1c1ccc(-c2cnc3nc(C(F)(F)F)cn3c2)cc1] | 362.2 (M+H) |
| 151 | Methyl (1S,2S)-2-(4- (imidazo[1,2-a]pyrimidin-6- yl)phenyl)cyclopropane-1- carboxylate | 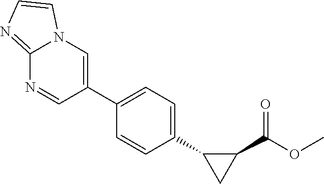 [SMILES: COC(=O)[C@H]1C[C@@H]1c1ccc(-c2cnc3nccn3c2)cc1] | 294.2 (M + H) |

### Preparation 152

### 5-(4-Bromo-3-fluorophenyl)-2-methyl-2H-pyrazolo[3,4-b]pyridine

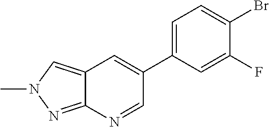 [SMILES: Cn1cc2cc(-c3ccc(Br)c(F)c3)cnc2n1]

A suspension of (2-methyl-2H-pyrazolo[3,4-b]pyridin-5-yl) boronic acid (1.00 g, 5.65 mmol), Pd(dppf)Cl₂·CH₂Cl₂ (923 mg, 1.13 mmol), 1-bromo-2-fluoro-4-iodobenzene (3.40 g, 11.3 mmol), and Cs₂CO₃ (7.36 g, 22.6 mmol) in 3:1 1,4-dioxane/water (25 mL) was sparged with N₂ for 5 min. The resulting mixture was stirred at 60° C. for 2 h, then cooled and diluted with EtOAc and water. The layers were separated, and the aq. layer was extracted with EtOAc (3×). The organic layers were combined, washed with sat. aq. NaCl, dried over Na₂SO₄, filtered, and concentrated under reduced pressure. The residue was purified by silica gel chromatography using a gradient of 0-2% MeOH in DCM to obtain the title compound (1.64 g, 82%, 86% purity) as a yellow foam. ES-MS m/z 306,308 (M+H).

The compounds in the following table were prepared as described in Preparation 152 from the appropriate aryl boronic acid and appropriate aryl halide. Differing methods can be used to work up or purify the compounds, which would be apparent to one skilled in the art.

TABLE 11

| Prep | Chemical Name | Structure | ES-MS m/z |
|---|---|---|---|
| 153ª | 6-(4-Bromo-3- (trifluoromethyl)phenyl)imidazo[1,2- a]pyrimidine | 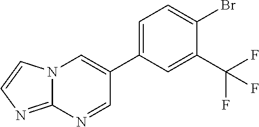 [SMILES: FC(F)(F)c1cc(-c2cnc3nccn3c2)ccc1Br] | 342, 344 (M + H) |
| 154^{b} | 5-(5-Chloro-6- (trifluoromethyl)pyridin-2-yl)-2- methyl-2H-pyrazolo[3,4-b]pyridine | 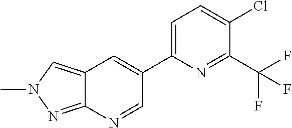 [SMILES: Cn1cc2cc(-c3ccc(Cl)c(C(F)(F)F)n3)cnc2n1] | 313 (M + H) |

^{a}Used 1-bromo-4-iodo-2-(trifluoromethyl)benzene, 90° C. for 1 h.
^{b}Used 6-bromo-3-chloro-2-(trifluoromethyl)pyridine, 90° C. for 1 h, precipitated with EtOAc and collected solids.

### Preparation 155

### Potassium ((1S,2S)-2-(ethoxycarbonyl)cyclopropyl)trifluoroboratecarboxylate

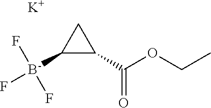

The synthesis and chiral analysis for the title compound was conducted in a manner similar as described in Thach Nguyen, Sanil Sreekumar, Shuai Wang, Qi Jiang, Florian Montel, and Frederic Buono, Organic Process Research & Development 2022 26 (10), 2979-2985. A solution of potassium vinyltrifluoroborate (250 g, 1.87 mol) and Ru-(R)-Pheox (CAS #1421679-43-7, 11.9 g, 18.7 mmol) in EtOAc (2.25 L) was treated with a solution of ethyl diazoacetate (426 g, 3.73 mol) in EtOAc (500 mL) dropwise at 0-5° C. under N₂ over 4 h. The resulting mixture was warmed to 25° C. and stirred for 5 h. MTBE (4.12 L) was added, and the resulting brown slurry was cooled to 0-5° C. and agitated for 30 min. The reaction was repeated 4 times on the same scale, and all 5 reactions were combined for filtration. The combined filter cake was washed with 2:3 EtOAc/MTBE (5 L) and dried under reduced pressure at 50° C. for 5 h to obtain the title compound (920 g, 45%) as white solid. ES-MS m/z 181.1 (M+). [a]_{D}²⁰=+13.42° (C=1.0, ACN).

### Preparation 156

### Ethyl (1S,2S)-2-(2-fluoro-4-(2-methyl-2H-pyrazolo[3,4-b]pyridin-5-yl)phenyl)cyclopropane-1-carboxylate

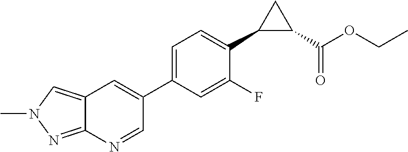 [SMILES: CCOC(=O)[C@H]1C[C@@H]1c1ccc(-c2cnc3nn(C)cc3c2)cc1F]

A mixture of cataCXium Pd G4 (CAS #2230788-67-5, 255 mg, 0.34 mmol), 5-(4-bromo-3-fluorophenyl)-2-methyl-2H-pyrazolo[3,4-b]pyridine (1.05 g, 3.4 mmol), ethyl (1S,2S)-2-(trifluoro-14-boraneyl)cyclopropane-1-carboxylate, potassium salt (906 mg, 4.1 mmol), Cs₂CO₃ (0.823 mL, 10.3 mmol) in toluene (15 mL) was degassed with N₂, then water (1.5 mL) was added, and the flask was evacuated and backfilled with N₂ three times. The reaction vessel was heated to 80° C. for 16 h, then cooled, diluted with EtOAc, dried over Na₂SO₄, filtered over diatomaceous earth, and concentrated. The residue was purified by silica gel chromatography using a gradient of 1-2% MeOH in DCM, followed by reverse phase flash chromatography (150 g C18 column, 110 mL/min) using a gradient of 35-60% ACN/10 mM aq. NH₄HCO₃ (with 5% MeOH) to obtain the title compound (600 mg, 52%) as an off-white solid. ES-MS m/z 340.2 (M+H).

The compounds in the following table were prepared in a similar manner as described in Preparation 156 from the appropriate aryl halide from Table 11 and either potassium ((1S,2S)-2-(ethoxycarbonyl)cyclopropyl)trifluoroborate or commercial potassium rac-((trans)-2-(ethoxycarbonyl)cyclopropyl)trifluoroborate. Differing methods can be used to work up or purify the compounds, which would be apparent to one skilled in the art.

TABLE 12

| Prep | Chemical Name | Structure | ES-MS m/z |
|---|---|---|---|
| 157ª | Ethyl (1S,2S)-2-(6-(2-methyl-2H- pyrazolo[3,4-b]pyridin-5-yl)-2- (trifluoromethyl)pyridin-3- yl)cyclopropane-1-carboxylate | 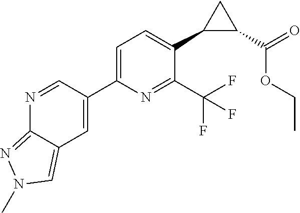 [SMILES: CCOC(=O)[C@H]1C[C@@H]1c1ccc(-c2cnc3nn(C)cc3c2)nc1C(F)(F)F] | 391 (M + H) |
| 158^{b} | rac-Ethyl (trans)-2-(4- (imidazo[1,2-a]pyrimidin-6-yl)- 2- (trifluoromethyl)phenyl)cyclopro- pane-1-carboxylate | 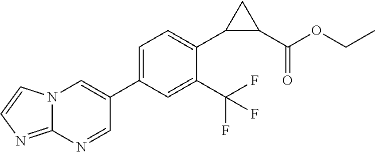 [SMILES: CCOC(=O)C1CC1c1ccc(-c2cnc3nccn3c2)cc1C(F)(F)F] | 376 (M + H) |
| 159^{c} | rac-Ethyl (trans)-2-(4-amino-3- methylphenyl)cyclopropane-1- carboxylate | 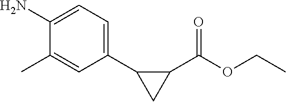 [SMILES: CCOC(=O)C1CC1c1ccc(N)c(C)c1] | 220 (M + H) |

^{a}filtered, and the concentrated filtrate was used in the next step without purification.
^{b}purified by silica gel chromatography, 0-100% 3:1 EtOH:EtOAc in heptane.
^{c}heated at 110° C., purified by silica gel chromatography using a gradient of 0-75% EtOAc in heptane.

### Preparation 160

### rac-Ethyl (trans)-2-(4-(3-(4-fluorophenyl) ureido)-3-methylphenyl)cyclopropane-1-carboxylate

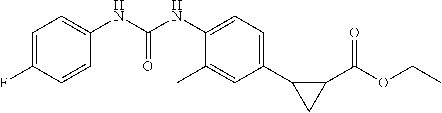 [SMILES: CCOC(=O)C1CC1c1ccc(NC(=O)Nc2ccc(F)cc2)c(C)c1]

A mixture of rac-ethyl (trans)-2-(4-amino-3-methylphenyl)cyclopropane-1-carboxylate (76.0 mg, 347 μmol) in DCM (2 mL) was treated with TEA (106 μL, 762 μmol) and 4-fluorophenyl isocyanate (47.2 μL, 416 μmol). The resulting mixture was stirred at RT for 16 h then diluted with 0.2 M aq. HCl (10 mL) and EtOAc (10 mL). The aq. layer was extracted with EtOAc (2× 8 mL). The organic layers were combined, washed with sat. aq. NaCl (5 mL), dried over Na₂SO₄, filtered, and concentrated under reduced pressure to obtain a quantitative yield of the title compound. ES-MS m/z 329.2 m/z (M+H).

### Preparation 161

### Methyl (1S,2S)-2-(4-((tert-butoxycarbonyl)amino)phenyl)cyclopropane-1-carboxylate

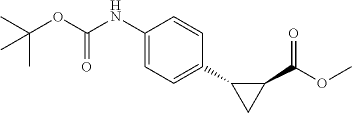 [SMILES: COC(=O)[C@H]1C[C@@H]1c1ccc(NC(=O)OC(C)(C)C)cc1]

A solution of methyl (1S,2S)-2-(4-bromophenyl)cyclopropane-1-carboxylate (6.5 g, 25 mmol) in 1,4-dioxane (8 mL) was added tert-butyl carbamate (4.5 g, 38 mmol), Pd₂(dba)₃ (2.4 g, 2.5 mmol), XPhos (1.2 g, 2.5 mmol) and Cs₂CO₃ (13 g, 38 mmol). The mixture was vacuum degassed and purged with N₂ (3 x) then stirred at 100° C. under N₂ for 16 h. The reaction mixture was cooled and filtered. The filtrate was diluted with water (150 mL) and extracted with EtOAc (3× 150 mL). The organic layers were combined, washed with sat. aq. NaCl (150 mL), dried over Na₂SO₄, and concentrated under reduced pressure. The residue was purified by silica gel chromatography using a gradient of 0-10% EtOAc in PE, followed by a second purification on silica using a gradient of 0-100% DCM in PE to obtain the title compound (4.5 g, 60%) as a yellow solid. ES-MS m/z 236.1 (M-tBu+H).

### Preparation 162

### Methyl (1S,2S)-2-(4-aminophenyl)cyclopropane-1-carboxylate hydrochloride

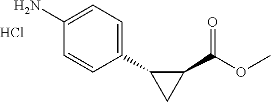

A 0° C. solution of methyl (1S,2S)-2-(4-((tert-butoxycarbonyl)amino)phenyl)cyclopropane-1-carboxylate (2.2 g, 7.5 mmol) in MeOH (20 mL) was treated with 2 M HCl in MeOH (16 mL, 32 mmol) dropwise over 10 min. The resulting mixture was stirred at RT for 2 h before concentrating under reduced pressure to obtain the title compound (1.5 g, 79%, 90% purity) as a yellow solid. ES-MS m/z 191.9 (M+H).

### Preparation 163

### Methyl (1S,2S)-2-(4-(3-(4-fluorophenyl)ureido)phenyl)cyclopropane-1-carboxylate

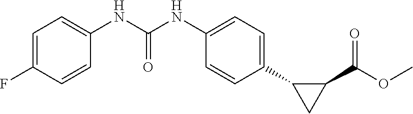 [SMILES: COC(=O)[C@H]1C[C@@H]1c1ccc(NC(=O)Nc2ccc(F)cc2)cc1]

The title compound was prepared as described in Preparation 160 using methyl (1S,2S)-2-(4-aminophenyl)cyclopropane-1-carboxylate hydrochloride and purifying by reverse phase prep-HPLC (XSelect® CSH C18, 30×150 mm, 5 μm, 70 mL/min) eluting with 30-70% ACN/10 mM aq. NH₄HCO₃, pH10 (with 5% MeOH). ES-MS m/z 329.2 (M+H).

### Preparation 164

### (1S,2S)-2-(4-(Imidazo[1,2-a]pyrimidin-6-yl)phenyl)cyclopropane-1-carboxylic acid

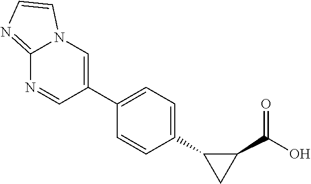 [SMILES: O=C(O)[C@H]1C[C@@H]1c1ccc(-c2cnc3nccn3c2)cc1]

A mixture of methyl (1S,2S)-2-(4-(imidazo[1,2-a]pyrimidin-6-yl)phenyl)cyclopropane-1-carboxylate (3.5 g, 11 mmol) in ACN (35 mL) and water (35 mL) was treated with 1,5,7-triazabicyclo[4.4.0]dec-5-ene (TBD, 3.2 g, 23 mmol). The mixture was stirred at 25° C. for 12 h. The reaction mixture was adjusted to pH7 with 1N aq. HCl. The resultant precipitate was collected by suction filtration, washed with water (10 ml), and dried under reduced pressure to obtain the title compound (2.4 g, 72%) as a grey solid. ES-MS m/z 280.0 (M+H).

### Preparation 165

### (1S,2S)-2-(4-(2-Methyl-2H-pyrazolo[3,4-b]pyridin-5-yl)phenyl)cyclopropane-1-carboxylic acid

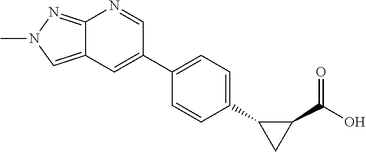 [SMILES: Cn1cc2cc(-c3ccc([C@H]4C[C@@H]4C(=O)O)cc3)cnc2n1]

A solution of methyl (1S,2S)-2-(4-(2-methyl-2H-pyrazolo[3,4-b]pyridin-5-yl)phenyl)cyclopropane-1-carboxylate (2.10 g, 6.83 mmol) in THF (34 mL) was treated with water (4.9 mL) and 2M aq. LiOH (10.2 mL, 20.5 mmol) and stirred at 40° C. for 3 h. The reaction mixture was cooled to RT, and 1M aq. HCl (30.7 mL, 30.7 mmol) was added. The mixture concentrated under a stream of N₂ to remove organics and filtered, rinsing with copious amounts of water. The resultant tan solid was freeze-dried to obtain the title compound (1.73 g, 86%). ES-MS m/z 294.2 (M+H).

The compounds in the following table were prepared as described in Preparation 165. Alternatively, after acidification of the reaction mixture, the products can be directly filtered or extracted with EtOAc. These compounds can also be isolated without acidifying as their corresponding Li carboxylate salts.

TABLE 13

| | | | ES-MS |
|---|---|---|---|
| Prep | Chemical Name | Structure | m/z |
| 166ª | (1S,2S)-2-(4-(2-(Trifluoro- methyl)imidazo[1,2-a] pyrimidin-6-yl)phenyl)- cyclopropane-1-carboxylic acid | 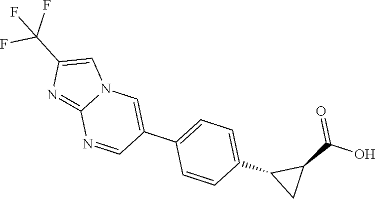 [SMILES: O=C(O)[C@H]1C[C@@H]1c1ccc(-c2cnc3nc(C(F)(F)F)cn3c2)cc1] | 348.2 (M + H) |
| 167^{b} | Lithium (1S,2S)-2-(4- (imidazo[1,2-a]pyrimidin- 6-yl)phenyl)cyclopropane- 1-carboxylate | 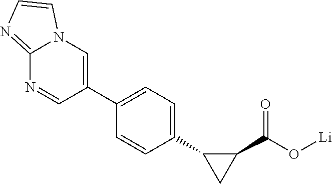 | 280.2 (M + H) |
| 168^{c} | (1S,2S)-2-(2-Fluoro-4-(2- methyl-2H-pyrazolo[3,4-b] pyridin-5-yl)phenyl)cyclo- propane-1-carboxylic acid | 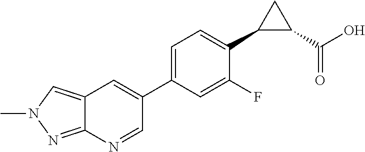 [SMILES: Cn1cc2cc(-c3ccc([C@H]4C[C@@H]4C(=O)O)c(F)c3)cnc2n1] | 311.8 (M + H) |
| 169^{d} | rac-(trans)-2-(4-(Imidazo [1,2-a]pyrimidin-6-yl)-2- (trifluoromethyl)phenyl)- cyclopropane-1-carboxylic acid | 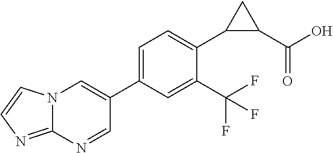 [SMILES: O=C(O)C1CC1c1ccc(-c2cnc3nccn3c2)cc1C(F)(F)F] | 348.0 (M + H) |
| 170^{e} | (1S,2S)-2-(6-(2-Methyl- 2H-pyrazolo[3,4-b]pyridin- 5-yl)-2-(trifluoromethyl)- pyridin-3-yl)cyclopropane- 1-carboxylic acid | 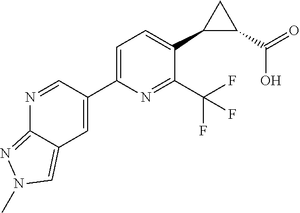 [SMILES: Cn1cc2cc(-c3ccc([C@H]4C[C@@H]4C(=O)O)c(C(F)(F)F)n3)cnc2n1] | 363.2 (M + H) |
| 171^{f} | (1S,2S)-2-(4-((tertbutoxy- carbonyl)amino)phenyl)- cyclopropane-1-carboxylic acid | 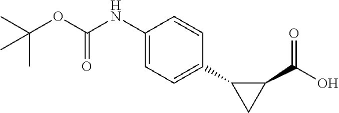 [SMILES: CC(C)(C)OC(=O)Nc1ccc([C@H]2C[C@@H]2C(=O)O)cc1] | 221.9 (M − tBu + H) |
| 172^{d} | (1S,2S)-2-(4-(3-(4-Fluoro- phenyl)ureido)phenyl)cyclo- propane-1-carboxylic acid | 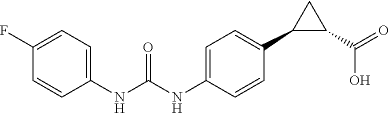 [SMILES: O=C(Nc1ccc(F)cc1)Nc1ccc([C@H]2C[C@@H]2C(=O)O)cc1] | 315.2 (M + H) |
| 173^{g} | rac-(trans)-2-(4-(3-(4- Fluorophenyl)ureido)-3- methylphenyl)cyclopro- pane-1-carboxylic acid | 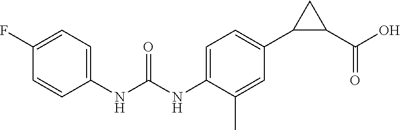 [SMILES: Cc1cc(C2CC2C(=O)O)ccc1NC(=O)Nc1ccc(F)cc1] | 329.0 (M + H) |

^{a.}RT, 24 h.
^{b.}1.25 eq 2M aq. LiOH, 50° C., 20 h, lyophilized to give the Li salt.
^{c.}40° C., 24 h.
^{d.}12 eq 2M aq. LiOH, RT, overnight in 1:1 THF:MeOH, acidified with 12 eq 4M HCl/dioxane and concentrated.
^{e.}13 eq 2M aq. LiOH, 45° C., overnight in 1:1 THF:MeOH, acidified with 13 eq 4M HCl/dioxane, extracted with 4:1 CHCl₃:IPA.
^{f.}2:1 THF:MeOH, RT, 4 h, then acidified with 1M aq. HCl, extracted with EtOAc.
^{g.}6 eq LiOH, 7 eq KOH, 75° C., 4 d, then acidified with 1M aq. HCl, extracted with EtOAc.

### Preparation 174

### (1S,2S)-2-(4-(3,3-Difluorocyclobutane-1-carboxamido)phenyl)cyclopropane-1-carboxylic acid

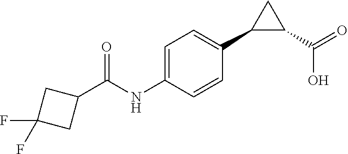 [SMILES: O=C(Nc1ccc([C@H]2C[C@@H]2C(=O)O)cc1)C1CC(F)(F)C1]

A mixture of 3,3-difluorocyclobutane-1-carboxamide (4.48 g, 33.2 mmol), (1S,2S)-2-(4-bromophenyl)cyclopropane-1-carboxylic acid (prepared as described in US 2011/0201623 A1; 4.00 g, 16.6 mmol), XantPhos Pd G4 (CAS #1621274-19-8, 798 mg, 0.830 mmol), and Cs₂CO₃ (16.2 g, 49.8 mmol). The reaction flask was purged with N₂ (×2), and NMP (33.2 mL) was added. The reaction mixture was sparged with N₂ for 10 min, warmed to 45° C. briefly, and then stirred at 80° C. for 20 h. The reaction mixture was cooled to RT, diluted with water (300 mL) and extracted with EtOAc (2×100 mL). The aq. layer was acidified to pH2 with 1M aq. HCl (100 mL) and extracted with EtOAc (4×100 mL). The organic layers were combined and concentrated under reduced pressure. The residue was purified by silica gel chromatography using a gradient of 10-90% (10% EtOH/EtOAc) in heptanes (with 0.05% HOAc) to obtain the title compound (2.14 g, 44%) as a white solid. ES-MS m/z 296.2 (M+H).

### Preparation 175

### (1S,2S)-2-(4-(4,4-Difluorocyclohexane-1-carboxamido)phenyl)cyclopropane-1-carboxylic acid

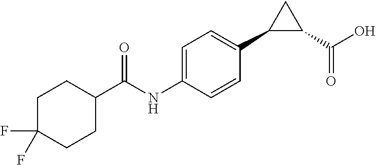 [SMILES: O=C(Nc1ccc([C@H]2C[C@@H]2C(=O)O)cc1)C1CCC(F)(F)CC1]

A mixture of 4,4-difluorocyclohexane-1-carboxamide (2.88 g, 17.6 mmol), XantPhos-Pd-G4 (CAS #1621274-19-8, 736 mg, 0.764 mmol), and Cs₂CO₃ (11.5 g, 35.3 mmol) was purged with N₂. NMP (23.5 mL) and methyl (1S,2S)-2-(4-bromophenyl)cyclopropane-1-carboxylate (3.00 g, 11.8 mmol) were added, and the resulting mixture was sparged with N₂ for 10 min, warmed to 50° C. briefly, and then stirred at 83° C. for 40 h. The reaction mixture was cooled to RT and diluted with water (350 mL) and EtOAc (70 mL). The layers were separated, and the aq. layer was extracted with EtOAc (3×40 mL). The organic layers were combined, dried over Na₂SO₄, filtered, and concentrated under reduced pressure to give methyl (1S,2S)-2-(4-(4,4-difluorocyclohexane-1-carboxamido)phenyl)cyclopropane-1-carboxylate. ES-MS m z 338.2 (M+H). The residue was suspended in THF (40 mL), water (10 mL) and 2M aq. LiOH (17.6 mL), and the resulting mixture was stirred at 50° C. for 16 h. The reaction mixture was cooled to RT, concentrated under reduced pressure, and diluted with EtOAc (100 mL), 1.0M aq. HCl (100 mL) and water (50 mL). The layers were separated, and the aq. layer was extracted with EtOAc (3×75 mL). The organic layers were combined, dried over Na₂SO₄, filtered, and concentrated under reduced pressure. The residue was purified by silica gel chromatography using a gradient of 10-90% (10% EtOH/EtOAc) in heptanes (with 0.05% HOAc) to obtain the title compound (1.83 g, 48%) as an off-white solid. ES-MS m/z 324.2 (M+H).

### Preparation 176

### (1S,2S)-2-(4-(4,4,5,5-Tetramethyl-1,3,2-dioxaborolan-2-yl)phenyl)cyclopropane-1-carboxylic acid

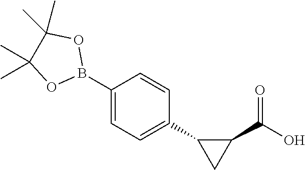 [SMILES: CC1(C)OB(c2ccc([C@H]3C[C@@H]3C(=O)O)cc2)OC1(C)C]

A mixture of methyl (1S,2S)-2-(4-(4,4,5,5-tetramethyl-1,3,2-dioxaborolan-2-yl)phenyl)cyclopropane-1-carboxylate (1.00 g, 3.31 mmol) and trimethylstannanol (1.80 g, 9.93 mmol) in DCE (10 mL) was stirred at 80° C. overnight then concentrated under reduced pressure. The residue was partitioned between EtOAc and water, and the organic layer was washed with 1N aq. HCl, dried over MgSO₄, filtered, and concentrated under reduced pressure to obtain a quantitative yield of the title compound. ES-MS m/z 287.2 (M−H).

### Preparation 177

### tert-Butyl 2-(4-bromophenyl)-2-methylcyclopropane-1-carboxylate (mixture of cis/trans enantiomeric pairs)

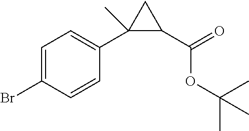 [SMILES: CC(C)(C)OC(=O)C1CC1(C)c1ccc(Br)cc1]

A mixture of 1-bromo-4-(prop-1-en-2-yl)benzene (1.0 g, 5.1 mmol) and rhodium (II) acetate (0.22 g, 0.51 mmol) in THF (20 mL) was treated with tert-butyl 2-diazoacetate (0.87 g, 6.1 mmol). The resulting mixture was stirred at 18° C. for 16 h, then concentrated under reduced pressure. The residue was purified by silica gel chromatography using a gradient of 0-8% EtOAc in PE to obtain the title compound (1.6 g, 19%, 75% purity) as a white solid. ES-MS m/z 255,257 (M-t-Bu+H).

### Preparation 178

### tert-Butyl 2-methyl-2-(4-(2-methyl-2H-pyrazolo[3,4-b]pyridin-5-yl)phenyl)cyclopropane-1-carboxylate (Mixture of Cis/Trans Enantiomeric Pairs)

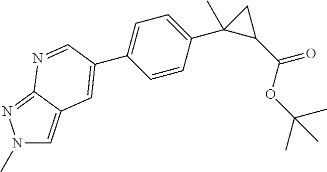 [SMILES: Cn1cc2cc(-c3ccc(C4(C)CC4C(=O)OC(C)(C)C)cc3)cnc2n1]

To a mixture of tert-butyl-2-(4-bromophenyl)-2-methylcyclopropane-1-carboxylate (mixture of cis/trans enantiomeric pairs, 395 mg, 952 μmol, 75% purity) and (2-methyl-2H-pyrazolo[3,4-b]pyridin-5-yl) boronic acid (168 mg, 952 μmol) in 1,4-dioxane (6 mL) and water (0.6 mL) was added Na₂CO₃ (0.14 mL, 1.90 mmol) and Pd(dppf)Cl₂ (104 mg, 143 μmol). After stirring at 90° C. for 16 h under N₂, the reaction mixture was cooled, diluted with EtOAc (8 mL) and filtered. The filtrate was concentrated under reduced pressure and the residue was purified by silica gel chromatography using a gradient of 0-6% MeOH in DCM to obtain the title compound (371 mg, 95%, 89% purity) as a brown oil. ES-MS m/z 364.3 (M+H).

### Preparation 179

### 2-Methyl-2-(4-(2-methyl-2H-pyrazolo[3,4-b]pyridin-5-yl)phenyl)cyclopropane-1-carboxylic acid (mixture of cis/trans enantiomeric pairs)

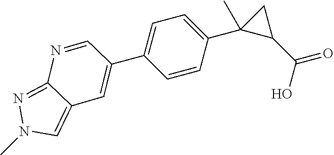 [SMILES: Cn1cc2cc(-c3ccc(C4(C)CC4C(=O)O)cc3)cnc2n1]

A solution of tert-butyl 2-methyl-2-(4-(2-methyl-2H-pyrazolo[3,4-b]pyridin-5-yl)phenyl)cyclopropane-1-carboxylate (mixture of cis/trans enantiomeric pairs, 365 mg, 894 μmol, 89% purity) in DCM (3 mL) was treated with TFA (1.00 mL, 13.0 mmol), and the resulting mixture was stirred at 25° C. for 3 h. The reaction mixture was concentrated under reduced pressure to obtain the title compound (278 mg, 91%, 90% purity) as a brown oil. ES-MS m/z 308.2 (M+H).

### Preparation 180

### Methyl (R)-2-amino-3-(1-methyl-1H-pyrazol-4-yl)propanoate hydrochloride

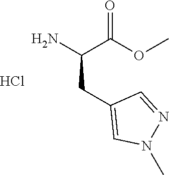

A solution of (R)-2-(((benzyloxy)carbonyl)amino)-3-(1-methyl-1H-pyrazol-4-yl)propanoic acid (180 g, 593 mmol) in MeOH (1.26 L) was treated with SOCl₂ (141 g, 1.19 mol), and the resulting mixture was stirred at 70° C. for 1 h. The reaction mixture was filtered, and the filtrate was concentrated under reduced pressure. The residue was dissolved in MeOH (1.0 L) and Pd/C (50 wt %, 100 g) was added. The suspension was degassed and purged with H₂ (3×) and stirred under H₂ (0.14 MPa) at 25° C. for 12 h. The reaction mixture was filtered, and the filtrate was concentrated under reduced pressure to obtain the title compound (130 g, 87%) as a white solid. ES-MS m/z 184.1 (M+H).

### Preparation 181

### Methyl (R)-2-((1S,2S)-2-(4-bromophenyl)cyclopropane-1-carboxamido)-3-(1-methyl-1H-pyrazol-4-yl)propanoate

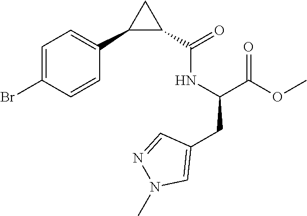 [SMILES: COC(=O)[C@@H](Cc1cnn(C)c1)NC(=O)[C@H]1C[C@@H]1c1ccc(Br)cc1]

To a mixture of HATU (5.13 g, 13.5 mmol) and (1S,2S)-2-(4-bromophenyl)cyclopropane-1-carboxylic acid (prepared as described in US 2011/0201623 A1; 2.50 g, 10.4 mmol) under N₂ was added DMF (40 mL), methyl (R)-2-amino-3-(1-methyl-1H-pyrazol-4-yl)propanoate HCl (2.9 g, 11.4 mmol), and DIEA (11 mL, 62 mmol). The resulting mixture was stirred at RT for 15 h, then poured into sat. aq. NaHCO₃ (100 mL) and EtOAc (50 ml). The aq. layer was extracted with EtOAc (3×50 mL). The organic layers were combined, washed with water (2×30 mL) then sat. aq. NaCl (30 mL), dried over Na₂SO₄, filtered, and concentrated under reduced pressure. The residue was purified by silica gel chromatography using a gradient of 0-5% MeOH in DCM to obtain a quantitative yield of the title compound (5.0 g, 76% purity) as a white solid. ES-MS m/z 406,408 (M+H).

### Preparation 182

### (R)-3-(1-Methyl-1H-pyrazol-4-yl)-2-((1S,2S)-2-(4-(2-methyl-2H-pyrazolo[3,4-b]pyridin-5-yl)phenyl)cyclopropane-1-carboxamido)propanoic acid

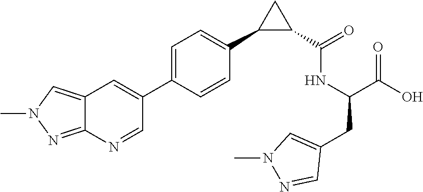 [SMILES: Cn1cc(C[C@@H](NC(=O)[C@H]2C[C@@H]2c2ccc(-c3cnc4nn(C)cc4c3)cc2)C(=O)O)cn1]

A mixture of 1,4-dioxane (70 mL) and water (23 mL) was sparged with N₂ for 10 min then added to a mixture of methyl (R)-2-((1S,2S)-2-(4-bromophenyl)cyclopropane-1-carboxamido)-3-(1-methyl-1H-pyrazol-4-yl)propanoate (3.80 g, 9.35 mmol), (2-methyl-2H-pyrazolo[3,4-b]pyridin-5-yl) boronic acid (2.65 g, 15.0 mmol), Pd(dppf)Cl₂·CH₂Cl₂ (764 mg, 935 μmol), and Cs₂CO₃ (9.75 g, 29.9 mmol) under N₂. The resulting mixture was stirred at 90° C. for 27 h, then cooled to RT, acidified with 1M aq. HCl to ˜pH3, and concentrated under reduced pressure. The residue was azeotroped with toluene then purified by silica gel chromatography using a gradient of 0-38% (20% MeOH/DCM with 2% HOAc) in DCM. Repeated azeotroping with MeOH/toluene gave the title compound (1.9 g, 45%) as a tan solid. ES-MS m/z 445.2 (M+H).

### Preparation 183

### 1-(4-Bromobenzyl)tetrahydro-1H-thiophen-1-ium bromide

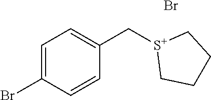 [SMILES: Br ; Brc1ccc(C[S+]2CCCC2)cc1]

A mixture of p-bromobenzyl bromide (200 g, 784 mmol), acetone (2 L), and tetrahydrothiophene (104 g, 1.18 mol) was stirred at 25° C. for 32 h. The resulting precipitate was filtered, rinsed with acetone (2×1 L) and triturated by stirring with acetone (1 L) at 25° C. for 16 h. Filtered to obtain the title compound (182 g, 66%) as a white solid. ES-MS m/z 257,259 (M⁺).

### Preparation 184

### rac-(trans)-1-(4-Bromophenyl)-5-oxaspiro[2.4]heptan-4-one

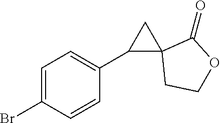 [SMILES: O=C1OCCC12CC2c1ccc(Br)cc1]

To a mixture of 3-methylenedihydrofuran-2(3H)-one (30.0 g, 291 mmol), DCM (600 mL), and 1-(4-bromobenzyl)tetrahydro-1H-thiophen-1-ium bromide (102 g, 291 mmol) at 0° C., was added 1M lithium bis(trimethylsilyl) amide in THF (262 mL, 262 mmol) dropwise (until the mixture cleared) over 30 min. The resulting mixture was stirred for 2 h at 35° C. (internal temperature), then cooled and treated with sat. aq. NH₄Cl (60 mL). Water (300 mL) was added, and the layers were separated. The aq. layer was extracted with DCM (3×300 mL). The organic layers were combined, washed with sat. aq. NaCl (300 mL), dried over Na₂SO₄, filtered, and concentrated under reduced pressure. The residue was triturated by stirring with MTBE (152 mL) at 25° C. for 8 h. Filtration gave the title compound (63 g, 79%) as a white solid. ES-MS m z 267,269 (M+H). Analytical SFC 1:1 mixture, Rt=2.10, 2.29 min (Chiralpak® AD-3, 4.6×150 mm, 3 μm, column temperature 35° C., 220 nm, flow rate 2.5 mL/min, isocratic 20% EtOH (with 0.2% 7M NH₃ in MeOH): 80% CO₂; Waters® ACQUITY UPC2® with PDA).

### Preparation 185

### rac-(trans)-2-(4-Bromophenyl)-1-(2-hydroxyethyl)cyclopropane-1-carboxylic acid

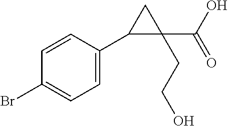 [SMILES: O=C(O)C1(CCO)CC1c1ccc(Br)cc1]

A mixture of rac-(trans)-1-(4-bromophenyl)-5-oxaspiro[2.4]heptan-4-one (45.1 g, 167 mmol) in THF (300 mL) and water (150 mL) was treated with LiOH (12.0 g, 501 mmol) then stirred at 25° C. for 2 h. The reaction mixture was partially concentrated to remove the THF, and 1N HCl was added until pH=3. The resulting suspension was filtered, and the filter cake was washed with water (400 mL) then triturated with water (225 mL) at 25° C. The suspension was filtered, and the filter cake was dried under reduced pressure to obtain the title compound (41 g, 86% yield) as a white solid. ES-MS m/z 285,287 (M+H). Analytical SFC 1:1 mixture, Rt=2.59, 3.36 min (Chiralpak® AD-3, 4.6×150 mm, 3 μm, column temperature 35° C., 220 nm, flow rate 2.5 mL/min, isocratic 20% EtOH (with 0.2% 7M NH₃ in MeOH): 80% CO₂; Waters® ACQUITY UPC²® with PDA).

### Preparation 186

### (S)-1-(Naphthalen-1-yl) ethan-1-amine (1R,2R)-2-(4-bromophenyl)-1-(2-hydroxyethyl)cyclopropane-1-carboxylate

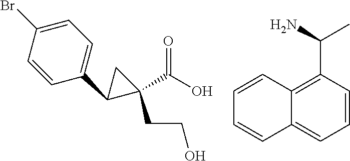

A mixture of rac-(trans)-2-(4-bromophenyl)-1-(2-hydroxyethyl)cyclopropane-1-carboxylic acid (31.0 g, 108 mmol) in methyl acetate (930 mL) was stirred at 25° C. for 10 min. (S)-1-(1-Naphthyl)ethylamine (9.59 mL, 59.5 mmol) was added, and the resulting mixture was stirred for 16 h at 27° C. (internal temperature). The resulting precipitate was filtered, then triturated by stirring with MTBE (210 mL) at 25° C. for 6 h. Filtration gave the title compound (19.7 g, 40%) as a white solid.

A sample of the title compound (5 mg) was dissolved in water (1 mL) and adjusted to pH=3 with 1N aq. HCl. The resulting solid was filtered and used for analytical SFC ˜98% ee, Rt=3.31 min, isomer 2 (Chiralpak® AD-3, 4.6×150 mm, 3 μm, column temperature 35° C., 220 nm, flow rate 2.5 mL/min, isocratic 20% EtOH (with 0.2% 7M NH₃/MeOH): 80% CO₂; Waters® ACQUITY UPC²® with PDA).

### Preparation 187

### (1R,3R)-1-(4-Bromophenyl)-5-oxaspiro[2.4]heptan-4-one

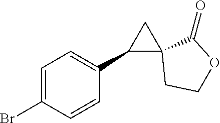 [SMILES: O=C1OCC[C@@]12C[C@@H]2c1ccc(Br)cc1]

A mixture of(S)-1-(naphthalen-1-yl) ethan-1-amine (1R,2R)-2-(4-bromophenyl)-1-(2-hydroxyethyl)cyclopropane-1-carboxylate (80.0 g, 175 mmol) in ACN (400 mL) was treated with 12 M HCl (161 mL) dropwise, and the resulting mixture was stirred at 25° C. for 2 h. Water (1.2 L) was added and stirring was continued at 25° C. for 3 h. The resulting suspension was filtered to obtain the title compound (42.8 g, 91%) as a white solid. ES-MS m/z 267,269 (M+H). Analytical SFC ˜97% ee, Rt=2.12 min, isomer 1 (Chiralpak® AD-3, 4.6×150 mm, 3 μm, column temperature 35° C., 220 nm, flow rate 2.5 mL/min; 20% EtOH (with 0.2% 7M NH₃ in MeOH): 80% CO₂; Waters® ACQUITY UPC²® with PDA).

### Preparation 188

### (1R,3R)-1-(4-(2-methyl-2H-pyrazolo[3,4-b]pyridin-5-yl)phenyl)-5-oxaspiro[2.4]heptan-4-one

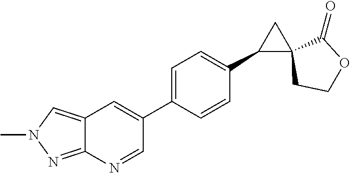 [SMILES: Cn1cc2cc(-c3ccc([C@H]4C[C@@]45CCOC5=O)cc3)cnc2n1]

A mixture of (1R,3R)-1-(4-bromophenyl)-5-oxaspiro[2.4]heptan-4-one (663 mg, 3.74 mmol), Pd(dppf)Cl₂·CH₂Cl₂ (411 mg, 0.562 mmol), Cs₂CO₃ (2.44 g, 7.49 mmol), (2-methyl-2H-pyrazolo[3,4-b]pyridin-5-yl) boronic acid (663 mg, 3.7 mmol) in 1,4-dioxane (34 mL) and water (3.4 mL) was sparged with N₂ for 5 min. After stirring at 80° C. for 1.5 h, the reaction mixture was cooled to RT, diluted with DCM, and washed with water. The organic layer was dried over MgSO₄ and concentrated under reduced pressure. The residue was purified by silica gel chromatography using a gradient of 0-3% MeOH in DCM, then recrystallized from EtOAc to obtain the title compound (855 mg, 72%) as a tan solid. ES-MS m/z 320.0 (M+H).

### Preparation 189

### 1-(4-Bromo-2-fluorobenzyl)tetrahydro-1H-thiophen-1-ium bromide

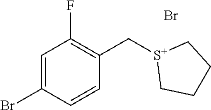 [SMILES: Fc1cc(Br)ccc1C[S+]1CCCC1 ; Br]

A mixture of 4-bromo-1-(bromomethyl)-2-fluorobenzene (20 g, 75 mmol), acetone (200 mL) and tetrahydrothiophene (9.9 g, 0.11 mol) was stirred at RT for 24 h. The resulting precipitate was filtered and rinsed with acetone (200 mL). The collected solids were stirred in acetone (300 mL) at RT for 1 h. The mixture was filtered, and the solids were dried under reduced pressure at RT. All filtrates were combined, concentrated to ˜50 mL, and stirred for 3 days. Acetone (100 mL) was added, and the mixture was stirred for 10 min, then filtered. The collected solids were rinsed with acetone (50 mL) and dried under reduced pressure at RT overnight. The two portions of white solids were combined to obtain the title compound (23.6 g, 89%). ES-MS m/z 275/277 (M⁺).

### Preparation 190

### rac-(trans)-1-(4-Bromo-2-fluorophenyl)-5-oxaspiro[2.4]heptan-4-one

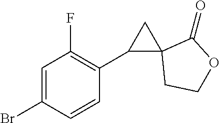 [SMILES: O=C1OCCC12CC2c1ccc(Br)cc1F]

A mixture of 3-methylenedihydrofuran-2(3H)-one (7.7 g, 78 mmol), DCM (148 mL), DMSO (50 mL) and 1-(4-bromo-2-fluorobenzyl)tetrahydro-1H-thiophen-1-ium bromide (18.46 g, 51.84 mmol) under N₂ was stirred at 0° C. and treated with 1M LiHMDS in THF (78 mL, 78 mmol) dropwise, keeping the internal reaction temp below 7° C. After the addition was complete, stirring was continued at 0° C. for 30 min. The cold reaction mixture was poured into a stirring mixture of ice/water (200 mL) and DCM (200 mL). The biphasic mixture was stirred for 1 h, warming to 12° C. in that time. The layers were separated, and the aq. layer was extracted with DCM (200 mL). The organic layers were combined, washed with water (200 mL), then sat. aq. NaCl (200 mL), dried over Na₂SO₄, filtered, and concentrated under reduced pressure. The residue was purified by silica gel chromatography using a gradient of 0-20% EtOAc in cyclohexane to obtain the title compound (8.33 g, 56%) as a white solid. ES-MS m/z 2845,287 (M+H).

### Preparation 191

### (1S,3R)-1-(4-Bromo-2-fluorophenyl)-5-oxaspiro[2.4]heptan-4-one

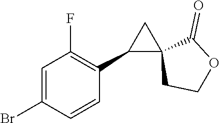 [SMILES: O=C1OCC[C@@]12C[C@@H]2c1ccc(Br)cc1F]

Chiral separation of rac-(trans)-1-(4-bromo-2-fluorophenyl)-5-oxaspiro[2.4]heptan-4-one (3.77 g) was performed by SFC (CHIRALPAK® IG, 30×250 mm, 5 μm, column temp 40° C., flow rate 100 mL/min using a gradient of 20% MeOH in CO₂ for 6.5 min) to obtain the first eluting isomer as the title compound (1.68 g, white solid). 97.8% ee. ES-MS m/z 285,287 (M+H).

### Preparation 192

### (1S,3R)-1-(2-Fluoro-4-(2-methyl-2H-pyrazolo[3,4-b]pyridin-5-yl)phenyl)-5-oxaspiro[2.4]heptan-4-one

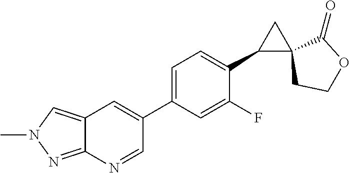 [SMILES: Cn1cc2cc(-c3ccc([C@H]4C[C@@]45CCOC5=O)c(F)c3)cnc2n1]

A mixture of (2-methyl-2H-pyrazolo[3,4-b]pyridin-5-yl) boronic acid (2.22 g, 12.5 mmol), (1S,3R)-1-(4-bromo-2-fluorophenyl)-5-oxaspiro[2.4]heptan-4-one (Isomer 1, 2.38 g, 8.35 mmol) and Cs₂CO₃ (5.44 g, 16.7 mmol), in 1,4-dioxane (75 mL) and water (7.5 mL) was sparged with N₂ for 5 min. Pd(dppf)Cl₂·CH₂Cl₂ (916 mg, 1.25 mmol) was added and the resulting mixture was stirring at 90° C. for 1 h. The reaction mixture was cooled to RT and diluted with water (200 mL) and EtOAc (200 mL). The layers were separated, and the aq. layer was extracted with EtOAc (2×200 mL). The organic layers were combined, filtered through a plug of Na₂SO₄ and concentrated under reduced pressure. The residue was purified by silica gel chromatography using a gradient of 0-50% (10% MeOH/EtOAc) in cyclohexane, then switching to 0-40% (10% MeOH/EtOAc) in EtOAc to obtain the title compound (2.55 g, 91%) as a brown solid. ES-MS m/z 338.0 (M+H).

### Example 1

### (1S,2S)-N—((R)-1-((1-(5-Bromo-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

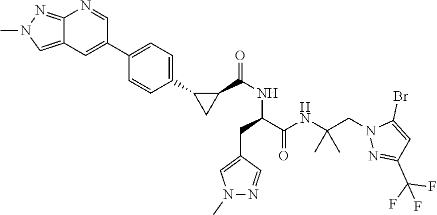 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2Br)cn1]

A mixture of (1S,2S)-2-(4-(2-methyl-2H-pyrazolo[3,4-b]pyridin-5-yl)phenyl)cyclopropane-1-carboxylic acid (805 mg, 2.74 mmol) and (R)-2-amino-N-(1-(5-bromo-3-(trifluoromethyl)-1H-pyrazol-1-yl)-2-methylpropan-2-yl)-3-(1-methyl-1H-pyrazol-4-yl)propanamide (1.20 g, 2.74 mmol) in DMF (5 mL) was treated with DIEA (1.43 mL, 8.23 mmol) and HATU (1.25 g, 3.29 mmol). After 3 h at RT, the reaction mixture was quenched with water and concentrated to dryness. The residue was purified by silica gel chromatography using a gradient of 20-100% (3:1 EtOAc/EtOH) in DCM to obtain the title compound (1.25 g, 64%) as a white solid. ES-MS m/z 712,714 (M+H).

The compounds in the following table were prepared as described in Example 1 using the appropriate carboxylic acid or Li carboxylate salt and the appropriate amine or amine salt. (1S,2S)-2-(4-Bromophenyl)cyclopropane-1-carboxylic acid was prepared as described in US 2011/0201623 A1. Reactants can be added in different orders or in differing equivalency. DMA is a suitable replacement for DMF. Reaction times may vary (30 min to overnight), and differing methods can be used to work up or purify the compounds (normal phase, or high, neutral or low pH reversed phase). Such variances would be apparent to one skilled in the art.

TABLE 14

| Prep/ | | | ES-MS |
|---|---|---|---|
| Example | Chemical Name | Structure | m/z |
| Ex 2 | (1S,2S)-N-((R)-1-((1- (5-Bromo-3-(trifluoro- methyl)-1H-pyrazol-1- yl)-2-methylpropan-2- yl)amino)-3-(imidazo [1,2-a]pyrimidin-3-yl)- 1-oxopropan-2-yl)-2- (4-(2-methyl-2H-pyr- azolo[3,4-b]pyridin-5- yl)phenyl)cyclopro- pane-1-carboxamide | 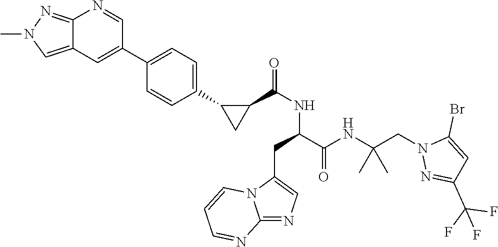 [SMILES: Cn1cc2cc(-c3ccc([C@H]4C[C@@H]4C(=O)N[C@H](Cc4cnc5ncccn45)C(=O)NC(C)(C)Cn4nc(C(F)(F)F)cc4Br)cc3)cnc2n1] | 749, 751 (M + H) |
| Ex 3 | (1S,2S)-N-((R)-1-((1- (5-Bromo-3-(trifluoro- methyl)-1H-pyrazol-1- yl)-2-methylpropan-2- yl)amino)-3-(imidazo [1,2-a]pyrimidin-3-yl)- 1-oxopropan-2-yl)-2- (4-(2-(trifluorometh- yl)imidazo[1,2-a] pyrimidin-6-yl)phen- yl)cyclopropane-1- carboxamide | 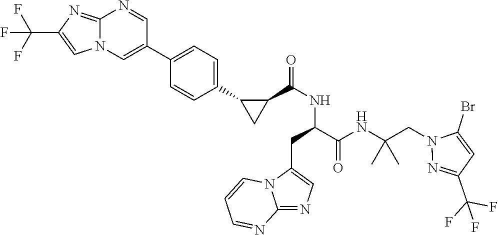 [SMILES: CC(C)(Cn1nc(C(F)(F)F)cc1Br)NC(=O)[C@@H](Cc1cnc2ncccn12)NC(=O)[C@H]1C[C@@H]1c1ccc(-c2cnc3nc(C(F)(F)F)cn3c2)cc1] | 803, 805 (M + H) |
| Ex 4 | (1S,2S)-N-((R)-1-((1- (5-Bromo-3-(trifluoro- methyl)-1H-pyrazol-1- yl)-2-methylpropan-2- yl)amino)-3-(1-meth- yl-1H-pyrazol-4-yl)- 1-oxopropan-2-yl)-N- methyl-2-(4-(2-methyl- 2H-pyrazolo[3,4-b] pyridin-5-yl)phenyl) cyclopropane-1- carboxamide | 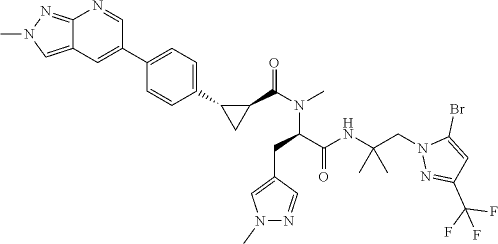 [SMILES: CN(C(=O)[C@H]1C[C@@H]1c1ccc(-c2cnc3nn(C)cc3c2)cc1)[C@H](Cc1cnn(C)c1)C(=O)NC(C)(C)Cn1nc(C(F)(F)F)cc1Br] | 726, 728 (M + H) |
| Ex 5 | (1S,2S)-N-((R)-1-((1- (5-Bromo-3-(trifluoro- methyl)-1H-pyrazol-1- yl)-2-methylpropan-2- yl)amino)-3-(1-methyl- 1H-pyrazol-4-yl)-1- oxopropan-2-yl)-2-(2- fluoro-4-(2-methyl- 2H-pyrazolo[3,4-b] pyridin-5-yl)phenyl)- N-methylcyclopro- pane-1-carboxamide | 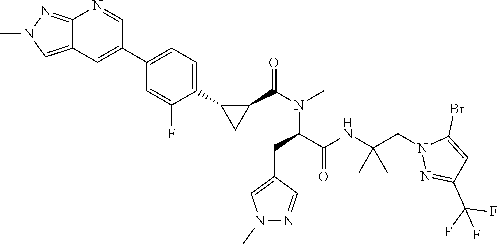 [SMILES: CN(C(=O)[C@H]1C[C@@H]1c1ccc(-c2cnc3nn(C)cc3c2)cc1F)[C@H](Cc1cnn(C)c1)C(=O)NC(C)(C)Cn1nc(C(F)(F)F)cc1Br] | 744, 746 (M + H) |
| Ex 6 | (1S,2S)-2-(2-Fluoro- 4-(2-methyl-2H- pyrazolo[3,4-b] pyridin-5-yl)phenyl)- N-((2R)-1-((2-methyl- 1-(5-(2-(tetrahydro- 2H-pyran-2-yl)-2H- 1,2,3-triazol-4-yl)- 3-(trifluoromethyl)- 1H-pyrazol-1-yl)pro- pan-2-yl)amino)-3-(1- methyl-1H-pyrazol-4- yl)-1-oxopropan-2- yl)cyclopropane-1- carboxamide | 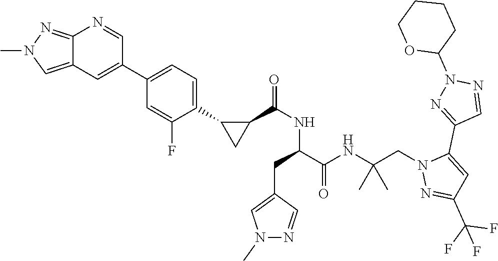 [SMILES: Cn1cc(C[C@@H](NC(=O)[C@H]2C[C@@H]2c2ccc(-c3cnc4nn(C)cc4c3)cc2F)C(=O)NC(C)(C)Cn2nc(C(F)(F)F)cc2-c2cnn(C3CCCCO3)n2)cn1] | 803.6 (M + H) |
| Prep 193 | (1S,2S)-2-(4-Bromo- phenyl)-N-methyl-N- ((R)-1-((2-methyl-1- (5-(1-methyl-6-oxo- 1,6-dihydropyridin-3- yl)-3-(trifluorometh- yl)-1H-pyrazol-1-yl)- propan-2-yl)amino)-3- (1-methyl-1H-pyrazol- 4-yl)-1-oxopropan-2- yl)cyclopropane-1- carboxamide | 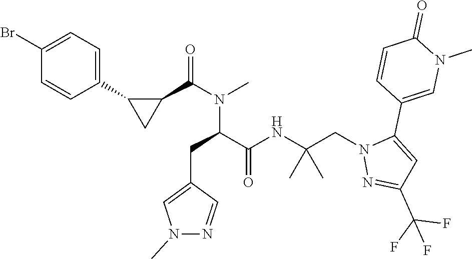 [SMILES: CN(C(=O)[C@H]1C[C@@H]1c1ccc(Br)cc1)[C@H](Cc1cnn(C)c1)C(=O)NC(C)(C)Cn1nc(C(F)(F)F)cc1-c1ccc(=O)n(C)c1] | 702, 704 (M + H) |
| Prep 194 | (1S,2S)-2-(4-Bromo- phenyl)-N-((R)-3- (imidazo[1,2-a]pyr- imidin-3-yl)-1-((2- methyl-1-(4-(trifluoro- methyl)-1H-pyrazol- 1-yl)propan-2-yl)- amino)-1-oxopropan- 2-yl)cyclopropane-1- carboxamide | 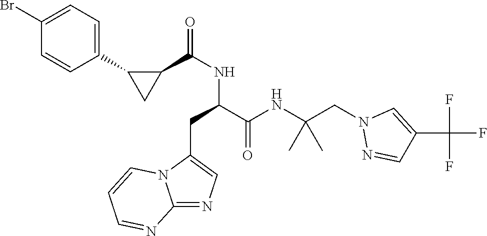 [SMILES: CC(C)(Cn1cc(C(F)(F)F)cn1)NC(=O)[C@@H](Cc1cnc2ncccn12)NC(=O)[C@H]1C[C@@H]1c1ccc(Br)cc1] | 618, 620 (M + H) |
| Prep 195 | (trans)-2-(4-Bromo- 2-fluorophenyl)-N- ((R)-3-(imidazo[1,2- a]pyrimidin-3-yl)-1- ((2-methyl-1-(4- (trifluoromethyl)- 1H-pyrazol-1-yl)- propan-2-yl)amino)- 1-oxopropan-2-yl)- cyclopropane-1- carboxamide, mixture of 2 diastereomers | 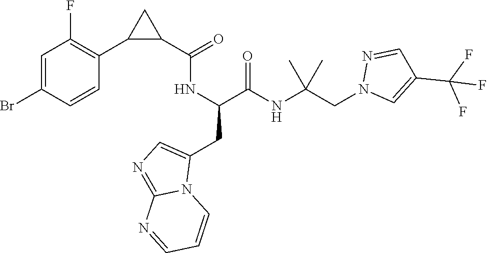 [SMILES: CC(C)(Cn1cc(C(F)(F)F)cn1)NC(=O)[C@@H](Cc1cnc2ncccn12)NC(=O)C1CC1c1ccc(Br)cc1F] | 636, 638 (M + H) |
| Prep 196 | (trans)-2-(5-Bromo- pyridin-2-yl)-N-((R)- 3-(imidazo[1,2-a] pyrimidin-3-yl)-1-((2- methyl-1-(4-(trifluoro- methyl)-1H-pyrazol- 1-yl)propan-2-yl)- amino)-1-oxopropan- 2-yl)cyclopropane-1- carboxamide, mixture of 2 diastereomers | 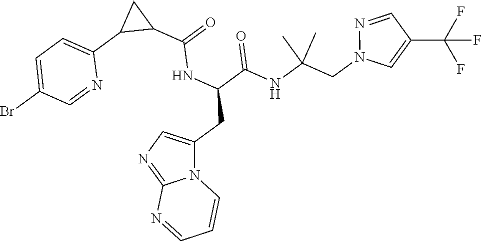 [SMILES: CC(C)(Cn1cc(C(F)(F)F)cn1)NC(=O)[C@@H](Cc1cnc2ncccn12)NC(=O)C1CC1c1ccc(Br)cn1] | 619, 621 (M + H) |
| Prep 197 | (trans)-N-((R)-1-((1- (5-Bromo-3-(trifluoro- methyl)-1H-pyrazol- 1-yl)-2-methylpropan- 2-yl)amino)-3-(1- methyl-1H-pyrazol-4- yl)-1-oxopropan-2-yl)- 2-(5-bromothiophen- 2-yl)cyclopropane-1- carboxamide, mixture of 2 diastereomers | 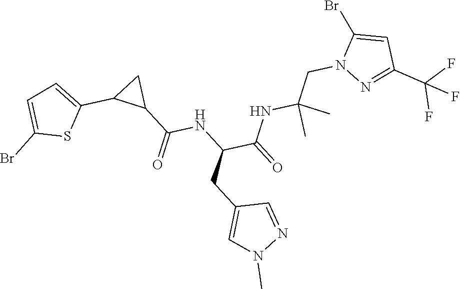 [SMILES: Cn1cc(C[C@@H](NC(=O)C2CC2c2ccc(Br)s2)C(=O)NC(C)(C)Cn2nc(C(F)(F)F)cc2Br)cn1] | 667 (M + H) |
| Prep 198 | (1S,2S)-N-((2R)-1-((2- Methyl-1-(5-(2-(tetra- hydro-2H-pyran-2-yl)- 2H-1,2,3-triazol-4-yl)- 3-(trifluoromethyl)- 1H-pyrazol-1-yl)pro- pan-2-yl)amino)-3-(1- methyl-1H-pyrazol-4- yl)-1-oxopropan-2-yl)- 2-(4-(4,4,5,5-tetra- methyl-1,3,2-dioxa- borolan-2-yl)phenyl)- cyclopropane-1- carboxamide | 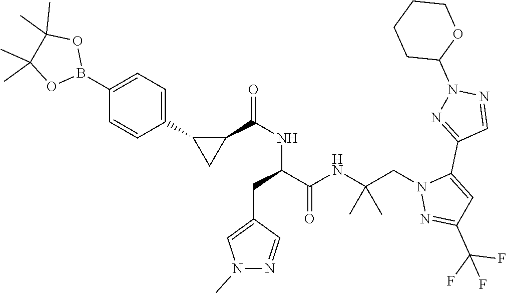 [SMILES: Cn1cc(C[C@@H](NC(=O)[C@H]2C[C@@H]2c2ccc(B3OC(C)(C)C(C)(C)O3)cc2)C(=O)NC(C)(C)Cn2nc(C(F)(F)F)cc2-c2cnn(C3CCCCO3)n2)cn1] | 780.6 (M + H) |
| Prep 199 | tert-Butyl (4-((1S,2S)- 2-(((R)-1-((1-(4- fluorophenyl)-2-meth- ylpropan-2-yl)amino)- 3-(1-methyl-1H-pyr- azol-4-yl)-1-oxopro- pan-2-yl)carbamoyl)- cyclopropyl)phenyl)- carbamate | 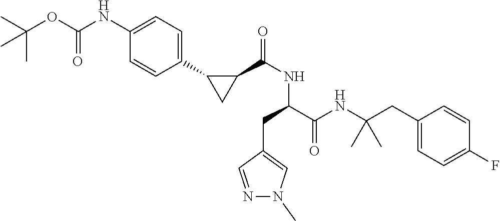 [SMILES: Cn1cc(C[C@@H](NC(=O)[C@H]2C[C@@H]2c2ccc(NC(=O)OC(C)(C)C)cc2)C(=O)NC(C)(C)Cc2ccc(F)cc2)cn1] | 578.4 (M + H) |

### Preparation 200

### (1S,2S)-2-(4-Bromophenyl)-N—((R)-1-((1-(4-fluorophenyl)-2-methylpropan-2-yl)amino)-3-(imidazo[1,2-a]pyrimidin-3-yl)-1-oxopropan-2-yl)cyclopropane-1-carboxamide

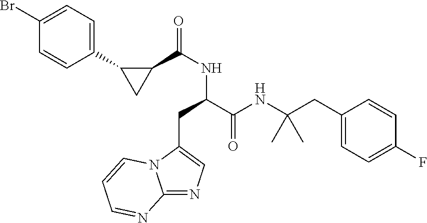 [SMILES: CC(C)(Cc1ccc(F)cc1)NC(=O)[C@@H](Cc1cnc2ncccn12)NC(=O)[C@H]1C[C@@H]1c1ccc(Br)cc1]

A mixture of (1S,2S)-2-(4-bromophenyl)cyclopropane-1-carboxylic acid (prepared as described in US 2011/0201623 A1; 250 mg, 1.0 mmol) and propanephosphonic acid anhydride (50% in EtOAc, 0.76 mL, 1.3 mmol) in THF (7 mL) was treated with DIEA (1.1 mL, 6.2 mmol). After stirring for 1 min, (R)-2-amino-N-(1-(4-fluorophenyl)-2-methylpropan-2-yl)-3-(imidazo[1,2-a]pyrimidin-3-yl)propanamide (531 mg, 1.24 mmol) was added. The reaction mixture was stirred for 2 days, then diluted with sat. aq. NaHCO₃ and EtOAc. The aq. layer was extracted with EtOAc (2×10 mL), and the organic layers were combined, washed with sat. aq. NaCl, dried over Na₂SO₄, filtered, and concentrated under reduced pressure to obtain the title compound (472 mg, 79%) as a yellow foam. ES-MS m/z 578,580 (M+H).

### Preparation 201

### (1S,2S)-N—((R)-3-(Imidazo[1,2-a]pyrimidin-3-yl)-1-((2-methyl-1-(4-(trifluoromethyl)-1H-pyrazol-1-yl)propan-2-yl)amino)-1-oxopropan-2-yl)-2-(4-(4,4,5,5-tetramethyl-1,3,2-dioxaborolan-2-yl)phenyl)cyclopropane-1-carboxamide

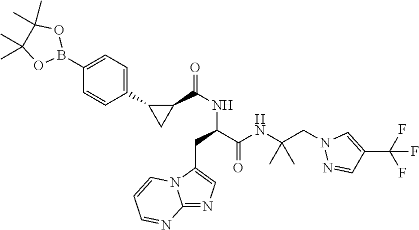 [SMILES: CC(C)(Cn1cc(C(F)(F)F)cn1)NC(=O)[C@@H](Cc1cnc2ncccn12)NC(=O)[C@H]1C[C@@H]1c1ccc(B2OC(C)(C)C(C)(C)O2)cc1]

A mixture of PdCl₂(dppf) (DCM adduct, 102 mg, 125 μmol), bis(pinacolato)diboron (790 mg, 3.11 mmol), KOAc (489 mg, 4.98 mmol), and (1S,2S)-2-(4-bromophenyl)-N—((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl)cyclopropane-1-carboxamide (770 mg, 1.25 mmol) in 1,4-dioxane (8.3 mL) was sparged with N₂ for 5 min, then heated to 80° C. for 2 h. The reaction mixture was filtered and carried forward as a solution of the title compound (approximately 0.15 M, quantitative yield). ES-MS m/z 666 (M+H).

The compounds in the table below were prepared as described in Preparation 200 using the appropriate aryl bromide.

TABLE 15

| | | | ES-MS |
|---|---|---|---|
| Preparation | Chemical name | Structure | m/z |
| 202 | (1S,2S)-N-((R)-1-((1-(4- Fluorophenyl)-2-methyl- propan-2-yl)amino)-3- (imidazo[1,2-a]pyrimidin- 3-yl)-1-oxopropan-2-yl)- 2-(4-(4,4,5,5-tetramethyl- 1,3,2-dioxaborolan-2-yl)- phenyl)cyclopropane-1- carboxamide | 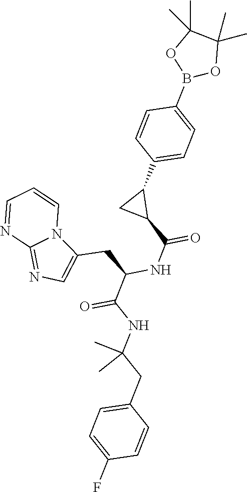 [SMILES: CC(C)(Cc1ccc(F)cc1)NC(=O)[C@@H](Cc1cnc2ncccn12)NC(=O)[C@H]1C[C@@H]1c1ccc(B2OC(C)(C)C(C)(C)O2)cc1] | 626 (M + H) |
| 203 | (1S,2S)-N-Methyl-N- ((R)-1-((2-methyl-1-(5- (1-methyl-6-oxo-1,6- dihydropyridin-3-yl)-3- (trifluoromethyl)-1H- pyrazol-1-yl)propan-2- yl)amino)-3-(1-methyl- 1H-pyrazol-4-yl)-1-oxo- propan-2-yl)-2-(4- (4,4,5,5-tetramethyl- 1,3,2-dioxaborolan-2- yl)phenyl)cyclopropane- 1-carboxamide | 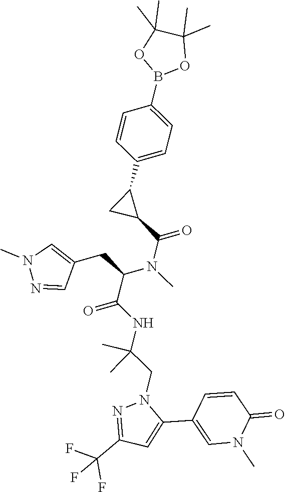 [SMILES: CN(C(=O)[C@H]1C[C@@H]1c1ccc(B2OC(C)(C)C(C)(C)O2)cc1)[C@H](Cc1cnn(C)c1)C(=O)NC(C)(C)Cn1nc(C(F)(F)F)cc1-c1ccc(=O)n(C)c1] | 750 (M + H) |
| 204 | (1S,2S)-N-((R)-1-((1-(5- Fluoropyridin-2-yl)-2- methylpropan-2-yl)- amino)-3-(imidazo[1,2-a] pyrimidin-3-yl)-1-oxo- propan-2-yl)-2-(4- (4,4,5,5-tetramethyl- 1,3,2-dioxaborolan-2-yl)- phenyl)cyclopropane-1- carboxamide | 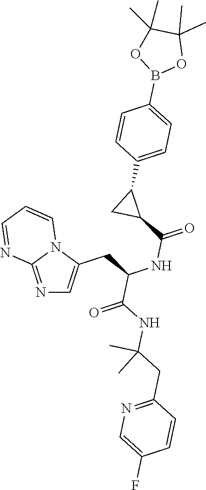 [SMILES: CC(C)(Cc1ccc(F)cn1)NC(=O)[C@@H](Cc1cnc2ncccn12)NC(=O)[C@H]1C[C@@H]1c1ccc(B2OC(C)(C)C(C)(C)O2)cc1] | 627 (M + H) |

### Example 7

### (trans)-N—((R)-1-((1-(5-Bromo-3-(trifluoromethyl)-1H-pyrazol-1-yl)-2-methylpropan-2-yl)amino)-3-(1-methyl-1H-pyrazol-4-yl)-1-oxopropan-2-yl)-2-(5-(2-methyl-2H-pyrazolo[3,4-b]pyridin-5-yl) thiophen-2-yl)cyclopropane-1-carboxamide, Isomer 2

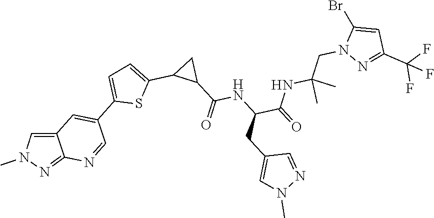 [SMILES: Cn1cc(C[C@@H](NC(=O)C2CC2c2ccc(-c3cnc4nn(C)cc4c3)s2)C(=O)NC(C)(C)Cn2nc(C(F)(F)F)cc2Br)cn1]

To a mixture of (2-methyl-2H-pyrazolo[3,4-b]pyridin-5-yl) boronic acid (76 mg, 0.43 mmol) and (trans)-N—((R)-1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)-2-(5-bromothiophen-2-yl)cyclopropane-1-carboxamide (mixture of 2 diastereomers, 280 mg, 420 μmol), Pd(dppf)Cl₂·CH₂Cl₂ (18 mg, 25 μmol), and Cs₂CO₃ (250 mg, 767 μmol) was added 3:1 1,4-dioxane/water (5 mL), and the resulting mixture was stirred at RT for 5 min, then at 90° C. for 1 h. The reaction mixture was cooled to RT, diluted with 1,4-dioxane (6 mL) and EtOAc (6 mL), and stirred. The layers were separated, and the aq. layer was acidified to pH3 with 1N aq. HCl (˜0.7 mL) before extracting with EtOAc (3×30 mL). All organic layers were combined, dried over Na₂SO₄, and concentrated under reduced pressure. The residue was purified by reversed phase prep-HPLC (Waters® XSelect® CSH C18, 30×150 mm, 5 μm, 60 mL/min) eluting with 25-50% ACN in 10 mM aq. NH₄HCO₃, pH=10 (with 5% MeOH) over 20 min to obtain the second eluting isomer as the title compound (100 mg, 31%). ES-MS m/z 718, 720 (M+H).

### Example 8

### (1S,2S)-2-(4-(7-Methyl-1,8-naphthyridin-3-yl)phenyl)-N-((2R)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-3-(1-methyl-1H-pyrazol-4-yl)-1-oxopropan-2-yl)cyclopropane-1-carboxamide

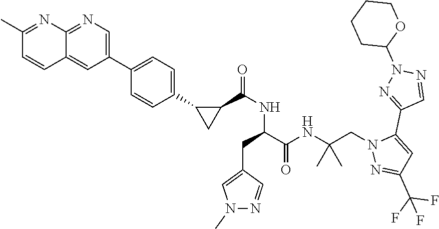 [SMILES: Cc1ccc2cc(-c3ccc([C@H]4C[C@@H]4C(=O)N[C@H](Cc4cnn(C)c4)C(=O)NC(C)(C)Cn4nc(C(F)(F)F)cc4-c4cnn(C5CCCCO5)n4)cc3)cnc2n1]

To a mixture of 6-bromo-2-methyl-1,8-naphthyridine (15 mg, 67 μmol), (1S,2S)-N—((R)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-3-(1-methyl-1H-pyrazol-4-yl)-1-oxopropan-2-yl)-2-(4-(4,4,5,5-tetramethyl-1,3,2-dioxaborolan-2-yl)phenyl)cyclopropane-1-carboxamide (40 mg, 45 μmol, 87% purity), Cs₂CO₃ (44 mg, 0.14 mmol) and Pd(dppf)Cl₂ (10 mg, 14 μmol) under N₂, was added N₂ sparged 4:1 1,4-dioxane/water (1 mL), and the resulting mixture was stirred at 90° C. for 2 h. The reaction mixture was cooled to RT, quenched with water, then extracted with EA. The layers were separated, and the organic layer was dried over Na₂SO₄, filtered, and concentrated under reduced pressure to obtain the title compound (100 mg, 31%) as a dark-brown oil. ES-MS m/z 718.2/720.1 (M+H).

### Preparation 205

### (1S,2S)-2-(4-Aminophenyl)-N—((R)-1-((1-(4-fluorophenyl)-2-methylpropan-2-yl)amino)-3-(1-methyl-1H-pyrazol-4-yl)-1-oxopropan-2-yl)cyclopropane-1-carboxamide hydrochloride

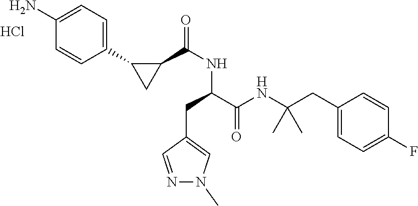 [SMILES: Cl ; Cn1cc(C[C@@H](NC(=O)[C@H]2C[C@@H]2c2ccc(N)cc2)C(=O)NC(C)(C)Cc2ccc(F)cc2)cn1]

A solution of tert-butyl (4-((1S,2S)-2-(((R)-1-((1-(4-fluorophenyl)-2-methylpropan-2-yl)amino)-3-(1-methyl-1H-pyrazol-4-yl)-1-oxopropan-2-yl) carbamoyl)cyclopropyl)phenyl)carbamate (380 mg, 559 μmol, 85% purity) in MeOH (1 mL) and 1,4-dioxane (2 mL) was treated with 4M HCl in 1,4-dioxane (1.40 mL, 5.59 mmol). The resulting mixture was stirred at 25° C. for 4 h then concentrated under reduced pressure to obtain a quantitative yield of the title compound (390 mg, 75% purity) as yellow solid. ES-MS m/z 478.1 (M+H).

### Preparation 206

### (1R,2S)-1-(2-Chloroethyl)-N—((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)-2-(2-fluoro-4-(2-methyl-2H-pyrazolo[3,4-b]pyridin-5-yl)phenyl)cyclopropane-1-carboxamide

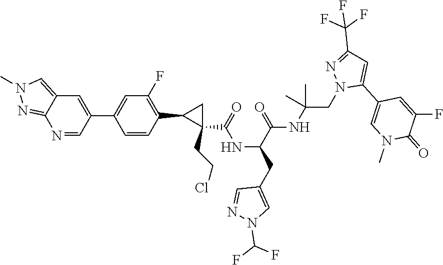 [SMILES: Cn1cc2cc(-c3ccc([C@H]4C[C@]4(CCCl)C(=O)N[C@H](Cc4cnn(C(F)F)c4)C(=O)NC(C)(C)Cn4nc(C(F)(F)F)cc4-c4cc(F)c(=O)n(C)c4)c(F)c3)cnc2n1]

A mixture of (1S,3R)-1-(2-fluoro-4-(2-methyl-2H-pyrazolo[3,4-b]pyridin-5-yl)phenyl)-5-oxaspiro[2.4]heptan-4-one (200 mg, 593 μmol) and benzyltriethylammonium chloride (270 mg, 1.19 mmol) in DCE (5 mL) was treated with SOCl₂ (176 μL, 2.39 mmol) followed by BF₃·Et₂O (148 μL, 1.18 mmol). After stirring at 80° C. overnight, the reaction mixture was cooled to RT and concentrated under reduced pressure. DCE (5 mL) was added, and the mixture was concentrated under reduced pressure to obtain a quantitative yield of (1R,2R)-1-(2-chloroethyl)-2-(2-fluoro-4-(2-methyl-2H-pyrazolo[3,4-b]pyridin-5-yl)phenyl)cyclopropane-1-carbonyl chloride as a light-brown solid. In a separate vessel, a mixture of (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide hydrochloride (350 mg, 591 μmol) in DCM (10 mL) was stirred at 0° C. and treated with TEA (579 μL, 4.15 mmol). A suspension of (1R,2R)-1-(2-chloroethyl)-2-(2-fluoro-4-(2-methyl-2H-pyrazolo[3,4-b]pyridin-5-yl)phenyl)cyclopropane-1-carbonyl chloride (593 μmol) in DCM (10 mL) was added dropwise. The cooling bath was removed, and the reaction mixture was stirred at RT for 30 min. EtOAc (100 mL) and cold water (100 mL) were added, and the layers were separated. The aq. layer was extracted with EtOAc (100 mL), and the organic layers were combined, washed with water (3×100 mL), dried over Na₂SO₄, filtered, and concentrated under reduced pressure to obtain a quantitative yield of the title compound as a light-brown solid. ES-MS m/z 875 (M+H).

The compounds in the following table were prepared as described in Preparation 206 using the appropriate lactone and appropriate amine or amine salt. DIEA is a suitable replacement for TEA. Slight variations to the reaction conditions and purification methods would be apparent to one skilled in the art.

TABLE 16

| | | | ES-MS |
|---|---|---|---|
| Prep | Chemical Name | Structure | m/z |
| 207ª | (1R,2R)-N-((R)-1-((1-(5- Bromo-3-(trifluorometh- yl)-1H-pyrazol-1-yl)-2- methylpropan-2-yl)- amino)-3-(1-(difluoro- methyl)-1H-pyrazol-4- yl)-1-oxopropan-2-yl)-1- (2-chloroethyl)-2-(4-(2- methyl-2H-pyrazolo[3,4- b]pyridin-5-l)phenyl)- cyclopropane-1-carbox- amide | 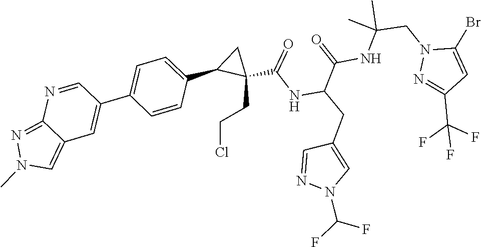 [SMILES: Cn1cc2cc(-c3ccc([C@H]4C[C@]4(CCCl)C(=O)NC(Cc4cnn(C(F)F)c4)C(=O)NC(C)(C)Cn4nc(C(F)(F)F)cc4Br)cc3)cnc2n1] | 810, 812 (M + H) |
| 208^{b} | (1R,2R)-1-(2-chloroeth- yl)-N-((2R)-1-((2-methyl- 1-(5-(tetrahydro-2H- pyran-2-yl)-3-(trifluoro- methyl)-1H-pyrazol-1-yl)- propan-2-yl)amino)-3-(1- methyl-1H-pyrazol-4-yl)- 1-oxopropan-2-yl)-2-(4- (2-methyl-2H-pyrazolo [3,4-b]pyridin-5-yl)phen- yl)cyclopropane-1-carbox- amide | 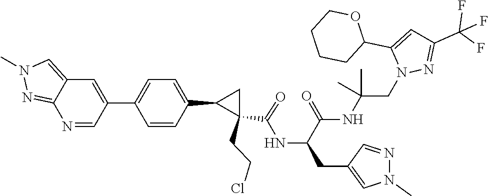 | 847 (M + H) |
| 209^{c} | (1R,2R)-1-(2-chloroeth- yl)-N-((R)-1-((2-methyl- 1-(5-(1-methyl-6-oxo- 1,6-dihydropyridin-3-yl)- 3-(trifluoromethyl)-1H- pyrazol-1-yl)propan-2- yl)amino)-3-(1-methyl- 1H-pyrazol-4-yl)-1-oxo- propan-2-yl)-2-(4-(2- methyl-2H-pyrazolo[3,4- b]pyridin-5-l)phenyl) cyclopropane-1-carbox- amide | 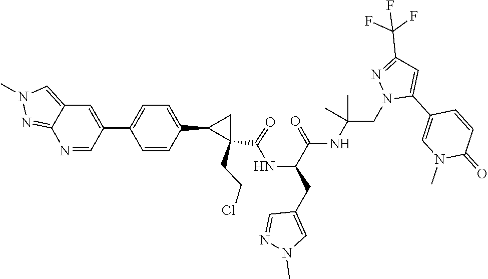 [SMILES: Cn1cc(C[C@@H](NC(=O)[C@@]2(CCCl)C[C@@H]2c2ccc(-c3cnc4nn(C)cc4c3)cc2)C(=O)NC(C)(C)Cn2nc(C(F)(F)F)cc2-c2ccc(=O)n(C)c2)cn1] | 803 (M + H) |

^{a.}purification on silica with 0-15% MeOH in EtOAc.
^{b.}purification on silica with 0-5% MeOH in DCM.
^{c.}used DIEA instead of TEA, purification on silica with 0-40% MeOH in DCM.

### Example 9

### (2R)—N-(2-Methyl-1-(5-(2-(tetrahydro-2H-pyran-2-yl)-2H-1,2,3-triazol-4-yl)-3-(trifluoromethyl)-1H-pyrazol-1-yl)propan-2-yl)-3-(1-methyl-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide

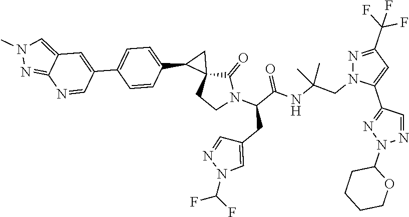 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4cnn(C6CCCCO6)n4)C5=O)cc3)cnc2n1]

A mixture of (1R,2R)-1-(2-chloroethyl)-N-((2R)-1-((2-methyl-1-(5-(tetrahydro-2H-pyran-2-yl)-3-(trifluoromethyl)-1H-pyrazol-1-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide (338 mg, 399 μmol) and Cs₂CO₃ (520 mg, 1.60 mmol) in NMP (8 mL) was stirred for 18 h at RT. The reaction mixture was added dropwise slowly to rapidly stirring water (150 mL). Suction filtration of the resulting mixture gave a quantitative yield of the title compound (340 mg) as a white solid. ES-MS m/z 811.2 (M+H).

### Example 10

### (R)—N-(1-(5-Bromo-3-(trifluoromethyl)-1H-pyrazol-1-yl)-2-methylpropan-2-yl)-3-(1-(difluoromethyl)-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide

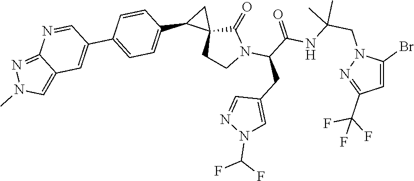 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4Br)C5=O)cc3)cnc2n1]

The title compound was prepared as described in Example 9 using (1R,2R)—N—((R)-1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)-1-(2-chloroethyl)-2-(4-(2-methyl-2H-pyrazolo[3,4-b]pyridin-5-yl)phenyl)cyclopropane-1-carboxamide and stirring for 5 days, followed by reversed phase flash purification (150 g, C18 using a gradient of 50-100% ACN in water (with 0.1% FA). ES-MS m/z 774,776 (M+H).

### Preparation 210

### N-(2,2-Dimethoxyethyl)-N-methyl-1H-pyrazole-5-carboxamide

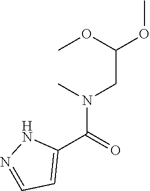

To a solution of 1H-pyrazole-5-carboxylic acid (7.00 g, 62.4 mmol), 2,2-dimethoxy-N-methylethan-1-amine (8.19 g, 68.7 mmol) and HATU (30.9 g, 81.2 mmol) in DMA (70.0 mL) was added DIEA (43.0 mL, 250 mmol) at 0° C. The reaction was stirred at 26° C. for 16 h and then diluted with water (300 mL). The reaction mixture was extracted with EtOAc (3×300 mL), and the combined organic layers were washed with sat. aq. NaCl (3× 300 mL), dried over Na₂SO₄, filtered, and concentrated under reduced pressure. The residue was purified by silica gel chromatography eluting with 0-60% EtOAc in heptane to obtain the title compound (14.5 g, 54%, 50% purity) as a yellow oil. ES-MS m/z 214 (M+H).

### Preparation 211

### 5-Methylpyrazolo[1,5-a]pyrazin-4 (5H)-one

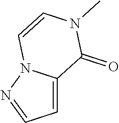

A mixture of N-(2,2-dimethoxyethyl)-N-methyl-1H-pyrazole-5-carboxamide (14.5 g, 33.9 mmol, 50 wt %) and p-toluenesulfonic acid (6.45 g, 33.9 mmol) in toluene (100 mL) was stirred at 130° C. for 16 h. Upon conversion, the pH of the mixture was adjusted to ˜8 with saturated aq. NaHCO₃, then the mixture was diluted with water (150 mL) and extracted with DCM (3× 100 mL). The combined organic layers were dried over Na₂SO₄, filtered and concentrated under reduced pressure. The residue was purified by silica gel chromatography using a gradient of 0-57% EtOAc in heptane to obtain the title compound (6.00 g, 95%, 80% purity) as a white solid. ES-MS m/z 150.0 (M+H).

### Preparation 212

### 7-Bromo-5-methylpyrazolo[1,5-a]pyrazin-4 (5H)-one

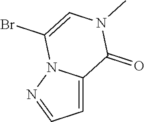

To a mixture of 5-methylpyrazolo[1,5-a]pyrazin-4 (5H)-one (2.00 g, 80 wt %, 10.7 mmol) in DMF (20.0 mL) was added NBS (2.10 g, 11.8 mmol). The reaction mixture was degassed and refilled with N₂×3, cooled to 0° C. and then stirred at 0° C. for 1 h under N₂. The reaction mixture was then diluted with water (100 mL) and extracted with EtOAc (3×100 mL). The combined organic layers were washed with sat. aq. NaCl (2×100 mL), dried over Na₂SO₄, filtered, and concentrated under reduced pressure. The residue was purified by silica gel chromatography using a gradient of 0-32% EtOAc in heptane to obtain the title compound (1.31 g, 50%) as a white solid. ES-MS m/z 228,230 (M+H)

### Preparation 213

### tert-Butyl (1-(5-(1,3,6,2-dioxazaborocan-2-yl)-3-(trifluoromethyl)-1H-pyrazol-1-yl)-2-methylpropan-2-yl)carbamate

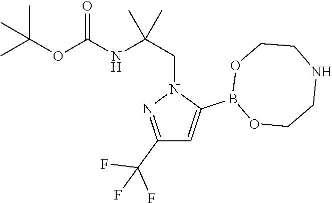 [SMILES: CC(C)(Cn1nc(C(F)(F)F)cc1B1OCCNCCO1)NC(=O)OC(C)(C)C]

A mixture of 1-(2-((tert-butoxycarbonyl)amino)-2-methylpropyl)-3-(trifluoromethyl)-1H-pyrazol-5-yl) boronic acid (9.3 g, 24 mmol) in 2-MeTHF (25 mL) was stirred at RT under nitrogen and a solution of 2,2′-azanediylbis(ethan-1-ol) (2.47 g, 23.5 mmol) in IPA (2.5 mL) was added dropwise. The resulting mixture was stirred at RT for 1 h. Then the reaction mixture was concentrated under reduced pressure. The residue was triturated with MTBE (50 mL) and 2-MeTHF (10 mL) at RT for 1 h, filtered and dried under reduced pressure to obtain the title compound (6.8 g, 66%) as a white solid. 1H NMR (400 MHz, DMSO-d₆) δ 7.42 (br s, 1H), 7.28 (br s, 1H), 6.56 (s, 1H), 4.25 (s, 2H), 3.88 (td, J=9.2, 5.6 Hz, 2H), 3.76 (ddd, J=9.6, 6.4, 3.6 Hz, 2H), 3.12-3.26 (m, 2H), 2.84-3.02 (m, 2H), 1.34 (s, 9H), 1.25 (s, 6H).

### Preparation 214

### 3-Fluoro-1-(methyl-d₃)-5-(4,4,5,5-tetramethyl-1,3,2-dioxaborolan-2-yl)pyridin-2(1H)-one

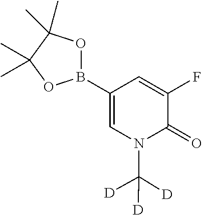 [SMILES: [2H]C([2H])([2H])n1cc(B2OC(C)(C)C(C)(C)O2)cc(F)c1=O]

To a stirring suspension of 3-fluoro-5-(4,4,5,5-tetramethyl-1,3,2-dioxaborolan-2-yl)pyridin-2(1H)-one (193 mg, 807 μmol) and Potassium carbonate (277 mg, 2.00 mmol) in DMF (1 mL), iodomethane-d₃ (233 mg, 1.61 mmol) was added. The resulting mixture was stirred at RT for 18 h under air, diluted with water (10 mL) and extracted with EtOAc (10 mL). The organic layer was washed with sat. aq. NaCl (10 mL) and dried over anhydrous sodium sulfate, filtered and concentrated under reduced pressure to obtain the title compound (35 mg, 17%) as a beige solid. ES-MS m/z 257.2 (M+H).

### Preparation 215

### tert-Butyl (2-methyl-1-(5-(4,4,5,5-tetramethyl-1,3,2-dioxaborolan-2-yl)-3-(trifluoromethyl)-1H-pyrazol-1-yl)propan-2-yl)carbamate

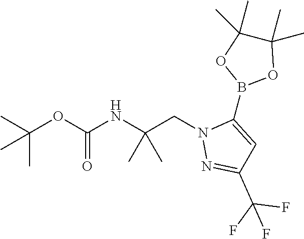 [SMILES: CC(C)(Cn1nc(C(F)(F)F)cc1B1OC(C)(C)C(C)(C)O1)NC(=O)OC(C)(C)C]

In a N₂-filled glovebox, bis(pinacolato) diborane (5.5 g, 22 mmol), [Ir(OMe)(1,5-cod)]₂ (0.36 g, 0.55 mmol) and 4,4′-bis(tert-butyl)-2,2′-bipyridine (0.29 g, 1.1 mmol) were added into a dried reaction vial. Degassed 2-MeTHF (40 mL) was added and the reaction mixture was stirred at 25° C. for 30 min. Then, a solution of tert-butyl (2-methyl-1-(3-(trifluoromethyl)-1H-pyrazol-1-yl)propan-2-yl)carbamate (5.7 g, 18 mmol) in 2-MeTHF (60 mL) was added to the catalyst solution. The resulting reaction mixture was stirred at 70° C. for 16 h. The reaction mixture was filtered and concentrated under reduced pressure. The residue was purified by silica gel chromatography using a gradient of 0-1% MeOH in DCM to obtain the title compound (7.1 g, 86%) as a white solid. ES-MS m/z 434.3 (M+H).

### Preparation 216

### tert-Butyl (1-(5-(5-fluoro-1-methyl-6-oxo-1,6-dihydropyridin-3-yl)-3-(trifluoromethyl)-1H-pyrazol-1-yl)-2-methylpropan-2-yl)carbamate

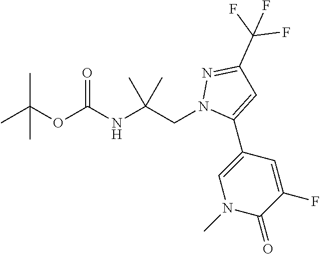 [SMILES: Cn1cc(-c2cc(C(F)(F)F)nn2CC(C)(C)NC(=O)OC(C)(C)C)cc(F)c1=O]

The title compound was prepared as described in Preparation 15 using tert-butyl (1-(5-bromo-3-(trifluoromethyl)-1H-pyrazol-1-yl)-2-methylpropan-2-yl)carbamate, 3-fluoro-1-methyl-5-(4,4,5,5-tetramethyl-1,3,2-dioxaborolan-2-yl)pyridin-2(1H)-one (0.95 eq), Na₂CO₃ (3 eq) and Pd(dppf)Cl₂ (0.2 eq) at 90° C. for 12 h. The reaction mixture was cooled down to RT, filtered and the filter cake was washed with EtOAc. The filtrate was concentrated under reduced pressure. The residue was purified by silica gel chromatography using a gradient of 0-40% EtOAc in PE. The residue was purified by silica gel chromatography using a gradient of 0-45% EtOAc in DCM to obtain the title compound (7.1 g, 86%) as a white solid. ES-MS m/z 433.0 (M+H).

### Preparation 217

### tert-Butyl (1-(5-(1-(cyanomethyl)-2-oxo-1,2-dihydropyridin-3-yl)-3-(trifluoromethyl)-1H-pyrazol-1-yl)-2-methylpropan-2-yl)carbamate

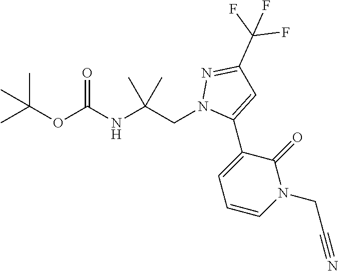 [SMILES: CC(C)(Cn1nc(C(F)(F)F)cc1-c1cccn(CC#N)c1=O)NC(=O)OC(C)(C)C]

To a solution of (1-(2-((tert-butoxycarbonyl)amino)-2-methylpropyl)-3-(trifluoromethyl)-1H-pyrazol-5-yl) boronic acid (75 mg, 88 wt %, 0.19 mmol), 2-(3-bromo-2-oxopyridin-1 (2H)-yl) acetonitrile (81 mg, 0.38 mmol), and Pd(dppf)Cl₂ (18 mg, 28 μmol) in 1,4-dioxane (1.3 mL) was added K₃PO₄ (2.5 M aq. solution, 0.26 mL, 0.66 mmol). The resulting mixture was sparged with argon for 2 min, then stirred at 90° C. for 1.5 h. The reaction mixture was concentrated under a stream of nitrogen, then diluted with DCM (5 mL). To the resulting mixture was added both SiliaMetS-thiol (60 mg) and SiliaMetS-Thiourea (60 mg) palladium scavengers. The mixture was shaken at 40° C. for 30 min, then filtered through a 3 g diatomaceous earth SPE cartridge. The cartridge was rinsed with DCM (20 mL) and EtOAc (5 mL). The filtrate was concentrated under a stream of nitrogen. The residue was purified by reverse phase prep-HPLC (Phenomenex Kinetex EVO C18, 30×100 mm, 5 μm) eluting with 33-67% ACN in water (with 0.1% FA) over 6 min to obtain the title compound (59 mg, 68%) as a brown solid. ES-MS m/z 340.2 (M+H-Boc).

The compounds in the following table were prepared as described in Preparation 217 using the appropriate aryl halide coupling partners. Reactants can be added in different orders or in differing equivalency. K₂CO₃ is a suitable replacement of K₃PO₄. Reaction times may vary, and differing methods can be used to work up or purify the compounds (normal phase, high or low pH prep-HPLC). Such variances would be apparent to one skilled in the art.

TABLE 17

| Preparation | Chemical Name | Structure | ES-MS m/z |
|---|---|---|---|
| 218^{a,b} | tert-Butyl (2-methyl-1-(5- (6-oxo-1-(2,2,2-trifluoro- ethyl)-1,6-dihydropyridin- 3-yl)-3-(trifluoromethyl)- 1H-pyrazol-1-yl)propan- 2-yl)carbamate | 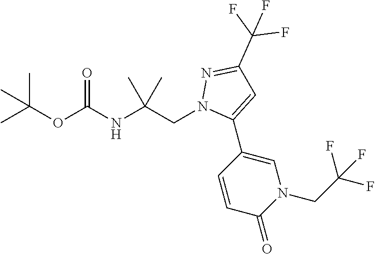 [SMILES: CC(C)(Cn1nc(C(F)(F)F)cc1-c1ccc(=O)n(CC(F)(F)F)c1)NC(=O)OC(C)(C)C] | 427 (M + H − tBu) |
| 219ª | tert-Butyl (1-(5-(2,5- difluoro-4-hydroxymeth- yl)phenyl)-3-(trifluoro- methyl)-1H-pyrazol-1- yl)-2-methylpropan-2- yl)carbamate | 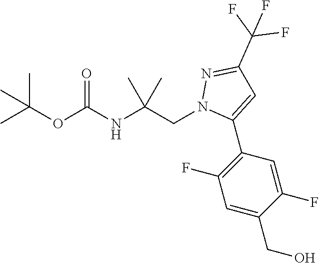 [SMILES: CC(C)(Cn1nc(C(F)(F)F)cc1-c1cc(F)c(CO)cc1F)NC(=O)OC(C)(C)C] | 394 (M + H − tBu) |
| 220^{a,b} | tert-Butyl (1-(5-(5-cyano- 1,4-dimethyl-6-oxo-1,6- dihydropyridin-3-yl)-3- (trifluoromethyl)-1H- pyrazol-1-yl)-2-methyl- propan-2-yl)carbamate | 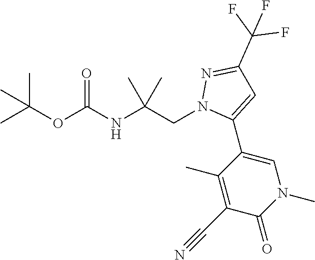 [SMILES: Cc1c(-c2cc(C(F)(F)F)nn2CC(C)(C)NC(=O)OC(C)(C)C)cn(C)c(=O)c1C#N] | 398 (M + H − tBu) |
| 221^{a,b} | tert-Butyl (2-methyl-1- (1′-(2-(methylamino)-2- oxoethyl)-5-(trifluoro- methyl)-1'H,2H-[3,4′- bipyrazol]-2-yl)propan- 2-yl)carbamate | 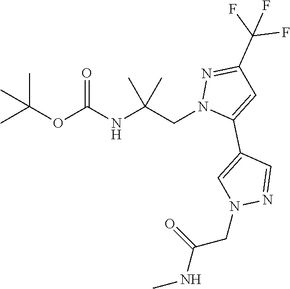 [SMILES: CNC(=O)Cn1cc(-c2cc(C(F)(F)F)nn2CC(C)(C)NC(=O)OC(C)(C)C)cn1] | 445 (M + H) |
| 222^{a,b} | tert-Butyl (2-methyl-1- (5-(1-methyl-6-oxo-5- (trifluoromethyl)-1,6- dihydropyridin-3-yl)-3- (trifluoromethyl)-1H- pyrazol-1-yl)propan-2- yl)carbamate | 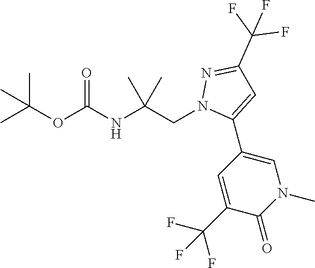 [SMILES: Cn1cc(-c2cc(C(F)(F)F)nn2CC(C)(C)NC(=O)OC(C)(C)C)cc(C(F)(F)F)c1=O] | 427 (M + H − tBu) |
| 223^{ª} | tert-Butyl (2-methyl-1- (5-(2-methyl-1-oxo-1,2- dihydroisoquinolin-4-yl)- 3-(trifluoromethyl)-1H- pyrazol-1-yl)propan-2- yl)carbamate | 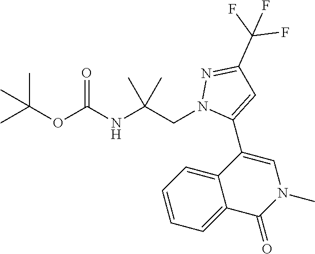 [SMILES: Cn1cc(-c2cc(C(F)(F)F)nn2CC(C)(C)NC(=O)OC(C)(C)C)c2ccccc2c1=O] | 409 (M + H − tBu) |
| 224 | tert-Butyl (1-(5-(6- (difluoromethoxy)pyr- idazin-3-yl)-3-(trifluoro- methyl)-1H-pyrazol-1- yl)-2-methylpropan-2- yl)carbamate | 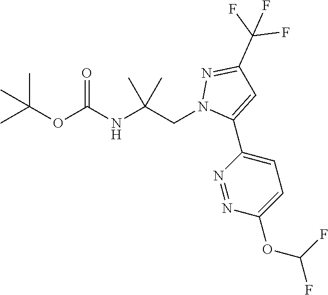 [SMILES: CC(C)(Cn1nc(C(F)(F)F)cc1-c1ccc(OC(F)F)nn1)NC(=O)OC(C)(C)C] | 352 (M + H − Boc) |
| 225^{c} | tert-Butyl (1-(5-(2-cyclo- propyl-4-hydroxy-6- methylpyrimidin-5-yl)-3- (trifluoromethyl)-1H- pyrazol-1-yl)-2-methyl- propan-2-yl)carbamate | 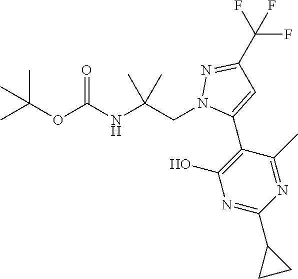 [SMILES: Cc1nc(C2CC2)nc(O)c1-c1cc(C(F)(F)F)nn1CC(C)(C)NC(=O)OC(C)(C)C] | 356 (M + H − Boc) |
| 226^{b,d} | tert-Butyl (1-(5-(1- (difluoromethyl)-2-oxo- 1,2-dihydropyridin-4-yl)- 3-(trifluoromethyl)-1H- pyrazol-1-yl)-2-methyl- propan-2-yl)carbamate | 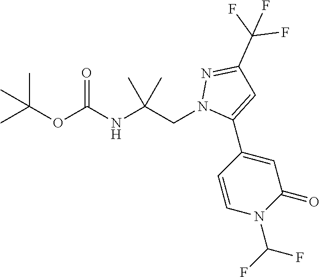 [SMILES: CC(C)(Cn1nc(C(F)(F)F)cc1-c1ccn(C(F)F)c(=O)c1)NC(=O)OC(C)(C)C] | 395 (M + H − tBu) |
| 227^{b} | tert-Butyl (1-(5-(6- ((dimethylamino)meth- yl)pyridin-3-yl)-3- (trifluoromethyl)-1H- pyrazol-1-yl)-2-meth- ylpropan-2-yl)carbamate | 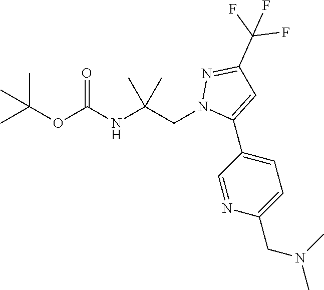 [SMILES: CN(C)Cc1ccc(-c2cc(C(F)(F)F)nn2CC(C)(C)NC(=O)OC(C)(C)C)cn1] | 442 (M + H) |

^{a.}2.5 eq of the aryl halide was used.
^{b.}The product was purified by reverse phase prep-HPLC (Phenomenex Kinetex EVO C18, 30 × 100 mm, 5 μm) eluting with a gradient of ACN in 10 mM aq. NH₄HCO₃ (with 5% MeOH).
^{c.}1.5 eq of the aryl halide was used, XPhos Pd G4 (0.1 eq) was used as catalyst, K₂CO₃ (3.0 eq) was used as base, and MeOH/H₂O (10:1) was used as solvent, and the reaction was heated to 70° C. for 2 h;
^{d.}3 eq K₃PO₄ (2.5M aq.) was used as base, and the reaction was heated to 90° C. for 3.5 h.

Preparation 228 tert-Butyl (1-(5-(8-fluoroimidazo[1,2-a]pyridin-6-yl)-3-(trifluoromethyl)-1H-pyrazol-1-yl)-2-methylpropan-2-yl)carbamate

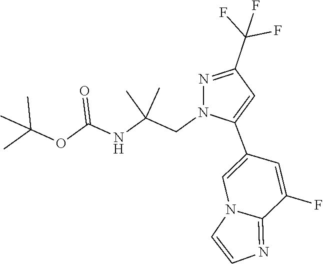 [SMILES: CC(C)(Cn1nc(C(F)(F)F)cc1-c1cc(F)c2nccn2c1)NC(=O)OC(C)(C)C]

A mixture of 6-bromo-8-fluoroimidazo[1,2-a]pyridine (39 mg, 0.18 mmol) and BrettPhos Pd G3 (17 mg, 19 μmol) in 1,4-dioxane (1.1 mL), IPA (300 μL) and Na₂CO₃ (1 M aq. solution, 0.50 mL, 0.50 mmol) was stirred at RT for 5 min. The mixture was purged with N₂ and tert-butyl (1-(5-(1,3,6,2-dioxazaborocan-2-yl)-3-(trifluoromethyl)-1H-pyrazol-1-yl)-2-methylpropan-2-yl)carbamate (70 mg, 0.17 mmol) was added. The mixture was heated at 65° C. for 16 h. Sat. aq. NaCl was added, then the mixture was extracted with EtOAc (3 mL). The organic layer was separated, filtered through diatomaceous earth, and concentrated under a stream of N₂ to provide the title compound (74 mg, quantitative yield). ES-MS m/z 442 (M+H).

### Preparation 229

### tert-Butyl (1-(5-(6-cyano-2-oxo-1,2-dihydropyridin-3-yl)-3-(trifluoromethyl)-1H-pyrazol-1-yl)-2-methylpropan-2-yl)carbamate

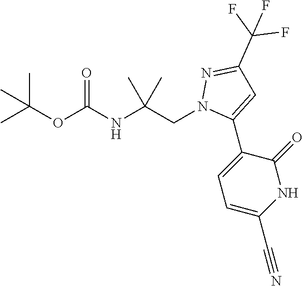 [SMILES: CC(C)(Cn1nc(C(F)(F)F)cc1-c1ccc(C#N)[nH]c1=O)NC(=O)OC(C)(C)C]

The title compound was prepared as described in Preparation 228 using 5-bromo-6-oxo-1,6-dihydropyridine-2-carbonitrile. ES-MS m/z 352 (M+H-Boc).

### Preparation 230

### tert-Butyl (2-methyl-1-(5-(1-(methyl-d₃)-6-oxo-1,6-dihydropyridin-3-yl)-3-(trifluoromethyl)-1H-pyrazol-1-yl)propan-2-yl)carbamate

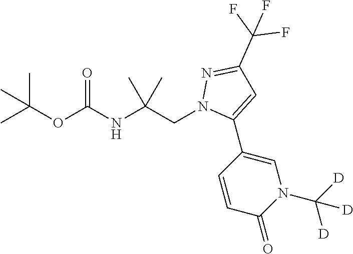 [SMILES: [2H]C([2H])([2H])n1cc(-c2cc(C(F)(F)F)nn2CC(C)(C)NC(=O)OC(C)(C)C)ccc1=O]

A mixture of XPhos Pd G4 (18 mg, 0.021 mmol), 5-bromo-1-(methyl-d₃)pyridin-2(1H)-one, and an aq. solution of K₃PO₄ (2.0 M, 0.38 mL, 0.76 mmol), then the vial was flushed with argon. An argon-sparged solution of (1-(2-((tert-butoxycarbonyl)amino)-2-methylpropyl)-3-(trifluoromethyl)-1H-pyrazol-5-yl) boronic acid (75 mg, 0.21 mmol) in 1,4-dioxane (1.9 mL) and EtOH (0.21 mL) was added to the reaction mixture. The resulting mixture was purged with argon for 10 min, then the reaction vessel was sealed and the mixture heated to 80° C. for 7 h. The reaction mixture was concentrated under a stream of nitrogen and the residue was purified by silica gel chromatography using a gradient of 0-35% 3:1 EtOAc/EtOH in heptane to obtain the title compound (54 mg, 60%) as a white solid. ES-MS m/z 362 (M+H).

### Preparation 231

### tert-Butyl (2-methyl-1-(5-(5-methyl-4-oxo-4,5-dihydropyrazolo[1,5-a]pyrazin-7-yl)-3-(trifluoromethyl)-1H-pyrazol-1-yl)propan-2-yl)carbamate

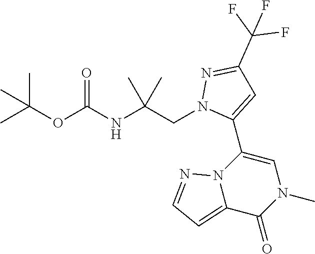 [SMILES: Cn1cc(-c2cc(C(F)(F)F)nn2CC(C)(C)NC(=O)OC(C)(C)C)n2nccc2c1=O]

The title compound was prepared as described in Preparation 230 using 7-bromo-5-methylpyrazolo[1,5-a]pyrazin-4 (5H)-one, 0.2 eq XPhos Pd G3 as catalyst, 1,4-dioxane/H₂O (10:1) as solvent, and the reaction was heated at 70° C. for 16 h. The product was purified by silica gel chromatography using a gradient of 0-50% EtOAc in hexanes. ES-MS m/z 355 (M+H-Boc).

### Preparation 232

### 5-(1-(2-Amino-2-methylpropyl)-3-(trifluoromethyl)-1H-pyrazol-5-yl)-3-fluoro-1-methylpyridin-2(1H)-one hydrochloride

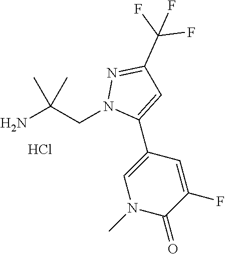 [SMILES: Cn1cc(-c2cc(C(F)(F)F)nn2CC(C)(C)N)cc(F)c1=O ; Cl]

A solution of tert-butyl (1-(5-(5-fluoro-1-methyl-6-oxo-1,6-dihydropyridin-3-yl)-3-(trifluoromethyl)-1H-pyrazol-1-yl)-2-methylpropan-2-yl)carbamate (23.5 g, 51.6 mmol) in 1,4-dioxane (50 mL) and MeOH (50 mL) was treated with 2M HCl in 1,4-dioxane (129 mL, 258 mmol). After stirring for 2 h at RT, the mixture was concentrated to obtain the title compound (19.0 g, quantitative yield) as a white solid. ES-MS (m/z) 333 (M+H).

The compounds in the following table were prepared as described in Preparation 232 using the appropriate tert-butyl carbamate. 4 M HCl in 1,4-dioxane could be a replacement of 2M HCl in 1,4-dioxane. Varying reaction times, eq of HCl, and solvents (DCM, MeOH) can be used, which would be apparent to one skilled in the art. The deprotected amines were isolated as the corresponding hydrochloride salt. The compounds may have been used without determination of the number of equivalents of HCl.

TABLE 18

| | | | ES-MS |
|---|---|---|---|
| Preparation | Chemical Name | Structure | m/z |
| 233^{a,b} | 5-(1-(2-Amino-2-methyl- propyl)-3-(trifluoromethyl)- 1H-pyrazol-5-yl)-1-(2,2,2- trifluoroethyl)pyridin-2(1H)- one hydrochloride | 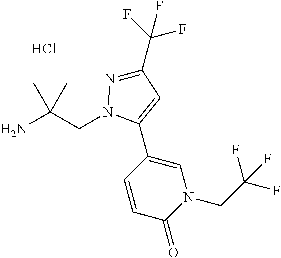 [SMILES: CC(C)(N)Cn1nc(C(F)(F)F)cc1-c1ccc(=O)n(CC(F)(F)F)c1 ; Cl] | 383 (M + H) |
| 234^{a,b} | (4-(1-(2-Amino-2-methyl- propyl)-3-(trifluoromethyl)- 1H-pyrazol-5-yl)-2,5- difluorophenyl)methanol hydrochloride | 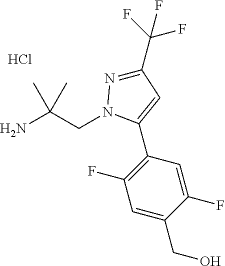 [SMILES: Cl ; CC(C)(N)Cn1nc(C(F)(F)F)cc1-c1cc(F)c(CO)cc1F] | 350 (M + H) |
| 235^{a,b,i} | 2-(3-(1-(2-Amino-2-meth- ylpropyl)-3-(trifluorometh- yl)-1H-pyrazol-5-yl)-2- oxopyridin-1(2H)-yl)acet- amide hydrochloride | 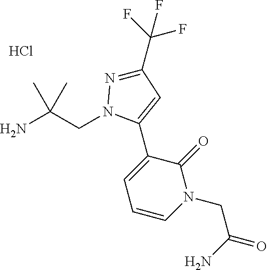 [SMILES: CC(C)(N)Cn1nc(C(F)(F)F)cc1-c1cccn(CC(N)=O)c1=O ; Cl] | 358 (M + H) |
| 236^{a,b} | 5-(1-(2-Amino-2-methyl- propyl)-3-(trifluoromethyl)- 1H-pyrazol-5-yl)-1,4- dimethyl-2-oxo-1,2-dihydro- pyridine-3-carbonitrile hydrochloride | 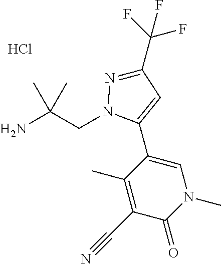 [SMILES: Cl ; Cc1c(-c2cc(C(F)(F)F)nn2CC(C)(C)N)cn(C)c(=O)c1C#N] | 354 (M + H) |
| 237^{a,b} | 2-(2-(2-Amino-2-methyl- propyl)-5-(trifluoromethyl)- 1′H,2H-[3,4′-bipyrazol]-1′- yl)-N-methylacetamide hydrochloride | 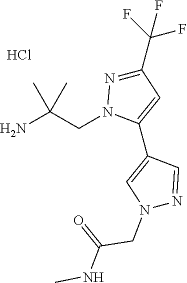 [SMILES: Cl ; CNC(=O)Cn1cc(-c2cc(C(F)(F)F)nn2CC(C)(C)N)cn1] | 345 (M + H) |
| 238^{a,b} | 5-(1-(2-Amino-2-methyl- propyl)-3-(trifluoromethyl)- 1H-pyrazol-5-yl)-1-methyl- 3-(trifluoromethyl)pyridin- 2(1H)-one hydrochloride | 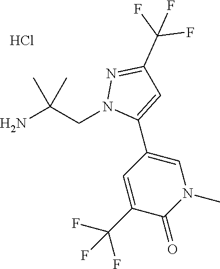 [SMILES: Cl ; Cn1cc(-c2cc(C(F)(F)F)nn2CC(C)(C)N)cc(C(F)(F)F)c1=O] | 383 (M + H) |
| 239^{a,b} | 4-(1-(2-Amino-2-methyl- propyl)-3-(trifluoromethyl)- 1H-pyrazol-5-yl)-2-methyl- isoquinolin-1(2H)-one hydrochloride | 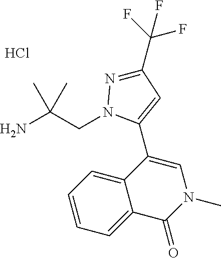 [SMILES: Cl ; Cn1cc(-c2cc(C(F)(F)F)nn2CC(C)(C)N)c2ccccc2c1=O] | 365 (M + H) |
| 240^{a,c} | 1-(5-(6-(Difluoromethoxy)- pyridazin-3-yl)-3-(trifluoro- methyl)-1H-pyrazol-1-yl)- 2-methylpropan-2-amine hydrochloride | 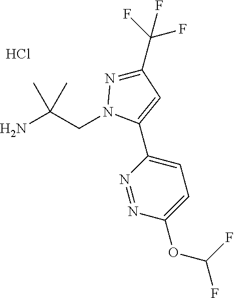 [SMILES: Cl ; CC(C)(N)Cn1nc(C(F)(F)F)cc1-c1ccc(OC(F)F)nn1] | 352 (M + H) |
| 241^{a,d} | 5-(1-(2-Amino-2-methyl- propyl)-3-(trifluoromethyl)- 1H-pyrazol-5-yl)-2-cyclo- propyl-6-methylpyrimidin- 4-ol hydrochloride | 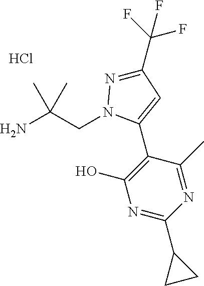 [SMILES: Cl ; Cc1nc(C2CC2)nc(O)c1-c1cc(C(F)(F)F)nn1CC(C)(C)N] | 356 (M + H) |
| 242^{a,e} | 7-(1-(2-Amino-2-methyl- propyl)-3-(trifluoromethyl)- 1H-pyrazol-5-yl)-5-methyl- pyrazolo[1,5-a]pyrazin- 4(5H)-one hydrochloride | 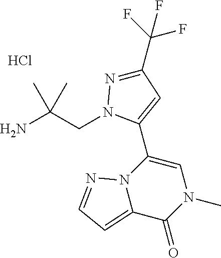 [SMILES: Cl ; Cn1cc(-c2cc(C(F)(F)F)nn2CC(C)(C)N)n2nccc2c1=O] | 355 (M + H) |
| 243^{a,f} | 4-(1-(2-Amino-2-methyl- propyl)-3-(trifluoromethyl)- 1H-pyrazol-5-yl)-1-(difluoro- methyl)pyridin-2(1H)-one hydrochloride | 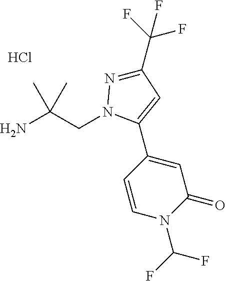 [SMILES: Cl ; CC(C)(N)Cn1nc(C(F)(F)F)cc1-c1ccn(C(F)F)c(=O)c1] | 351 (M + H) |
| 244^{a,g} | 1-(5-(6-((Dimethylamino)- methyl)pyridin-3-yl)-3- (trifluoromethyl)-1H-pyr- azol-1-yl)-2-methylpropan- 2-amine | 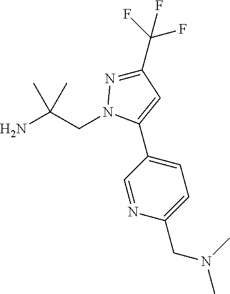 [SMILES: CN(C)Cc1ccc(-c2cc(C(F)(F)F)nn2CC(C)(C)N)cn1] | 342 (M + H) |
| 245^{a,h} | 5-(1-(2-Amino-2-methyl- propyl)-3-(trifluoromethyl)- 1H-pyrazol-5-yl)-1-(methyl- d₃)pyridin-2(1H)-one hydrochloride | 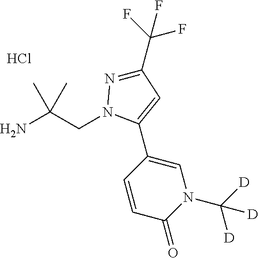 [SMILES: Cl ; [2H]C([2H])([2H])n1cc(-c2cc(C(F)(F)F)nn2CC(C)(C)N)ccc1=O] | 318 (M + H) |

^{a.}The volatiles were removed under a stream of nitrogen to obtain the product.
^{b.}Using 4M HCl in 1,4-dioxane (8 eq), DCM was used as solvent, 1 h reaction time.
^{c.}Using 4M HCl in 1,4-dioxane (30 eq), DCM was used as solvent, 2 h reaction time.
^{d.}Using 4M HCl in 1,4-dioxane (12 eq), DCM was used as solvent, 2 h reaction time.
^{e.}Using 2M HCl in 1,4-dioxane (30 eq), MeOH was used as solvent, 1 h reaction time. To the residue was added DCM and the mixture was stirred at 25º C. for 10 min. Then the mixture was filtered, the filter cake was washed with DCM (2 × 5 mL). The filter cake was dried in reduced pressure to give the product.
^{f.}Using 4M HCl in 1,4-dioxane (9 eq), DCM was used as solvent, 1.5 h reaction time.
^{g.}Using 4M HCl in 1,4-dioxane (18 eq), DCM was as solvent, 4 days reaction time. The volatiles were removed under a stream of nitrogen and the residue was dissolved in MeOH and loaded to a strong cation exchange SPE cartridge. The cartridge was eluted with MeOH and then 7M NH₃ in MeOH. The fractions containing the product were combined and concentrated under reduced pressure.
^{h.}Using 4M HCl in 1,4-dioxane (8 eq), MeOH was used as solvent, 5.5 h reaction time.
^{i.}The nitrile was converted to the primary amide under these reaction conditions.

### Preparation 246

### 2-(3-(1-(2-Amino-2-methylpropyl)-3-(trifluoromethyl)-1H-pyrazol-5-yl)-2-oxopyridin-1 (2H)-yl) acetonitrile

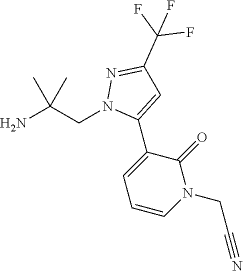 [SMILES: CC(C)(N)Cn1nc(C(F)(F)F)cc1-c1cccn(CC#N)c1=O]

A solution of tert-butyl (1-(5-(1-(cyanomethyl)-2-oxo-1,2-dihydropyridin-3-yl)-3-(trifluoromethyl)-1H-pyrazol-1-yl)-2-methylpropan-2-yl)carbamate (59 mg, 0.13 mmol) in DCM (4 mL) was treated with TFA (25 mL, 0.32 mmol), and the resulting mixture was stirred at RT for 2 h. TFA (1 drop) was added and the mixture was stirred at RT for 22 h. TFA (1 drop) was added and the mixture was stirred at RT for 2 h. The reaction was then quenched with NaHCO₃ (1 M) and the layers were separated. The aqueous layer was extracted twice with DCM (2.5 mL). The organic layers were combined, dried over Na₂SO₄, filtered, and concentrated under reduced pressure. The residue was placed in a vacuum oven at 50° C. for 30 min to provide the title compound (37 mg, 41% yield) as a white foam. ES-MS m/z 340 (M+H).

The compounds in the following table were prepared as described in Preparation 246 from the appropriate tert-butyl carbamate. Varying reaction times and eq of TFA, can be used, which would be apparent to one skilled in the art.

TABLE 19

| | | | ES-MS |
|---|---|---|---|
| Preparation | Chemical Name | Structure | m/z |
| 247ª | 1-(5-(8-Fluoroimidazo[1,2- a]pyridin-6-yl)-3-(trifluoro- methyl)-1H-pyrazol-1-yl)- 2-methylpropan-2-amine | 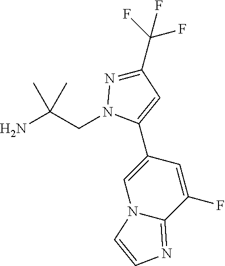 [SMILES: CC(C)(N)Cn1nc(C(F)(F)F)cc1-c1cc(F)c2nccn2c1] | 342.2 (M + H) |
| 248^{a,b} | 5-(1-(2-Amino-2-methyl- propyl)-3-(trifluorometh- yl)-1H-pyrazol-5-yl)-6- oxo-1,6-dihydropyridine- 2-carboxamide | 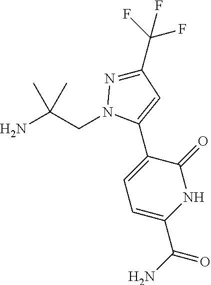 [SMILES: CC(C)(N)Cn1nc(C(F)(F)F)cc1-c1ccc(C(N)=O)[nH]c1=O] | 342.0 (M − H) |

^{a.}10 eq of TFA was used, 16 h reaction time, strong cation exchange was performed instead of aqueous workup.
^{b.}tert-Butyl (1-(5-(6-cyano-2-oxo-1,2-dihydropyridin-3-yl)-3-(trifluoromethyl)-1H-pyrazol-1-yl)-2-methylpropan-2-yl)carbamate was used as starting material-the nitrile was converted to the primary amide under these reaction conditions.

### Preparation 249

### 2-(6-Bromo-1,8-naphthyridin-2-yl)propan-2-ol

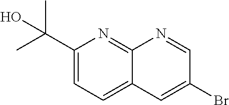

A mixture of 3-hydroxy-3-methylbutan-2-one (1.1 mL, 10 mmol) and 2-amino-5-bromonicotinaldehyde (2.0 g, 10 mmol) in EtOH (50 mL) was treated with NaOH (5.0 M aq. solution, 1.9 mL, 9.5 mmol). The mixture was heated to reflux and stirred for 1 h. Then the mixture was cooled down to RT, solvent was removed under reduced pressure. The resulting solid was washed with water and dried overnight under reduced pressure to obtain the title compound (2.3 g, 87%) as a yellow solid. ES-MS m/z 267,269 (M+H).

### Preparation 250

### 6-Bromo-3-methoxy-2-methyl-1,8-naphthyridine

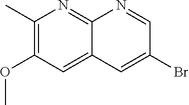

The title compound was prepared as described in Preparation 249 using 1-methoxypropan-2-one and 2-amino-5-bromonicotinaldehyde. ES-MS m/z 253,255 (M+H).

### Preparation 251

### tert-Butyl 3-(5-bromo-2H-pyrazolo[3,4-b]pyridin-2-yl) azetidine-1-carboxylate

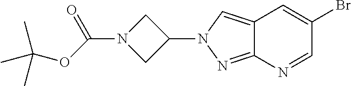 [SMILES: CC(C)(C)OC(=O)N1CC(n2cc3cc(Br)cnc3n2)C1]

A mixture of 5-bromo-1H-pyrazolo[3,4-b]pyridine (1.00 g, 5.05 mmol) in THF (15 mL) at 0° C. was treated with sodium bis(trimethylsilyl)amide solution (7.0 mL, 6.06 mmol, 1 M in THF) dropwise followed by tert-butyl 3-bromoazetidine-1-carboxylate (2.38 g, 10.1 mmol). The reaction mixture was allowed to warm to RT and stirred at RT for 3 days. NaI (757 mg, 5.05 mmol) was added followed by DMF (10 mL), and the resulting reaction mixture was stirred at RT for 3 days. Then the reaction mixture was heated at 70° C. overnight. The reaction mixture was cooled to RT, diluted with water, and extracted with EtOAc twice. The combined organic layers were washed with sat. aq. NaCl, dried over Na₂SO₄, filtered, and concentrated under reduced pressure. The residue was purified by silica gel chromatography using a gradient of 20-50% EtOAc in cyclohexane to obtain the title compound (second-eluting minor isomer, 410 mg, 23%) as an off-white solid. ES-MS m/z 353,355 (M+H).

### Preparation 252

### 2-(Azetidin-3-yl)-5-bromo-2H-pyrazolo[3,4-b]pyridine

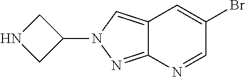 [SMILES: Brc1cnc2nn(C3CNC3)cc2c1]

A mixture of tert-butyl 3-(5-bromo-2H-pyrazolo[3,4-b]pyridin-2-yl) azetidine-1-carboxylate (360 mg, 1.02 mmol) in DCM (6 mL) was cooled at 0° C. and TFA (2 mL) was added dropwise at 0° C. The resulting mixture was allowed to warm to RT, stirred at RT for 2 h, and concentrated under reduced pressure. The residue was diluted with DCM, cooled down in an ice bath, and basified with sat. aq. NaHCO₃ to ˜pH 8. The layers were separated, and the aq. layer was extracted with DCM one more time and 3:1 CHCl₃/IPA three times. The organic layers were combined, dried over Na₂SO₄, filtered, and concentrated under reduced pressure to obtain the title compound (160 mg, 62%) as a white solid. ES-MS m/z 253,255 (M+H).

### Preparation 253

### 5-Bromo-2-(1-methylazetidin-3-yl)-2H-pyrazolo[3,4-b]pyridine

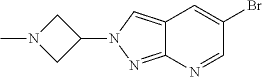

A mixture of 2-(azetidin-3-yl)-5-bromo-2H-pyrazolo[3,4-b]pyridine (160 mg, 632 μmol) in DCM (3 mL) and MeOH (3 mL) was stirred at 0° C. and formaldehyde (500 μL, 6.53 mmol, 36% in water) was added dropwise followed by HOAc (0.1 mL, 2 mmol). The resulting mixture was allowed to warm to RT and stirred at RT for 1 h. Then the reaction mixture was cooled down in an ice bath, and sodium triacetoxyborohydride (268 mg, 1.26 mmol) was added portion wise. After 10 min, the reaction mixture was basified with sat. aq. NaHCO₃ to pH 8. The resulting mixture was diluted with 50% MeOH in DCM, dried over anhydrous sodium sulfate, filtered and concentrated under reduced pressure. The obtained oil was dissolved in MeOH and loaded to 5 g strong cation exchange resin. The resin was eluted with MeOH and then 7 M NH₃ in MeOH. The fractions with desired product were combined and concentrated under reduced pressure to obtain the title compound (169 mg, quantitative yield) as a white solid. ES-MS m z 267,269 (M+H).

### Preparation 254

### 2-(5-Bromo-2H-pyrazolo[3,4-b]pyridin-2-yl) ethan-1-ol

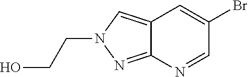 [SMILES: OCCn1cc2cc(Br)cnc2n1]

A mixture of 5-bromo-1H-pyrazolo[3,4-b]pyridine (200 mg, 1.01 mmol), 2-bromoethan-1-ol (252 mg, 2.02 mmol), NaI (151 mg, 1.01 mmol) and potassium tert-butoxide (170 mg, 1.52 mmol) in DMF (2 mL) was stirred at 140° C. overnight. The reaction mixture was cooled to RT, diluted with water (30 mL), and extracted with EtOAc (3×50 mL). The combined organic layers were washed with sat. aq. NaCl, dried over Na₂SO₄, filtered, and concentrated under reduced pressure. The residue was purified by reversed phase prep-HPLC (Kinetex® EVO C18, 30×100 mm, 5 μm, 85 mL/min) using a gradient of 5-39% ACN in 10 mM aq. NH₄HCO₃ (with 5% MeOH) to obtain the title compound (71 mg, 29%) as a white solid. ES-MS m z 242,244 (M+H).

### Preparation 255

### 2-Methyl-6-(4,4,5,5-tetramethyl-1,3,2-dioxaborolan-2-yl) imidazo[1,2-a]pyrimidine

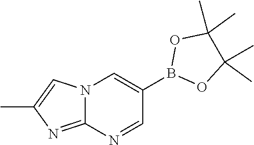

To a mixture of 6-bromo-2-methylimidazo[1,2-a]pyrimidine (0.300 g, 1.42 mmol), bis(pinacolato)diboron (1.46 g, 5.76 mmol), Pd(dppf)Cl₂·CH₂Cl₂ (0.172 g, 0.210 mmol), KOAc (0.422 g, 4.30 mmol) were added 1,4-dioxane (5.00 mL) and purged with N₂ for 5 mins. The resulting mixture was then stirred at 90° C. overnight under N₂. The reaction mixture was cooled to RT to give the title compound which was used as a solution. ES-MS m/z 260 (M+H).

### Preparation 256

### 5-(5-Chloro-4-(trifluoromethyl)pyrimidin-2-yl)-2-methyl-2H-pyrazolo[3,4-b]pyridine

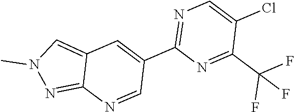 [SMILES: Cn1cc2cc(-c3ncc(Cl)c(C(F)(F)F)n3)cnc2n1]

2-Methyl-5-(4,4,5,5-tetramethyl-1,3,2-dioxaborolan-2-yl)-2H-pyrazolo[3,4-b]pyridine (700 mg, 2.70 mmol), Pd(dppf)Cl₂·CH₂Cl₂ (221 mg, 270 μmol), 2-bromo-5-chloro-4-(trifluoromethyl)pyrimidine (1.06 g, 4.05 mmol), and cesium carbonate (3.87 g, 11.9 mmol) were all combined in 1,4-dioxane/water (3:1) (10 mL). The suspension was sparged with N₂ for 5 min, then heated to 90° C. and held at this temperature for 1.0 h. The reaction was then cooled to RT, diluted with 12 mL of EtOAc and stirred. The organic extract was passed through sodium sulfate and 8 grams of Silicycle SiliMetS® Thiol metal scavenger, then concentrated under reduced pressure to obtain a quantitative yield of the title compound as a brown oil. ES-MS m/z 314 (M+H).

### Preparation 257

### 5-(5-Chloro-6-(difluoromethyl)pyridin-2-yl)-2-methyl-2H-pyrazolo[3,4-b]pyridine

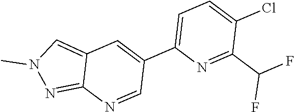 [SMILES: Cn1cc2cc(-c3ccc(Cl)c(C(F)F)n3)cnc2n1]

The title compound was prepared as described in Preparation 256 using 6-bromo-3-chloro-2-(difluoromethyl)pyridine (stirring for 2 h). ES-MS m/z 295 (M+H).

### Preparation 258

### Ethyl (1S,2S)-2-(2-(2-methyl-2H-pyrazolo[3,4-b]pyridin-5-yl)-4-(trifluoromethyl)pyrimidin-5-yl)cyclopropane-1-carboxylate

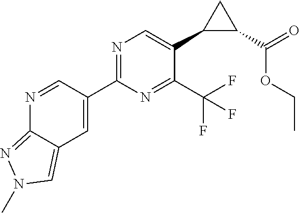 [SMILES: CCOC(=O)[C@H]1C[C@@H]1c1cnc(-c2cnc3nn(C)cc3c2)nc1C(F)(F)F]

Catacxium Pd G4 (200 mg, 270 μmol), 5-(5-chloro-4-(trifluoromethyl)pyrimidin-2-yl)-2-methyl-2H-pyrazolo[3,4-b]pyridine (847 mg, 2.70 mmol), ethyl (1S,2S)-2-(trifluoro-14-boraneyl)cyclopropane-1-carboxylate, potassium salt (1.19 g, 5.40 mmol) and cesium carbonate (2.64 g, 648 μL, 8.10 mmol) were combined in toluene (8 mL). The mixture was evacuated and backfilled with N₂, water (0.8 mL) was added, and then evacuated and backfilled again with N₂. The reaction vessel was sealed and heated to 80° C. for 16 h, then filtered and purified by reverse phase flash chromatography using a gradient of 30-100% ACN/10 mM aq. NH₄HCO₃ (w/5% MeOH) to obtain the title compound (887 mg, 84%) as a brown solid. ES-MS m/z 392 (M+H).

### Preparation 259

### Ethyl (1S,2S)-2-(2-(difluoromethyl)-6-(2-methyl-2H-pyrazolo[3,4-b]pyridin-5-yl)pyridin-3-yl)cyclopropane-1-carboxylate

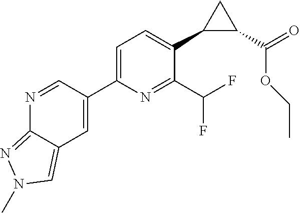 [SMILES: CCOC(=O)[C@H]1C[C@@H]1c1ccc(-c2cnc3nn(C)cc3c2)nc1C(F)F]

Prepared in a similar manner to Preparation 258 using 5-(5-chloro-6-(difluoromethyl)pyridin-2-yl)-2-methyl-2H-pyrazolo[3,4-b]pyridine (stirring for 7 h) to afford the title compound (238 mg, 21%) as a light brown solid. ES-MS m/z 373.0 (M+H).

### Preparation 260

### (1S,2S)-2-(2-(2-Methyl-2H-pyrazolo[3,4-b]pyridin-5-yl)-4-(trifluoromethyl)pyrimidin-5-yl)cyclopropane-1-carboxylic acid

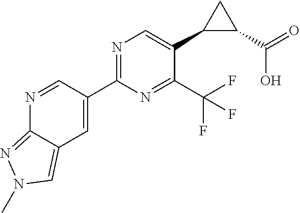 [SMILES: Cn1cc2cc(-c3ncc([C@H]4C[C@@H]4C(=O)O)c(C(F)(F)F)n3)cnc2n1]

Ethyl (1S,2S)-2-(2-(2-methyl-2H-pyrazolo[3,4-b]pyridin-5-yl)-4-(trifluoromethyl)pyrimidin-5-yl)cyclopropane-1-carboxylate (887 mg, 2.27 mmol) and lithium hydroxide hydrate (11.3 mL, 2.0 molar, 22.7 mmol) were combined in MeOH (2 mL) and THE (2 mL) and allowed to stir overnight. Hydrogen chloride in 1,4-dioxane (5.67 mL, 4.0 molar, 22.7 mmol) was added and the reaction mixture was concentrated under a stream of N₂ to obtain a quantitative yield of the title compound as a brown solid. ES-MS m/z 364.0 (M+H).

### Preparation 261

### (1S,2S)-2-(2-(Difluoromethyl)-6-(2-methyl-2H-pyrazolo[3,4-b]pyridin-5-yl)pyridin-3-yl)cyclopropane-1-carboxylic acid

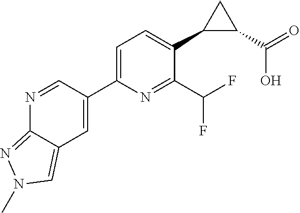 [SMILES: Cn1cc2cc(-c3ccc([C@H]4C[C@@H]4C(=O)O)c(C(F)F)n3)cnc2n1]

The title compound was prepared as described in Preparation 260 from ethyl (1S,2S)-2-(2-(difluoromethyl)-6-(2-methyl-2H-pyrazolo[3,4-b]pyridin-5-yl)pyridin-3-yl)cyclopropane-1-carboxylate (stirring for 3 h). ES-MS m/z 345.0 (M+H).

### Preparation 262

### rac-Ethyl (trans)-2-(4-bromo-2-fluorophenyl)-2-methylcyclopropane-1-carboxylate

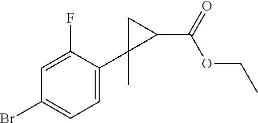 [SMILES: CCOC(=O)C1CC1(C)c1ccc(Br)cc1F]

To a 40 mL vial containing 4-bromo-2-fluoro-1-(prop-1-en-2-yl)benzene (1.54 g, 65% Wt, 4.65 mmol) was added a stirbar, rhodium (II) octanoate dimer (39 mg, 50 μmol), and DCM (3 mL). The reaction mixture was stirred at RT under argon as a solution of ethyl 2-diazoacetate (911 mg, 87% Wt, 6.95 mmol) in DCM (6 mL total volume) was added via syringe pump over 26 min. Upon complete addition, the reaction mixture was stirred for an additional hour at RT, then concentrated under Ar. The residue was purified by silica gel chromatography using a gradient of 0-20% MTBE in heptane, to provide the title compound (50 mg, 3.3%, first eluting diastereomer) as a colorless oil, followed by rac-ethyl (cis)-2-(4-bromo-2-fluorophenyl)-2-methylcyclopropane-1-carboxylate (252 mg, 18.0%, second-eluting diastereomer) as an orange oil. For both diastereomers, ES-MS m/z 301,303 (M+H).

### Preparation 263

### rac-Ethyl (trans)-2-(2-fluoro-4-(2-methyl-2H-pyrazolo[3,4-b]pyridin-5-yl)phenyl)-2-methylcyclopropane-1-carboxylate

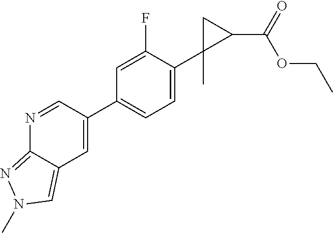 [SMILES: CCOC(=O)C1CC1(C)c1ccc(-c2cnc3nn(C)cc3c2)cc1F]

A 1-dram vial was charged with rac-ethyl (trans)-2-(4-bromo-2-fluorophenyl)-2-methylcyclopropane-1-carboxylate (50 mg, 92% wt, 0.15 mmol), sodium carbonate (48 mg, 0.45 mmol), (2-Methyl-2H-pyrazolo[3,4-b]pyridin-5-yl) boronic acid (41 mg, 0.23 mmol), Bis(di-tert-butyl (4-dimethylaminophenyl)phosphine)dichloropalladium (II) (4.9 mg, 6.9 μmol), and EtOH (1 mL). The vial headspace was flushed with Ar, and the vial was sealed with a pressure-relief septum cap. The reaction mixture was then stirred at 80° C. on an aluminum heating block under argon for 21 h, then concentrated at 40° C. under N₂. The residue was purified by silica gel chromatography using a gradient of 0-20% MeOH in CH₂Cl₂, to provide the title compound (60.2 mg, 100%) as a yellow oily solid. ES-MS m/z 354.2 (M+H).

### Preparation 264

### rac-(trans)-2-(2-Fluoro-4-(2-methyl-2H-pyrazolo[3,4-b]pyridin-5-yl)phenyl)-2-methylcyclopropane-1-carboxylic acid

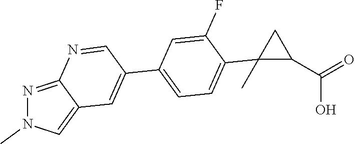 [SMILES: Cn1cc2cc(-c3ccc(C4(C)CC4C(=O)O)c(F)c3)cnc2n1]

A solution of rac-ethyl (trans)-2-(2-fluoro-4-(2-methyl-2H-pyrazolo[3,4-b]pyridin-5-yl)phenyl)-2-methylcyclopropane-1-carboxylate (60.2 mg, 91% wt, 155 μmol) in MeOH (1 mL) was treated with aq. lithium hydroxide (200 μL, 2 molar, 400 μmol). The reaction vial was flushed with Ar, sealed with a pressure-relief septum cap, and stirred at 65° C. for 18 h. The reaction mixture was cooled to RT and quenched with aq. HCl (300 μL, 1 molar, 300 μmol), then concentrated at 40° C. under N₂. The residue was further dried at 50° C. overnight in a vacuum oven to provide the title compound (assumed quantitative) as an off-white solid. ES-MS m z 326.2 (M+H).

### Preparation 265

### 1-(Methyl-d₃)-1H-pyrazole-4-carbaldehyde

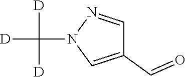 [SMILES: [2H]C([2H])([2H])n1cc(C=O)cn1]

1H-Pyrazole-4-carbaldehyde (10.1 g, 105 mmol) and cesium carbonate (33.6 g, 0.981 Eq, 103 mmol) were added to a 500 mL flask with a stir bar. DMF (200 mL) was added, and the flask was sealed and purged with N₂. Iodomethane-d₃ (7.73 mL, 124 mmol) was added. The reaction mixture was stirred at 60° C. overnight. The reaction was cooled down to RT, diluted with water (400 mL) and extracted with EtOAc (2×250 mL). The combined organic layers were washed with sat. aq. NaCl (3×50 mL), dried over Na₂SO₄ filtered and the filtrate was concentrated under reduced pressure. The residue was purified by silica gel chromatography using a gradient of 20%-80% EtOAc in hexane to obtain the title compound (4.06 g, 34% yield) as colorless liquid. 1H NMR (400 MHz, DMSO) δ 9.79 (s, 1H), 8.42 (d, J=0.7 Hz, 1H), 7.97 (d, J=0.7 Hz, 1H).

### Preparation 266

### Methyl (Z)-2-((tert-butoxycarbonyl)amino)-3-(1-(methyl-d₃)-1H-pyrazol-4-yl)acrylate

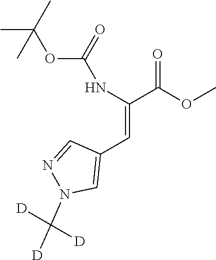 [SMILES: [2H]C([2H])([2H])n1cc(/C=C(\NC(=O)OC(C)(C)C)C(=O)OC)cn1]

Methyl 2-{[(tert-butoxy)carbonyl]amino}-2-(dimethoxyphosphoryl)acetate (12.0 g, 40.4 mmol) was added to a 100 mL flask containing 1-(methyl-d₃)-1H-pyrazole-4-carbaldehyde (4.0 g, 35 mmol) and purged with N₂. DCM (48 mL) was added, and the resulting clear solution was stirred at RT. DBU (6.1 g, 6.0 mL, 40 mmol) was added dropwise. The reaction mixture was stirred for 2 h at RT. The reaction mixture was diluted with DCM (30 mL), washed with water (50 mL), sat. aq. ammonium sulfate (50 mL), then sat. aq. K₂CO₃ (50 mL). The organic layer was dried over Na₂SO₄ filtered and the filtrate was concentrated under reduced pressure. The residue was purified by silica gel chromatography using a gradient of 50%-100% EtOAc in hexane to obtain the title compound (7.56 g, 75%) as a white solid, ES-MS m/z 285.4 (M+H)

### Preparation 267

### Methyl (R)-2-((tert-butoxycarbonyl)amino)-3-(1-(methyl-d₃)-1H-pyrazol-4-yl)propanoate

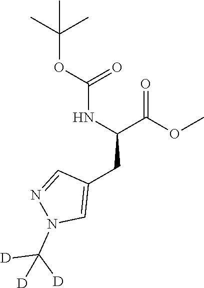 [SMILES: [2H]C([2H])([2H])n1cc(C[C@@H](NC(=O)OC(C)(C)C)C(=O)OC)cn1]

In a glove box under N₂, methyl (Z)-2-((tert-butoxycarbonyl)amino)-3-(1-(methyl-d₃)-1H-pyrazol-4-yl)acrylate (5.71 g, 20.1 mmol) and Rh-COD-[(R)-MaxPhos]-BF₄ (612 mg, 1.09 mmol) were added to a Parr stirred autoclave. 2,2,2-Trifluoroethanol (80 mL) was added. The autoclave was sealed and removed from the glove box. The autoclave was purged with H₂ and pressurized to 200 psi and stirred at 40° C. overnight. The mixture was cooled down to RT, the solvent was removed under reduced pressure. The residue was purified by silica gel chromatography using a gradient of 75%-100% EtOAc in hexane to obtain the title compound (4.82 g, 84%, 92% ee) as colorless, viscous liquid. Chiral analysis was carried out by using a Lux 5 μm i-Amylose-3, 4.6×100 mm, column with 15% IPA (0.2% IPAm)/CO₂ at 5 mL/min and detecting at 225 nm, Rt=1.24 min. ES-MS m/z 287.4 (M+H).

### Preparation 268

### tert-Butyl (R)-(1-((1-(5-bromo-3-(trifluoromethyl)-1H-pyrazol-1-yl)-2-methylpropan-2-yl)amino)-3-(1-(methyl-d₃)-1H-pyrazol-4-yl)-1-oxopropan-2-yl)carbamate

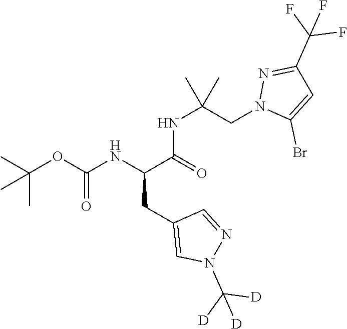 [SMILES: [2H]C([2H])([2H])n1cc(C[C@@H](NC(=O)OC(C)(C)C)C(=O)NC(C)(C)Cn2nc(C(F)(F)F)cc2Br)cn1]

Methyl (R)-2-((tert-butoxycarbonyl)amino)-3-(1-(methyl-d₃)-1H-pyrazol-4-yl)propanoate (1.29 g, 4.50 mmol) and a stirbar were charged to a 40 mL vial and tert-butyl alcohol (8 mL) was added. The mixture was sonicated until homogeneous, then water (1 mL) was added. The reaction mixture was stirred on an ice-water bath and lithium hydroxide (3 mL, 2 molar, 6 mmol) was added dropwise at 0° C. Upon complete addition, the reaction mixture was stirred at 0° C. for 2 h, quenched at 0° C. with slow addition of concentrated aq. HCl (200 μL, 2.40 mmol, 12M), and lyophilized. The resulting white foam was combined with 1-(5-bromo-3-(trifluoromethyl)-1H-pyrazol-1-yl)-2-methylpropan-2-amine hydrochloride (1.88 g, 5.83 mmol) and DMSO (5 mL), vortexed until homogeneous, and TEA (2 mL, 0.01 mol) was added, followed by HATU (2.5 g, 6.6 mmol). The reaction mixture was stirred at RT for 20 min, quenched with water (5 mL) and purified by reversed phase flash chromatography (275 g C18) using a gradient of 30-100% ACN in 0.1% aq. FA, to provide the title compound (3 g, 100%, 80% purity) as an oily solid. ES-MS m/z 540,542 (M+H).

### Preparation 269

### Methyl (R)-2-amino-3-(1-(difluoromethyl)-1H-pyrazol-4-yl)propanoate hydrochloride

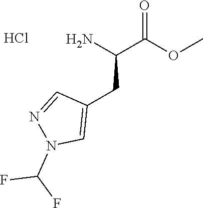

A solution of (R)-2-((tert-butoxycarbonyl)amino)-3-(1-(difluoromethyl)-1H-pyrazol-4-yl)propanoic acid (6.00 g, 19.5 mmol) in 2M HCl in MeOH (58.4 mL, 117 mmol) was stirred at RT for 22 h. The reaction mixture was then concentrated under reduced pressure to provide the title compound (5.01 g, 91%, 90% purity) as a white solid. ES-MS m/z 220 (M+H).

### Preparation 270

### Methyl-d₃ (R)-2-((tert-butoxycarbonyl)(methyl-d₃)amino)-3-(1-(difluoromethyl)-1H-pyrazol-4-yl)propanoate

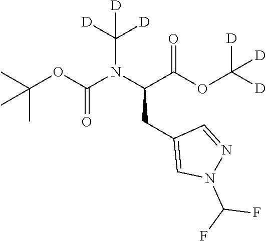 [SMILES: [2H]C([2H])([2H])OC(=O)[C@@H](Cc1cnn(C(F)F)c1)N(C(=O)OC(C)(C)C)C([2H])([2H])[2H]]

To a 40 mL vial was added a stirbar, (R)-2-((tert-butoxycarbonyl)amino)-3-(1-(difluoromethyl)-1H-pyrazol-4-yl)propanoic acid (1.533 g, 5.022 mmol), and silver oxide (4.2 g, 18 mmol). The solids were suspended in anhydrous DMF (10 mL) under argon and iodomethane-d₃ (3.0 g, 1.3 mL, 21 mmol) was added. The reaction vial was wrapped in aluminum foil and stirred at RT for 5 days, diluted to 40 mL total volume with MTBE, and filtered through diatomaceous earth, rinsing with additional MTBE (3×10 mL). The filtrate was washed with water (40 mL) and concentrated at 25° C. under reduced pressure. The residue was purified by silica gel chromatography using a gradient of 0-100% EtOAc in heptane (ELSD detection, TLC visualization with ninhydrin, product R/0.3 (30% EtOAc-heptane)) to provide the title compound (1.65 g, 96.8%) as a colorless oil. ES-MS m/z 340 (M+H).

### Preparation 271

### Lithium (R)-2-((tert-butoxycarbonyl)(methyl-d₃)amino)-3-(1-(difluoromethyl)-1H-pyrazol-4-yl)propanoate

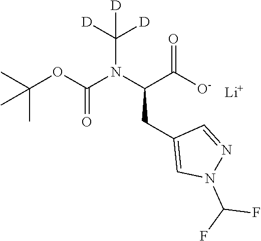 [SMILES: [2H]C([2H])([2H])N(C(=O)OC(C)(C)C)[C@H](Cc1cnn(C(F)F)c1)C(=O)[O-] ; [Li+]]

A 40 mL vial was charged with methyl-d₃ (R)-2-((tert-butoxycarbonyl)(methyl-d₃)amino)-3-(1-(difluoromethyl)-1H-pyrazol-4-yl)propanoate (1.65 g, 4.86 mmol) and a stirbar. 1,4-Dioxane (6 mL) and water (1 mL) were added and the reaction mixture was stirred at 0° C. under air to provide a homogeneous solution. Aq. lithium hydroxide (0.14 g, 3.0 mL, 2 molar, 6.0 mmol) was added dropwise, and the resulting cloudy solution was stirred at 0° C. for 5 min, then warmed to RT and stirred for 1 h. The reaction mixture was quenched with aq. HCl (1.5 mL, 1 molar, 1.5 mmol) and lyophilized to provide the title compound (assumed quantitative) as a white solid. ES-MS m/z 323 (M+H).

### Preparation 272

### tert-Butyl (1-((5-bromo-3-(trifluoromethyl)-1H-pyrazol-1-yl)methyl)cyclopropyl)carbamate

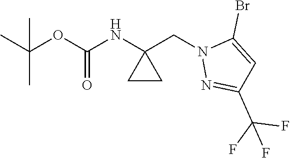 [SMILES: CC(C)(C)OC(=O)NC1(Cn2nc(C(F)(F)F)cc2Br)CC1]

A mixture of 3-bromo-5-(trifluoromethyl)-1H-pyrazole (6.11 g, 28.4 mmol), tert-butyl 6-oxa-5-thia-4-azaspiro[2.4]heptane-4-carboxylate 5,5-dioxide (4.72 g, 18.9 mmol) and Cs₂CO₃ (12.3 g, 37.9 mmol) in anhydrous DMA (50 mL) was stirred at 90° C. for 3 h under nitrogen. The reaction mixture was cooled to RT and poured into a stirring mixture of aq. 1 M hydrochloric acid (20 mL) and EtOAc (50 mL). Additional water and EtOAc were added until all solids were dissolved. The aq. layer was then acidified to a pH of ˜2-3 by addition of aq. 1 M hydrochloric acid (˜25-30 mL). The layers were separated, and the aq. layer was extracted with EtOAc (2× 50 mL). The organic layers were combined, dried over Na₂SO₄, filtered, and concentrated under reduced pressure. The residue was purified by silica gel chromatography using a gradient of 0-20% MTBE in cyclohexane to obtain the second eluting isomer as the title compound (2.08 g, 29%), white solid. ES-MS m/z 328,330 (M-tBu+H).

### Preparation 273

### 1-((5-Bromo-3-(trifluoromethyl)-1H-pyrazol-1-yl)methyl)cyclopropan-1-amine hydrochloride

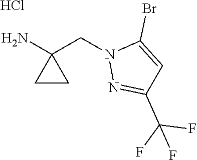 [SMILES: Cl ; NC1(Cn2nc(C(F)(F)F)cc2Br)CC1]

A solution of tert-butyl (1-((5-bromo-3-(trifluoromethyl)-1H-pyrazol-1-yl)methyl)cyclopropyl)carbamate (2.07 g, 5.39 mmol) in MeOH (5 mL) was treated with a 4.0 M solution of hydrogen chloride in 1,4-dioxane (15 mL, 60 mmol). The resulting mixture was stirred at RT for 2 h. The reaction mixture was then concentrated under reduced pressure to provide the title compound (1.73 g, 100%) as a white solid. ES-MS m/z 284,286 (M+H).

### Preparation 274

### tert-Butyl (R)-(1-((1-((5-bromo-3-(trifluoromethyl)-1H-pyrazol-1-yl)methyl)cyclopropyl)amino)-3-(1-(difluoromethyl)-1H-pyrazol-4-yl)-1-oxopropan-2-yl)carbamate

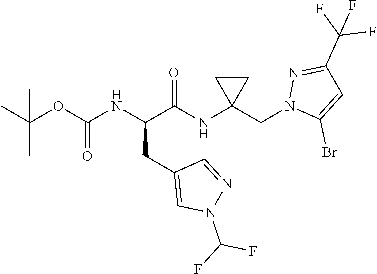 [SMILES: CC(C)(C)OC(=O)N[C@H](Cc1cnn(C(F)F)c1)C(=O)NC1(Cn2nc(C(F)(F)F)cc2Br)CC1]

A stirring mixture of (R)-2-((tert-butoxycarbonyl)amino)-3-(1-(difluoromethyl)-1H-pyrazol-4-yl)propanoic acid (1.74 g, 5.70 mmol), 1-((5-bromo-3-(trifluoromethyl)-1H-pyrazol-1-yl)methyl)cyclopropan-1-amine hydrochloride (1.73 g, 5.40 mmol), HATU (3.19 g, 8.39 mmol) in DMF (10 mL) was treated with DIEA (4.7 mL, 27 mmol). The resulting mixture was stirred at RT for 3 h. The reaction mixture was partitioned between EtOAc and saturated aq. sodium bicarbonate solution, diluting further with water until all solids dissolved. The layers were separated, and the aq. layer was extracted twice with EtOAc. The combined organic layers were washed with water then sat. aq. NaCl, dried over Na₂SO₄, filtered, and concentrated under reduced pressure. The residue was purified first on silica using a gradient of 0-20% 1:1 MTBE/DCM in DCM. Impure fractions were repurified by high pH reverse phase flash chromatography (40-65% ACN in 10 mM aq. NH₄HCO₃). Pure fractions from each purification were isolated to obtain the title compound (1.54 g, 50%) as a white solid. ES-MS m/z 515,517 (M-tBu+H).

### Preparation 275

### tert-Butyl (R)-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)(methyl-d₃)carbamate

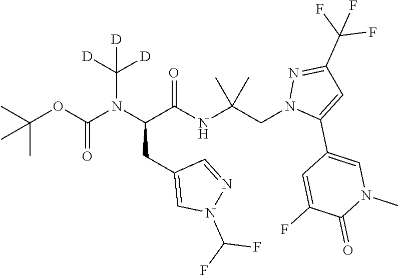 [SMILES: [2H]C([2H])([2H])N(C(=O)OC(C)(C)C)[C@H](Cc1cnn(C(F)F)c1)C(=O)NC(C)(C)Cn1nc(C(F)(F)F)cc1-c1cc(F)c(=O)n(C)c1]

A 20 mL vial was charged with a stirbar and 5-(1-(2-amino-2-methylpropyl)-3-(trifluoromethyl)-1H-pyrazol-5-yl)-3-fluoro-1-methylpyridin-2(1H)-one hydrochloride (608 mg, 1.65 mmol). Lithium (R)-2-((tert-butoxycarbonyl)(methyl-d₃)amino)-3-(1-(difluoromethyl)-1H-pyrazol-4-yl)propanoate (500 mg, 1.52 mmol) was added as a solution in DMSO (5 mL) and the reaction mixture was stirred at RT until homogeneous. TEA (800 μL, 5.74 mmol) was then added, resulting in a colorless biphasic mixture. HATU (778 mg, 2.05 mmol) was added and the reaction mixture was stirred at RT under air for 45 min, quenched with water (1 mL) and loaded directly onto diatomaceous earth with minimal MeOH. Reverse phase flash column chromatography (100 g C18), eluting with a gradient of 40-100% ACN in 0.1% aq. FA, provided the title compound (0.89 g, 92%) as a white solid. ES-MS m/z 637.4 (M+H).

### Preparation 276

### tert-Butyl (R)-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)carbamate

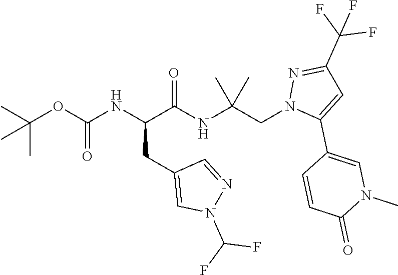 [SMILES: Cn1cc(-c2cc(C(F)(F)F)nn2CC(C)(C)NC(=O)[C@@H](Cc2cnn(C(F)F)c2)NC(=O)OC(C)(C)C)ccc1=O]

To a 1 L round bottom flask equipped with a stir bar was added 1-methyl-5-(4,4,5,5-tetramethyl-1,3,2-dioxaborolan-2-yl)pyridin-2(1H)-one (30.8 g 131 mmol), tert-butyl (R)-(1-((1-(5-bromo-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate (50.0 g, 87.2 mmol), Pd(dppf)-dichloride adduct (5.5 g, 6.7 mmol), and K₂CO₃ (36.2 g, 262 mmol) followed by 1,4-Dioxane (279 mL) and Water (69.8 mL). The mixture was vacuum degassed and purged with N₂ (3 x), heated to 90° C. for 1 hr. The mixture was diluted with DCM (500 mL) washed with water (1 L) The organic phase was dried over magnesium sulfate and concentrated under reduced pressure to yield a dark oil. The material was purified by silica gel chromatography using a gradient of 0-5% MeOH in EtOAc to provide the title compound (42 g, 80%) as a light tan foam solid. ES-MS m/z 602.4 (M+H).

The compounds in the following table were prepared as described in Preparation 276 using the appropriate aryl bromide and the appropriate boron ester or boronic acid. Reactants can be added in different orders or in differing equivalency, reaction times and temperatures may vary, and differing methods can be used to work up or purify the compounds, which would be apparent to one skilled in the art.

TABLE 20

| Preparation | Chemical Name | Structure | ES-MS m/z |
|---|---|---|---|
| 277 | tert-Butyl (R)-(1-((2-meth- yl-1-(5-(2-methylpyrim- idin-5-yl)-3-(trifluorometh- yl)-1H-pyrazol-1-yl)pro- pan-2-yl)amino)-3-(1- (methyl-d₃)-1H-pyrazol-4- yl)-1-oxopropan-2-yl)- carbamate | 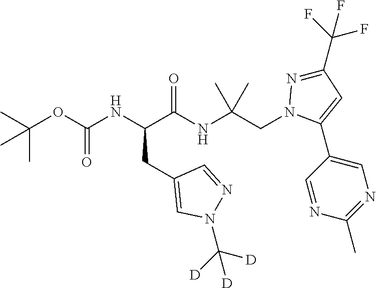 [SMILES: [2H]C([2H])([2H])n1cc(C[C@@H](NC(=O)OC(C)(C)C)C(=O)NC(C)(C)Cn2nc(C(F)(F)F)cc2-c2cnc(C)nc2)cn1] | 554 (M + H) |
| 278ª | tert-Butyl (R)-(3-(1- (difluoromethyl)-1H- pyrazol-4-yl)-1-((2-methyl- 1-(5-(2-methylpyrimidin-5- yl)-3-(trifluoromethyl)-1H- pyrazol-1-yl)propan-2-yl)- amino)-1-oxopropan-2-yl)- carbamate | 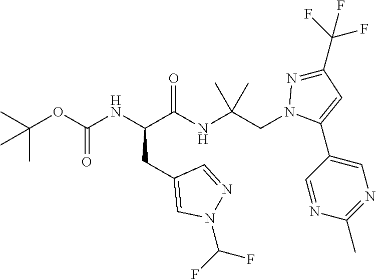 [SMILES: Cc1ncc(-c2cc(C(F)(F)F)nn2CC(C)(C)NC(=O)[C@@H](Cc2cnn(C(F)F)c2)NC(=O)OC(C)(C)C)cn1] | 531 (M − tBu + H) |
| 279^{b} | tert-Butyl (R)-(3-(1- (difluoromethyl)-1H- pyrazol-4-yl)-1-((1-((5-(5- fluoro-1-methyl-6-oxo- 1,6-dihydropyridin-3-yl)- 3-(trifluoromethyl)-1H- pyrazol-1-yl)methyl)- cyclopropyl)amino)-1- oxopropan-2-yl)carbamate | 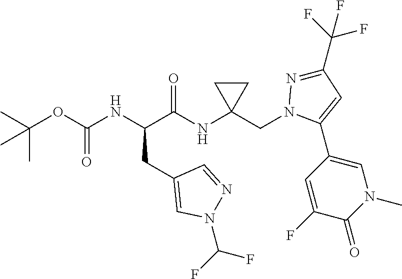 [SMILES: Cn1cc(-c2cc(C(F)(F)F)nn2CC2(NC(=O)[C@@H](Cc3cnn(C(F)F)c3)NC(=O)OC(C)(C)C)CC2)cc(F)c1=O] | 518 (M − tBu + H) |
| 280ª | tert-Butyl (R)-(3-(1-(di- fluoromethyl)-1H-pyrazol- 4-yl)-1-((2-methyl-1-(5-(2- cyclopropylpyrimidin-5- yl)-3-(trifluoromethyl)-1H- pyrazol-1-yl)-propan-2-yl)- amino)-1-oxopropan-2- yl)carbamate | 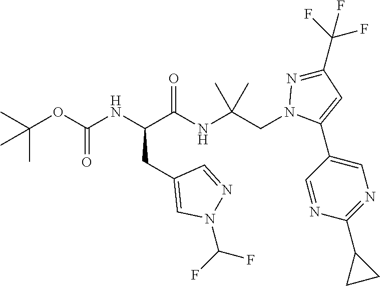 [SMILES: CC(C)(Cn1nc(C(F)(F)F)cc1-c1cnc(C2CC2)nc1)NC(=O)[C@@H](Cc1cnn(C(F)F)c1)NC(=O)OC(C)(C)C] | 557 (M − tBu + H) |
| 281ª | tert-Butyl (R)-(3-(1- (difluoromethyl)-1H-pyr- azol-4-yl)-1-((2-methyl-1- (5-(2-trifluoromethylpyr- imidin-5-yl)-3-(trifluoro- methyl)-1H-pyrazol-1- yl)propan-2-yl)amino)-1- oxopropan-2-yl)carbamate | 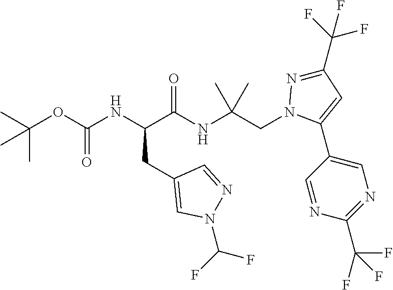 [SMILES: CC(C)(Cn1nc(C(F)(F)F)cc1-c1cnc(C(F)(F)F)nc1)NC(=O)[C@@H](Cc1cnn(C(F)F)c1)NC(=O)OC(C)(C)C] | 541 (M − Boc + H) |

^{a.}purified by silica gel chromatography eluting with EtOAc:EtOH (3:1) in heptane;
^{b.}purified by silica gel chromatography eluting with DCM:MeOH (4:1) (0-20%) in EtOAc.

### Preparation 282

### tert-Butyl (R)-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-thioxopropan-2-yl)carbamate

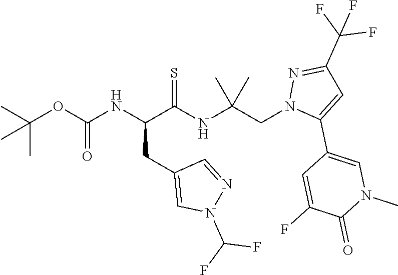 [SMILES: Cn1cc(-c2cc(C(F)(F)F)nn2CC(C)(C)NC(=S)[C@@H](Cc2cnn(C(F)F)c2)NC(=O)OC(C)(C)C)cc(F)c1=O]

A mixture of tert-butyl (R)-(3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)carbamate (500 mg, 807 μmol) and Lawesson's reagent (CAS #19172-47-5, 560 mg, 1.38 mmol) in THF (5 mL) was stirred at 70° C. overnight. The reaction mixture was cooled to RT and Lawesson's reagent (560 mg, 1.38 mmol) was added. The resulting mixture was stirred at 70° C. for 5 h. The reaction mixture was cooled to RT and concentrated under reduced pressure. The residue was purified by silica gel chromatography using a gradient of 30-100% EtOAc in cyclohexane to obtain the title compound (241 mg, 47%) as a yellow solid. ES-MS m/z 636.2 (M+H).

### Preparation 283

### (R)-2-amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide hydrochloride

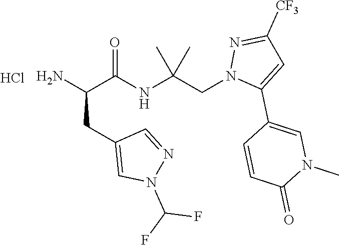 [SMILES: Cl ; Cn1cc(-c2cc(C(F)(F)F)nn2CC(C)(C)NC(=O)[C@H](N)Cc2cnn(C(F)F)c2)ccc1=O]

To a 1 L RBF with stir bar was added: tert-butyl (R)-(3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)carbamate (67.4 g, 112 mmol) and 1,4-dioxane (280 mL). The mixture was heated to 40° C. and monitored with an internal temperate probe. Hydrogen chloride 4M in 1,4-Dioxane (140 mL, 560 mmol) was added over 15 min and stirred overnight resulting in a thick oil in the bottom of the flask. The mixture was cooled to ambient temperature and 100 mL of MeOH was added to dissolve the thick oil. The solution was concentrated under reduced pressure at 40° C. This residue was then dissolved in MeOH (100 mL) and DCM (200 mL) and concentrated to provide the title compound (68.3, 110%) as a light tan foam solid. ES-MS (m z) 502.2 (M+H).

The compounds in the following table were prepared as described in Preparation 283 from the appropriate tert-butyl carbamate. Varying reaction times, equivalents of HCl can be used, which would be apparent to one skilled in the art. The deprotected amines were isolated as the corresponding hydrochloride salt. The compounds may have been used without determination of the number of equivalents of HCl.

TABLE 21

| | | | ES-MS |
|---|---|---|---|
| Preparation | Chemical Name | Structure | m/z |
| 284 | (R)-3-(1-(Difluoromethyl)- 1H-pyrazol-4-yl)-N-(1-(5- (5-fluoro-1-methyl-6-oxo- 1,6-dihydropyridin-3-yl)-3- (trifluoromethyl)-1H- pyrazol-1-yl)-2-methyl- propan-2-yl)-2-((methyl- d₃)amino)propanamide hydrochloride | 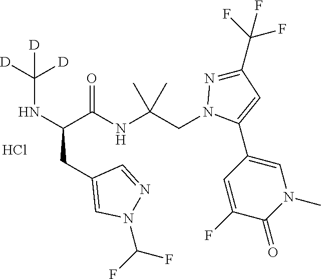 | 573 (M + H) |
| 285 | (R)-2-Amino-N-(2-methyl- 1-(5-(2-methylpyrimidin-5- yl)-3-(trifluoromethyl)-1H- pyrazol-1-yl)propan-2-yl)- 3-(1-(methyl-d₃)-1H- pyrazol-4-yl)propanamide hydrochloride | 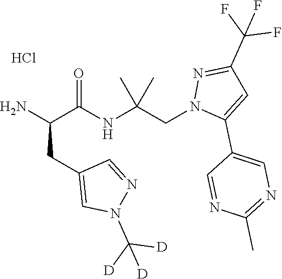 [SMILES: Cl ; [2H]C([2H])([2H])n1cc(C[C@@H](N)C(=O)NC(C)(C)Cn2nc(C(F)(F)F)cc2-c2cnc(C)nc2)cn1] | 454 (M + H) |
| 286ª | (R)-2-amino-3-(1- (difluoromethyl)-1H-pyr- azol-4-yl)-N-(1-(5-(5- fluoro-1-methyl-6-oxo- 1,6-dihydropyridin-3-yl)- 3-(trifluoromethyl)-1H- pyrazol-1-yl)-2-methyl- propan-2-yl)propanethio- amide hydrochloride | 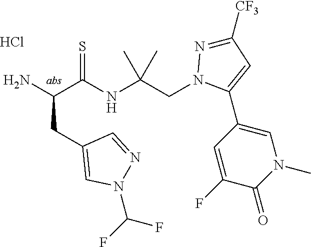 [SMILES: Cl ; Cn1cc(-c2cc(C(F)(F)F)nn2CC(C)(C)NC(=S)[C@H](N)Cc2cnn(C(F)F)c2)cc(F)c1=O] | 536 (M + H) |
| 287 | (R)-2-Amino-3-(1-(di- fluoromethyl)-1H-pyrazol- 4-yl)-N-(1-((5-(5-fluoro-1- methyl-6-oxo-1,6-dihydro- pyridin-3-yl)-3-(trifluoro- methyl)-1H-pyrazol-1-yl)- methyl)cyclopropyl)pro- panamide hydrochloride | 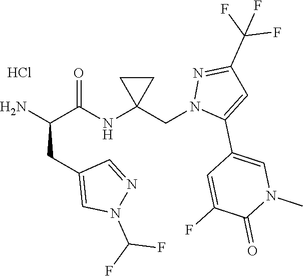 [SMILES: Cl ; Cn1cc(-c2cc(C(F)(F)F)nn2CC2(NC(=O)[C@H](N)Cc3cnn(C(F)F)c3)CC2)cc(F)c1=O] | 518 (M + H) |
| 288^{b} | (R)-2-Amino-3-(1-(di- fluoromethyl)-1H-pyrazol- 4-yl)-N-(2-methyl-1-(5-(2- methylpyrimidin-5-yl)-3- (trifluoromethyl)-1H-pyr- azol-1-yl)propan-2-yl)- propanamide hydrochloride | 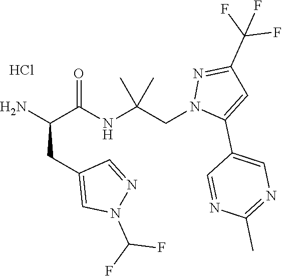 [SMILES: Cl ; Cc1ncc(-c2cc(C(F)(F)F)nn2CC(C)(C)NC(=O)[C@H](N)Cc2cnn(C(F)F)c2)cn1] | 487 (M + H) |
| 289^{b} | (R)-2-Amino-N-(1-(5-(2- cyclopropylpyrimidin-5- yl)-3-(trifluoromethyl)-1H- pyrazol-1-yl)-2-methylpro- pan-2-yl)-3-(1-(difluoro- methyl)-1H-pyrazol-4-yl)- propanamide hydrochloride | 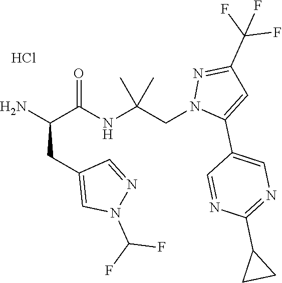 [SMILES: Cl ; CC(C)(Cn1nc(C(F)(F)F)cc1-c1cnc(C2CC2)nc1)NC(=O)[C@H](N)Cc1cnn(C(F)F)c1] | 513 (M + H) |
| 290^{b} | (R)-2-Amino-N-(1-(5-(2- trifluoromethylpyrimidin- 5-yl)-3-(trifluoromethyl)- 1H-pyrazol-1-yl)-2-meth- ylpropan-2-yl)-3-(1-(di- fluoromethyl)-1H-pyrazol- 4-yl)propanamide hydro- chloride | 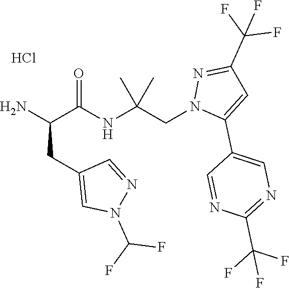 [SMILES: Cl ; CC(C)(Cn1nc(C(F)(F)F)cc1-c1cnc(C(F)(F)F)nc1)NC(=O)[C@H](N)Cc1cnn(C(F)F)c1] | 541 (M + H) |

^{a.}RT, 2.5 h, 10.5 eq HCl.
^{b.}RT, the crude reaction mixture was concentrated under reduced pressure to obtain the title compound.

### Preparation 291

### (6-Chloro-2-(trifluoromethyl)pyridin-3-yl)methanol

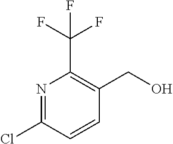 [SMILES: OCc1ccc(Cl)nc1C(F)(F)F]

6-Chloro-2-(trifluoromethyl) nicotinic acid (25 g, 0.11 mol) was dissolved in THF (100 mL) under Ar. Borane dimethylsulfide (19 mL, 0.22 mol) was added dropwise. The reaction was allowed to stir for 3 days, then was cooled to 0° C. and MeOH (30 mL, 0.74 mol) was added dropwise. Upon complete addition, the mixture was concentrated under reduced pressure and chased with additional MeOH (3×100 mL). The residue was re-suspended in MTBE (200 mL) and filtered. The filtrate was treated with 1M aq. NaOH (200 mL) and the aq. layer was extracted with MTBE (100 mL). The combined organics were dried over Na₂SO₄, filtered, and concentrated to obtain the title compound (25.7 g, 100%) as a light yellow-orange oil. ES-MS m/z 212.0/214.0 (M+H).

### Preparation 292

### (6-Chloro-2-(trifluoromethyl)pyridin-3-yl)methyl methanesulfonate

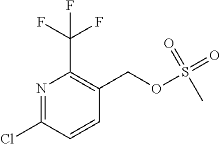 [SMILES: CS(=O)(=O)OCc1ccc(Cl)nc1C(F)(F)F]

(6-Chloro-2-(trifluoromethyl)pyridin-3-yl)methanol (24.7 g, 110 mmol) was dissolved in DCM (250 mL), and TEA (24 mL, 0.17 mol) was added under argon. The reaction mixture was cooled to 0° C. and methanesulfonyl chloride (10 mL, 0.13 mol) was added dropwise over 5 min. The reaction was allowed to stir for 20 min, then was diluted with water (200 mL) and allowed to warm to RT. The layers were separated, and the aq. layer was extracted with DCM (100 mL). The combined organics were concentrated under reduced pressure to obtain the title compound (32.72 g, 100%) as an orange-yellow oil. ES-MS m/z 290.0 (M+H).

### Preparation 293

### 1-((6-Chloro-2-(trifluoromethyl)pyridin-3-yl)methyl)tetrahydro-1H-thiophen-1-ium methanesulfonate

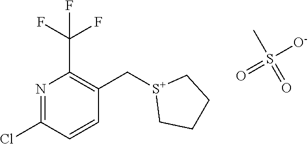

(6-Chloro-2-(trifluoromethyl)pyridin-3-yl)methyl methanesulfonate (31.3 g, 100 mmol) was dissolved in 2,2,2-trifluoroethanol (100 mL), and tetrahydrothiophene (10 mL, 0.11 mol) was added. The flask was sealed with a septum and the reaction mixture was mixed by swirling, then allowed to stand under air overnight. The reaction mixture was combined with 1.0 g scale reaction mixture and concentrated under reduced pressure to yield a thick oil. The residue was suspended in MTBE (500 mL) and filtered. The precipitate was washed with MTBE (3×200 mL) and air-dried on filter to obtain the title compound (34 g, 86%) as a white solid. ES-MS m/z 282 (M⁺).

### Preparation 294

### 1-((6-bromo-2-methylpyridin-3-yl)methyl)tetrahydro-1H-thiophen-1-ium tetrafluoroborate

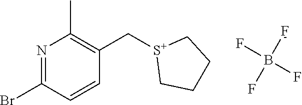

To 6-Bromo-3-(bromomethyl)-2-methylpyridine (2 g, 8 mmol) and tetrahydrothiophene (0.7 mL, 8 mmol) in ACN (10 mL) was added silver tetrafluoroborate (1 g, 8 mmol). The reaction was stirred for 6 h, then combined with a 1.5 g reaction mixture and partially concentrated (2 mL). The mixture was filtered over diatomaceous earth, concentrated to dryness, re-suspended in ACN (5 mL) and filtered. The filtrate was concentrated to afford the title compound (4.46 g, 50% purity, 45%) as a grey solid. ES/MS m/z 274 (M⁺).

### Preparation 295

### 1-(4-Bromo-2-chlorobenzyl)tetrahydro-1H-thiophen-1-ium bromide

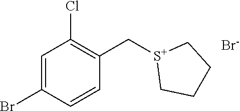 [SMILES: Clc1cc(Br)ccc1C[S+]1CCCC1 ; [Br-]]

4-Bromo-1-(bromomethyl)-2-chlorobenzene (10 g, 34 mmol), was dissolved in 2,2,2-trifluoroethanol (50 mL), and tetrahydrothiophene (4.0 mL, 45 mmol) was added. The resulting suspension was stirred at RT under air for 3 h then was concentrated at 25° C. under reduced pressure. The residue was resuspended in MTBE (100 mL) and filtered. The precipitate was rinsed with MTBE (2×50 mL) and air-dried to obtain the title compound (13.45 g, 100%) as a white powder. ES/MS m/z 291 (M⁺).

### Preparation 296

### 1-(4-Bromo-2-(difluoromethyl)benzyl)tetrahydro-1H-thiophen-1-ium bromide

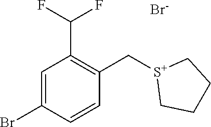 [SMILES: FC(F)c1cc(Br)ccc1C[S+]1CCCC1 ; [Br-]]

The title compound was prepared as described in Preparation 295, from 4-bromo-1-(bromomethyl)-2-(difluoromethyl)benzene, stirring overnight. ES-MS m/z 307 (M⁺).

### Preparation 297

### (trans)-1-(6-Chloro-2-(trifluoromethyl)pyridin-3-yl)-5-oxaspiro[2.4]heptan-4-one, isomer 1

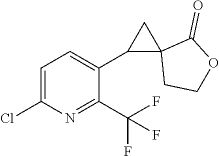 [SMILES: O=C1OCCC12CC2c1ccc(Cl)nc1C(F)(F)F]

1-((6-Chloro-2-(trifluoromethyl)pyridin-3-yl)methyl)tetrahydro-1H-thiophen-1-ium methanesulfonate (31.97 g, 83.77 mmol) and 3-methylenedihydrofuran-2(3H)-one (9.2 g, 94 mmol), were dissolved in DCM (120 mL) under Ar. The reaction mixture was cooled to 0° C. and LiHMDS (1 M in THF, 90 mL, 90 mmol) was added dropwise. The reaction mixture was stirred at 0° C. for 1 h then LiHMDS (1 M in THF, 12.5 mL, 12.5 mmol) was added dropwise. The mixture was stirred at 0° C. for an additional 30 min, then quenched with saturated aq. NH₄Cl (200 mL), allowed to warm to RT, and transferred to a 500 mL separatory funnel. The organic layer was collected, and the aq. layer was diluted with H₂O (50 mL) and extracted with additional DCM (2×100 mL). The combined extracts were concentrated under reduced pressure. The residue was suspended in MTBE (200 mL) with stirring for 5 min, then filtered, rinsing with MTBE (3× 20 mL). The filtrate was concentrated and dry-loaded onto silica and purified by column chromatography using a gradient of 30-100% EtOAc in heptane followed by chiral SFC purification (Chiralpak IG 30×250 mm×5 μM, 85 mL/min), eluting with 5% MeOH in 10 mM NH₄OAc in MeOH to afford the title compound (2 g, 7%) as the first eluting trans enantiomer.

### Preparation 298

### (1S,3R)-1-(4-bromo-2-chlorophenyl)-5-oxaspiro[2.4]heptan-4-one

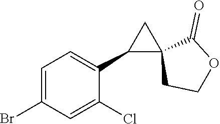 [SMILES: O=C1OCC[C@@]12C[C@@H]2c1ccc(Br)cc1Cl]

The title compound was prepared as described in Preparation 297 using 1-(4-bromo-2-chlorobenzyl)tetrahydro-1H-thiophen-1-ium bromide. Purified by silica using a gradient of 10-40% EtOAc/heptane followed by chiral SFC purification (Chiralpak IG 50 mm×250× 5 μm, 80 mL/min), eluting with MeOH to afford the title compound (1.43 g, 14%) as the first eluting enantiomer. ES-MS m/z 301.0, 303.0 (M+H).

### Preparation 299

### rac-(trans)-1-(4-Bromo-2-(difluoromethyl)phenyl)-5-oxaspiro[2.4]heptan-4-one

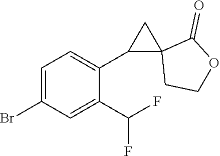 [SMILES: O=C1OCCC12CC2c1ccc(Br)cc1C(F)F]

3-Methylenedihydrofuran-2(3H)-one (0.50 g, 5.1 mmol) and 1-(4-bromo-2-(difluoromethyl)benzyl)tetrahydro-1H-thiophen-1-ium bromide (1.4 g, 3.6 mmol) were combined in DCM (12 mL) and cooled to 0° C. LiHMDS (1 M in toluene, 4.7 mL, 4.7 mmol) was added into flask dropwise. The reaction was stirred at 0° C. for 1 h, then LiHMDS (1 M in toluene, 2.35 mL, 2.35 mmol) was added and the reaction was allowed to stir for 15 min. The reaction mixture was then diluted with water and DCM and warmed to RT. The aq. layer was extracted with DCM (3×) and the organic layers were combined, dried over sodium sulfate, and concentrated. The reaction mixture was combined with 500 mg scale reaction mixture, and purified by UPLC CSH (XSelect, C18, 50 mm×250 mm×5 μm, 100 mL/min) using a gradient of 40-95% ACN/water (w/10 mM NH₄OAc) to obtain the title compound (721 mg, 48%) as a white solid. ES-MS m/z 317.0/319.0 (M+H).

### Preparation 300

### Rac-(trans)-1-(6-Bromo-2-methylpyridin-3-yl)-5-oxaspiro[2.4]heptan-4-one

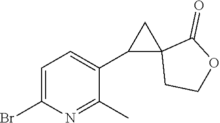 [SMILES: Cc1nc(Br)ccc1C1CC12CCOC2=O]

The title compound was prepared as described in Preparation 299 using tetrafluoro-24-borane, 1-((6-bromo-2-methylpyridin-3-yl)methyl)tetrahydro-1H-thiophen-1-ium salt. The product was purified by silica using a gradient of 0-40% MeOH in DCM to obtain the title compound (430 mg, 32%). ES-MS m/z 282,284 (M+H).

### Preparation 301

### Methyl (R)-2-((1R,2R)-1-(2-chloroethyl)-2-(4-(2-methyl-2H-pyrazolo[3,4-b]pyridin-5-yl)phenyl)cyclopropane-1-carboxamido)-3-(1-(difluoromethyl)-1H-pyrazol-4-yl)propanoate

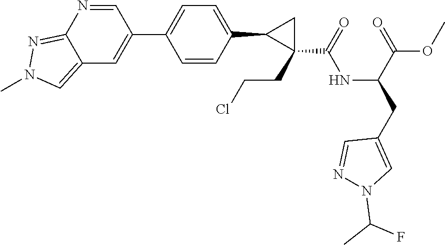

To a mixture of methyl (R)-2-amino-3-(1-(difluoromethyl)-1H-pyrazol-4-yl)propanoate hydrochloride (5.4 g, 90 wt %, 19 mmol) and TEA (26 mL, 0.19 mmol) in THF (120 mL) and DMA (60 mL) was added (1R,2R)-1-(2-chloroethyl)-2-(4-(2-methyl-2H-pyrazolo[3,4-b]pyridin-5-yl)phenyl)cyclopropane-1-carbonyl chloride (17.2 g, 47 wt %, 21.6 mmol) at 0° C., then the reaction mixture was stirred at 20° C. for 2 h. The reaction mixture was poured into water (150 mL) and the mixture was extracted with EtOAc (2×150 mL). The combined organic layers were washed with sat. aq. NaCl (2× 200 mL), dried over sodium sulfate, filtered, and the filtrate concentrated under reduced pressure. The residue was triturated with EtOAc (100 mL) at RT for 1 h. The mixture was filtered, and the filtrate concentrated under reduced pressure to provide residue. The crude material was purified by silica gel chromatography eluting with 0-3% MeOH/DCM to obtain the title compound (8.3 g, 74%) as a light-yellow solid. ES-MS m/z 557 (M+H).

### Preparation 302

### Methyl (R)-3-(1-(difluoromethyl)-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanoate

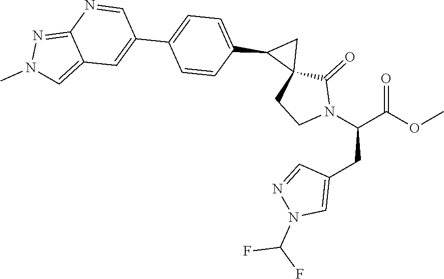

A mixture of methyl (R)-2-((1R,2R)-1-(2-chloroethyl)-2-(4-(2-methyl-2H-pyrazolo[3,4-b]pyridin-5-yl)phenyl)cyclopropane-1-carboxamido)-3-(1-(difluoromethyl)-1H-pyrazol-4-yl)propanoate (8.3 g, 14 mmol) and cesium carbonate (9.2 g, 28 mmol) in THF (150 mL) was stirred at 50° C. for 16 h. The reaction mixture was cooled to RT then was poured into water (200 mL). The mixture was extracted with EtOAc (2×200 mL). The combined organic layers were washed with sat. aq. NaCl (2×300 mL), dried over Na₂SO₄, filtered, and the filtrate concentrated under reduced pressure. The residue was purified by silica gel chromatography eluting with 0-5% MeOH/DCM to obtain the title compound (6.2 g, 78%) as a yellow solid. ES-MS m/z 521 (M+H).

### Preparation 303

### (R)-3-(1-(Difluoromethyl)-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanoic acid

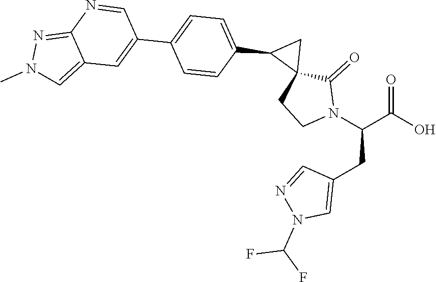 [SMILES: Cn1cc2cc(-c3ccc([C@H]4C[C@@]45CCN([C@H](Cc4cnn(C(F)F)c4)C(=O)O)C5=O)cc3)cnc2n1]

To a solution of methyl (R)-3-(1-(difluoromethyl)-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanoate (3.6 g, 6.6 mmol) in t-BuOH (32 mL) and water (16 mL) was added a 1 M aq. solution of lithium hydroxide (20 mL, 20 mmol) at 0° C. The reaction mixture was stirred at 0° C. for 3 h. The reaction mixture was then concentrated at 20° C. to remove t-BuOH providing a basic aq. solution. The aq. mixture was treated with strongly acidic styrene cation exchange resin (18.2 g), then the resin was filtered and rinsed with water (300 mL). The filtrate was lyophilized to dryness to provide the title compound (2.42 g, 71%) as a yellow solid. ES-MS m/z 507 (M+H).

### Preparation 304

### Methyl (R)-2-((1R,2S)-2-(4-bromo-2-fluorophenyl)-1-(2-chloroethyl)cyclopropane-1-carboxamido)-3-(1-(difluoromethyl)-1H-pyrazol-4-yl)propanoate

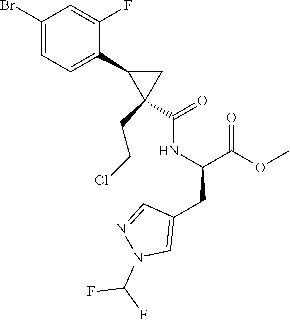 [SMILES: COC(=O)[C@@H](Cc1cnn(C(F)F)c1)NC(=O)[C@@]1(CCCl)C[C@@H]1c1ccc(Br)cc1F]

To a solution of (1S,3R)-1-(4-bromo-2-fluorophenyl)-5-oxaspiro[2.4]heptan-4-one (4.04 g, 13.3 mmol) and benzyl (triethyl) ammonium chloride (6.07 g, 26.6 mmol) in DCE (80 mL) were added boron trifluoride etherate (3.86 g, 3.41 mL, 26.6 mmol) and thionyl chloride (6.34 g, 3.92 mL, 53.3 mmol). The mixture was purged with N₂ 3 times, then stirred at 80° C. for 16 h under nitrogen. The mixture was concentrated under reduced pressure to give (1R,2R)-2-(4-bromo-2-fluorophenyl)-1-(2-chloroethyl)cyclopropane-1-carbonyl chloride (10.2 g, 45 wt % purity).

To a mixture of methyl (R)-2-amino-3-(1-(difluoromethyl)-1H-pyrazol-4-yl)propanoate hydrochloride (3.6 g, 13 mmol) and TEA (19 mL, 0.13 mmol) in THF (120 mL) and DMA (60 mL) was added a solution of (1R,2R)-2-(4-bromo-2-fluorophenyl)-1-(2-chloroethyl)cyclopropane-1-carbonyl chloride (10.2 g, 45 wt %, 13.5 mmol) in THF (80 mL) at 0° C., then the reaction mixture was stirred at 20° C. for 2 h. The reaction mixture was poured into water (150 mL) and the mixture was extracted with EtOAc (2×150 mL). The combined organic layers were washed with sat. aq. NaCl (2×100 mL), dried over sodium sulfate, filtered, and the filtrate concentrated under reduced pressure. The residue was purified by silica gel chromatography eluting with 0-40% EtOAc/PE to obtain the title compound (7.8 g, 95%, 85% purity) as a yellow oil. ES-MS m/z 522,524 (M+H).

### Preparation 305

### Methyl (R)-2-((1S,3R)-1-(4-bromo-2-fluorophenyl)-4-oxo-5-azaspiro[2.4]heptan-5-yl)-3-(1-(difluoromethyl)-1H-pyrazol-4-yl)propanoate

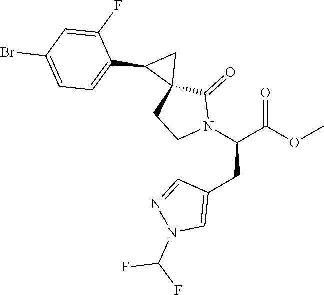 [SMILES: COC(=O)[C@@H](Cc1cnn(C(F)F)c1)N1CC[C@@]2(C[C@@H]2c2ccc(Br)cc2F)C1=O]

A mixture of methyl (R)-2-((1R,2S)-2-(4-bromo-2-fluorophenyl)-1-(2-chloroethyl)cyclopropane-1-carboxamido)-3-(1-(difluoromethyl)-1H-pyrazol-4-yl)propanoate (7.8 g, 13 mmol, 85 wt %) and cesium carbonate (5.0 g, 15 mmol) in THF (120 mL) was stirred at 50° C. for 24 h, then at RT for 48 h. The reaction mixture was poured into water (100 mL). The mixture was extracted with EtOAc (2×150 mL). The combined organic layers were washed with sat. aq. NaCl (2× 800 mL), dried over Na₂SO₄, filtered, and the filtrate concentrated under reduced pressure. The residue was purified by silica gel chromatography eluting with 0-35% EtOAc in PE to obtain the title compound (4.1 g, 63%, 65:35 d.r.) as a yellow oil. ES-MS m/z 486,488 (M+H).

### Preparation 306

### Methyl (R)-3-(1-(difluoromethyl)-1H-pyrazol-4-yl)-2-((1S,3R)-1-(2-fluoro-4-(2-methyl-2H-pyrazolo[3,4-b]pyridin-5-yl)phenyl)-4-oxo-5-azaspiro[2.4]heptan-5-yl)propanoate

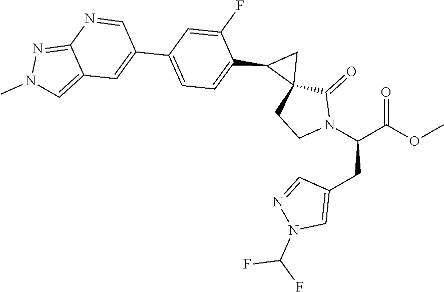

To a mixture of methyl (R)-2-((1S,3R)-1-(4-bromo-2-fluorophenyl)-4-oxo-5-azaspiro[2.4]heptan-5-yl)-3-(1-(difluoromethyl)-1H-pyrazol-4-yl)propanoate (4.12 g, 65:35 d.r., 7.96 mmol), (2-methyl-2H-pyrazolo[3,4-b]pyridin-5-yl) boronic acid (1.48 g, 8.36 mmol) and sodium carbonate (1.69 g, 15.9 mmol) in 1,4-dioxane (60 mL) and water (6 mL) was added XPhos Pd G3 (1.35 g, 1.59 mmol) at RT under nitrogen. The mixture was purged with nitrogen (3×), then the reaction mixture was stirred at 90° C. for 1 h. The reaction mixture was cooled to RT then poured into water (150 mL). The mixture was extracted with EtOAc (2×150 mL). The combined organic layers were washed with sat. aq. NaCl (2×150 mL), dried over Na₂SO₄, filtered, and the filtrate concentrated under reduced pressure. The residue was purified by silica gel chromatography eluting with 0-5% MeOH/DCM to obtain the title compound (4.35 g, 65:35 d.r., 92%) as a yellow solid. ES-MS m/z 539 (M+H).

### Preparation 307

### (R)-3-(1-(Difluoromethyl)-1H-pyrazol-4-yl)-2-((1S,3R)-1-(2-fluoro-4-(2-methyl-2H-pyrazolo[3,4-b]pyridin-5-yl)phenyl)-4-oxo-5-azaspiro[2.4]heptan-5-yl)propanoic acid

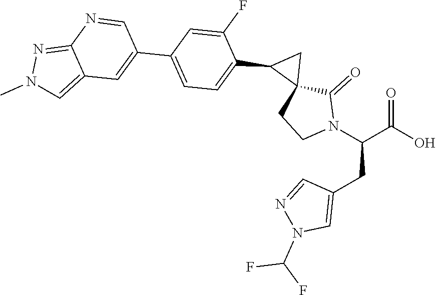 [SMILES: Cn1cc2cc(-c3ccc([C@H]4C[C@@]45CCN([C@H](Cc4cnn(C(F)F)c4)C(=O)O)C5=O)c(F)c3)cnc2n1]

To a solution of methyl (R)-3-(1-(difluoromethyl)-1H-pyrazol-4-yl)-2-((1S,3R)-1-(2-fluoro-4-(2-methyl-2H-pyrazolo[3,4-b]pyridin-5-yl)phenyl)-4-oxo-5-azaspiro[2.4]heptan-5-yl)propanoate (4.35 g, 2:1 d.r., 7.35 mmol) in t-BuOH (60 mL) and water (30 mL) was added a 1 M aq. solution of lithium hydroxide (11.0 mL, 11.0 mmol) at 0° C. The reaction mixture was stirred at 0° C. for 3 h. The reaction mixture was adjusted to PH ˜7 with 1 M aq. hydrochloric acid solution then concentrated under reduced pressure. The residue was purified by SFC (Daicel IM 250×30 mm, 10 μm, 40% CO₂ in ACN/EtOH containing 0.1% NH₄OH) to provide the second-eluting isomer as the title compound (2.57 g, 66%) as a white solid. ES-MS m/z 525 (M+H).

### Preparation 308

### (1R,2S)-2-(4-Bromo-2-fluorophenyl)-1-(2-chloroethyl)-N—((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)cyclopropane-1-carboxamide

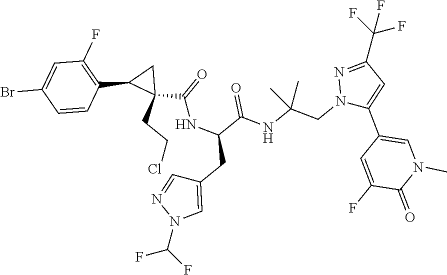 [SMILES: Cn1cc(-c2cc(C(F)(F)F)nn2CC(C)(C)NC(=O)[C@@H](Cc2cnn(C(F)F)c2)NC(=O)[C@@]2(CCCl)C[C@@H]2c2ccc(Br)cc2F)cc(F)c1=O]

A mixture of (1S,3R)-1-(4-Bromo-2-fluorophenyl)-5-oxaspiro[2.4]heptan-4-one (1.49 g, 5.23 mmol) and benzyltriethylammonium chloride (2.38 g, 10.4 mmol) in DCE (9 mL) was treated with SOCl₂ (1.55 mL, 21.2 mmol) followed by BF₃·Et₂O (1.3 mL, 11.0 mmol). After stirring at 80° C. overnight, the reaction mixture was cooled to RT and concentrated under reduced pressure. DCE (5 mL) was added, and the mixture was concentrated under reduced pressure to obtain a quantitative yield of (trans)-2-(4-bromo-2-fluorophenyl)-1-(2-chloroethyl)cyclopropane-1-carbonyl chloride, Isomer 1 as a yellow sticky solid. In a separate vessel, a mixture of (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide hydrochloride (3.10 g, 5.58 mmol) in DCM (20 mL) was stirred at 0° C. and treated with TEA (5.2 mL, 37.0 mmol). A suspension of (trans)-2-(4-bromo-2-fluorophenyl)-1-(2-chloroethyl)cyclopropane-1-carbonyl chloride (Isomer 1, 5.24 mmol) in DCM (5 mL) was added dropwise. The cooling bath was removed, and the reaction mixture was stirred at RT for 1 h. TEA (1.5 mL) was added dropwise and the resulting mixture was stirred at RT for 1 h. EtOAc (500 mL) and cold water (500 mL) were added, and the layers were separated. The aq. layer was extracted with EtOAc (200 mL), and the organic layers were combined, washed with water three times and then washed with sat. aq. NaCl, dried over Na₂SO₄, filtered, and concentrated under reduced pressure to obtain a quantitative yield of the title compound as a light-brown solid. ES-MS m/z 822,824 (M+H).

The compounds in the following table were prepared as described in Preparation 308 using the appropriate lactone and appropriate amine or amine salt. DIEA is a suitable replacement for TEA. Slight variations to the reaction conditions, work-up procedure and purification methods would be apparent to one skilled in the art.

TABLE 22

| | | | ES-MS |
|---|---|---|---|
| Preparation | Chemical Name | Structure | m/z |
| 309 | (trans)-2-(6-Chloro-2- (trifluoromethyl)pyr- idin-3-yl)-1-(2-chloro- ethyl)-N-((R)-3-(1- (difluoromethyl)-1H- pyrazol-4-yl)-1-((2- methyl-1-(5-(2-meth- ylpyrimidin-5-yl)-3- (trifluoromethyl)-1H- pyrazol-1-yl)propan- 2-yl)amino)-1-oxopro- pan-2-yl)cyclopropane- 1-carboxamide, isomer 1 | 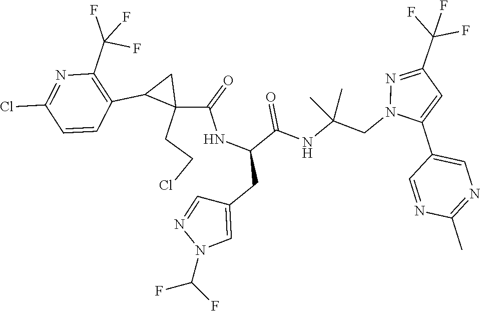 [SMILES: Cc1ncc(-c2cc(C(F)(F)F)nn2CC(C)(C)NC(=O)[C@@H](Cc2cnn(C(F)F)c2)NC(=O)C2(CCCl)CC2c2ccc(Cl)nc2C(F)(F)F)cn1] | 796 (M + H) |
| 310 | (1R,2S)-2-(4-Bromo-2- chlorophenyl)-1-(2- chloroethyl)-N-((R)-1- ((2-methyl-1-(5-(2- methylpyrimidin-5-yl)- 3-(trifluoromethyl)- 1H-pyrazol-1-yl)pro- pan-2-yl)amino)-3-(1- (methyl-d₃)-1H-pyr- azol-4-yl)-1-oxopro- pan-2-yl)cyclopro- pane-1-carboxamide | 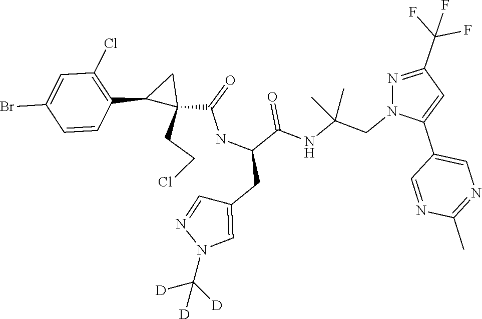 [SMILES: [2H]C([2H])([2H])n1cc(C[C@@H](NC(=O)[C@@]2(CCCl)C[C@@H]2c2ccc(Br)cc2Cl)C(=O)NC(C)(C)Cn2nc(C(F)(F)F)cc2-c2cnc(C)nc2)cn1] | 774 (M + H) |
| 311 | (trans)-2-(6-Chloro-2- (trifluoromethyl)pyr- idin-3-yl)-1-(2-chloro- ethyl)-N-((R)-3-(1- (difluoromethyl)-1H- pyrazol-4-yl)-1-((2- methyl-1-(5-(1-meth- yl-6-oxo-1,6-dihydro- pyridin-3-yl)-(tri- fluoromethyl)-1H- pyrazol-1-yl)propan- 2-yl)amino)-1-oxopro- pan-2-yl)cyclopro- pane-1-carboxamide, isomer 1 | 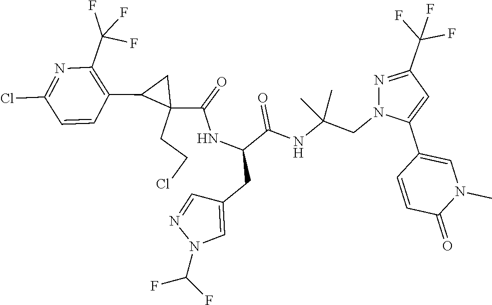 [SMILES: Cn1cc(-c2cc(C(F)(F)F)nn2CC(C)(C)NC(=O)[C@@H](Cc2cnn(C(F)F)c2)NC(=O)C2(CCCl)CC2c2ccc(Cl)nc2C(F)(F)F)ccc1=O] | 811 (M + H) |
| 312 | (trans)-2-(6-Chloro-2- (trifluoromethyl)pyr- idin-3-yl)-1-(2-chloro- ethyl)-N-((R)-1-((2- methyl-1-(5-(2-meth- ylpyrimidin-5-yl)-3- (trifluoromethyl)-1H- pyrazol-1-yl)propan-2- yl)amino)-3-(1-(meth- yl-d₃)-1H-pyrazol-4- yl)-1-oxopropan-2-yl)- cyclopropane-1-carbox- amide, isomer 1 | 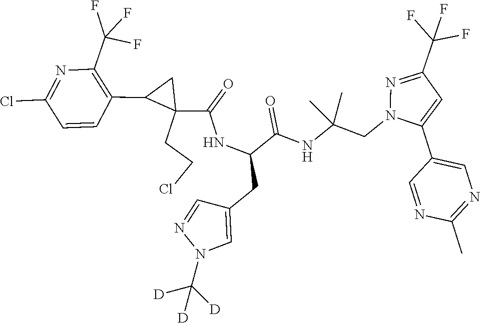 [SMILES: [2H]C([2H])([2H])n1cc(C[C@@H](NC(=O)C2(CCCl)CC2c2ccc(Cl)nc2C(F)(F)F)C(=O)NC(C)(C)Cn2nc(C(F)(F)F)cc2-c2cnc(C)nc2)cn1] | 763 (M + H) |
| 313ª | (1R,2R)-2-(4-Bromo- phenyl)-1-(2-chloro- ethyl)-N-((R)-1-((1- (5-(2-cyclopropylpyr- imidin-5-yl)-3-tri- fluoromethyl)-1H-pyr- azol-1-yl)-2-methyl- propan-2-yl)amino)- 3-(1-(difluoromethyl)- 1H-pyrazol-4-yl)-1- oxopropan-2-yl)cyclo- propane-1-carboxamide | 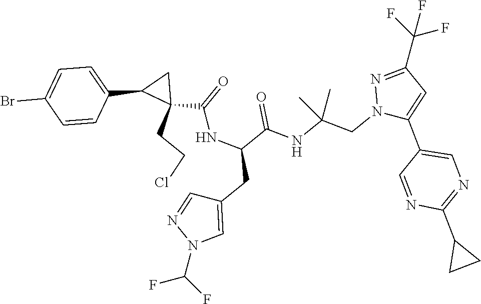 [SMILES: CC(C)(Cn1nc(C(F)(F)F)cc1-c1cnc(C2CC2)nc1)NC(=O)[C@@H](Cc1cnn(C(F)F)c1)NC(=O)[C@@]1(CCCl)C[C@@H]1c1ccc(Br)cc1] | 795,797 (M + H) |
| 314ª | (1R,2R)-2-(4-Bromo- phenyl)-1-(2-chloro- ethyl)-N-((R)-1-((1- (5-(2-trifluoromethyl- pyrimidin-5-yl)-3-(tri- fluoromethyl)-1H- pyrazol-1-yl)-2-meth- ylpropan-2-yl)amino)- 3-(1-(difluoromethyl)- 1H-pyrazol-4-yl)-1- oxopropan-2-yl)cyclo- propane-1-carboxamide | 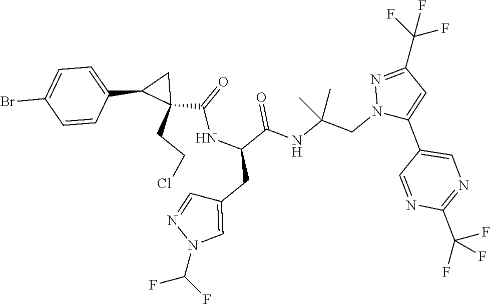 [SMILES: CC(C)(Cn1nc(C(F)(F)F)cc1-c1cnc(C(F)(F)F)nc1)NC(=O)[C@@H](Cc1cnn(C(F)F)c1)NC(=O)[C@@]1(CCCl)C[C@@H]1c1ccc(Br)cc1] | 825,827 (M + H) |
| 315^{b} | (trans)-2-(6-Chloro-2- methylpyridin-3-yl)-1- (2-chloroethyl)-N-((R)- 3-(1-(difluoromethyl)- 1H-pyrazol-4-yl)-1-((2- methyl-1-(5-(2-meth- ylpyrimidin-5-yl)-3- (trifluoromethyl)-1H- pyrazol-1-yl)propan-2- yl)amino)-1-oxopro- pan-2-yl)cyclopropane- 1-carboxamide | 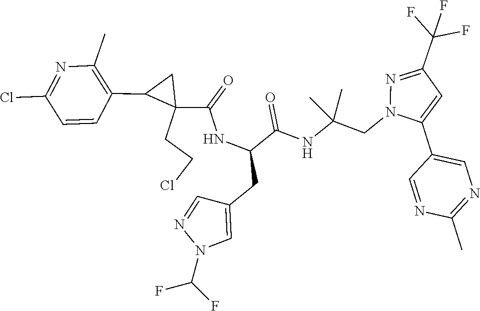 [SMILES: Cc1ncc(-c2cc(C(F)(F)F)nn2CC(C)(C)NC(=O)[C@@H](Cc2cnn(C(F)F)c2)NC(=O)C2(CCCl)CC2c2ccc(Cl)nc2C)cn1] | 742,744 (M + H) |
| 316^{c} | (trans)-2-(4-bromo-2- (difluoromethyl)phen- yl)-1-(2-chloroethyl)- N-((R)-3-(1-(difluoro- methyl)-1H-pyrazol-4- yl)-1-((1-(5-(5-fluoro- 1-methyl-6-oxo-1,6- dihydropyridin-3-yl)- 3-(trifluoromethyl)- 1H-pyrazol-1-yl)-2- methylpropan-2-yl)- amino)-1-oxopropan- 2-yl)cyclopropane-1- carboxamide | 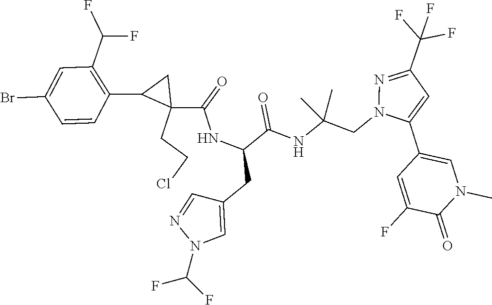 [SMILES: Cn1cc(-c2cc(C(F)(F)F)nn2CC(C)(C)NC(=O)[C@@H](Cc2cnn(C(F)F)c2)NC(=O)C2(CCCl)CC2c2ccc(Br)cc2C(F)F)cc(F)c1=O] | 854,856 (M + H) |
| 317^{d} | (1R,2R)-2-(4-bromo- phenyl)-1-(2-chloro- ethyl)-N-((R)-3-(1- (difluoromethyl)-1H- pyrazol-4-yl)-1-((2- methyl-1-(5-(1-meth- yl-6-oxo-1,6-dihydro- pyridin-3-yl)-3-(tri- fluoromethyl)-1H-pyr- azol-1-yl)propan-2- yl)amino)-1-oxopro- pan-2-yl)cyclopropane- 1-carboxamide | 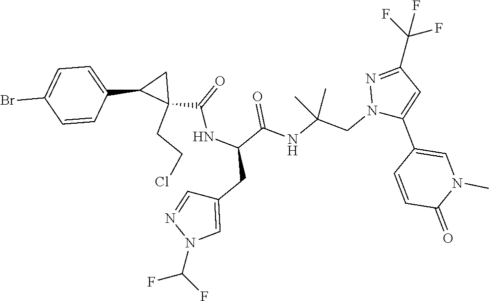 [SMILES: Cn1cc(-c2cc(C(F)(F)F)nn2CC(C)(C)NC(=O)[C@@H](Cc2cnn(C(F)F)c2)NC(=O)[C@@]2(CCCl)C[C@@H]2c2ccc(Br)cc2)ccc1=O] | 786,788 (M + H) |

^{a.}No work up. The reaction mixture was concentrated under reduced pressure and the residue was purified by silica gel chromatography using a gradient of 0-80% EtOAc in heptane;
^{b.}synthesized from rac-(trans)-1-(6-bromo-2-methylpyridin-3-yl)-5-oxaspiro[2.4]heptan-4-one as described above. Bromopyridyl was also converted to the chloropyridyl under the reaction conditions. Purified by silica gel chromatography, eluting with 3:1 EtOAc:EtOH in heptane;
^{c.}No work up. The reaction mixture was concentrated under reduced pressure and the residue was purified by silica gel chromatography using a gradient of 0-100% EtOAc in EtOH (3/1) in heptane;
^{d.}No work up. The reaction mixture was concentrated under reduced pressure and the residue was purified by silica gel chromatography using a gradient of 0-100% EtOAc in EtOH (3/1) in DCM.

### Preparation 318

### (2R)-2-(1S,3R)-(1-(4-Bromo-2-fluorophenyl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide

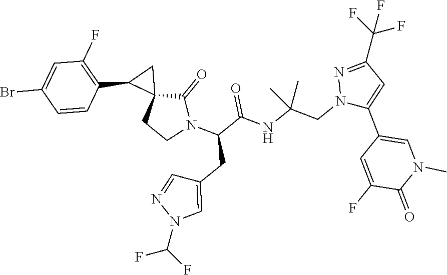 [SMILES: Cn1cc(-c2cc(C(F)(F)F)nn2CC(C)(C)NC(=O)[C@@H](Cc2cnn(C(F)F)c2)N2CC[C@@]3(C[C@@H]3c3ccc(Br)cc3F)C2=O)cc(F)c1=O]

A mixture of (1R,2S)-2-(4-bromo-2-fluorophenyl)-1-(2-chloroethyl)-N—((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)cyclopropane-1-carboxamide (4.57 g, 5.28 mmol) and Cs₂CO₃ (3.50 g, 11 mmol) in ACN (15 mL) was stirred overnight at RT. The reaction mixture was diluted ACN, filtered and washed with ACN. The filtrate was concentrated under reduced pressure. The residue was purified by reversed phase prep-HPLC (Luna® C18, 50×250 mm, 10 μm, 120 mL/min) using a gradient of 35-65% ACN in 10 mM aq. NH₄HCO₃, pH=10 (with 5% MeOH) over 22.5 min to obtain the title compound (2.94 g, 71%) as a pink foam. ES-MS m/z 786,788 (M+H).

The compounds in the following table were prepared as described in Preparation 318. NMP and THF are suitable replacements for ACN. Reaction times and temperatures may vary, and differing methods can be used to work up or purify the compounds (normal phase, or high, neutral or low pH reversed phase). Such variances would be apparent to one skilled in the art.

TABLE 23

| | | | ES-MS |
|---|---|---|---|
| Preparation | Chemical Name | Structure | m/z |
| 319ª | (R)-2-((trans)-1-(6-chloro-2- (trifluoromethyl)pyridin-3- yl)-4-oxo-5- azaspiro[2.4]heptan-5-yl)-3- (1-(difluoromethyl)-1H- pyrazol-4-yl)-N-(2-methyl-1- (5-(2-methylpyrimidin-5-yl)- 3-(trifluoromethyl)-1H- pyrazol-1-yl)propan-2- yl)propanamide, isomer 1 | 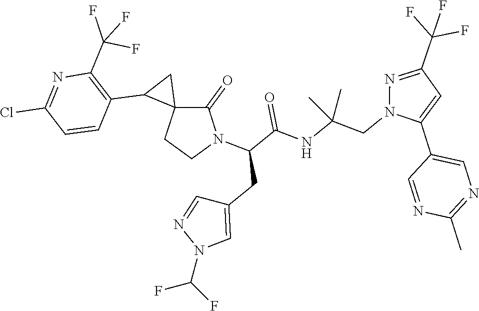 [SMILES: Cc1ncc(-c2cc(C(F)(F)F)nn2CC(C)(C)NC(=O)[C@@H](Cc2cnn(C(F)F)c2)N2CCC3(CC3c3ccc(Cl)nc3C(F)(F)F)C2=O)cn1] | 760 (M + H) |
| 320^{b} | (R)-2-((1S,3R)-1-(4-bromo- 2-chlorophenyl)-4-oxo-5- azaspiro[2.4]heptan-5-yl)-N- (2-methyl-1-(5-(2- methylpyrimidin-5-yl)-3- (trifluoromethyl)-1H- pyrazol-1-yl)propan-2-yl)-3- (1-(methyl-d3)-1H-pyrazol- 4-yl)propanamide | 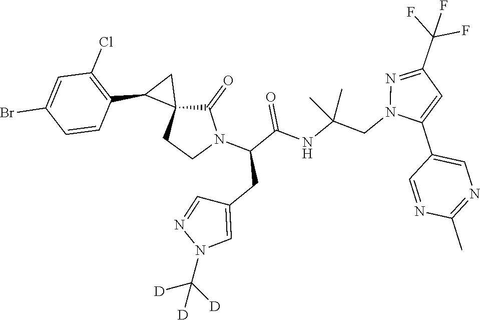 [SMILES: [2H]C([2H])([2H])n1cc(C[C@H](C(=O)NC(C)(C)Cn2nc(C(F)(F)F)cc2-c2cnc(C)nc2)N2CC[C@@]3(C[C@@H]3c3ccc(Br)cc3Cl)C2=O)cn1] | 738 (M + H) |
| 321ª | (2R)-2-(trans)-(1-(6-Chloro- 2-(trifluoromethyl)pyridin-3- yl)-4-oxo-5- azaspiro[2.4]heptan-5-yl)-3- (1-(difluoromethyl)-1H- pyrazol-4-yl)-N-(2-methyl-1- (5-(1-methyl-6-oxo-1,6- dihydropyridin-3-yl)-3- (trifluoromethyl)-1H- pyrazol-1-yl)propan-2- yl)propanamide, isomer 1 | 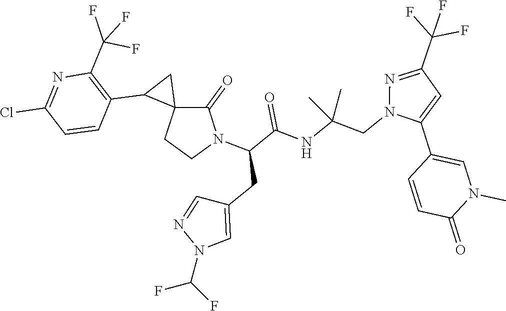 [SMILES: Cn1cc(-c2cc(C(F)(F)F)nn2CC(C)(C)NC(=O)[C@@H](Cc2cnn(C(F)F)c2)N2CCC3(CC3c3ccc(Cl)nc3C(F)(F)F)C2=O)ccc1=O] | 775 (M + H) |
| 322ª | (2R)-2-(trans)-(1-(6-Chloro- 2-(trifluoromethyl)pyridin-3- yl)-4-oxo-5- azaspiro[2.4]heptan-5-yl)-N- (2-methyl-1-(5-(2- methylpyrimidin-5-yl)-3- (trifluoromethyl)-1H- pyrazol-1-yl)propan-2-yl)-3- (1-(methyl-d₃)-1H-pyrazol-4- yl)propanamide, isomer 1 | 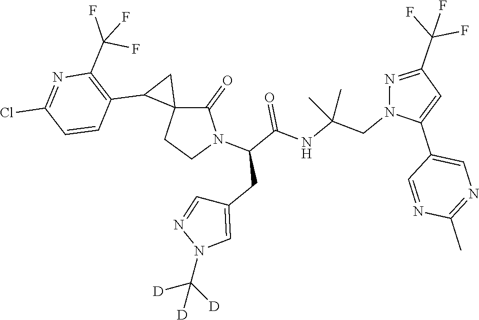 [SMILES: [2H]C([2H])([2H])n1cc(C[C@H](C(=O)NC(C)(C)Cn2nc(C(F)(F)F)cc2-c2cnc(C)nc2)N2CCC3(CC3c3ccc(Cl)nc3C(F)(F)F)C2=O)cn1] | 727 (M + H) |
| 323^{c} | (R)-2-((trans)-1-(4-Bromo-2- (difluoromethyl)phenyl)-4- oxo-5-azaspiro[2.4]heptan-5- yl)-3-(1-(difluoromethyl)- 1H-pyrazol-4-yl)-N-(1-(5-(5- fluoro-1-methyl-6-oxo-1,6- dihydropyridin-3-yl)-3- (trifluoromethyl)-1H- pyrazol-1-yl)-2- methylpropan-2- yl)propanamide | 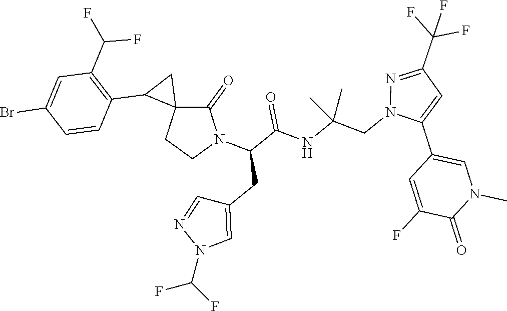 [SMILES: Cn1cc(-c2cc(C(F)(F)F)nn2CC(C)(C)NC(=O)[C@@H](Cc2cnn(C(F)F)c2)N2CCC3(CC3c3ccc(Br)cc3C(F)F)C2=O)cc(F)c1=O] | 818, 820 (M + H) |
| 324^{d} | (R)-2-((1R,3R)-1-(4- Bromophenyl)-4-oxo-5- azaspiro[2.4]heptan-5-yl)-N- (1-(5-(2- cyclopropylpyrimidin-5-yl)- 3-(trifluoromethyl)-1H- pyrazol-1-yl)-2- methylpropan-2-yl)-3-(1- (difluoromethyl)-1H-pyrazol- 4-yl)propanamide | 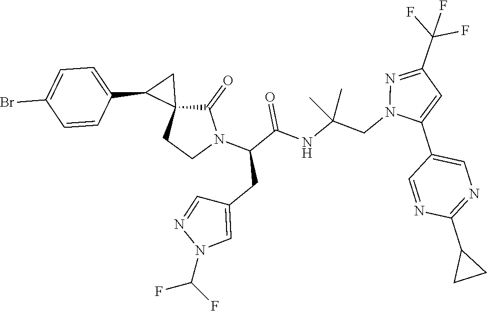 [SMILES: CC(C)(Cn1nc(C(F)(F)F)cc1-c1cnc(C2CC2)nc1)NC(=O)[C@@H](Cc1cnn(C(F)F)c1)N1CC[C@@]2(C[C@@H]2c2ccc(Br)cc2)C1=O] | 761, 763 (M + H) |
| 325^{d} | (R)-2-((1R,3R)-1-(4- Bromophenyl)-4-oxo-5- azaspiro[2.4]heptan-5-yl)-N- (1-(5-(2- trifluoromethylpyrimidin-5- yl)-3-(trifluoromethyl)-1H- pyrazol-1-yl)-2- methylpropan-2-yl)-3-(1- (difluoromethyl)-1H-pyrazol- 4-yl)propanamide | 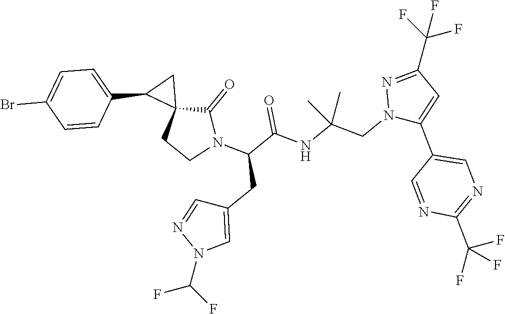 [SMILES: CC(C)(Cn1nc(C(F)(F)F)cc1-c1cnc(C(F)(F)F)nc1)NC(=O)[C@@H](Cc1cnn(C(F)F)c1)N1CC[C@@]2(C[C@@H]2c2ccc(Br)cc2)C1=O] | 789, 792 (M + H) |
| 326 | (2R)-2-((trans)-1-(6-chloro- 2-methylpyridin-3-yl)-4-oxo- 5-azaspiro[2.4]heptan-5-yl)- 3-(1-(difluoromethyl)-1H- pyrazol-4-yl)-N-(2-methyl-1- (5-(2-methylpyrimidin-5-yl)- 3-(trifluoromethyl)-1H- pyrazol-1-yl)propan-2- yl)propanamide | 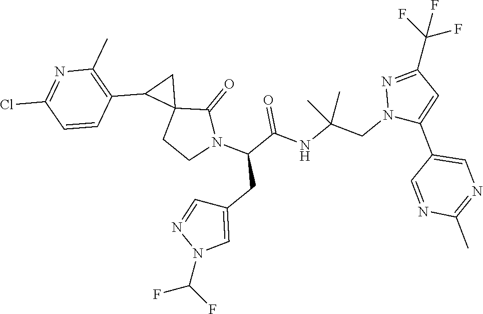 [SMILES: Cc1ncc(-c2cc(C(F)(F)F)nn2CC(C)(C)NC(=O)[C@@H](Cc2cnn(C(F)F)c2)N2CCC3(CC3c3ccc(Cl)nc3C)C2=O)cn1] | 706 (M + H) |
| 327^{e} | (R)-2-((1R,3R)-1-(4- bromophenyl)-4-oxo-5- azaspiro[2.4]heptan-5-yl)-3- (1-(difluoromethyl)-1H- pyrazol-4-yl)-N-(2-methyl-1- (5-(1-methyl-6-oxo-1,6- dihydropyridin-3-yl)-3- (trifluoromethyl)-1H- pyrazol-1-yl)propan-2- yl)propanamide | 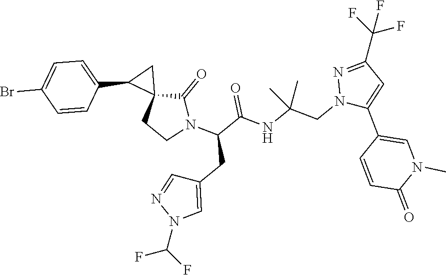 [SMILES: Cn1cc(-c2cc(C(F)(F)F)nn2CC(C)(C)NC(=O)[C@@H](Cc2cnn(C(F)F)c2)N2CC[C@@]3(C[C@@H]3c3ccc(Br)cc3)C2=O)ccc1=O] | 750, 752 (M + H) |

^{a}The reaction was quenched with FA (10 eq) as a solution in H₂O, and the compound was purified with ACN/0.1% FA in water;
^{b}The reaction was quenched with FA (7 eq) as a solution in H₂O, and the compound was purified with ACN/0.1% FA in water;
^{c}Prepared as described with 0.1 equiv TBAI. Purified by reverse phase flash chromatography using a gradient of 0-100% ACN/10 mM aq. NH₄HCO₃ (5% MeOH);
^{d}The reaction mixture was partitioned between 20 mL water and 20 mL EtOAc and extracted with EtOAc (2 × 30 mL), dried over Na₂SO₄, filtered, and concentrated under reduced pressure to provide titled compound without purification;
^{e}The reaction mixture was partitioned between sat. aq. sodium bicarbonate (20 mL), H2O (15 mL) and EtOAc:Toluene (1:1)(30 mL) and organic layers was separated. The aqueous layer was extracted with EtOAc:Toluene (1:1)(3 × 40 mL). The combined organic layers were washed with brine, dried over Na₂SO₄, filtered, and concentrated under reduced pressure to provide titled compound without further purification.

### Preparation 328

### (2R)-3-(1-(Difluoromethyl)-1H-pyrazol-4-yl)-2-(1S,3R)-(1-(4-(5,5-dimethyl-1,3,2-dioxaborinan-2-yl)-2-fluorophenyl)-4-oxo-5-azaspiro[2.4]heptan-5-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide

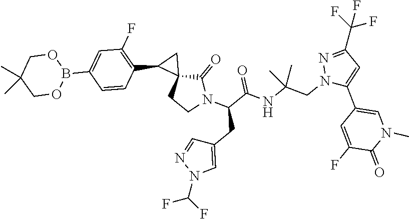

A stirring mixture of (2R)-2-(1S,3R)-(1-(4-bromo-2-fluorophenyl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (2.42 g, 3.08 mmol), bis(neopentyl glycolato) diborane (2.08 g, 9.23 mmol), KOAc (1.21 g, 12.31 mmol) in 1,4-dioxane (31 mL) was sparged with nitrogen for 5 min. Then Pd(dppf)Cl₂·CH₂Cl₂ (377 mg, 462 μmol) was added and sparged with nitrogen for additional 5 min. Then reaction vessel was sealed and heated to 80° C. for 3 h. Then the reaction mixture was cooled down to RT and used directly in the next step. ES-MS m/z 752 (corresponding boronic acid M+H).

The compounds in the following table were prepared as described in Preparation 328. Bis(pinacolato)diboron is a suitable replacement of Bis(neopentyl glycolato) diboran. Reaction times (1 h to overnight) and temperatures (80° C. to 100° C.) may vary, and differing methods can be used to work up or purify the compounds (normal phase, or high, neutral or low pH reversed phase). Such variances would be apparent to one skilled in the art.

TABLE 24

| Prepa- | | | |
|---|---|---|---|
| ra- | | | ES-MS |
| tion | Chemical Name | Structure | m/z |
| 329 | (2R)-3-(1-(Difluoromethyl)- 1H-pyrazol-4-yl)-2-(trans)-(1- (2-(difluoromethyl)-4-(4,4,5,5- tetramethyl-1,3,2- dioxaborolan-2-yl)phenyl)-4- oxo-5-azaspiro[2.4]heptan-5- yl)-N-(1-(5-(5-fluoro-1- methyl-6-oxo-1,6- dihydropyridin-3-yl)-3- (trifluoromethyl)-1H-pyrazol- 1-yl)-2-methylpropan-2- yl)propanamide | 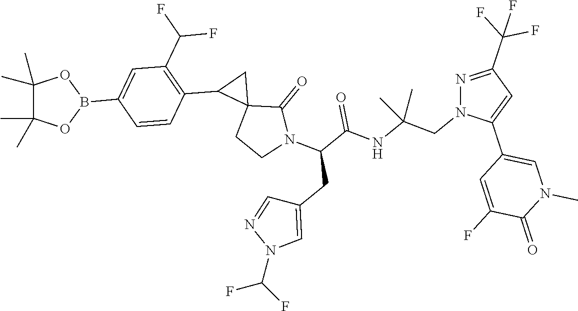 | 866 (M + H) |
| 330 | (R)-3-(1-(Difluoromethyl)-1H- pyrazol-4-yl)-N-(2-methyl-1- (3-(trifluoromethyl)-5-(2- (trifluoromethyl)pyrimidin-5- yl)-1H-pyrazol-1-yl)propan-2- yl)-2-((1R,3R)-4-oxo-1-(4- (4,4,5,5-tetramethyl-1,3,2- dioxaborolan-2-yl)phenyl)-5- azaspiro[2.4]heptan-5- yl)propanamide | 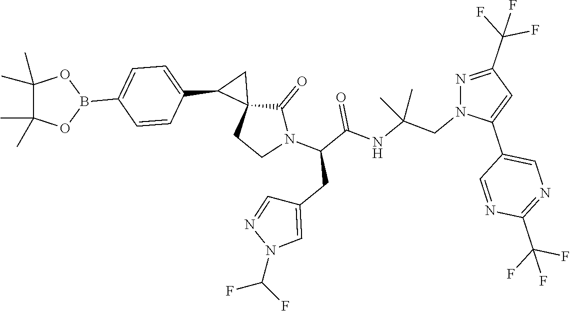 [SMILES: CC(C)(Cn1nc(C(F)(F)F)cc1-c1cnc(C(F)(F)F)nc1)NC(=O)[C@@H](Cc1cnn(C(F)F)c1)N1CC[C@@]2(C[C@@H]2c2ccc(B3OC(C)(C)C(C)(C)O3)cc2)C1=O] | 807 (M − H) |
| 331 | (R)-N-(1-(5-(2- Cyclopropylpyrimidin-5-yl)-3- (trifluoromethyl)-1H-pyrazol- 1-yl)-2-methylpropan-2-yl)-3- (1-(difluoromethyl)-1H- pyrazol-4-yl)-2-((1R,3R)-4- oxo-1-(4-(4,4,5,5-tetramethyl- 1,3,2-dioxaborolan-2- yl)phenyl)-5- azaspiro[2.4]heptan-5- yl)propanamide | 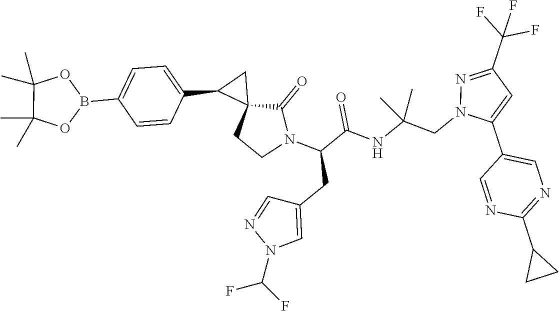 [SMILES: CC(C)(Cn1nc(C(F)(F)F)cc1-c1cnc(C2CC2)nc1)NC(=O)[C@@H](Cc1cnn(C(F)F)c1)N1CC[C@@]2(C[C@@H]2c2ccc(B3OC(C)(C)C(C)(C)O3)cc2)C1=O] | 835 (M − H) |
| 332 | (R)-3-(1-(difluoromethyl)-1H- pyrazol-4-yl)-N-(2-methyl-1- (5-(1-methyl-6-oxo-1,6- dihydropyridin-3-yl)-3- (trifluoromethyl)-1H-pyrazol- 1-yl)propan-2-yl)-2-((1R,3R)- 4-oxo-1-(4-(4,4,5,5- tetramethyl-1,3,2- dioxaborolan-2-yl)phenyl)-5- azaspiro[2.4]heptan-5- yl)propanamide | 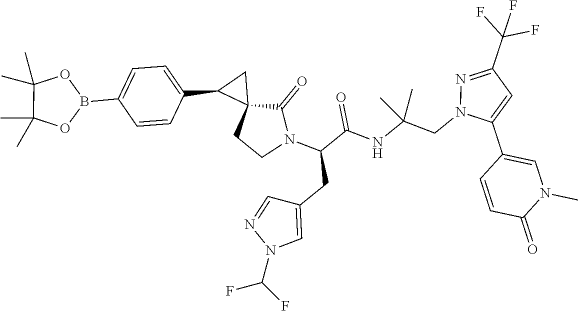 | 798 (M − H) |

### Preparation 333

### (1R,2S)-1-(2-Chloroethyl)-N—((R)-3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-2-(2-fluoro-4-(2-methyl-2H-pyrazolo[3,4-b]pyridin-5-yl)phenyl)cyclopropane-1-carboxamide

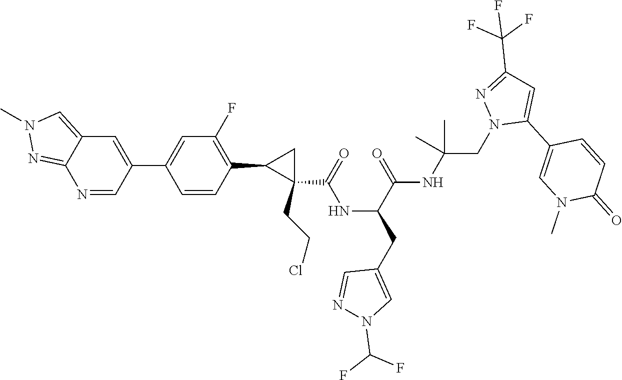 [SMILES: Cn1cc2cc(-c3ccc([C@H]4C[C@]4(CCCl)C(=O)N[C@H](Cc4cnn(C(F)F)c4)C(=O)NC(C)(C)Cn4nc(C(F)(F)F)cc4-c4ccc(=O)n(C)c4)c(F)c3)cnc2n1]

A mixture of (1S,3R)-1-(2-fluoro-4-(2-methyl-2H-pyrazolo[3,4-b]pyridin-5-yl)phenyl)-5-oxaspiro[2.4]heptan-4-one (300 mg, 889 μmol) and benzyltriethylammonium chloride (408 mg, 1.78 mmol) in DCE (4.45 mL) was treated with SOCl₂ (260 μL, 3.56 mmol) followed by BF₃·Et₂O (220 μL, 1.78 mmol). After stirring at 80° C. overnight, the reaction mixture was cooled to RT and concentrated under reduced pressure. DCE (5 mL) was added, and the mixture was concentrated under reduced pressure to obtain a quantitative yield of (1R,2R)-1-(2-chloroethyl)-2-(2-fluoro-4-(2-methyl-2H-pyrazolo[3,4-b]pyridin-5-yl)phenyl)cyclopropane-1-carbonyl chloride as a dark sticky oil. In a separate vessel, a solution of (R)-2-amino-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide dihydrochloride (512 mg, 891 μmol) in DCM (10 mL) was stirred at 0° C. and treated with TEA (1.12 mL, 8.04 mmol). A suspension of (1R,2R)-1-(2-chloroethyl)-2-(2-fluoro-4-(2-methyl-2H-pyrazolo[3,4-b]pyridin-5-yl)phenyl)cyclopropane-1-carbonyl chloride (889 μmol) in DCM (10 mL) was added dropwise. The cooling bath was removed, and the reaction mixture was stirred at RT for 4 h. EtOAc (200 mL) and cold water (200 mL) were added, and the layers were separated. The aq. layer was extracted with EtOAc (100 mL), and the organic layers were combined, washed with water (3 x) and sat. aq. NaCl, dried over Na₂SO₄, filtered, and concentrated under reduced pressure to obtain a quantitative yield of the title compound as a light-brown solid. ES-MS m/z 857 (M+H).

The compounds in the following table were prepared as described in Preparation 333 using the appropriate lactone and appropriate amine or amine salt. DIEA is a suitable replacement for TEA. Reaction time may vary. Slight variations to the reaction conditions and purification methods would be apparent to one skilled in the art.

TABLE 25

| Prepara- | | | ES-MS |
|---|---|---|---|
| tion | Chemical Name | Structure | m/z |
| 334ª | (1R,2S)-1-(2-Chloroethyl)- N-((R)-3-(1- (difluoromethyl)-1H-pyrazol- 4-yl)-1-((1-(5-(5-fluoro-1- methyl-6-oxo-1,6- dihydropyridin-3-yl)-3- (trifluoromethyl)-1H- pyrazol-1-yl)-2- methylpropan-2-yl)amino)-1- thioxopropan-2-yl)-2-(2- fluoro-4-(2-methyl-2H- pyrazolo[3,4-b]pyridin-5- yl)phenyl)cyclopropane-1- carboxamide, isomer 1 | 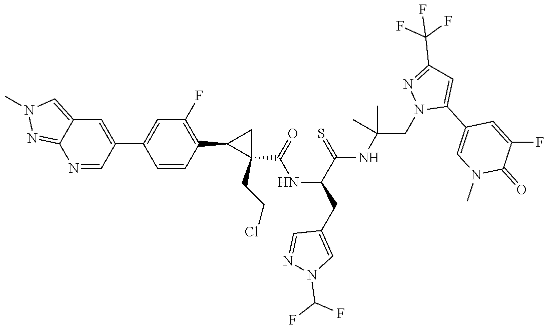 [SMILES: Cn1cc2cc(-c3ccc([C@H]4C[C@]4(CCCl)C(=O)N[C@H](Cc4cnn(C(F)F)c4)C(=S)NC(C)(C)Cn4nc(C(F)(F)F)cc4-c4cc(F)c(=O)n(C)c4)c(F)c3)cnc2n1] | 891 (M + H) |
| 335^{b} | (1R,2S)-1-(2-Chloroethyl)-N- ((R)-3-(1-(difluoromethyl)- 1H-pyrazol-4-yl)-1-((1-((5- (5-fluoro-1-methyl-6-oxo- 1,6-dihydropyridin-3-yl)-3- (trifluoromethyl)-1H- pyrazol-1- yl)methyl)cyclopropyl)amino)- 1-oxopropan-2-yl)-2-(2- fluoro-4-(2-methyl-2H- pyrazolo[3,4-b]pyridin-5- yl)phenyl)cyclopropane-1- carboxamide, isomer 1 | 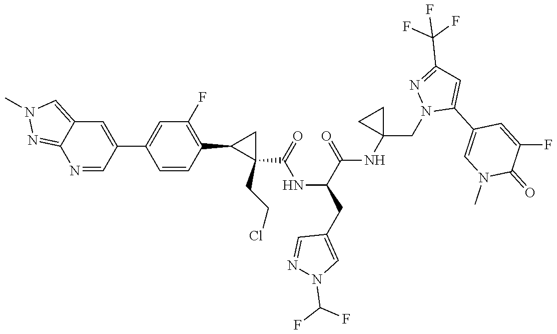 [SMILES: Cn1cc2cc(-c3ccc([C@H]4C[C@]4(CCCl)C(=O)N[C@H](Cc4cnn(C(F)F)c4)C(=O)NC4(Cn5nc(C(F)(F)F)cc5-c5cc(F)c(=O)n(C)c5)CC4)c(F)c3)cnc2n1] | 873 (M + H) |

^{a}1 h reaction time.
^{b}0.5 h reaction time.

### Preparation 336

### (S)-1-(Naphthalen-1-yl) ethan-1-amine (1R,2S)-2-(4-bromo-2-fluorophenyl)-1-(2-hydroxyethyl)cyclopropane-1-carboxylate

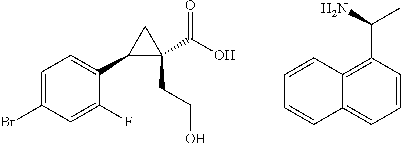 [SMILES: O=C(O)[C@@]1(CCO)C[C@@H]1c1ccc(Br)cc1F ; C[C@H](N)c1cccc2ccccc12]

To a mixture of rac-(trans)-1-(4-bromo-2-fluorophenyl)-5-oxaspiro[2.4]heptan-4-one (197.00 g, 688.89 mmol) in THF (600 mL) and water (300 mL) was added LiOH (87.0 g, 2.07 mol) and the mixture was stirred for 2 h. The mixture was concentrated under reduced pressure at 40° C., then 1 N HCl was added to adjust the pH to 3. The resulting suspension was filtered, and the filter cake was washed with water and ACN, then dried under reduced pressure to give rac-(trans)-2-(4-bromo-2-fluorophenyl)-1-(2-hydroxyethyl)cyclopropane-1-carboxylic acid (180 g) as a solid.

A mixture of rac-(trans)-2-(4-bromo-2-fluorophenyl)-1-(2-hydroxyethyl)cyclopropane-1-carboxylic acid (176.0 g, 580 mmol) in EtOAc (1.6 L) was stirred at 35° C. for 30 min. (S)-1-(1-Naphthyl)ethylamine (65.0 g, 379.6 mmol) was added, and the resulting mixture was stirred for 16 h at 25° C. (internal temperature). The resulting precipitate was filtered, then triturated by stirring with MTBE (1 L) at 25° C. for 16 h. Filtration gave the title compound (105 g, 38%) as a white solid.

A sample of the title compound was dissolved in EtOH (1 mL) and used for analytical SFC ˜97.8% ee, Rt=2.87 min, isomer 2 (Chiralpak® AD-3, 4.6×150 mm, 3 μm, column temperature 35° C., 220 nm, flow rate 2.5 mL/min, isocratic 20% EtOH (with 0.5% IPAm): 80% CO₂; Waters® ACQUITY UPC²® with PDA).

### Preparation 337

### (1S,3R)-1-(4-Bromo-2-fluorophenyl)-5-oxaspiro[2.4]heptan-4-one

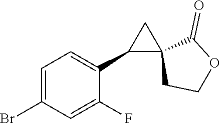 [SMILES: O=C1OCC[C@@]12C[C@@H]2c1ccc(Br)cc1F]

A mixture of(S)-1-(naphthalen-1-yl) ethan-1-amine (1R,2S)-2-(4-bromo-2-fluorophenyl)-1-(2-hydroxyethyl)cyclopropane-1-carboxylate (100.0 g, 210 mmol) in ACN (1 L) was treated with 12 M HCl (200 mL) dropwise, and the resulting mixture was stirred at 25° C. for 2 h. Water (100 mL) was added while stirring. The resulting suspension was filtered to obtain the white solid. Treated the white solid with 1M HCl 200 ml and stirred for 2 hr at 25° C., filtered and dried under reduced pressure to give the title compound (53 g, 88%) as a white solid. ES-MS m z 285,287 (M+H). Analytical SFC ˜99.9% ee, Rt=1.28 min, isomer 1 (Chiralpak® IG-3, 4.6× 100 mm, 3 μm, column temperature 40° C., 220 nm, flow rate 4.0 mL/min; 15% MeOH (with 0.2% isopropyl amine): 85% CO₂; Waters® ACQUITY UPC²® with PDA).

### Preparation 338

### 5-Fluoro-1-methyl-1H-pyrazolo[3,4-b]pyridine

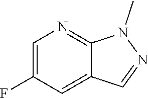

To a mixture of 5-fluoro-1H-pyrazolo[3,4-b]pyridine (1.8 g, 13.13 mmol) in DMSO (100 mL) was added NaHMDS (1.0 M in THF, 19.7 mL, 19.7 mmol) and iodomethane (2.46 mL, 39.3 mmol). The reaction was allowed to stir at RT for 2 h, then quenched with water and extracted three times with EtOAc. The organic layers were combined, washed three times with water, dried over Na₂SO₄, filtered and concentrated under reduced pressure. The residue was purified on silica gel, eluting with 0-40% EtOAc in cyclohexane to give the title compound as a white solid (1.3 g, 66%). ¹H-NMR (400 MHz, DMSO-d₆) δ 8.64-8.58 (m, 1H), 8.19-8.11 (m, 2H), 4.07 (s, 3H).

### Preparation 339

### 5-Bromo-6-fluoro-1-methyl-1H-pyrazolo[4,3-b]pyridine

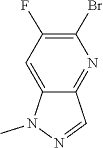

A mixture of 5-bromo-6-fluoro-1H-pyrazolo[4,3-b]pyridine (1.95 g, 80 wt %, 7.22 mmol) in DMF (20 mL) was purged with N₂ three times, then 60% NaH in mineral oil (347 mg, 60 wt %, 8.67 mmol) was added to the mixture at 0° C. The mixture was stirred at 0° C. for 30 min under N₂, then iodomethane (1.55 g, 708 μL, 10.8 mmol) was added. The mixture was stirred at 0° C. for 1 h under N₂, then it was poured into H₂O (80 mL) and extracted with EtOAc (60 mL×3). The combined organic layers were washed with sat. aq. NaCl (60 mL×3), dried over anhydrous Na₂SO₄, filtered, and concentrated under reduced pressure. The residue was purified on silica gel, eluting with 6-7% EtOAc in 4:1 hexanes: DCM to obtain the title compound (746 mg, 44% yield) as a yellow solid. ES-MS m/z 230, 232 (M+H).

### Preparation 340

### 6-Bromo-1-methyl-1H-pyrazolo[4,3-b]pyridine

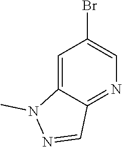

The following procedure was performed in 14 reactions carried out in parallel on the scale given herein. A mixture of 6-bromo-1H-pyrazolo[4,3-b]pyridine (500 g, 2.52 mol) and K₂CO₃ (523 g, 3.79 mol) in DMF (3.50 L) was degassed and purged with N₂ 3 times. To the mixture was slowly added iodomethane (430 g, 3.03 mol) at 25-35° C. The mixture was stirred at 25° C. for 6 h under N₂ atmosphere. Every two reactions were combined for workup. The reaction mixtures were quenched by H₂O (21.0 L) at 0-5° C. and extracted with EtOAc (1.00 L×6). The combined organic layers were washed with sat. aq. NaCl (6.00 L×2), then the organic layers concentrated under reduced pressure.

All fourteen reactions were combined for purification, combining the residues from the aqueous workups. The combined product was purified on silica gel, eluting with 5-11% EtOAc in PE and then triturated with MTBE (3.50 L) at 25° C. for 1 h. The solid was collected by filtration and dried under reduced pressure to give the title compound (3.51 kg, 47%) as a yellow solid. ES-MS m/z 212 (M+H).

### Preparation 341

### 1-Methyl-1H-pyrazolo[4,3-b]pyridine-6-carbonitrile

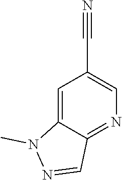

The following procedure was performed in 4 reactions carried out in parallel on the scale given herein. A mixture of 6-bromo-1-methyl-1H-pyrazolo[4,3-b]pyridine (104 g, 490 mmol), Pd(PPh₃)₄ (113 g, 98.0 mmol), and Zn(CN)₂ (85.5 g, 728 mmol) in DMF (1.04 L) was degassed and purged with N₂ 3 times. The mixture was stirred at 120° C. for 12 h under N₂ atmosphere. The four reaction mixtures were combined, filtered, and water (12.0 L) was added to the filtrate, and the mixture was extracted with EtOAc (3.20 L×5). The combined organic layers were washed with sat. aq. NaCl (1.00 L) and the organic layers were concentrated under reduced pressure. The residue was purified on silica gel, eluting with 0-100% EtOAc in PE to afford the title compound (300 g, 89% yield) as a white solid. ES-MS m/z 159 (M+H).

### Preparation 342

### 5-Fluoro-1-methyl-1H-pyrazolo[3,4-b]pyridine 7-oxide

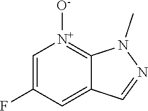

To 5-fluoro-1-methyl-1H-pyrazolo[3,4-b]pyridine (800 mg, 5.29 mmol) and EtOAc (50 mL) was added 3-chloroperoxybenzoic acid (8.7 mL, 75% Wt, 21.2 mmol). Reaction was heated to 45° C. overnight. Sat. aq. NaHCO₃ was added, and the aqueous layer was extracted with EtOAc (3×50 mL). The residue was purified by silica gel chromatography, eluting with 0-40% 3:1 EtOH/EtOAc in cyclohexane to obtain the title compound (666 mg, 75%) as a white solid. ES-MS m/z 168 (M+H).

### Preparation 343

### 6-Cyano-1-methyl-1H-pyrazolo[4,3-b]pyridine 4-oxide

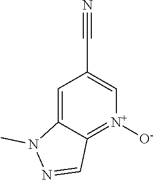

The following procedure was performed in 3 reactions carried out in parallel on the scale given herein. A mixture of 1-methyl-1H-pyrazolo[4,3-b]pyridine-6-carbonitrile (100 g, 632 mmol), methyltrioxorhenium (15.8 g, 63.2 mmol), and 30% aq. H₂O₂ (3.19 mol, 306 mL) in DCM (1.00 L) was degassed and purged with N₂ 3 times. The mixture was stirred at 45° C. for 48 h under N₂ atmosphere. The three reactions were combined for workup. MnO₂ (10 wt %, 30 g) and H₂O (900 mL) were added into the combined reaction mixtures at 45° C. under N₂ and the resulting mixture was stirred for 30 min at 40-45° C. The mixture was filtered, and the filter cake was washed with DCM (600 mL) to provide a mixture of the title compound and MnO₂ (300 g) as a gray solid. TLC (EtOAc) R_{f}=0.3. The mixture was used without further purification.

### Preparation 344

### 5-Chloro-1-methyl-1H-pyrazolo[4,3-b]pyridine-6-carbonitrile

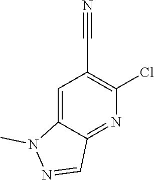

Three reactions were carried out in parallel on the scale given herein. To a solution of 6-cyano-1-methyl-1H-pyrazolo[4,3-b]pyridine 4-oxide (100 g, 574 mmol) and DIEA (111 g, 861 mmol, 150 mL) in DCM (1.00 L) was added oxalyl chloride (87.4 g, 689 mmol) dropwise at 0° C. over 1 h. The resulting mixture was stirred at 0-20° C. for 6 h. The three reactions were combined for workup. The combined reaction mixtures were quenched by water (900 mL) and extracted with DCM (600 mL×2). The combined organic layers were washed with sat. aq. NaCl (900 mL) and the organic phase was concentrated under reduced pressure. The residue was purified on silica gel eluting with 25%-100% EtOAc in PE to obtain the title compound (200 g, 60% yield) as an off-white solid. ES-MS m/z 193 (M+H).

### Preparation 345

### 6-Chloro-5-fluoro-1-methyl-1H-pyrazolo[3,4-b]pyridine

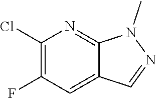

5-Fluoro-1-methyl-1H-pyrazolo[3,4-b]pyridine 7-oxide (500 mg, 2.99 mmol) was dissolved in DCM (20 mL) under N₂. The flask was cooled to 0° C., then TEA (1.04 mL, 7.46 mmol) and oxalyl chloride (657 μL, 7.48 mmol) were added sequentially. The mixture was allowed to warm to RT over 2 h. The reaction was quenched with H₂O and the aqueous phase was extracted with EtOAc (3×). The combined organic phases were washed with water (3×), dried over Na₂SO₄, and concentrated under reduced pressure. The residue was purified on silica gel eluting with 0-20% 4:1 MeOH/DCM in DCM to obtain the title compound (170 mg, 31% yield) as a white solid. ¹H-NMR (400 MHz, CDCl₃) δ 7.99 (s, 1H), 7.80 (d, J=7.6 Hz, 1H), 4.15 (s, 3H).

### Preparation 346

### 3,5-Dichloro-1-methyl-1H-pyrazolo[4,3-b]pyridine-6-carbonitrile

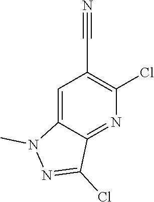

A mixture of 5-chloro-1-methyl-1H-pyrazolo[4,3-b]pyridine-6-carbonitrile (1.0 g, 5.2 mmol), NCS (0.83 g, 6.2 mmol), and DMF (10 mL) under nitrogen atmosphere was heated to 50° C. for 25 h. Additional NCS (350 mg, 2.6 mmol) was added and the mixture was heated to 50° C. for 23 h. The mixture was then diluted with water (20 mL) and heated to reflux for 1 min, then cooled to RT and filtered. The resulting solid was dissolved in MeOH (40 mL) at reflux then cooled to RT. Vacuum filtration gave the title compound (800 mg, 68% yield) as a white solid. ES-MS m/z 227 (M+H).

### Preparation 347

### (2-Methylimidazo[1,2-a]pyrimidin-6-yl) boronic acid

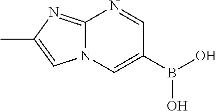

6-Bromo-2-methylimidazo[1,2-a]pyrimidine (800 g, 3.77 moles) was dissolved in toluene (5.6 L) at RT under N₂ protection. Pd(PPh₃)₂Cl₂ (79.5 g, 0.03 eq.) and KOAc (1111 g, 3.0 eq.) were added in one portion, followed by bis(pinacolato)diboron (1054 g, 1.1 eq.). The reaction was stirred at 90-100° C. for 16 h. The reaction was cooled to 25° C., then DCM (5.6 L) was added and the mixture was stirred for 0.5 h. The mixture was filtered, eluting with DCM (1 L×2), and the filtrate was concentrated under reduced pressure to obtain a brown residue. PE (5.6 L) was added and the mixture was stirred for 0.5 h. The mixture was then filtered and filter cake was rinsed with PE (500 mL×2). The solid was combined with the crude material from other reactions (2850 g) and the combined solid was slurried in EtOH/H₂O (5/1, 3 L) for 1 h. The mixture was filtered and the filter cake was rinsed with EtOH (300 mL×2). The filter cake was then slurried in ACN (3 L) for 1 h. The mixture was filtered and the cake was rinsed with ACN (300 mL×2). The solid was dried by air-blowing in the oven for 16 h at 50-60° C. to obtain the title compound (1560 g, 70%) as an off-white solid. ES-MS m/z 178.1 (M+H).

### Preparation 348

### (2-Cyclopropylimidazo[1,2-a]pyrimidin-6-yl) boronic acid

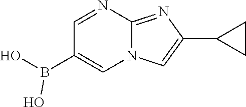 [SMILES: OB(O)c1cnc2nc(C3CC3)cn2c1]

6-Bromo-2-cyclopropylimidazo[1,2-a]pyrimidine (1.20 g, 5.03 mmol) was charged with bis(pinacolato)diboron (3.81 g, 15 mmol), KOAc (1.51 g, 15.39 mmol), Pd(ddpf)Cl₂ (DCM adduct, 416.1 mg, 510 μmol) and 1,4-dioxane (20 mL). The mixture was purged with N₂ for 3 mins and then heated to 80° C. under N₂. The reaction mixture was cooled to RT to obtain a quantitative yield of the title compound as a solution, which was carried forward without further purification. ES-MS m/z 204 (M+H).

### Preparation 349

### 1-Methyl-5-(tributylstannyl)pyrimidin-2(1H)-one

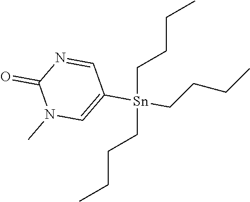 [SMILES: CCC[CH2][Sn]([CH2]CCC)([CH2]CCC)[c]1cnc(=O)n(C)c1]

To a mixture of 5-bromo-1-methylpyrimidin-2(1H)-one (300 mg, 1.57 mmol) in 1,4-dioxane (5 mL) were added 1,1,1,2,2,2-hexabutyldistannane (1.4 mL, 2.8 mmol) and bis(di-tert-butyl (4-dimethylaminophenyl)phosphine)dichloropalladium (II) (115 mg, 162 μmol). The reaction mixture was degassed and purged with N₂ 3 times, then stirred at 105° C. for 16 h. The reaction mixture was quenched with sat. aq. KF (30 mL) and extracted with EtOAc (30 mL×3). The combined organic layers were dried over anhydrous Na₂SO₄, filtered, and concentrated under reduced pressure. The residue was purified by silica gel chromatography using a gradient of 0-5% MeOH in DCM to give the title compound (264 mg, 38%) as a yellow oil. ES-MS m z 401 (M+H).

### Preparation 350

### tert-Butyl (2-methyl-1-(4-(trifluoromethyl)-1H-imidazol-2-yl)propan-2-yl)carbamate

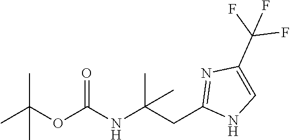 [SMILES: CC(C)(Cc1nc(C(F)(F)F)c[nH]1)NC(=O)OC(C)(C)C]

To a mixture of tert-butyl-(2-methyl-4-oxobutan-2-yl)carbamate (10.0 g, 49.7 mmol) in EtOH (150 mL) was added ammonium hydroxide (54.9 mL, 397 mmol). The reaction was cooled to 0° C. and 1,1,1-trifluoro-3,3-dibromoacetone (102 mL, 745 mmol) was added. The reaction was stirred at RT for 16 h, then was poured into water (300 mL) and extracted with EtOAc (300 mL×2). The combined organic layers were dried over anhydrous Na₂SO₄, then filtered and concentrated under reduced pressure. The residue was purified by silica gel column chromatography (gradient 0-5% MeOH in DCM) to obtain the title compound (5.4 g, 96% purity, 34%) as a white solid. ES-MS m/z 308 (M+H).

### Preparation 351

### tert-Butyl (2-methyl-1-(1-(1-methyl-2-oxopyrrolidin-3-yl)-4-(trifluoromethyl)-1H-imidazo

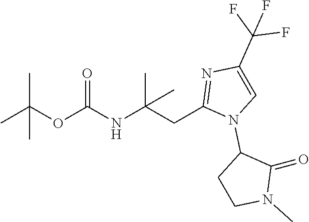 [SMILES: CN1CCC(n2cc(C(F)(F)F)nc2CC(C)(C)NC(=O)OC(C)(C)C)C1=O]

To tert-butyl (2-methyl-1-(4-(trifluoromethyl)-1H-imidazol-2-yl)propan-2-yl)carbamate (897.2 mg, 2.92 mmol) was added K₂CO₃ (1.26 g, 9.13 mmol). The vial was purged with N₂ for 3 min. DMF (8.0 mL) was added followed by 3-bromo-1-methyl-pyrrolidin-2-one (650 μL, 5.87 mmol) and the vial was heated to 60° C. under N₂ overnight. The reaction mixture was cooled to RT and then partitioned between 55 mL EtOAc and 55 mL H₂O. The aqueous layer was extracted with EtOAc (25 mL×3). The combined organic layers were washed with sat. aq. NaCl, (25 mL×3) dried over anhydrous Na₂SO₄, filtered and concentrated under reduced pressure. The residue was purified by silica gel chromatography (gradient 0-100% EtOAc in hexanes) to provide the title compound (626.7 mg, 42%) as a beige solid. ES-MS m/z 405 (M+H).

### Preparation 352

### 3-(2-(2-Amino-2-methylpropyl)-4-(trifluoromethyl)-1H-imidazol-1-yl)-1-methylpyrrolidin-2-one hydrochloride (racemic mixture)

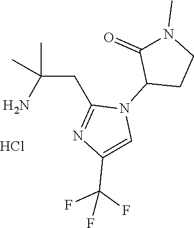 [SMILES: Cl ; CN1CCC(n2cc(C(F)(F)F)nc2CC(C)(C)N)C1=O]

tert-Butyl (2-methyl-1-(1-(1-methyl-2-oxopyrrolidin-3-yl)-4-(trifluoromethyl)-1H-imidazol-2-yl)propan-2-yl)carbamate (626.7 mg, 1.55 mmol) was dissolved in MeOH (3 mL), then 4 M hydrogen chloride in 1,4-dioxane (3 mL, 0.01 mol) was added. The reaction was stirred overnight then was concentrated under reduced pressure to provide the title compound (574.0 mg, 98%) as a yellow solid. ES-MS m/z 305 (M+H).

### Preparation 353

### tert-Butyl (1-(5-(1-cyclopropyl-2-oxo-1,2-dihydropyridin-4-yl)-3-(trifluoromethyl)-1H-pyrazol-1-yl)-2-methylpropan-2-yl)carbamate

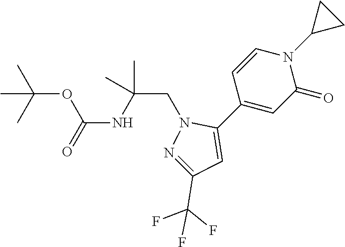 [SMILES: CC(C)(Cn1nc(C(F)(F)F)cc1-c1ccn(C2CC2)c(=O)c1)NC(=O)OC(C)(C)C]

4-Bromo-1-cyclopropylpyridin-2(1H)-one (9.35 g, 39.3 mmol), 1 M aq. Na₂CO₃ (99 mL, 99 mmol) and BrettPhos Pd G3 (3.08 g, 3.36 mmol) in 1,4-dioxane (120 mL) and IPA (30 mL) was degassed and refilled with N₂ 3 times, then stirred at 25° C. for several minutes. tert-Butyl (1-(5-(1,3,6,2-dioxazaborocan-2-yl)-3-(trifluoromethyl)-1H-pyrazol-1-yl)-2-methylpropan-2-yl)carbamate (14.05 g, 33.1 mmol) was added and the mixture was degassed and refilled with N₂ 3 more times, then stirred at 65° C. for 16 h. The reaction mixture was cooled to RT and filtered. The filter cake was washed with EtOAc (50 mL×3), then the filtrate was concentrated under reduced pressure. The residue was diluted with H₂O (30 mL) and extracted with EtOAc (100 mL×2). The combined organic layers were washed with sat. aq. NaCl (100 mL×2), dried over Na₂SO₄, filtered, and concentrated under reduced pressure. The residue was purified by silica gel chromatography (gradient 0-40% EtOAc in hexanes) to provide the title compound (8.15 g, 53%) as a yellow solid. ES-MS m/z 441 (M+H).

### Preparation 354

### tert-Butyl (1-(5-(6-methoxypyridazin-3-yl)-3-(trifluoromethyl)-1H-pyrazol-1-yl)-2-methylpropan-2-yl)carbamate

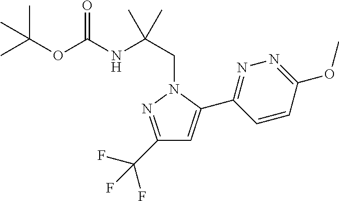 [SMILES: COc1ccc(-c2cc(C(F)(F)F)nn2CC(C)(C)NC(=O)OC(C)(C)C)nn1]

The title compound was prepared as described in Preparation 353 using 3-chloro-6-methoxypyridazine. ES-MS m/z 416 (M+H).

### Preparation 355

### tert-Butyl (2-methyl-1-(5-(2-(tetrahydro-2H-pyran-2-yl)-2H-1,2,3-triazol-4-yl)-3-(trifluoromethyl)-1H-pyrazol-1-yl)propan-2-yl)carbamate

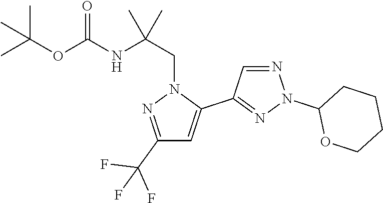 [SMILES: CC(C)(Cn1nc(C(F)(F)F)cc1-c1cnn(C2CCCCO2)n1)NC(=O)OC(C)(C)C]

To a mixture of tert-butyl (1-(5-bromo-3-(trifluoromethyl)-1H-pyrazol-1-yl)-2-methylpropan-2-yl)carbamate (500 mg, 1.29 mmol), 2-(tetrahydro-2H-pyran-2-yl)-4-(4,4,5,5-tetramethyl-1,3,2-dioxaborolan-2-yl)-2H-1,2,3-triazole (505 mg, 1.81 mmol) and Na₂CO₃ (277 mg, 2.61 mmol) in 1,4-dioxane (6 mL) and water (0.6 mL) was added Pd(dppf)Cl₂ (188 mg, 257 μmol). The mixture was degassed and purged with N₂ 3 times. Then the reaction mixture was stirred at 90° C. for 6 h then cooled to RT overnight. The reaction mixture was filtered and the filter cake was washed with EtOAc (10 mL×3). The filtrate was concentrated under reduced pressure then purified on silica gel eluting with 0-17% EtOAc in hexanes to afford the title compound (548 mg, 88%) as a yellow solid. ES-MS m/z 459 (M+H).

### Preparation 356

### 4-(1-(2-Amino-2-methylpropyl)-3-(trifluoromethyl)-1H-pyrazol-5-yl)-1-cyclopropylpyridin-2(1H)-one hydrochloride

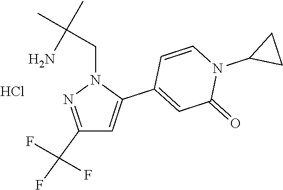 [SMILES: Cl ; CC(C)(N)Cn1nc(C(F)(F)F)cc1-c1ccn(C2CC2)c(=O)c1]

To tert-butyl (1-(5-(1-cyclopropyl-2-oxo-1,2-dihydropyridin-4-yl)-3-(trifluoromethyl)-1H-pyrazol-1-yl)-2-methylpropan-2 yl)carbamate (8.1 g, 17 mmol) was added DCM (20 mL) then 2 M hydrogen chloride in 1,4-dioxane (90 mL, 0.18 mol). The reaction was stirred at 25° C. for 2 h then concentrated under reduced pressure to provide the title compound (8.04 g, 80% purity, 98%) as a white solid. ES-MS m/z 341 (M+H).

### Preparation 357

### 6-(1-(2-Amino-2-methylpropyl)-3-(trifluoromethyl)-1H-pyrazol-5-yl)pyridazin-3 (2H)-one dihydrochloride

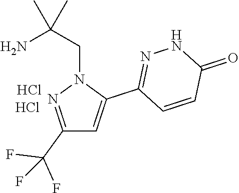 [SMILES: CC(C)(N)Cn1nc(C(F)(F)F)cc1-c1ccc(=O)[nH]n1 ; Cl ; Cl]

The title compound was prepared as described in Preparation 356 using tert-butyl (1-(5-(6-methoxypyridazin-3-yl)-3-(trifluoromethyl)-1H-pyrazol-1-yl)-2-methylpropan-2-yl)carbamate in MeOH, stirring for 12 h at 40° C. ES-MS m/z 302 (M+H).

### Preparation 358

### 2-Methyl-1-(5-(2-(tetrahydro-2H-pyran-2-yl)-2H-1,2,3-triazol-4-yl)-3-(trifluoromethyl)-1H-pyrazol-1-yl)propan-2-amine hydrochloride

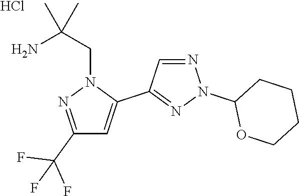 [SMILES: Cl ; CC(C)(N)Cn1nc(C(F)(F)F)cc1-c1cnn(C2CCCCO2)n1]

To a mixture of tert-butyl (2-methyl-1-(5-(2-(tetrahydro-2H-pyran-2-yl)-2H-1,2,3-triazol-4-yl)-3-(trifluoromethyl)-1H-pyrazol-1-yl)propan-2-yl)carbamate (270 mg, 559 μmol) in 1,4-dioxane (2 mL) was added 2.0 M HCl in 1,4-dioxane (3.00 mL, 6.00 mmol). Then the mixture was stirred at 20° C. for 2 h, then 14° C. overnight. The reaction mixture was concentrated under reduced pressure to obtain the title compound as a mixture of tetrahydropyran and non-tetrahydropyran protected analogues (292 mg, 69%). ES-MS 358 and 274 (M+H).

### Preparation 359

### tert-Butyl (R)-(1-((1-(5-(1-cyclopropyl-2-oxo-1,2-dihydropyridin-4-yl)-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate

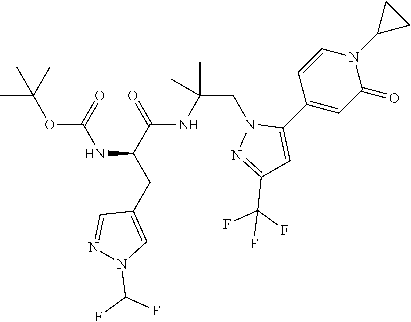 [SMILES: CC(C)(Cn1nc(C(F)(F)F)cc1-c1ccn(C2CC2)c(=O)c1)NC(=O)[C@@H](Cc1cnn(C(F)F)c1)NC(=O)OC(C)(C)C]

To a mixture of 4-(1-(2-amino-2-methylpropyl)-3-(trifluoromethyl)-1H-pyrazol-5-yl)-1-cyclopropylpyridin-2(1H)-one hydrochloride (6.02 g, 80% wt, 12.8 mmol) and (R)-2-((tert-butoxycarbonyl)amino)-3-(1-(difluoromethyl)-1H-pyrazol-4-yl)propanoic acid (3.92 g, 12.8 mmol) was added DMA (60 mL), DIEA (12 mL, 68 mmol) and HATU (5.46 g, 14.1 mmol) at 0° C. The resulting mixture was allowed to warm to 25° C. and stirred for 2 h. The reaction mixture was diluted with H₂O and extracted with EtOAc. The combined organic layers were washed with sat. aq. NaCl, dried over anhydrous Na₂SO₄, filtered, and concentrated under reduced pressure to give a residue (12 g, yellow oil) that was purified by silica gel chromatography (eluting with 0-45% EtOAc in hexanes) to give the title compound (9.76 g, 91%, 75% purity) as a white solid. ES-MS m/z 528 (M-Boc+H).

### Preparation 360

### (R)-2-Amino-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide hydrochloride

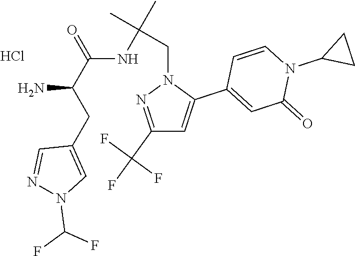 [SMILES: Cl ; CC(C)(Cn1nc(C(F)(F)F)cc1-c1ccn(C2CC2)c(=O)c1)NC(=O)[C@H](N)Cc1cnn(C(F)F)c1]

To tert-butyl (R)-(1-((1-(5-(1-cyclopropyl-2-oxo-1,2-dihydropyridin-4-yl)-3-(trifluoromethyl)-1H-pyrazol-1-yl)-2-methylpropan-2-yl)amino)-3-(1-(difluoromethyl)-1H-pyrazol-4-yl)-1-oxopropan-2-yl)carbamate (9.7 g, 75% wt, 12 mmol) was added DCM (10 mL) and hydrogen chloride in 1,4-dioxane (60 mL, 2 M, 0.12 mol). The reaction was stirred at 25° C. for 2 h, then concentrated under reduced pressure. To the residue was added 40 mL ACN and the mixture was sonicated at 25° C. for 0.5 h. The mixture was filtered, and the filter cake was washed with ACN (10 mL×2), then dried under reduced pressure to give the title compound (5.06 g, 75%) as a white solid. ES-MS m/z 550 (M+Na)⁺.

### Preparation 361

### (6-Chloro-2-methylpyridin-3-yl)methanol

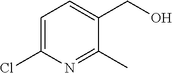

A stirred solution of 6-chloro-2-methylnicotinaldehyde (20.00 g, 128.55 mmol) in THF (400 mL) was cooled to 0° C. and then NaBH₄ (9.7 g, 0.26 mol) was added portion-wise, after which the mixture was warmed to RT. After 6 h, the reaction was cooled to 0° C. and quenched with H₂O. The resulting mixture was stirred overnight, then extracted with EtOAc (3×500 mL). The combined organic layers were washed with sat. aq. NaCl, dried over Na₂SO₄, filtered and concentrated under reduced pressure to obtain the title compound (20.62 g, 98% yield) as a light-yellow solid. ES-MS m/z 158, 160 (M+H).

### Preparation 362

### 1-((6-Chloro-2-methylpyridin-3-yl)methyl)tetrahydro-1H-thiophen-1-ium bromide

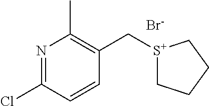

A stirred solution of (6-chloro-2-methylpyridin-3-yl)methanol (20.26 g, 128.6 mmol) in DCM (300 mL) was placed under N₂, cooled to 0° C., and PPh₃ (40.46 g, 154.3 mmol) and CBr₄ (51.16 g, 154.3 mmol) were added. After 3 h, the solution was concentrated under reduced pressure and purified on silica gel using a gradient of 0-15% EtOAc in cyclohexanes, to obtain 3-(bromomethyl)-6-chloro-2-methylpyridine (30.1 g, 96% yield) as a white solid. This was dissolved in acetone (50 mL) under N₂, then tetrahydrothiophene (15 mL, 163.8 mmol) was added, and the reaction was stirred at 25° C. After 3 d, the reaction was diluted with a small amount of EtOAc, and the solids were collected by filtration. The filter cake was washed three times with a small volume of EtOAc and then dried overnight at reduced pressure to obtain the title compound (33.72 g, 80% yield) as a white solid. ES-MS m/z 228 (M⁺).

### Preparation 363

### 1-(4-Bromo-2-methylbenzyl)tetrahydro-1H-thiophen-1-ium bromide

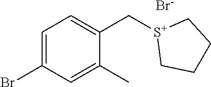

To a solution of 4-bromo-1-(bromomethyl)-2-methylbenzene (10 g, 38 mmol) in HFIP (15 mL) was added tetrahydrothiophene (4 mL, 45 mmol), and the solution was stirred at RT overnight. The reaction mixture was partially concentrated by reduced pressure, then re-suspended in MTBE. The precipitate was rinsed with MTBE and then dried under reduced pressure to obtain the title compound (14 g, 100% yield) as a white powder. ES/MS m/z 273 (M⁺).

### Preparation 364

### Tetrafluoro-24-borane, 1-(4-bromo-2-(trifluoromethyl)benzyl)tetrahydro-1H-thiophen-1-ium salt

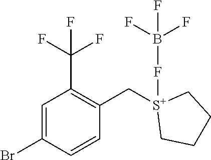

To a suspension of 4-bromo-1-(bromomethyl)-2-(trifluoromethyl)benzene (3 g, 9 mmol) and tetrahydrothiophene (0.8 mL, 9 mmol) in ACN (10 mL) was added silverfluoroborate (2 g, 9 mmol). The reaction was stirred overnight at RT, then filtered over diatomaceous earth. The filtrate was concentrated to give a quantitative yield of the title compound (4.5 g, 90% purity) as an off white solid. ES/MS m/z 325 (M⁺).

### Preparation 365

### (1R,3R)-1-(4-Bromo-2-methylphenyl)-5-oxaspiro[2.4]heptan-4-one

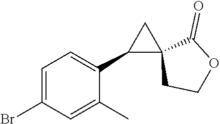 [SMILES: Cc1cc(Br)ccc1[C@H]1C[C@@]12CCOC2=O]

To a solution of 3-methylenedihydrofuran-2(3H)-one (2.7 g, 28 mmol) in DCM (50 mL) was added 1-(4-bromo-2-methylbenzyl)tetrahydro-1H-thiophen-1-ium bromide (7 g, 0.02 mol). The reaction mixture was cooled to 0° C. and lithium bis(trimethylsilyl)amide (1 M in THE, 24 mL, 1.2 mmol) was added dropwise. The reaction mixture was stirred at 0° C. for 30 min then was diluted with sat. aq. ammonium chloride and DCM, then warmed to RT. The aqueous layer was extracted with DCM (3×) and the organic layers were combined, dried over sodium sulfate, and concentrated. The residue was purified by silica gel chromatography (gradient: 0-100% 3:1 EtOAc:EtOH in heptane) followed by chiral SFC purification (Chiralpak IJ 30×250 mm, 5 M, 42 mL/min), eluting with MeOH to afford the title compound (666 mg, 10%) as the first eluting isomer. ES-MS m/z 335, 337 (M+H).

### Preparation 366

### (1R,3R)-1-(6-Chloro-2-methylpyridin-3-yl)-5-oxaspiro[2.4]heptan-4-one

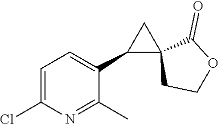 [SMILES: Cc1nc(Cl)ccc1[C@H]1C[C@@]12CCOC2=O]

3-Methylenedihydrofuran-2(3H)-one (12.11 g, 123.4 mmol) in DCM (270 mL) was placed under N₂. 1-((6-Chloro-2-methylpyridin-3-yl)methyl)tetrahydro-1H-thiophen-1-ium bromide (34.63 g, 112.20 mmol) was added, the suspension was cooled to 0° C., and DMSO (270 mL, 112.20 mmol) was added. Then LiHMDS (116 mL, 1.25 M solution in THF, 145 mmol) was added dropwise over 2 h. After 20 min, the reaction was quenched by dilution with EtOAc (300 mL) and ice-cold H₂O (300 mL). The organic layer was removed, and the aq. layer was extracted with EtOAc (2×400 mL). The combined organic layers were washed with H₂O (2×500 mL) and sat. aq. NaCl (500 mL), then dried over Na₂SO₄. The organic layers were filtered and concentrated under reduced pressure to afford a yellow solid which was further concentrated overnight under reduced pressure at 40° C. This material was purified by silica gel chromatography (gradient: 10-30% EtOAc in cyclohexanes) followed by chiral SFC purification (Chiralpak IG, 30×250 mm, 5 μm) eluting with 25% MeOH with 0.5% DMEA to obtain the title compound as the first eluting enantiomer (5.77 g, 20% yield). ES-MS m/z 238 (M+H).

### Preparation 367

### rac-trans-1-(4-Bromo-2-(trifluoromethyl)phenyl)-5-oxaspiro[2.4]heptan-4-one

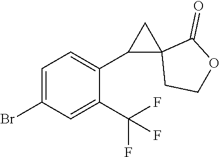 [SMILES: O=C1OCCC12CC2c1ccc(Br)cc1C(F)(F)F]

To a solution of 3-methylenedihydrofuran-2(3H)-one (1 g, 10 mmol) in DCM (32 mL) was added DMSO (2 mL), and tetrafluoro-24-borane, 1-(4-bromo-2-(trifluoromethyl)benzyl)tetrahydro-1H-thiophen-1-ium salt (4 g, 10 mmol) into flask at 25° C. The reaction mixture was cooled to 0° C. and lithium bis(trimethylsilyl)amide (1 M in THF, 13 mL, 1.3 mmol) was added dropwise. The reaction mixture was stirred at 0° C. for 30 min then was diluted with water and DCM and warmed to RT. The aqueous layer was extracted with DCM (3×) and the organic layers were combined, dried over sodium sulfate, and concentrated. The crude product was purified by column chromatography (0-100% 3:1 EtOAc:EtOH in heptane) followed by UPLC CSH (XSelect, C18, 50 mm×250 mm, 5 μm) with 50% ACN/water (+0.1% FA) to obtain the title compound (1.09 g, 33%) as the second eluting isomer. ES-MS m/z 335, 337 (M+H).

### Preparation 368

### (1R,2R)-2-(4-Bromo-2-methylphenyl)-1-(2-chloroethyl)-N—((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)cyclopropane-1-carboxamide

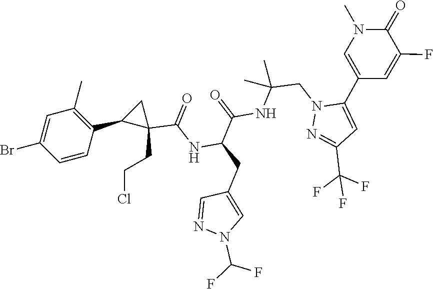 [SMILES: Cc1cc(Br)ccc1[C@H]1C[C@]1(CCCl)C(=O)N[C@H](Cc1cnn(C(F)F)c1)C(=O)NC(C)(C)Cn1nc(C(F)(F)F)cc1-c1cc(F)c(=O)n(C)c1]

A mixture of (1R,3R)-1-(4-bromo-2-methylphenyl)-5-oxaspiro[2.4]heptan-4-one (660 mg, 2.35 mmol) and benzyl (triethyl) ammonium chloride (1.073 g, 4.71 mmol) was suspended in DCE (11.7 mL) under argon. The stirred suspension was cooled to 0° C. and SOCl₂ (690 μL, 9.37 mmol) was added dropwise. After 5 min, BF₃·Et₂O (590 μL, 4.71 mmol) was added dropwise, and then the reaction was heated to 80° C. for 4.5 h. The crude reaction mixture was then placed in a −20° C. freezer for storage overnight. The following day, the reaction was concentrated under reduced pressure to afford the acyl chloride as a pale orange oil. In a separate vessel, (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide hydrochloride (1.45 g, 2.60 mmol) was suspended in DCM (10 mL) under argon. Then TEA (2.0 mL, 14 mmol) was added, and the resulting suspension was cooled to 0° C. Separately, the crude acyl chloride was dissolved in DCM (5 mL). This solution was added dropwise to the former suspension, using additional DCM (2×2.5 mL) to aid transfer. The reaction was halted after 25 min by dilution with 1 M aq. HCl (75 mL), sat. aq. NaCl (75 mL), and DCM (30 mL). The organic layer was removed, and the aq. layer was extracted with DCM (4×30 mL). The combined organic layers were then dried over Na₂SO₄, filtered, and concentrated via reduced pressure. The crude material was then purified 2× on silica gel using a gradient of 0-80% 3:1 EtOAc:EtOH in heptane to obtain the title compound (831.9 mg, 41% yield) as a foamy white solid. ES-MS m/z 818, 820 (M+H).

### Preparation 369

### (1R,2R)-2-(6-Chloro-2-methylpyridin-3-yl)-1-(2-chloroethyl)-N—((R)-1-((1-(3-(1-cyclopropyl-2-oxo-1,2-dihydropyridin-4-yl)-5-(trifluoromethyl)-1H-pyrazol-1-yl)-2-methylpropan-2-yl)amino)-3-(1-(difluoromethyl)-1H-pyrazol-4-yl)-1-oxopropan-2-yl)cyclopropane-1-carboxamide

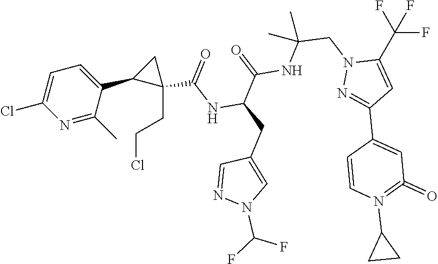

To a mixture of (1R,3R)-1-(6-chloro-2-methylpyridin-3-yl)-5-oxaspiro[2.4]heptan-4-one (1.21 g, 5.07 mmol) and benzyltriethylammonium chloride (1.78 g, 7.82 mmol) was added DCE (16 mL). The vial was sealed and purged with N₂ and then treated with SOCl₂ (950 μL, 13.0 mmol) at 0° C., followed by BF₃·Et₂O (800 μL, 6.48 mmol). The reaction mixture was heated to 80° C. overnight, then was cooled to RT and concentrated under reduced pressure to obtain a quantitative yield of (1R,2R)-2-(6-chloro-2-methylpyridin-3-yl)-1-(2-chloroethyl)cyclopropane-1-carbonyl chloride as a yellow sticky solid. In a separate vessel, a mixture of (R)-2-amino-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide hydrochloride (2.65 g, 4.7 mmol) and TEA (4.5 mL, 32 mmol) in DCM (10.0 mL) was stirred at 0° C. A suspension of (1R,2R)-2-(6-chloro-2-methylpyridin-3-yl)-1-(2-chloroethyl)cyclopropane-1-carbonyl chloride (1.48 g, 5.07 mmol) in DCM (10 mL) was added dropwise. The cooling bath was removed, and the reaction mixture was stirred at RT for 0.5 h. The crude was purified by silica gel chromatography eluting with 0-60% EtOAc:EtOH (3:1) in heptane to obtain title compound (3.52 g, 80%) as an off-white solid. ES-MS m/z 784 (M+H).

The compounds in the following table were prepared as described in Preparation 369 using the appropriate lactone and appropriate amine or amine salt. Reaction time may vary. Slight variations to the reaction conditions and purification methods would be apparent to one skilled in the art.

TABLE 26

| | | | ES-MS |
|---|---|---|---|
| Preparation | Chemical Name | Structure | m/z |
| 370^{a} | Methyl (R)-2-((1R,2S)-2-(4- bromo-2-chlorophenyl)-1-(2- chloroethyl)cyclopropane-1- carboxamido)-3-(1- (difluoromethyl)-1H-pyrazol- 4-yl)propanoate | 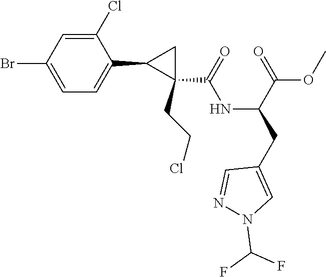 [SMILES: COC(=O)[C@@H](Cc1cnn(C(F)F)c1)NC(=O)[C@@]1(CCCl)C[C@@H]1c1ccc(Br)cc1Cl] | 540 (M + H) |
| 371^{b} | (trans)-2-(4-Bromo-2- (trifluoromethyl)phenyl)-1- (2-chloroethyl)-N-((R)-3-(1- (difluoromethyl)-1H-pyrazol- 4-yl)-1-((2-methyl-1-(3-(1- methyl-6-oxo-1,6- dihydropyridin-3-yl)-5- (trifluoromethyl)-1H- pyrazol-1-yl)propan-2- yl)amino)-1-oxopropan-2- yl)cyclopropane-1- carboxamide (mixture of isomer) | 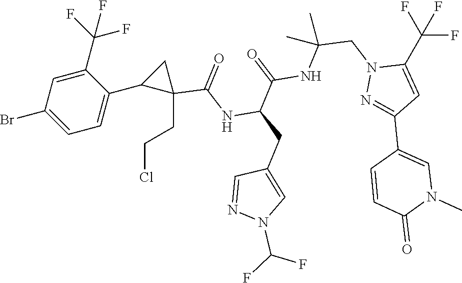 [SMILES: Cn1cc(-c2cc(C(F)(F)F)n(CC(C)(C)NC(=O)[C@@H](Cc3cnn(C(F)F)c3)NC(=O)C3(CCCl)CC3c3ccc(Br)cc3C(F)(F)F)n2)ccc1=O] | 856 (M + H) |

^{a}Acylation was carried out in THF at a final concentration of 0.1 M with TEA (8 equivalents) as base.
^{b}Acylation was carried out in DCM at a final concentration of 0.2 M with TEA (6 equivalents) as base.

### Preparation 372

### (R)-2-((1R,3R)-1-(4-Bromo-2-methylphenyl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide

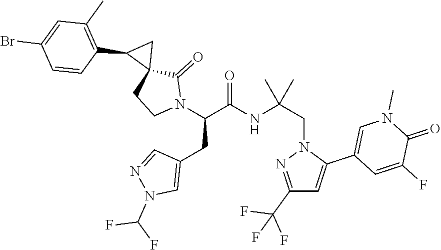 [SMILES: Cc1cc(Br)ccc1[C@H]1C[C@@]12CCN([C@H](Cc1cnn(C(F)F)c1)C(=O)NC(C)(C)Cn1nc(C(F)(F)F)cc1-c1cc(F)c(=O)n(C)c1)C2=O]

(1R,2R)-2-(4-Bromo-2-methylphenyl)-1-(2-chloroethyl)-N—((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)cyclopropane-1-carboxamide (832 mg, 0.97 mmol), TBAI (40.3 mg, 110 μmol), and Cs₂CO₃ (668 mg, 2.05 mmol) were combined in NMP (10.0 mL). After 17.5 h, the reaction was quenched by dilution with EtOAc and sat. aq. NaCl. The aq. layer was removed, and the organic layer was washed 5× with sat. aq. NaCl. The organic layer was then dried over Na₂SO₄, filtered, concentrated under reduced pressure, and purified on silica gel using a gradient of 0-80% 3:1 EtOAc:EtOH in heptane. This material was combined with a second batch from a smaller scale reaction (0.17 mmol scale) and concentrated under reduced pressure to obtain the title compound (786.8 mg, 81% purity, 71%) as a yellow oil. ES-MS m/z 782, 784 (M+H).

### Preparation 373

### (R)-2-((1R,3R)-1-(6-Chloro-2-methylpyridin-3-yl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide

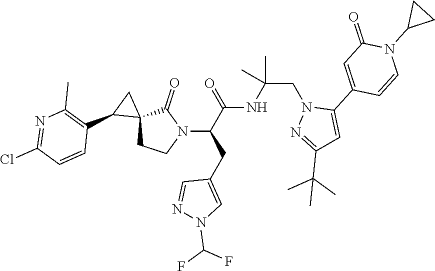 [SMILES: Cc1nc(Cl)ccc1[C@H]1C[C@@]12CCN([C@H](Cc1cnn(C(F)F)c1)C(=O)NC(C)(C)Cn1nc(C(C)(C)C)cc1-c1ccn(C3CC3)c(=O)c1)C2=O]

A mixture of (1R,2R)-2-(6-chloro-2-methylpyridin-3-yl)-1-(2-chloroethyl)-N—((R)-1-((1-(3-(1-cyclopropyl-2-oxo-1,2-dihydropyridin-4-yl)-5-(trifluoromethyl)-1H-pyrazol-1-yl)-2-methylpropan-2-yl)amino)-3-(1-(difluoromethyl)-1H-pyrazol-4-yl)-1-oxopropan-2-yl)cyclopropane-1-carboxamide (3.52 g, 4.49 mmol) in NMP (15 mL) was added Cs₂CO₃ (3.5 g, 10.6 mmol). The resulting mixture was stirred vigorously overnight at RT. The crude mixture was partitioned between H₂O (50 mL) and EtOAc (50 mL). The aqueous layer was extracted with EtOAc (2×50 mL), and the combined organic fractions were washed with sat. aq. NaCl (3× 50 mL), dried over anhydrous Na₂SO₄, filtered and concentrated under reduced pressure. The crude product was purified by silica gel chromatography eluting with 0-100% heptane/EtOAc: EtOH (3:1) to obtain title compound (2.65 g, 72%) as a pink solid. ES-MS m/z 747 (M+H).

The compounds in the following table were prepared as described in Preparation 373. ACN is a suitable replacement for NMP. Reaction times and temperatures may vary, and differing methods can be used to work up or purify the compounds (normal phase, or high, neutral or low pH reversed phase). Such variances would be apparent to one skilled in the art.

TABLE 27

| | | | ES-MS |
|---|---|---|---|
| Preparation | Chemical Name | Structure | m/z |
| 374ª | Methyl (R)-2-((1S,3R)-1- (4-bromo-2- chlorophenyl)-4-oxo-5- azaspiro[2.4]heptan-5-yl)- 3-(1-(difluoromethyl)-1H- pyrazol-4-yl)propanoate | 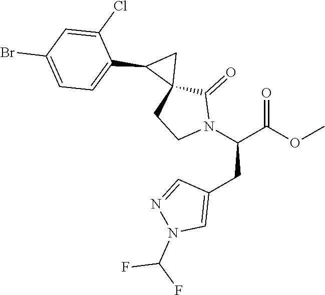 [SMILES: COC(=O)[C@@H](Cc1cnn(C(F)F)c1)N1CC[C@@]2(C[C@@H]2c2ccc(Br)cc2Cl)C1=O] | 502, 504 (M + H) |
| 375ª | (R)-2-((trans)-1-(4- Bromo-2- (trifluoromethyl)phenyl)- 4-oxo-5- azaspiro[2.4]heptan-5-yl)- 3-(1-(difluoromethyl)-1H- pyrazol-4-yl)-N-(2- methyl-1-(5-(1-methyl-6- oxo-1,6-dihydropyridin-3- yl)-3-(trifluoromethyl)- 1H-pyrazol-1-yl)propan-2- yl)propanamide (mixture of isomers) | 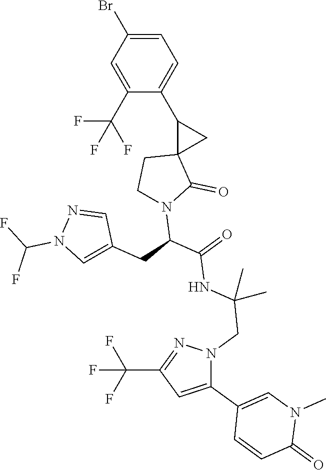 [SMILES: Cn1cc(-c2cc(C(F)(F)F)nn2CC(C)(C)NC(=O)[C@@H](Cc2cnn(C(F)F)c2)N2CCC3(CC3c3ccc(Br)cc3C(F)(F)F)C2=O)ccc1=O] | 818, 820 (M + H) |

^{a}No TBAI was added

### Preparation 376

### (1S,2S)-2-(3-Chloro-6-cyano-1-methyl-1H-pyrazolo[4,3-b]pyridin-5-yl)cyclopropane-1-carboxylic acid

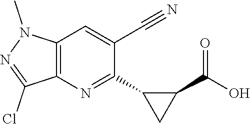 [SMILES: Cn1nc(Cl)c2nc([C@H]3C[C@@H]3C(=O)O)c(C#N)cc21]

To ethyl (1S,2S)-2-(trifluoro-24-boraneyl)cyclopropane-1-carboxylate, potassium salt (291.2 mg, 1.32 mmol) was added 3,5-dichloro-1-methyl-1H-pyrazolo[4,3-b]pyridine-6-carbonitrile (315.6 mg, 1.39 mmol), Cs₂CO₃ (1.29 g, 3.96 mmol) and Catacxium Pd G4 (128.3 mg, 173 μmol). Toluene (4 mL) and water (1 mL) were added the resulting mixture was purged under N₂ for 3 min. The reaction was heated to 110° C. for 3.5 h then allowed to cool to RT overnight. The cooled crude mixture was partitioned between water (20 mL) and EtOAc (25 mL). The separated aqueous layer was extracted with EtOAc (25 mL×2). The combined organic layers were washed with sat. aq. NaCl (25 mL), dried over Na₂SO₄, filtered, and concentrated under reduced pressure. The crude product was purified on silica gel, eluting with EtOAc:EtOH (3:1) (0-100%) in heptane, followed by a second purification on silica gel eluting with EtOAc (0-100%) in heptane to obtain ethyl (1S,2S)-2-(3-chloro-6-cyano-1-methyl-1H-pyrazolo[4,3-b]pyridin-5-yl)cyclopropane-1-carboxylate (277.4 mg). ES/MS ES-MS m/z 305 (M+H). To a vial containing ethyl (1S,2S)-2-(3-chloro-6-cyano-1-methyl-1H-pyrazolo[4,3-b]pyridin-5-yl)cyclopropane-1-carboxylate (277.4 mg, 910.3 μmol) was added 2.0 M aq. LiOH (5.5 mL, 11 mmol), THF (2 mL) and MeOH (2 mL). The resulting mixture was stirred at RT overnight. To the reaction mixture was then added 4.0 M HCl in 1,4-dioxane (2.5 mL, 4.0 molar, 10 mmol) and stirred for 5 mins, and then concentrated under steam of nitrogen to obtain a quantitative yield of the title compound (777.4 mg, 34% purity) as an off-white solid. ES-MS m/z 277 (M+H).

The compounds in the following table were prepared as described in Preparation 376 using the appropriate aryl halide coupling partners. Various methods were used to purify the compounds, which would be apparent to one skilled in the art.

TABLE 28

| Preparation | Chemical Name | Structure | ES-MS m/z |
|---|---|---|---|
| 377 | (1S,2S)-2-(6-Fluoro-1- methyl-1H-pyrazolo[4,3- b]pyridin-5- yl)cyclopropane-1- carboxylic acid | 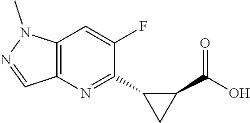 [SMILES: Cn1ncc2nc([C@H]3C[C@@H]3C(=O)O)c(F)cc21] | 236 (M + H) |
| 378 | (1S,2S)-2-(5-Fluoro-1- methyl-1H-pyrazolo[3,4- b]pyridin-6- yl)cyclopropane-1- carboxylic acid | 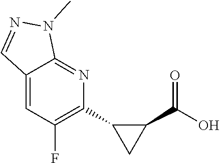 [SMILES: Cn1ncc2cc(F)c([C@H]3C[C@@H]3C(=O)O)nc21] | 236 (M + H) |

### Preparation 379

### (R)-2-((1S,3R)-1-(4-Bromo-2-chlorophenyl)-4-oxo-5-azaspiro[2.4]heptan-5-yl)-3-(1-(difluoromethyl)-1H-pyrazol-4-yl)propanoic acid

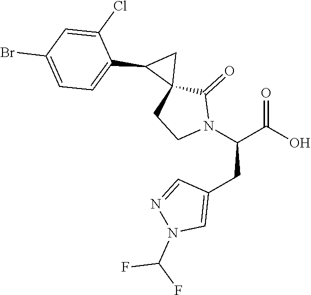 [SMILES: O=C(O)[C@@H](Cc1cnn(C(F)F)c1)N1CC[C@@]2(C[C@@H]2c2ccc(Br)cc2Cl)C1=O]

To a solution of methyl (R)-2-((1S,3R)-1-(4-bromo-2-chlorophenyl)-4-oxo-5-azaspiro[2.4]heptan-5-yl)-3-(1-(difluoromethyl)-1H-pyrazol-4-yl)propanoate (4.97 g, 9.79 mmol) in tert-butanol (60 mL) and H₂O (30 mL) was added 1 M aq. LiOH (30 mL, 30 mmol) at 0° C. The reaction mixture was stirred at 0° C. for 2 h. The mixture was adjusted to pH=2 with 1 Naq. HCl, diluted with H₂O (100 mL), and extracted with EtOAc (100 mL×2). The combined organic layers were washed with sat. aq. NaCl (80 mL×2), dried over Na₂SO₄, filtered, and concentrated under reduced pressure to give a residue (˜4.6 g) as a yellow solid. 2.5 g of the residue was purified by silica gel chromatography eluting with 0-3% MeOH/DCM to give the title compound (2.0 g, 42%) as a white solid. ES-MS m/z 488, 490 (M+H).

### Preparation 380

### (R)-2-((1S,3R)-1-(4-Bromo-2-fluorophenyl)-4-oxo-5-azaspiro[2.4]heptan-5-yl)-3-(1-(difluoromethyl)-1H-pyrazol-4-yl)propanoic acid

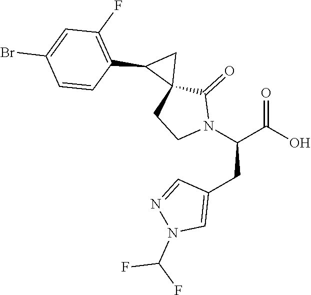 [SMILES: O=C(O)[C@@H](Cc1cnn(C(F)F)c1)N1CC[C@@]2(C[C@@H]2c2ccc(Br)cc2F)C1=O]

The title compound was prepared as described in Preparation 379 using methyl (R)-2-((1S,3R)-1-(4-bromo-2-fluorophenyl)-4-oxo-5-azaspiro[2.4]heptan-5-yl)-3-(1-(difluoromethyl)-1H-pyrazol-4-yl)propanoate (2.25 g, 4.40 mmol). ES-MS m/z 472, 474 (M+H).

### Preparation 381

### (R)-2-((1S,3R)-1-(4-Bromo-2-chlorophenyl)-4-oxo-5-azaspiro[2.4]heptan-5-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide

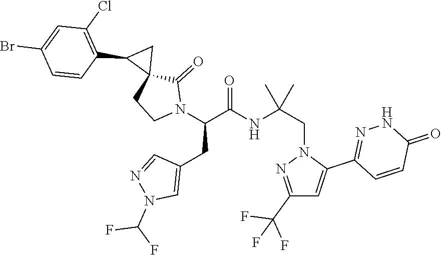 [SMILES: CC(C)(Cn1nc(C(F)(F)F)cc1-c1ccc(=O)[nH]n1)NC(=O)[C@@H](Cc1cnn(C(F)F)c1)N1CC[C@@]2(C[C@@H]2c2ccc(Br)cc2Cl)C1=O]

To a mixture of (R)-2-((1S,3R)-1-(4-bromo-2-chlorophenyl)-4-oxo-5-azaspiro[2.4]heptan-5-yl)-3-(1-(difluoromethyl)-1H-pyrazol-4-yl)propanoic acid (1.01 g, 1.98 mmol) and 6-(1-(2-amino-2-methylpropyl)-3-(trifluoromethyl)-1H-pyrazol-5-yl)pyridazin-3 (2H)-one dihydrochloride (810 mg, 1.95 mmol) was added DMA (25 mL), diethyl (4-oxo-1,2,3-benzotriazin-3-yl)phosphate (1.15 g, 3.84 mmol) and 4-methylmorpholine (2.25 mL, 20 mmol). The resulting solution was allowed to stir at 20° C. for 2 h, then was diluted with H₂O and extracted with EtOAc. The combined organic layers were washed with sat. aq. NaCl, dried over anhydrous Na₂SO₄, filtered, and concentrated under reduced pressure. The residue was purified by silica gel chromatography eluting with 0-4% MeOH/DCM to give the title compound (1.31 g, 81%) as a white solid. ES-MS m/z 773 (M+H).

### Preparation 382

### (R)-2-((1S,3R)-1-(4-Bromo-2-fluorophenyl)-4-oxo-5-azaspiro[2.4]heptan-5-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide

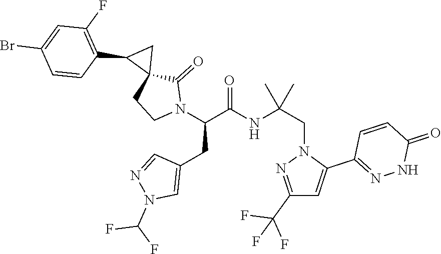 [SMILES: CC(C)(Cn1nc(C(F)(F)F)cc1-c1ccc(=O)[nH]n1)NC(=O)[C@@H](Cc1cnn(C(F)F)c1)N1CC[C@@]2(C[C@@H]2c2ccc(Br)cc2F)C1=O]

The title compound was prepared as described in Preparation 381 using (R)-2-((1S,3R)-1-(4-bromo-2-fluorophenyl)-4-oxo-5-azaspiro[2.4]heptan-5-yl)-3-(1-(difluoromethyl)-1H-pyrazol-4-yl)propanoic acid and 6-(1-(2-amino-2-methylpropyl)-3-(trifluoromethyl)-1H-pyrazol-5-yl)pyridazin-3 (2H)-one hydrochloride (1.1 g, 2.9 mmol), stirring for 12 h. ES-MS m z 755, 757 (M+H).

### Preparation 383

### (2R)-2-((1S,3R)-1-(4-Bromo-2-chlorophenyl)-4-oxo-5-azaspiro[2.4]heptan-5-yl)-3-(1-(difluoromethyl)-1H-pyrazol-4-yl)-N-(2-methyl-1-(5-(2-(tetrahydro-2H-pyran-2-yl)-2H-1,2,3-triazol-4-yl)-3-(trifluoromethyl)-1H-pyrazol-1-yl)propan-2-yl)propanamide

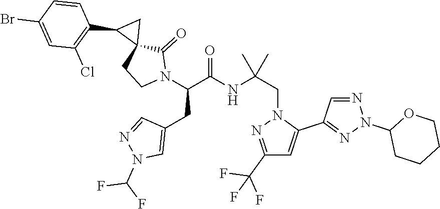 [SMILES: CC(C)(Cn1nc(C(F)(F)F)cc1-c1cnn(C2CCCCO2)n1)NC(=O)[C@@H](Cc1cnn(C(F)F)c1)N1CC[C@@]2(C[C@@H]2c2ccc(Br)cc2Cl)C1=O]

To a mixture of (R)-2-((1S,3R)-1-(4-bromo-2-chlorophenyl)-4-oxo-5-azaspiro[2.4]heptan-5-yl)-3-(1-(difluoromethyl)-1H-pyrazol-4-yl)propanoic acid (195 mg, 383 μmol), 2-methyl-1-(5-(2-(tetrahydro-2H-pyran-2-yl)-2H-1,2,3-triazol-4-yl)-3-(trifluoromethyl)-1H-pyrazol-1-yl)propan-2-amine hydrochloride (292 mg, 385 μmol) and DIEA (250 μL, 1.44 mmol) in DMA (4 mL) was added HATU (220 mg, 579 μmol). The reaction mixture was stirred at 20° C. for 1 h, then additional (R)-2-((1R,3S)-1-(4-bromo-2-chlorophenyl)-4-oxo-5-azaspiro[2.4]heptan-5-yl)-3-(1-(difluoromethyl)-1H-pyrazol-4-yl)propanoic acid (45 mg, 88 μmol) was added. The reaction mixture was stirred for 2 h, diluted with H₂O (20 mL) and extracted with EtOAc (20 mL×3). The combined organic layers were washed with sat. aq. NaCl (20 mL×3), dried over anhydrous Na₂SO₄, filtered, and concentrated under reduced pressure. The crude product was purified by silica gel chromatography (0-4% MeOH in DCM) to provide the title compound (298 mg, 62% purity, 58%) as a colorless oil. ES-MS m/z 830 (M+H).

### Preparation 384

### (2R)-2-((1S,3R)-1-(4-Bromo-2-chlorophenyl)-4-oxo-5-azaspiro[2.4]heptan-5-yl)-3-(1-(difluoromethyl)-1H-pyrazol-4-yl)-N-(2-methyl-1-(1-(1-methyl-2-oxopyrrolidin-3-yl)-4-(trifluoromethyl)-1H-imidazol-2-yl)propan-2-yl)propanamide

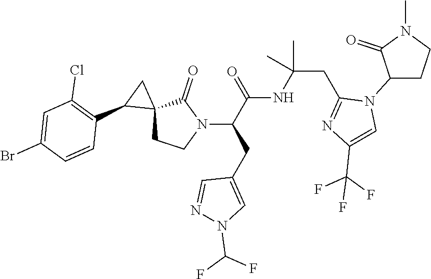 [SMILES: CN1CCC(n2cc(C(F)(F)F)nc2CC(C)(C)NC(=O)[C@@H](Cc2cnn(C(F)F)c2)N2CC[C@@]3(C[C@@H]3c3ccc(Br)cc3Cl)C2=O)C1=O]

To (R)-2-((1S,3R)-1-(4-bromo-2-chlorophenyl)-4-oxo-5-azaspiro[2.4]heptan-5-yl)-3-(1-(difluoromethyl)-1H-pyrazol-4-yl)propanoic acid (202.6 mg, 414.6 μmol) was added 3-(2-(2-amino-2-methylpropyl)-4-(trifluoromethyl)-1H-imidazol-1-yl)-1-methylpyrrolidin-2-one hydrochloride (racemic mixture, 190.5 mg, 559.0 μmol) and DMA (2.0 mL). The solution was then treated with 4-methylmorpholine (0.23 mL, 2.1 mmol) and cooled to 0° C. in an ice bath. Diethyl (4-oxo-1,2,3-benzotriazin-3-yl)phosphate (251.0 mg, 838.8 μmol) was added and the reaction mixture was allowed to gradually warm to RT overnight. The reaction mixture was partitioned between EtOAc (25 mL) and H₂O (15 mL) and the aqueous layer was extracted with EtOAc (25 mL). The combined organic layers were washed with sat. aq. NaCl (25 mL), dried over Na₂SO₄, and concentrated under reduced pressure. Column chromatography (SiO₂, gradient 0-100% EtOAc in heptane, then 25% EtOH in EtOAc), provided the title compound as a sticky yellow gel. ES-MS m/z 774.2 (M+H).

### Preparation 385

### (4-((trans)-5-((R)-3-(1-(Difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-4-oxo-5-azaspiro[2.4]heptan-1-yl)-3-(trifluoromethyl)phenyl) boronic acid (mixture of isomers)

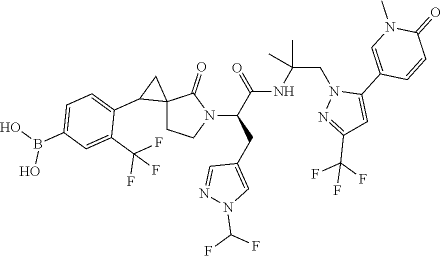 [SMILES: Cn1cc(-c2cc(C(F)(F)F)nn2CC(C)(C)NC(=O)[C@@H](Cc2cnn(C(F)F)c2)N2CCC3(CC3c3ccc(B(O)O)cc3C(F)(F)F)C2=O)ccc1=O]

To a mixture of (R)-2-((trans)-1-(4-bromo-2-(trifluoromethyl)phenyl)-4-oxo-5-azaspiro[2.4]heptan-5-yl)-3-(1-(difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)propanamide (mixture of isomers, 661 mg, 808 μmol) and bis(pinacolato)diboron (639.0 mg, 2.52 mmol) was added Pd(dppf)Cl₂ (DCM adduct, 104.4 mg, 127.8 μmol), KOAc (246.1 mg, 2.51 mmol) and 1,4-dioxane (6 mL). The resulting solution was purged with N₂ for 3 min then heated to 80° C. overnight. The reaction mixture was cooled to RT to give a solution of the title compound that was used directly in the next step. ES-MS m/z 784 (M+H).

### Preparation 386

### (2R)-2-((1S,3R)-1-(2-Chloro-4-(1-methyl-2-oxo-1,2-dihydropyrimidin-5-yl)phenyl)-4-oxo-5-azaspiro[2.4]heptan-5-yl)-3-(1-(difluoromethyl)-1H-pyrazol-4-yl)-N-(2-methyl-1-(5-(2-(tetrahydro-2H-pyran-2-yl)-2H-1,2,3-triazol-4-yl)-3-(trifluoromethyl)-1H-pyrazol-1-yl)propan-2-yl)propanamide

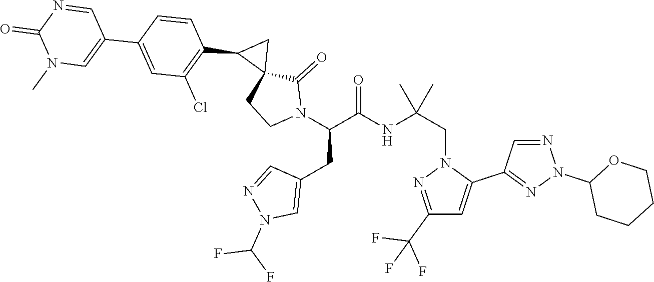 [SMILES: Cn1cc(-c2ccc([C@H]3C[C@@]34CCN([C@H](Cc3cnn(C(F)F)c3)C(=O)NC(C)(C)Cn3nc(C(F)(F)F)cc3-c3cnn(C5CCCCO5)n3)C4=O)c(Cl)c2)cnc1=O]

A mixture of (2R)-2-((1S,3R)-1-(4-bromo-2-chlorophenyl)-4-oxo-5-azaspiro[2.4]heptan-5-yl)-3-(1-(difluoromethyl)-1H-pyrazol-4-yl)-N-(2-methyl-1-(5-(2-(tetrahydro-2H-pyran-2-yl)-2H-1,2,3-triazol-4-yl)-3-(trifluoromethyl)-1H-pyrazol-1-yl)propan-2-yl)propanamide (150 mg, 112 μmol), 1-methyl-5-(tributylstannyl)pyrimidin-2(1H)-one (78 mg, 0.18 mmol), CsF (37 mg, 0.24 mmol) and chloro[(di(1-adamantyl)-N-butylphosphine)-2-(2-aminobiphenyl)]palladium (II) (20 mg, 30 μmol) in 1,4-dioxane (2.5 mL) was degassed and backfilled with N₂ three times. The mixture was then stirred at 100° C. for 12 h. The reaction mixture was filtered and the filter cake was washed with EtOAc (5 mL×3). The filtrate was concentrated under reduced pressure then purified on silica gel (gradient 0-3% MeOH in DCM) to provide the title compound (105 mg, 54% purity, 59%) as a yellow solid. ES-MS m/z 858 (M+H).

### Preparation 387

### 3-Benzoylpyrimidine-2,4 (1H,3H)-dione

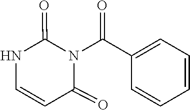 [SMILES: O=C(c1ccccc1)n1c(=O)cc[nH]c1=O]

To a mixture of pyrimidine-2,4 (1H,3H)-dione (14.8 g, 132 mmol) in ACN (132 mL) and pyridine (56 mL) was added dropwise benzoyl chloride (35 mL, 0.30 mol) at 16° C. The reaction mixture was stirred at 16° C. for 32 h then was concentrated. The residue was diluted with water (400 mL), then extracted with DCM (300 mL×2). The combined organic layers were concentrated, and the resulting crude product was further purified by trituration with DCM/Hexanes (v/v=1:1, 100 mL) at 25° C. for 16 h. The solids were filtered and dried under reduced pressure to give the title compound (24.1 g, 76%) as a white solid. ES-MS m/z 215.1 (M-1).

### Preparation 388

### 3-benzoyl-1-cyclopropylpyrimidine-2,4 (1H,3H)-dione

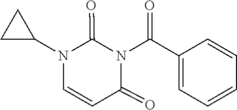 [SMILES: O=C(c1ccccc1)n1c(=O)ccn(C2CC2)c1=O]

To a solution of 3-benzoylpyrimidine-2,4 (1H,3H)-dione (24.1 g, 100 mmol) and cyclopropylboronic acid (21.1 g, 246 mmol) in DCE (450 mL) was added copper (II) acetate (17.9 g, 98.6 mmol), 2,2′-bipyridine (16.1 g, 1.03 Eq, 103 mmol) and Na₂CO₃ (22 g, 0.21 mol). The reaction mixture was degassed and refilled with O₂ (3×) at 20° C., then was stirred at 75° C. under O₂ (15 psi) for 16 h. EtOAc (500 mL) was added, and the mixture was filtered through a pad of celite, washing with EtOAc (400 mL). The filtrate was concentrated under reduced pressure to give a green oil that was further purified by trituration with EtOAc/MeOH/Hexanes (v: v: v=1:1:1, 150 mL) at 20° C. for 0.5 h. The solids were filtered and dried under reduced pressure to give the title compound (1.5 g, 5.3%) as a white solid. ES-MS m/z 257.0 (M+H).

### Preparation 389

### 4-chloro-1-cyclopropylpyrimidin-2(1H)-one

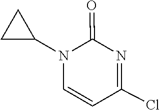 [SMILES: O=c1nc(Cl)ccn1C1CC1]

A suspension of 3-benzoyl-1-cyclopropylpyrimidine-2,4 (1H,3H)-dione (1.49 g, 5.23 mmol) in phosphoryl trichloride (12 mL, 0.13 mol) was degassed and purged with N₂ (3×) at 20° C. The reaction mixture was heated to 70° C. and allowed to stir for 16 h. The reaction mixture was then concentrated under reduced pressure to give a dark oil, which was diluted with DCM (30 mL). The resulting solution was added slowly to ice-cold saturated aq. NaHCO₃ (40 mL). The organic phase was separated, dried over anhydrous Na₂SO₄, and filtered. The filtrate was concentrated under reduced pressure to give the residue as brown solid which was triturated with DCM/Hexanes (v/v=1:10, 20 mL) at 20° C. for 0.5 h. The mixture was filtered and the solids were dried under reduced pressure to the title compound (465 mg, 42%, 80% Purity) as a yellow solid. ES-MS m/z 170.9, 172.9 (M+H)

### Preparation 390

### 5-bromo-2-cyclopropyl-2H-pyrazolo[3,4-b]pyridine

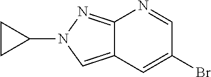 [SMILES: Brc1cnc2nn(C3CC3)cc2c1]

To a solution of 5-bromo-2-nitronicotinaldehyde (414 mg, 1.70 mmol) in IPA (8 mL) was added cyclopropylamine (355 μL, 5.11 mmol). The mixture was bubbled with N₂, then stirred at 40° C. for 3 h. The solution was cooled to 20° C., then tributyl phosphine (1.3 mL, 5.2 mmol) was added, and the mixture was further degassed and bubbled with N₂. The mixture was stirred at 80° C. for 16 h, then was diluted with H₂O (50 mL) and extracted with EtOAc (50 mL×2). The combined organic layers were washed with sat. aq. NaCl (30 mL), dried over Na₂SO₄, filtered, and concentrated under reduced pressure. The crude product was purified on silica gel, eluting with 26% THF in hexanes to provide the title compound (63 mg, 15%) as a white solid. ES-MS m/z 237.9, 239.9 (M+H).

### Preparation 391

### 5-bromo-2-ethyl-2H-pyrazolo[3,4-b]pyridine

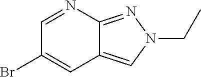

5-bromo-2H-pyrazolo[3,4-b]pyridine (4.80 g, 24.2 mmol) in THF (50 mL) and DMA (50 mL) was cooled to 0° C. and potassium tert-butoxide (1.0 M in THF, 30 mL, 30 mmol) was added dropwise, followed by ethyl iodide (4.0 mL, 49 mmol). Upon completion, the reaction mixture was stirred at 0° C. under Ar for 70 min. The solution was diluted with EtOAc (300 mL), then washed sequentially with water (300 mL) and sat. aq. NaCl (300 mL), dried over Na₂SO₄, filtered, and concentrated under reduced pressure. The crude product was purified on silica, eluting with 0-100% EtOAc in heptane to provide the title compound (836 mg, 15%) as a brown crystalline solid. ES-MS m/z 226.0/228.0 (M+H).

### Preparation 392

### 2-ethyl-5-(4,4,5,5-tetramethyl-1,3,2-dioxaborolan-2-yl)-2H-pyrazolo[3,4-b]pyridine

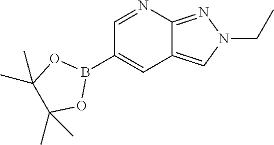 [SMILES: CCn1cc2cc(B3OC(C)(C)C(C)(C)O3)cnc2n1]

5-bromo-2-ethyl-2H-pyrazolo[3,4-b]pyridine (215 mg, 953 μmol) was charged with bis(pinacolato)diboron (596 mg, 2.35 mmol), KOAc (277.9 mg, 2.832 mmol), PdCl₂dppf CH₂Cl₂ (79.7 mg, 97.6 μmol) and 1,4-dioxane (4 mL). The mixture was purged with N₂ for 3 min and then heated to 80° C. under N₂. The reaction mixture was cooled to RT to obtain a quantitative yield of the title compound. ES-MS m/z 274.2 (M+H).

### Preparation 393

### (2-(methoxymethyl) imidazo[1,2-a]pyrimidin-6-yl) boronic acid

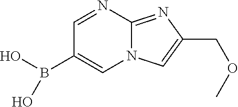

6-Bromo-2-(methoxymethyl) imidazo[1,2-a]pyrimidine (1.99 g, 8.22 mmol), bis(pinacolato)diborane (6.42 g, 25.3 mmol), KOAc (2.420 g, 24.7 mmol), and Pd(dppf)Cl₂·CH₂Cl₂ (1.0 g, 1.2 mmol) were combined in 1,4-dioxane (40 mL) and the mixture was purged with N₂ for 15 min before stirring the mixture at 80° C. overnight. The mixture was concentrated under reduced pressure, and the resulting residue was redissolved in EtOAc and filtered through a short pad of celite. The filtrate was washed with water, sat. aq. NaCl, dried over Na₂SO₄ and concentrated to give the title compound as a purple solid (1.4 g, 83.45%). ES-MS m/z 208.0 (M+H)

### Preparation 394

### 2-cyclopropyl-5-(4,4,5,5-tetramethyl-1,3,2-dioxaborolan-2-yl)-2H-pyrazolo[3,4-b]pyridine

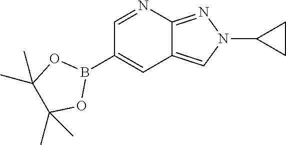 [SMILES: CC1(C)OB(c2cnc3nn(C4CC4)cc3c2)OC1(C)C]

A mixture of 5-bromo-2-cyclopropyl-2H-pyrazolo[3,4-b]pyridine (63.0 mg, 0.25 mmol), 4,4,4′,4′,5,5,5′,5′-octamethyl-2,2′-bi (1,3,2-dioxaborolane) (101 mg, 398 μmol), KOAc (61.0 mg, 0.62 mmol) and Pd(dppf)Cl₂·CH₂Cl₂ (26.0 mg, 32 μmol) in 1,4-dioxane (3 mL) was degassed and purged with N₂ (3×), then stirred at 90° C. for 16 h under N₂. The reaction mixture was then combined with crude material from another reaction (0.15 mmol scale), diluted with EtOAc (10 mL), filtered, and the filter cake was washed with EtOAc. The filtrate was concentrated under reduced pressure to give a residue which was diluted with hexanes (3 mL) and filtered. The filter cake was concentrated under reduced pressure to provide the title compound (156 mg, 88%) as a brown solid. ES-MS m/z 203.9 (M-pinacol+H).

### Preparation 395

### ethyl (1S,2S)-2-(6-fluoro-3-methyl-2-oxo-2,3-dihydrobenzo[d]oxazol-5-yl)cyclopropane-1-carboxylate

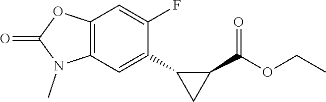 [SMILES: CCOC(=O)[C@H]1C[C@@H]1c1cc2c(cc1F)oc(=O)n2C]

To ethyl (1S,2S)-2-(trifluoro-14-boraneyl)cyclopropane-1-carboxylate, potassium salt (180.0 mg, 818.0 μmol) was added 5-bromo-6-fluoro-3-methylbenzo[d]oxazol-2(3H)-one (152.9 mg, 621.4 μmol), Cs₂CO₃ (603.7 mg, 1.853 mmol), catacxium Pd G4 (50.3 mg, 67.8 μmol), toluene (4 mL) and water (1 mL). The resulting mixture was purged under N₂ for 5 min, then stirred at 110° C. for 2 h. The cooled reaction mixture was partitioned between water (15 mL) and EtOAc (25 mL) and the aqueous layer was extracted with EtOAc (15 mL×2). The organic layers were combined, washed with sat. aq. NaCl, dried over Na₂SO₄, filtered, and concentrated under reduced pressure. The residue was purified on silica, eluting with 0-60% (3:1 EtOAc: EtOH) in hexane to obtain the title compound (174.1 g, 98%) as brown fluffy powder. ES-MS m/z 280.0 (M+H).

The compounds in the following table were prepared as described in Preparation 395 using the appropriate aryl bromide. Reactants can be added in different orders or in differing equivalency and differing methods can be used to work up or purify the compounds (normal phase, or high, neutral or low pH reversed phase). Such variances would be apparent to one skilled in the art.

TABLE 43

| Preparation | Chemical Name | Structure | ES-MS m/z |
|---|---|---|---|
| 396ª | ethyl (1S,2S)-2-(3-chloro-1- methyl-1H-indazol-6- yl)cyclopropane-1- carboxylate | 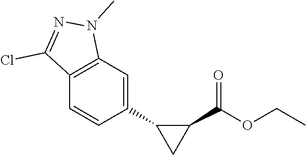 [SMILES: CCOC(=O)[C@H]1C[C@@H]1c1ccc2c(Cl)nn(C)c2c1] | 279.2 [M + H] |
| 397ª | ethyl (1S,2S)-2-(1,3- dimethyl-2-oxo-2,3-dihydro- 1H-benzo[d]imidazol-5- yl)cyclopropane-1- carboxylate | 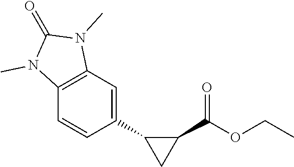 [SMILES: CCOC(=O)[C@H]1C[C@@H]1c1ccc2c(c1)n(C)c(=O)n2C] | 275.2 [M + H] |

^{a}Reaction time: 2.5 h

### Preparation 398

### (6-bromo-2-cyclopropylpyridin-3-yl)methyl methanesulfonate

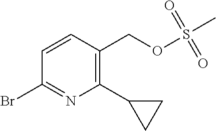 [SMILES: CS(=O)(=O)OCc1ccc(Br)nc1C1CC1]

(6-Bromo-2-cyclopropylpyridin-3-yl)methanol (1.00 g, 4.12 mmol), was dissolved in DCM (5 mL), and TEA (919 μL, 6.59 mmol) was added under N₂. The reaction mixture was cooled to 0° C., then methanesulfonyl chloride (383 μL, 4.95 mmol) was added dropwise over 5 min. After 10 min, water was poured (20 ml) into reaction and the mixture was allowed to warm to RT. The aqueous layer was extracted with DCM (3×) and the organic layers were combined, dried over sodium sulfate, and concentrated to obtain the title compound (1.26 g, 100%) as a yellow oil. ES-MS m/z 308.0 (M+H).

### Preparation 399

### 6-(bromomethyl)-1-methyl-1H-indazole

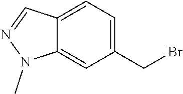

To 6-(Hydroxymethyl)-1-methylindazole (1.05 g, 6.46 mmol) was added PPh₃ (1.99 g, 7.57 mmol) and the mixture was purged with N₂. DCM (8 mL) was added, and the solution was cooled to 0° C. A solution of carbon tetrabromide (2.37 g, 7.15 mmol) in DCM (4 mL) was added dropwise. The reaction mixture was allowed to stir for 1 h, then was concentrated. The crude product was purified on silica, eluting with 0-100% EtOAc in hexane to obtain the title compound (1.24 g, 84%) as a white solid. ES-MS m/z 225.0, 227.0 (M+H).

### Preparation 400

### 1-((6-Bromo-2-cyclopropylpyridin-3-yl)methyl)tetrahydro-1H-thiophen-1-ium methanesulfonate

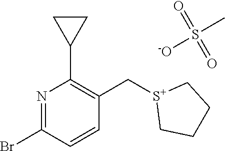

(6-Bromo-2-cyclopropylpyridin-3-yl)methyl methanesulfonate (1.26 g, 4.12 mmol, commercial reagent, Enamine: EN300-52434922) was dissolved in HFIP (10 mL). Tetrahydrothiophene (0.4 mL, 4.94 mmol) was added, and the mixture was stirred overnight. The reaction was partially concentrated, then MTBE was added, causing the desired product to crash out. The product was filtered and rinsed with MTBE, then dried under reduced pressure to obtain the title compound (1.82 g, 100%, 70% purity) as a clear oil. ES-MS m/z 298.0, 300.0 (M+).

### Preparation 401

### 1-((1-methyl-1H-indazol-6-yl)methyl)tetrahydro-1H-thiophen-1-ium bromide

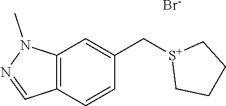 [SMILES: [Br-] ; Cn1ncc2ccc(C[S+]3CCCC3)cc21]

6-(bromomethyl)-1-methyl-1H-indazole (1.24 g, 5.4 mmol) was added to HFIP (21.0 mL), then tetrahydrothiophene (0.5 mL, 6 mmol) was added, and the solution was stirred at RT for 2 h. The reaction mixture was concentrated under reduced pressure, then diluted with MTBE (30 mL) causing the desired product to crash out. The product was filtered, washed with MTBE (10 mL×3) and dried under house vacuum. The title compound (1.68 g, 99%) was obtained as a white powder. ES-MS m/z 233.2 (M-Br).

### Preparation 402

### rac-(trans)-1-(6-bromo-2-cyclopropylpyridin-3-yl)-5-oxaspiro[2.4]heptan-4-one

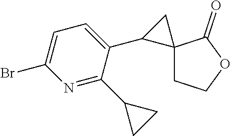 [SMILES: O=C1OCCC12CC2c1ccc(Br)nc1C1CC1]

1-((6-Bromo-2-cyclopropylpyridin-3-yl)methyl)tetrahydro-1H-thiophen-1-ium methanesulfonate (1.82 g, 70% Wt, 3.23 mmol) was dissolved in DCM (50 mL), then 3-methylenedihydrofuran-2(3H)-one (444 mg, 1.4 Eq, 4.52 mmol) was added, and the mixture was cooled to 0° C. Lithium bis(trimethylsilyl)amide (1.25 M in THF, 3.10 mL, 3.88 mmol) was then added dropwise and the reaction was allowed to warm to RT overnight. The mixture was quenched with sat. aq. ammonium chloride, and the aqueous layer was extracted with DCM (3×). The organic layers were dried over Na₂SO₄ and concentrated. The crude product was purified on silica, eluting with 0-100% 3:1 EtOAc:EtOH in heptane to afford the title compound (323 mg, 32% yield) as a yellow oil. ES-MS m/z 308.2 (M+H).

### Preparation 403

### rac-(trans)-1-(1-methyl-1H-indazol-6-yl)-5-oxaspiro[2.4]heptan-4-one

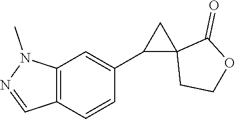 [SMILES: Cn1ncc2ccc(C3CC34CCOC4=O)cc21]

The title compound was prepared as described in Preparation 402 from 1-((1-methyl-1H-indazol-6-yl)methyl)tetrahydro-1H-thiophen-1-ium bromide (1.68 g, 5.37 mmol). ES-MS m/z 243.2 (M+H).

### Preparation 404

### (1R,3R)-1-(6-Bromo-2-methylpyridin-3-yl)-5-oxaspiro[2.4]heptan-4-one

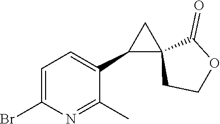 [SMILES: Cc1nc(Br)ccc1[C@H]1C[C@@]12CCOC2=O]

Rac-(trans)-1-(6-bromo-2-methylpyridin-3-yl)-5-oxaspiro[2.4]heptan-4-one (16.5 g, 98% Wt, 1 Eq, 57.3 mmol) was separated by chiral SFC purification (Chiralpak IG (250 mm×50 mm, 10 μm, 210 mL/min), eluted with 25% MeOH (with 0.1% NH₃H₂O) to afford the title compound (7.71 g, 46.5%) as the first eluting enantiomer. ES-MS m/z 281.7 (M+H).

### Preparation 405

### (R)-2-((tert-butoxycarbonyl)amino)-3-(1-(methyl-d3)-1H-pyrazol-4-yl)propanoic acid

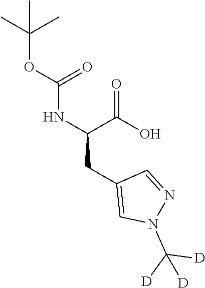 [SMILES: [2H]C([2H])([2H])n1cc(C[C@@H](NC(=O)OC(C)(C)C)C(=O)O)cn1]

Methyl (R)-2-((tert-butoxycarbonyl)amino)-3-(1-(methyl-d3)-1H-pyrazol-4-yl)propanoate (14.4 g, 50.3 mmol) was dissolved in THF (60 mL) under N₂ and water (30 mL) was added. Lithium hydroxide (3.61 g, 151 mmol) was added and the reaction was stirred overnight. The mixture was acidified to pH 3 using 1 M aq HCl (126 mL, 126 mmol) and the aqueous layer was extracted with EtOAc (3×300 mL). The combined organic extracts were dried over MgSO₄ and concentrated under reduced pressure to afford (R)-2-((tert-butoxycarbonyl)amino)-3-(1-(methyl-d3)-1H-pyrazol-4-yl)propanoic acid (13.4 g, 98%) as a semi-sticky white solid. (R)-2-((tert-butoxycarbonyl)amino)-3-(1-(methyl-d3)-1H-pyrazol-4-yl)propanoic acid (12.5 g, 45.8 mmol) was then dissolved in methyl acetate (374.1 mL) under N₂ and heated to 50° C. R-(+)-alpha-Phenylethylamine (6.43 mL, 50.4 mmol) was added, and the reaction mixture was allowed to cool to ambient temperature over the 2 days. The mixture was vacuum filtered and the filter cake was washed with methyl acetate (200 mL×2) before drying under vacuum to afford (R)-1-phenylethan-1-aminium (R)-2-((tert-butoxycarbonyl)amino)-3-(1-(methyl-d3)-1H-pyrazol-4-yl)propanoate (17.18 g, 95%) as a bright white solid. (R)-1-phenylethan-1-aminium (R)-2-((tert-butoxycarbonyl)amino)-3-(1-(methyl-d3)-1H-pyrazol-4-yl)propanoate (10.03 g, 25.49 mmol) was suspended in water (90 mL) and EtOAc (90 mL) was added. 12 M HCl (2.2 mL, 26 mmol) was added dropwise. Upon complete addition, the organic layer was collected, and the aqueous layer was extracted with EtOAc (90 mL×2). The combined organics were washed with sat. aq. NaCl (100 mL), dried over Na₂SO₄, and concentrated under reduced pressure to afford the title compound (7.83 g, 100%, 88.8% Purity) as a colorless foam. ES-MS m/z 273.2 (M+H).

### Preparation 406

### Methyl (R)-2-amino-3-(1-(methyl-d3)-1H-pyrazol-4-yl)propanoate hydrochloride

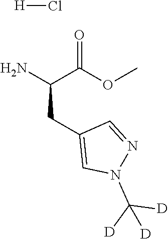

(R)-2-((tert-butoxycarbonyl)amino)-3-(1-(methyl-d3)-1H-pyrazol-4-yl)propanoic acid (7.83 g, 25.5 mmol, 88.8% purity) was dissolved in MeOH (35 mL), then 4 M HCl in 1,4-dioxane (35 mL, 0.14 mol) was added. The reaction mixture was stirred at RT overnight, then concentrated under a stream of Ar. The residue was further dried under vacuum to provide the title compound (5.69 g, 100%) as a colorless oil. ES-MS m/z 187.2 [M+H].

### Preparation 407

### tert-butyl (1-(5-(1-cyclopropyl-2-oxo-1,2-dihydropyrimidin-4-yl)-3-(trifluoromethyl)-1H-pyrazol-1-yl)-2-methylpropan-2-yl)carbamate

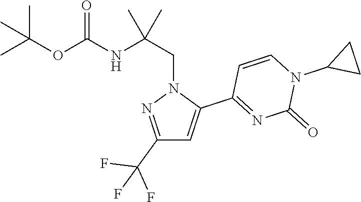 [SMILES: CC(C)(Cn1nc(C(F)(F)F)cc1-c1ccn(C2CC2)c(=O)n1)NC(=O)OC(C)(C)C]

To a solution of 4-chloro-1-cyclopropylpyrimidin-2(1H)-one (452 mg, 80% purity, 2.12 mmol) in 1,4-dioxane (20 mL) were added tert-butyl (2-methyl-1-(5-(4,4,5,5-tetramethyl-1,3,2-dioxaborolan-2-yl)-3-(trifluoromethyl)-1H-pyrazol-1-yl)propan-2-yl)carbamate (1.15 g, 2.52 mmol), Na₂CO₃ (443 mg, 4.18 mmol), bis[tris (tert-butyl)phosphine]palladium (226 mg, 442 μmol), and water (2 mL) at 20° C. The reaction mixture was degassed and refilled with N₂ (3×), then stirred at 60° C. for 0.5 h. Na₂SO₄ (˜5 g) was then added, and the resulting suspension was filtered through a short pad of silica gel, washing with EtOAc (100 mL). The filtrate was concentrated under reduced pressure to give a residue which was purified on silica, eluting with 0˜90% EtOAc in hexanes. The elution was concentrated under reduced pressure to give the title compound (373 mg, 38%) as light-yellow solid ES-MS m/z 442.1 (M+H)

### Preparation 408

### tert-Butyl (1-(5-(2-(2-hydroxypropan-2-yl)pyrimidin-5-yl)-3-(trifluoromethyl)-1H-pyrazol-1-yl)-2-methylpropan-2-yl)carbamate

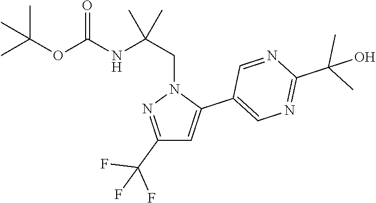 [SMILES: CC(C)(Cn1nc(C(F)(F)F)cc1-c1cnc(C(C)(C)O)nc1)NC(=O)OC(C)(C)C]

To tert-butyl (1-(5-(1,3,6,2-dioxazaborocan-2-yl)-3-(trifluoromethyl)-1H-pyrazol-1-yl)-2-methylpropan-2-yl)carbamate (24.00 g, 56.54 mmol, 99% purity), was added 2-(5-bromopyrimidin-2-yl)propan-2-ol (14.31 g, 62.63 mmol, 95% purity), 1 N Na₂CO₃ (aq.) (170 mL, 170 mmol), BrettPhos Pd G3 (5.21 g, 5.69 mmol, 99% purity), 1,4-dioxane (400 mL) and IPA (80 mL). The resulting mixture was evacuated and backfilled with N₂ (3×), stirred at 70° C. for 16 h, then filtered, rinsing with EtOAc (30 mL×3). The filtrate was concentrated under reduced pressure, then diluted with water (300 mL) and extracted with EtOAc (3 L×3). The combined organic extracts were dried over Na₂SO₄, filtered, and concentrated under reduced pressure. The crude product was purified on silica, eluting with 0-20% EtOAc/hexanes to provide the title compound (19.6 g, 74%) as a yellow solid. ES-MS m z=444.3 [M+H].

### Preparation 409

### tert-butyl (1-(5-(6-(2-hydroxypropan-2-yl)pyridin-3-yl)-3-(trifluoromethyl)-1H-pyrazol-1-yl)-2-methylpropan-2-yl)carbamate

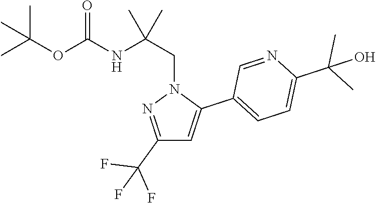 [SMILES: CC(C)(Cn1nc(C(F)(F)F)cc1-c1ccc(C(C)(C)O)nc1)NC(=O)OC(C)(C)C]

To 2-(5-bromopyridin-2-yl)propan-2-ol (2500 mg, 11.57 mmol) mixed with tert-butyl (1-(5-(1,3,6,2-dioxazaborocan-2-yl)-3-(trifluoromethyl)-1H-pyrazol-1-yl)-2-methylpropan-2-yl)carbamate (5.10 g, 12.1 mmol) was added BrettPhos-Pd-G3 (535 mg, 590 μmol), K₃PO₄ (6.140 g, 28.92 mmol), 1,4-dioxane (33.06 mL), water (6.611 mL), and IPA (6.611 mL). The reaction was purged with N₂ for 10 min, then stirred at 65° C. for 16 h. The mixture was cooled to RT, diluted with EtOAc, filtered through a short pad of celite and concentrated under reduced pressure. The resulting residue was redissolved in EtOAc, washed with water, sat. aq. NaCl, dried over Na₂SO₄, and concentrated. The crude product was purified on silica, eluting with 0-60% EtOAc/hexanes to afford the title compound (3.42 g, 66.8%) as a tan solid ES-MS m z 443.2 (M+H).

The compounds in the following table were prepared as described in Preparation 409 using the appropriate aryl bromide. Reactants can be added in different orders or in differing equivalency and differing methods can be used to work up or purify the compounds (normal phase, or high, neutral or low pH reversed phase). Such variances would be apparent to one skilled in the art.

TABLE 44

| | | | ES-MS |
|---|---|---|---|
| Preparation | Chemical Name | Structure | m/z |
| 410ª | tert-butyl (1-(5-(2-(2- hydroxypropan-2-yl)pyridin- 4-yl)-3-(trifluoromethyl)-1H- pyrazol-1-yl)-2- methylpropan-2-yl)carbamate | 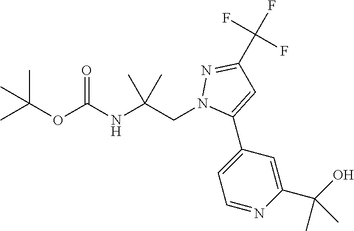 [SMILES: CC(C)(Cn1nc(C(F)(F)F)cc1-c1ccnc(C(C)(C)O)c1)NC(=O)OC(C)(C)C] | 443.2 (M + H) |
| 411^{b} | tert-butyl (2-methyl-1-(5-(6- methylpyridin-3-yl)-3- (trifluoromethyl)-1H-pyrazol- 1-yl)propan-2-yl)carbamate | 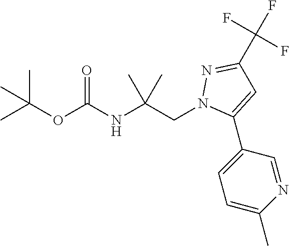 [SMILES: Cc1ccc(-c2cc(C(F)(F)F)nn2CC(C)(C)NC(=O)OC(C)(C)C)cn1] | 399.2 (M + H) |
| 412^{c} | tert-butyl (2-methyl-1-(5-(6- methylpyrazin-2-yl)-3- (trifluoromethyl)-1H-pyrazol- 1-yl)propan-2-yl)carbamate | 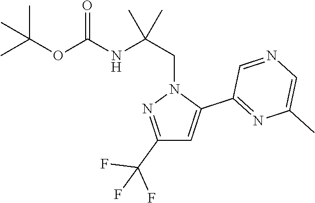 [SMILES: Cc1cncc(-c2cc(C(F)(F)F)nn2CC(C)(C)NC(=O)OC(C)(C)C)n1] | 300.1 (M + H) |
| 413^{d} | tert-butyl (1-(5-(3-methoxy-6- methylpyridazin-4-yl)-3- (trifluoromethyl)-1H-pyrazol- 1-yl)-2-methylpropan-2- yl)carbamate | 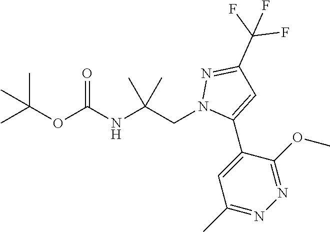 [SMILES: COc1nnc(C)cc1-c1cc(C(F)(F)F)nn1CC(C)(C)NC(=O)OC(C)(C)C] | 430.3 (M + H) |
| 414e | tert-butyl (1-(5-(4- (dimethylphosphoryl)phenyl)- 3-(trifluoromethyl)-1H- pyrazol-1-yl)-2- methylpropan-2-yl)carbamate | 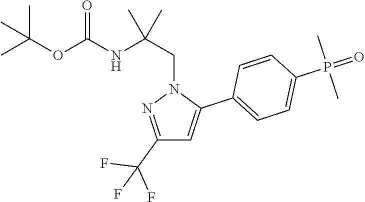 [SMILES: CC(C)(Cn1nc(C(F)(F)F)cc1-c1ccc(P(C)(C)=O)cc1)NC(=O)OC(C)(C)C] | 460.1 (M + H) |

aPurification conditions: Gradient 0-100% EtOAc in heptane.
^{b}Purification conditions: heptane and EtOAc (0-80%).
^{c}Purification conditions: 30% EtOAc in Cyclohexane
^{d}Pd(dppf)Cl₂•CH₂Cl₂ was used in place of BrettPhos Pd G3, and Cs₂CO₃ was used instead of K₃PO₄. No IPA was used. Reaction was stirred at 100° C. for 2.5 h.
^{e}XPhos Pd G3 was used in place of BrettPhos Pd G3 and Na₂CO₃ was used instead of K₃PO₄. No IPA was used. Reaction was stirred at 90° C. for 1 h.

### Preparation 415

### 2-(5-(1-(2-Amino-2-methylpropyl)-3-(trifluoromethyl)-1H-pyrazol-5-yl)pyrimidin-2-yl)propan-2-ol hydrochloride

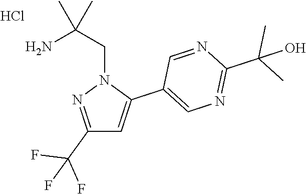 [SMILES: Cl ; CC(C)(N)Cn1nc(C(F)(F)F)cc1-c1cnc(C(C)(C)O)nc1]

To tert-butyl (1-(5-(2-(2-hydroxypropan-2-yl)pyrimidin-5-yl)-3-(trifluoromethyl)-1H-pyrazol-1-yl)-2-methylpropan-2-yl)carbamate (19.58 g, 41.94 mmol) in 1,4-dioxane (100 mL) was added 2 M HCl in 1,4-dioxane (210 mL, 420 mmol). The reaction mixture was stirred at 25° C. for 5 h, then filtered and washed with DCM (100 mL×3). The filter cake was dried under reduced pressure to afford the title compound (17.6 g, 43 mmol) as a white solid. ES-MS m/z 344.2 [M+H].

### Preparation 416

### 2-(5-(1-(2-amino-2-methylpropyl)-3-(trifluoromethyl)-1H-pyrazol-5-yl)pyridin-2-yl)propan-2-ol hydrochloride

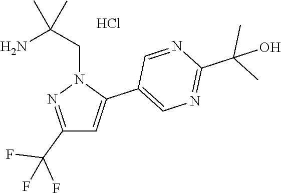 [SMILES: CC(C)(N)Cn1nc(C(F)(F)F)cc1-c1cnc(C(C)(C)O)nc1 ; Cl]

Tert-butyl (1-(5-(6-(2-hydroxypropan-2-yl)pyridin-3-yl)-3-(trifluoromethyl)-1H-pyrazol-1-yl)-2-methylpropan-2-yl)carbamate (3.4 g, 7.7 mmol) was dissolved in EtOH (39 mL) and 4 M HCl in 1,4-dioxane (38.7 mL, 154.6 mmol) was added. The mixture was stirred at RT for 3 h then concentrated under reduced pressure. EtOH (50 mL) was added, and the mixture was concentrated under reduced pressure (repeated 4×) resulting in a solid which was vacuum dried to give the title compound (2.56 g, 87.4%) as a tan solid. ES-MS m/z 343.0 (M+H)

The compounds in the following table were prepared as described in Preparation 416 using the appropriate amine and carboxylic acid. Reactants can be added in different orders or in differing equivalency and differing methods can be used to work up or purify the compounds (normal phase, or high, neutral or low pH reversed phase). Such variances would be apparent to one skilled in the art.

TABLE 45

| | | | ES-MS |
|---|---|---|---|
| Preparation | Chemical Name | Structure | m/z |
| 417ª | 2-(4-(1-(2-amino-2- methylpropyl)-3- (trifluoromethyl)-1H- pyrazol-5-yl)pyridin-2- yl)propan-2-ol dihydrochloride | 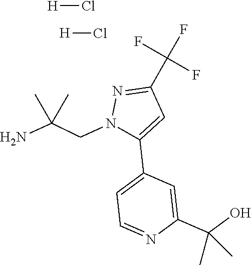 | 443.2 (M + H) |
| 418^{b} | 2-methyl-1-(5-(6- methylpyridin-3-yl)-3- (trifluoromethyl)-1H- pyrazol-1-yl)propan-2- amine hydrochloride | 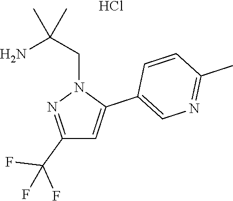 [SMILES: Cl ; Cc1ccc(-c2cc(C(F)(F)F)nn2CC(C)(C)N)cn1] | 299.2 (M + H) |
| 419ª | 2-methyl-1-(5-(6- methylpyrazin-2-yl)-3- (trifluoromethyl)-1H- pyrazol-1-yl)propan-2- amine hydrochloride | 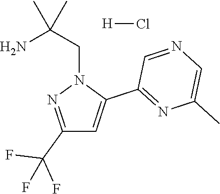 | 300.1 (M + H) |
| 420^{b} | 4-(1-(2-amino-2- methylpropyl)-3- (trifluoromethyl)-1H- pyrazol-5-yl)-1- cyclopropylpyrimidin- 2(1H)-one hydrochloride | 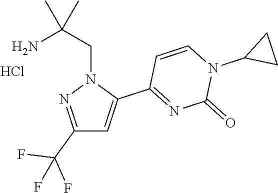 [SMILES: Cl ; CC(C)(N)Cn1nc(C(F)(F)F)cc1-c1ccn(C2CC2)c(=O)n1] | 342.1 (M + H) |
| 421^{c} | (4-(1-(2-amino-2- methylpropyl)-3- (trifluoromethyl)-1H- pyrazol-5- yl)phenyl)dimethylphosphine oxide hydrochloride | 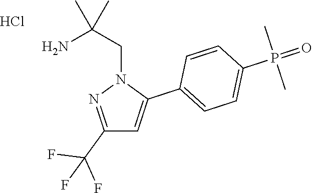 [SMILES: Cl ; CC(C)(N)Cn1nc(C(F)(F)F)cc1-c1ccc(P(C)(C)=O)cc1] | 360.2 (M + H) |
| 422^{c} | (1-(2-amino-2- methylpropyl)-3- (trifluoromethyl)-1H- pyrazol-5- yl)dimethylphosphine oxide hydrochloride | 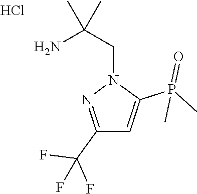 [SMILES: Cl ; CC(C)(N)Cn1nc(C(F)(F)F)cc1P(C)(C)=O] | 284.2 (M + H) |

^{a}compound obtained as a thick yellow oil
^{b}obtained as a white powder
^{c}Reaction was run in 1,4-dioxane instead of EtOH.

### Preparation 423

### Methyl (R)-2-((1R,2R)-2-(6-chloro-2-methylpyridin-3-yl)-1-(2-chloroethyl)cyclopropane-1-carboxamido)-3-(1-(difluoromethyl)-1H-pyrazol-4-yl)propanoate

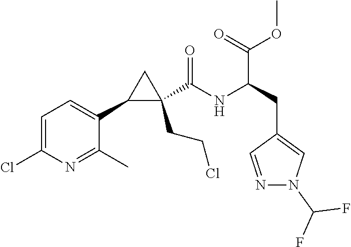 [SMILES: COC(=O)[C@@H](Cc1cnn(C(F)F)c1)NC(=O)[C@@]1(CCCl)C[C@@H]1c1ccc(Cl)nc1C]

To a mixture of (1R,3R)-1-(6-bromo-2-methylpyridin-3-yl)-5-oxaspiro[2.4]heptan-4-one (3.89 g, 13.8 mmol) and benzyltriethylammonium chloride (4.8 g, 21 mmol) was added DCE (39 mL), SOCl₂ (3.0 mL, 41 mmol) and BF₃·Et₂₀O (2.6 mL, 21 mmol). The reaction mixture was heated to 80° C. overnight, then cooled to RT and concentrated under reduced pressure to obtain a quantitative yield of (1R,2R)-2-(6-chloro-2-methylpyridin-3-yl)-1-(2-chloroethyl)cyclopropane-1-carbonyl chloride. In a separate vessel, a mixture of methyl (R)-2-amino-3-(1-(difluoromethyl)-1H-pyrazol-4-yl)propanoate (4.03 g, 13.8 mmol) and TEA (18 mL, 0.13 mol) in DCM (126 mL) was stirred at 0° C. A suspension of (1R,2R)-2-(6-chloro-2-methylpyridin-3-yl)-1-(2-chloroethyl)cyclopropane-1-carbonyl chloride (6.3 g, 25 mmol) in DCM (100 mL) was added dropwise. The reaction was stirred at 0° C. for 30 min, then quenched with sat. aq. ammonium chloride (100 mL) and diluted with water (100 mL). The mixture was stirred at 0° C. for 90 min. The organic layer was collected and the aqueous layer extracted with additional DCM (100 mL×2). The combined organic extracts were concentrated at 25° C. under reduced pressure to obtain the title compound (19.1 g, 100%, 35% purity) as an orange oil. ES-MS m/z 475.2 (M+H).

### Preparation 424

### Methyl (R)-2-((1R,2R)-2-(6-chloro-2-methylpyridin-3-yl)-1-(2-chloroethyl)cyclopropane-1-carboxamido)-3-(1-(methyl-d3)-1H-pyrazol-4-yl)propanoate

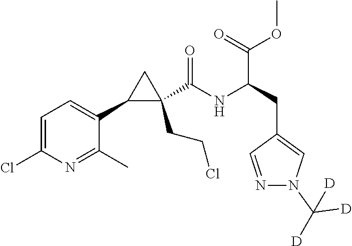 [SMILES: [2H]C([2H])([2H])n1cc(C[C@@H](NC(=O)[C@@]2(CCCl)C[C@@H]2c2ccc(Cl)nc2C)C(=O)OC)cn1]

The title compound was prepared as described in Preparation 423 from (1R,3R)-1-(6-bromo-2-methylpyridin-3-yl)-5-oxaspiro[2.4]heptan-4-one (3.13 g, 11.1 mmol) and methyl (R)-2-amino-3-(1-(methyl-d3)-1H-pyrazol-4-yl)propanoate hydrochloride (2.85 g, 12.8 mmol). ES-MS m/z 442.2 (M+H).

### Preparation 425

### (trans)-1-(2-chloroethyl)-N—((R)-3-(1-(difluoromethyl)-1H-pyrazol-4-yl)-1-((1-(3-(5-fluoro-1-methyl-6-oxo-1,6-dihydropyridin-3-yl)-5-(trifluoromethyl)-1H-pyrazol-1-yl)-2-methylpropan-2-yl)amino)-1-oxopropan-2-yl)-2-(1-methyl-1H-indazol-6-yl)cyclopropane-1-carboxamide

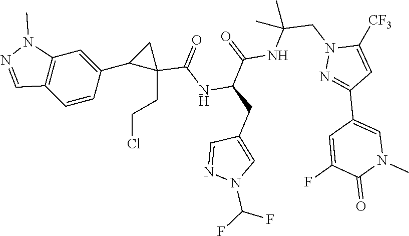 [SMILES: Cn1cc(-c2cc(C(F)(F)F)n(CC(C)(C)NC(=O)[C@@H](Cc3cnn(C(F)F)c3)NC(=O)C3(CCCl)CC3c3ccc4cnn(C)c4c3)n2)cc(F)c1=O]

The title compound was prepared as described in Preparation 423 from rac-(trans)-1-(1-methyl-1H-indazol-6-yl)-5-oxaspiro[2.4]heptan-4-o (403.7 mg, 1.67 mmol) and (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (454.4 mg, 874.8 μmol). ES-MS m/z 780.4 (M+H).

### Preparation 426

### (1R,2R)-2-(6-chloro-2-methylpyridin-3-yl)-1-(2-chloroethyl)-N—((R)-3-(1-(difluoromethyl)-1H-pyrazol-4-yl)-1-((1-(3-(5-fluoro-1-methyl-6-oxo-1,6-dihydropyridin-3-yl)-5-(trifluoromethyl)-1H-pyrazol-1-yl)-2-methylpropan-2-yl)amino)-1-oxopropan-2-yl)cyclopropane-1-carboxamide

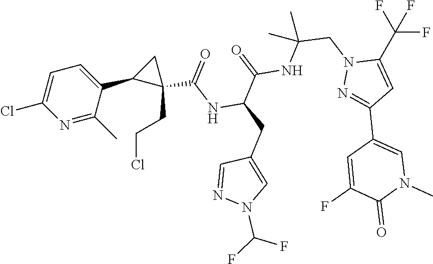 [SMILES: Cc1nc(Cl)ccc1[C@H]1C[C@]1(CCCl)C(=O)N[C@H](Cc1cnn(C(F)F)c1)C(=O)NC(C)(C)Cn1nc(-c2cc(F)c(=O)n(C)c2)cc1C(F)(F)F]

Step 1—To a mixture of (1R,3R)-1-(6-bromo-2-methylpyridin-3-yl)-5-oxaspiro[2.4]heptan-4-one (1.02 g, 3.62 mmol) and benzyltriethylammonium chloride (1.25 g, 5.49 mmol) was added DCE (15.0 mL). The vial was sealed and purged with N₂, then treated with SOCl₂ (800 μL, 11.0 mmol) dropwise at 0° C. followed by BF₃·Et₂O (700 μL, 5.67 mmol). The reaction mixture was heated to 80° C. overnight, then cooled to RT and concentrated under reduced pressure to obtain a quantitative yield of (1R,2R)-2-(6-chloro-2-methylpyridin-3-yl)-1-(2-chloroethyl)cyclopropane-1-carbonyl chloride as a dark gel.

Step 2—In a separate vessel, a mixture of of (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (1.89 g, 3.62 mmol) and TEA (3.5 mL, 25 mmol) in DCM (6.0 mL) was stirred at 0° C. A suspension of (1R,2R)-2-(6-chloro-2-methylpyridin-3-yl)-1-(2-chloroethyl)cyclopropane-1-carbonyl chloride (1.06 g, 3.63 mmol) in DCM (6.0 mL) was added dropwise. The cooling bath was removed, and the reaction mixture was stirred at RT for 0.75 h. The crude product was purified on silica, eluting with 0-60% EtOAc:EtOH (3:1) in heptane to obtain the title compound (2.66 g, 74%) as a pale-brown solid. ES-MS m/z 775.4 (M+H).

### Preparation 426a

### (1R,2R)-2-(6-chloro-2-methylpyridin-3-yl)-1-(2-chloroethyl)-N—((R)-3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)cyclopropane-1-carboxamide

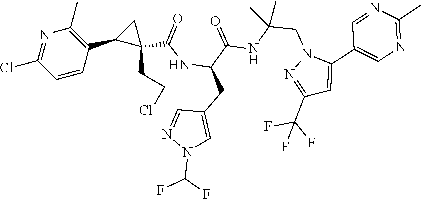 [SMILES: Cc1ncc(-c2cc(C(F)(F)F)nn2CC(C)(C)NC(=O)[C@@H](Cc2cnn(C(F)F)c2)NC(=O)[C@@]2(CCCl)C[C@@H]2c2ccc(Cl)nc2C)cn1]

The title compound was prepared as described in Preparation 426 using (1R,3R)-1-(6-bromo-2-methylpyridin-3-yl)-5-oxaspiro[2.4]heptan-4-one and (R)-2-amino-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide hydrochloride, using THE as the reaction solvent in Step 2. The product was purified by silica gel chromatography using a gradient of 2% MeOH in DCM. ES-MS m/z 742 (M+H).

### Preparation 427

### (trans)-2-(6-Bromo-2-cyclopropylpyridin-3-yl)-1-(2-chloroethyl)-N—((R)-1-((1-(5-(1-cyclopropyl-2-oxo-1,2-dihydropyridin-4-yl)-3-(trifluoromethyl)-1H-pyrazol-1-yl)-2-methylpropan-2-yl)amino)-3-(1-(difluoromethyl)-1H-pyrazol-4-yl)-1-oxopropan-2-yl)cyclopropane-1-carboxamide (mixture of isomers)

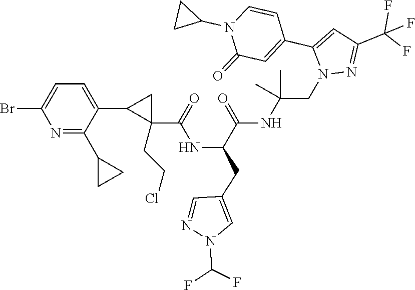 [SMILES: CC(C)(Cn1nc(C(F)(F)F)cc1-c1ccn(C2CC2)c(=O)c1)NC(=O)[C@@H](Cc1cnn(C(F)F)c1)NC(=O)C1(CCCl)CC1c1ccc(Br)nc1C1CC1]

To a suspension of rac-(trans)-1-(6-bromo-2-cyclopropylpyridin-3-yl)-5-oxaspiro[2.4]heptan-4-one (323 mg, 1.05 mmol) in DCE (16 mL) was added boron trifluoride etherate (265 μL, 2.11 mmol), benzyl (triethyl) ammonium chloride (480 mg, 2.11 mmol), and thionyl chloride (309 μL, 4.20 mmol) at 0° C. The resulting suspension was heated to 80° C. for overnight. The reaction mixture was concentrated under vacuum to obtain rac-(trans)-2-(6-bromo-2-cyclopropylpyridin-3-yl)-1-(2-chloroethyl)cyclopropane-1-carbonyl chloride. To a suspension of diisopropylethylamine (1.20 mL, 6.96 mmol) and (R)-2-amino-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide hydrochloride (712 mg, 1.26 mmol) in DCM (16 mL) at 0° C. was added rac-(trans)-2-(6-bromo-2-cyclopropylpyridin-3-yl)-1-(2-chloroethyl)cyclopropane-1-carbonyl chloride dissolved in DCM (2 mL). The reaction was allowed to stir for 30 min, then was loaded onto celite and purified on silica, eluting with 0-100% 3:1 EtOAc:EtOH in heptane. The title compound was isolated as a mixture of the 2-bromo and 2-chloropyridyl analogues (895 mg, 100%) as an orange oil. ES-MS m/z 855.2 (M+H).

### Preparation 428

### methyl (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate

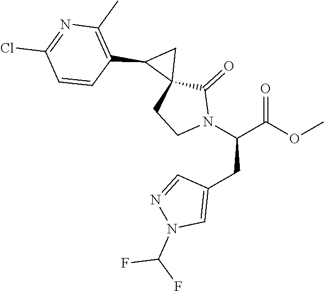 [SMILES: COC(=O)[C@@H](Cc1cnn(C(F)F)c1)N1CC[C@@]2(C[C@@H]2c2ccc(Cl)nc2C)C1=O]

Methyl (R)-2-((1R,2R)-2-(6-chloro-2-methylpyridin-3-yl)-1-(2-chloroethyl)cyclopropane-1-carboxamido)-3-(1-(difluoromethyl)-1H-pyrazol-4-yl)propanoate (19.1 g, 35% purity, 14.1 mmol) was co-evaporated with 50 mL ACN to remove residual solvents. The resulting oil was dissolved in ACN (80 mL) and Cs₂CO₃ (11.4 g, 35.0 mmol) was added. The suspension was stirred at RT for 4 h, stored at −20° C. overnight, then concentrated under reduced pressure. The residue was quenched with sat. aq. NH₄Cl (100 mL) and extracted with DCM (100 mL×4). The combined extracts were concentrated at 25° C. under reduced pressure. Flash chromatography (SiO₂, gradient 30-80% EtOAc in heptane) provided the title compound (4.05 g, 62%) as an orange oil. ES-MS m/z 439.2/441.2 (M+H).

### Preparation 429

### methyl (R)-2-((1R,3R)-1-(6-chloro-2-methylpyridin-3-yl)-4-oxo-5-azaspiro[2.4]heptan-5-yl)-3-(1-(methyl-d3)-1H-pyrazol-4-yl)propanoate

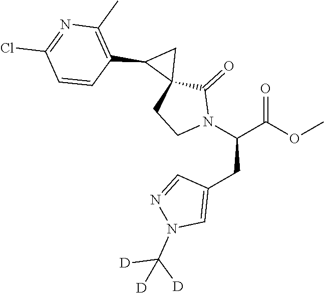 [SMILES: [2H]C([2H])([2H])n1cc(C[C@H](C(=O)OC)N2CC[C@@]3(C[C@@H]3c3ccc(Cl)nc3C)C2=O)cn1]

Methyl (R)-2-((1R,2R)-2-(6-chloro-2-methylpyridin-3-yl)-1-(2-chloroethyl)cyclopropane-1-carboxamido)-3-(1-(methyl-d3)-1H-pyrazol-4-yl)propanoate (4.91 g, 11.1 mmol) was charged was dissolved in ACN (100 mL), and cesium carbonate (8.0 g, 25 mmol) was added. The reaction was allowed to stir for 4 days, then was concentrated at 25° C. under reduced pressure, diluted with H₂O (50 mL) and extracted with DCM (50 mL×4). The combined organics were concentrated at 35° C. under reduced pressure and purified on silica, eluting with 30-100% EtOAc in heptane followed by 0-10% MeOH in EtOAc.

The purified solid was redissolved in tBuOH (˜50 mL) at 50° C. and concentrated until the mixture became cloudy. The mixture was then diluted to 40 g total weight with H₂O and MeOH was added until homogeneous (˜30 mL). 1 M aq HCl (2.0 mL, 2.0 mmol) was then added, and the mixture was stirred at RT for 5 h. The reaction was quenched with 20% aq NH₄OH (˜0.5 mL) and concentrated under reduced pressure. The crude product was purified on silica, eluting with 0-100% (3:1 EtOAc:EtOH) in heptane to afford the title compound (20 g, 51%, 11.4% purity) as a solution in tBuOH. ES-MS m/z 406.2/408.2 (M+H).

### Preparation 430

### methyl (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(difluoromethyl)-1H-pyrazol-4-yl)propanoate

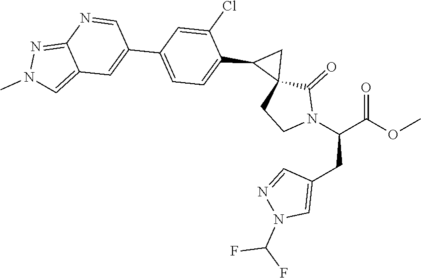

To a mixture of methyl (R)-2-((1R,3S)-1-(4-bromo-2-chlorophenyl)-4-oxo-5-azaspiro[2.4]heptan-5-yl)-3-(1-(difluoromethyl)-1H-pyrazol-4-yl)propanoate (85 mg, 0.16 mmol), was added (2-Methyl-2H-pyrazolo[3,4-b]pyridin-5-yl) boronic acid (29 mg, 0.16 mmol), Sodium carbonate (51 mg, 0.48 mmol) and XPhos Pd G3 (20 mg, 024 μmol) in 1,4-dioxane (3 mL) and H₂O (0.3 mL). The mixture was degassed and purged with N₂ (3×) at 20° C. then was heated up to 90° C. and stirred for 1 h. The reaction was cooled, then diluted with H₂O (10 mL) and extracted with EtOAc (20 mL). The organic layer was washed with sat. aq. NaCl (20 mL×2), dried over Na₂SO₄ and filtered. The filtrate was concentrated under reduced pressure to give a dark yellow oil which was purified on silica, eluting with 0˜4% MeOH in DCM to give the title compound (89 mg, 0.15 mmol, 93%) as a yellow oil. ES-MS m/z 555.1, 555.7 (M+H).

### Preparation 431

### (1S,2S)-2-(2-fluoro-4-hydroxy-5-((methoxycarbonyl)(methyl)amino)phenyl)cyclopropane-1-carboxylic acid

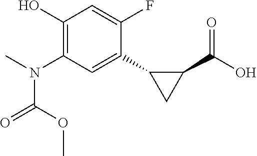 [SMILES: COC(=O)N(C)c1cc([C@H]2C[C@@H]2C(=O)O)c(F)cc1O]

To ethyl (1S,2S)-2-(6-fluoro-3-methyl-2-oxo-2,3-dihydrobenzo[d]oxazol-5-yl)cyclopropane-1-carboxylate (174 mg, 610 μmol) was added 2M aq. LiOH (4.0 mL, 8.0 mmol) in THF (3 mL) and MeOH (3 mL). The resulting mixture was stirred at RT for 2 h, quenched with 4M aq. HCl (1.8 mL, 7.2 mmol), then stirred for an additional 5 min. The reaction was concentrated under a stream of N₂ to obtain a quantitative yield of the title compound as a dark solid. ES-MS m/z 284.0 (M+H).

The compounds in the following table were prepared as described in Preparation 431 using the appropriate ethyl ester. Reactants can be added in different orders or in differing equivalency and differing methods can be used to work up or purify the compounds (normal phase, or high, neutral or low pH reversed phase). Such variances would be apparent to one skilled in the art. In the following preparations, ring opening was not observed.

TABLE 46

| | | | ES-MS |
|---|---|---|---|
| Preparation | Chemical Name | Structure | m/z |
| 432 | (1S,2S)-2-(3-chloro-1-methyl-1H- indazol-6-yl)cyclopropane-1- carboxylic acid | 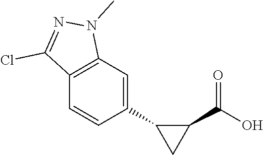 [SMILES: Cn1nc(Cl)c2ccc([C@H]3C[C@@H]3C(=O)O)cc21] | 251.2/253.2 (M + H) |
| 433 | (1S,2S)-2-(1,3-dimethyl-2-oxo- 2,3-dihydro-1H-benzo[d]imidazol- 5-yl)cyclopropane-1-carboxylic acid | 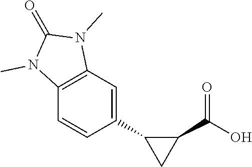 [SMILES: Cn1c(=O)n(C)c2cc([C@H]3C[C@@H]3C(=O)O)ccc21] | 247.2 (M + H) |

Preparation 434 Lithium (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate

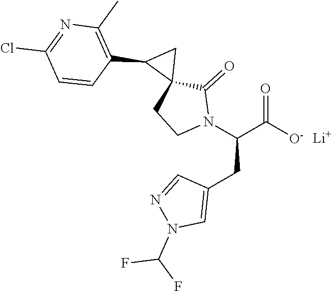 [SMILES: Cc1nc(Cl)ccc1[C@H]1C[C@@]12CCN([C@H](Cc1cnn(C(F)F)c1)C(=O)[O-])C2=O ; [Li+]]

Methyl (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate (4.8 g, 11 mmol) was co-evaporated with tBuOH (30 mL) to remove any residual solvents. The resulting oil was dissolved in tBuOH (50 mL) and diluted with H₂O (18 mL). The mixture was cooled to 0° C. and treated dropwise with 2 M aq. LiOH (7.0 mL, 14 mmol). The reaction was stirred at 0° C. for 1 h, quenched with 1 M aq. HCl (3.8 mL, 3.8 mmol), partially concentrated under reduced pressure, and lyophilized to provide the title compound (4.96 g, 100%, 95% purity) as an off-white solid. ES-MS m/z 425.2/427.2 (M+H).

### Preparation 435

### Lithium (R)-2-((1R,3R)-1-(6-chloro-2-methylpyridin-3-yl)-4-oxo-5-azaspiro[2.4]heptan-5-yl)-3-(1-(methyl-d3)-1H-pyrazol-4-yl)propanoate

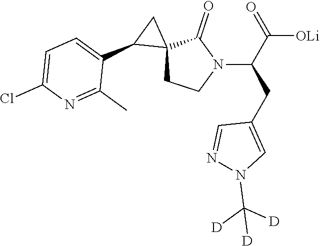

Title compound was prepared as described in Preparation 434 from methyl (R)-2-((1R,3R)-1-(6-chloro-2-methylpyridin-3-yl)-4-oxo-5-azaspiro[2.4]heptan-5-yl)-3-(1-(methyl-d3)-1H-pyrazol-4-yl)propanoate (20.7 g, 11.4% purity, 5.81 mmol). ES-MS m/z 392.2/394.2 (M+H) (C135/37).

### Preparation 436

### (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)propanoic acid

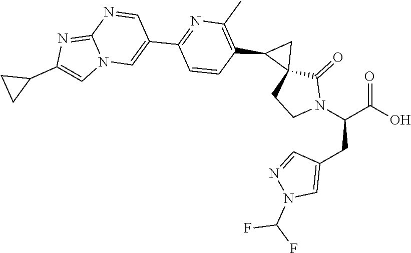 [SMILES: Cc1nc(-c2cnc3nc(C4CC4)cn3c2)ccc1[C@H]1C[C@@]12CCN([C@H](Cc1cnn(C(F)F)c1)C(=O)O)C2=O]

To lithium (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate (673 mg, 1.48 mmol), was added bis(di-tert-butyl (4-dimethylaminophenyl)phosphine)dichloropalladium (II) (62 mg, 88 μmol), (2-cyclopropylimidazo[1,2-a]pyrimidin-6-yl) boronic acid (422 mg, 2.08 mmol), Na₂CO₃ (400 mg, 3.77 mmol), and MeOH (8.0 mL). The headspace was flushed with Ar and the reaction mixture was sparged with Ar while stirring at RT. The reaction was stirred at 65° C. overnight, cooled to RT, treated with SiliaMetS thiol resin (Silicycle, 1.46 mmol/g, 1 g), stirred at RT for 30 min, diluted with 6 mL H₂O, and quenched with FA (500 μL, 13.3 mmol). Reverse-phase flash chromatography (C18, gradient 0-100% ACN in 0.1% aqueous FA) provided the title compound (589 mg, 69%) as a yellow powder. ES-MS m/z 548.2 (M+H).

### Preparation 437

### (R)-3-(1-(difluoromethyl)-1H-pyrazol-4-yl)-2-((1R,3R)-1-(6-(2-(methoxymethyl) imidazo[1,2-a]pyrimidin-6-yl)-2-methylpyridin-3-yl)-4-oxo-5-azaspiro[2.4]heptan-5-yl)propanoic acid

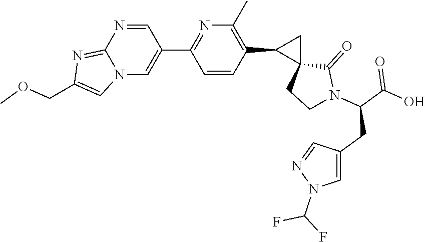 [SMILES: COCc1cn2cc(-c3ccc([C@H]4C[C@@]45CCN([C@H](Cc4cnn(C(F)F)c4)C(=O)O)C5=O)c(C)n3)cnc2n1]

Title compound was prepared as described in Preparation 436 from lithium (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate (309 mg, 681 μmol, 95% purity) and (2-(methoxymethyl) imidazo[1,2-a]pyrimidin-6-yl) boronic acid (144 mg, 696 μmol). ES-MS m/z 552.4 (M+H).

### Preparation 438

### (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(difluoromethyl)-1H-pyrazol-4-yl)propanoic acid

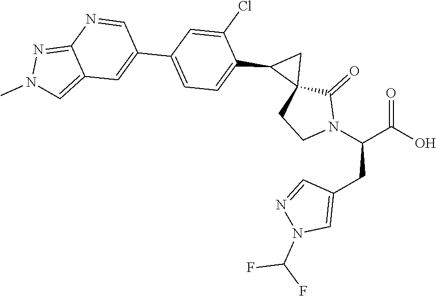 [SMILES: Cn1cc2cc(-c3ccc([C@H]4C[C@@]45CCN([C@H](Cc4cnn(C(F)F)c4)C(=O)O)C5=O)c(Cl)c3)cnc2n1]

To a mixture of methyl (R)-2-((1R,3S)-1-(2-chloro-4-(2-methyl-2H-pyrazolo[3,4-b]pyridin-5-yl)phenyl)-4-oxo-5-azaspiro[2.4]heptan-5-yl)-3-(1-(difluoromethyl)-1H-pyrazol-4-yl)propanoate (88 mg, 0.15 mmol) in tBuOH (3 mL) and H₂O (1.5 mL) was added lithium hydroxide monohydrate (20 mg, 0.48 mmol) at 0° C. The reaction mixture was stirred at 0° C. for 1 h, then diluted with H₂O (20 mL) and extracted with EtOAc (20 mL). 1 N aq. HCl was added until the aq layer pH=1, and the aqueous layer was extracted with EtOAc (20 mL). The resulting organic layer was washed with sat. aq. NaCl (30 mL×2), dried over Na₂SO₄, filtered, and concentrated under reduced pressure to give the title compound (71 mg, 84%) as a white solid. ES-MS m/z 541.1 (M+H).

### Preparation 439

### (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide

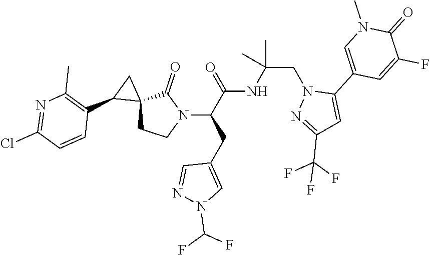 [SMILES: Cc1nc(Cl)ccc1[C@H]1C[C@@]12CCN([C@H](Cc1cnn(C(F)F)c1)C(=O)NC(C)(C)Cn1nc(C(F)(F)F)cc1-c1cc(F)c(=O)n(C)c1)C2=O]

A mixture of (1R,2R)-2-(6-chloro-2-methylpyridin-3-yl)-1-(2-chloroethyl)-N—((R)-3-(1-(difluoromethyl)-1H-pyrazol-4-yl)-1-((1-(3-(5-fluoro-1-methyl-6-oxo-1,6-dihydropyridin-3-yl)-5-(trifluoromethyl)-1H-pyrazol-1-yl)-2-methylpropan-2-yl)amino)-1-oxopropan-2-yl)cyclopropane-1-carboxamide (2.66 g, 78% Wt, 2.68 mmol) and Cs₂CO₃ (1.87 g, 5.74 mmol) in NMP (10 mL) was stirred for 65 h at RT. The reaction mixture was partitioned between H₂O (50 mL) and EtOAc (50 mL) and the aqueous layer was extracted with EtOAc (50 mL×2). The organic layers were combined, washed with sat. aq. NaCl (50 mL×3), dried over Na₂SO₄, and concentrated under reduced pressure. The residue was purified by on silica, eluting with (0-100%) EtOAc in hexane to obtain the title compound (2.81 g, 100%, 74% purity) as a yellow oil. ES-MS m/z 739.4/741.4 (M+H).

### Preparation 439a

### (R)-2-((1R,3R)-1-(6-Chloro-2-methylpyridin-3-yl)-4-oxo-5-azaspiro[2.4]heptan-5-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide

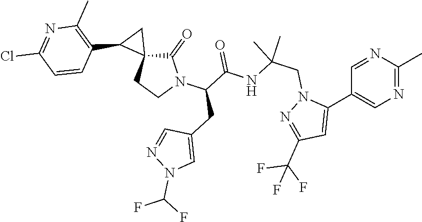 [SMILES: Cc1ncc(-c2cc(C(F)(F)F)nn2CC(C)(C)NC(=O)[C@@H](Cc2cnn(C(F)F)c2)N2CC[C@@]3(C[C@@H]3c3ccc(Cl)nc3C)C2=O)cn1]

The title compound was prepared as described in Preparation 439 using (1R,2R)-2-(6-chloro-2-methylpyridin-3-yl)-1-(2-chloroethyl)-N—((R)-3-(1-(difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)cyclopropane-1-carboxamide and using ACN as the reaction solvent. The product was purified by silica gel chromatography using isocratic 4% MeOH in DCM. ES-MS m/z 706 (M+H).

### Preparation 440

### (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)-N-(1-(5-(6-(2-hydroxypropan-2-yl)pyridin-3-yl)-3-(trifluoromethyl)-1H-pyrazol-1-yl)-2-methylpropan-2-yl)propanamide

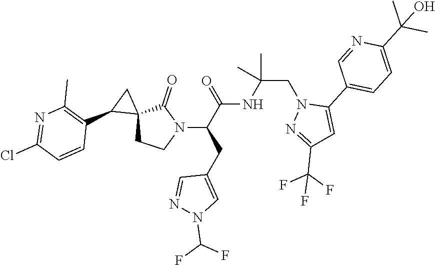 [SMILES: Cc1nc(Cl)ccc1[C@H]1C[C@@]12CCN([C@H](Cc1cnn(C(F)F)c1)C(=O)NC(C)(C)Cn1nc(C(F)(F)F)cc1-c1ccc(C(C)(C)O)nc1)C2=O]

Lithium (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate (420 mg, 975 μmol) was combined with 2-(5-(1-(2-amino-2-methylpropyl)-3-(trifluoromethyl)-1H-pyrazol-5-yl)pyridin-2-yl)propan-2-ol hydrochloride (450 mg, 1.19 mmol) and 4-methylmorpholine (500 μL, 4.55 mmol) in DMA (9.75 mL). The mixture was stirred at 0 C for 10 min, then DEPBT (586 mg, 1.96 mmol) was added and the reaction was allowed to warm to RT over 12 h. The reaction was diluted with EtOAc/water and the organic layer was separated, washed with sat. aq. NaCl and dried over Na₂SO₄. The solution was concentrated under reduced pressure to give a yellow oily residue that was purified on silica, eluting with 50% 3:1 EtOAc:EtOH in cyclohexane to give the title compound as a yellow oil (403 mg, 0.52 mmol, 54%, 97% Purity).

The compounds in the following table were prepared as described in Preparation 440 using the appropriate amine and carboxylic acid. Reactants can be added in different orders or in differing equivalency and differing methods can be used to work up or purify the compounds (normal phase, or high, neutral or low pH reversed phase). Such variances would be apparent to one skilled in the art.

TABLE 47

| | | | ES-MS |
|---|---|---|---|
| Preparation | Chemical Name | Structure | m/z |
| 441^{a} | (R)-2-((1R,3R)-1-(6- chloro-2- methylpyridin-3- yl)-4-oxo-5- azaspiro[2.4] heptan-5-yl)- 3-(1- (difluoromethyl)-1H- pyrazol-4-yl)-N-(2- methyl-1-(5-(6- methylpyrazin-2-yl)-3- (trifluoromethyl)-1H- pyrazol-1-yl)propan-2- yl)propanamide | 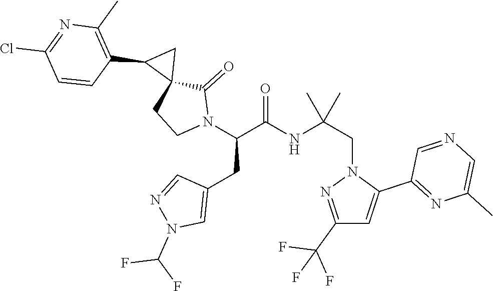 [SMILES: Cc1cncc(-c2cc(C(F)(F)F)nn2CC(C)(C)NC(=O)[C@@H](Cc2cnn(C(F)F)c2)N2CC[C@@]3(C[C@@H]3c3ccc(Cl)nc3C)C2=O)n1] | 706.3 (M + H) |
| 442 | (R)-2-((1R,3R)-1-(6- chloro-2- methylpyridin-3- yl)-4-oxo-5- azaspiro[2.4] heptan-5-yl)- N-(2-methyl-1-(5-(6- methylpyridin-3-yl)-3- (trifluoromethyl)-1H- pyrazol-1-yl) propan-2-yl)- 3-(1-(methyl-d3)-1H- pyrazol-4-yl) propanamide | 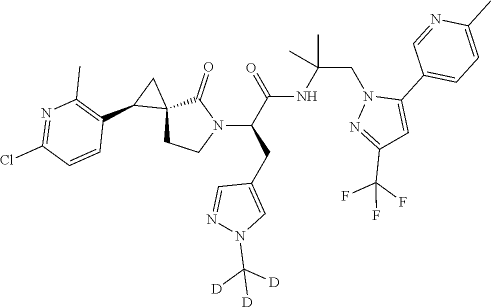 [SMILES: [2H]C([2H])([2H])n1cc(C[C@H](C(=O)NC(C)(C)Cn2nc(C(F)(F)F)cc2-c2ccc(C)nc2)N2CC[C@@]3(C[C@@H]3c3ccc(Cl)nc3C)C2=O)cn1] | 672.4 (M + H) |

^{a}Compound carried forward without purification

### Preparation 443

### (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

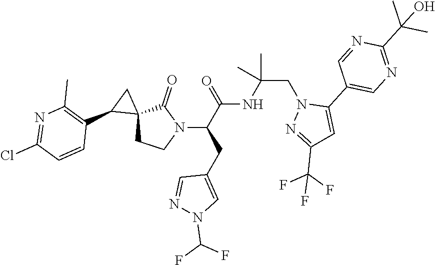 [SMILES: Cc1nc(Cl)ccc1[C@H]1C[C@@]12CCN([C@H](Cc1cnn(C(F)F)c1)C(=O)NC(C)(C)Cn1nc(C(F)(F)F)cc1-c1cnc(C(C)(C)O)nc1)C2=O]

To a mixture of 2-(5-(1-(2-amino-2-methylpropyl)-3-(trifluoromethyl)-1H-pyrazol-5-yl)pyrimidin-2-yl)propan-2-ol (259.7 mg, 711.0 μmol), lithium (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate (201.2 mg, 443.7 μmol) in DMA (4.0 mL), was added 4-methylmorpholine (0.35 mL, 3.2 mmol). The reaction mixture was cooled to 0° C. and Diethyl (4-oxo-1,2,3-benzotriazin-3-yl)phosphate (288.1 mg, 962.8 μmol) was added and resulting solution was allowed to warm to RT overnight. The reaction mixture was diluted with H₂O (25 mL) and EtOAc (25 mL) and the aqueous layer was extracted with EtOAc (25 mL). The combined organic layers were washed with H₂O (25 mL×3), sat. aq. NaCl (25 mL), dried over anhydrous Na₂SO₄, filtered, and concentrated under reduced pressure. The crude product was purified by flash silica gel chromatography eluting with 0-80% 3:1 EtOAc:EtOH in heptane to give the title compound (290.9 mg, 86%) as a white solid. ES-MS m/z 750.4/752.4 (M+H).

### Preparation 444

### (R)-2-((1S,3R)-1-(4-bromo-2-chlorophenyl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide

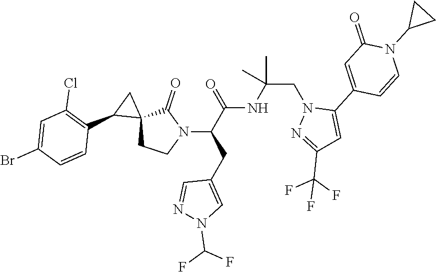 [SMILES: CC(C)(Cn1nc(C(F)(F)F)cc1-c1ccn(C2CC2)c(=O)c1)NC(=O)[C@@H](Cc1cnn(C(F)F)c1)N1CC[C@@]2(C[C@@H]2c2ccc(Br)cc2Cl)C1=O]

To a mixture of 4-(1-(2-amino-2-methylpropyl)-3-(trifluoromethyl)-1H-pyrazol-5-yl)-1-cyclopropylpyridin-2(1H)-one hydrochloride (675 mg, 1.70 mmol) and (R)-2-((1R,3S)-1-(4-bromo-2-chlorophenyl)-4-oxo-5-azaspiro[2.4]heptan-5-yl)-3-(1-(difluoromethyl)-1H-pyrazol-4-yl)propanoic acid (571 mg, 1.17 mmol) in DMA (6.0 mL), was added 4-methylmorpholine (0.65 mL, 5.9 mmol). The reaction mixture was cooled to 0° C., then diethyl (4-oxo-1,2,3-benzotriazin-3-yl)phosphate (719 mg, 2.40 mmol) was added and the resulting solution was allowed to warm to RT over 5 h. The reaction mixture was diluted with H₂O (25 mL) and EtOAc (25 mL), and the aqueous layer was extracted with EtOAc (25 mL×2). The combined organic layers were washed with sat. aq. NaCl (25 mL×3), dried over Na₂SO₄, filtered, and concentrated under reduced pressure. The crude product was purified by on silica, eluting with 0-100% EtOAc in heptane followed by 100% (3:1) EtOAc:EtOH to give the title compound (946.6 mg, 100%) as a yellow solid. ES-MS m/z 810.2/812.2 (M+H).

### Preparation 445

### (R)-2-((trans)-1-(6-bromo-2-cyclopropylpyridin-3-yl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide (mixture of isomers)

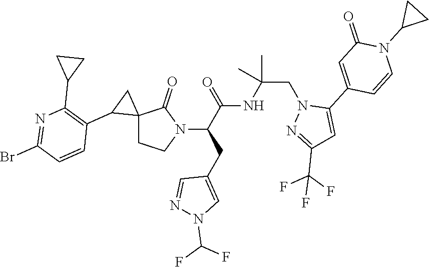 [SMILES: CC(C)(Cn1nc(C(F)(F)F)cc1-c1ccn(C2CC2)c(=O)c1)NC(=O)[C@@H](Cc1cnn(C(F)F)c1)N1CCC2(CC2c2ccc(Br)nc2C2CC2)C1=O]

(trans)-2-(6-bromo-2-cyclopropylpyridin-3-yl)-1-(2-chloroethyl)-N—((R)-1-((1-(5-(1-cyclopropyl-2-oxo-1,2-dihydropyridin-4-yl)-3-(trifluoromethyl)-1H-pyrazol-1-yl)-2-methylpropan-2-yl)amino)-3-(1-(difluoromethyl)-1H-pyrazol-4-yl)-1-oxopropan-2-yl)cyclopropane-1-carboxamide (895 mg, 1.05 mmol, mixture of isomers and 2-bromo and 2-chloropyridyl analogues) and TBAI (77.4 mg, 210 μmol) were dissolved in NMP (10.5 mL). The reaction mixture was stirred at RT overnight, then was filtered over celite and purified on silica gel, eluting with 0-100% 3:1 EtOH/EtOAc in heptanes. The title compound (320 mg, 37% yield) as a yellow oil (isolated as a mixture of 2-bromo and 2-chloropyridyl analogues). ES-MS m/z 773.4, 817.4, 819.4 (M+H).

### Preparation 446

### tert-butyl (1-(5-(dimethylphosphoryl)-3-(trifluoromethyl)-1H-pyrazol-1-yl)-2-methylpropan-2-yl)carbamate

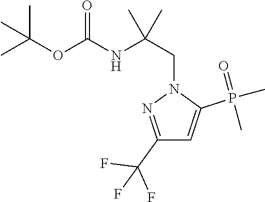 [SMILES: CC(C)(Cn1nc(C(F)(F)F)cc1P(C)(C)=O)NC(=O)OC(C)(C)C]

In a N₂-filled glove box, a solution of dimethylphosphine oxide (1.1 g, 14 mmol) in 1,4-dioxane (10 mL) was charged with tert-butyl (1-(5-bromo-3-(trifluoromethyl)-1H-pyrazol-1-yl)-2-methylpropan-2-yl)carbamate (1.0 g, 2.6 mmol), 1,1′-Ferrocendiylbis(diphenylphosphine) (285 mg, 514 μmol), palladium diacetate (126 mg, 561 μmol) and TEA (908 μL, 6.51 mmol). The reaction mixture was sealed, removed from the glove box, and stirred at 110° C. for 4 h under microwave irradiation. After cooling, the reaction mixture was filtered and concentrated under reduced pressure. Normal-phase chromatography (SiO₂, gradient 0-3% MeOH in CH₂Cl₂) followed by reverse-phase chromatography (C18, gradient 5-50% ACN in 0.5% aqueous FA) provided the title compound (253 mg, 23%) as a yellow solid. ES-MS m/z 284.3 (M-Boc+H).

### Preparation 447

### 1-(5-(3-methoxy-6-methylpyridazin-4-yl)-3-(trifluoromethyl)-1H-pyrazol-1-yl)-2-methylpropan-2-amine

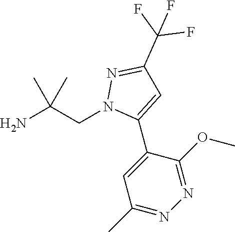 [SMILES: COc1nnc(C)cc1-c1cc(C(F)(F)F)nn1CC(C)(C)N]

tert-butyl (1-(5-(3-methoxy-6-methylpyridazin-4-yl)-3-(trifluoromethyl)-1H-pyrazol-1-yl)-2-methylpropan-2-yl)carbamate (58 mg, 0.14 mmol) and TFA (0.5 mL) were stirred at 25° C. for 0.5 h. The reaction was concentrated, and the resulting material was dissolved in EtOAc (100 mL) and washed with 2% sodium bicarbonate aqueous solution (3× 10 mL). The organic and water extracts were reduced to dryness under a nitrogen stream and the light-yellow powder was further dried under vacuum to give the title compound (45 mg, 100%, 90% purity). ES-MS m z 330.2 (M+H).

### Preparation 448

### tert-Butyl (R)-(1-((1-(5-(6-(2-hydroxypropan-2-yl)pyridin-3-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)carbamate

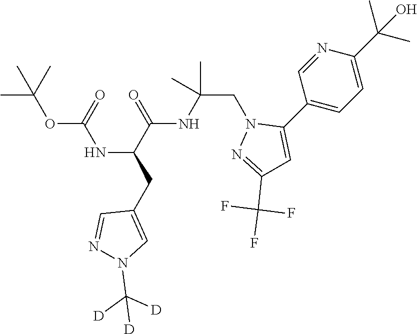 [SMILES: [2H]C([2H])([2H])n1cc(C[C@@H](NC(=O)OC(C)(C)C)C(=O)NC(C)(C)Cn2nc(C(F)(F)F)cc2-c2ccc(C(C)(C)O)nc2)cn1]

(R)-2-((tert-butoxycarbonyl)amino)-3-(1-(methyl-d3)-1H-pyrazol-4-yl)propanoic acid (703 mg, 89% purity, 2.29 mmol) was combined with 2-(5-(1-(2-amino-2-methylpropyl)-3-(trifluoromethyl)-1H-pyrazol-5-yl)pyridin-2-yl)propan-2-ol hydrochloride (550 mg, 1.45 mmol) and HATU (1.39 g, 3.65 mmol) in DMF (6.0 mL) and TEA (1.30 mL, 9.33 mmol). The mixture was stirred at RT for 1 h, then was diluted with 50 mL EtOAc and 50 mL water and the organic layer was separated. The aqueous layer was extracted with EtOAc (50 mL×2). The combined organic layers were washed with sat. aq. NaCl, dried over Na₂SO₄ and concentrated under reduced pressure. The crude product was purified on silica, eluting with 0-60% EtOAc:EtOH (3:1) in heptane to obtain the title compound (1.3 g, 67% purity, 97%) as a yellow oil. ES-MS m/z 597.4 (M+H).

### Preparation 449

### (R)-2-amino-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide hydrochloride

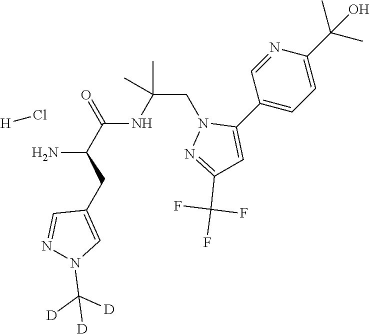

Tert-butyl (R)-(1-((1-(5-(6-(2-hydroxypropan-2-yl)pyridin-3-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)carbamate (1.3 g, 67% purity, 1.4 mmol) was dissolved in EtOH (6.0 mL) and 4 M HCl in 1,4-dioxane (3.0 mL, 10 mmol) was added. The mixture was stirred at RT overnight then concentrated to give quantitative yield of the title compound as a pink gel. ES-MS m/z 497.4 (M+H)

### Preparation 450

### (1R,3R)-1-(6-bromo-2-cyclopropylpyridin-3-yl)-5-oxaspiro[2.4]heptan-4-one

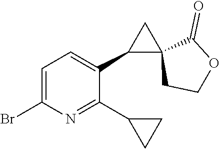 [SMILES: O=C1OCC[C@@]12C[C@@H]2c1ccc(Br)nc1C1CC1]

Rac-(trans)-1-(6-bromo-2-cyclopropylpyridin-3-yl)-5-oxaspiro[2.4]heptan-4-one (4.1 g, 7.09 mmol, 50% purity) was separated by chiral SFC purification (Chiralpak IG (250 mm×30 mm×5 μm, 85 ml/min), eluting with 10% MeOH to afford the title compound (1.03 g, 47%) as the first eluting enantiomer. ES-MS m/z 308.0 (M+H).

### Preparation 451

### Methyl (R)-2-((1R,2S)-2-(4-bromo-2-chlorophenyl)-1-(2-chloroethyl)cyclopropane-1-carboxamido)-3-(1-(methyl-d3)-1H-pyrazol-4-yl)propanoate

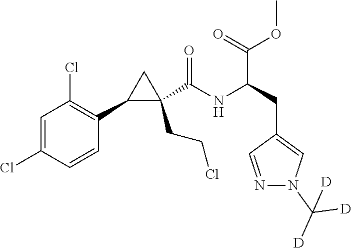 [SMILES: [2H]C([2H])([2H])n1cc(C[C@@H](NC(=O)[C@@]2(CCCl)C[C@@H]2c2ccc(Cl)cc2Cl)C(=O)OC)cn1]

Methyl (R)-2-amino-3-(1-(methyl-d3)-1H-pyrazol-4-yl)propanoate hydrochloride (2.85 g, 12.8 mmol), DCM (50 mL), and TEA (14 mL, 0.10 mol) were combined and cooled to 0° C., then a solution of (1R,2R)-2-(4-bromo-2-chlorophenyl)-1- (2-chloroethyl)cyclopropane-1-carbonyl chloride (4.74 g, 13.3 mmol) in DCM (24 mL) was added dropwise. The reaction mixture was stirred at 0° C. for 15 min, quenched with half-saturated aq. NH₄Cl (100 mL), stirred at 0° C. for 90 min, the organic layer was collected, and the aqueous was extracted with CH₂Cl₂ (50 mL×3). The combined organics were concentrated at 25° C. under reduced pressure. The residue was transferred to a 200 mL recovery flask with ACN and further concentrated to provide a quantitative yield of the title compound (12.4 g, 100%) as an orange oil. ES-MS m/z 505.0 (M+H).

### Preparation 452

### (1R,2R)-2-(6-chloro-2-cyclopropylpyridin-3-yl)-1-(2-chloroethyl)-N—((R)-1-((1-(5-(6-(2-hydroxypropan-2-yl)pyridin-3-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)cyclopropane-1-carboxamide

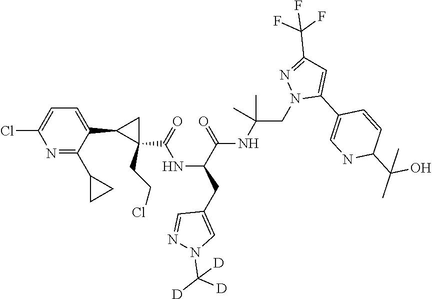 [SMILES: [2H]C([2H])([2H])n1cc(C[C@@H](NC(=O)[C@@]2(CCCl)C[C@@H]2c2ccc(Cl)nc2C2CC2)C(=O)NC(C)(C)Cn2nc(C(F)(F)F)cc2C2=CNC(C(C)(C)O)C=C2)cn1]

Prepared according to Preparation 451_from (1R,3R)-1-(6-bromo-2-cyclopropylpyridin-3-yl)-5-oxaspiro[2.4]heptan-4-one (1.04 g, 3.37 mmol) and (R)-2-amino-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide hydrochloride (750 mg, 1.41 mmol). ES-MS m/z 778.4/780.4 (M+H) (Cl^{35/37}).

### Preparation 453

### methyl (R)-2-((1S,3R)-1-(4-bromo-2-chlorophenyl)-4-oxo-5-azaspiro[2.4]heptan-5-yl)-3-(1-(methyl-d3)-1H-pyrazol-4-yl)propanoate

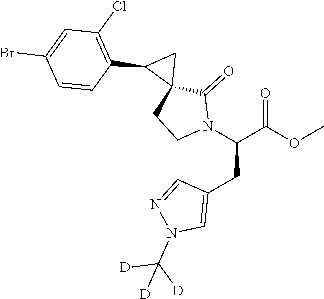 [SMILES: [2H]C([2H])([2H])n1cc(C[C@H](C(=O)OC)N2CC[C@@]3(C[C@@H]3c3ccc(Br)cc3Cl)C2=O)cn1]

Methyl (R)-2-((1R,2S)-2-(4-bromo-2-chlorophenyl)-1-(2-chloroethyl)cyclopropane-1-carboxamido)-3-(1-(methyl-d3)-1H-pyrazol-4-yl)propanoate (12.4 g, 52% purity, 12.7 mmol) was dissolved in ACN (65 mL), and cesium carbonate (9.97 g, 2.40 Eq, 30.6 mmol) was added. The reaction mixture was stirred at RT under Ar overnight. The reaction mixture was concentrated, then diluted with DCM (50 mL) and water (50 mL). HCl (2.6 g, 6.0 mL, 12 molar, 72 mmol) was added dropwise and the mixture was allowed to stir for 30 min at RT. The layers were separated, the organic layer was collected, and the aqueous was extracted with additional DCM (50 mL×3). The combined organic layers were concentrated and purified on silica, eluting with 30-100% EtOAc in heptane to obtain the title compound (5.4 g, 74% purity, 67%). ES-MS m/z 469.0 (M+H).

### Preparation 454

### (R)-2-((1R,3R)-1-(6-chloro-2-cyclopropylpyridin-3-yl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide

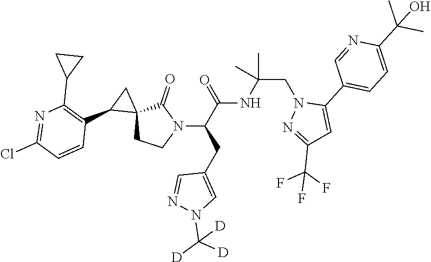 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(Cl)nc3C3CC3)C2=O)cn1]

To (1R,2R)-2-(6-chloro-2-cyclopropylpyridin-3-yl)-1-(2-chloroethyl)-N—((R)-1-((1-(5-(6-(2-hydroxypropan-2-yl)pyridin-3-yl)-3-(trifluoromethyl)-1H-pyrazol-1-yl)-2-methylpropan-2-yl)amino)-3-(1-(methyl-d3)-1H-pyrazol-4-yl)-1-oxopropan-2-yl)cyclopropane-1-carboxamide (511 mg, 70.6% purity, 463 μmol) in NMP (5.0 mL) was added Cs₂CO₃ (654 mg, 2.01 mmol) and the mixture was stirred at RT overnight. The reaction mixture was partitioned between H₂O (20 mL) and EtOAc (20 mL) and the aqueous layer was extracted with EtOAc (20 mL×2). The organic layers were combined, washed with sat. aq. NaCl (20 mL×2), dried over Na₂SO₄, and concentrated under reduced pressure to give the title compound (344 mg, 100%) as a yellow gel. ES-MS m/z 742.4/744.4 (M+H) (Cl^{35/37}).

### Preparation 455

### Lithium (R)-2-((1S,3R)-1-(4-bromo-2-chlorophenyl)-4-oxo-5-azaspiro[2.4]heptan-5-yl)-3-(1-(methyl-d3)-1H-pyrazol-4-yl)propanoate

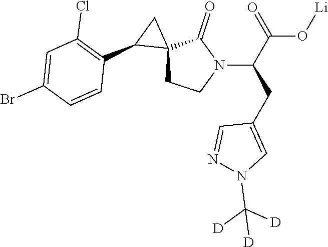

Methyl (R)-2-((1S,3R)-1-(4-bromo-2-chlorophenyl)-4-oxo-5-azaspiro[2.4]heptan-5-yl)-3-(1-(methyl-d3)-1H-pyrazol-4-yl)propanoate (5.4 g, 74% purity, 8.5 mmol) was added tBuOH (40 mL) and diluted with H₂O (10 mL). The reaction mixture was cooled to 0° C. and aqueous lithium hydroxide (5.0 mL, 2.0 M, 10 mmol) was added dropwise at 0° C. over 5 min. The reaction was stirred at 0° C. for 2 h, quenched with 1 M aq. HCl (1.9 mL, 3.8 mmol) dropwise until pH ˜5-6. The mixture was concentrated under reduced pressure and lyophilized to provide a quantitative yield of the title compound (4.3 g, 100%) as a white solid. ES-MS m z 457.0 (M+H).

### Preparation 456

### (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(methyl-d3)-1H-pyrazol-4-yl)propanoic acid

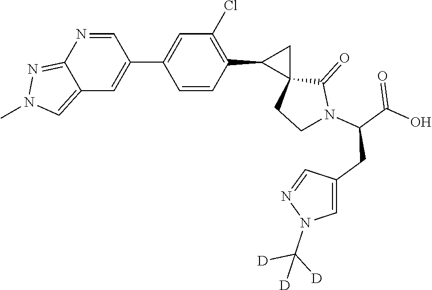 [SMILES: [2H]C([2H])([2H])n1cc(C[C@H](C(=O)O)N2CC[C@@]3(C[C@@H]3c3ccc(-c4cnc5nn(C)cc5c4)cc3Cl)C2=O)cn1]

Lithium (R)-2-((1S,3R)-1-(4-bromo-2-chlorophenyl)-4-oxo-5-azaspiro[2.4]heptan-5-yl)-3-(1-(methyl-d3)-1H-pyrazol-4-yl)propanoate (1.975 g, 3.936 mmol), (2-methyl-2H-pyrazolo[3,4-b]pyridin-5-yl) boronic acid (768 mg, 4.34 mmol), (A-taPhos) 2PdCl₂ (134.3 mg, 189.7 μmol), and Na₂CO₃ (873 mg, 8.24 mmol) were combined in MeOH (2.0 mL) and the headspace was flushed with Ar. The reaction mixture was stirred at 60° C. under Ar overnight, then was cooled to RT and SiliaMetS thiol resin (1.2 g) was added. The mixture was stirred vigorously at RT for 1 h, then diluted with 10 mL H₂O, partially concentrated to 20 mL total volume and quenched by addition of FA (500 μL, 13.3 mmol) until pH=4. The crude product was purified by reverse phase column chromatography (C18, eluting with 10-70% ACN in 0.1% aq FA) to afford the title compound (2.05 g, 3.93 mmol, 100%) as a yellow oil. ES-MS m z 508.2/510.2 (M+H).

### Preparation 457

### (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)-N-(1-(5-(3-methoxy-6-methylpyridazin-4-yl)-3-(trifluoromethyl)-1H-pyrazol-1-yl)-2-methylpropan-2-yl)propanamide

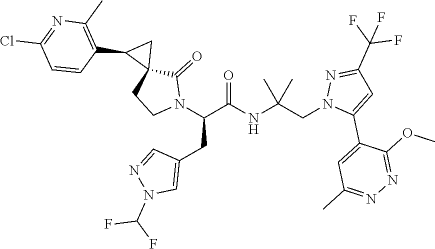 [SMILES: COc1nnc(C)cc1-c1cc(C(F)(F)F)nn1CC(C)(C)NC(=O)[C@@H](Cc1cnn(C(F)F)c1)N1CC[C@@]2(C[C@@H]2c2ccc(Cl)nc2C)C1=O]

To lithium (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)propanoate (50 mg, 0.12 mmol) in DMF (1.6 mL) at 2° C. was added DIPEA (100 μL, 574 μmol) and then HATU (55 mg, 0.14 mmol). To this solution was added in one portion 1-(5-(3-methoxy-6-methylpyridazin-4-yl)-3-(trifluoromethyl)-1H-pyrazol-1-yl)-2-methylpropan-2-amine (44 mg, 0.13 mmol). The resulting yellow solution was stirred for 0.5 h at 2° C., then was poured into 10 mL of water to form a precipitate that was captured by Buchner filtration. The solid was washed with cold water (2×5 mL). This material was dried on the filter cake under house vacuum then at 45° C. for 3 h at 1 mm Hg to obtain the title compound (56 mg, 61%) as a brown solid. ES-MS m/z 736.4 (M+H).

### Preparation 458

### (R)-2-((1R,3R)-1-(6-chloro-2-methylpyridin-3-yl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide

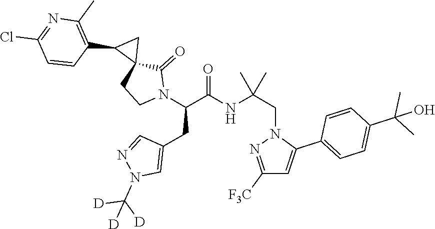 [SMILES: [2H]C([2H])([2H])n1cc(C[C@H](C(=O)NC(C)(C)Cn2nc(C(F)(F)F)cc2-c2ccc(C(C)(C)O)cc2)N2CC[C@@]3(C[C@@H]3c3ccc(Cl)nc3C)C2=O)cn1]

A mixture of lithium (R)-2-((1R,3R)-1-(6-chloro-2-methylpyridin-3-yl)-4-oxo-5-azaspiro[2.4]heptan-5-yl)-3-(1-(methyl-d3)-1H-pyrazol-4-yl)propanoate (299 mg, 692 μmol), 2-(5-(1-(2-amino-2-methylpropyl)-3-(trifluoromethyl)-1H-pyrazol-5-yl)pyridin-2-yl)propan-2-ol dihydrochloride (383 mg, 922 μmol), DMA (3.46 mL), N-methylmorpholine (300 μL, 2.73 mmol), and DEPBT (419 mg, 1.40 mmol) was stirred at RT overnight. The mixture was diluted with H₂O (1.5 mL) and the suspension was loaded onto celite and purified by reverse-phase flash chromatography (C18, gradient 10-100% ACN in 10 mM aqueous NH₄HCO₃₊₅% MeOH) to give the title compound (490 mg, 98.9%) as a honey-colored glass. ES-MS m/z 716.4/718.4 (M+H) (Cl^{35/37}).

The compounds in the following table were prepared as described in Preparation 458 using the appropriate amine and carboxylic acid. Reactants can be added in different orders or in differing equivalency and differing methods can be used to work up or purify the compounds (normal phase, or high, neutral or low pH reversed phase). Such variances would be apparent to one skilled in the art.

TABLE 48

| Prep- | | | ES-MS |
|---|---|---|---|
| aration | Chemical Name | Structure | m/z |
| 459 | (R)-2-((1R,3R)-1-(6- chloro-2- methylpyridin- 3-yl)-4-oxo-5- azaspiro[2.4] heptan-5- yl)-N-(1-(5-(2-(2- hydroxypropan-2- yl)pyrimidin-5-yl)-3- (trifluoromethyl)-1H- pyrazol-1-yl)-2- methylpropan-2-yl)-3- (1-(methyl-d3)-1H- pyrazol-4- yl)propanamide | 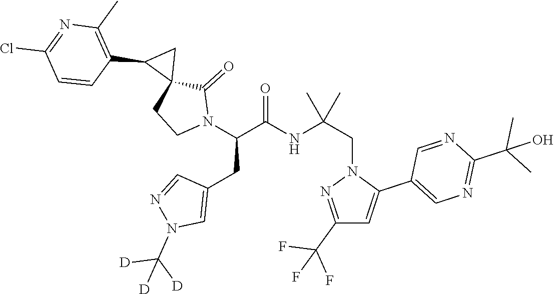 [SMILES: [2H]C([2H])([2H])n1cc(C[C@H](C(=O)NC(C)(C)Cn2nc(C(F)(F)F)cc2-c2cnc(C(C)(C)O)nc2)N2CC[C@@]3(C[C@@H]3c3ccc(Cl)nc3C)C2=O)cn1] | 717.6 (M + H) |
| 460 | (R)-2-((1S,3R)-1-(4- bromo-2- chlorophenyl)- 4-oxo-5- azaspiro[2.4] heptan-5- yl)-N-(1-(5-(2-(2- hydroxypropan-2- yl)pyrimidin-5-yl)-3- (trifluoromethyl)-1H- pyrazol-1-yl)-2- methylpropan-2-yl)-3- (1-(methyl-d3)-1H- pyrazol-4- yl)propanamide | 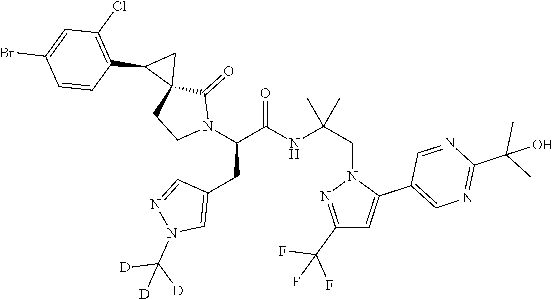 [SMILES: [2H]C([2H])([2H])n1cc(C[C@H](C(=O)NC(C)(C)Cn2nc(C(F)(F)F)cc2-c2cnc(C(C)(C)O)nc2)N2CC[C@@]3(C[C@@H]3c3ccc(Br)cc3Cl)C2=O)cn1] | 780.2 (M + H) |
| 461 | (R)-2-((1S,3R)-1-(4- bromo-2- chlorophenyl)- 4-oxo-5- azaspiro[2.4] heptan-5- yl)-N-(1-(5-(6-(2- hydroxypropan-2- yl)pyridin-3-yl)-3- (trifluoromethyl)-1H- pyrazol-1-yl)-2- methylpropan-2-yl)-3- (1-(methyl-d3)-1H- pyrazol-4- yl)propanamide | 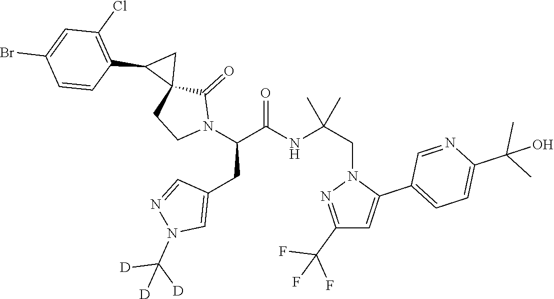 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(Br)cc3Cl)C2=O)cn1] | 779.2 (M + H) |

### Example 11

### (trans)-2-Methyl-N—((R)-1-((2-methyl-1-(4-(trifluoromethyl)-1H-pyrazol-1-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide, Isomer 4

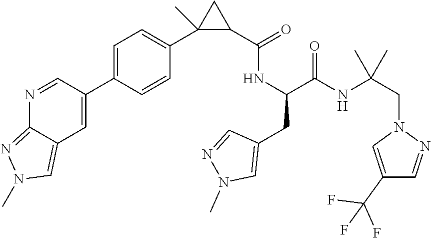 [SMILES: Cn1cc(C[C@@H](NC(=O)C2CC2(C)c2ccc(-c3cnc4nn(C)cc4c3)cc2)C(=O)NC(C)(C)Cn2cc(C(F)(F)F)cn2)cn1]

A solution of rac-2-methyl-2-(4-(2-methyl-2H-pyrazolo[3,4-b]pyridin-5-yl)phenyl)cyclopropane-1-carboxylic acid (mixture of cis/trans enantiomeric pairs, 250 mg, 732 μmol, 90% purity) in DMA (6 mL) was treated with (R)-2-amino-N-(2-methyl-1-(4-(trifluoromethyl)-1H-pyrazol-1-yl)propan-2-yl)-3-(1-methyl-1H-pyrazol-4-yl)propanamide hydrochloride (321 mg, 732 μmol, 90% purity), HATU (418 mg, 1.10 mmol) and DIEA (510 μL, 2.93 mmol). After stirring at 18° C. for 2 h, the reaction mixture was filtered, and the filtrate was concentrated under reduced pressure. The residue was purified by reversed phase prep-HPLC (PrePulite XP tC18, 40×200 mm, 7 μm, column temp: RT; flow rate: 60 mL/min, 200 mg/injection) eluting with 18%-58% ACN in (10 mM aq. NH₄HCO₃ with 0.05% NH₄OH) over 20 min to give the first eluting isomer. The remaining mixture of 3 diastereomers (252 mg) was further purified by prep-SFC (CHIRALCEL® OD, 30×250 mm, 10 μm, column temp: 40° C., flow rate: 80 mL/min, 8×2.5 mL injections) eluting with 35% EtOH (with 0.1% NH₄OH): 65% CO₂ to obtain the title compound (last eluting isomer, 64.1 mg, 13.5%) as a white solid. 96.7% de. ES-MS m/z 648 (M+H).

The compounds in the following table were prepared as described in Example 11 using the appropriate carboxylic acid or carboxylate salt and the appropriate amine or amine salt. Reactants can be added in different orders or in differing equivalency. DMF is a suitable substitute for DMA. Reaction times (0.5 h to overnight) and temperatures (0° C. to RT) may vary, and differing methods can be used to work up (can be directly concentrated or partitioned between EtOAc/water) or purify the compounds (such as normal phase or high, neutral or low pH prep-HPLC). Such variances would be apparent to one skilled in the art.

TABLE 29

| Ex- | | | |
|---|---|---|---|
| am- | | | ES-MS |
| ple | Chemical Name | Structure | m/z |
| 12 | (1S,2S)-N- ((R)-1-((1-(5- bromo-3- (trifluoromethyl)- 1H-pyrazol-1-yl)-2- methylpropan- 2-yl)amino)- 3-(1-(difluoromethyl)- 1H-pyrazol-4-yl)- 1-oxopropan- 2-yl)-2-(4- (2-methyl-2H- pyrazolo[3,4-b] pyridin-5- yl)phenyl) cyclopropane-1- carboxamide | 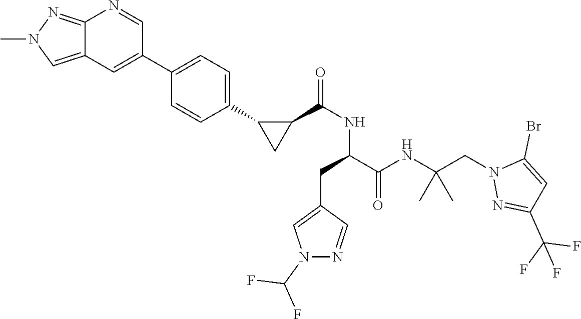 [SMILES: Cn1cc2cc(-c3ccc([C@H]4C[C@@H]4C(=O)N[C@H](Cc4cnn(C(F)F)c4)C(=O)NC(C)(C)Cn4nc(C(F)(F)F)cc4Br)cc3)cnc2n1] | 748, 750 (M + H) |
| 13^{a} | (1S,2S)-N- ((R)-1-((1-(5-(2H- 1,2,3-Triazol-2-yl)-3- (trifluoromethyl)-1H- pyrazol-1-yl)-2- methylpropan- 2-yl)amino)- 3-(1-methyl- 1H-pyrazol-4- yl)-1-oxopropan- 2-yl)-2-(4- (2-methyl-2H- pyrazolo[3,4- b]pyridin-5- yl)phenyl) cyclopropane- 1-carboxamide | 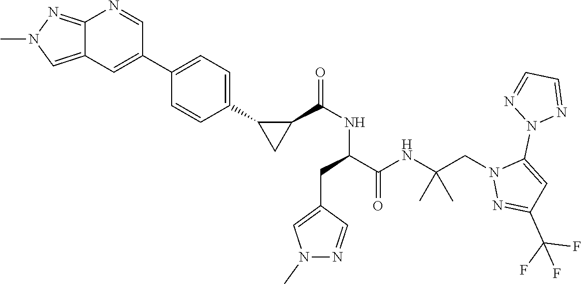 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-n2nccn2)cn1] | 701.4 (M + H) |
| 14 | (1S,2S)-N- ((R)-1-((1-(4- Fluoro-5′- (trifluoromethyl)- 1H,2′H-[3,3′- bipyrazol]-2′- yl)-2-methylpropan-2- yl)amino)-3- (1-methyl-1H- pyrazol-4-yl)- 1-oxopropan- 2-yl)-2-(4- (2-methyl-2H- pyrazolo[3,4- b]pyridin-5- | 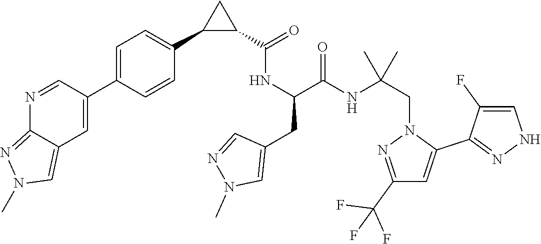 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2n[nH]cc2F)cn1] | 718.3 (M + H) |
| | yl)phenyl) | | |
| | cyclopropane- | | |
| | 1-carboxamide | | |
| 15 | (1S,2S)-N-((R)- 1-((2-Methyl- 1-(5-(2-methyl- 1H-imidazol- 5-yl)-3- (trifluoromethyl)- 1H-pyrazol-1- yl)propan-2- yl)amino)-3- (1-methyl-1H- pyrazol-4-yl)- 1-oxopropan- 2-yl)-2-(4- (2-methyl-2H- pyrazolo[3,4-b] pyridin-5- | 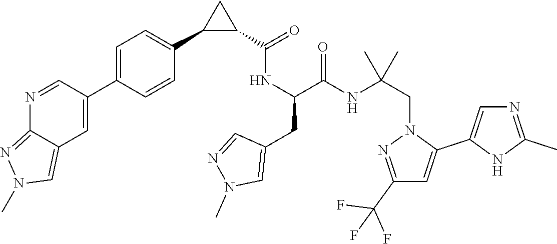 | 714.4 (M + H) |
| | yl)phenyl) | | |
| | cyclopropane- | | |
| | 1-carboxamide | | |
| 16 | (1S,2S)-N- ((R)-1-((2-Methyl- 1-(5-(5-oxo- 4,5-dihydro- 1,3,4-thiadiazol- 2-yl)-3- (trifluoromethyl)-1H- pyrazol-1-yl)propan-2- yl)amino)-3- (1-methyl-1H- pyrazol-4-yl)- 1-oxopropan- 2-yl)-2-(4- (2-methyl-2H- pyrazolo[3,4-b] | 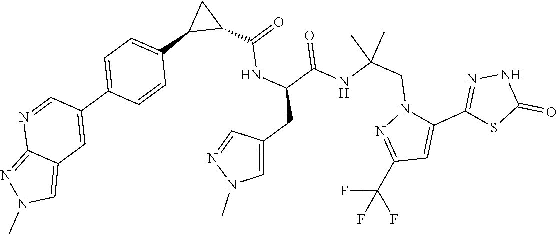 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2n[nH]c(=O)s2)cn1] | 734.2 (M + H) |
| | pyridin-5- | | |
| | yl)phenyl) | | |
| | cyclopropane-1- | | |
| | carboxamide | | |
| 17 | (1S,2S)-N- ((R)-1-((1-(5- (1,2,4-Thiadiazol- 3-yl)-3- (trifluoromethyl)-1H- pyrazol-1-yl)-2- methylpropan- 2-yl)amino)- 3-(1-methyl- 1H-pyrazol-4- yl)-1-oxopropan- 2-yl)-2-(4- (2-methyl-2H-pyrazolo [3,4-b]pyridin-5- yl)phenyl) | 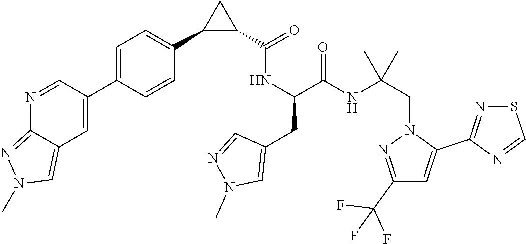 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2ncsn2)cn1] | 718.4 (M + H) |
| | cyclopropane- | | |
| | 1-carboxamide | | |
| 18 | (1S,2S)-N- ((R)-1-((1-(5-(2- (2-Hydroxypropan-2- yl)pyridin-4-yl)-3- (trifluoromethyl)-1H- pyrazol-1-yl)-2- methylpropan- 2-yl)amino)- 3-(1-methyl- 1H-pyrazol-4- yl)-1-oxopropan- 2-yl)-2-(4-(2- methyl-2H-pyrazolo [3,4-b]pyridin-5- yl)phenyl) cyclopropane- 1-carboxamide | 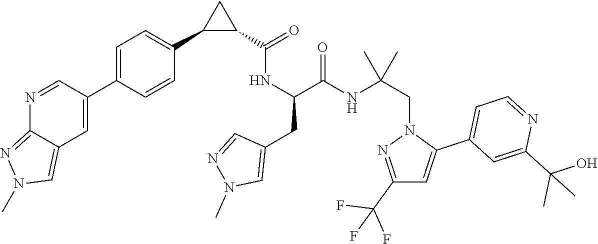 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2ccnc(C(C)(C)O)c2)cn1] | 769.4 (M + H) |
| 19 | (1S,2S)-N-((R)- 1-((2-Methyl- 1-(5-(1-methyl- 6-oxo-1,6- dihydropyridin- 3-yl)-3- (trifluoromethyl)- 1H-pyrazol-1- yl)propan-2- yl)amino)-3- (1-methyl-1H- pyrazol-4-yl)- 1-oxopropan- 2-yl)-2-(4-(2- methyl-2H- pyrazolo[3,4- | 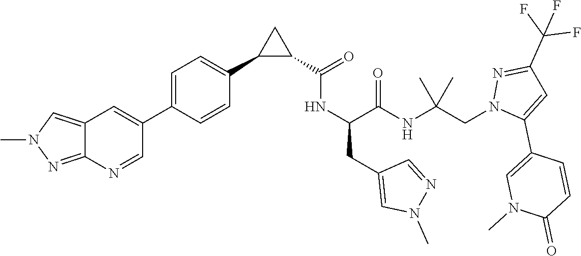 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2ccc(=O)n(C)c2)cn1] | 741.4 (M + H) |
| | b]pyridin-5- | | |
| | yl)phenyl) | | |
| | cyclopropane- | | |
| | 1-carboxamide | | |
| 20 | (1S,2S)-N-((R)- 1-((2-Methyl- 1-(5-(2- oxopyridin-1(2H)- yl)-3- (trifluoromethyl)-1H- pyrazol-1-yl)propan- 2-yl)amino)-3- (1-methyl-1H- pyrazol-4-yl)- 1-oxopropan- 2-yl)-2-(4- (2-methyl-2H- pyrazolo[3,4- b]pyridin-5- yl)phenyl) cyclopropane- 1-carboxamide | 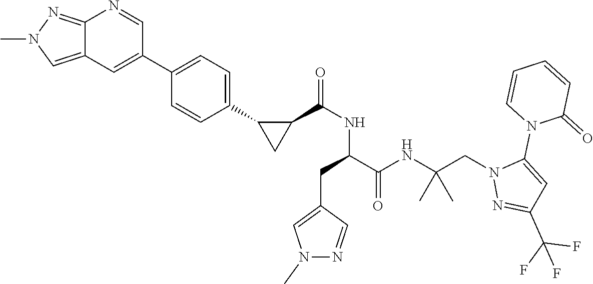 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-n2ccccc2=O)cn1] | 727.2 (M + H) |
| 21 | (1S,2S)-N-((R)-1-((1- (5-Cyclopropyl-3- (trifluoromethyl)-1H- pyrazol-1-yl)-2- methylpropan- 2-yl)amino)- 3-(1-methyl- 1H-pyrazol-4- yl)-1-oxopropan- 2-yl)-2-(4-(2- methyl-2H-pyrazolo [3,4-b]pyridin-5- yl)phenyl) cyclopropane- 1-carboxamide | 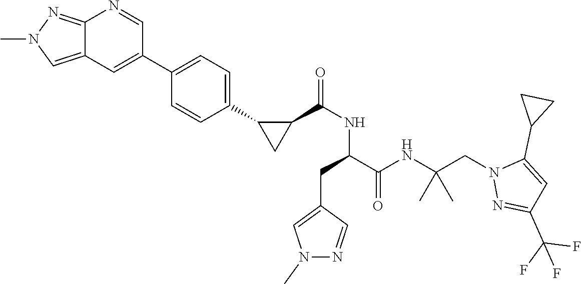 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2C2CC2)cn1] | 674.6 (M + H) |
| 22 | (1S,2S)-N-((R)- 1-((2-Methyl-1-(4- (trifluoromethyl)-1H- pyrazol-1-yl)propan- 2-yl)amino)-3- (1-methyl-1H- pyrazol-4-yl)- 1-oxopropan- 2-yl)-2-(6- (2-methyl-2H- pyrazolo[3,4- b]pyridin-5-yl)- 2-(trifluoromethyl) pyridin-3- yl)cyclopropane-1- carboxamide | 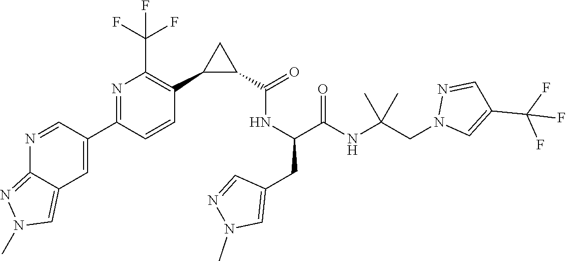 [SMILES: Cn1cc(C[C@@H](NC(=O)[C@H]2C[C@@H]2c2ccc(-c3cnc4nn(C)cc4c3)nc2C(F)(F)F)C(=O)NC(C)(C)Cn2cc(C(F)(F)F)cn2)cn1] | 703.6 (M + H) |
| 23 | (1S,2S)-N-((R)- 1-((1-(5-(1H- 1,2,3-Triazol-1-yl)-3- (trifluoromethyl)-1H- pyrazol-1-yl)-2- methylpropan- 2-yl)amino)- 3-(1-methyl- 1H-pyrazol-4- yl)-1-oxopropan-2- yl)-2-(4-(2- (trifluoromethyl) imidazo [1,2-a] pyrimidin-6- yl)phenyl) cyclopropane- 1-carboxamide | 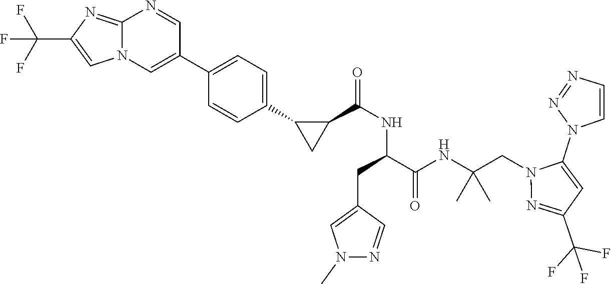 [SMILES: Cn1cc(C[C@@H](NC(=O)[C@H]2C[C@@H]2c2ccc(-c3cnc4nc(C(F)(F)F)cn4c3)cc2)C(=O)NC(C)(C)Cn2nc(C(F)(F)F)cc2-n2ccnn2)cn1] | 755.4 (M + H) |
| 24 | Methyl 1-(2- methyl-2-((R)- 3-(1-methyl- 1H-pyrazol-4- yl)-2-((15,2S)-2-(4-(2- (trifluoromethyl) imidazo [1,2-a]pyrimidin-6- yl)phenyl) cyclopropane-1- carboxamido) propanamido) propyl)-3- (trifluoromethyl)- 1H-pyrazole- 5-carboxylate | 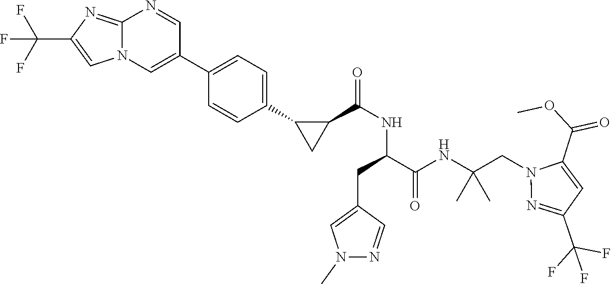 [SMILES: COC(=O)c1cc(C(F)(F)F)nn1CC(C)(C)NC(=O)[C@@H](Cc1cnn(C)c1)NC(=O)[C@H]1C[C@@H]1c1ccc(-c2cnc3nc(C(F)(F)F)cn3c2)cc1] | 746.2 (M + H) |
| 25 | (1S,2S)-N-((R)- 1-((1-(5-(4H- 1,2,4-Triazol-3-yl)-3- (trifluoromethyl)-1H- pyrazol-1-yl)-2- methylpropan- 2-yl)amino)- 3-(1-methyl- 1H-pyrazol-4- yl)-1-oxopropan-2-yl)- 2-(4-(2- (trifluoromethyl) imidazo [1,2-a]pyrimidin-6- yl)phenyl) cyclopropane- 1-carboxamide | 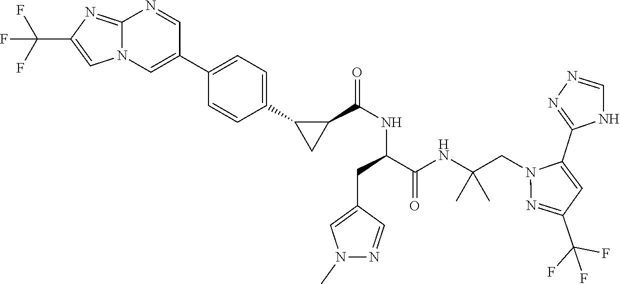 [SMILES: Cn1cc(C[C@@H](NC(=O)[C@H]2C[C@@H]2c2ccc(-c3cnc4nc(C(F)(F)F)cn4c3)cc2)C(=O)NC(C)(C)Cn2nc(C(F)(F)F)cc2-c2nnc[nH]2)cn1] | 755.2 (M + H) |
| 26 | (1S,2S)-N-((R)- 1-((1-(5-(2H- 1,2,3-Triazol-4-yl)-3- (trifluoromethyl)-1H- pyrazol-1-yl)-2- methylpropan- 2-yl)amino)- 3-(1-methyl- 1H-pyrazol-4- yl)-1-oxopropan-2-yl)- 2-(4-(2- (trifluoromethyl) imidazo [1,2-a]pyrimidin-6- yl)phenyl) cyclopropane- 1-carboxamide | 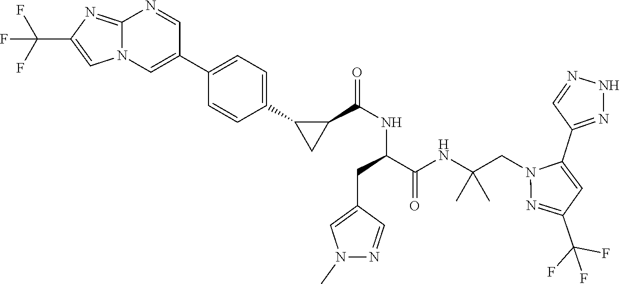 [SMILES: Cn1cc(C[C@@H](NC(=O)[C@H]2C[C@@H]2c2ccc(-c3cnc4nc(C(F)(F)F)cn4c3)cc2)C(=O)NC(C)(C)Cn2nc(C(F)(F)F)cc2-c2cn[nH]n2)cn1] | 755.2 (M + H) |
| 27 | (1S,2S)-N-((R)- 1-((1-(5-(2H- 1,2,3-Triazol-4-yl)-3- (trifluoromethyl)-1H- pyrazol-1-yl)-2- methylpropan- 2-yl)amino)- 3-(1-methyl- 1H-pyrazol-4- yl)-1-oxopropan- 2-yl)-2-(4- (imidazo[1,2-a] pyrimidin-6- yl)phenyl) cyclopropane- 1-carboxamide | 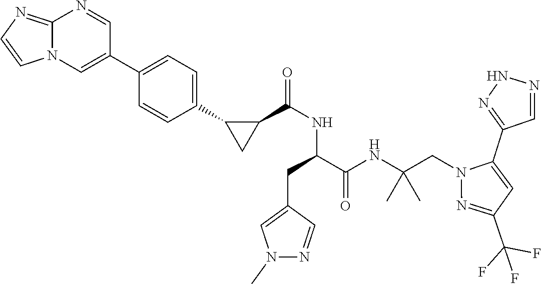 [SMILES: Cn1cc(C[C@@H](NC(=O)[C@H]2C[C@@H]2c2ccc(-c3cnc4nccn4c3)cc2)C(=O)NC(C)(C)Cn2nc(C(F)(F)F)cc2-c2cn[nH]n2)cn1] | 687.2 (M + H) |
| 28 | (1S,2S)-2-(4- (Imidazo[1,2- a]pyrimidin-6- yl)phenyl)-N- ((R)-1-((2-methyl- 1-(1′-methyl-5- (trifluoromethyl)- 1′H,2H-[3,4′- bipyrazol]-2- yl)propan-2- yl)amino)-3-(1- methyl-1H- pyrazol-4-yl)- 1-oxopropan-2- yl)cyclopropane-1- carboxamide | 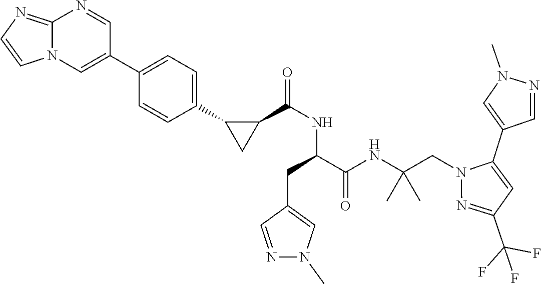 [SMILES: Cn1cc(C[C@@H](NC(=O)[C@H]2C[C@@H]2c2ccc(-c3cnc4nccn4c3)cc2)C(=O)NC(C)(C)Cn2nc(C(F)(F)F)cc2-c2cnn(C)c2)cn1] | 700.4 (M + H) |
| 29^{b} | (trans)-N- ((R)-1-((1-(4- Fluorophenyl)-2- methylpropan- 2-yl)amino)- 3-(1-methyl- 1H-pyrazol-4- yl)-1-oxopropan- 2-yl)-2-(4- (imidazo[1,2-a] pyrimidin-6- yl)-2- (trifluoromethyl) phenyl) cyclopropane-1- carboxamide, Isomer 2 | 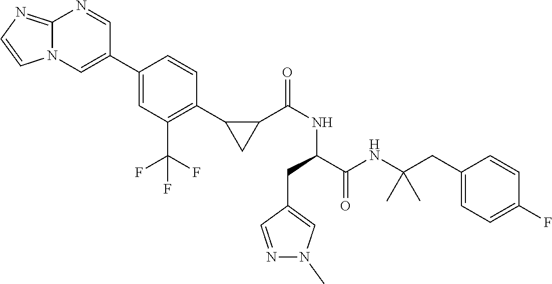 [SMILES: Cn1cc(C[C@@H](NC(=O)C2CC2c2ccc(-c3cnc4nccn4c3)cc2C(F)(F)F)C(=O)NC(C)(C)Cc2ccc(F)cc2)cn1] | 648.2 (M + H) |
| 30 | (1S,2S)-N- Methyl-N-((R)-1- ((2-methyl-1- (5-(5-oxo-4,5- dihydro-1,3,4- thiadiazol-2-yl)-3- (trifluoromethyl)-1H- pyrazol-1-yl) propan-2- yl)amino)-3- (1-methyl-1H- pyrazol-4-yl)- 1-oxopropan- 2-yl)-2-(4- (2-methyl-2H- pyrazolo[3,4- | 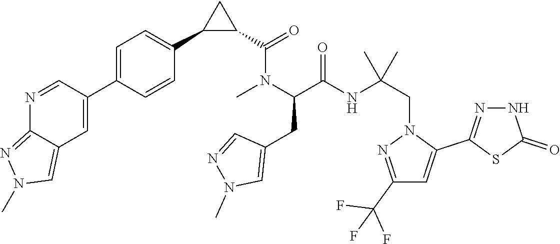 [SMILES: CN(C(=O)[C@H]1C[C@@H]1c1ccc(-c2cnc3nn(C)cc3c2)cc1)[C@H](Cc1cnn(C)c1)C(=O)NC(C)(C)Cn1nc(C(F)(F)F)cc1-c1n[nH]c(=O)s1] | 748.2 (M + H) |
| | b]pyridin-5- | | |
| | yl)phenyl) | | |
| | cyclopropane-1- | | |
| | carboxamide | | |
| 31 | (1S,2S)-N- Methyl-N-((R)-1- ((2-methyl-1- (5-(1-methyl-6- oxo-1,6- dihydropyridin-3- yl)-3- (trifluoromethyl)-1H- pyrazol-1-yl)propan- 2-yl)amino)-3- (1-methyl-1H- pyrazol-4-yl)- 1-oxopropan- 2-yl)-2-(4- (2-methyl-2H- pyrazolo[3,4- b]pyridin-5- yl)phenyl) cyclopropane-1- carboxamide | 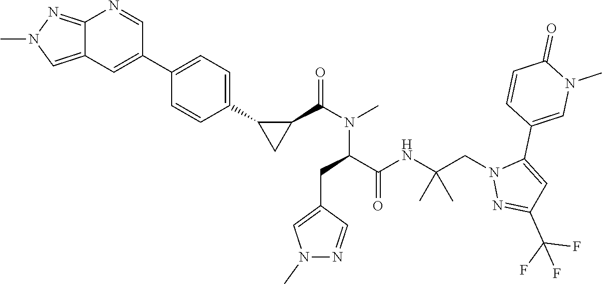 | 755.4 (M + H) |
| 32 | (1S,2S)-N- Methyl-N-((R)-1- ((2-methyl-1- (5-(2-oxo-1,2- dihydropyridin- 3-yl)-3- (trifluoromethyl)-1H- pyrazol-1-yl)propan- 2-yl)amino)-3- (1-methyl-1H- pyrazol-4-yl)- 1-oxopropan- 2-yl)-2-(4-(2- methyl-2H- pyrazolo[3,4- | 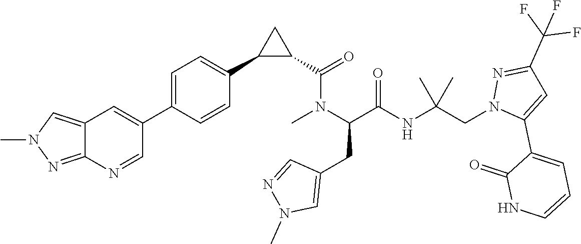 [SMILES: CN(C(=O)[C@H]1C[C@@H]1c1ccc(-c2cnc3nn(C)cc3c2)cc1)[C@H](Cc1cnn(C)c1)C(=O)NC(C)(C)Cn1nc(C(F)(F)F)cc1-c1ccc[nH]c1=O] | 741.2 (M + H) |
| | b]pyridin-5- | | |
| | yl)phenyl) | | |
| | cyclopropane-1- | | |
| | carboxamide | | |
| 33 | (1S,2S)-N- Methyl-N-((R)-1- ((2-methyl-1- (5-(6-oxo-1,6- dihydropyridin- 2-yl)-3- (trifluoromethyl)-1H- pyrazol-1-yl)propan- 2-yl)amino)-3- (1-methyl-1H- pyrazol-4-yl)- 1-oxopropan- 2-yl)-2-(4-(2-methyl- 2H-pyrazolo[3,4- b]pyridin-5- yl)phenyl) cyclopropane-1- carboxamide | 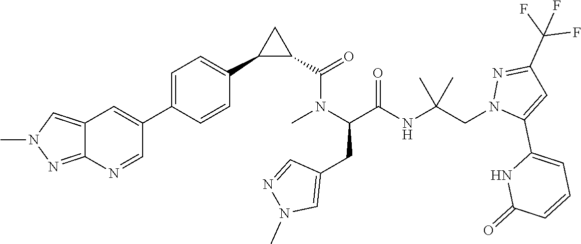 [SMILES: CN(C(=O)[C@H]1C[C@@H]1c1ccc(-c2cnc3nn(C)cc3c2)cc1)[C@H](Cc1cnn(C)c1)C(=O)NC(C)(C)Cn1nc(C(F)(F)F)cc1-c1cccc(=O)[nH]1] | 741.2 (M + H) |
| 34 | (1S,2S)-2- (4-(imidazo[1,2- a]pyrimidin-6- yl)phenyl)-N- methyl-N-((R)- 1-((2-methyl- 1-(5-(1-methyl- 6-oxo-1,6- dihydropyridin- 3-yl)-3- (trifluoromethyl)-1H- pyrazol-1-yl)propan- 2-yl)amino)-3- (1-methyl-1H- pyrazol-4-yl)- 1-oxopropan- 2-yl)cyclopropane-1- carboxamide | 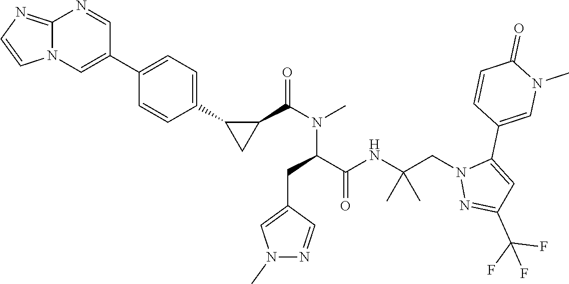 [SMILES: CN(C(=O)[C@H]1C[C@@H]1c1ccc(-c2cnc3nccn3c2)cc1)[C@H](Cc1cnn(C)c1)C(=O)NC(C)(C)Cn1nc(C(F)(F)F)cc1-c1ccc(=O)n(C)c1] | 741.4 (M + H) |
| 35 | 4,4-Difluoro- N-(4-((1S,2S)- 2-(methyl((R)- 1-((2-methyl- 1-(5-(1-methyl- 6-oxo-1,6- dihydropyridin- 3-yl)-3- (trifluoromethyl)-1H- pyrazol-1-yl)propan- 2-yl)amino)-3- (1-methyl-1H- pyrazol-4-yl)-1- oxopropan-2- yl)carbamoyl) cyclopropyl) phenyl)cyclohexane- 1-carboxamide | 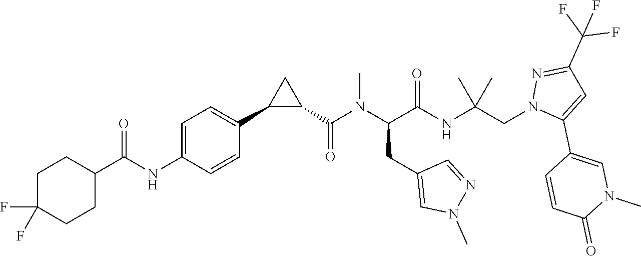 [SMILES: CN(C(=O)[C@H]1C[C@@H]1c1ccc(NC(=O)C2CCC(F)(F)CC2)cc1)[C@H](Cc1cnn(C)c1)C(=O)NC(C)(C)Cn1nc(C(F)(F)F)cc1-c1ccc(=O)n(C)c1] | 785.4 (M + H) |
| 36 | (1S,2S)-N-((R)- 1-((1-(4- Fluorophenyl)-2- methylpropan- 2-yl)amino)- 3-(1-methyl- 1H-pyrazol-4- yl)-1-oxopropan- 2-yl)-2-(4- (3-(4- fluorophenyl) ureido)phenyl)-N- methylcyclopropane- 1-carboxamide | 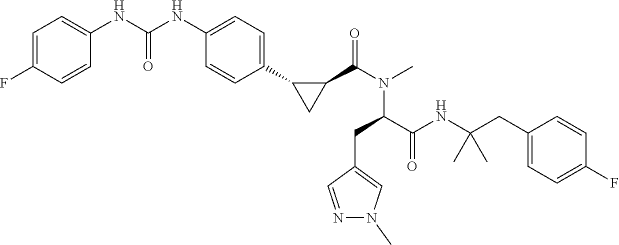 [SMILES: CN(C(=O)[C@H]1C[C@@H]1c1ccc(NC(=O)Nc2ccc(F)cc2)cc1)[C@H](Cc1cnn(C)c1)C(=O)NC(C)(C)Cc1ccc(F)cc1] | 629.4 (M + H) |
| 37 | (1S,2S)-N- ((R)-1-((1-(5- Cyclopropyl-3- (trifluoromethyl)-1H- pyrazol-1-yl)-2- methylpropan- 2-yl)amino)- 3-(imidazo[1,2-a] pyrimidin- 3-yl)-1-oxopropan- 2-yl)-2- (4-(2-methyl-2H- pyrazolo[3,4- b]pyridin-5- yl)phenyl) cyclopropane- 1-carboxamide | 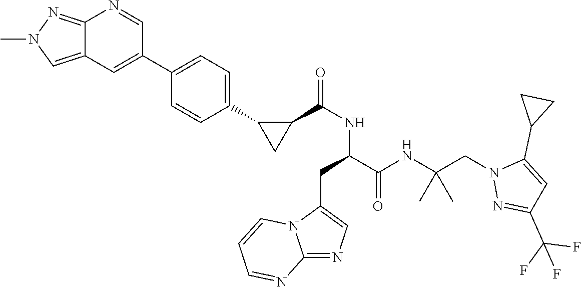 [SMILES: Cn1cc2cc(-c3ccc([C@H]4C[C@@H]4C(=O)N[C@H](Cc4cnc5ncccn45)C(=O)NC(C)(C)Cn4nc(C(F)(F)F)cc4C4CC4)cc3)cnc2n1] | 711.6 (M + H) |
| 38 | (1S,2S)-N-((R)-3- (Imidazo[1,2-a] pyrimidin-3- yl)-1-((2-methyl-1-(1′- methyl-5- (trifluoromethyl)- 1′H,2H-[3,4′-b] pyrazol]-2- yl)propan-2- yl)amino)-1- oxopropan-2-yl)-2-(4- (imidazo[1,2-a] pyrimidin-6- yl)phenyl) cyclopropane- 1-carboxamide | 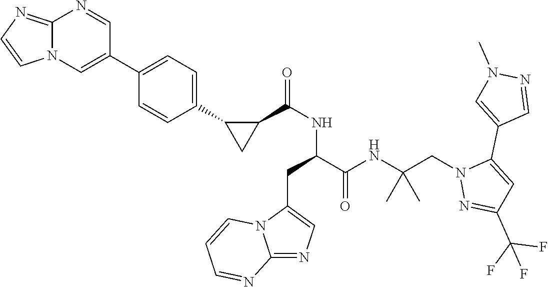 [SMILES: Cn1cc(-c2cc(C(F)(F)F)nn2CC(C)(C)NC(=O)[C@@H](Cc2cnc3ncccn23)NC(=O)[C@H]2C[C@@H]2c2ccc(-c3cnc4nccn4c3)cc2)cn1] | 737.4 (M + H) |
| 39 | (1S,2S)-N-((R)-3- (Imidazo[1,2- a]pyrimidin-3- yl)-1-((2-methyl-1-(3- (trifluoromethyl)-1H- pyrazol-1-yl)propan-2- yl)amino)-1- oxopropan-2- yl)-2-(4-(imidazo[1,2- a]pyrimidin-6- yl)phenyl) cyclopropane- 1-carboxamide | 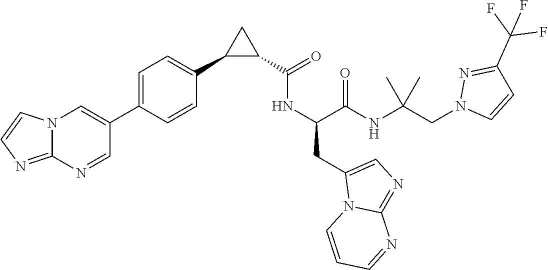 [SMILES: CC(C)(Cn1ccc(C(F)(F)F)n1)NC(=O)[C@@H](Cc1cnc2ncccn12)NC(=O)[C@H]1C[C@@H]1c1ccc(-c2cnc3nccn3c2)cc1] | 657.2 (M + H) |
| 40 | N-(4-((1S,2S)- 2-(((R)-1-((1- (5-cyclopropyl-3- (trifluoromethyl)-1H- pyrazol-1-yl)-2- methylpropan- 2-yl)amino)- 3-(imidazo[1,2-a] pyrimidin- 3-yl)-1-oxopropan-2- yl)carbamoyl) cyclopropyl) phenyl)-3,3- difluorocyclobutane- 1-carboxamide | 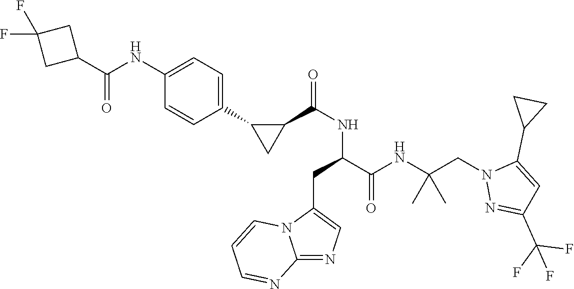 [SMILES: CC(C)(Cn1nc(C(F)(F)F)cc1C1CC1)NC(=O)[C@@H](Cc1cnc2ncccn12)NC(=O)[C@H]1C[C@@H]1c1ccc(NC(=O)C2CC(F)(F)C2)cc1] | 713.6 (M + H) |

^{a}~4:1 mixture of regioisomers carried forward from Preparation 8. Separated by prep-HPLC (Chiralpak ® IC, 30 × 250 mm, 5 μm, column temp: RT, flow rate: 40 mL/min) eluting with 100% MeOH to obtain the first eluting (major) regioisomer as the title compound.
^{b}second eluting isomer when purified by reversed phase prep-HPLC (XSelect ® CSH C18, 50 × 250 mm, 5 μm, 100 mL/min) eluting with 5-95% ACN in in water (with 0.1% FA).

### Example 41

### (1S,2S)-N—((R)-1-((2-Methyl-1-(5-(trifluoromethyl)-1′H,2H-[3,4′-bipyrazol]-2-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

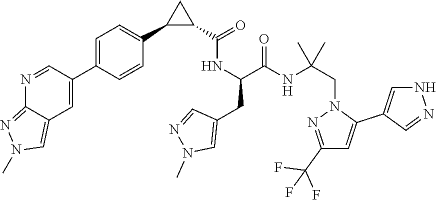 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cn[nH]c2)cn1]

To a solution of (R)-2-amino-N-(2-methyl-1-(5-(trifluoromethyl)-1′H,2H-[3,4′-bipyrazol]-2-yl)propan-2-yl)-3-(1-methyl-1H-pyrazol-4-yl)propanamide hydrochloride (160 mg, 187 μmol, 54% purity) and (1S,2S)-2-(4-(2-methyl-2H-pyrazolo[3,4-b]pyridin-5-yl)phenyl)cyclopropane-1-carboxylic acid (61 mg, 187 μmol, 90 purity %) in pyridine (2.5 mL) was added EDCI (72 mg, 375 μmol), and the resulting mixture was stirred at 18° C. for 16 h. EDCI (36 mg, 187 μmol) was added, and stirring was continued for an additional 16 h. The reaction mixture was diluted with EtOAc (8 mL) and washed with water (2×6 mL) then sat. aq. NaCl (2×7 mL). The organic phase was dried over Na₂SO₄, filtered, and concentrated under reduced pressure. The residue was purified by prep-HPLC (Xtimate® C18, 40×200 mm, 7 μm) eluting with 16-56% ACN in (10 mM aq. NH₄HCO₃ with 0.05% NH₄OH) over 25 min, followed by prep-TLC (10:1 DCM: MeOH, R_{f}=0.4) to obtain the title compound (12 mg, 9%) as a white solid. ES-MS 700.2 m/z (M+H)

The compounds in the following table were prepared as described in Example 41 using the appropriate carboxylic acid or carboxylate salt and the appropriate amine or amine hydrochloride salt. Reactants can be added in different orders or in differing equivalency. Reaction times (3 h to 16 h) may vary, and differing methods can be used to work up or purify the compounds (normal phase, high, neutral or low pH prep-HPLC). Such variances would be apparent to one skilled in the art.

TABLE 30

| Ex- | | | ES-MS |
|---|---|---|---|
| ample | Chemical Name | Structure | m/z |
| 42^{a} | (1S,2S)-N-((R)-1-((2-Methyl- 1-(5′-(trifluoromethyl)- 1H,2′H-[3,3′-b] pyrazol]-2′- yl)propan-2-yl)amino)-3-(1- methyl-1H-pyrazol-4-yl)-1- oxopropan-2-yl)-2-(4-(2- methyl-2H-pyrazolo[3,4- b]pyridin-5- yl)phenyl)cyclopropane-1- carboxamide | 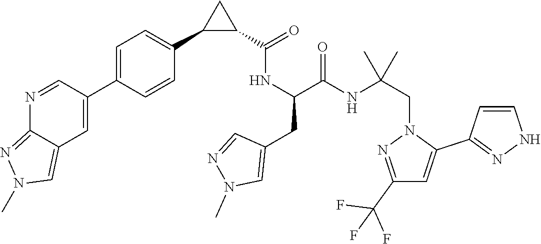 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cc[nH]n2)cn1] | 700.2 (M + H) |
| 43^{b} | (1S,2S)-N-((R)-1-((1-(5-(2H- 1,2,3-Triazol-4-yl)-3- (trifluoromethyl)-1H-pyrazol- 1-yl)-2-methylpropan-2- yl)amino)-3-(1- (difluoromethyl)-1H-pyrazol- 4-yl)-1-oxopropan-2-yl)-2-(4- (2-methyl-2H-pyrazolo[3,4- b]pyridin-5- yl)phenyl)cyclopropane-1- carboxamide | 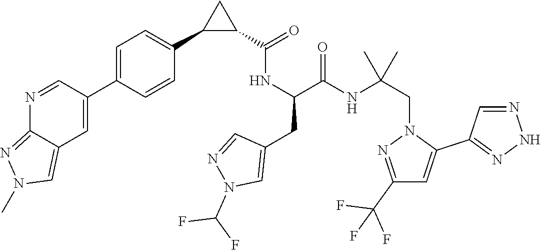 [SMILES: Cn1cc2cc(-c3ccc([C@H]4C[C@@H]4C(=O)N[C@H](Cc4cnn(C(F)F)c4)C(=O)NC(C)(C)Cn4nc(C(F)(F)F)cc4-c4cn[nH]n4)cc3)cnc2n1] | 737.2 (M + H) |
| 44^{c} | (1S,2S)-N-((R)-1-((1-(3- (Difluoromethyl)-5-(2H-1,2,3- triazol-4-yl)-1H-pyrazol-1-yl)- 2-methylpropan-2-yl)amino)- 3-(1-methyl-1H-pyrazol-4-yl)- 1-oxopropan-2-yl)-2-(4-(2- methyl-2H-pyrazolo[3,4- b]pyridin-5- yl)phenyl)cyclopropane-1- carboxamide | 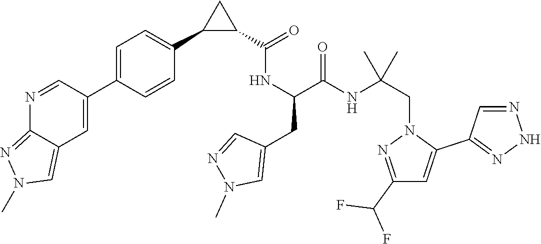 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)F)cc2-c2cn[nH]n2)cn1] | 683.2 (M + H) |

^{a}Purified by reversed phase prep-HPLC (Xtimate ® C18, 40 x 200 mm, 7 μm) eluting with 38-78% ACN in water (with 0.1% FA);
^{b}20-60%; and
^{c}6-46%.

### Example 45

### 4,4-Difluoro-N-(4-((1S,2S)-2-(((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl) carbamoyl)cyclopropyl)phenyl)cyclohexane-1-carboxamide

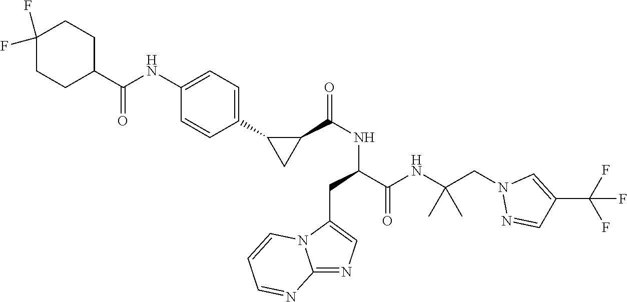 [SMILES: CC(C)(Cn1cc(C(F)(F)F)cn1)NC(=O)[C@@H](Cc1cnc2ncccn12)NC(=O)[C@H]1C[C@@H]1c1ccc(NC(=O)C2CCC(F)(F)CC2)cc1]

Under N₂, (1S,2S)-2-(4-(4,4-difluorocyclohexane-1-carboxamido)phenyl)cyclopropane-1-carboxylic acid (100 mg, 309 μmol) was treated with THF (2.06 mL), 50% propanephosphonic acid anhydride in EtOAc (228 μL, 387 μmol) and DIEA (323 μL, 1.86 mmol). After stirring at RT for 3 min, (R)-2-amino-3-(imidazo[1,2-a]pyrimidin-3-yl)-N-(2-methyl-1-(4-(trifluoromethyl)-1H-pyrazol-1-yl)propan-2-yl)propanamide hydrochloride (162 mg, 346 μmol) was added as a solution in DMF (2.5 mL) and the resulting mixture was stirred at RT for 16 h. The reaction mixture was diluted with a mixture of water (12.5 mL) and sat. aq. NaHCO₃ (12.5 mL) then EtOAc (25 mL). The layers were separated, and the aq. layer was extracted with EtOAc (3×20 mL). The organic layers were combined, concentrated under reduced pressure, and purified by reversed phase prep-HPLC (XSelect® CSH C18, 30×150 mm, 5 μm) using a gradient of 5-95% ACN in water (with 10 mM NH₄OAc, pH=6.8) over 20 min to obtain the title compound (98.4 mg, 45%) as a white powder. ES-MS m/z 701.2 (M+H).

The following compounds in Table 31 were prepared as described in Example 45 using the appropriate carboxylic acid or carboxylate salt and the appropriate amine or amine hydrochloride salt. Reactants can be added in differing equivalency. Reaction times and methods of work up may vary. Various methods can be used to purify the compounds (such as-high, neutral, or low pH prep-HPLC) which would be apparent to one skilled in the art.

TABLE 31

| Ex- | | | |
|---|---|---|---|
| am- | | | ES-MS |
| ple | Chemical Name | Structure | m/z |
| 46 | 3,3-Difluoro- N-(4-((1S,2S)-2- (((R)-3-(imidazo[1,2- a]pyrimidin- 3-yl)-1-((2- methyl-1-(4- (trifluoromethyl)- 1H-pyrazol-1- yl)propan-2- yl)amino)-1- oxopropan-2- yl)carbamoyl) cyclopropyl) phenyl) cyclobutane-1- carboxamide | 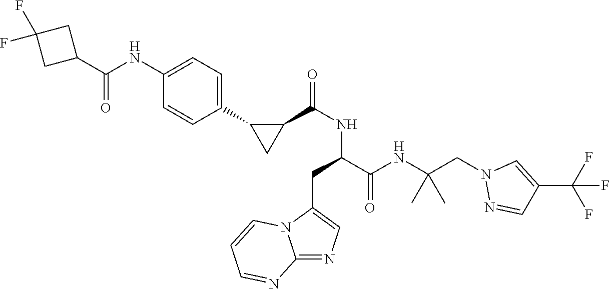 [SMILES: CC(C)(Cn1cc(C(F)(F)F)cn1)NC(=O)[C@@H](Cc1cnc2ncccn12)NC(=O)[C@H]1C[C@@H]1c1ccc(NC(=O)C2CC(F)(F)C2)cc1] | 673.2 (M + H) |
| 47 | (1S,2S)-N-((R)-3- (Imidazo[1,2-a] pyrimidin-3- yl)-1-((2-methyl-1-(4- (trifluoromethyl)- 1H-pyrazol- 1-yl)propan- 2-yl)amino)-1- oxopropan-2-yl)-2-(4- (imidazo[1,2- a]pyrimidin-6- yl)phenyl) cyclopropane-1- carboxamide | 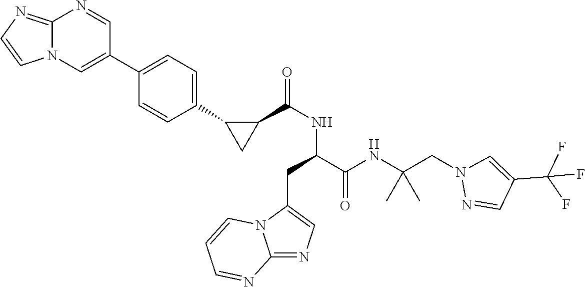 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nccn3c2)cc1] | 657.4 (M + H) |
| 48 | 3,3-Difluoro- N-(4-((1S,2S)-2- (((R)-1-((1-(4- fluorophenyl)- 2-methylpropan- 2-yl)amino)- 3-(imidazo[1,2-a] pyrimidin-3- yl)-1-oxopropan-2- yl)carbamoyl) cyclopropyl) phenyl)cyclobutane-1- carboxamide | 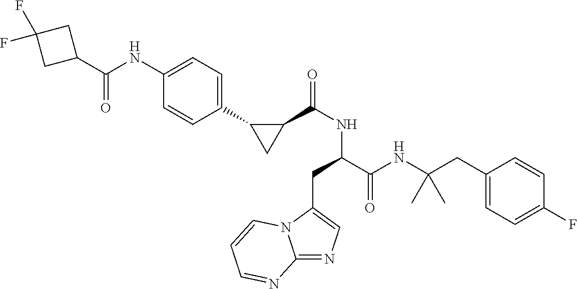 [SMILES: CC(C)(Cc1ccc(F)cc1)NC(=O)[C@@H](Cc1cnc2ncccn12)NC(=O)[C@H]1C[C@@H]1c1ccc(NC(=O)C2CC(F)(F)C2)cc1] | 633.4 (M + H) |
| 49 | 4,4-Difluoro- N-(4-((1S,2S)-2- (((R)-1-((1- (4-fluorophenyl)- 2-methylpropan- 2-yl)amino)- 3-(imidazo[1,2-a] pyrimidin-3- yl)-1-oxopropan-2- yl)carbamoyl) cyclopropyl) phenyl)cyclohexane-1- carboxamide | 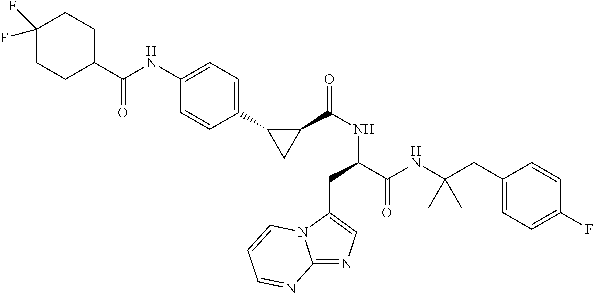 [SMILES: CC(C)(Cc1ccc(F)cc1)NC(=O)[C@@H](Cc1cnc2ncccn12)NC(=O)[C@H]1C[C@@H]1c1ccc(NC(=O)C2CCC(F)(F)CC2)cc1] | 661.4 (M + H) |
| 50^{a} | (trans)-N-((R)-1-((1-(4- Fluorophenyl)-2- methylpropan-2- yl)amino)-3- (1-methyl-1H- pyrazol-4-yl)-1- oxopropan-2- yl)-2-(4-(3-(4- fluorophenyl)ureido)-3- methylphenyl) cyclopropane- 1-carboxamide, Isomer 2 | 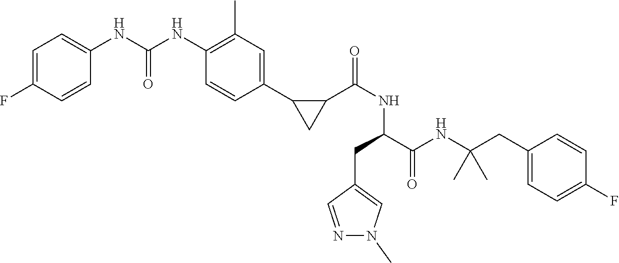 [SMILES: Cc1cc(C2CC2C(=O)N[C@H](Cc2cnn(C)c2)C(=O)NC(C)(C)Cc2ccc(F)cc2)ccc1NC(=O)Nc1ccc(F)cc1] | 629.0 (M + H) |

^{a}Second-eluting isomer when purified by reversed phase prep-HPLC (XSelect ® CSH C18, 30 × 150 mm, 5 μm, 40 mL/min, RT) eluting with 5-95% ACN in water (with 10 mM NH₄OAc, pH = 6.8) over 38.5 min.

### Example 51

### (1S,2S)-N—((R)-1-((1-(5-Cyclobutyl-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

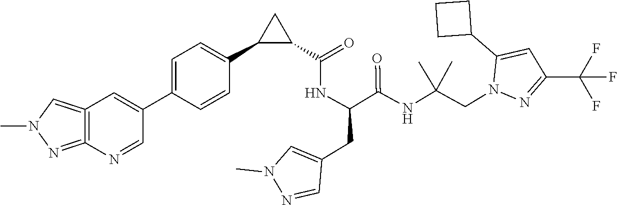

A solution of (R)-3-(1-methyl-1H-pyrazol-4-yl)-2-((1S,2S)-2-(4-(2-methyl-2H-pyrazolo[3,4-b]pyridin-5-yl)phenyl)cyclopropane-1-carboxamido)propanoic acid (24 mg, 54 μmol) and 1-(5-cyclobutyl-3-(trifluoromethyl)-1H-pyrazol-1-yl)-2-methylpropan-2-amine HCl (16 mg, 54 μmol) in THF (1.3 mL) under N₂ was treated with TEA (30 μL, 210 μmol) and cooled to 0° C. Diethyl (4-oxo-1,2,3-benzotriazin-3-yl)phosphate (19 mg, 64 μmol) was added in one portion, and the resulting mixture was stirred at 23° C. for 15 h. The reaction mixture was diluted with EtOAc (4 mL) and washed with water (2×2 mL). The organic phase was dried over MgSO₄ and concentrated under reduced pressure. The residue was purified by reversed phase prep-HPLC (Kinetex® EVO C18, 30×100 mm, 5 μm, 45° C., 85 mL/min) using a gradient of 33-67% ACN in water (with 0.1% FA) over 6 min to obtain the title compound (15 mg, 41%) as a white solid. ES-MS m/z 688.4 (M+H).

### Example 52

### (1S,2S)-N—((R)-1-((2-Methyl-1-(5-(1-methyl-2-oxo-1,2-dihydropyridin-4-yl)-3-(trifluoromethyl)-1H-pyrazol-1-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

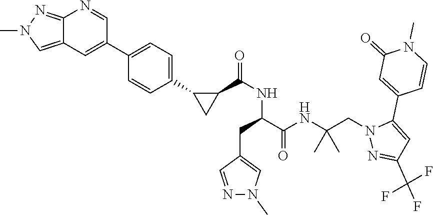 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2ccn(C)c(=O)c2)cn1]

A mixture of (1S,2S)-N—((R)-1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide (50.0 mg, 70.2 μmol), 1-methyl-4-(4,4,5,5-tetramethyl-1,3,2-dioxaborolan-2-yl)pyridin-2(1H)-one (33.0 mg, 140 μmol), Pd(dppf)Cl₂·CH₂Cl₂ (11.5 mg, 14.0 μmol) and K₂CO₃ (29.1 mg, 211 μmol) in 3:1 1,4-dioxane water (1.4 mL) was sparged with argon for 5 min, and the resulting mixture was stirred at 95° C. overnight. The reaction mixture was partitioned between sat. aq. NaHCO₃ (2 mL) and EtOAc (2 mL), and the aq. layer was extracted with EtOAc (3× 2 mL). The organic layers were combined, washed with sat. aq. NaCl (5 mL), stirred with SiliaMetS® metal scavenger resin, then dried over Na₂SO₄ and filtered over a pad of diatomaceous earth. The filtrate was concentrated under reduced pressure, and the residue was purified by reversed phase prep-HPLC (XSelect® CSH C18, 30×150 mm, 5 μm, 40 mL/min) eluting with 5-95% ACN in water (with 10 mM NH₄OAc, pH=6.8) over 20 min to obtain the title compound (32.9 mg, 63%) as a white solid. ES-MS m z 741.4 (M+H).

The compounds in the following table were prepared as described in Example 52 using the appropriate boron ester or boronic acid and the appropriate aryl bromide coupling partners. Reactants can be added in different orders or in differing equivalency. Pd(dppf)Cl₂ is a suitable catalyst and Cs₂CO₃ is a suitable base. Reaction times (1 h to overnight) and temperatures (80-95° C.) may vary, and differing methods can be used to work up or purify the compounds (normal phase, high or low pH prep-HPLC). Such variances would be apparent to one skilled in the art.

TABLE 32

| Ex- | | | |
|---|---|---|---|
| am- | | | ES-MS |
| ple | Chemical Name | Structure | m/z |
| 53^{a,d} | (1S,2S)-N-((R)- 1-((2-Methyl- 1-(1-((RS)- tetrahydrofuran- 3-yl)-5′- (trifluoromethyl)- 1H,2′H-[3,3′- bipyrazol]-2′- yl)propan-2- yl)amino)-3-(1- methyl-1H- pyrazol-4-yl)-1- oxopropan- 2-yl)-2-(4-(2- methyl-2H- pyrazolo[3,4- b]pyridin-5- yl)phenyl) cyclopropane-1- | 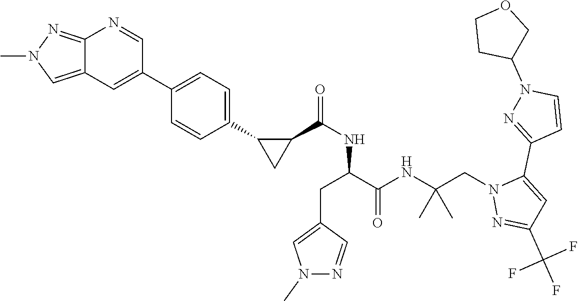 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2ccn(C3CCOC3)n2)cn1] | 770.4 (M + H) |
| | carboxamide, | | |
| | mixture of 2 | | |
| | diastereomers | | |
| 54^{a,d} | (1S,2S)-N-((R)- 1-((2-Methyl- 1-(1′-(oxetan-3- yl)-5- (trifluoromethyl)- 1′H,2H- [3,4′-bipyrazol]- 2-yl)propan- 2-yl)amino)-3- (1-methyl-1H- pyrazol-4-yl)- 1-oxopropan-2- yl)-2-(4-(2-methyl- 2H-pyrazolo[3,4- b]pyridin-5- yl)phenyl) cyclopropane-1- carboxamide | 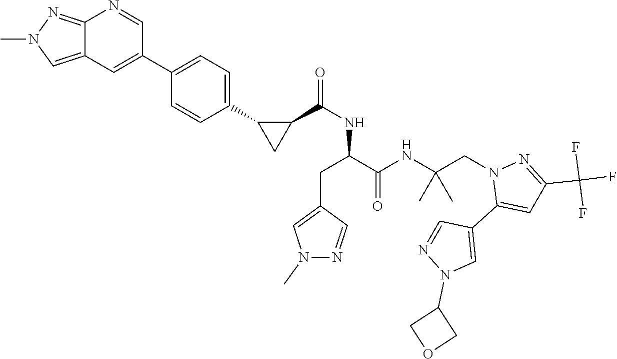 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cnn(C3COC3)c2)cn1] | 756.4 (M + H) |
| 55^{a,d} | (1S,2S)-N-((R)- 1-((2-Methyl- 1-(1′-(oxetan- 3-ylmethyl)-5- (trifluoromethyl)- 1′H,2H- [3,4′-bipyrazol]- 2-yl)propan- 2-yl)amino)-3- (1-methyl-1H- pyrazol-4-yl)- 1-oxopropan-2- yl)-2-(4-(2- methyl-2H- pyrazolo[3,4- b]pyridin-5- yl)phenyl) cyclopropane-1- carboxamide | 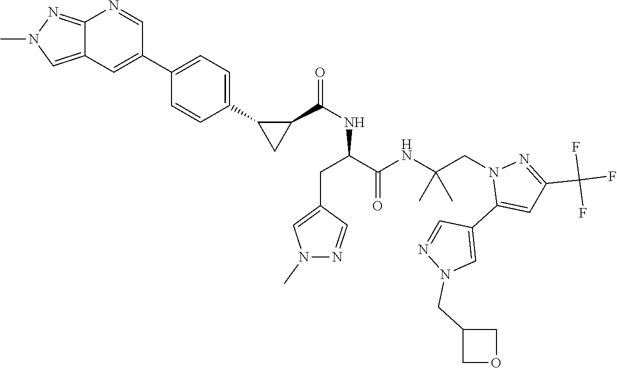 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cnn(CC3COC3)c2)cn1] | 770.4 (M + H) |
| 56^{e} | tert-Butyl 3- (1-(2-methyl-2- ((R)-3-(1-methyl- 1H-pyrazol- 4-yl)-2- ((1S,2S)-2-(4-(2- methyl-2H- pyrazolo[3,4- b]pyridin-5-yl) phenyl) cyclopropane- 1-carboxamido) propanamido) propyl)-3- (trifluoromethyl)-1H- pyrazol-5-yl)-6,7- | 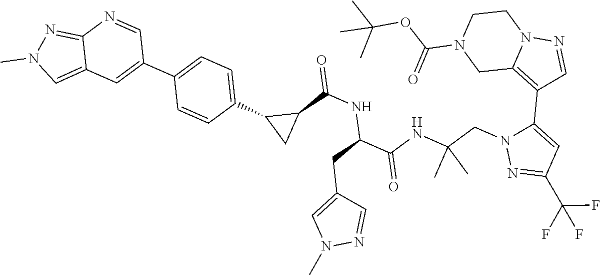 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cnn3c2CN(C(=O)OC(C)(C)C)CC3)cn1] | 855.4 (M + H) |
| | dihydropyrazolo[1,5- | | |
| | a]pyrazine-5(4H)- | | |
| | carboxylate | | |
| 57^{e} | (1S,2S)-N-((R)- 1-((2-Methyl- 1-(5-(4-oxo-3,4- dihydropyrrolo[2,1- f][1,2,4]triazin- 6-yl)-3- (trifluoromethyl)- 1H-pyrazol- 1-yl)propan-2- yl)amino)-3-(1- methyl-1H- pyrazol-4-yl)-1- oxopropan-2-yl)-2- (4-(2-methyl-2H- pyrazolo[3,4- b]pyridin-5- yl)phenyl) cyclopropane- 1-carboxamide | 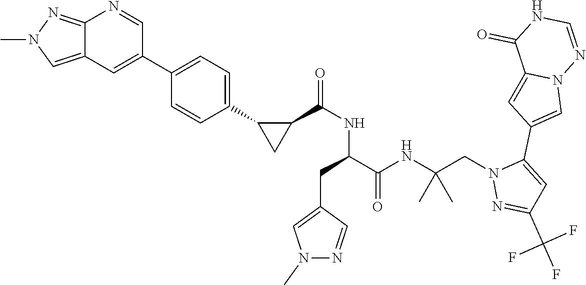 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cc3c(=O)[nH]cnn3c2)cn1] | 767.4 (M + H) |
| 58 | (1S,2S)-N-((R)- 1-((2-Methyl-1-(5- (1-methyl-2-oxo-1,2- dihydropyridin- 3-yl)-3- (trifluoromethyl)- 1H-pyrazol- 1-yl)propan-2-yl) amino)-3-(1- methyl-1H- pyrazol-4-yl)-1- oxopropan-2-yl)-2- (4-(2-methyl-2H- pyrazolo[3,4- b]pyridin-5- yl)phenyl) cyclopropane- 1-carboxamide | 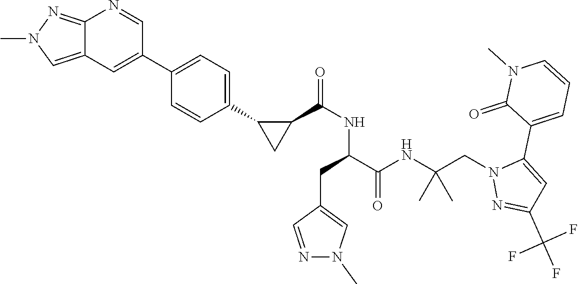 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cccn(C)c2=O)cn1] | 741.4 (M + H) |
| 59^{c} | (1S,2S)-N-((R)- 1-((2-Methyl- 1-(5-(6-oxo-1,6- dihydropyridin-3-yl)- 3-(trifluoromethyl)- 1H-pyrazol- 1-yl)propan-2- yl)amino)-3-(1- methyl-1H- pyrazol-4-yl)-1- oxopropan-2-yl)-2- (4-(2-methyl-2H- pyrazolo[3,4- b]pyridin-5- yl)phenyl) cyclopropane- 1-carboxamide | 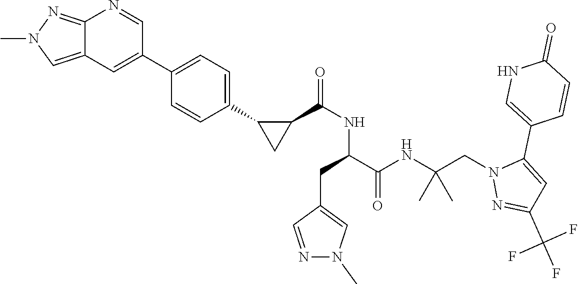 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2ccc(=O)[nH]c2)cn1] | 727.4 (M + H) |
| 60^{a,g} | (trans)-N-((R)- 1-((2-Methyl- 1-(5-(1-methyl- 6-oxo-1,6- dihydropyridin-3-yl)- 3-(trifluoromethyl)- 1H-pyrazol- 1-yl)propan-2-yl) amino)-3-(1- methyl-1H- pyrazol-4-yl)-1- oxopropan-2- yl)-2-(5- (2-methyl-2H- pyrazolo[3,4- b]pyridin-5- yl)thiophen-2- yl)cyclopropane-1- carboxamide, Isomer 2 | 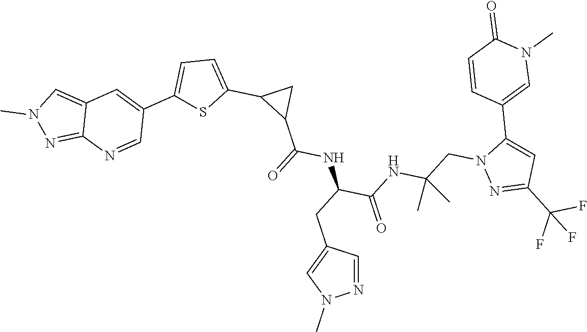 [SMILES: Cn1cc(C[C@@H](NC(=O)C2CC2c2ccc(-c3cnc4nn(C)cc4c3)s2)C(=O)NC(C)(C)Cn2nc(C(F)(F)F)cc2-c2ccc(=O)n(C)c2)cn1] | 747.4 (M + H) |
| 61 | (1S,2S)-2- (2-Fluoro-4-(2- methyl-2H-pyrazolo [3,4-b]pyridin-5- yl)phenyl)-N- methyl-N-((R)- 1-((2-methyl-1- (5-(1-methyl-6- oxo-1,6- dihydropyridin- 3-yl)-3- (trifluoromethyl)- 1H-pyrazol- 1-yl)propan-2-yl) amino)-3-(1- methyl-1H-pyrazol-4-yl)- 1-oxopropan-2-yl) cyclopropane- 1-carboxamide | 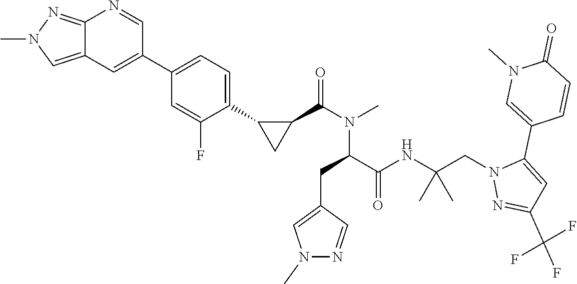 [SMILES: CN(C(=O)[C@H]1C[C@@H]1c1ccc(-c2cnc3nn(C)cc3c2)cc1F)[C@H](Cc1cnn(C)c1)C(=O)NC(C)(C)Cn1nc(C(F)(F)F)cc1-c1ccc(=O)n(C)c1] | 773.2 (M + H) |
| 62^{e} | (1S,2S)-N-((R)-3- (imidazo[1,2-a] pyrimidin-3-yl)- 1-((2-methyl-1-(5-(1- methyl-6-oxo-1,6- dihydropyridin- 3-yl)-3- (trifluoromethyl)- 1H-pyrazol- 1-yl)propan-2-yl) amino)-1- oxopropan-2-yl)-2- (4-(2-methyl-2H- pyrazolo[3,4- b]pyridin-5- yl)phenyl) cyclopropane- 1-carboxamide | 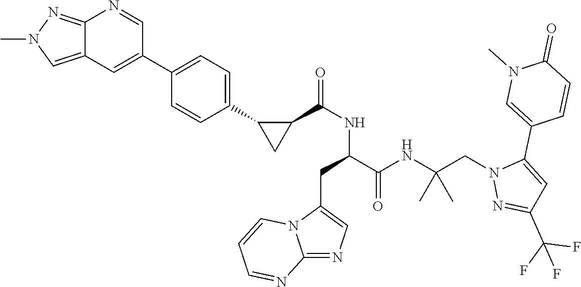 [SMILES: Cn1cc2cc(-c3ccc([C@H]4C[C@@H]4C(=O)N[C@H](Cc4cnc5ncccn45)C(=O)NC(C)(C)Cn4nc(C(F)(F)F)cc4-c4ccc(=O)n(C)c4)cc3)cnc2n1] | 778.2 (M + H) |
| 63^{b,f} | (R)-3-(1- (Difluoromethyl)- 1H-pyrazol-4-yl)- N-(2- methyl-1-(5-(1- methyl-6-oxo- 1,6-dihydropyridin- 3-yl)-3- (trifluoromethyl)- 1H-pyrazol- 1-yl)propan-2-yl)- 2-((1R,3R)- 1-(4-(2-methyl-2H- pyrazolo[3,4- b]pyridin-5- yl)phenyl)-4-oxo-5- azaspiro[2.4] | 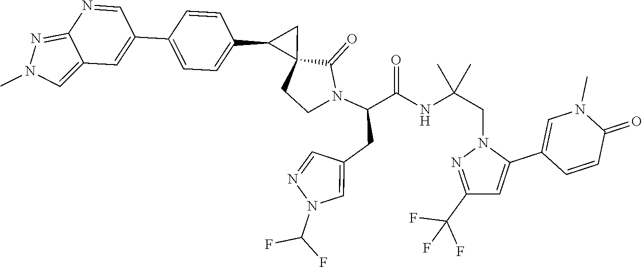 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccc(=O)n(C)c4)C5=O)cc3)cnc2n1] | 801.2 (M + H) |
| | heptan-5- | | |
| | yl)propanamide | | |

^{a}Catalyst: Pd(dppf)Cl₂, base: Cs₂CO₃, reaction temperature/time: 90° C. for 1 h.
^{b}Reaction temperature/time: 80° C. for 1.5 h.
^{c}Reaction temperature/time: 95° C. for 6 h.
^{d}Purified by silica gel chromatography using a gradient of 0-100% 3:1 EtOAc/EtOH in heptane.
^{e}Eluted with 5-95% ACN in water (with 0.1% FA)
^{f}Eluted with 25-50% ACN/10 mM aq. NH₄HCO₃, pH = 10 (with 5% MeOH).
^{g}Eluted with 20-35% ACN/10 mM aq. NH₄HCO₃, pH = 10 (with 5% MeOH) over 13 min, held at 35% for 6 min.

### Example 64

### (1S,2S)-N—((R)-1-((2-Methyl-1-(5-(6-oxo-1,6-dihydropyridin-2-yl)-3-(trifluoromethyl)-1H-pyrazol-1-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

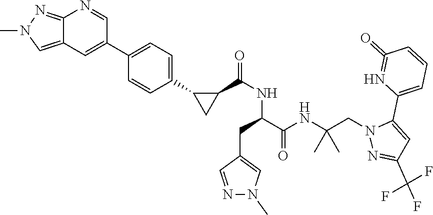

To a mixture of (1S,2S)-N—((R)-1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide (20 mg, 28 μmol), 6-(4,4,5,5-tetramethyl-1,3,2-dioxaborolan-2-yl)pyridin-2(1H)-one (12 mg, 56 μmol), mesylate[(di(1-adamantyl)-n-butylphosphine)-2-(2′-amino-1,1′-biphenyl)]palladium (II) or cataCXium® A Pd G3 (CAS #1651823-59-4, 2.0 mg, 2.8 μmol) and CsF (17 mg, 0.11 mmol) under N₂ was added n-BuOH (1.3 mL) and water (0.13 mL). The resulting mixture was sparged with N₂ for 5 min, then stirred at 95° C. overnight. A second reaction was performed on the same scale using the same method except the catalyst was Pd (amphos)Cl₂ (CAS #887919-35-9, 2.0 mg, 2.8 μmol). The two reaction mixtures were combined and partitioned between EtOAc (10 mL) and sat. aq. NaCl (10 mL), and the organic layer was washed with sat. aq. NaCl (2×5 mL) then concentrated under a stream of N₂. The residue was purified by reversed phase prep-HPLC (XSelect® CSH C18, 30×150 mm, 5 μm, 40 mL/min) eluting with 5-95% ACN in water (with 10 mM NH₄OAc, pH=6.8) over 42 min to obtain the title compound (2.4 mg, 6%) as a white solid. ES-MS m/z 727.4 (M+H).

### Example 65

### (1S,2S)-N—((R)-1-((1-(5-(2-(Methoxymethyl)-2H-1,2,3-triazol-4-yl)-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

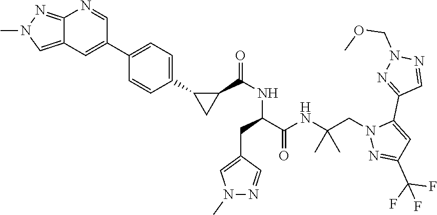 [SMILES: COCn1ncc(-c2cc(C(F)(F)F)nn2CC(C)(C)NC(=O)[C@@H](Cc2cnn(C)c2)NC(=O)[C@H]2C[C@@H]2c2ccc(-c3cnc4nn(C)cc4c3)cc2)n1]

A mixture of (1S,2S)-N—((R)-1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide (210.1 mg, 1 Eq, 294.8 μmol), 2-(methoxymethyl)-4-(4,4,5,5-tetramethyl-1,3,2-dioxaborolan-2-yl)-2H-1,2,3-triazole (176 mg, 737 μmol) and K₃PO₄ (188 mg, 885 μmol) in DME (6 mL) was sparged with argon for 2 min. Pd(PPh₃)₄ (68.14 mg, 58.97 μmol) was added, and argon degassing was continued for 3 min. The resulting mixture was stirred at 95° C. for 5.5 h. The reaction mixture was cooled to RT and partitioned between water (15 mL) and EtOAc (30 mL). The aq. layer was extracted with EtOAc (3×25 mL). The organic layers were combined, washed with water (15 mL) then sat. aq. NaCl (15 mL), dried over Na₂SO₄, filtered through a pad of diatomaceous earth, and concentrated under reduced pressure. The residue was purified by reversed phase prep-HPLC (XSelect® CSH C18, 30×150 mm, 5 μm, 40 mL/min) eluting with 5-95% ACN in water (with 10 mM NH₄OAc, pH=6.8) over 20 min to obtain the title compound (122.3 g, 55%) as a white solid. ES-MS m z 745.4 (M+H).

### Example 66

### (1S,2S)-N—((R)-1-((2-Methyl-1-(5-(2-methyl-2H-1,2,3-triazol-4-yl)-3-(trifluoromethyl)-1H-pyrazol-1-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

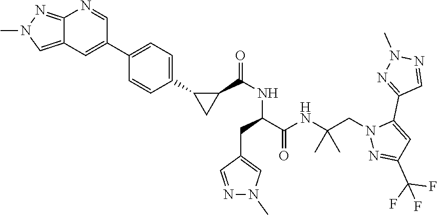 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cnn(C)n2)cn1]

A mixture of (1S,2S)-N—((R)-1-((1-(5-bromo-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide (75.0 mg, 105 μmol), 2-(2-methyl-2H-1,2,3-triazol-4-yl)-1,3,6,2-dioxazaborocane (61.9 mg, 316 μmol), Pd(dppf)Cl₂·CH₂Cl₂ (8.60 mg, 11 μmol), Cs₂CO₃ (137 mg, 421 μmol), and copper (I) chloride (15.6 mg, 158 μmol) was purged with N₂, and DMF (1.0 mL) was added. The reaction mixture was sparged with N₂ for 5 min and then stirred at 100° C. for 3 h before cooling to RT and combining with a second reaction run on 70 μmol scale. The combined mixture was diluted with sat. aq. NaHCO₃ (30 mL), sat. aq. NaCl (10 mL), and EtOAc (15 mL). The layers were separated, and the aq. layer was extracted with EtOAc (4×20 mL). The organic layers were combined, stirred with SiliaMetS® metal scavenger resin (5 g) for 20 min and filtered, rinsing with additional EtOAc (2× 15 mL). The filtrate was concentrated under reduced pressure, and the residue was purified by reversed phase prep-HPLC (XSelect® CSH C18, 30×150 mm, 5 μm, 40 mL/min) eluting with 5-95% ACN in water (with 0.1% FA) over 20 min to obtain the title compound (25.0 mg, 20%) as a white powder. ES-MS m/z 715.4 (M+H).

### Example 67

### (1S,2S)-N—((R)-1-((1-(1-(((trans)-3-fluorocyclobutyl)methyl)-5′-(trifluoromethyl)-1H,2′H-[3,3′-bipyrazol]-2′-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

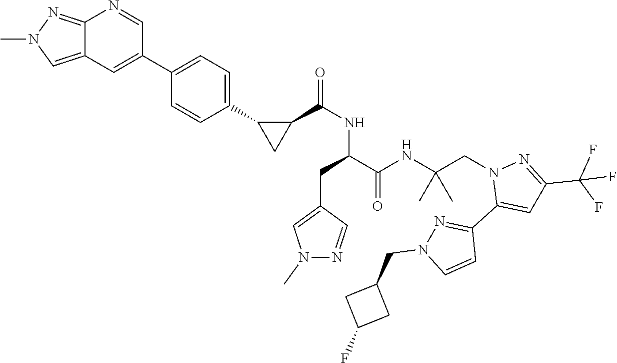 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2ccn(C[C@H]3C[C@H](F)C3)n2)cn1]

The title compound was prepared as described in Example 66 using 1-(((trans)-3-fluorocyclobutyl)methyl)-4-(4,4,5,5-tetramethyl-1,3,2-dioxaborolan-2-yl)-1H-pyrazole with purification by reversed phase prep-HPLC (XSelect® CSH C18, 30×150 mm, 5 μm, 60 mL/min) eluting with 30-53% ACN in 10 mM aq. NH₄HCO₃, pH=10 (with 5% MeOH) over 25 min, followed by a second purification by reversed phase prep-HPLC (XSelect® CSH C18, 30×150 mm, 5 μm, 40 mL/min) eluting with 5-95% ACN in water (with 0.1% FA) over 20 min. ES-MS m/z 786.4 (M+H).

### Example 68

### (1S,2S)-N—((R)-1-((1-(5-(2H-1,2,3-Triazol-4-yl)-3-(trifluoromethyl)-1H-pyrazol-1-yl)-2-methylpropan-2-yl)amino)-3-(imidazo[1,2-a]pyrimidin-3-yl)-1-oxopropan-2-yl)-2-(4-(2-(trifluoromethyl) imidazo[1,2-a]pyrimidin-6-yl)phenyl)cyclopropane-1-carboxamide

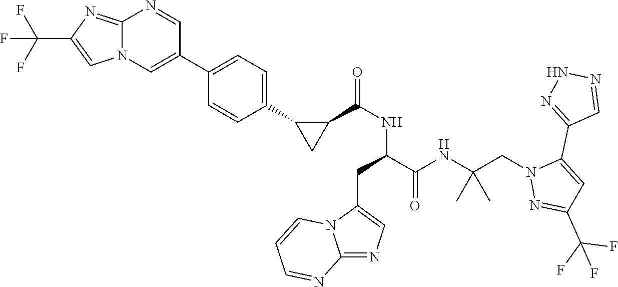 [SMILES: CC(C)(Cn1nc(C(F)(F)F)cc1-c1cn[nH]n1)NC(=O)[C@@H](Cc1cnc2ncccn12)NC(=O)[C@H]1C[C@@H]1c1ccc(-c2cnc3nc(C(F)(F)F)cn3c2)cc1]

A mixture of (1S,2S)-N—((R)-1-((1-(5-bromo-3-(trifluoromethyl)-1H-pyrazol-1-yl)-2-methylpropan-2-yl)amino)-3-(imidazo[1,2-a]pyrimidin-3-yl)-1-oxopropan-2-yl)-2-(4-(2-(trifluoromethyl) imidazo[1,2-a]pyrimidin-6-yl)phenyl)cyclopropane-1-carboxamide (241 mg, 300 μmol), 2-(tetrahydro-2H-pyran-2-yl)-4-(4,4,5,5-tetramethyl-1,3,2-dioxaborolan-2-yl)-2H-1,2,3-triazole (209 mg, 750 μmol), Pd(dppf)Cl₂·CH₂Cl₂ (24.5 mg, 30.0 μmol), Cs₂CO₃ (244 mg, 750 μmol), and copper (I) chloride (45.0 mg, 450 μmol) in a reaction vial was purged with N₂, and DMF (4.0 mL) was added. The resulting mixture was sparged with N₂ for 5 min then stirred at 100° C. for 1.5 h. The reaction mixture was cooled to RT, diluted with water (20 mL) and 4 M HCl in 1,4-dioxane (10 mL). The mixture was stirred at 60° C. for 30 min then cooled to RT, treated with SiliaMetS® metal scavenger resin (2 g) then stirred for 45 min and filtered over diatomaceous earth, rinsing with EtOH (2×15 mL). The filtrate was concentrated under reduced pressure, and the residue was purified by reversed phase prep-HPLC (XSelect® CSH C18, 30×150 mm, 5 μm, 40 mL/min), eluting with a gradient of 5-95% ACN in water (with 10 mM NH₄OAc, pH-6.8) over 20 min to obtain the title compound (67.8 mg, 29%) as a white powder. ES-MS m/z 792.2 (M+H).

The compounds in the following table were prepared as described in Example 68 using the appropriate aryl bromide coupling partners. Various methods were used to purify the compounds, which would be apparent to one skilled in the art.

TABLE 33

| Ex- | | | ES-MS |
|---|---|---|---|
| ample | Chemical Name | Structure | m/z |
| 69 | (1S,2S)-N-((R)- 1-((1-(5-(2H- 1,2,3-Triazol-4-yl)-3- (trifluoromethyl)- 1H-pyrazol- 1-yl)-2-methylpropan-2- yl)amino)-3-(imidazo[1,2- a]pyrimidin-3-yl)-1- oxopropan-2-yl)-2-(4-(2- methyl-2H-pyrazolo [3,4-b]pyridin-5- yl)phenyl)cyclopropane- 1-carboxamide | 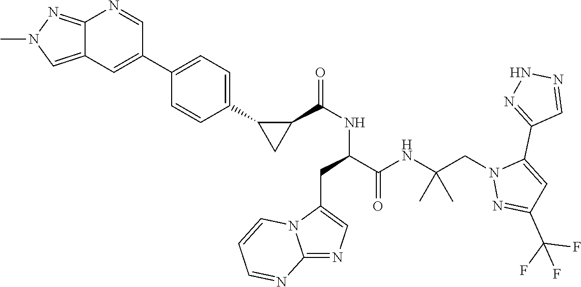 [SMILES: Cn1cc2cc(-c3ccc([C@H]4C[C@@H]4C(=O)N[C@H](Cc4cnc5ncccn45)C(=O)NC(C)(C)Cn4nc(C(F)(F)F)cc4-c4cn[nH]n4)cc3)cnc2n1] | 738.2 (M + H) |
| 70^{a} | (1S,2S)-N-((R)- 1-((1-(5-(2H- 1,2,3-Triazol-4-yl)-3- (trifluoromethyl)- 1H-pyrazol- 1-yl)-2-methylpropan-2- yl)amino)-3-(1-methyl-1H- pyrazol-4-yl)-1-oxopropan- 2-yl)-N-methyl- 2-(4-(2-methyl- 2H-pyrazolo [3,4-b]pyridin-5- yl)phenyl)cyclopropane- 1-carboxamide | 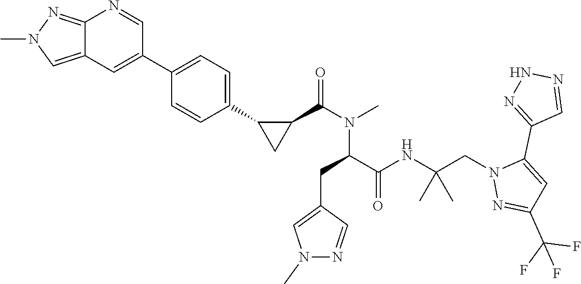 [SMILES: CN(C(=O)[C@H]1C[C@@H]1c1ccc(-c2cnc3nn(C)cc3c2)cc1)[C@H](Cc1cnn(C)c1)C(=O)NC(C)(C)Cn1nc(C(F)(F)F)cc1-c1cn[nH]n1] | 715.4 (M + H) |

^{a}Purified by prep-HPLC (XSelect ® CSH C18, 30 × 150 mm, 5 μm, 40 mL/min) using a gradient of 5-95% ACN in water (with 0.1% FA) over 20 min.

### Example 71

### (trans)-2-(2-Fluoro-4-(2-methyl-2H-pyrazolo[3,4-b]pyridin-5-yl)phenyl)-N—((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl)cyclopropane-1-carboxamide, Isomer 2

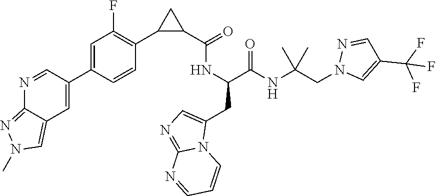 [SMILES: Cn1cc2cc(-c3ccc(C4CC4C(=O)N[C@H](Cc4cnc5ncccn45)C(=O)NC(C)(C)Cn4cc(C(F)(F)F)cn4)c(F)c3)cnc2n1]

To a mixture of (2-methyl-2H-pyrazolo[3,4-b]pyridin-5-yl) boronic acid (120 mg, 678 μmol) and (trans)-2-(4-bromo-2-fluorophenyl)-N—((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl)cyclopropane-1-carboxamide (mixture of 2 diastereomers, 210 mg, 330 μmol), Pd(dppf)Cl₂·CH₂Cl₂ (35 mg, 48 μmol), and Cs₂CO₃ (350 mg, 1.07 mmol) was added 3:1 1,4-dioxane/water (5 mL), and the resulting mixture was stirred at RT for 5 min, then at 90° C. for 1 h. The reaction mixture was cooled to RT, diluted with 1,4-dioxane (6 mL) and EtOAc (6 mL), and stirred. The layers were separated, and the aq. layer was acidified to pH3 with 1N aq. HCl (˜0.7 mL) before extracting with EtOAc (3×30 mL). All organic layers were combined, dried over Na₂SO₄, and concentrated under reduced pressure. The residue was purified by reversed phase prep-HPLC (XSelect® CSH C18, 30×150 mm, 5 μm, 60 mL/min, 3 injections) eluting with 15-35% ACN in 10 mM aq. NH₄HCO₃, pH=10 (with 5% MeOH) over 20 min with a 5 min hold at 35% to obtain the second eluting isomer as the title compound (55 mg, 23%). ES-MS m/z 689.2 (M+H).

### Example 72

### (trans)-2-(5-(2-(Difluoromethyl)-2H-pyrazolo[3,4-b]pyridin-5-yl)pyridin-2-yl)-N—((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl)cyclopropane-1-carboxamide, Isomer 2

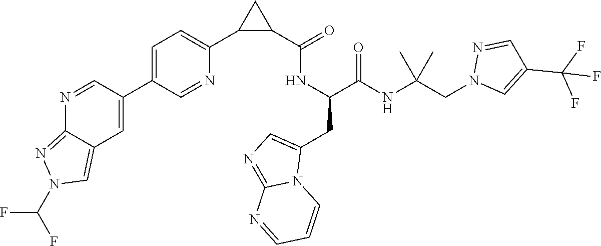 [SMILES: CC(C)(Cn1cc(C(F)(F)F)cn1)NC(=O)[C@@H](Cc1cnc2ncccn12)NC(=O)C1CC1c1ccc(-c2cnc3nn(C(F)F)cc3c2)cn1]

The title compound was prepared as described in Example 61 using (trans)-2-(5-bromopyridin-2-yl)-N—((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl)cyclopropane-1-carboxamide and 2-(difluoromethyl)-5-(4,4,5,5-tetramethyl-1,3,2-dioxaborolan-2-yl)-2H-indazole and isolated as the second eluting isomer by reversed phase prep-HPLC (XSelect® CSH C18, 30×150 mm, 5 μm, 60 mL/min) eluting with a gradient of 23-38% ACN/10 mM aq. NH₄HCO₃, pH=10 (with 5% MeOH) over 16 min. ES-MS m/z 708.2 (M+H).

### Example 73

### (1S,2S)-N—((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl)-2-(4-(2-(trifluoromethyl) imidazo[1,2-a]pyrimidin-6-yl)phenyl)cyclopropane-1-carboxamide

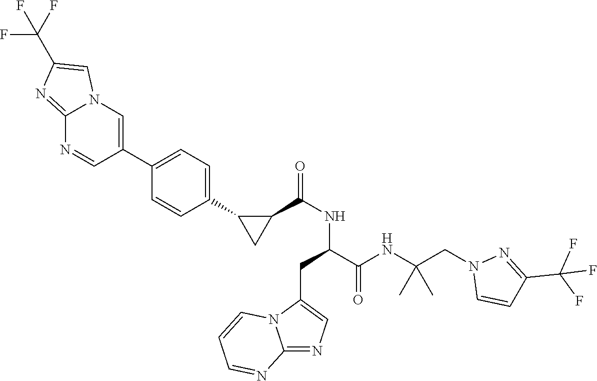 [SMILES: CC(C)(Cn1ccc(C(F)(F)F)n1)NC(=O)[C@@H](Cc1cnc2ncccn12)NC(=O)[C@H]1C[C@@H]1c1ccc(-c2cnc3nc(C(F)(F)F)cn3c2)cc1]

A mixture of (1,1′-bis(diphenylphosphino)ferrocene)palladium(II) dichloride (16.6 mg, 20.3 μmol), Cs₂CO₃ (159 mg, 487 μmol), and 6-bromo-2-(trifluoromethyl) imidazo[1,2-a]pyrimidine (89.9 mg, 338 μmol) was purged with N₂, then a solution of (1S,2S)-N—((R)-3-(imidazo[1,2-a]pyrimidin-3-yl)-1-((2-methyl-1-(4-(trifluoromethyl)-1H-pyrazol-1-yl)propan-2-yl)amino)-1-oxopropan-2-yl)-2-(4-(4,4,5,5-tetramethyl-1,3,2-dioxaborolan-2-yl)phenyl)cyclopropane-1-carboxamide (0.15 M in 1,4-dioxane, 135 μmol) and water (135 μL) was added and the mixture was sparged with N₂ for 5 min. The mixture was heated at 90° C. for 3 h, then cooled to RT and additional 6-bromo-2-(trifluoromethyl) imidazo[1,2-a]pyrimidine (50 mg) and (1,1′-bis(diphenylphosphino)ferrocene)palladium(II) dichloride (10 mg) was added. The mixture was sparged with nitrogen and heated to 90° C. for 2.5 h. The mixture was diluted with aq. NaHCO₃ (half-saturated, 10 mL) and EtOAc (8 mL) and stirred for 2 min. The mixture was extracted with EtOAc (4×8 mL), and the combined organics were stirred with SiliCycle™ SiliaMetS™ metal scavenger resin for 20 min. The mixture was filtered over diatomaceous earth and washed with EtOAc (2×5 mL). The filtrate was concentrated and purified by reverse phase HPLC using a gradient of 5 to 95% ACN in aq. NH₄OAc to give the title compound (25.2 mg, 26%) as a tan powder. ES-MS m/z 725 (M+H).

The Examples in the following table were prepared as described in Example 73 using the appropriate boronic ester and aryl bromide.

TABLE 34

| | | | ES-MS |
|---|---|---|---|
| Example | Chemical name | Structure | m/z |
| 74 | (1S,2S)-2-(4-(2-(3,3- Difluorocyclobutyl)imidazo[1,2- a]pyrimidin-6-yl)phenyl)-N-((R)- 3-(imidazo[1,2-a]pyrimidin-3- yl)-1-((2-methyl-1-(4- (trifluoromethyl)-1H-pyrazol-1- yl)propan-2-yl)amino)-1- oxopropan-2-yl)cyclopropane-1- carboxamide | 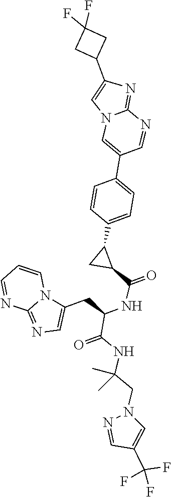 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nc(C4CC(F)(F)C4)cn3c2)cc1] | 747 (M + H) |
| 75 | (1S,2S)-N-((R)-3-(Imidazo[1,2- a]pyrimidin-3-yl)-1-((2-methyl- 1-(4-(trifluoromethyl)-1H- pyrazol-1-yl)propan-2-yl)amino)- 1-oxopropan-2-yl)-2-(4-(2- (methoxymethyl)imidazo[1,2- a]pyrimidin-6- yl)phenyl)cyclopropane-1- carboxamide | 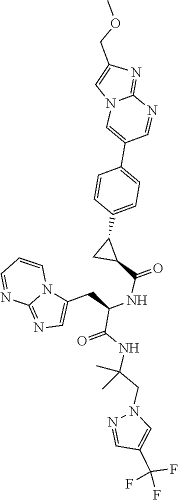 [SMILES: COCc1cn2cc(-c3ccc([C@H]4C[C@@H]4C(=O)N[C@H](Cc4cnc5ncccn45)C(=O)NC(C)(C)Cn4cc(C(F)(F)F)cn4)cc3)cnc2n1] | 701 (M + H) |
| 76 | (1S,2S)-2-(4-(2- Cyclopropylimidazo[1,2- a]pyrimidin-6-yl)phenyl)-N-((R)- 3-(imidazo[1,2-a]pyrimidin-3- yl)-1-((2-methyl-1-(4- (trifluoromethyl)-1H-pyrazol-1- yl)propan-2-yl)amino)-1- oxopropan-2-yl)cyclopropane-1- carboxamide | 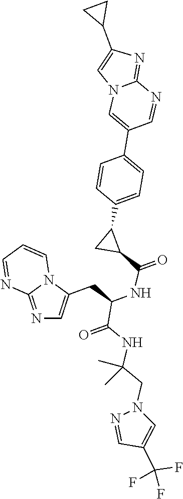 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nc(C4CC4)cn3c2)cc1] | 697 (M + H) |
| 77 | (1S,2S)-2-(4-(2- (Cyclobutylmethyl)-2H- pyrazolo[3,4-b]pyridin-5- yl)phenyl)-N-((R)-3- (imidazo[1,2-a]pyrimidin-3-yl)- 1-((2-methyl-1-(4- (trifluoromethyl)-1H-pyrazol-1- yl)propan-2-yl)amino)-1- oxopropan-2-yl)cyclopropane-1- carboxamide | 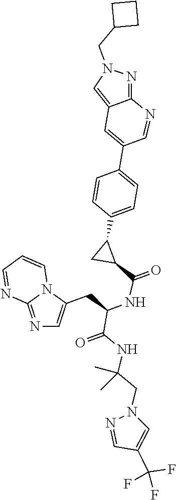 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nn(CC4CCC4)cc3c2)cc1] | 725 (M + H) |
| 78 | (1S,2S)-2-(4-(2- Cyclobutylimidazo[1,2- a]pyrimidin-6-yl)phenyl)-N-((R)- 3-(imidazo[1,2-a]pyrimidin-3- yl)-1-((2-methyl-1-(4- (trifluoromethyl)-1H-pyrazol-1- yl)propan-2-yl)amino)-1- oxopropan-2-yl)cyclopropane-1- carboxamide | 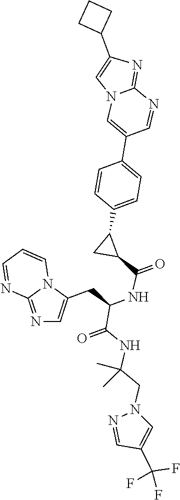 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nc(C4CCC4)cn3c2)cc1] | 711 (M + H) |
| 79 | (1S,2S)-2-(4-(2- (Difluoromethyl)imidazo[1,2- a]pyrimidin-6-yl)phenyl)-N-((R)- 3-(imidazo[1,2-a]pyrimidin-3- yl)-1-((2-methyl-1-(4- (trifluoromethyl)-1H-pyrazol-1- yl)propan-2-yl)amino)-1- oxopropan-2-yl)cyclopropane-1- carboxamide | 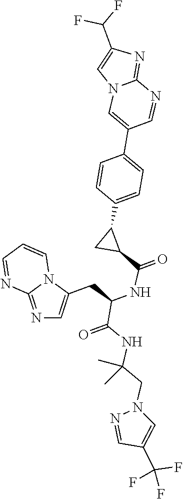 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nc(C(F)F)cn3c2)cc1] | 707 (M + H) |
| 80 | (1S,2S)-2-(4-(2-(1,1- Difluoroethyl)imidazo[1,2- a]pyrimidin-6-yl)phenyl)-N-((R)- 3-(imidazo[1,2-a]pyrimidin-3- yl)-1-((2-methyl-1-(4- (trifluoromethyl)-1H-pyrazol-1- yl)propan-2-yl)amino)-1- oxopropan-2-yl)cyclopropane-1- carboxamide | 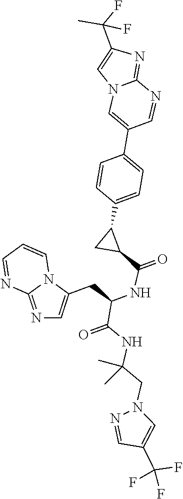 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nc(C(C)(F)F)cn3c2)cc1] | 721 (M + H) |
| 81 | (1S,2S)-N-((R)-3-(Imidazo[1,2- a]pyrimidin-3-yl)-1-((2-methyl- 1-(4-(trifluoromethyl)-1H- pyrazol-1-yl)propan-2-yl)amino)- 1-oxopropan-2-yl)-2-(4-(2-(1- methylcyclopropyl)imidazo[1,2- a]pyrimidin-6- yl)phenyl)cyclopropane-1- carboxamide | 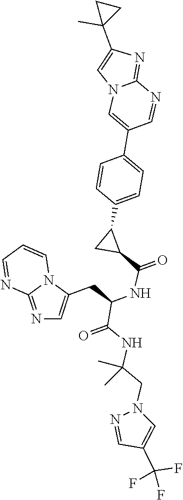 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nc(C4(C)CC4)cn3c2)cc1] | 711 (M + H) |
| 82 | (1S,2S)-2-(4-(2- Ethylimidazo[1,2-a]pyrimidin-6- yl)phenyl)-N-((R)-3- (imidazo[1,2-a]pyrimidin-3-yl)- 1-((2-methyl-1-(4- (trifluoromethyl)-1H-pyrazol-1- yl)propan-2-yl)amino)-1- oxopropan-2-yl)cyclopropane-1- carboxamide | 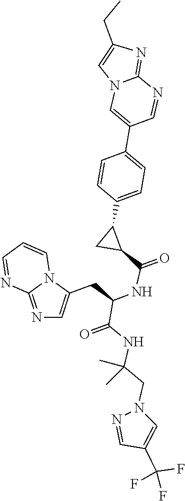 [SMILES: CCc1cn2cc(-c3ccc([C@H]4C[C@@H]4C(=O)N[C@H](Cc4cnc5ncccn45)C(=O)NC(C)(C)Cn4cc(C(F)(F)F)cn4)cc3)cnc2n1] | 685 (M + H) |
| 83 | (1S,2S)-N-((R)-3-(Imidazo[1,2- a]pyrimidin-3-yl)-1-((2-methyl- 1-(4-(trifluoromethyl)-1H- pyrazol-1-yl)propan-2-yl)amino)- 1-oxopropan-2-yl)-2-(4-(2- methylimidazo[1,2-a]pyrimidin- 6-yl)phenyl)cyclopropane-1- carboxamide | 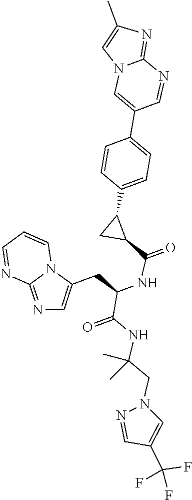 [SMILES: Cc1cn2cc(-c3ccc([C@H]4C[C@@H]4C(=O)N[C@H](Cc4cnc5ncccn45)C(=O)NC(C)(C)Cn4cc(C(F)(F)F)cn4)cc3)cnc2n1] | 671 (M + H) |
| 84 | (1S,2S)-2-(4-(2-Cyclobutyl-2H- pyrazolo[3,4-b]pyridin-5- yl)phenyl)-N-((R)-3- (imidazo[1,2-a]pyrimidin-3-yl)- 1-((2-methyl-1-(4- (trifluoromethyl)-1H-pyrazol-1- yl)propan-2-yl)amino)-1- oxopropan-2-yl)cyclopropane-1- carboxamide | 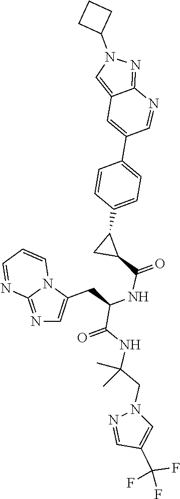 [SMILES: CC(C)(Cn1cc(C(F)(F)F)cn1)NC(=O)[C@@H](Cc1cnc2ncccn12)NC(=O)[C@H]1C[C@@H]1c1ccc(-c2cnc3nn(C4CCC4)cc3c2)cc1] | 711 (M + H) |
| 85 | (1S,2S)-N-((R)-1-((1-(5- Fluoropyridin-2-yl)-2- methylpropan-2-yl)amino)-3- (imidazo[1,2-a]pyrimidin-3-yl)- 1-oxopropan-2-yl)-2-(4-(2- (trifluoromethyl)imidazo[1,2- a]pyrimidin-6- yl)phenyl)cyclopropane-1- carboxamide | 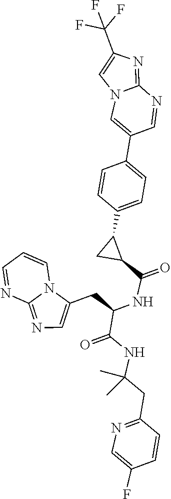 [SMILES: CC(C)(Cc1ccc(F)cn1)NC(=O)[C@@H](Cc1cnc2ncccn12)NC(=O)[C@H]1C[C@@H]1c1ccc(-c2cnc3nc(C(F)(F)F)cn3c2)cc1] | 686 (M + H) |
| 86 | (1S,2S)-N-((R)-1-((1-(4- Fuorophenyl)-2-methylpropan-2- yl)amino)-3-(imidazo[1,2- a]pyrimidin-3-yl)-1-oxopropan- 2-yl)-2-(4-(2- (trifluoromethyl)imidazo[1,2- a]pyrimidin-6- yl)phenyl)cyclopropane-1- carboxamide | 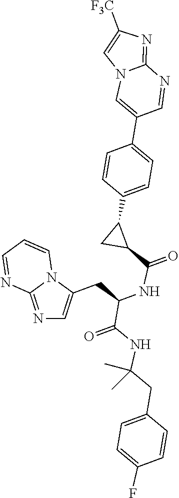 [SMILES: CC(C)(Cc1ccc(F)cc1)NC(=O)[C@@H](Cc1cnc2ncccn12)NC(=O)[C@H]1C[C@@H]1c1ccc(-c2cnc3nc(C(F)(F)F)cn3c2)cc1] | 685 (M + H) |
| 87 | (1S,2S)-N-((R)-1-((1-(4- Fluorophenyl)-2-methylpropan-2- yl)amino)-3-(imidazo[1,2- a]pyrimidin-3-yl)-1-oxopropan- 2-yl)-2-(4-(imidazo[1,2- a]pyrimidin-6- yl)phenyl)cyclopropane-1- carboxamide | 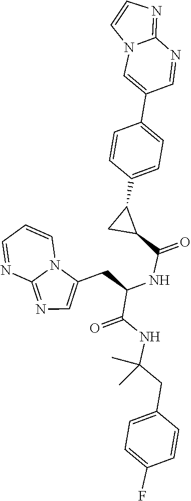 [SMILES: CC(C)(Cc1ccc(F)cc1)NC(=O)[C@@H](Cc1cnc2ncccn12)NC(=O)[C@H]1C[C@@H]1c1ccc(-c2cnc3nccn3c2)cc1] | 617 (M + H) |
| 88 | (1S,2S)-2-(4-(2- (Difluoromethyl)-2H- pyrazolo[3,4-b]pyridin-5- yl)phenyl)-N-methyl-N-((R)-1- ((2-methyl-1-(5-(1-methyl-6-oxo- 1,6-dihydropyridin-3-yl)-3- (trifluoromethyl)-1H-pyrazol-1- yl)propan-2-yl)amino)-3-(1- methyl-1H-pyrazol-4-yl)-1- oxopropan-2-yl)cyclopropane-1- carboxamide | 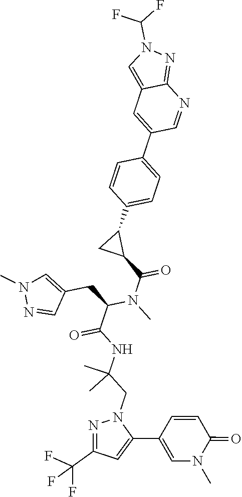 [SMILES: CN(C(=O)[C@H]1C[C@@H]1c1ccc(-c2cnc3nn(C(F)F)cc3c2)cc1)[C@H](Cc1cnn(C)c1)C(=O)NC(C)(C)Cn1nc(C(F)(F)F)cc1-c1ccc(=O)n(C)c1] | 791 (M + H) |
| 89 | (1S,2S)-N-Methyl-N-((R)-1-((2- methyl-1-(5-(1-methyl-6-oxo- 1,6-dihydropyridin-3-yl)-3- (trifluoromethyl)-1H-pyrazol-1- yl)propan-2-yl)amino)-3-(1- methyl-1H-pyrazol-4-yl)-1- oxopropan-2-yl)-2-(4-(2- (trifluoromethyl)-2H- pyrazolo[3,4-b]pyridin-5- yl)phenyl)cyclopropane-1- carboxamide | 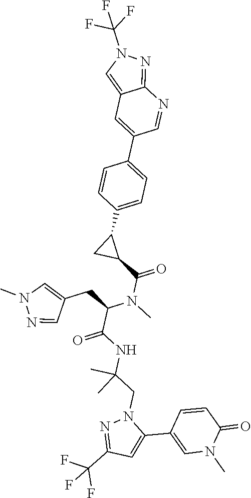 [SMILES: CN(C(=O)[C@H]1C[C@@H]1c1ccc(-c2cnc3nn(C(F)(F)F)cc3c2)cc1)[C@H](Cc1cnn(C)c1)C(=O)NC(C)(C)Cn1nc(C(F)(F)F)cc1-c1ccc(=O)n(C)c1] | 809 (M + H) |
| 90 | (1S,2S)-2-(4-(2- (Methoxymethyl)imidazo[1,2- a]pyrimidin-6-yl)phenyl)-N- methyl-N-((R)-1-((2-methyl-1-(5- (1-methyl-6-oxo-1,6- dihydropyridin-3-yl)-3- (trifluoromethyl)-1H-pyrazol-1- yl)propan-2-yl)amino)-3-(1- methyl-1H-pyrazol-4-yl)-1- oxopropan-2-yl)cyclopropane-1- carboxamide | 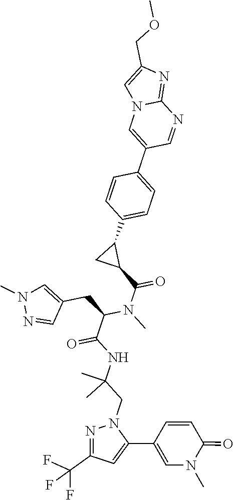 [SMILES: COCc1cn2cc(-c3ccc([C@H]4C[C@@H]4C(=O)N(C)[C@H](Cc4cnn(C)c4)C(=O)NC(C)(C)Cn4nc(C(F)(F)F)cc4-c4ccc(=O)n(C)c4)cc3)cnc2n1] | 785 (M + H) |
| 91 | (1S,2S)-N-Methyl-N-((R)-1-((2- methyl-1-(5-(1-methyl-6-oxo- 1,6-dihydropyridin-3-yl)-3- (trifluoromethyl)-1H-pyrazol-1- yl)propan-2-yl)amino)-3-(1- methyl-1H-pyrazol-4-yl)-1- oxopropan-2-yl)-2-(4-(2- methylimidazo[1,2-a]pyrimidin- 6-yl)phenyl)cyclopropane-1- carboxamide | 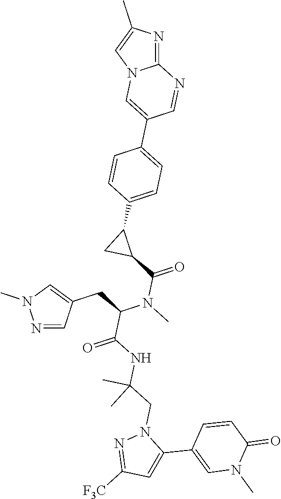 [SMILES: Cc1cn2cc(-c3ccc([C@H]4C[C@@H]4C(=O)N(C)[C@H](Cc4cnn(C)c4)C(=O)NC(C)(C)Cn4nc(C(F)(F)F)cc4-c4ccc(=O)n(C)c4)cc3)cnc2n1] | 755 (M + H) |

### Example 92

### (1S,2S)-N—((R)-1-((1-(5-(2H-1,2,3-Triazol-4-yl)-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

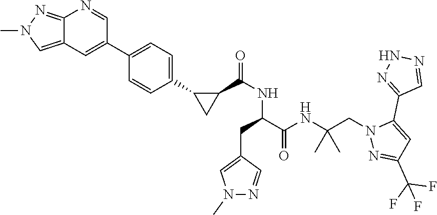 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cn[nH]n2)cn1]

A solution of (1S,2S)-N—((R)-1-((1-(5-(2-(methoxymethyl)-2H-1,2,3-triazol-4-yl)-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide (67.3 mg, 90.4 μmol) in TFA (904 μL) was stirred at RT for 22 h. The reaction mixture was concentrated under a stream of N₂, and the residue was purified by reversed phase prep-HPLC (XSelect® CSH C18, 30×150 mm, 5 μm, 40 mL/min) eluting with 5-95% ACN in water (with 0.1% FA) over 20 min to obtain the title compound (12.3 mg, 19%) as an off-white powder. ES-MS m/z 701.4 (M+H).

### Example 93

### (1S,2S)-N—((R)-1-((1-(5-(2H-1,2,3-Triazol-4-yl)-3-(trifluoromethyl)-1H-pyrazol-1-yl)-2-methylpropan-2-yl)amino)-3-(1-methyl-1H-pyrazol-4-yl)-1-oxopropan-2-yl)-2-(2-fluoro-4-(2-methyl-2H-pyrazolo[3,4-b]pyridin-5-yl)phenyl)cyclopropane-1-carboxamide

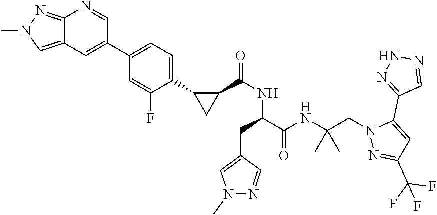 [SMILES: Cn1cc(C[C@@H](NC(=O)[C@H]2C[C@@H]2c2ccc(-c3cnc4nn(C)cc4c3)cc2F)C(=O)NC(C)(C)Cn2nc(C(F)(F)F)cc2-c2cn[nH]n2)cn1]

A solution of (1S,2S)-2-(2-fluoro-4-(2-methyl-2H-pyrazolo[3,4-b]pyridin-5-yl)phenyl)-N-((2R)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-3-(1-methyl-1H-pyrazol-4-yl)-1-oxopropan-2-yl)cyclopropane-1-carboxamide (170 mg, 212 μmol) in DCM (2 mL) was treated with 4M HCl in 1,4-dioxane (635 μL, 2.54 mmol) dropwise. The resulting mixture was stirred at RT for 2 h, then concentrated under reduced pressure. The residue was purified by reversed phase prep-HPLC (Kinetex® EVO C18, 30×250 mm, 5 μm, 60 mL/min) using a gradient of 25-45% ACN in 10 mM aq. NH₄HCO₃, pH=10 (with 5% MeOH) over 10 min to obtain the title compound (152 mg, 54%) as a white powder. ES-MS m/z 719.6 (M+H).

### Example 94

### (1S,2S)-N—((R)-1-((1-(5-(2H-1,2,3-Triazol-4-yl)-3-(trifluoromethyl)-1H-pyrazol-1-yl)-2-methylpropan-2-yl)amino)-3-(1-methyl-1H-pyrazol-4-yl)-1-oxopropan-2-yl)-2-(4-(7-methyl-1,8-naphthyridin-3-yl)phenyl)cyclopropane-1-carboxamide

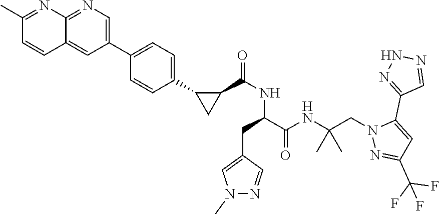 [SMILES: Cc1ccc2cc(-c3ccc([C@H]4C[C@@H]4C(=O)N[C@H](Cc4cnn(C)c4)C(=O)NC(C)(C)Cn4nc(C(F)(F)F)cc4-c4cn[nH]n4)cc3)cnc2n1]

The title compound was prepared as described in Example 83 using (1S,2S)-2-(4-(7-Methyl-1,8-naphthyridin-3-yl)phenyl)-N-((2R)-1-((2-methyl-1-(5-(2-(tetrahydro-2H-pyran-2-yl)-2H-1,2,3-triazol-4-yl)-3-(trifluoromethyl)-1H-pyrazol-1-yl)propan-2-yl)amino)-3-(1-methyl-1H-pyrazol-4-yl)-1-oxopropan-2-yl)cyclopropane-1-carboxamide with purification by reversed phase prep-HPLC (Kinetex® EVO C18, 30×100 mm, 5 μm, 45° C., 85 mL/min) using a gradient of 23-58% ACN in water (with 0.1% FA) over 6 min. ES-MS m/z 712.6 (M+H).

### Example 95

### (R)—N-(1-(5-(2H-1,2,3-Triazol-4-yl)-3-(trifluoromethyl)-1H-pyrazol-1-yl)-2-methylpropan-2-yl)-3-(1-methyl-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide

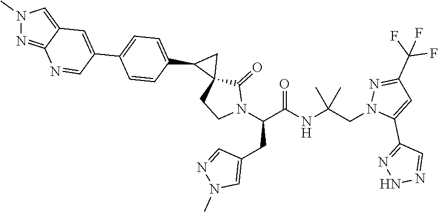 [SMILES: Cn1cc(C[C@H](C(=O)NC(C)(C)Cn2nc(C(F)(F)F)cc2-c2cn[nH]n2)N2CC[C@@]3(C[C@@H]3c3ccc(-c4cnc5nn(C)cc5c4)cc3)C2=O)cn1]

A mixture of (R)—N-(2-methyl-1-(5-(2-(tetrahydro-2H-pyran-2-yl)-2H-1,2,3-triazol-4-yl)-3-(trifluoromethyl)-1H-pyrazol-1-yl)propan-2-yl)-3-(1-methyl-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide (340 mg, 419 μmol) and TFA (1.62 mL, 21.0 mmol) was stirred at RT for 2.5 h. The reaction mixture was concentrated under reduced pressure, diluted with DCM (2 mL) and re-concentrated. The residue was purified by reversed phase prep-HPLC (Kinetex® EVO C18, 30× 100 mm, 5 μm, 85 mL/min, 3 injections) using a gradient of 14-48% ACN in 10 mM aq. NH₄HCO₃, pH=10 (with 5% MeOH) over 6 min to obtain the title compound (158 mg, 52%) as a white solid. ES-MS m/z 727.6 (M+H).

### Example 96

### (1S,2S)-N—((R)-1-((2-methyl-1-(5-(4,5,6,7-tetrahydropyrazolo[1,5-a]pyrazin-3-yl)-3-(trifluoromethyl)-1H-pyrazol-1-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide

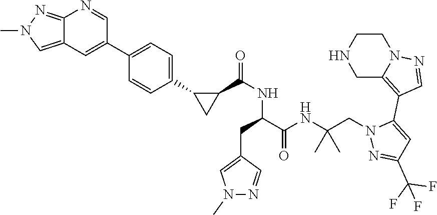 [SMILES: Cn1cc(C[C@@H](NC(=O)[C@H]2C[C@@H]2c2ccc(-c3cnc4nn(C)cc4c3)cc2)C(=O)NC(C)(C)Cn2nc(C(F)(F)F)cc2-c2cnn3c2CNCC3)cn1]

A solution of tert-butyl 3-(1-(2-methyl-2-((R)-3-(1-methyl-1H-pyrazol-4-yl)-2-((1S,2S)-2-(4-(2-methyl-2H-pyrazolo[3,4-b]pyridin-5-yl)phenyl)cyclopropane-1-carboxamido)propanamido) propyl)-3-(trifluoromethyl)-1H-pyrazol-5-yl)-6,7-dihydropyrazolo[1,5-a]pyrazine-5 (4H)-carboxylate (80.0 mg, 93.6 μmol) in EtOAc (624 μL) was treated with 4M HCl in 1,4-dioxane (281 μL, 1.12 mmol). After stirring at RT for 4 days, the reaction mixture was concentrated under reduced pressure, and the residue was purified by reversed phase prep-HPLC (XSelect® CSH C18, 30×150 mm, 5 μm, 40 mL/min) eluting with 5-95% ACN in water (with 0.1% FA) over 20 min to obtain the title compound (22.5 mg, 32%) as an off-white powder. ES-MS m/z 755.4 (M+H).

### Example 97

### (1S,2S)-N—((R)-1-((1-(4-Fluorophenyl)-2-methylpropan-2-yl)amino)-3-(1-methyl-1H-pyrazol-4-yl)-1-oxopropan-2-yl)-2-(4-(3-(4-fluorophenyl) ureido)phenyl)cyclopropane-1-carboxamide

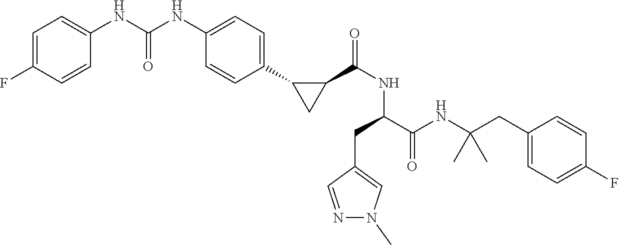 [SMILES: Cn1cc(C[C@@H](NC(=O)[C@H]2C[C@@H]2c2ccc(NC(=O)Nc3ccc(F)cc3)cc2)C(=O)NC(C)(C)Cc2ccc(F)cc2)cn1]

A mixture of (1S,2S)-2-(4-aminophenyl)-N—((R)-1-((1-(4-fluorophenyl)-2-methylpropan-2-yl)amino)-3-(1-methyl-1H-pyrazol-4-yl)-1-oxopropan-2-yl)cyclopropane-1-carboxamide hydrochloride (100 mg, 146 μmol, 75% purity) in DMF (3 mL) was treated with TEA (61.0 μL, 438 μmol) and 4-fluorophenyl isocyanate (30.0 mg, 219 μmol). The resulting mixture was stirred at 25° C. for 2 h then diluted with water (10 mL) and extracted with EtOAc (2× 10 mL). The organic layers were combined, washed with sat. aq. NaCl (10 mL), dried over Na₂SO₄, filtered, and concentrated under reduced pressure. The residue was purified by reversed phase prep-HPLC (Xtimate® C18, 40×150 mm, 10 μm) using a gradient of 28-68% ACN in water (with 0.1% FA) over 25 min to obtain the title compound (39 mg, 44%) as a white solid. ES-MS (m z) 615.1 (M+H).

### Example 98

### (R)—N-(2-Methyl-1-(5-(1-methyl-6-oxo-1,6-dihydropyridin-3-yl)-3-(trifluoromethyl)-1H-pyrazol-1-yl)propan-2-yl)-3-(1-methyl-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide

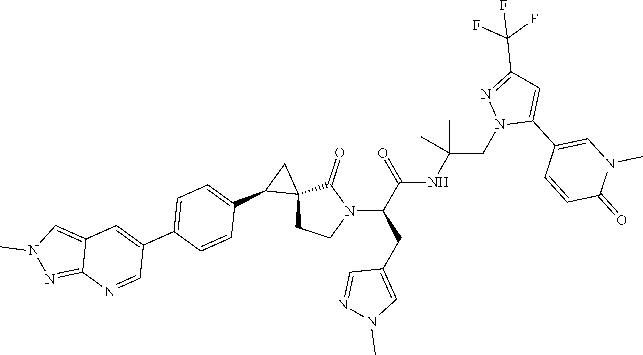

A mixture of (1R,2R)-1-(2-chloroethyl)-N—((R)-1-((2-methyl-1-(5-(1-methyl-6-oxo-1,6-dihydropyridin-3-yl)-3-(trifluoromethyl)-1H-pyrazol-1-yl)propan-2-yl)amino)-3-(1-methyl-1H-pyrazol-4-yl)-1-oxopropan-2-yl)-2-(4-(2-methyl-2H-pyrazolo[3,4-b]pyridin-5-yl)phenyl)cyclopropane-1-carboxamide (849 mg, 1.06 mmol), TBAI (39 mg, 106 μmol) and Cs₂CO₃ (1.38 g, 4.23 mmol) in NMP (11 mL) was stirred overnight at RT. The reaction mixture was added dropwise to rapidly stirring water (150 mL). The precipitate was collected by suction filtration and purified by reversed phase prep-HPLC (XSelect® CSH C18, 50×250 mm, 5 μm, 100 mL/min) eluting with 5-95% ACN in water (with 0.1% FA) over 32 min to obtain the title compound (282 mg, 35%). ES-MS m/z 767.6 (M+H).

### Example 99

### (R)-3-(1-(Difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-((1S,3R)-1-(2-fluoro-4-(2-methyl-2H-pyrazolo[3,4-b]pyridin-5-yl)phenyl)-4-oxo-5-azaspiro[2.4]heptan-5-yl)propanamide, Isomer 1

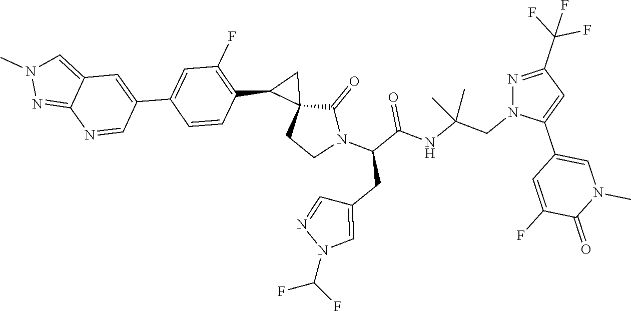

A mixture of (1R,2S)-1-(2-chloroethyl)-N—((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)-2-(2-fluoro-4-(2-methyl-2H-pyrazolo[3,4-b]pyridin-5-yl)phenyl)cyclopropane-1-carboxamide (519 mg, 593 μmol) in NMP (6 mL) was treated with Cs₂CO₃ (386 mg, 1.18 mmol). After stirring overnight at RT, the reaction mixture was diluted with water and extracted with EtOAc (3×). The organic layers were combined, washed with water (3×), dried over Na₂SO₄, filtered, and concentrated under reduced pressure. The residue was purified by reversed phase prep-HPLC (Luna® C18, 50×250 mm, 10 μm) using a gradient of 35-65% ACN in 10 mM aq. NH₄HCO₃, pH=10 (with 5% MeOH) over 22.5 min to obtain the title compound (253 mg, 51%) as a white solid. ES-MS m/z 839 (M+H).

### Example 100

### (2R)-3-(1-(Difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-((1S,3R)-1-(2-fluoro-4-(2-methylimidazo[1,2-a]pyrimidin-6-yl)phenyl)-4-oxo-5-azaspiro[2.4]heptan-5-yl)propanamide

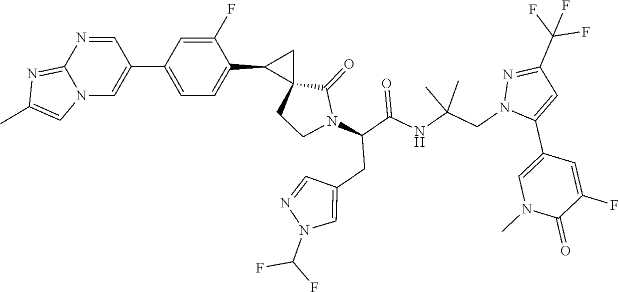 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4cc(F)c(=O)n(C)c4)C5=O)c(F)c3)cnc2n1]

A stirring mixture of 6-bromo-2-methylimidazo[1,2-a]pyrimidine (979 mg, 4.62 mmol), Cs₂CO₃ (4.01 g, 12.3 mmol) in 1,4-dioxane (30 mL) and water (5 mL) was sparged with N₂ for 5 min. Then Pd(dppf)Cl₂ (377 mg, 462 μmol) was added and sparged with N₂ for 5 min. Then a crude mixture of (2R)-3-(1-(difluoromethyl)-1H-pyrazol-4-yl)-2-(1S,3R)-(1-(4-(5,5-dimethyl-1,3,2-dioxaborinan-2-yl)-2-fluorophenyl)-4-oxo-5-azaspiro[2.4]heptan-5-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide from Preparation 328 was added and heated to 90° C. for 1 h. The reaction mixture was cooled to RT, diluted with EtOAc (200 mL), dried over MgSO₄, filtered through diatomaceous earth pad, and washed with EtOAc (200 mL). The filtrate was concentrated under reduced pressure. The residue was purified by silica gel chromatography using a gradient of 0-10% 7 M ammonia in MeOH in EtOAc to obtain 2.5 g red foam. The red foam was dissolved in EtOAc, treated with 1.5 g SiliaMetS® metal scavenger resin, stirred for 3 h, filtered and concentrated to give 2.4 g light-yellow foam. The light-yellow foam was purified by reversed phase flash chromatography using a gradient of 35-65% ACN in 10 mM aq. NH₄HCO₃, pH=10 (with 5% MeOH) over 16 min to obtain the title compound (1.67 g, 64%) as off-white powder. ES-MS m/z 839.6 (M+H).

The compounds in the following table were prepared as described in Example 100 using the fresh-made boron ester crude mixture and the appropriate aryl bromide coupling partners. Reactants can be added in different orders or in differing equivalency. Differing methods can be used to work up or purify the compounds (normal phase, high or low pH prep-HPLC). Such variances would be apparent to one skilled in the art.

TABLE 35

| Ex- | | | ES-MS |
|---|---|---|---|
| ample | Chemical Name | Structure | m/z |
| 101^{a,b} | (R)-N-(1-(5-(2- Cyclopropylpyrimidin-5-yl)- 3-(trifluoromethyl)-1H- pyrazol-1-yl)-2-methylpropan- 2-yl)-3-(1-(difluoromethyl)- 1H-pyrazol-4-yl)-2-((1R,3R)- 1-(4-(2-methylimidazo[1,2- a]pyrimidin-6-yl)phenyl)-4- oxo-5-azaspiro[2.4]heptan-5- yl)propanamide, Isomer 1 | 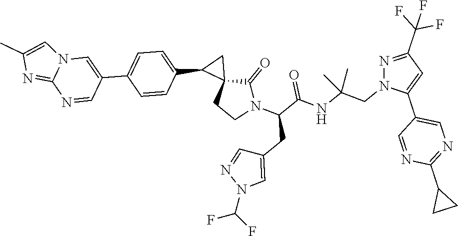 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4cnc(C6CC6)nc4)C5=O)cc3)cnc2n1] | 814.4 (M + H) |
| 102^{a,c} | (R)-3-(1-(Difluoromethyl)- 1H-pyrazol-4-yl)-N-(2- methyl-1-(3-(trifluoromethyl)- 5-(2- (trifluoromethyl)pyrimidin-5- yl)-1H-pyrazol-1-yl)propan-2- yl)-2-((1R,3R)-1-(4-(2- methylimidazo[1,2- a]pyrimidin-6-yl)phenyl)-4- oxo-5-azaspiro[2.4]heptan-5- yl)propanamide, Isomer 1 | 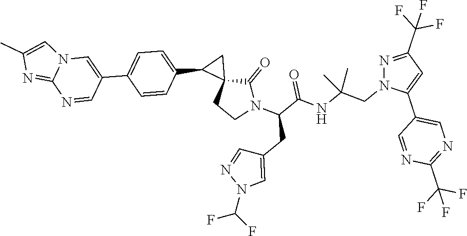 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4cnc(C(F)(F)F)nc4)C5=O)cc3)cnc2n1] | 842.4 (M + H) |
| 103^{a,c} | (2R)-3-(1-(Difluoromethyl)- 1H-pyrazol-4-yl)-N-(2- methyl-1-(3-(trifluoromethyl)- 5-(2- (trifluoromethyl)pyrimidin-5- yl)-1H-pyrazol-1-yl)propan-2- yl)-2-((1R,3R)-1-(4-(6- methyl-7-oxo-4a,6,7,7a- tetrahydro-5H-pyrrolo[3,4- b]pyridin-3-yl)phenyl)-4-oxo- 5-azaspiro[2.4]heptan-5- yl)propanamide, Isomer 1 | 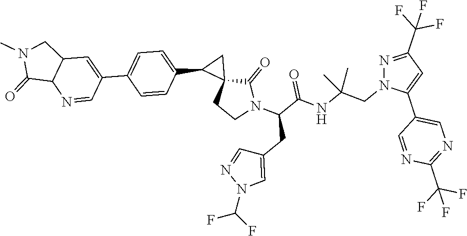 [SMILES: CN1CC2C=C(c3ccc([C@H]4C[C@@]45CCN([C@H](Cc4cnn(C(F)F)c4)C(=O)NC(C)(C)Cn4nc(C(F)(F)F)cc4-c4cnc(C(F)(F)F)nc4)C5=O)cc3)C=NC2C1=O] | 857.4 (M + H) |
| 104 | (R)-3-(1-(Difluoromethyl)- 1H-pyrazol-4-yl)-2-((1R,3R)- 1-(4-(7-(2-hydroxypropan-2- yl)-1,8-naphthyridin-3- yl)phenyl)-4-oxo-5- azaspiro[2.4]heptan-5-yl)-N- (2-methyl-1-(5-(1-methyl-6- oxo-1,6-dihydropyridin-3-yl)- 3-(trifluoromethyl)-1H- pyrazol-1-yl)propan-2- yl)propanamide, Isomer 1 | 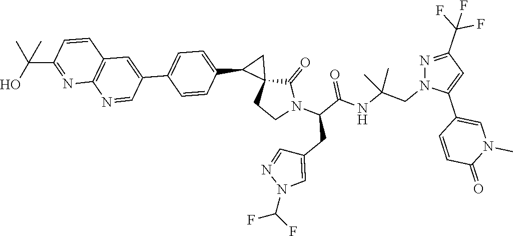 [SMILES: Cn1cc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nc(C(C)(C)O)ccc5c4)cc3)C2=O)ccc1=O] | 858.3 (M + H) |
| 105 | (R)-3-(1-(Difluoromethyl)- 1H-pyrazol-4-yl)-2-((1R,3R)- 1-(4-(6-methoxy-7-methyl- 1,8-naphthyridin-3- yl)phenyl)-4-oxo-5- azaspiro[2.4]heptan-5-yl)-N- (2-methyl-1-(5-(1-methyl-6- oxo-1,6-dihydropyridin-3-yl)- 3-(trifluoromethyl)-1H- pyrazol-1-yl)propan-2- yl)propanamide, Isomer 1 | 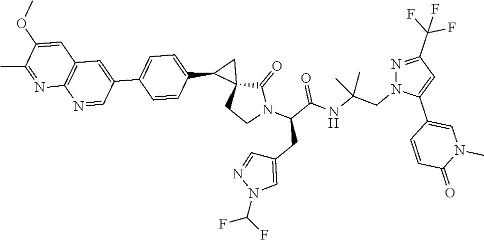 | 844.3 (M + H) |
| 106 | (2R)-3-(1-(Difluoromethyl)- 1H-pyrazol-4-yl)-N-(1-(5-(5- fluoro-1-methyl-6-oxo-1,6- dihydropyridin-3-yl)-3- (trifluoromethyl)-1H-pyrazol- 1-yl)-2-methylpropan-2-yl)-2- ((1S,3R)-1-(2-fluoro-4-(2-(1- methylazetidin-3-yl)-2H- pyrazolo[3,4-b]pyridin-5- yl)phenyl)-4-oxo-5- azaspiro[2.4]heptan-5- yl)propanamide, Isomer 1 | 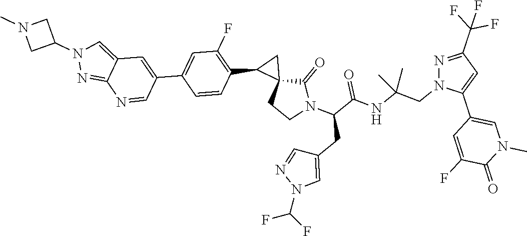 | 894.4 (M + H) |
| 107^{d} | (2R)-3-(1-(Difluoromethyl)- 1H-pyrazol-4-yl)-N-(1-(5-(5- fluoro-1-methyl-6-oxo-1,6- dihydropyridin-3-yl)-3- (trifluoromethyl)-1H-pyrazol- 1-yl)-2-methylpropan-2-yl)-2- ((1S,3R)-1-(2-fluoro-4-(2-(2- hydroxyethyl)-2H- pyrazolo[3,4-b]pyridin-5- yl)phenyl)-4-oxo-5- azaspiro[2.4]heptan-5- yl)propanamide, Isomer 1 | 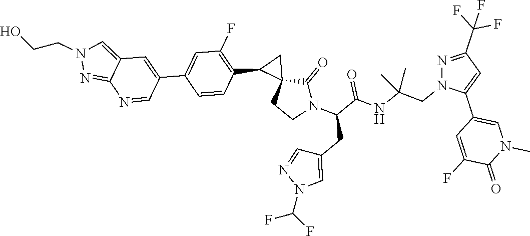 [SMILES: Cn1cc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nn(CCO)cc5c4)cc3F)C2=O)cc(F)c1=O] | 869.6 (M + H) |

^{a}The reaction mixture was concentrated under steam of nitrogen. The residue was dissolved in 5 mL EtOAc and treated with 2 g Silicycle SiliMetS ® Thiol metal scavenger, stirred for 15 mins and filtered. The residue was washed with 30 mL EtOAc and concentrated under reduced pressure. The residue was dissolved in DMF and purified on reversed phase;
^{b}purified by XSelect CSH (C18, 30 mm × 150 mm, 5 um, 40 mL/min) using a gradient of 5-95% ACN in water with 0.1% FA;
^{c}purified by XSelect CSH (C18, 30 mm × 250 mm, 5 um, 40 mL/min) using a gradient of 5-95% ACN in water with 10 mM NH₄OAc;
^{d}The reaction mixture was diluted with EtOAc, dried over anhydrous sodium sulfate, filtered through diatomaceous earth, washed with 5% MeOH in EtOAc. The filtrate was concentrated under reduced pressure. The residue was purified by similar method.

### Example 108

### (2R)-3-(1-(Difluoromethyl)-1H-pyrazol-4-yl)-2-((trans)-1-(2-(difluoromethyl)-4-(2-methylimidazo[1,2-a]pyrimidin-6-yl)phenyl)-4-oxo-5-azaspiro[2.4]heptan-5-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide, isomer 1

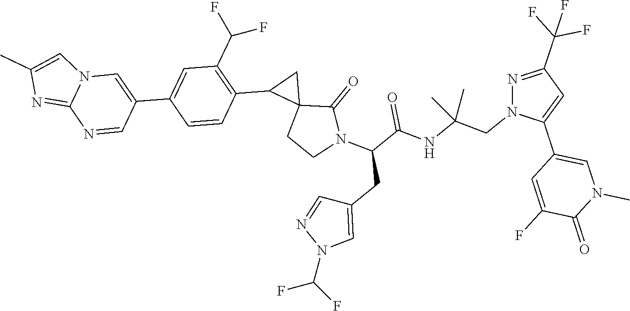

(R)-3-(1-(Difluoromethyl)-1H-pyrazol-4-yl)-2-((trans)-1-(2-(difluoromethyl)-4-(4,4,5,5-tetramethyl-1,3,2-dioxaborolan-2-yl)phenyl)-4-oxo-5-azaspiro[2.4]heptan-5-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (120 mg, 139 μmol) in 1,4-dioxane (5 mL), 6-bromo-2-methylimidazo[1,2-a]pyrimidine (100 mg, 472 μmol), PdCl₂(dppf)-CH₂Cl₂ (40 mg, 49 μmol), Cesium Carbonate (200 mg, 614 μmol) and water (2.0 mL). The mixture was purged under N₂ for 3 mins, then heated the reaction mixture to 90° C. for 1 h. The reaction mixture was concentrated under a stream of N₂ overnight. The residue was dissolved in DMF (3 mL), filtered by syringe filter, and purified by UPLC CSH (XSelect, C18, 50 mm×250 mm×5 μm, 100 mL/min) using a gradient of 5-95% ACN/water (w/0.1% FA), followed by chiral SFC separation (Chiralpak IC 30×250 mm×5 μM, 34 mL/min), eluting with 10 mM NH₄OAc in MeOH to afford the title compound (8 mg, 7%) as the first eluting diastereomer. ES-MS m/z 871.4 (M+H).

### Example 109

### (2R)-3-(1-(Difluoromethyl)-1H-pyrazol-4-yl)-2-((trans)-1-(2-(difluoromethyl)-4-(2-methyl-2H-pyrazolo[3,4-b]pyridin-5-yl)phenyl)-4-oxo-5-azaspiro[2.4]heptan-5-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide, Isomer 2

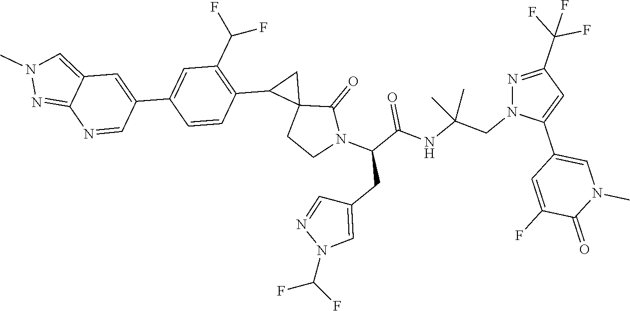

2-Methyl-5-(4,4,5,5-tetramethyl-1,3,2-dioxaborolan-2-yl)-2H-pyrazolo[3,4-b]pyridine (57.0 mg, 220 μmol), (R)-2-((trans)-1-(4-bromo-2-(difluoromethyl)phenyl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (120 mg, 147 μmol), Pd(dppf)-dichloride adduct (32.2 mg, 44.0 μmol), and cesium carbonate (115 mg, 353 μmol) were dissolved in 1,4-Dioxane (4 mL) and water (0.4 mL). The mixture was then stirred at RT for roughly 5 min while purging with N₂, then the reaction mixture was heated to 90° C. and allowed to stir for 2 h. The reaction mixture was cooled, Silicycle SiliMetS® Thiol metal scavenger was added, the resulting mixture was stirred for 5 min and then filtered. The residue was dissolved in DMF (3 mL), filtered by syringe filter, and purified by UPLC CSH (XSelect, C18, 50 mm×250 mm×5 μm, 100 mL/min) using a gradient of 5-95% ACN/water (w/0.1% FA), followed by chiral SFC separation (Chiralpak IC 30×250 mm×5 μm, 42 mL/min), eluting with ACN to afford the title compound (26.4 mg, 19%) as the second eluting diastereomer. ES-MS m/z 871.4 (M+H).

The compounds in the following table were prepared as described in Example 109 using the appropriate boron ester or boronic acid and the appropriate aryl bromide coupling partners. Reactants can be added in different orders or in differing equivalency. Differing methods can be used to work up or purify the compounds (normal phase, high or low pH prep-HPLC). Such variances would be apparent to one skilled in the art.

TABLE 36

| Ex- | | | ES-MS |
|---|---|---|---|
| ample | Chemical Name | Structure | m/z |
| 110^{a,b} | (2R)-3-(1-(Difluoromethyl)- 1H-pyrazol-4-yl)-N-(2- methyl-1-(5-(2- methylpyrimidin-5-yl)-3- (trifluoromethyl)-1H-pyrazol- 1-yl)propan-2-yl)-2-((trans)- 1-(2-methyl-6-(2- methylimidazo[1,2- a]pyrimidin-6-yl)pyridin-3- yl)-4-oxo-5- azaspiro[2.4]heptan-5- yl)propanamide, isomer 1 | 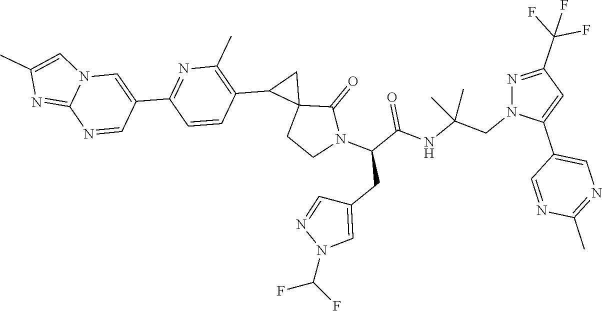 [SMILES: Cc1cn2cc(-c3ccc(C4CC45CCN([C@H](Cc4cnn(C(F)F)c4)C(=O)NC(C)(C)Cn4nc(C(F)(F)F)cc4-c4cnc(C)nc4)C5=O)c(C)n3)cnc2n1] | 803.6 (M + H) |
| 111^{a,c} | (2R)-3-(1-(Difluoromethyl)- 1H-pyrazol-4-yl)-N-(2- methyl-1-(5-(2- methylpyrimidin-5-yl)-3- (trifluoromethyl)-1H-pyrazol- 1-yl)propan-2-yl)-2-((trans)- 1-(2-methyl-6-(2-methyl-2H- pyrazolo[3,4-b]pyridin-5- yl)pyridin-3-yl)-4-oxo-5- azaspiro[2.4]heptan-5- yl)propanamide, isomer 1 | 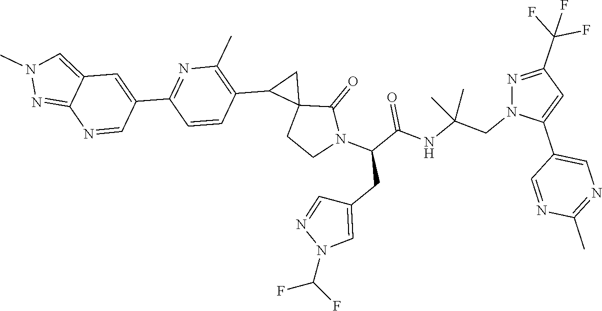 [SMILES: Cc1ncc(-c2cc(C(F)(F)F)nn2CC(C)(C)NC(=O)[C@@H](Cc2cnn(C(F)F)c2)N2CCC3(CC3c3ccc(-c4cnc5nn(C)cc5c4)nc3C)C2=O)cn1] | 803.6 (M + H) |

^{a}Purified on reversed phase XSelect CSH (C18, 50 mm × 250 mm, 5 um, 100 mL/min) using a gradient of 5-95% ACN in water with 10 mM NH₄OAc;
^{b}Purified by Chiralpak IK (50 mm × 250 mm, 5 um, 42 mL/min), eluting with 100% ACN, first eluting isomer;
^{c}Purified by Chiralpak IK (50 mm × 250 mm, 5 um, 52 mL/min), eluting with 100% ACN, second eluting isomer.

### Example 112

### (2R)-3-(1-(Difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)-2-((trans)-1-(6-(2-methyl-2H-pyrazolo[3,4-b]pyridin-5-yl)-2-(trifluoromethyl)pyridin-3-yl)-4-oxo-5-azaspiro[2.4]heptan-5-yl)propanamide, Isomer 1

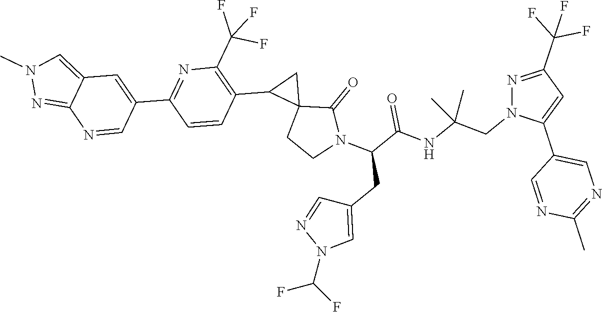 [SMILES: Cc1ncc(-c2cc(C(F)(F)F)nn2CC(C)(C)NC(=O)[C@@H](Cc2cnn(C(F)F)c2)N2CCC3(CC3c3ccc(-c4cnc5nn(C)cc5c4)nc3C(F)(F)F)C2=O)cn1]

A 2-dram vial was charged with a stirbar, (R)-2-((trans)-1-(6-chloro-2-(trifluoromethyl)pyridin-3-yl)-4-oxo-5-azaspiro[2.4]heptan-5-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide (296 mg, 92% Wt, 358 μmol), (2-Methyl-2H-pyrazolo[3,4-b]pyridin-5-yl) boronic acid (132 mg, 746 μmol), sodium carbonate (133 mg, 1.25 mmol), bis(di-tert-butyl (4-dimethylaminophenyl)phosphine)dichloropalladium (II) (49.6 mg, 70.0 μmol), and MeOH (5 mL). The resulting mixture was sparged with argon while stirring at RT for 5 min, then stirred at 65° C. for 22.5 h. The reaction mixture was cooled to RT and SiliaMetS Thiol resin (Silicycle, 1.46 mmol/g, 500 mg) was added. The reaction mixture was stirred at RT for 15 min, filtered, and concentrated. The residue was resuspended in ACN (5 mL) and re-filtered. The filtrate was concentrated and the residue was purified by reversed phase prep-HPLC (XSelect CSH C18, 50 mm×250 mm, 5 μm, 100 mL/min), eluting with a gradient of 5-95% ACN in 0.1% aq. FA over 32 min to provide the title compound (107 mg, 34%) as a fluffy white solid. ES-MS m/z 857.6 (M+H).

The compounds in the following table were prepared as described in Example 112 using the appropriate boron ester or boronic acid and the appropriate aryl bromide coupling partners. Differing methods can be used to work up or purify the compounds (normal phase, high or low pH prep-HPLC). Such variances would be apparent to one skilled in the art.

TABLE 37

| Ex- | | | ES-MS |
|---|---|---|---|
| ample | Chemical Name | Structure | m/z |
| 113 | (2R)-3-(1- (Difluoromethyl)- 1H-pyrazol-4-yl)-N- (2-methyl-1-(5- (1-methyl-6-oxo- 1,6-dihydropyridin- 3-yl)-3- (trifluoromethyl)- 1H-pyrazol- 1-yl)propan-2-yl)- 2-((trans)- 1-(6-(2-methyl-2H- pyrazolo[3,4-b] pyridin-5-yl)- 2-(trifluoromethyl) pyridin-3-yl)-4-oxo-5- azaspiro[2.4]heptan-5- yl)propanamide, isomer 1 | 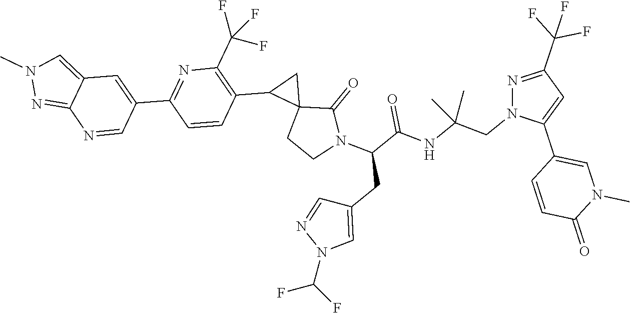 | 872.6 (M + H) |
| 114 | (2R)-2-((1S,3R)- 1-(2-Chloro-4-(2- methyl-2H-pyrazolo [3,4-b]pyridin-5-yl) phenyl)-4-oxo- 5-azaspiro[2.4] heptan-5-yl)- N-(2-methyl-1-(5-(2- methylpyrimidin-5-yl)- 3-(trifluoromethyl)- 1H-pyrazol- 1-yl)propan-2-yl)-3-(1- (methyl-d₃)- 1H-pyrazol-4- yl)propanamide, isomer 1 | 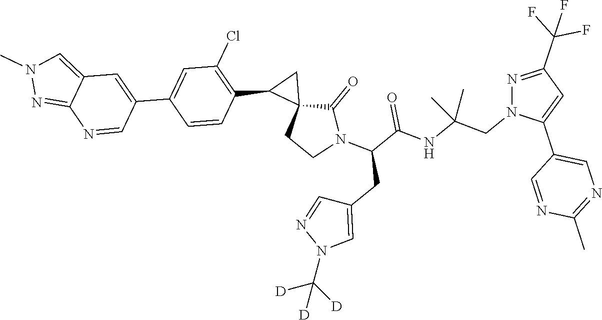 [SMILES: [2H]C([2H])([2H])n1cc(C[C@H](C(=O)NC(C)(C)Cn2nc(C(F)(F)F)cc2-c2cnc(C)nc2)N2CC[C@@]3(C[C@@H]3c3ccc(-c4cnc5nn(C)cc5c4)cc3Cl)C2=O)cn1] | 789.4 (M + H) |
| 115 | (2R)-N-(2- Methyl-1-(5-(2- methylpyrimidin-5-yl)- 3-(trifluoromethyl)- 1H-pyrazol- 1-yl)propan-2- yl)-2-((trans)- 1-(6-(2-methyl-2H- pyrazolo[3,4-b] pyridin-5-yl)- 2-(trifluoromethyl) pyridin-3- yl)-4-oxo-5- azaspiro[2.4] heptan-5-yl)-3- (1-(methyl-d₃)- 1H-pyrazol-4- yl)propanamide, isomer 1 | 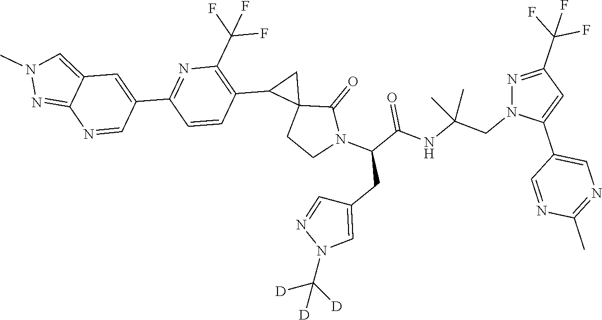 [SMILES: [2H]C([2H])([2H])n1cc(C[C@H](C(=O)NC(C)(C)Cn2nc(C(F)(F)F)cc2-c2cnc(C)nc2)N2CCC3(CC3c3ccc(-c4cnc5nn(C)cc5c4)nc3C(F)(F)F)C2=O)cn1] | 824.4 (M + H) |

### Example 116

### (2R)-2-(trans)-(1-(6-(4-Acetylpiperazin-1-yl)-2-(trifluoromethyl)pyridin-3-yl)-4-oxo-5-azaspiro[2.4]heptan-5-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide, Isomer 1

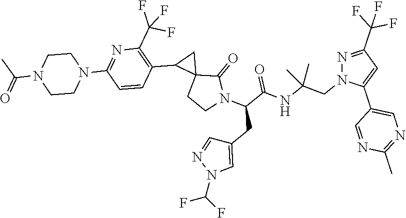 [SMILES: CC(=O)N1CCN(c2ccc(C3CC34CCN([C@H](Cc3cnn(C(F)F)c3)C(=O)NC(C)(C)Cn3nc(C(F)(F)F)cc3-c3cnc(C)nc3)C4=O)c(C(F)(F)F)n2)CC1]

A vial was charged with (R)-2-((trans)-1-(6-chloro-2-(trifluoromethyl)pyridin-3-yl)-4-oxo-5-azaspiro[2.4]heptan-5-yl)-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide (49.3 mg, 64.9 μmol) isomer 1,1-(piperazin-1-yl) ethan-1-one (60 mg, 7.2 Eq, 0.47 mmol), and DMSO (0.3 mL), flushed with Ar, sealed, and stirred at 100° C. for 27 h. The reaction mixture was cooled to RT, combined with a previous batch run on 15 μmol scale, and purified by reversed phase prep-HPLC (XSelect CSH C18, 30 mm×250 mm, 5 μm, 40 mL/min), eluting with a gradient of 5-95% ACN in 10 mM aq. NH₄OAc over 19 min, to provide the title compound (36.3 mg, 66%) as a white solid. ES-MS m/z 852.6 (M+H).

### Example 117

### (2R)-3-(1-(Difluoromethyl)-1H-pyrazol-4-yl)-2-((1S,3R)-1-(2-fluoro-4-(2-methyl-2H-pyrazolo[3,4-b]pyridin-5-yl)phenyl)-4-oxo-5-azaspiro[2.4]heptan-5-yl)-N-(2-methyl-1-(5-(1-methyl-6-oxo-1,6-dihydropyridin-3-yl)-3-(trifluoromethyl)-1H-pyrazol-1-yl)propan-2-yl)propanamide

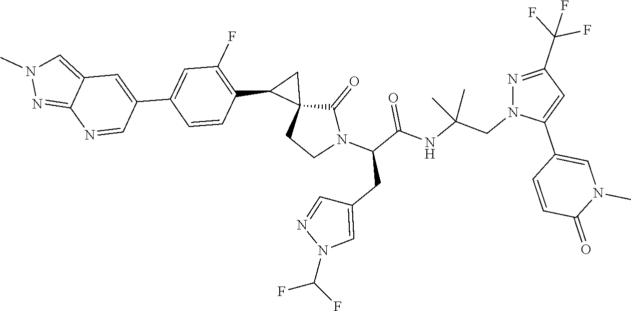

A mixture of (1R,2S)-1-(2-chloroethyl)-N—((R)-3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-2-(2-fluoro-4-(2-methyl-2H-pyrazolo[3,4-b]pyridin-5-yl)phenyl)cyclopropane-1-carboxamide (830 mg, 920 μmol) and Cs₂CO₃ (630 mg, 1.93 mmol) in NMP (8 mL) was stirred for 5 h at RT. The reaction mixture diluted with EtOAc (200 mL), washed with a mixture of water (200 mL) and sat. aq. NaCl (50 mL). Organic layer was isolated and aq. layer was extracted with EtOAc (60 mL). Then combined organic layers were washed with water (250 mL) twice and sat. aq. NaCl one time. The organic layer was isolated, dried over anhydrous sodium sulfate, filtered and concentrated to give light brown solid. The light brown solid was purified by reversed phase flash chromatography using a gradient of 40-60% ACN in 10 mM aq. NH₄HCO₃ to obtain the title compound (415 mg, 55%) as an off-white powder. ES-MS m/z 821.2 (M+H).

The compounds in the following table were prepared as described in Example 117. ACN is suitable replacements for NMP. Reaction times (1 h to overnight) may vary, and differing methods can be used to work up or purify the compounds (normal phase, high, neutral or low pH reversed phase). Such variances would be apparent to one skilled in the art.

TABLE 38

| Ex- | | | ES-MS |
|---|---|---|---|
| ample | Chemical Name | Structure | m/z |
| 118^{a} | (2R)-3-(1- (Difluoromethyl)- 1H-pyrazol-4-yl)- N-(1-(5-(5-fluoro- 1-methyl-6-oxo-1,6- dihydropyridin-3-yl)- 3-(trifluoromethyl)- 1H-pyrazol-1-yl)-2- methylpropan-2-yl)- 2-((1S,3R)-1- (2-fluoro-4-(2- methyl-2H- pyrazolo[3,4- b]pyridin-5-yl) phenyl)-4-oxo- 5-azaspiro[2.4] heptan-5- yl)propanethioamide, Isomer 1 | 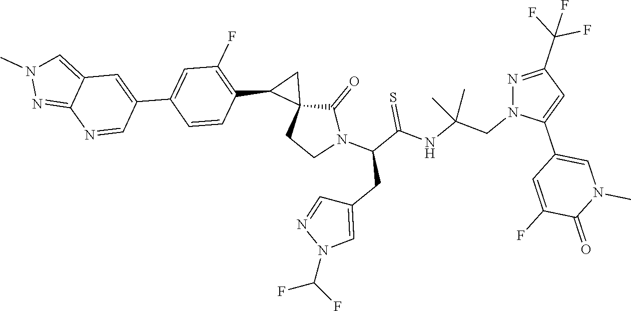 | 855.7 (M + H) |
| 119^{b} | (2R)-3-(1- (Difluoromethyl)- 1H-pyrazol-4-yl)- N-(1-((5-(5-fluoro- 1-methyl-6-oxo-1,6- dihydropyridin-3-yl)- 3-(trifluoromethyl)- 1H-pyrazol-1-yl) methyl)cyclopropyl)- 2-((1S,3R)-1- (2-fluoro-4-(2- methyl-2H- pyrazolo[3,4- b]pyridin-5-yl) phenyl)-4-oxo- 5-azaspiro[2.4] heptan-5- yl)propanamide, Isomer 1 | 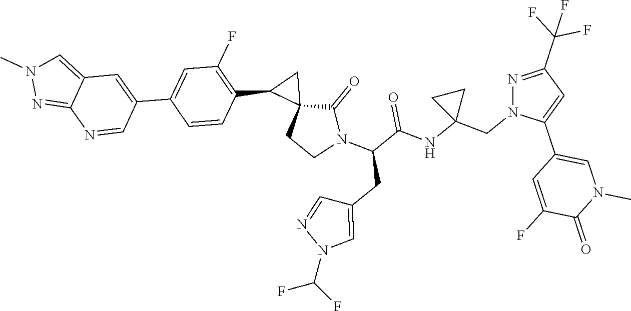 | 837.0 (M + H) |

^{a}ACN, 1 h, diluted with ACN, filtered and concentrated under reduced pressure, then purified by silica gel chromatography using a gradient of 1 to 10% 2M ammonia in MeOH in DCM, then repurified by reversed phase prep-HPLC (Kinetex ® EVO C18, 30 × 100 mm, 5 μm, 85 mL/min, 2 injections) using a gradient of 33-67% ACN in 10 mM aq. NH₄HCO₃ (with 5% MeOH) over 8 min;
^{b}ACN, 3.5 h, diluted with ACN, filtered and concentrated under reduced pressure, then purified by reversed flash chromatography eluting with 40-65% ACN in 10 mM aq. NH₄HCO₃(with 5% MeOH)

### Example 120

### (R)-3-(1-(Difluoromethyl)-1H-pyrazol-4-yl)-N-(2-methyl-1-(5-(1-(methyl-d3)-6-oxo-1,6-dihydropyridin-3-yl)-3-(trifluoromethyl)-1H-pyrazol-1-yl)propan-2-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide

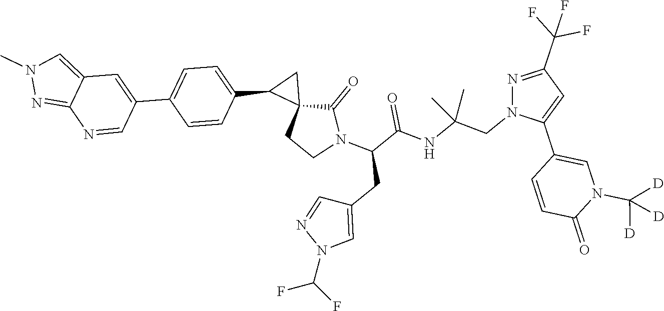 [SMILES: [2H]C([2H])([2H])n1cc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nn(C)cc5c4)cc3)C2=O)ccc1=O]

To a solution of 5-(1-(2-amino-2-methylpropyl)-3-(trifluoromethyl)-1H-pyrazol-5-yl)-1-(methyl-d3)pyridin-2(1H)-one hydrochloride (64 mg, 70 wt. %, 0.13 mmol) and (R)-3-(1-(difluoromethyl)-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanoic acid (64 mg, 0.13 mmol) in anhydrous DMA (1.3 mL) was added 4-methylmorpholine (70 μL, 0.64 mmol). The solution was cooled to 0° C. in an ice-water bath, and DEPBT (76 mg, 0.25 mmol) was added. The reaction mixture was stirred at 0° C. then the ice bath was allowed to expire over 18 h. The reaction mixture was directly purified by reversed phase prep-HPLC (XSelect CSH C18 column, 30 mm×150 mm, 5 μm, 40 mL/min) using a gradient of 5-95% ACN in 10 mM aq. NH₄OAc, pH=10 over 19 min to obtain the title compound (60 mg, 59%) as a white powder. ES-MS m/z 806.4 (M+H).

The compounds in the following table were prepared as described in Example 120 using the appropriate carboxylic acid and the appropriate amine. Reactants can be added in different orders or in differing equivalency. Differing methods can be used to work up or purify the compounds (normal phase, or high, neutral or low pH reversed phase). Such variances would be apparent to one skilled in the art.

TABLE 39

| Ex- | | | ES-MS |
|---|---|---|---|
| ample | Chemical Name | Structure | m/z |
| 121^{a} | (R)-N-(1-(5-(6- (Difluoromethoxy)pyridazin-3- yl)-3-(trifluoromethyl)-1H- pyrazol-1-yl)-2-methylpropan-2- yl)-3-(1-(difluoromethyl)-1H- pyrazol-4-yl)-2-((1R,3R)-1-(4- (2-methyl-2H-pyrazolo[3,4- b]pyridin-5-yl)phenyl)-4-oxo-5- azaspiro[2.4]heptan-5- yl)propanamide | 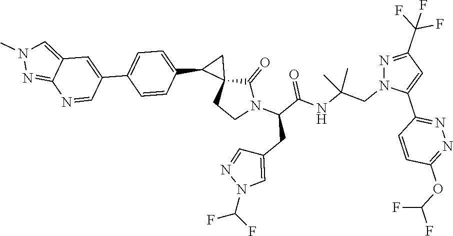 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccc(OC(F)F)nn4)C5=O)cc3)cnc2n1] | ES-MS m/z 840.4 (M + H) |
| 122^{a} | (R)-N-(1-(5-(2-Cyclopropyl-4- hydroxy-6-methylpyrimidin-5- yl)-3-(trifluoromethyl)-1H- pyrazol-1-yl)-2-methylpropan-2- yl)-3-(1-(difluoromethyl)-1H- pyrazol-4-yl)-2-((1R,3R)-1-(4- (2-methyl-2H-pyrazolo[3,4- b]pyridin-5-yl)phenyl)-4-oxo-5- azaspiro[2.4]heptan-5- yl)propanamide | 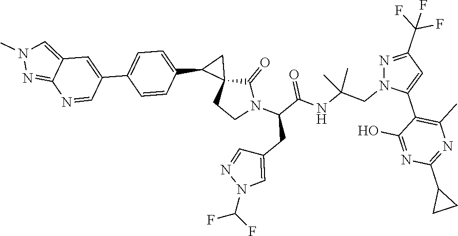 [SMILES: Cc1nc(C2CC2)nc(O)c1-c1cc(C(F)(F)F)nn1CC(C)(C)NC(=O)[C@@H](Cc1cnn(C(F)F)c1)N1CC[C@@]2(C[C@@H]2c2ccc(-c3cnc4nn(C)cc4c3)cc2)C1=O] | ES-MS m/z 844.4 (M + H) |
| 123^{b} | (R)-N-(1-(5-(1-(Cyanomethyl)- 2-oxo-1,2-dihydropyridin-3-yl)- 3-(trifluoromethyl)-1H-pyrazol- 1-yl)-2-methylpropan-2-yl)-3-(1- (difluoromethyl)-1H-pyrazol-4- yl)-2-((1R,3R)-1-(4-(2-methyl- 2H-pyrazolo[3,4-b]pyridin-5- yl)phenyl)-4-oxo-5- azaspiro[2.4]heptan-5- yl)propanamide | 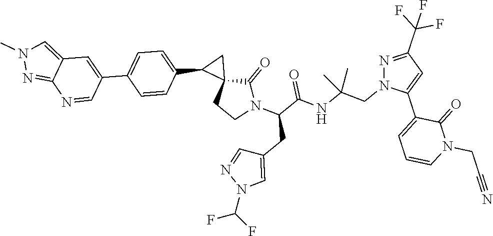 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4cccn(CC#N)c4=O)C5=O)cc3)cnc2n1] | ES-MS m/z 828.4 (M + H) |
| 124^{a} | (R)-N-(1-(5-(2,5-Difluoro-4- (hydroxymethyl)phenyl)-3- (trifluoromethyl)-1H-pyrazol-1- yl)-2-methylpropan-2-yl)-3-(1- (difluoromethyl)-1H-pyrazol-4- yl)-2-((1R,3R)-1-(4-(2-methyl- 2H-pyrazolo[3,4-b]pyridin-5- yl)phenyl)-4-oxo-5- azaspiro[2.4]heptan-5- yl)propanamide | 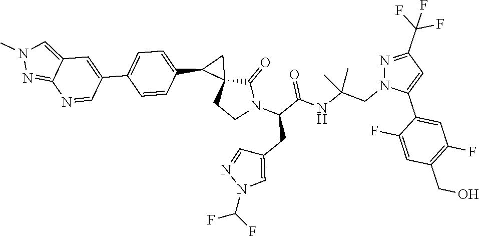 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4cc(F)c(CO)cc4F)C5=O)cc3)cnc2n1] | ES-MS m/z 838.3 (M + H) |
| 125^{a} | (R)-3-(1-(Difluoromethyl)-1H- pyrazol-4-yl)-N-(2-methyl-1-(5- (6-oxo-1-(2,2,2-trifluoroethyl)- 1,6-dihydropyridin-3-yl)-3- (trifluoromethyl)-1H-pyrazol-1- yl)propan-2-yl)-2-((1R,3R)-1-(4- (2-methyl-2H-pyrazolo[3,4- b]pyridin-5-yl)phenyl)-4-oxo-5- azaspiro[2.4]heptan-5- yl)propanamide | 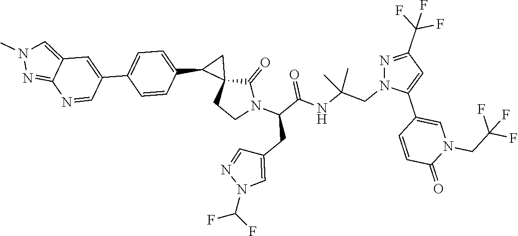 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccc(=O)n(CC(F)(F)F)c4)C5=O)cc3)cnc2n1] | ES-MS m/z 871.4 (M + H) |
| 126^{b} | (R)-N-(1-(5-(1-(2-Amino-2- oxoethyl)-2-oxo-1,2- dihydropyridin-3-yl)-3- (trifluoromethyl)-1H-pyrazol-1- yl)-2-methylpropan-2-yl)-3-(1- (difluoromethyl)-1H-pyrazol-4- yl)-2-((1R,3R)-1-(4-(2-methyl- 2H-pyrazolo[3,4-b]pyridin-5- yl)phenyl)-4-oxo-5- azaspiro[2.4]heptan-5- yl)propanamide | 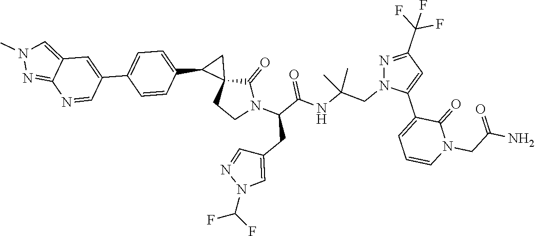 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4cccn(CC(N)=O)c4=O)C5=O)cc3)cnc2n1] | ES-MS m/z 846.4 (M + H) |
| 127^{a} | (R)-3-(1-(Difluoromethyl)-1H- pyrazol-4-yl)-N-(2-methyl-1-(1′- (2-(methylamino)-2-oxoethyl)-5- (trifluoromethyl)-1′H,2H-[3,4′- bipyrazol]-2-yl)propan-2-yl)-2- ((1R,3R)-1-(4-(2-methyl-2H- pyrazolo[3,4-b]pyridin-5- yl)phenyl)-4-oxo-5- azaspiro[2.4]heptan-5- yl)propanamide | 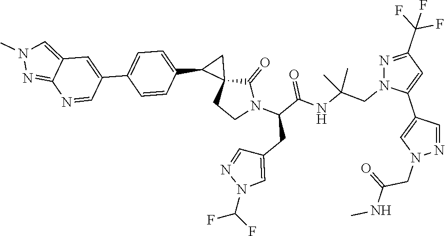 [SMILES: CNC(=O)Cn1cc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nn(C)cc5c4)cc3)C2=O)cn1] | ES-MS m/z 833.4 (M + H) |
| 128^{a} | (R)-N-(1-(5-(5-Cyano-1,4- dimethyl-6-oxo-1,6- dihydropyridin-3-yl)-3- (trifluoromethyl)-1H-pyrazol-1- yl)-2-methylpropan-2-yl)-3-(1- (difluoromethyl)-1H-pyrazol-4- yl)-2-((1R,3R)-1-(4-(2-methyl- 2H-pyrazolo[3,4-b]pyridin-5- yl)phenyl)-4-oxo-5- azaspiro[2.4]heptan-5- yl)propanamide | 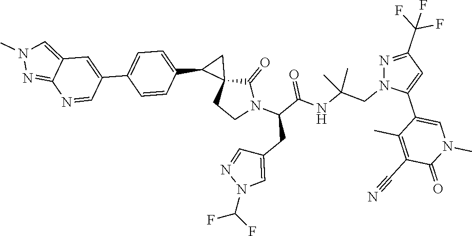 | ES-MS m/z 842.3 (M + H) |
| 129^{a} | (R)-3-(1-(Difluoromethyl)-1H- pyrazol-4-yl)-N-(2-methyl-1-(5- (2-methyl-1-oxo-1,2- dihydroisoquinolin-4-yl)-3- (trifluoromethyl)-1H-pyrazol-1- yl)propan-2-yl)-2-((1R,3R)-1-(4- (2-methyl-2H-pyrazolo[3,4- b]pyridin-5-yl)phenyl)-4-oxo-5- azaspiro[2.4]heptan-5- yl)propanamide | 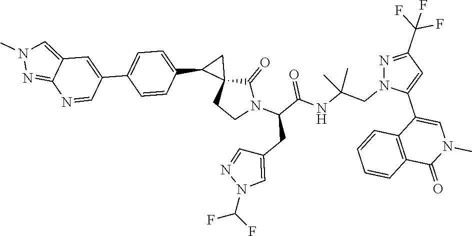 | ES-MS m/z 853.4 (M + H) |
| 130^{b} | (R)-3-(1-(Difluoromethyl)-1H- pyrazol-4-yl)-N-(2-methyl-1-(5- (1-methyl-6-oxo-5- (trifluoromethyl)-1,6- dihydropyridin-3-yl)-3- (trifluoromethyl)-1H-pyrazol-1- yl)propan-2-yl)-2-((1R,3R)-1-(4- (2-methyl-2H-pyrazolo[3,4- b]pyridin-5-yl)phenyl)-4-oxo-5- azaspiro[2.4]heptan-5- yl)propanamide | 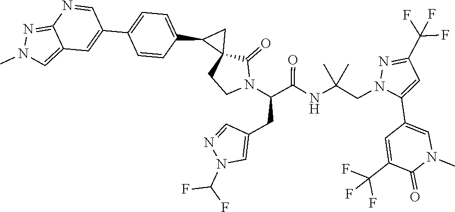 | ES-MS m/z 871.4 (M + H) |
| 131^{c} | (R)-3-(1-(Difluoromethyl)-1H- pyrazol-4-yl)-N-(1-(5-(8- fluoroimidazo[1,2-a]pyridin-6- yl)-3-(trifluoromethyl)-1H- pyrazol-1-yl)-2-methylpropan-2- yl)-2-((1R,3R)-1-(4-(2-methyl- 2H-pyrazolo[3,4-b]pyridin-5- yl)phenyl)-4-oxo-5- azaspiro[2.4]heptan-5- yl)propanamide | 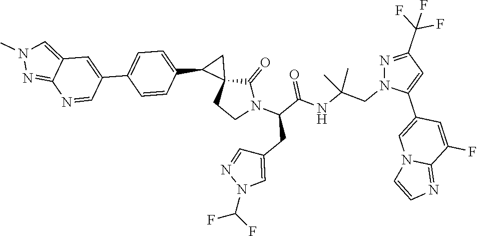 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4cc(F)c6nccn6c4)C5=O)cc3)cnc2n1] | ES-MS m/z 830.2 (M + H) |
| 132^{c} | 5-(1-(2-((R)-3-(1- (Difluoromethyl)-1H-pyrazol-4- yl)-2-((1R,3R)-1-(4-(2-methyl- 2H-pyrazolo[3,4-b]pyridin-5- yl)phenyl)-4-oxo-5- azaspiro[2.4]heptan-5- yl)propanamido)-2- methylpropyl)-3- (trifluoromethyl)-1H-pyrazol-5- yl)-6-oxo-1,6-dihydropyridine-2- carboxamide | 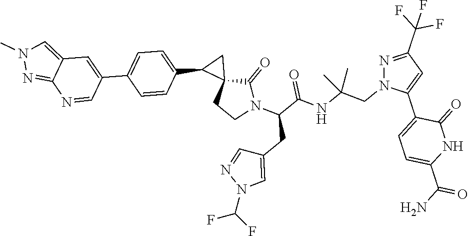 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccc(C(N)=O)[nH]c4=O)C5=O)cc3)cnc2n1] | ES-MS m/z 832.2 (M + H) |
| 133^{e} | (R)-3-(1-(Difluoromethyl)-1H- pyrazol-4-yl)-N-(2-methyl-1-(5- (5-methyl-4-oxo-4,5- dihydropyrazolo[1,5-a]pyrazin- 7-yl)-3-(trifluoromethyl)-1H- pyrazol-1-yl)propan-2-yl)-2- ((1R,3R)-1-(4-(2-methyl-2H- pyrazolo[3,4-b]pyridin-5- yl)phenyl)-4-oxo-5- azaspiro[2.4]heptan-5- yl)propanamide | 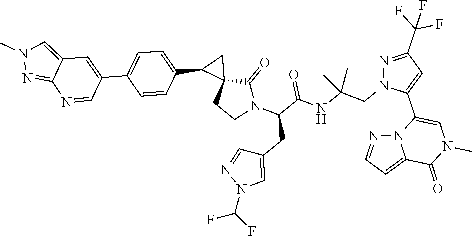 | ES-MS m/z 861.2 (M + H) |
| 134^{d} | (R)-3-(1-(Difluoromethyl)-1H- pyrazol-4-yl)-N-(1-(5-(1- (difluoromethyl)-2-oxo-1,2- dihydropyridin-4-yl)-3- (trifluoromethyl)-1H-pyrazol-1- yl)-2-methylpropan-2-yl)-2- ((1S,3R)-1-(2-fluoro-4-(2- methyl-2H-pyrazolo[3,4- b]pyridin-5-yl)phenyl)-4-oxo-5- azaspiro[2.4]heptan-5- yl)propanamide | 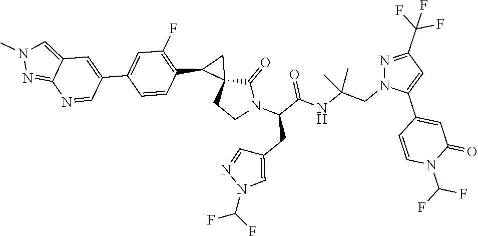 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccn(C(F)F)c(=O)c4)C5=O)c(F)c3)cnc2n1] | 857.4 (M + H) |
| 135^{d} | (R)-3-(1-(Difluoromethyl)-1H- pyrazol-4-yl)-N-(1-(5-(6- ((dimethylamino)methyl)pyridin- 3-yl)-3-(trifluoromethyl)-1H- pyrazol-1-yl)-2-methylpropan-2- yl)-2-((1S,3R)-1-(2-fluoro-4-(2- methyl-2H-pyrazolo[3,4- b]pyridin-5-yl)phenyl)-4-oxo-5- azaspiro[2.4]heptan-5- yl)propanamide | 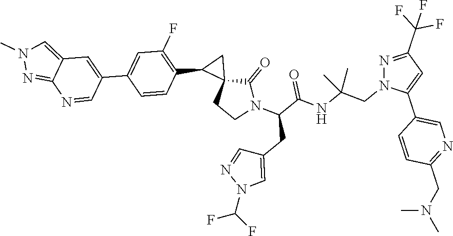 [SMILES: CN(C)Cc1ccc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nn(C)cc5c4)cc3F)C2=O)cn1] | 848.4 (M + H) |

^{a}purified by reverse phase using an Phenomenex Kinetex EVO (C18 column 30 × 250 mm, 5 μm, 40 mL/min) using ACN and water with gradient of 5 to 95% with 10 mM NH₄HCO₃ modifier;
^{b}purified by reverse phase using Phenomenex Kinetex EVO (C18, 30 mm × 100 mm, 5.0 μm, 20 mL/min), using ACN and water with gradient of 5 to 95% with 0.1% FA;
^{c}purified by reverse phase XBridge (C18, 19 × 150 mm, 5 μm, 40 mL/min) using ACN and water with gradient of 30 to 60% with 20 mM aq. NH₄HCO₃
^{d}purified by reverse phase using Phenomenex Kinetex EVO (C18, 30 × 100 mm, 5 μm) eluting with a gradient of ACN in 10 mM aq. NH₄HCO₃, pH = 10 (with 5% MeOH);
^{e}purified by silica gel chromatography using a gradient of 0-13% MeOH in DCM, then purified again by reverse phase YMC Triart (C18, 25 × 150 mm, 5 μm, 25 mL/min) using ACN and water with gradient of 36 to 66% with 20 mM aq. NH₄HCO₃.

### Example 136

### (1S,2S)-N—((R)-3-(1-(Difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)-N-(methyl-d₃)-2-(6-(2-methyl-2H-pyrazolo[3,4-b]pyridin-5-yl)-2-(trifluoromethyl)pyridin-3-yl)cyclopropane-1-carboxamide

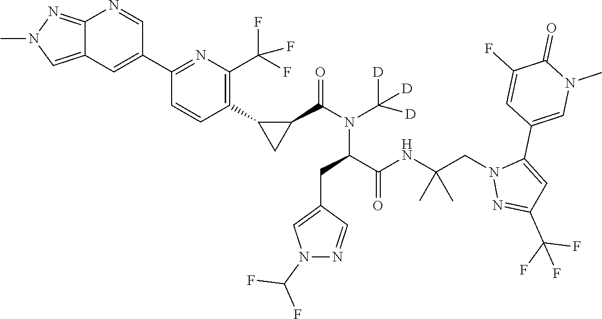

A 1 dram vial was charged with (1S,2S)-2-(6-(2-methyl-2H-pyrazolo[3,4-b]pyridin-5-yl)-2-(trifluoromethyl)pyridin-3-yl)cyclopropane-1-carboxylic acid (36.3 mg, 100 μmol), (R)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-((methyl-d₃)amino)propanamide hydrochloride (55 mg, 96 μmol), a stirbar, and DMSO (1 mL). The mixture was stirred at RT until homogeneous, then TEA (50 μL, 0.36 mmol) was added, followed by HATU (52 mg, 0.14 mmol). The reaction mixture was stirred at RT under argon for 3 h, quenched with water (0.3 mL), and filtered. The filtrate was purified by reversed phase prep-HPLC (XSelect CSH C18, 50×250 mm, 5 μm, 100 mL/min), eluting with a gradient of 5-95% ACN in 10 mM aq. NH₄OAc, to provide the title compound (10.3 mg, 11%) as a white solid. ES-MS m/z 881.4 (M+H).

The compounds in the following table were prepared as described in Example 136 using the appropriate carboxylic acid or Li carboxylate salt and the appropriate amine or amine salt. Reactants can be added in different orders or in differing equivalency. DMA is a suitable replacement for DMF. Reaction times may vary and differing methods can be used to work up or purify the compounds (normal phase, or high, neutral or low pH reversed phase). Such variances would be apparent to one skilled in the art.

TABLE 40

| Ex- | | | ES-MS |
|---|---|---|---|
| ample | Chemical Name | Structure | m/z |
| 137 | (1S,2S)-N-((R)-3-(1- (Difluoromethyl)-1H- pyrazol-4-yl)-1-((1-(5-(5- fluoro-1-methyl-6-oxo-1,6- dihydropyridin-3-yl)-3- (trifluoromethyl)-1H- pyrazol-1-yl)-2- methylpropan-2-yl)amino)- 1-oxopropan-2-yl)-2-(2- (difluoromethyl)-6-(2- methyl-2H-pyrazolo[3,4- b]pyridin-5-yl)pyridin-3- yl)-N-(methyl- d₃)cyclopropane-1- carboxamide | 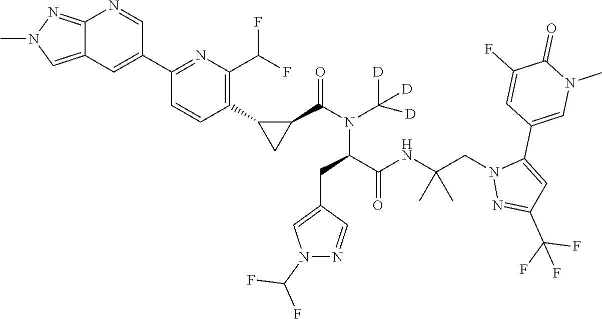 | 863.4 (M + H) |
| 138^{a} | (1S,2S)-N-((S)-3-(1- (Difluoromethyl)-1H- pyrazol-4-yl)-1-((1-(5-(5- fluoro-1-methyl-6-oxo-1,6- dihydropyridin-3-yl)-3- (trifluoromethyl)-1H- pyrazol-1-yl)-2- methylpropan-2-yl)amino)- 1-oxopropan-2-yl)-2-(2- (difluoromethyl)-6-(2- methyl-2H-pyrazolo[3,4- b]pyridin-5-yl)pyridin-3- yl)-N-(methyl- d₃)cyclopropane-1- carboxamide | 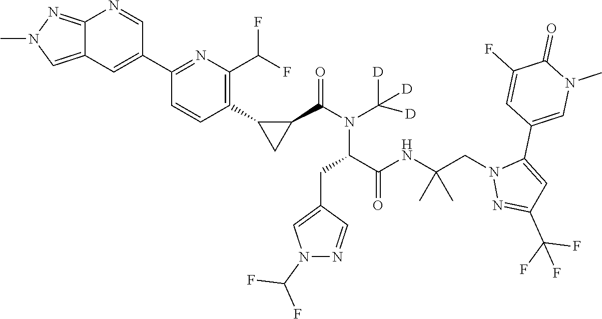 | 863.4 (M + H) |
| 139 | (1S,2S)-N-((R)-3-(1- (Difluoromethyl)-1H- pyrazol-4-yl)-1-((1-(5-(5- fluoro-1-methyl-6-oxo-1,6- dihydropyridin-3-yl)-3- (trifluoromethyl)-1H- pyrazol-1-yl)-2- methylpropan-2-yl)amino)- 1-oxopropan-2-yl)-N- (methyl-d₃)-2-(2-(2- methyl-2H-pyrazolo[3,4- b]pyridin-5-yl)-4- (trifluoromethyl)pyrimidin- 5-yl)cyclopropane-1- carboxamide | 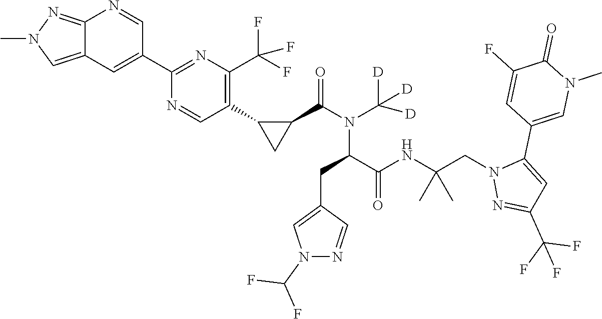 | 882.4 (M + H) |
| 140^{b} | (trans)-N-((R)-3-(1- (difluoromethyl)-1H- pyrazol-4-yl)-1-((1-(5-(5- fluoro-1-methyl-6-oxo-1,6- dihydropyridin-3-yl)-3- (trifluoromethyl)-1H- pyrazol-1-yl)-2- methylpropan-2-yl)amino)- 1-oxopropan-2-yl)-2-(2- fluoro-4-(2-methyl-2H- pyrazolo[3,4-b]pyridin-5- yl)phenyl)-2-methyl-N- (methyl-d₃)cyclopropane-1- carboxamide, isomer 1 | 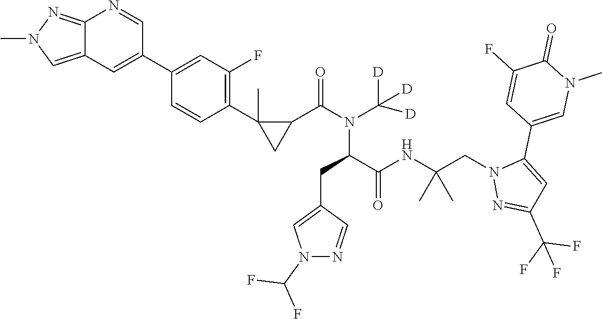 | 844.4 (M + H) |

aExample 138 was isolated as minor product due to epimerization happened during preparation of example 137;
^{b}First-eluting diastereomer when purified by reversed phase prep-HPLC (XSelect CSH C18, 50 mm × 250 mm, 5 μm, 100 mL/min), eluting with a gradient of 5-95% ACN in 0.1% aq. FA.

### Example 141

(R)-3-(1-(Difluoromethyl)-1H-pyrazol-4-yl)-N-(1-(5-(5-fluoro-1-(methyl-d3)-6-oxo-1,6-dihydropyridin-3-yl)-3-(trifluoromethyl)-1H-pyrazol-1-yl)-2-methylpropan-2-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide

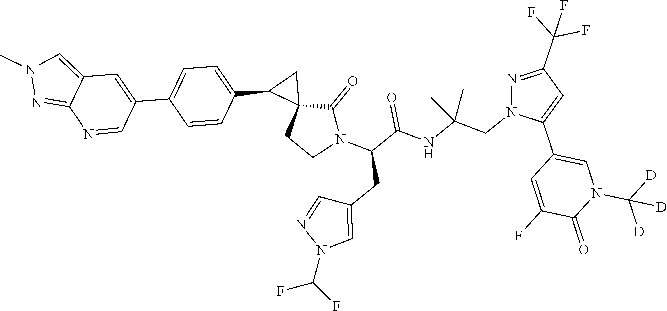 [SMILES: [2H]C([2H])([2H])n1cc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nn(C)cc5c4)cc3)C2=O)cc(F)c1=O]

A vial was charged with 3-fluoro-1-(methyl-d3)-5-(4,4,5,5-tetramethyl-1,3,2-dioxaborolan-2-yl)pyridin-2(1H)-one (34 mg, 0.13 mmol), [1,1′-Bis(diphenylphosphino)ferrocene]dichloropalladium (II) CH₂Cl₂ complex (16 mg, 20 μmol), K₂CO₃ (42 mg, 0.30 mmol), and a solution of (R)—N-(1-(5-bromo-3-(trifluoromethyl)-1H-pyrazol-1-yl)-2-methylpropan-2-yl)-3-(1-(difluoromethyl)-1H-pyrazol-4-yl)-2-((1R,3R)-1-(4-(2-methyl-2H-pyrazolo[3,4-b]pyridin-5-yl)phenyl)-4-oxo-5-azaspiro[2.4]heptan-5-yl)propanamide (70 mg, 90 μmol) in 1,4-dioxane (700 μL). Water (200 μL) was added and the reaction mixture was sparged with argon while gradually heating to 80° C. over 5 min. Upon reaching 80° C., sparging was stopped and the reaction mixture was stirred under argon for 2.25 h. The reaction mixture was then cooled to RT and treated with SiliaMetS Thiol resin (Silicycle, 1.46 mmol/g, 150 mg), diluted with ACN (1 mL), and stirred at RT overnight. The reaction mixture was then filtered and purified by reversed phase pre-HPLC (XSelect CSH C18, 30 mm×150 mm, 5 μm, 40 mL/min), eluting with a gradient of 5-95% ACN in 0.1% aq. FA, to provide the title compound (37 mg, 49%) as a fluffy white solid. ES-MS m/z 824.4 (M+H).

The compounds in the following table were prepared as described in Example 141 using the appropriate boron ester or boronic acid and the appropriate aryl bromide coupling partners. Reactants can be added in different orders or in differing equivalency. Cs₂CO₃ is a suitable replacement of K₂CO₃. Reaction times may vary, and differing methods can be used to work up or purify the compounds (normal phase, high or low pH prep-HPLC). Such variances would be apparent to one skilled in the art.

TABLE 41

| Ex- | | | ES-MS |
|---|---|---|---|
| ample | Chemical Name | Structure | m/z |
| 142^{a} | (R)-3-(1-(Difluoro- methyl)-1H- pyrazol-4-yl)- N-(2-methyl-1-(5- (2-(4-oxopiperidin-1- yl)pyrimidin-5-yl)-3- (trifluoromethyl)- 1H-pyrazol-1- yl)propan-2-yl)-2- ((1R,3R)-1-(4- (2-methyl-2H- pyrazolo[3,4- b]pyridin-5-yl) phenyl)-4-oxo-5- azaspiro[2.4] heptan-5- yl)propanamide | 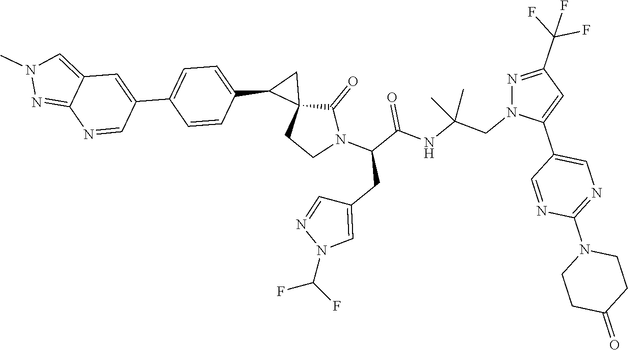 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4cnc(N6CCC(=O)CC6)nc4)C5=O)cc3)cnc2n1] | 871.6 (M + H) |
| 143^{a} | (R)-N-(1-(5-(6- Cyanopyridin-3- yl)-3-(trifluoro- methyl)-1H- pyrazol-1-yl)-2- methylpropan-2- yl)-3-(1-(difluoro- methyl)-1H- pyrazol-4-yl)-2- ((1R,3R)-1-(4-(2- methyl-2H- pyrazolo[3,4- b]pyridin-5-yl) phenyl)-4-oxo-5- azaspiro[2.4] heptan-5- yl)propanamide | 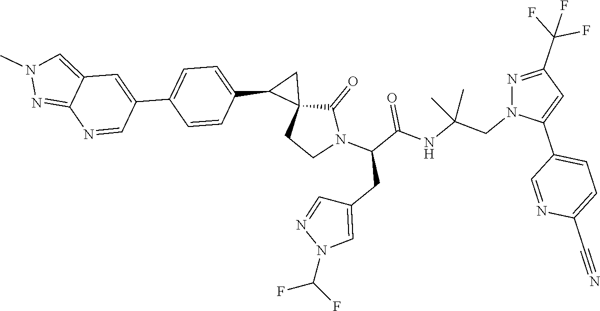 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccc(C#N)nc4)C5=O)cc3)cnc2n1] | 798.6 (M + H) |

^{a}Cs₂CO₃, 1 h. Purified by reverse phase using an XSelect CSH C18 column 30 × 250 mm, 5 μm, 40 mL/min gradient of 5 to 95% ACN in 10 mM aq. NH₄OAc.

### Example 144

### (R)-3-(1-(Difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-((1R,3R)-1-(2-methyl-4-(2-methylimidazo[1,2-a]pyrimidin-6-yl)phenyl)-4-oxo-5-azaspiro[2.4]heptan-5-yl)propanamide

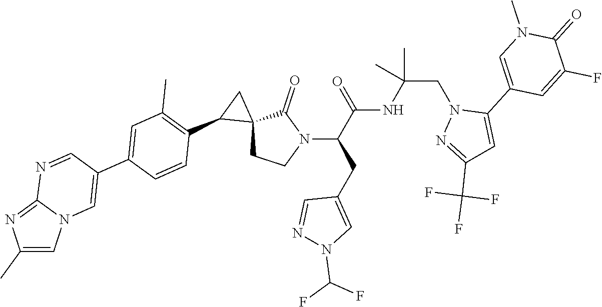 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4cc(F)c(=O)n(C)c4)C5=O)c(C)c3)cnc2n1]

A mixture of (R)-2-((1R,3R)-1-(4-bromo-2-methylphenyl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (786.8 mg, 814 μmol), (2-methylimidazo[1,2-a]pyrimidin-6-yl) boronic acid (289.2 mg, 1.63 mmol), Cs₂CO₃ (797.5 mg, 2.45 mmol), and Pd(dppf)Cl₂ (DCM adduct, 99.6 mg, 122 μmol) was placed under argon. Then, argon-sparged 1,4-dioxane (6.0 mL) and H₂O (2.0 mL) were added, and the solution was heated to 90° C. After 2 h, the reaction was removed from heating and quenched by dilution with H₂O and EtOAc. The organic layer was removed, and the aq. layer was extracted with EtOAc (4×). The combined organic layers were then dried over Na₂SO₄, filtered, concentrated under reduced pressure. The residue was purified on silica gel eluting with 20-100% 3:1 EtOAc:EtOH in heptane, followed by prep-HPLC (CSH C18, 2.1 mm×50 mm, 1.7 μm) eluting with 5-95% ACN in H₂O (+0.1% HCO₂H) to obtain the title compound (504.5 mg, 70% yield) as a tan solid. ES-MS m/z 835 (M+H).

### Example 145

### (R)—N-(1-(5-(1-Cyclopropyl-2-oxo-1,2-dihydropyridin-4-yl)-3-(trifluoromethyl)-1H-pyrazol-1-yl)-2-methylpropan-2-yl)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)propanamide

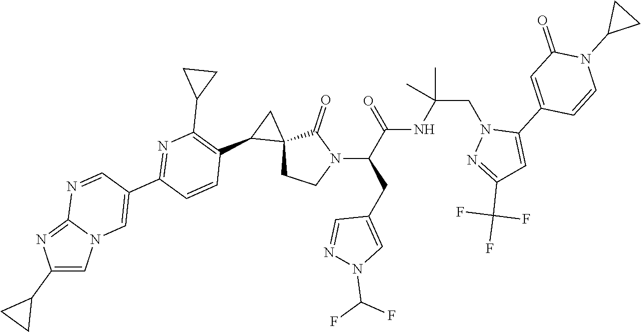 [SMILES: CC(C)(Cn1nc(C(F)(F)F)cc1-c1ccn(C2CC2)c(=O)c1)NC(=O)[C@@H](Cc1cnn(C(F)F)c1)N1CC[C@@]2(C[C@@H]2c2ccc(-c3cnc4nc(C5CC5)cn4c3)nc2C2CC2)C1=O]

To a mixture of (2-cyclopropylimidazo[1,2-a]pyrimidin-6-yl) boronic acid (0.51 g, 2.5 mmol) in 1,4-dioxane (15 mL) was added (R)-2-((1R,3R)-1-(6-chloro-2-methylpyridin-3-yl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide (1.23 g, 1.50 mmol), Cs₂CO₃ (1.62 g, 4.97 mmol), Pd(dppf)Cl₂ (135.4 mg, 165.8 μmol) and water (3.0 mL). The mixture was purged with N₂ for 3 min and heated at 90° C. for 1.5 h. The reaction mixture was cooled to RT and the aqueous layer was separated using pipette. The aqueous layer was then extracted with EtOAc (5 mL×2). The combined organic layers were then filtered over diatomaceous earth and diluted with 25 mL H₂O. The aqueous layer was separated and then extracted with EtOAc (2 mL×3). The combined organic layers were dried over Na₂SO₄, filtered and concentrated under reduced pressure. To the residue was added 1 g SiliaMetS® metal scavenger resin and stirred at RT for 30 min. The mixture was then filtered, and the residue was extracted with EtOAc, and the filtrate was then concentrated under reduced pressure. The residue was dissolved in DMF (6 mL), filtered by syringe filter, and purified by prep-HPLC (XSelect CSH C18, 40 mm×250 mm, 5 μm) using a gradient of 5-95% ACN in water (0.1% FA) to obtain the title compound (711.6 mg, 53%) as an off-white powder. ES-MS m/z 870 (M+H).

### Example 146

### (2R)-2-((1S,3R)-1-(2-Chloro-4-(2-cyclopropylimidazo[1,2-a]pyrimidin-6-yl)phenyl)-4-oxo-5-azaspiro[2.4]heptan-5-yl)-3-(1-(difluoromethyl)-1H-pyrazol-4-yl)-N-(2-methyl-1-(1-(1-methyl-2-oxopyrrolidin-3-yl)-4-(trifluoromethyl)-1H-imidazol-2-yl)propan-2-yl)propanamide

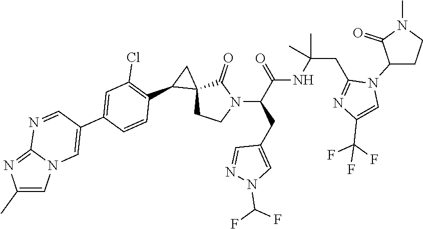 [SMILES: Cc1cn2cc(-c3ccc([C@H]4C[C@@]45CCN([C@H](Cc4cnn(C(F)F)c4)C(=O)NC(C)(C)Cc4nc(C(F)(F)F)cn4C4CCN(C)C4=O)C5=O)c(Cl)c3)cnc2n1]

A mixture of (2R)-2-((1S,3R)-1-(4-bromo-2-chlorophenyl)-4-oxo-5-azaspiro[2.4]heptan-5-yl)-3-(1-(difluoromethyl)-1H-pyrazol-4-yl)-N-(2-methyl-1-(1-(1-methyl-2-oxopyrrolidin-3-yl)-4-(trifluoromethyl)-1H-imidazol-2-yl)propan-2-yl)propanamide (160 mg, 186 μmol) in 1,4-dioxane (1 mL) was added (2-cyclopropylimidazo[1,2-a]pyrimidin-6-yl) boronic acid (52.8 mg, 260 μmol), Cs₂CO₃ (168.8 mg, 518.1 μmol), Pd(dppf)Cl₂ (19.8 mg, 24.2 μmol) and water (0.5 mL). The mixture was purged with N₂ for 3 min and heated at 90° C. for 1 h. The reaction mixture was cooled to RT, and 300 mg SiliaMetS® metal scavenger resin was added and stirred at RT for 30 min. The mixture was then filtered, and the residue was extracted with EtOAc, and the filtrate was then concentrated under reduced pressure. The residue was dissolved in DMF (3 mL), filtered by syringe filter, and purified by prep-HPLC (XSelect CSH C18, 50 mm×250 mm, 5 μm) using a gradient of 5-95% ACN in water (+0.1% FA), followed by chiral SFC separation (Chiralpak IC 30×250 mm, 5 μm), eluting with 10 mM aq. NH₄HCO₃ in MeOH to afford the title compound (27.4 mg, 17%) as the first eluting isomer. ES-MS m/z 853 (M+H).

### Example 147

### (1S,2S)-N—((R)-3-(1-(Difluoromethyl)-1H-pyrazol-4-yl)-1-((2-methyl-1-(5-(2-methylpyrimidin-5-yl)-3-(trifluoromethyl)-1H-pyrazol-1-yl)propan-2-yl)amino)-1-oxopropan-2-yl)-2-(6-(2-methyl-2H-pyrazolo[3,4-b]pyridin-5-yl)-2-(trifluoromethyl)pyridin-3-yl)cyclopropane-1-carboxamide

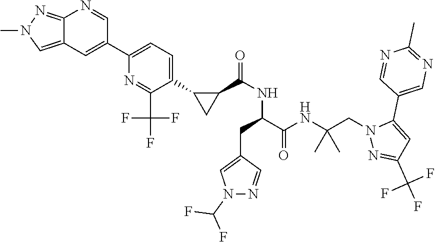 [SMILES: Cc1ncc(-c2cc(C(F)(F)F)nn2CC(C)(C)NC(=O)[C@@H](Cc2cnn(C(F)F)c2)NC(=O)[C@H]2C[C@@H]2c2ccc(-c3cnc4nn(C)cc4c3)nc2C(F)(F)F)cn1]

To a mixture of (1S,2S)-2-(6-(2-methyl-2H-pyrazolo[3,4-b]pyridin-5-yl)-2-(trifluoromethyl)pyridin-3-yl)cyclopropane-1-carboxylic acid (1.39 g, 3.85 mmol) and (R)-2-amino-3-(1-(difluoromethyl)-1H-pyrazol-4-yl)-N-(2-methyl-1-(5-(2-methylpyrimidin-5-yl)-3-(trifluoromethyl)-1H-pyrazol-1-yl)propan-2-yl)propanamide hydrochloride (2.2 g, 88% Wt, 3.77 mmol) was added HATU (3.1 g, 8.16 mmol), DIEA (2.0 mL, 11 mmol) and DMF (21 mL). The resulting mixture was stirred at 0° C. for 10 min, then allowed to warm to RT overnight. The crude mixture was filtered through a 0.45 μm syringe filter and concentrated under a stream of nitrogen. The crude product was purified by prep-HPLC (CSH XSelect, C18, 50 mm×250 mm, 5 μm) using a gradient of 5-95% ACN and water (with 0.1% FA) to obtain the title compound (1.09 g, 33%) as a white solid. ES-MS m/z 831 (M+H).

### Example 148

### (R)-2-((1S,3R)-1-(2-Chloro-4-(2-methylimidazo[1,2-a]pyrimidin-6-yl)phenyl)-4-oxo-5-azaspiro[2.4]heptan-5-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide

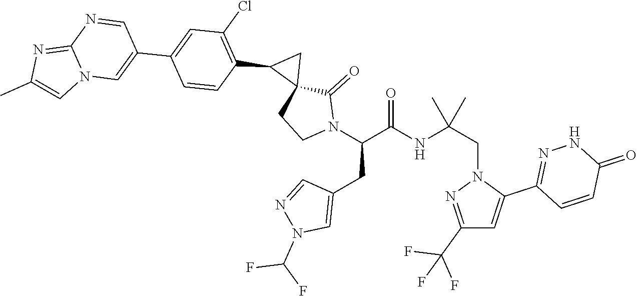 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4ccc(=O)[nH]n4)C5=O)c(Cl)c3)cnc2n1]

To a solution of (R)-2-((1S,3R)-1-(4-bromo-2-chlorophenyl)-4-oxo-5-azaspiro[2.4]heptan-5-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide (410 mg, 505 μmol) in 1,4-dioxane (25 mL) was added (2-methylimidazo[1,2-a]pyrimidin-6-yl) boronic acid (98.5 mg, 557 μmol), sodium carbonate (168 mg, 1.59 mmol), XPhos Pd G3 (86.1 mg, 102 μmol) and water (2.5 mL). The mixture was degassed and purged with N₂ 3 times, then stirred at 90° C. for 1 h. The reaction mixture was cooled down to RT, then diluted with water (100 mL) and extracted with EtOAc (80 mL×2), the organic layer was washed with sat. aq. NaCl (80 mL), dried over anhydrous Na₂SO₄, filtered and concentrated under reduced pressure. The residue was purified by silica gel chromatography eluting with 4 to 6% MeOH in DCM to obtain the product (360 mg), which was combined with 355 mg of product prepared as described above and purified by chiral SFC purification (Chiralpak OD 50×250 mm, 5 μm), eluting with EtOH (with 0.1% NH₃H₂O) to afford the title compound (510 mg, 61%) as a white solid. ES-MS m/z 824 (M+H).

### Example 149

### (R)-3-(1-(Difluoromethyl)-1H-pyrazol-4-yl)-2-((1S,3R)-1-(2-fluoro-4-(2-methylimidazo[1,2-a]pyrimidin-6-yl)phenyl)-4-oxo-5-azaspiro[2.4]heptan-5-yl)-N-(2-methyl-1-(5-(6-oxo-1,6-dihydropyridazin-3-yl)-3-(trifluoromethyl)-1H-pyrazol-1-yl)propan-2-yl)propanamide

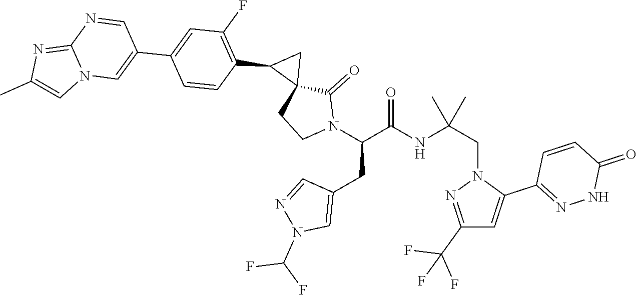 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4ccc(=O)[nH]n4)C5=O)c(F)c3)cnc2n1]

A mixture of (R)-2-((1S,3R)-1-(4-bromo-2-fluorophenyl)-4-oxo-5-azaspiro[2.4]heptan-5-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide (1.14 g, 43 mmol), (2-methylimidazo[1,2-a]pyrimidin-6-yl) boronic acid (530 mg, 2.99 mmol), Na₂CO₃ (342 μL, 4.76 mmol) and XPhos Pd G3 (251 mg, 297 μmol) in 1,4-dioxane (60 mL) and H₂O (7 mL) was degassed and refilled with N₂ 3 times. The mixture was heated to 90° C. for 2 h, then was quenched by the addition of H₂O (100 mL) and extracted with EtOAc (150 mL×2). The combined organic layers were washed with sat. aq. NaCl (100 mL), dried over Na₂SO₄, filtered, and concentrated under reduced pressure. The crude material was purified on silica gel eluting with 5-7% MeOH in DCM to give the title compound (680.7 mg, 56%) as a white solid. ES-MS m/z 808 (M+H).

### Example 150

### (3-(1-(2-((R)-2-((1S,3R)-1-(2-Chloro-4-(2-methylimidazo[1,2-a]pyrimidin-6-yl)phenyl)-4-oxo-5-azaspiro[2.4]heptan-5-yl)-3-(1-(difluoromethyl)-1H-pyrazol-4-yl)propanamido)-2-methylpropyl)-3-(trifluoromethyl)-1H-pyrazol-5-yl)-6-oxopyridazin-1 (6H)-yl)methyl dihydrogen phosphate

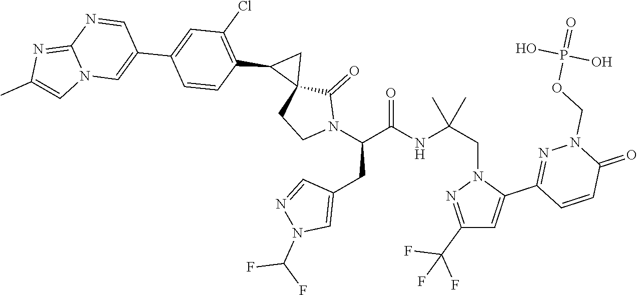 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4ccc(=O)n(COP(=O)(O)O)n4)C5=O)c(Cl)c3)cnc2n1]

To (R)-2-((1S,3R)-1-(2-chloro-4-(2-methylimidazo[1,2-a]pyrimidin-6-yl)phenyl)-4-oxo-5-azaspiro[2.4]heptan-5-yl)-3-(1-(difluoromethyl)-1H-pyrazol-4-yl)-N-(2-methyl-1-(5-(6-oxo-1,6-dihydropyridazin-3-yl)-3-(trifluoromethyl)-1H-pyrazol-1-yl)propan-2-yl)propanamide (180 mg, 218 μmol) was added sodium iodide (66 mg, 0.44 mmol), THF (3 mL), lithium tert-butoxide (31.5 mg, 394 μmol) and di-tert-butyl (chloromethyl)phosphate (113 mg, 102 μL, 438 μmol) at 0° C. The reaction was allowed to warm to RT overnight, then was quenched with water. The aqueous phase was extracted with EtOAc (×3) and the combined organic phases were dried over Na₂SO₄, then concentrated under reduced pressure. The residue was dissolved in DCM (3 mL) and then TFA (488 mg, 330 μL, 4.28 mmol) was added and the mixture was allowed to stir overnight. The reaction was then concentrated under reduced pressure, and purified by prep-HPLC (XSelect CSH C18, 50 mm×250 mm, 5 μm; mobile phase-gradient of 25-100% MeOH/ACN (1/1) in 0.1% aq. FA) to obtain the title compound (92 mg, 45%) as a white solid. ES-MS m/z 934 (M+H).

### Example 151

### (3-(1-(2-((R)-3-(1-(Difluoromethyl)-1H-pyrazol-4-yl)-2-((1S,3R)-1-(2-fluoro-4-(2-methylimidazo[1,2-a]pyrimidin-6-yl)phenyl)-4-oxo-5-azaspiro[2.4]heptan-5-yl)propanamido)-2-methylpropyl)-3-(trifluoromethyl)-1H-pyrazol-5-yl)-6-oxopyridazin-1 (6H)-yl)methyl dihydrogen phosphate

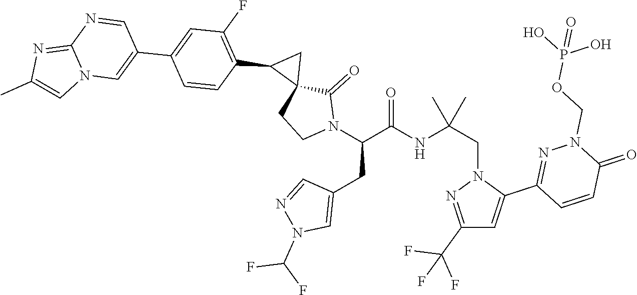 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4ccc(=O)n(COP(=O)(O)O)n4)C5=O)c(F)c3)cnc2n1]

The title compound was prepared as described in Example 150 from (R)-3-(1-(difluoromethyl)-1H-pyrazol-4-yl)-2-((1S,3R)-1-(2-fluoro-4-(2-methylimidazo[1,2-a]pyrimidin-6-yl)phenyl)-4-oxo-5-azaspiro[2.4]heptan-5-yl)-N-(2-methyl-1-(5-(6-oxo-1,6-dihydropyridazin-3-yl)-3-(trifluoromethyl)-1H-pyrazol-1-yl)propan-2-yl)propanamide. ES-MS m/z 918 (M+H).

### Example 152

### (R)-2-((1S,3R)-1-(2-Chloro-4-(2-methylimidazo[1,2-a]pyrimidin-6-yl)phenyl)-4-oxo-5-azaspiro[2.4]heptan-5-yl)-N-(2-methyl-1-(5-(2-methylpyrimidin-5-yl)-3-(trifluoromethyl)-1H-pyrazol-1-yl)propan-2-yl)-3-(1-(methyl-d3)-1H-pyrazol-4-yl)propanamide

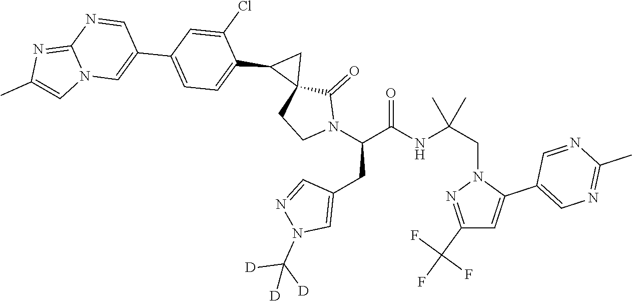

To (R)-2-((1S,3R)-1-(4-bromo-2-chlorophenyl)-4-oxo-5-azaspiro[2.4]heptan-5-yl)-N-(2-methyl-1-(5-(2-methylpyrimidin-5-yl)-3-(trifluoromethyl)-1H-pyrazol-1-yl)propan-2-yl)-3-(1-(methyl-d3)-1H-pyrazol-4-yl)propanamide (500 mg, 678 μmol) was added (2-methylimidazo[1,2-a]pyrimidin-6-yl) boronic acid (150 mg, 848 μmol), bis(di-tert-butyl (4-dimethylaminophenyl)phosphine)dichloropalladium (II) (43 mg, 61 μmol), and Na₂CO₃ (244 mg, 2.30 mmol). MeOH (7 mL) was added, and the reaction mixture was sparged with argon while stirring at RT. After 45 min, sparging was discontinued and the reaction mixture was stirred at 70° C. under argon for 105 min, cooled to RT, and treated with SiliaMetS® thiol resin (Silicycle, 1.46 mmol/g, 500 mg). The resulting suspension was stirred at RT overnight, filtered through diatomaceous earth, and the filter cake was rinsed with ACN (30 mL). The filtrate was concentrated under reduced pressure. The residue was purified by reverse-phase flash chromatography using a gradient of 35-70% ACN in 10 mM aq. NH₄HCO₃₊₅% MeOH to give the title compound (335.9 mg, 61%) as a white fluffy solid. ES-MS m/z 789 (M+H).

### Example 153

### (R)-3-(1-(Difluoromethyl)-1H-pyrazol-4-yl)-N-(2-methyl-1-(5-(1-methyl-6-oxo-1,6-dihydropyridin-3-yl)-3-(trifluoromethyl)-1H-pyrazol-1-yl)propan-2-yl)-2-((trans)-1-(4-(2-methylimidazo[1,2-a]pyrimidin-6-yl)-2-(trifluoromethyl)phenyl)-4-oxo-5-azaspiro[2.4]heptan-5-yl)propanamide (Isomer 1)

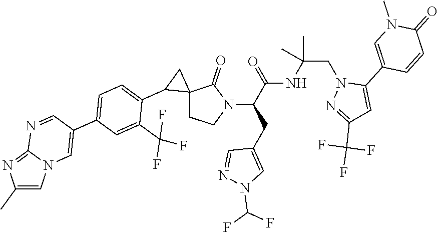 [SMILES: Cc1cn2cc(-c3ccc(C4CC45CCN([C@H](Cc4cnn(C(F)F)c4)C(=O)NC(C)(C)Cn4nc(C(F)(F)F)cc4-c4ccc(=O)n(C)c4)C5=O)c(C(F)(F)F)c3)cnc2n1]

To a solution of (4-((trans)-5-((R)-3-(1-(difluoromethyl)-1H-pyrazol-4-yl)-1-((2-methyl-1-(5-(1-methyl-6-oxo-1,6-dihydropyridin-3-yl)-3-(trifluoromethyl)-1H-pyrazol-1-yl)propan-2-yl)amino)-1-oxopropan-2-yl)-4-oxo-5-azaspiro[2.4]heptan-1-yl)-3-(trifluoromethyl)phenyl) boronic acid (mixture of isomers, 349.5 mg, 446 μmol) in 1,4-dioxane (3.5 mL) was added 6-bromo-2-methylimidazo[1,2-a]pyrimidine (227.2 mg, 1.07 mmol), Pd(dppf)Cl₂ (DCM adduct, 36.8 mg, 45.1 μmol), Cs₂CO₃ (453.4 mg, 1.40 mmol) and H₂O (1.0 mL). The reaction mixture was heated to 90° C. under N₂ for 2 h, then was cooled to RT. The aqueous layer was separated from the cooled mixture and washed with EtOAc. To the organic mixture was then added 4 g of Silicycle SiliMetS® Thiol metal scavenger. After 15 min the mixture was filtered, and the residue was washed with EtOAc and DCM, then concentrated under reduced pressure. The crude product was purified by prep-HPLC (UPLC CSH XSelect C18, 50 mm×250 mm, 5 μm) using a gradient of 5-95% ACN in 10 mM aq. NH₄HCO₃ followed by chiral SFC purification (Chiralpak IK 30×250 mm, 5 μm), eluting with MeOH to afford the title compound (18.8 mg, 4.8%) as the first eluting isomer. ES-MS m/z 871 (M+H).

### Example 154

### (R)—N-(1-(5-(2H-1,2,3-Triazol-4-yl)-3-(trifluoromethyl)-1H-pyrazol-1-yl)-2-methylpropan-2-yl)-2-((1S,3R)-1-(2-chloro-4-(1-methyl-2-oxo-1,2-dihydropyrimidin-5-yl)phenyl)-4-oxo-5-azaspiro[2.4]heptan-5-yl)-3-(1-(difluoromethyl)-1H-pyrazol-4-yl)propanamide

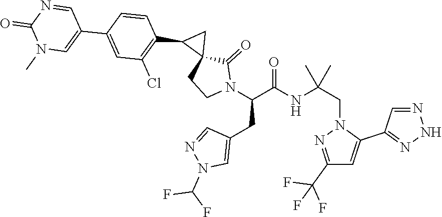 [SMILES: Cn1cc(-c2ccc([C@H]3C[C@@]34CCN([C@H](Cc3cnn(C(F)F)c3)C(=O)NC(C)(C)Cn3nc(C(F)(F)F)cc3-c3cn[nH]n3)C4=O)c(Cl)c2)cnc1=O]

To a mixture of (2R)-2-((1S,3R)-1-(2-chloro-4-(1-methyl-2-oxo-1,2-dihydropyrimidin-5-yl)phenyl)-4-oxo-5-azaspiro[2.4]heptan-5-yl)-3-(1-(difluoromethyl)-1H-pyrazol-4-yl)-N-(2-methyl-1-(5-(2-(tetrahydro-2H-pyran-2-yl)-2H-1,2,3-triazol-4-yl)-3-(trifluoromethyl)-1H-pyrazol-1-yl)propan-2-yl)propanamide (99 mg, 54% purity, 62 μmol) in 1,4-dioxane (2 mL) was added 2.0 M HCl in 1,4-dioxane (1.00 mL, 2.00 mmol). The mixture was stirred at 20° C. for 2 h, filtered, and the filter cake was washed with DCM (8 mL×3). The filter cake was dried under reduced pressure and purified by preparative HPLC (YMC Triart C18, 250×30 mm, 5 μm) using a gradient of 30-60% ACN/water (+0.225% FA) to afford the title compound (22.79 mg, 47%) as a white solid. ES-MS m/z 774 (M+H).

### Example 155

### (1S,2S)-2-(3-Chloro-6-cyano-1-methyl-1H-pyrazolo[4,3-b]pyridin-5-yl)-N—((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl)cyclopropane-1-carboxamide

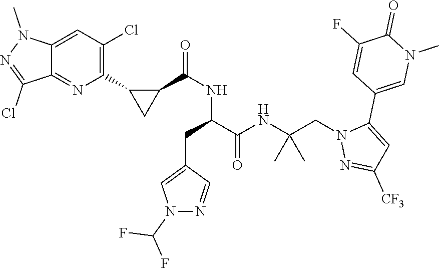 [SMILES: Cn1cc(-c2cc(C(F)(F)F)nn2CC(C)(C)NC(=O)[C@@H](Cc2cnn(C(F)F)c2)NC(=O)[C@H]2C[C@@H]2c2nc3c(Cl)nn(C)c3cc2Cl)cc(F)c1=O]

To (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (281.0 mg, 541.0 μmol) was added (1S,2S)-2-(3-chloro-6-cyano-1-methyl-1H-pyrazolo[4,3-b]pyridin-5-yl)cyclopropane-1-carboxylic acid (772.7 mg, 34% Wt, 949.5 μmol), HATU (376.4 mg, 989.9 μmol), DIEA (223 mg, 300 μL, 1.72 mmol) and DMF (4 mL). The suspension was stirred at 0° C. for 5 min and then stirred at RT for 2 h. The mixture was dissolved in DMF and then filtered through a 0.45 μm syringe filter and purified by prep-HPLC (Charged Surface Hybrid XSelect, C18, 50 mm×250 mm, 5 μm) using a gradient of 40-95% ACN/water (with 0.1% FA) to afford the title compound (63.3 mg, 15%) as a white powder. ES-MS m/z 778 (M+H).

The compounds in the following table were prepared as described in Example 155 using the appropriate carboxylic acid. Reactants can be added in different orders or in differing equivalency and differing methods can be used to work up or purify the compounds (normal phase, or high, neutral or low pH reversed phase). Such variances would be apparent to one skilled in the art.

TABLE 42

| Ex- | | | ES-MS |
|---|---|---|---|
| ample | Chemical Name | Structure | m/z |
| 156 | (1S,2S)-N-((R)-3-(1- (Difluoromethyl)-1H- pyrazol-4-yl)-1-((1-(5-(5- fluoro-1-methyl-6-oxo-1,6- dihydropyridin-3-yl)-3- (trifluoromethyl)-1H- pyrazol-1-yl)-2- methylpropan-2-yl)amino)- 1-oxopropan-2-yl)-2-(6- fluoro-1-methyl-1H- pyrazolo[4,3-b]pyridin-5- yl)cyclopropane-1- carboxamide | 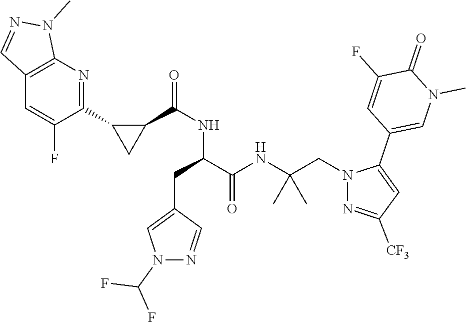 [SMILES: Cn1cc(-c2cc(C(F)(F)F)nn2CC(C)(C)NC(=O)[C@@H](Cc2cnn(C(F)F)c2)NC(=O)[C@H]2C[C@@H]2c2nc3c(cnn3C)cc2F)cc(F)c1=O] | 737 (M + H) |
| 157 | (1S,2S)-N-((R)-3-(1- (Difluoromethyl)-1H- pyrazol-4-yl)-1-((1-(5-(5- fluoro-1-methyl-6-oxo-1,6- dihydropyridin-3-yl)-3- (trifluoromethyl)-1H- pyrazol-1-yl)-2- methylpropan-2-yl)amino)- 1-oxopropan-2-yl)-2-(6- fluoro-1-methyl-1H- pyrazolo[4,3-b]pyridin-5- yl)cyclopropane-1- carboxamide | 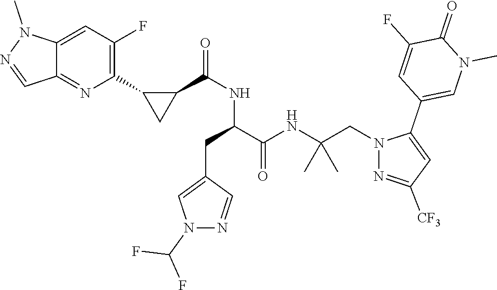 [SMILES: Cn1cc(-c2cc(C(F)(F)F)nn2CC(C)(C)NC(=O)[C@@H](Cc2cnn(C(F)F)c2)NC(=O)[C@H]2C[C@@H]2c2nc3cnn(C)c3cc2F)cc(F)c1=O] | 737 (M + H) |

### Example 158

### (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

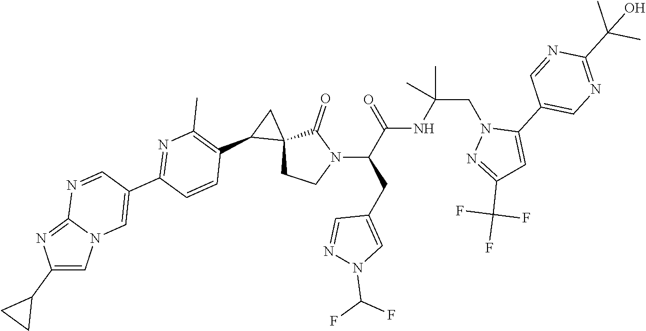 [SMILES: Cc1nc(-c2cnc3nc(C4CC4)cn3c2)ccc1[C@H]1C[C@@]12CCN([C@H](Cc1cnn(C(F)F)c1)C(=O)NC(C)(C)Cn1nc(C(F)(F)F)cc1-c1cnc(C(C)(C)O)nc1)C2=O]

To (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)propanoic acid (490 mg, 850 μmol) was added 2-(5-(1-(2-amino-2-methylpropyl)-3-(trifluoromethyl)-1H-pyrazol-5-yl)pyrimidin-2-yl)propan-2-ol hydrochloride (409 mg, 1.08 mmol), and DMA (5.0 mL). The mixture was stirred at RT for 10 min, then N-methylmorpholine (500 μL, 4.55 mmol) was added, followed by DEPBT (509 mg, 1.70 mmol). The reaction was stirred at RT for 2 days, diluted with H₂O (3 mL) and a minimal amount of MeOH. The solution was purified by reverse-phase flash chromatography (C18, gradient 0-100% ACN in 10 mM aq. NH₄HCO₃+5% MeOH) to provide the title compound (622 mg, 80%) as an off-white solid. ES-MS m/z 873.4 (M+H).

### Example 159

### (R)-2-((1R,3R)-1-(6-(2-Cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)-N-(1-(5-(6-(2-hydroxypropan-2-yl)pyridin-3-yl)-3-(trifluoromethyl)-1H-pyrazol-1-yl)-2-methylpropan-2-yl)propanamide

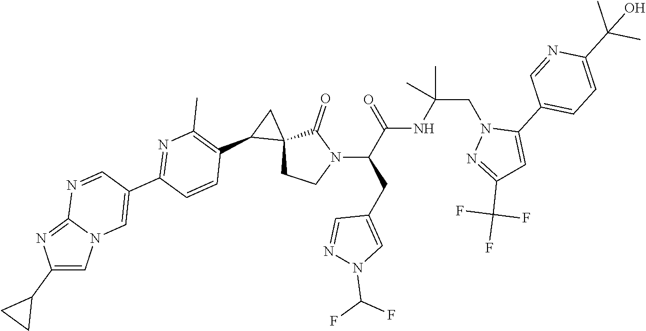 [SMILES: Cc1nc(-c2cnc3nc(C4CC4)cn3c2)ccc1[C@H]1C[C@@]12CCN([C@H](Cc1cnn(C(F)F)c1)C(=O)NC(C)(C)Cn1nc(C(F)(F)F)cc1-c1ccc(C(C)(C)O)nc1)C2=O]

To (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)propanoic acid (589 mg, 1.02 mmol, 95% purity) was added 2-(5-(1-(2-amino-2-methylpropyl)-3-(trifluoromethyl)-1H-pyrazol-5-yl)pyridin-2-yl)propan-2-ol hydrochloride (494 mg, 1.30 mmol), DMA (6.0 mL), and N-methylmorpholine (700 μL, 6.37 mmol). The resulting mixture was stirred at RT for 5 min, then DEPBT (653 mg, 2.18 mmol) was added. The reaction was stirred at RT for 3 days, diluted with water (3 mL) and loaded onto a celite cartridge. Reverse-phase flash chromatography (C18, gradient 0-75% ACN in 10 mM aq. NH₄HCO₃₊₅% MeOH) provided the title compound (392.8 mg, 42%) as a white solid. ES-MS m/z 872.4 (M+H).

### Example 160

### (R)-3-(1-(difluoromethyl)-1H-pyrazol-4-yl)-N-(1-(5-(2-(2-hydroxypropan-2-yl)pyridin-4-yl)-3-(trifluoromethyl)-1H-pyrazol-1-yl)-2-methylpropan-2-yl)-2-((1R,3R)-1-(6-(2-(methoxymethyl) imidazo[1,2-a]pyrimidin-6-yl)-2-methylpyridin-3-yl)-4-oxo-5-azaspiro[2.4]heptan-5-yl)propanamide

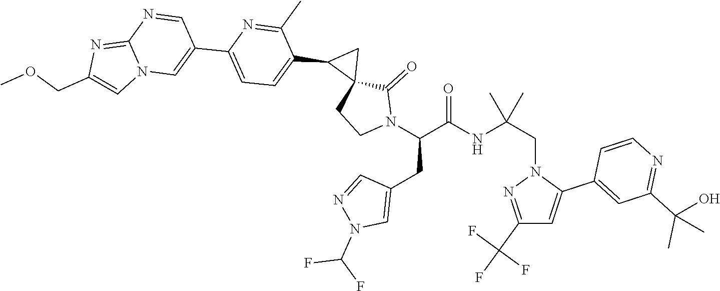 [SMILES: COCc1cn2cc(-c3ccc([C@H]4C[C@@]45CCN([C@H](Cc4cnn(C(F)F)c4)C(=O)NC(C)(C)Cn4nc(C(F)(F)F)cc4-c4ccnc(C(C)(C)O)c4)C5=O)c(C)n3)cnc2n1]

The title compound was prepared as described in Example 159 from (R)-3-(1-(difluoromethyl)-1H-pyrazol-4-yl)-2-((1R,3R)-1-(6-(2-(methoxymethyl) imidazo[1,2-a]pyrimidin-6-yl)-2-methylpyridin-3-yl)-4-oxo-5-azaspiro[2.4]heptan-5-yl)propanoic acid (97.3 mg, 176 μmol) and 2-(4-(1-(2-amino-2-methylpropyl)-3-(trifluoromethyl)-1H-pyrazol-5-yl)pyridin-2-yl)propan-2-ol dihydrochloride (139 mg, 218 μmol, 65% purity). ES-MS m/z 876.4 (M+H).

### Example 161

### (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-N-(1-(5-(1-cyclopropyl-2-oxo-1,2-dihydropyrimidin-4-yl)-3-(trifluoromethyl)-1H-pyrazol-1-yl)-2-methylpropan-2-yl)-3-(1-(difluoromethyl)-1H-pyrazol-4-yl)propanamide

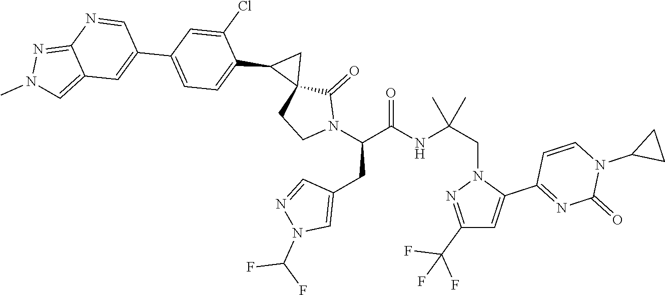 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccn(C6CC6)c(=O)n4)C5=O)c(Cl)c3)cnc2n1]

To a solution of (R)-2-((1R,3S)-1-(2-chloro-4-(2-methyl-2H-pyrazolo[3,4-b]pyridin-5-yl)phenyl)-4-oxo-5-azaspiro[2.4]heptan-5-yl)-3-(1-(difluoromethyl)-1H-pyrazol-4-yl)propanoic acid (70 mg, 0.12 mmol) in DMA (2 mL) was added HATU (58 mg, 0.15 mmol), DIPEA (0.14 mL, 0.81 mmol) and 4-(1-(2-amino-2-methylpropyl)-3-(trifluoromethyl)-1H-pyrazol-5-yl)-1-cyclopropylpyrimidin-2(1H)-one hydrochloride (48 mg, 0.13 mmol) at 20° C. The resulting solution was stirred at 20° C. for 1 h. The reaction mixture was diluted with H₂O (10 mL) and extracted with EtOAc (10 mL×2). The combined organic layers were washed with sat. aq. NaCl (20 mL×3), dried over Na₂SO₄, and filtered. The filtrate was concentrated under reduced pressure to give the residue as a yellow solid. The residue was purified by preparative HPLC, eluting with 36-66% ACN in H₂O (with 0.225% FA) to give the title compound as a white solid (80 mg, 74%). ES-MS m/z 864.3 (M+H)

The compounds in the following table were prepared as described in Example 161 using the appropriate amine and carboxylic acid. Reactants can be added in different orders or in differing equivalency and differing methods can be used to work up or purify the compounds (normal phase, or high, neutral or low pH reversed phase). Such variances would be apparent to one skilled in the art.

TABLE 49

| Ex- | | | ES-MS |
|---|---|---|---|
| ample | Chemical Name | Structure | m/z |
| 162 | (R)-2-((1S,3R)-1-(2- chloro-4-(2-methyl- 2H-pyrazolo[3,4- b]pyridin-5- yl)phenyl)-4-oxo-5- azaspiro[2.4] heptan-5-yl)- 3-(1-(difluoro- methyl)-1H- pyrazol-4-yl)-N- (1-(5-(4-(dimethyl- phosphoryl)phenyl)- 3-(trifluoro- methyl)-1H- pyrazol-1-yl)-2- methylpropan-2- yl)propanamide | 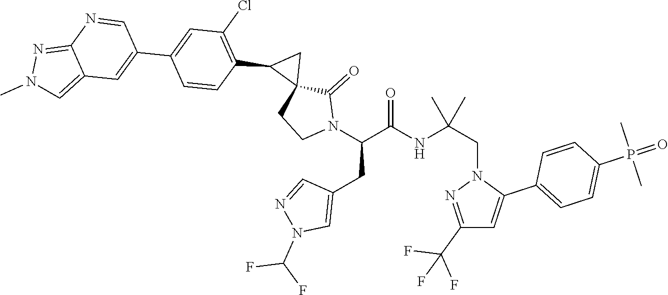 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4-c4ccc(P(C)(C)=O)cc4)C5=O)c(Cl)c3)cnc2n1] | 882.3 (M + H) |
| 163^{a} | (R)-2-((1S,3R)-1-(2- chloro-4-(2-methyl- 2H-pyrazolo[3,4- b]pyridin-5- yl)phenyl)-4-oxo-5- azaspiro[2.4] heptan-5-yl)- 3-(1-(difluoro- methyl)-1H- pyrazol-4-yl)- N-(1-(5-(dimethyl- phosphoryl)-3- (trifluoro- methyl)-1H- pyrazol-1-yl)-2- methylpropan-2- yl)propanamide | 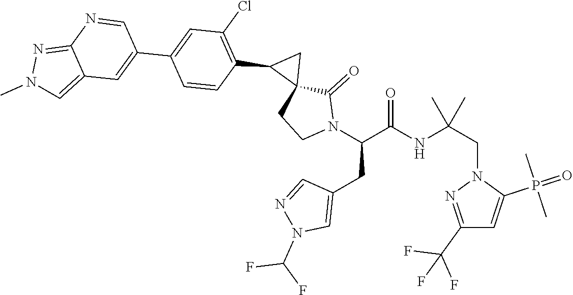 [SMILES: Cn1cc2cc(-c3ccc([C@H]4C[C@@]45CCN([C@H](Cc4cnn(C(F)F)c4)C(=O)NC(C)(C)Cn4nc(C(F)(F)F)cc4P(C)(C)=O)C5=O)c(Cl)c3)cnc2n1] | 806.2 (M + H) |

^{a}Reaction was carried out at 0 C. for 0.5 h.

### Example 164

### (R)-3-(1-(difluoromethyl)-1H-pyrazol-4-yl)-2-((1R,3R)-1-(6-(2-ethyl-2H-pyrazolo[3,4-b]pyridin-5-yl)-2-methylpyridin-3-yl)-4-oxo-5-azaspiro[2.4]heptan-5-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide

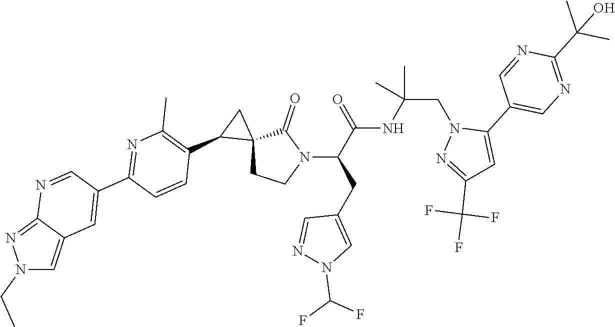 [SMILES: CCn1cc2cc(-c3ccc([C@H]4C[C@@]45CCN([C@H](Cc4cnn(C(F)F)c4)C(=O)NC(C)(C)Cn4nc(C(F)(F)F)cc4-c4cnc(C(C)(C)O)nc4)C5=O)c(C)n3)cnc2n1]

To (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide (119.5 mg, 159.3 μmol) in 1,4-dioxane (2 mL) was added 2-ethyl-5-(4,4,5,5-tetramethyl-1,3,2-dioxaborolan-2-yl)-2H-pyrazolo[3,4-b]pyridine (52 mg, 0.19 mmol) in 1,4-dioxane (2 mL), Cs₂CO₃ (169 mg, 517 μmol), PdCl₂dppf CH₂Cl₂ (17.5 mg, 21.4 μmol) and water (1 mL). The mixture was purged with N₂ for 3 min and heated to 90° C. for 1.5 h. The reaction mixture was cooled to RT and 150 mg SiliaMetS® metal scavenger resin was added, and the suspension was filtered, rinsing with EtOAc. The combined filtrate was concentrated under reduced pressure, re-dissolved in DMF, filtered by syringe filter, and purified by UPLC CSH (C18, 2.1 mm×50 mm, 1.7 μm, 0.8 mL/min) using a gradient of 5-95% ACN in water (10 mM NH₄OAc) to obtain the title compound (70.7 mg, 51%) as an off-white powder. ES-MS m/z 861.6 (M+H).

### Example 165

(R)-2-((1S,3R)-1-(2-chloro-4-(2-methylimidazo[1,2-a]pyrimidin-6-yl)phenyl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide

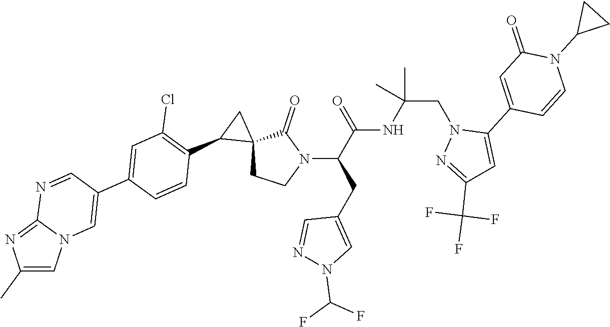 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4ccn(C6CC6)c(=O)c4)C5=O)c(Cl)c3)cnc2n1]

To a mixture of (2-methylimidazo[1,2-a]pyrimidin-6-yl) boronic acid (71.2 mg, 402 μmol), Cs₂CO₃ (130 mg, 400 μmol), PdCl₂dppf CH₂Cl₂ (22.2 mg, 27.2 μmol)), (R)-2-((1R,3S)-1-(4-bromo-2-chlorophenyl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide (105 mg, 127 μmol) in 1,4-dioxane (3 mL) was added water (1 mL). The mixture was purged with N₂ for 3 min and heated at 90° C. for 2 h. The reaction mixture was cooled to RT and 250 mg SiliaMetS® metal scavenger resin was added. The suspension was stirred for 0.5 h, filtered, washing with EtOAc and MeOH. The combined filtrate was concentrated under reduced pressure, re-dissolved in DMF, filtered by syringe filter, and purified by XSelect CSH (C18, 50 mm×250 mm, 5 μm, 100 mL/min) using a gradient of 5-95% ACN in water (10 mM NH₄OAc) to obtain the title compound (79.5 mg, 70%) as an off-white powder. ES-MS m/z 863.4, 865.4 (M+H).

### Example 166

### (R)—N-(1-(5-(1-cyclopropyl-2-oxo-1,2-dihydropyridin-4-yl)-3-(trifluoromethyl)-1H-pyrazol-1-yl)-2-methylpropan-2-yl)-2-((trans)-1-(2-cyclopropyl-6-(2-cyclopropylimidazo[1,2-a]pyrimidin-6-yl)pyridin-3-yl)-4-oxo-5-azaspiro[2.4]heptan-5-yl)-3-(1-(difluoromethyl)-1H-pyrazol-4-yl)propanamide

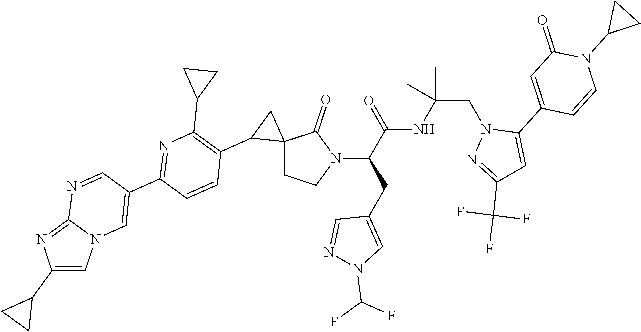 [SMILES: CC(C)(Cn1nc(C(F)(F)F)cc1-c1ccn(C2CC2)c(=O)c1)NC(=O)[C@@H](Cc1cnn(C(F)F)c1)N1CCC2(CC2c2ccc(-c3cnc4nc(C5CC5)cn4c3)nc2C2CC2)C1=O]

(2-cyclopropylimidazo[1,2-a]pyrimidin-6-yl) boronic acid (96.3 mg, 475 μmol) was dissolved in 1,4-dioxane (3 mL) and Cs₂CO₃ (131 mg, 404 μmol), Pd(dppf)Cl₂·CH₂Cl₂ (17.0 mg, 20.8 μmol), (R)-2-((trans)-1-(6-bromo-2-cyclopropylpyridin-3-yl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide (100 mg, 122 umol, mixture of isomers and 2-bromo and 2-chloropyridyl analogues) and water (1 mL) were added and the mixture was stirred under N₂ for 3 mins. The reaction was heated to 90° C. for 1 h, then was cooled and diluted with DCM and water. The aqueous layer was extracted with DCM (3×), then the organic layers were combined and dried over sodium sulfate and concentrated. The crude product was purified on silica, eluting with 0-100% 3:1 EtOAc:EtOH in heptane, followed by preparative HPLC (XSelect CSH C18, 250×50 mm, 5 μm, 100 mL/min) using a gradient of 5-95% ACN/water (w/0.1% FA) and chiral SFC purification (Chiralpak IE 30×250 mm×5 μM, 52 mL/min), eluting with MeOH (with 10 mM NH₄OAc) to afford the title compound (15.6 mg, 13%) as the second eluting isomer. ES-MS m/z 896.4 (M+H).

### Example 167

(R)—N-(1-(5-(1-cyclopropyl-2-oxo-1,2-dihydropyridin-4-yl)-3-(trifluoromethyl)-1H-pyrazol-1-yl)-2-methylpropan-2-yl)-2-((trans)-1-(2-cyclopropyl-6-(2-methylimidazo[1,2-a]pyrimidin-6-yl)pyridin-3-yl)-4-oxo-5-azaspiro[2.4]heptan-5-yl)-3-(1-(difluoromethyl)-1H-pyrazol-4-yl)propanamide

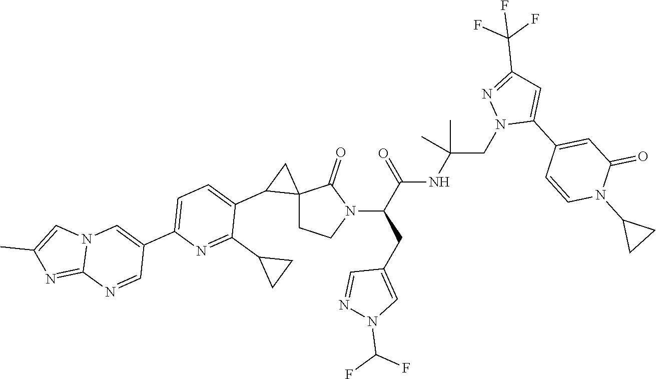 [SMILES: Cc1cn2cc(-c3ccc(C4CC45CCN([C@H](Cc4cnn(C(F)F)c4)C(=O)NC(C)(C)Cn4nc(C(F)(F)F)cc4-c4ccn(C6CC6)c(=O)c4)C5=O)c(C4CC4)n3)cnc2n1]

The title compound was prepared in a similar manner as described in Example 166 using (R)-2-((trans)-1-(6-bromo-2-cyclopropylpyridin-3-yl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide (60 mg, 73 μmol, mixture of isomers and mixture of 2-bromo and 2-chloropyridyl analogues) and (2-methylimidazo[1,2-a]pyrimidin-6-yl) boronic acid (39 mg, 0.22 mmol). The title compound was obtained as the second eluting isomer. ES-MS m/z 870 (M+H).

### Example 168

### (R)-3-(1-(difluoromethyl)-1H-pyrazol-4-yl)-N-(1-(5-(6-(2-hydroxypropan-2-yl)pyridin-3-yl)-3-(trifluoromethyl)-1H-pyrazol-1-yl)-2-methylpropan-2-yl)-2-((1R,3R)-1-(6-(2-(methoxymethyl) imidazo[1,2-a]pyrimidin-6-yl)-2-methylpyridin-3-yl)-4-oxo-5-azaspiro[2.4]heptan-5-yl)propanamide

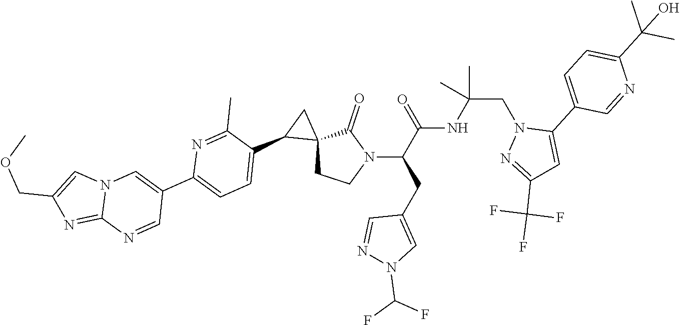 [SMILES: COCc1cn2cc(-c3ccc([C@H]4C[C@@]45CCN([C@H](Cc4cnn(C(F)F)c4)C(=O)NC(C)(C)Cn4nc(C(F)(F)F)cc4-c4ccc(C(C)(C)O)nc4)C5=O)c(C)n3)cnc2n1]

A mixture of (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)-N-(1-(5-(6-(2-hydroxypropan-2-yl)pyridin-3-yl)-3-(trifluoromethyl)-1H-pyrazol-1-yl)-2-methylpropan-2-yl)propanamide (403 mg, 538 μmol), (2-(methoxymethyl) imidazo[1,2-a]pyrimidin-6-yl) boronic acid (286 mg, 1.38 mmol), PdCl₂dppf CH₂Cl₂ (45 mg, 55 μmol), and Cs₂CO₃ (460 mg, 1.41 mmol) in 1,4-dioxane (8 mL) and H₂O (2 mL) was purged with N₂ for 10 min, then stirred at 85° C. overnight. The mixture was diluted with EtOAc, filtered through celite, and concentrated under reduced pressure. Reverse-phase flash chromatography (C18, gradient 20-45% ACN in 0.1% aq. FA) provided the title compound (185.6 mg, 36%) as a pale brown solid. ES-MS m/z 876.4 (M+H).

### Example 169

### (R)—N-(1-(5-(1-cyclopropyl-2-oxo-1,2-dihydropyridin-4-yl)-3-(trifluoromethyl)-1H-pyrazol-1-yl)-2-methylpropan-2-yl)-2-((1R,3R)-1-(6-(2-cyclopropyl-2H-pyrazolo[3,4-b]pyridin-5-yl)-2-methylpyridin-3-yl)-4-oxo-5-azaspiro[2.4]heptan-5-yl)-3-(1-(difluoromethyl)-1H-pyrazol-4-yl)propanamide

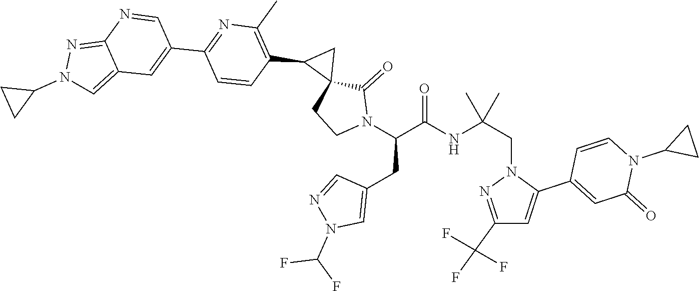 [SMILES: Cc1nc(-c2cnc3nn(C4CC4)cc3c2)ccc1[C@H]1C[C@@]12CCN([C@H](Cc1cnn(C(F)F)c1)C(=O)NC(C)(C)Cn1nc(C(F)(F)F)cc1-c1ccn(C3CC3)c(=O)c1)C2=O]

A solution of crude 2-cyclopropyl-5-(4,4,5,5-tetramethyl-1,3,2-dioxaborolan-2-yl)-2H-pyrazolo[3,4-b]pyridine (61.6 mg, 216 μmol) in 1,4-dioxane (2 mL) was charged with a solution of (R)-2-((1R,3R)-1-(6-chloro-2-methylpyridin-3-yl)-4-oxo-5-azaspiro[2.4]heptan-5-yl)-N-(1-(5-(1-cyclopropyl-2-oxo-1,2-dihydropyridin-4-yl)-3-(trifluoromethyl)-1H-pyrazol-1-yl)-2-methylpropan-2-yl)-3-(1-(difluoromethyl)-1H-pyrazol-4-yl)propanamide (150 mg, 143 μmol, 71% purity) in 1,4-dioxane (1 mL), PdCl₂dppf CH₂Cl₂ (17 mg, 21 μmol), and a solution of Cs₂CO₃ (150 mg, 460 μmol) in water (300 μL). The reaction mixture was stirred at 80° C. under Ar overnight, cooled to RT, treated with SiliaMetS thiol resin (Silicycle, 1.46 mmol/g, 300 mg), stirred at RT for 2 h, filtered, and concentrated at 40° C. under N₂. The filtrate was re-suspended in ACN (2 mL), filtered, and purified by reverse-phase HPLC (XSelect CSH C18, 50×250 mm, 5 μm, 5-95% ACN in 0.1% aq. FA) to provide the title compound (88.7 mg, 70%) as a white solid. ES-MS m/z 870.6 (M+H).

### Example 170

### (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide

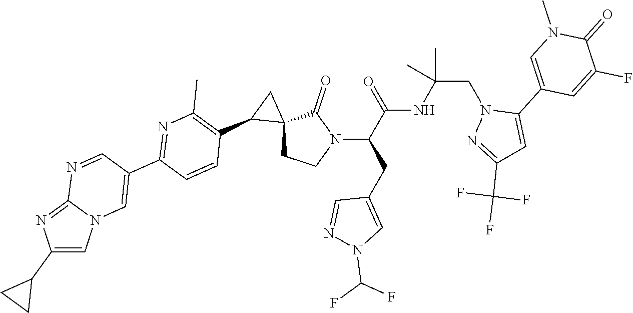 [SMILES: Cc1nc(-c2cnc3nc(C4CC4)cn3c2)ccc1[C@H]1C[C@@]12CCN([C@H](Cc1cnn(C(F)F)c1)C(=O)NC(C)(C)Cn1nc(C(F)(F)F)cc1-c1cc(F)c(=O)n(C)c1)C2=O]

To (2-cyclopropylimidazo[1,2-a]pyrimidin-6-yl) boronic acid (408 mg, 2.01 mmol) in 1,4-dioxane (6 mL) was added (R)-2-((1R,3R)-1-(6-chloro-2-methylpyridin-3-yl)-4-oxo-5-azaspiro[2.4]heptan-5-yl)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (1.87 g, 74% purity, 1.87 mmol), 1,4-dioxane (6 mL), Cs₂CO₃ (1.87 g, 5.75 mmol), PdCl₂dppf CH₂Cl₂ (229 mg, 281 μmol) and H₂O (3 mL). The mixture was purged with N₂ for 3 min, heated to 90° C. for 2.5 h, then cooled to RT and partitioned between water (50 mL) and EtOAc (50 mL). The layers were separated, and the aqueous layer was extracted with EtOAc (50 mL×2). The combined organic layers were washed with sat. aq. NaCl (50 mL), dried over Na₂SO₄, and concentrated. The crude product was purified on silica, eluting with 0-100% (3:1) EtOAc:EtOH in heptane, followed by preparative HPLC (XSelect CSH C18, 50 mm×250 mm, 5 μm, 100 mL/min) using a gradient of 5-95% ACN in water (0.1% aq. FA) to obtain the title compound (481 mg, 29%) as a white powder. ES-MS m/z 862.4 (M+H)

The compounds in the following table were prepared as described in Example 170 using the appropriate arylboronic acid and aryl halide. Reactants can be added in different orders or in differing equivalency and differing methods can be used to work up or purify the compounds (normal phase, or high, neutral or low pH reversed phase). Such variances would be apparent to one skilled in the art.

| Ex- | | | ES-MS |
|---|---|---|---|
| ample | Chemical Name | Structure | m/z |
| 171^{a} | (R)-N-(1-(5-(1- cyclopropyl-2-oxo-1,2- dihydropyridin-4-yl)-3- (trifluoromethyl)-1H- pyrazol-1-yl)-2- methylpropan-2-yl)-3-(1- (difluoromethyl)-1H- pyrazol-4-yl)-2-((1R,3R)- 1-(6′-(2-hydroxypropan-2- yl)-6-methyl-[2,3′- bipyridin]-5-yl)-4-oxo-5- azaspiro[2.4]heptan-5- yl)propanamide | 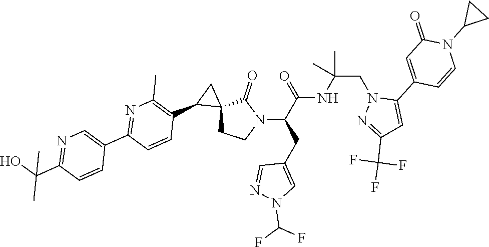 [SMILES: Cc1nc(-c2ccc(C(C)(C)O)nc2)ccc1[C@H]1C[C@@]12CCN([C@H](Cc1cnn(C(F)F)c1)C(=O)NC(C)(C)Cn1nc(C(F)(F)F)cc1-c1ccn(C3CC3)c(=O)c1)C2=O] | 848.4 (M + H) |
| 172^{b} | (R)-N-(1-(5-(1- cyclopropyl-2-oxo-1,2- dihydropyridin-4-yl)-3- (trifluoromethyl)-1H- pyrazol-1-yl)-2- methylpropan-2-yl)-3-(1- (difluoromethyl)-1H- pyrazol-4-yl)-2-((1R,3R)- 1-(6′-methoxy-6-methyl- [2,3′-bipyridin]-5-yl)-4- oxo-5-azaspiro[2.4]heptan- 5-yl)propanamide | 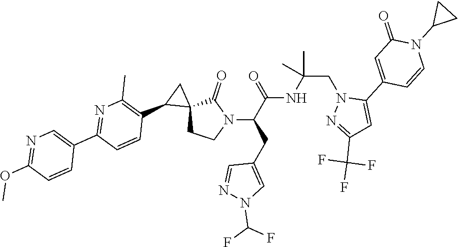 [SMILES: COc1ccc(-c2ccc([C@H]3C[C@@]34CCN([C@H](Cc3cnn(C(F)F)c3)C(=O)NC(C)(C)Cn3nc(C(F)(F)F)cc3-c3ccn(C5CC5)c(=O)c3)C4=O)c(C)n2)cn1] | 820.4 (M + H) |
| 173^{c} | (R)-N-(1-(5-(1- cyclopropyl-2-oxo-1,2- dihydropyridin-4-yl)-3- (trifluoromethyl)-1H- pyrazol-1-yl)-2- methylpropan-2-yl)-3-(1- (difluoromethyl)-1H- pyrazol-4-yl)-2-((1R,3R)- 1-(6-(2-methoxypyrimidin- 5-yl)-2-methylpyridin-3- yl)-4-oxo-5- azaspiro[2.4]heptan-5- yl)propanamide | 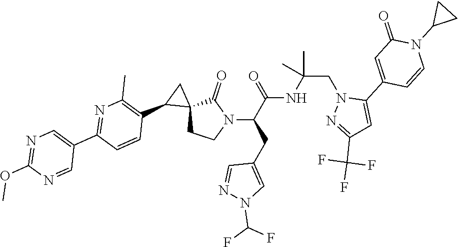 [SMILES: COc1ncc(-c2ccc([C@H]3C[C@@]34CCN([C@H](Cc3cnn(C(F)F)c3)C(=O)NC(C)(C)Cn3nc(C(F)(F)F)cc3-c3ccn(C5CC5)c(=O)c3)C4=O)c(C)n2)cn1] | 821.4 (M + H) |
| 174^{d} | (R)-N-(1-(5-(1- cyclopropyl-2-oxo-1,2- dihydropyridin-4-yl)-3- (trifluoromethyl)-1H- pyrazol-1-yl)-2- methylpropan-2-yl)-3-(1- (difluoromethyl)-1H- pyrazol-4-yl)-2-((1R,3R)- 1-(5′-fluoro-6′-methoxy-6- methyl-[2,3′-bipyridin]-5- yl)-4-oxo-5- azaspiro[2.4]heptan-5- yl)propanamide | 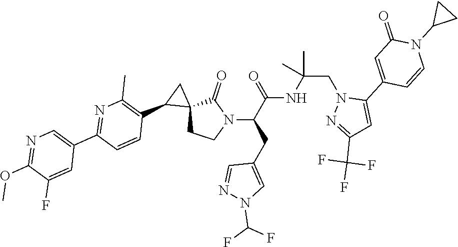 [SMILES: COc1ncc(-c2ccc([C@H]3C[C@@]34CCN([C@H](Cc3cnn(C(F)F)c3)C(=O)NC(C)(C)Cn3nc(C(F)(F)F)cc3-c3ccn(C5CC5)c(=O)c3)C4=O)c(C)n2)cc1F] | 838.6 (M + H) |
| 175^{e} | (R)-2-((1R,3R)-1-(6-(2- cyclopropylimidazo[1,2- a]pyrimidin-6-yl)-2- methylpyridin-3-yl)-4-oxo- 5-azaspiro[2.4]heptan-5- yl)-N-(2-methyl-1-(5-(6- methylpyridin-3-yl)-3- (trifluoromethyl)-1H- pyrazol-1-yl)propan-2-yl)- 3-(1-(methyl-d3)-1H- pyrazol-4-yl)propanamide | 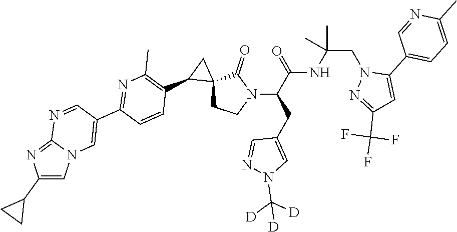 [SMILES: [2H]C([2H])([2H])n1cc(C[C@H](C(=O)NC(C)(C)Cn2nc(C(F)(F)F)cc2-c2ccc(C)nc2)N2CC[C@@]3(C[C@@H]3c3ccc(-c4cnc5nc(C6CC6)cn5c4)nc3C)C2=O)cn1] | 795.6 (M + H) |
| 176^{f} | (R)-3-(1-(difluoromethyl)- 1H-pyrazol-4-yl)-2- ((1R,3R)-1-(6-(2- (methoxymethyl)imidazo [1,2-a]pyrimidin-6-yl)-2- methylpyridin-3-yl)-4-oxo- 5-azaspiro[2.4]heptan-5- yl)-N-(2-methyl-1-(5-(6- methylpyrazin-2-yl)-3- (trifluoromethyl)-1H- pyrazol-1-yl)propan-2- yl)propanamide | 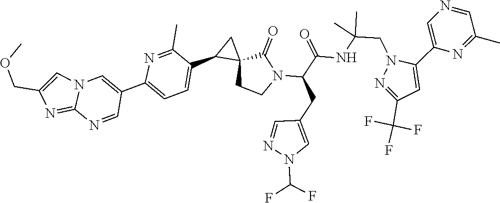 [SMILES: COCc1cn2cc(-c3ccc([C@H]4C[C@@]45CCN([C@H](Cc4cnn(C(F)F)c4)C(=O)NC(C)(C)Cn4nc(C(F)(F)F)cc4-c4cncc(C)n4)C5=O)c(C)n3)cnc2n1] | 833.2 (M + H) |
| 177^{g} | (R)-2-((1S,3R)-1-(2- chloro-4-(2- methylimidazo[1,2- a]pyrimidin-6-yl)phenyl)- 4-oxo-5- azaspiro[2.4]heptan-5-yl)- 3-(1-(difluoromethyl)-1H- pyrazol-4-yl)-N-(2-methyl- 1-(1-(1-methyl-2- oxopyrrolidin-3-yl)-4- (trifluoromethyl)-1H- imidazol-2-yl)propan-2- yl)propanamide (Isomer 1) | 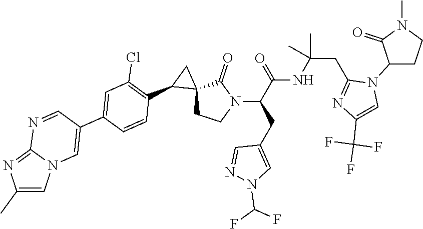 [SMILES: Cc1cn2cc(-c3ccc([C@H]4C[C@@]45CCN([C@H](Cc4cnn(C(F)F)c4)C(=O)NC(C)(C)Cc4nc(C(F)(F)F)cn4C4CCN(C)C4=O)C5=O)c(Cl)c3)cnc2n1] | 827.4 (M + H) |
| 178^{h} | (R)-3-(1-(difluoromethyl)- 1H-pyrazol-4-yl)-N-(1-(5- (6-(2-hydroxypropan-2- yl)pyridin-3-yl)-3- (trifluoromethyl)-1H- pyrazol-1-yl)-2- methylpropan-2-yl)-2- ((1R,3R)-1-(2-methyl-6-(2- methylimidazo[1,2- a]pyrimidin-6-yl)pyridin-3- yl)-4-oxo-5- azaspiro[2.4]heptan-5- yl)propanamide | 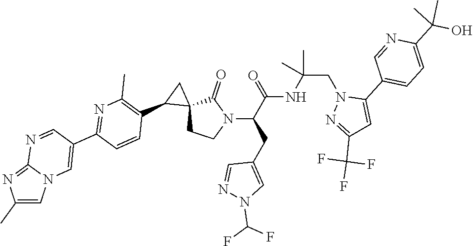 [SMILES: Cc1cn2cc(-c3ccc([C@H]4C[C@@]45CCN([C@H](Cc4cnn(C(F)F)c4)C(=O)NC(C)(C)Cn4nc(C(F)(F)F)cc4-c4ccc(C(C)(C)O)nc4)C5=O)c(C)n3)cnc2n1] | 846.6 (M + H) |
| 179ⁱ | (R)-2-((1R,3R)-1-(6-(2- cyclopropyl-2H- pyrazolo[3,4-b]pyridin-5- yl)-2-methylpyridin-3-yl)- 4-oxo-5- azaspiro[2.4]heptan-5-yl)- 3-(1-(difluoromethyl)-1H- pyrazol-4-yl)-N-(2-methyl- 1-(5-(2-methylpyrimidin-5- yl)-3-(trifluoromethyl)-1H- pyrazol-1-yl)propan-2- yl)propanamide | 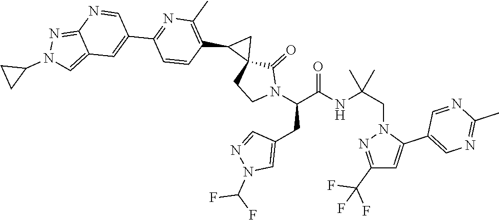 [SMILES: Cc1ncc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nn(C6CC6)cc5c4)nc3C)C2=O)cn1] | 829.3 (M + H) |
| 180 | (R)-3-(1-(difluoromethyl)- 1H-pyrazol-4-yl)-N-(1-(5- (3-methoxy-6- methylpyridazin-4-yl)-3- (trifluoromethyl)-1H- pyrazol-1-yl)-2- methylpropan-2-yl)-2- ((1R,3R)-1-(2-methyl-6-(2- methyl-2H-pyrazolo[3,4- b]pyridin-5-yl)pyridin-3- yl)-4-oxo-5- azaspiro[2.4]heptan-5- yl)propanamide | 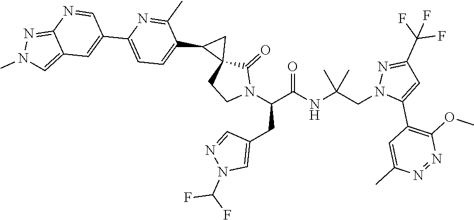 | 833.5 (M + H) |

^{a}3 equiv of 6-(2-hydroxypropan-2-yl)pyridine-3-boronic acid pinacol ester (95.0 mg, 2.68 eq, 361 μmol) were used. Reaction was stirred overnight and then a second addition of all reagents relative to chloropyridyl starting material was added.
^{b}3 equiv of 2-Methoxy-5-pyridinyl boronic acid were used. Reaction was stirred overnight.
^{c}3.5 equiv of 2-Methoxypyrimidine-5-boronic acid pinacol ester and 3.8 equiv of Cs₂CO₃ were used. Reaction was stirred overnight.
^{d}3 equiv of (5-fluoro-6-methoxypyridin-3-yl)boronic acid were used. Reaction was stirred overnight.
^{e}Reaction was stirred at 90° C. overnight.
^{f}2.5 equiv of (2-(methoxymethyl)imidazo[1,2-a]pyrimidin-6-yl)boronic acid, 0.074 equiv of Pd(dppf)Cl₂•CH₂Cl₂, and 5:1 1,4-dioxane:water were used.
^{g}2 equiv of (2-methylimidazo[1,2-a]pyrimidin-6-yl)boronic acid and 6:1 1,4-dioxane water was used. Purified by preparative HPLC (XSelect CSH C18, 50 mm × 250 mm, 5 μm, 100 mL/min) using a gradient of 5-95% ACN in water (0.1% aq. FA) to obtain the title compound as the first eluting isomer.
^{h}2.8 equiv of (2-methylimidazo[1,2-a]pyrimidin-6-yl)boronic acid and 4:1 1,4-dioxane:water was used.
ⁱBis[tris(tert-butyl)phosphine]palladium (0.2 equiv) and sodium carbonate (2 equiv) were used in place of Pd(dppf)Cl₂•CH₂Cl₂ and Cs₂CO₃ respectively. 10:1 1,4-dioxane:water was used, stirring at 60° C. for 1 h. The product was purified by silica gel chromatography using 5% MeOH in DCM; then re-purified by chiral SFC (column: Daicel Chiralcel OX 250 mm × 30 mm, 10 μm; mobile phase: 55% EtOH (+0.1% NH₃•H₂O) in CO₂) to remove a minor impurity.

### Example 181

### Methyl (5-((1S,2S)-2-(((R)-3-(1-(difluoromethyl)-1H-pyrazol-4-yl)-1-((1-(5-(5-fluoro-1-methyl-6-oxo-1,6-dihydropyridin-3-yl)-3-(trifluoromethyl)-1H-pyrazol-1-yl)-2-methylpropan-2-yl)amino)-1-oxopropan-2-yl) carbamoyl)cyclopropyl)-4-fluoro-2-hydroxyphenyl)(methyl)carbamate

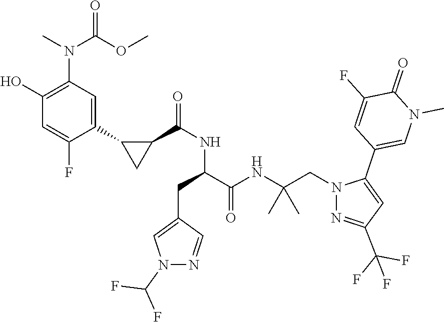 [SMILES: COC(=O)N(C)c1cc([C@H]2C[C@@H]2C(=O)N[C@H](Cc2cnn(C(F)F)c2)C(=O)NC(C)(C)Cn2nc(C(F)(F)F)cc2-c2cc(F)c(=O)n(C)c2)c(F)cc1O]

To (1S,2S)-2-(2-fluoro-4-hydroxy-5 ((methoxycarbonyl)(methyl)amino)phenyl)cyclopropane-1-carboxylic acid (173.1 mg, 611.1 μmol) was added HATU (476.4 mg, 1.25 mmol), DIPEA (400 μL, 2.30 mmol) and DMF (4 mL). The suspension was stirred at RT for 5 mins and then (R)-2-amino-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)propanamide (322 mg, 619.1 μmol) was added and stirred at RT for 1 h, partitioned between 15 mL water and 15 mL EtOAc, and the aqueous layer was extracted with additional EtOAc (15 mL×2). The combined organics were washed with sat. aq. NaCl (15 mL×3), dried over Na₂SO₄, filtered, and concentrated under reduced pressure. Flash chromatography (SiO₂, gradient 0-100% 3:1 EtOAc:EtOH in hexane), followed by preparative HPLC (C18 CSH, 30×250 mm, gradient 40-95% ACN in 0.1% aq. FA) provided the title compound (38.9 mg, 49 μmol, 8%) as a white powder. ES-MS m/z 785.4 (M+H).

The compounds in the following table were prepared as described in Example 181 using the appropriate carboxylic acid. Reactants can be added in different orders or in differing equivalency and differing methods can be used to work up or purify the compounds (normal phase, or high, neutral or low pH reversed phase). Such variances would be apparent to one skilled in the art.

TABLE 50

| Ex- | | | ES-MS |
|---|---|---|---|
| ample | Chemical Name | Structure | m/z |
| 182 | (R)-N-(1-(5-(1- cyclopropyl-2-oxo-1,2- dihydropyridin-4-yl)-3- (trifluoromethyl)-1H- pyrazol-1-yl)-2- methylpropan-2-yl)-3-(1- (difluoromethyl)-1H- pyrazol-4-yl)-2-((1R,3R)- 1-(6′-(2-hydroxypropan-2- yl)-6-methyl-[2,3′- bipyridin]-5-yl)-4-oxo-5- azaspiro[2.4]heptan-5- yl)propanamide | 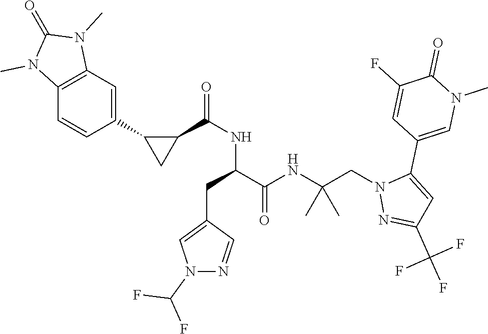 | 748.4 (M + H) |
| 183 | (1S,2S)-2-(3-chloro-1- methyl-1H-indazol-6-yl)- N-((R)-3-(1- (difluoromethyl)-1H- pyrazol-4-yl)-1-((1-(5-(5- fluoro-1-methyl-6-oxo-1,6- dihydropyridin-3-yl)-3- (trifluoromethyl)-1H- pyrazol-1-yl)-2- methylpropan-2-yl)amino)- 1-oxopropan-2- yl)cyclopropane-1- carboxamide | 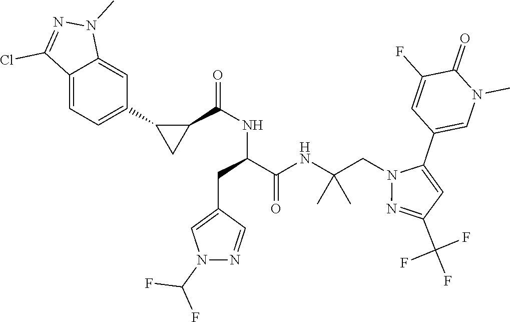 [SMILES: Cn1cc(-c2cc(C(F)(F)F)nn2CC(C)(C)NC(=O)[C@@H](Cc2cnn(C(F)F)c2)NC(=O)[C@H]2C[C@@H]2c2ccc3c(Cl)nn(C)c3c2)cc(F)c1=O] | 752.2 (M + H) |

### Example 184

### (R)-3-(1-(difluoromethyl)-1H-pyrazol-4-yl)-N-(1-(5-(5-fluoro-1-methyl-6-oxo-1,6-dihydropyridin-3-yl)-3-(trifluoromethyl)-1H-pyrazol-1-yl)-2-methylpropan-2-yl)-2-((trans)-1-(1-methyl-1H-indazol-6-yl)-4-oxo-5-azaspiro[2.4]heptan-5-yl)propanamide

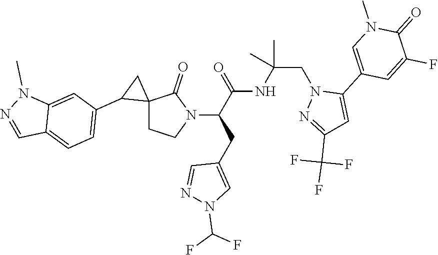 [SMILES: Cn1cc(-c2cc(C(F)(F)F)nn2CC(C)(C)NC(=O)[C@@H](Cc2cnn(C(F)F)c2)N2CCC3(CC3c3ccc4cnn(C)c4c3)C2=O)cc(F)c1=O]

To (trans)-1-(2-chloroethyl)-N—((R)-3-(1-(difluoromethyl)-1H-pyrazol-4-yl)-1-((1-(3-(5-fluoro-1-methyl-6-oxo-1,6-dihydropyridin-3-yl)-5-(trifluoromethyl)-1H-pyrazol-1-yl)-2-methylpropan-2-yl)amino)-1-oxopropan-2-yl)-2-(1-methyl-1H-indazol-6-yl)cyclopropane-1-carboxamide (501.8 mg, 611.0 μmol) in NMP (3 mL) was added Cs₂CO₃ (461.8 mg, 1.42 mmol). The resulting mixture was stirred vigorously at RT for 1 h. The crude mixture was filtered through a 0.45 μm syringe filter and purified by preparative HPLC (XSelect CSH C18, 50×250 mm, 5 μm, 100 mL/min) using a gradient of 5-95% ACN/water (w/10 mM NH₄OAc) and chiral SFC purification (Chiralpak OX-H 30×250 mm×5 μM, 34 mL/min), eluting with MeOH to afford the title compound (96.2 mg, 21%) as the second eluting diastereomer. ES-MS m/z 744.4 (M+H)

### Example 185

### (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide

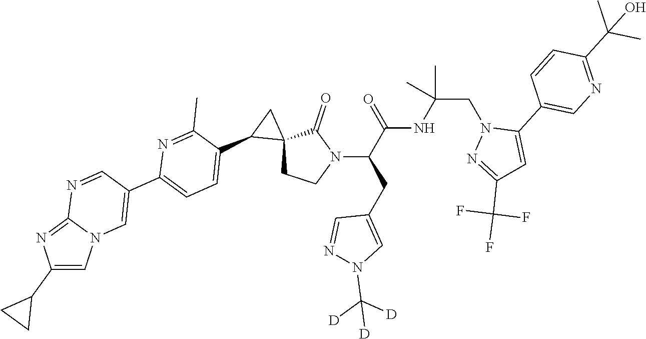 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(-c4cnc5nc(C6CC6)cn5c4)nc3C)C2=O)cn1]

To sodium carbonate (40 mg, 0.38 mmol), was added (2-cyclopropylimidazo[1,2-a]pyrimidin-6-yl) boronic acid (36.7 mg, 181 μmol), (A-1ªPhos) 2PdCl₂ (5.2 mg, 7.3 μmol), and a solution of (R)-2-((1R,3R)-1-(6-chloro-2-methylpyridin-3-yl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide (98 mg, 0.14 mmol) in MeOH (1.0 mL). The vial was flushed with Ar, capped, and stirred at 65° C. overnight. The mixture was cooled to RT, and more (A-1ªPhos) 2PdCl₂ (5.2 mg, 7.3 μmol), sodium carbonate (40 mg, 0.38 mmol), and (2-cyclopropylimidazo[1,2-a]pyrimidin-6-yl) boronic acid (36.7 mg, 181 μmol) were added and the mixture was stirred at 65° C. under Ar for 1 h, then cooled to RT. The reaction mixture was treated with SiliaMetS Thiol resin (100 mg) then stirred at RT for 1 h, preparative HPLC (XSelect CSH C18, 50×250 mm, 5 μm, gradient 5-95% ACN with 10 mM NH₄OAc, 100 mL/min) to afford the title compound (43.4 mg, 38%) as a white solid. ES-MS m/z 839.4 (M+H).

The compounds in the following table were prepared as described in Example 185 using the appropriate arylboronic acid and aryl halide. Reactants can be added in different orders or in differing equivalency and differing methods can be used to work up or purify the compounds (normal phase, or high, neutral or low pH reversed phase). Such variances would be apparent to one skilled in the art.

TABLE 51

| Ex- | | | ES-MS |
|---|---|---|---|
| ample | Chemical Name | Structure | m/z |
| 186 | (R)-2-((1R,3R)-1-(6-(2- cyclopropylimidazo[1,2- a]pyrimidin-6-yl)-2- methylpyridin-3-yl)-4-oxo- 5-azaspiro[2.4]heptan-5- yl)-N-(1-(5-(2-(2- hydroxypropan-2- yl)pyrimidin-5-yl)-3- (trifluoromethyl)-1H- pyrazol-1-yl)-2- methylpropan-2-yl)-3-(1- (methyl-d3)-1H-pyrazol-4- yl)propanamide | 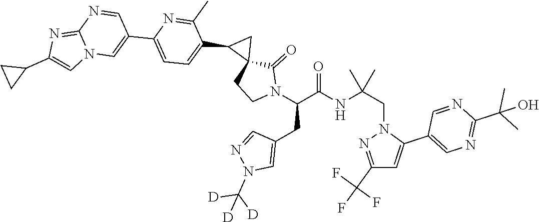 [SMILES: [2H]C([2H])([2H])n1cc(C[C@H](C(=O)NC(C)(C)Cn2nc(C(F)(F)F)cc2-c2cnc(C(C)(C)O)nc2)N2CC[C@@]3(C[C@@H]3c3ccc(-c4cnc5nc(C6CC6)cn5c4)nc3C)C2=O)cn1] | 840.4 (M + H) |
| 187 | (R)-2-((1S,3R)-1-(2- chloro-4-(2- methylimidazo[1,2- a]pyrimidin-6-yl)phenyl)- 4-oxo-5- azaspiro[2.4]heptan-5-yl)- N-(1-(5-(2-(2- hydroxypropan-2- yl)pyrimidin-5-yl)-3- (trifluoromethyl)-1H- pyrazol-1-yl)-2- methylpropan-2-yl)-3-(1- (methyl-d3)-1H-pyrazol-4- yl)propanamide | 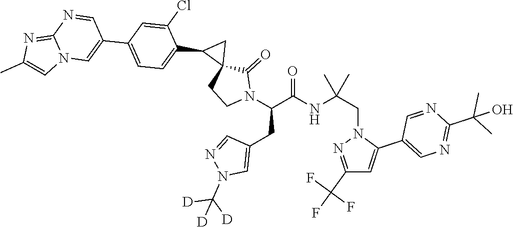 [SMILES: [2H]C([2H])([2H])n1cc(C[C@H](C(=O)NC(C)(C)Cn2nc(C(F)(F)F)cc2-c2cnc(C(C)(C)O)nc2)N2CC[C@@]3(C[C@@H]3c3ccc(-c4cnc5nc(C)cn5c4)cc3Cl)C2=O)cn1] | 833.4 (M + H) |
| 188 | (R)-2-((1S,3R)-1-(2- chloro-4-(2- (methoxymethyl)imidazo [1,2-a]pyrimidin-6- yl)phenyl)-4-oxo-5- azaspiro[2.4]heptan-5-yl)- N-(1-(5-(6-(2- hydroxypropan-2- yl)pyridin-3-yl)-3- (trifluoromethyl)-1H- pyrazol-1-yl)-2- methylpropan-2-yl)-3-(1- (methyl-d3)-1H-pyrazol-4- yl)propanamide | 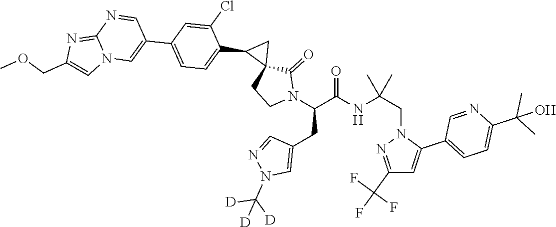 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(-c4cnc5nc(COC)cn5c4)cc3Cl)C2=O)cn1] | 862.4 (M + H) |

### Example 189

### (R)-2-((1R,3R)-1-(6-(2-Ethyl-2H-pyrazolo[3,4-b]pyridin-5-yl)-2-methylpyridin-3-yl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide

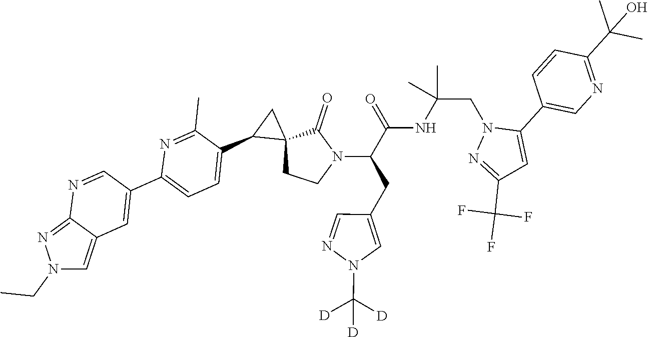 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(-c4cnc5nn(CC)cc5c4)nc3C)C2=O)cn1]

A solution of 2-ethyl-5-(4,4,5,5-tetramethyl-1,3,2-dioxaborolan-2-yl)-2H-pyrazolo[3,4-b]pyridine (78 mg, 0.29 mmol) in 1,4-dioxane (1.5 mL) was charged with PdCl₂(dppf)·CH₂Cl₂ (9 mg, 0.01 mmol), Cs₂CO₃ (250 mg, 767 μmol), water (0.25 mL), and a solution of (R)-2-((1R,3R)-1-(6-chloro-2-methylpyridin-3-yl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide (98 mg, 0.14 mmol) in MeOH (1 mL). The vial headspace was flushed with Ar, then the reaction mixture was stirred at 60° C. under Ar overnight. The reaction mixture was cooled to RT and treated with SiliaMetS thiol resin (300 mg), stirred at RT for 1 h, and filtered, rinsing with ACN. The filtrate was concentrated at 40° C. under N₂, resuspended in ACN, and refiltered. The filtrate was concentrated and the residue was purified by reverse-phase HPLC (XSelect CSH C18, 50×250 mm, 5 μm, gradient 5-95% ACN in 0.1% aq. FA, 100 mL/min) to provide the title compound (44.5 mg, 39%) as a white solid. ES-MS m/z 827.4 (M+H).

The compounds in the following table were prepared as described in Example 189 using the appropriate arylboronic acid and aryl halide. Reactants can be added in different orders or in differing equivalency and differing methods can be used to work up or purify the compounds (normal phase, or high, neutral or low pH reversed phase). Such variances would be apparent to one skilled in the art.

TABLE 52

| Ex- | | | ES-MS |
|---|---|---|---|
| ample | Chemical Name | Structure | m/z |
| 190^{a} | (R)-2-((1R,3R)-1-(6-(2- cyclopropyl-2H- pyrazolo[3,4-b]pyridin-5- yl)-2-methylpyridin-3-yl)- 4-oxo-5- azaspiro[2.4]heptan-5-yl)- N-(1-(5-(6-(2- hydroxypropan-2- yl)pyridin-3-yl)-3- (trifluoromethyl)-1H- pyrazol-1-yl)-2- methylpropan-2-yl)-3-(1- (methyl-d3)-1H-pyrazol-4- yl)propanamide | 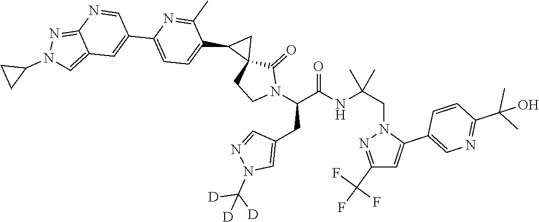 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(-c4cnc5nn(C6CC6)cc5c4)nc3C)C2=O)cn1] | 839.6 (M + H) |
| 191^{b} | (R)-2-((1R,3R)-1-(6-(2- cyclopropyl-2H- pyrazolo[3,4-b]pyridin-5- yl)-2-methylpyridin-3-yl)- 4-oxo-5- azaspiro[2.4]heptan-5-yl)- N-(1-(5-(2-(2- hydroxypropan-2- yl)pyrimidin-5-yl)-3- (trifluoromethyl)-1H- pyrazol-1-yl)-2- methylpropan-2-yl)-3-(1- (methyl-d3)-1H-pyrazol-4- yl)propanamide | 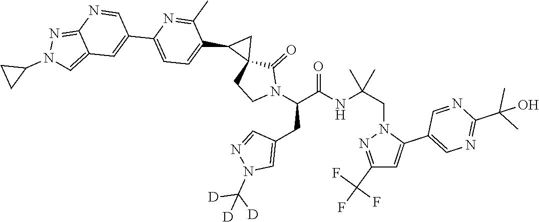 [SMILES: [2H]C([2H])([2H])n1cc(C[C@H](C(=O)NC(C)(C)Cn2nc(C(F)(F)F)cc2-c2cnc(C(C)(C)O)nc2)N2CC[C@@]3(C[C@@H]3c3ccc(-c4cnc5nn(C6CC6)cc5c4)nc3C)C2=O)cn1] | 840.4 (M + H) |
| 192^{c} | (R)-2-((1R,3R)-1-(2- cyclopropyl-6-(2- (methoxymethyl)imidazo [1,2-a]pyrimidin-6- yl)pyridin-3-yl)-4-oxo-5- azaspiro[2.4]heptan-5-yl)- N-(1-(5-(6-(2- hydroxypropan-2- yl)pyridin-3-yl)-3- (trifluoromethyl)-1H- pyrazol-1-yl)-2- methylpropan-2-yl)-3-(1- (methyl-d3)-1H-pyrazol-4- yl)propanamide | 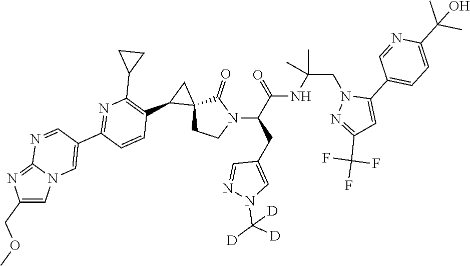 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(-c4cnc5nc(COC)cn5c4)nc3C3CC3)C2=O)cn1] | 869.4 (M + H) |

^{a}Mobile phase additive: 10 mM NH₄OAc
^{b}(CSH, 30 × 250 mm, gradient 5-95% ACN in 0.1% aq. FA, 40 mL/min)
^{c}(CSH, 50 × 250 mm, gradient 5-95% ACN with 10 mM NH₄OAc, 100 mL/min)

### Example 193

### (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide

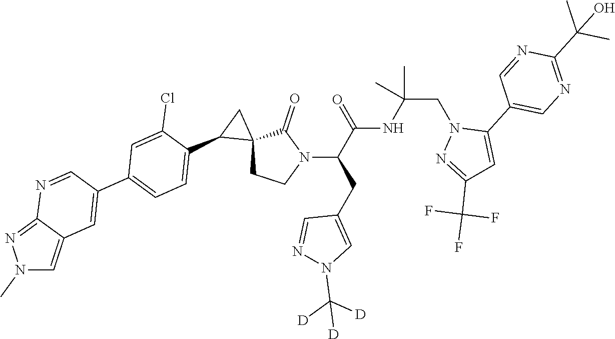 [SMILES: [2H]C([2H])([2H])n1cc(C[C@H](C(=O)NC(C)(C)Cn2nc(C(F)(F)F)cc2-c2cnc(C(C)(C)O)nc2)N2CC[C@@]3(C[C@@H]3c3ccc(-c4cnc5nn(C)cc5c4)cc3Cl)C2=O)cn1]

A mixture of (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(methyl-d3)-1H-pyrazol-4-yl)propanoic acid (101.8 mg, 200 μmol), 2-(5-(1-(2-amino-2-methylpropyl)-3-(trifluoromethyl)-1H-pyrazol-5-yl)pyrimidin-2-yl)propan-2-ol hydrochloride (105 mg, 260 μmol), N-methylmorpholine (100 μL, 910 μmol), and DMA (1.0 mL) was treated with DEPBT (120 mg, 401 μmol) and stirred at RT overnight. The reaction mixture was filtered through a 0.45 μm PTFE syringe filter and purified by reverse-phase HPLC (XSelect CSH C18, 50×250 mm, 5 μm, gradient 5-95% ACN in 10 mM aq. NH₄OAc) to provide the title compound (134.2 mg, 80%) as a white solid. ES-MS m/z 833.4/835.4 (M+H) (Cl^{35/37}).

### Example 194

### (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide

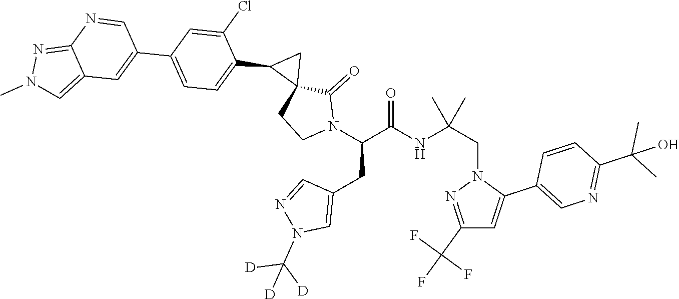 [SMILES: [2H]C([2H])([2H])n1cc(C[C@H](C(=O)NC(C)(C)Cn2nc(C(F)(F)F)cc2-c2ccc(C(C)(C)O)nc2)N2CC[C@@]3(C[C@@H]3c3ccc(-c4cnc5nn(C)cc5c4)cc3Cl)C2=O)cn1]

To 2-(5-(1-(2-amino-2-methylpropyl)-3-(trifluoromethyl)-1H-pyrazol-5-yl)pyridin-2-yl)propan-2-ol dihydrochloride (1.00 g, 2.36 mmol) was added (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(methyl-d3)-1H-pyrazol-4-yl)propanoic acid (1.02 g, 1.96 mmol) in DMA (5.0 mL). The mixture was stirred at RT, and N-methylmorpholine (1.3 mL, 12 mmol) was added, followed by DEPBT (1.2 g, 4.0 mmol). The resulting suspension was stirred at RT under Ar overnight, diluted with water (5 mL), and purified by reverse-phase flash chromatography (C18, gradient 10-100% ACN in 10 mM aq. NH₄HCO₃₊₅% MeOH) to provide the title compound (1.136 g, 69%) as a white powder. ES-MS m/z 832 (M+H).

### Preparation 462

### Diallyl (2-(5-(1-(2-((R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)propanamido)-2-methylpropyl)-3-(trifluoromethyl)-1H-pyrazol-5-yl)pyrimidin-2-yl)propan-2-yl)phosphate

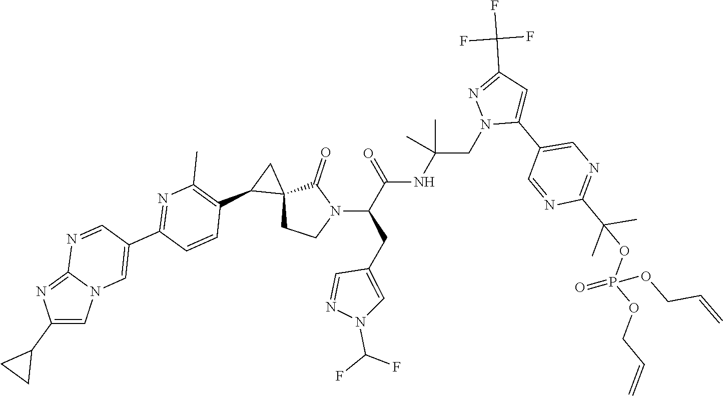 [SMILES: C=CCOP(=O)(OCC=C)OC(C)(C)c1ncc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nc(C6CC6)cn5c4)nc3C)C2=O)cn1]

A 40 mL vial containing a magnetic stirbar and (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)-N-(1-(5-(2-(2-hydroxypropan-2-yl)pyrimidin-5-yl)-3-(trifluoromethyl)-1H-pyrazol-1-yl)-2-methylpropan-2-yl)propanamide (540 mg, 94% purity, 582 μmol) was charged with 4 Å molecular sieves (250 mg) and a solution of tetrazole in ACN (2.5 mL, 0.45 M, 1.1 mmol). The reaction mixture was stirred at 0° C. under Ar as a solution of diallyl-N,N-diisopropylphosphoramidite (300 μL, 1.13 mmol) was added. The reaction mixture was stirred at 0° C. for 10 min, then 35% aq. H₂O₂ (600 μL, 6.8 mmol) was added. The reaction mixture was stirred at RT for 10 min, then loaded directly onto diatomaceous earth. Reverse-phase flash chromatography (C18, gradient 50-70% ACN in 10 mM aq. NH₄HCO₃ with 5% MeOH) provided the title compound (459 mg, 76%) as a thick yellow oil. ES-MS m/z 1033 (M+H).

### Preparation 463

### Diallyl (2-(5-(1-(2-((R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(methyl-d3)-1H-pyrazol-4-yl)propanamido)-2-methylpropyl)-3-(trifluoromethyl)-1H-pyrazol-5-yl)pyrimidin-2-yl)propan-2-yl)phosphate

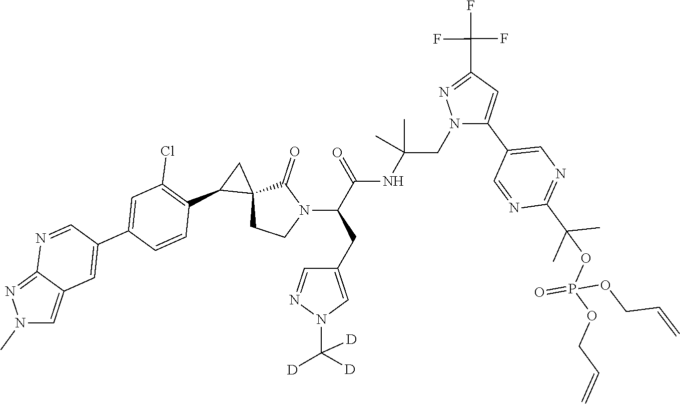 [SMILES: [2H]C([2H])([2H])n1cc(C[C@H](C(=O)NC(C)(C)Cn2nc(C(F)(F)F)cc2-c2cnc(C(C)(C)OP(=O)(OCC=C)OCC=C)nc2)N2CC[C@@]3(C[C@@H]3c3ccc(-c4cnc5nn(C)cc5c4)cc3Cl)C2=O)cn1]

The title compound was prepared as described in Preparation 462 using (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-N-(1-(5-(2-(2-hydroxypropan-2-yl)pyrimidin-5-yl)-3-(trifluoromethyl)-1H-pyrazol-1-yl)-2-methylpropan-2-yl)-3-(1-(methyl-d3)-1H-pyrazol-4-yl)propanamide. ES-MS m/z 993 (M+H).

### Preparation 464

### Diallyl (2-(5-(1-(2-((R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)propanamido)-2-methylpropyl)-3-(trifluoromethyl)-1H-pyrazol-5-yl)pyridin-2-yl)propan-2-yl)

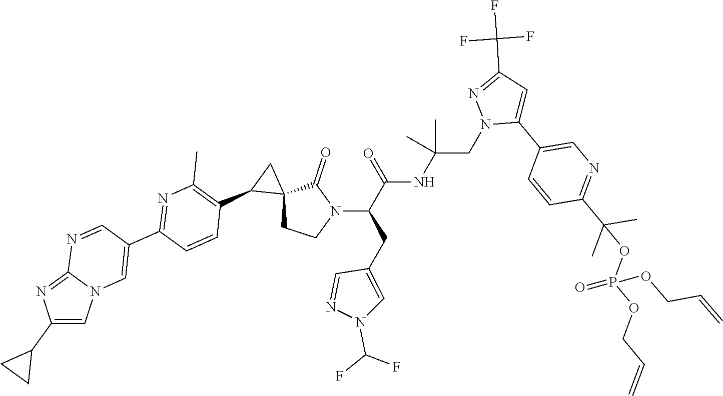 [SMILES: C=CCOP(=O)(OCC=C)OC(C)(C)c1ccc(-c2cc(C(F)(F)F)nn2CC(C)(C)NC(=O)[C@@H](Cc2cnn(C(F)F)c2)N2CC[C@@]3(C[C@@H]3c3ccc(-c4cnc5nc(C6CC6)cn5c4)nc3C)C2=O)cn1]

The title compound was prepared as described in Preparation 462 using (R)-2-((1R,3R)-1-(6-(2-cyclopropylimidazo[1,2-a]pyrimidin-6-yl)-2-methylpyridin-3-yl)-4-oxo-5-azaspiro[2.4]heptan-5-yl)-3-(1-(difluoromethyl)-1H-pyrazol-4-yl)-N-(1-(5-(6-(2-hydroxypropan-2-yl)pyridin-3-yl)-3-(trifluoromethyl)-1H-pyrazol-1-yl)-2-methylpropan-2-yl)propanamide. ES-MS m/z 1032 (M+H).

### Preparation 465

### Diallyl (2-(5-(1-(2-((R)-2-((1R,3R)-1-(2-cyclopropyl-6-(2-methoxymethyl) imidazo[1,2-a]pyrimidin-6-yl)pyridin-3-yl)-4-oxo-5-azaspiro[2.4]heptan-5-yl)-3-(1-(methyl-d3)-1H-pyrazol-4-yl)propanamido)-2-methylpropyl)-3-(trifluoromethyl)-1H-pyrazol-5-yl)pyridin-2-yl)propan-2-yl)phosphate

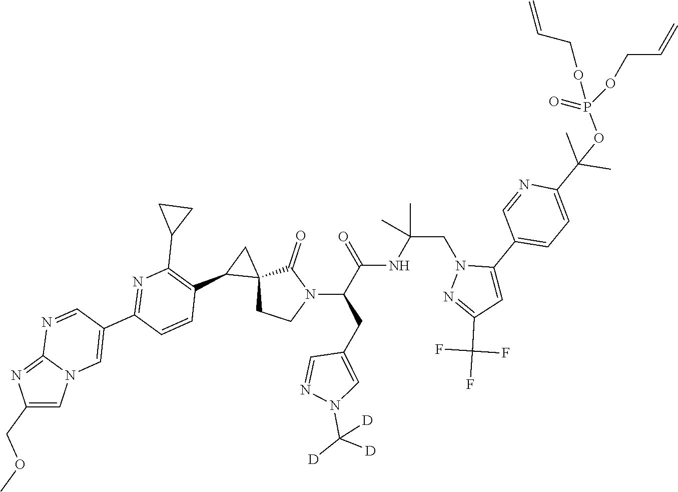 [SMILES: [2H]C([2H])([2H])n1cc(C[C@H](C(=O)NC(C)(C)Cn2nc(C(F)(F)F)cc2-c2ccc(C(C)(C)OP(=O)(OCC=C)OCC=C)nc2)N2CC[C@@]3(C[C@@H]3c3ccc(-c4cnc5nc(COC)cn5c4)nc3C3CC3)C2=O)cn1]

The title compound may be prepared as described in Preparation 462 using (R)-2-((1R,3R)-1-(2-cyclopropyl-6-(2-(methoxymethyl) imidazo[1,2-a]pyrimidin-6-yl)pyridin-3-yl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide.

### Preparation 466

### Diallyl (2-(5-(1-(2-((R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(methyl-d3)-1H-pyrazol-4-yl)propanamido)-2-methylpropyl)-3-(trifluoromethyl)-1H-pyrazol-5-yl)pyridin-2-yl)propan-2-yl)phosphate

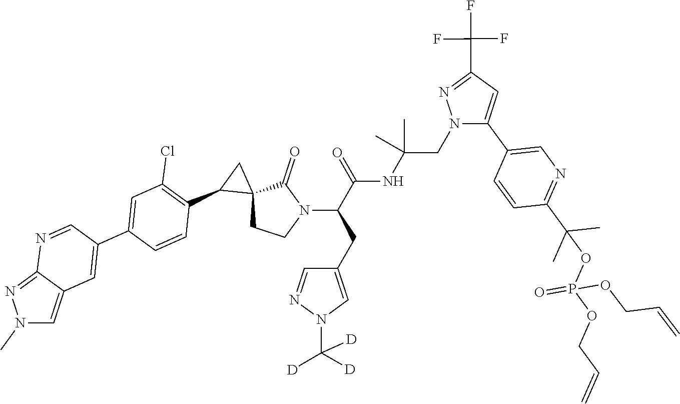 [SMILES: [2H]C([2H])([2H])n1cc(C[C@H](C(=O)NC(C)(C)Cn2nc(C(F)(F)F)cc2-c2ccc(C(C)(C)OP(=O)(OCC=C)OCC=C)nc2)N2CC[C@@]3(C[C@@H]3c3ccc(-c4cnc5nn(C)cc5c4)cc3Cl)C2=O)cn1]

A vial was charged with a magnetic stirbar, (R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-N-(1-(5-(6-(2-hydroxypropan-2-yl)pyridin-3-yl)-3-(trifluoromethyl)-1H-pyrazol-1-yl)-2-methylpropan-2-yl)-3-(1-(methyl-d3)-1H-pyrazol-4-yl)propanamide (283 mg, 340 μmol), and 4 Å molecular sieves (100 mg). A solution of tetrazole in ACN (1.7 mL, 0.45 molar, 0.77 mmol) was added, and the reaction mixture was stirred at 0° C. under argon. Diallyl-N,N-diisopropylphosphoramidite (200 μL, 757 μmol) was added, the reaction mixture was stirred at 0° C. for 10 min, then anhydrous tert-butyl hydroperoxide in decane (400 μL, 5.5 M, 2.20 mmol) was added. The reaction mixture was stirred at RT for 1.5 h, then combined with another batch run as described above (243 μmol scale) and loaded onto diatomaceous earth. Reverse-phase flash chromatography (C18, gradient 0-100% ACN in 10 mM aq. NH₄HCO₃ with 5% MeOH) provided the title compound (395.7 mg, 68%) as a yellow oil. ES-MS m/z 992 (M+H).

### Example 195

### 2-(5-(1-(2-((R)-2-((1S,3R)-1-(2-Chloro-4-(2-methyl-2H-pyrazolo[3,4-b]pyridin-5-yl)phenyl)-4-oxo-5-azaspiro[2.4]heptan-5-yl)-3-(1-(methyl-d3)-1H-pyrazol-4-yl)propanamido)-2-methylpropyl)-3-(trifluoromethyl)-1H-pyrazol-5-yl)pyridin-2-yl)propan-2-yl dihydrogen phosphate, ammonia salt

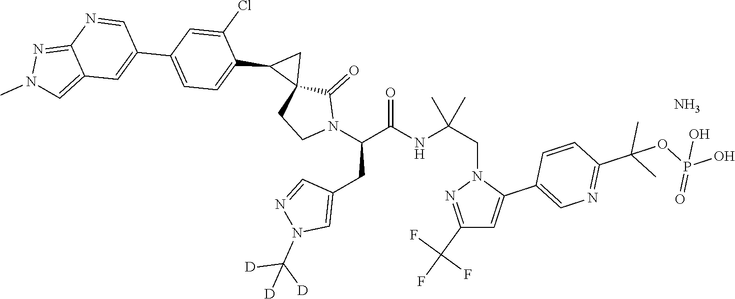

A vial was charged with a magnetic stirbar, diallyl (2-(5-(1-(2-((R)-2-((1S,3R)-1-(2-chloro-4-(2-methyl-2H-pyrazolo[3,4-b]pyridin-5-yl)phenyl)-4-oxo-5-azaspiro[2.4]heptan-5-yl)-3-(1-(methyl-d3)-1H-pyrazol-4-yl)propanamido)-2-methylpropyl)-3-(trifluoromethyl)-1H-pyrazol-5-yl)pyridin-2-yl)propan-2-yl)phosphate (395 mg, 398 μmol), and CH₂Cl₂ (4 mL), flushed with Ar, capped, and sparged with Ar at RT for 5 min, cooled to 0° C., and phenylsilane (150 μL, 1.21 mmol) was added, followed by Pd(PPh₃)₄ (25.0 mg, 21.6 μmol). The reaction mixture was stirred at 0° C. for 1 h, treated with a thiol resin (Silicycle SiliaMetS, 1.5 mmol/g, 270 mg), and concentrated at RT under argon. Reverse-phase flash chromatography (C18, gradient 0-100% ACN in 10 mM aq. NH₄HCO₃ with 5% MeOH) provided the title compound (282.3 mg, 76%) as a white solid. ES-MS m/z 912 (M+H).

The compounds in the following table were prepared as described in Example 195 using the appropriate phosphate ester. Reactants can be added in different orders or in differing equivalency and differing methods can be used to work up or purify the compounds (normal phase, or high, neutral or low pH reversed phase). The reaction may be run in either CH₂Cl₂ or THF at temperatures between RT and 0° C. Such variances would be apparent to one skilled in the art.

TABLE 53

| Ex- | | | ES-MS |
|---|---|---|---|
| ample | Chemical Name | Structure | m/z |
| 196 | 2-(5-(1-(2-((R)-2-((1R,3R)-1- (6-(2-Cyclopropylimidazo[1,2- a]pyrimidin-6-yl)-2- methylpyridin-3-yl)-4-oxo-5- azaspiro[2.4]heptan-5-yl)-3-(1- (difluoromethyl)-1H-pyrazol-4- yl)propanamido)-2- methylpropyl)-3- (trifluoromethyl)-1H-pyrazol-5- yl)pyrimidin-2-yl)propan-2-yl dihydrogen phosphate, ammonia salt | 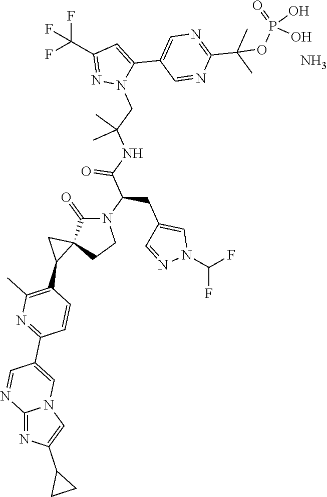 | 953 (M + H) |
| 197^{a} | 2-(5-(1-(2-((R)-2-((1R,3R)-1- (6-(2-Cyclopropylimidazo[1,2- a]pyrimidin-6-yl)-2- methylpyridin-3-yl)-4-oxo-5- azaspiro[2.4]heptan-5-yl)-3-(1- (difluoromethyl)-1H-pyrazol-4- yl)propanamido)-2- methylpropyl)-3- (trifluoromethyl)-1H-pyrazol-5- yl)pyridin-2-yl)propan-2-yl dihydrogen phosphate, ammonia salt | 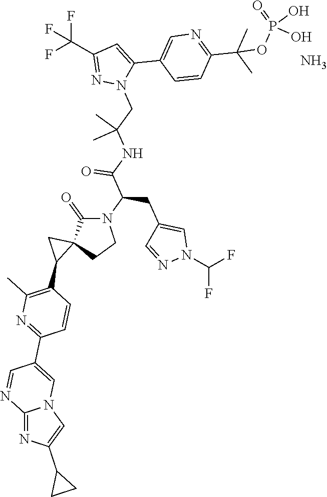 | 952 (M + H) |
| 198 | 2-(5-(1-(2-((R)-2-((1S,3R)-1-(2- Chloro-4-(2-methyl-2H- pyrazolo[3,4-b]pyridin-5- yl)phenyl)-4-oxo-5- azaspiro[2.4]heptan-5-yl)-3-(1- (methyl-d3)-1H-pyrazol-4- yl)propanamido)-2- methylpropyl)-3- (trifluoromethyl)-1H-pyrazol-5- yl)pyrimidin-2-yl)propan-2-yl dihydrogen phosphate, ammonia salt | 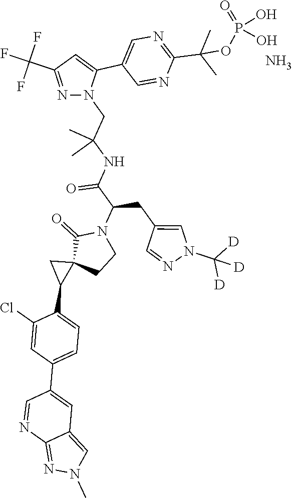 | 913 (M + H) |

^{a}Run in THF

The following compound may also be prepared as described in Example 195 using the appropriate phosphate ester. Reactants can be added in different orders or in differing equivalency and differing methods can be used to work up or purify the compounds (normal phase, or high, neutral or low pH reversed phase). The reaction may be run in either CH₂Cl₂ or THF at temperatures between RT and 0° C. Such variances would be apparent to one skilled in the art.

| | | | |
|---|---|---|---|
| 199 | 2-(5-(1-(2-((R)-2-((1R,3R)-1- (2-cyclopropyl-6-(2- (methoxymethyl)imidazo[1,2- a]pyrimidin-6-yl)pyridin-3-yl)- 4-oxo-5-azaspiro[2.4]heptan-5- yl)-3-(1-(methyl-d3)-1H- pyrazol-4-yl)propanamido)-2- methylpropyl)-3- (trifluoromethyl)-1H-pyrazol-5- yl)pyridin-2-yl)propan-2-yl dihydrogen phosphate, ammonia salt | 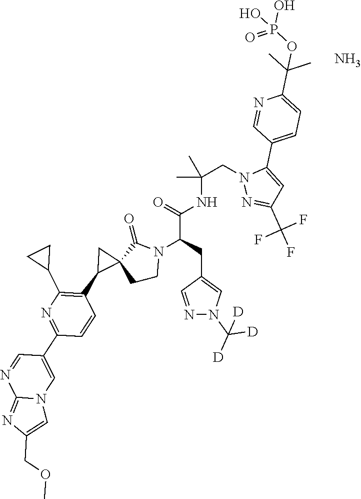 | 949.4 (M + H) |

Functional hGIP-R Assay

Functional activity was determined using CAMP formation in HEK-293 clonal cell lines expressing hGIP-R. hGIP-R receptor expressing cells were treated with compound (20 point concentration-response curve in DMSO, 2-fold Labcyte Echo direct dilution, 384 well plate Corning Cat #3570) in DMEM (Gibco Cat #31053) supplemented with 1× GlutaMAX™ (Gibco Cat #35050), 0.1% bovine casein (Sigma C₄₇₆₅-10ML), 250 μM IBMX (3-Isobutyl-1-methylxanthine, Acros Cat #228420010) and 20 mM HEPES (Gibco Cat #15630) in a 20 μL assay volume (final DMSO concentration was 1%). After a 30 min incubation at 37° C., the resulting increase in intracellular cAMP was quantitatively determined using the Revvity CAMP Dynamic 2 HTRF Assay Kit (62AM4PEJ). Briefly, cAMP levels within the cell were detected by adding the cAMP-d2 conjugate in cell lysis buffer (10 μL) followed by the antibody anti-cAMP-Eu³⁺-Cryptate, also in cell lysis buffer (10 μL). The resulting competitive assay was incubated for at least 60 min at RT, then detected using a BMG Labtech PHERAstar FSX instrument with excitation at 320 nm and emission at 665 nm and 620 nm. PHERAstar units (emission at 665 nm/620 nm*10,000) are inversely proportional to the amount of cAMP present and were converted to nM cAMP per well using a CAMP standard curve. The amount of cAMP generated (nM) in each well was converted to a percent of the maximal response observed with human GIP (1-42) OH. A relative EC₅₀ value and percent top (Emax) were derived by non-linear regression analysis using the percent maximal response vs. the concentration of compound added, fitted to a four-parameter logistic equation.

Results. Examples 7 and 11 to 198 were tested using the above-described assay and found to have an EC₅₀ of 300 nM or less. This data indicates that these compounds are agonists of the human GIPR. Functional data for particular exemplified compounds is in Table 55 below.

TABLE 55
Functional cAMP Potency (EC₅₀) and Efficacy (Eₘₐₓ) for compounds
incubated at 37° C. in the presence of 0.1% bovine casein.

| | |
|---|---|
| Example | Human GIP-R |

| No. | EC₅₀, nM (SEM, n) ^{a} | | Eₘₐₓ, % ^{b} |
|---|---|---|---|
| 7 | 246 | (24.1, n = 2) | 99.7 ± 0.756 |
| 11 | 55.2 | (2.51, n = 2) | 117 ± 5.22 |
| 12 | 104 | (12.6, n = 3) | 106 ± 5.38 |
| 13 | 49.7 | (4.96, n = 2) | 112 ± 0.275 |
| 14 | 87.7 | (21.7, n = 2) | 103 ± 10.2 |
| 15 | 24.6 | (4.04, n = 3) | 117 ± 6.6 |
| 16 | 36.0 | (0.248, n = 2) | 104 ± 4.17 |
| 17 | 83.5 | (0.942, n = 2) | 129 ± 11.4 |
| 18 | 12.0 | (1.80, n = 2) | 116 ± 13 |
| 19 | 66.9 | (8.31, n = 2) | 123 ± 1.72 |
| 20 | 79.7 | (17.3, n = 2) | 126 ± 2.39 |
| 21 | 63.4 | (0.933, n = 2) | 102 ± 1.71 |
| 22 | 61.3 | (5.34, n = 2) | 109 ± 11.6 |
| 23 | 44.4 | (2.37, n = 2) | 111 ± 11.6 |
| 24 | 15.8 | (2.56, n = 2) | 133 ± 0.579 |
| 25 | 82.7 | (14.1, n = 2) | 142 ± 17.3 |
| 26 | 17.4 | (2.25, n = 3) | 132 ± 15.5 |
| 27 | 61.6 | | 99.3 |
| 28 | 20.5 | (6.23, n = 2) | 91.9 ± 0.597 |
| 29 | 79.8 | | 99.4 |
| 30 | 198 | (11.6, n = 2) | 95.2 ± 0.936 |
| 31 | 152 | (3.58, n = 3) | 117 ± 4.92 |
| 32 | 93.5 | | 119 |
| 33 | 133 | | 108 |
| 34 | 183 | (37.6, n = 2) | 113 ± 12.3 |
| 35 | 135 | (22.9, n = 3) | 75 ± 4.1 |
| 36 | 104 | (30.9, n = 3) | 94 ± 11.1 |
| 37 | 10.8 | (1.26, n = 2) | 77.4 ± 5.92 |
| 38 | 7.93 | | 92.1 |
| 39 | 63.1 | (12.5, n = 2) | 77.8 ± 2.57 |
| 40 | 3.55 | (0.118, n = 2) | 60.7 ± 0.306 |
| 41 | 20.2 | (7.72, n = 2) | 111 ± 4.76 |
| 42 | 52.7 | (4.64, n = 2) | 114 ± 10.2 |
| 43 | 61.1 | (8.21, n = 3) | 116 ± 4.26 |
| 44 | 122 | (8.70, n = 2) | 111 ± 17.5 |
| 45 | 10.7 | (1.69, n = 3) | 79.8 ± 9.84 |
| 46 | 77.9 | (8.26, n = 4) | 66.4 ± 2.06 |
| 47 | 41.5 | (0.508, n = 2) | 84.6 ± 1.49 |
| 48 | 76.9 | (2.79, n = 2) | 56.5 ± 4.38 |
| 49 | 12.6 | (1.80, n = 2) | 72.6 ± 3.47 |
| 50 | 82.3 | (7.83, n = 3) | 85.5 ± 2.37 |
| 51 | 62.7 | (14.1, n = 2) | 120 ± 6.49 |
| 52 | 53.0 | (0.825, n = 2) | 98.4 ± 3.96 |
| 53 | 22.6 | (0.651, n = 2) | 123 ± 4.24 |
| 54 | 76.8 | (8.87, n = 2) | 137 ± 14.1 |
| 55 | 49.1 | (4.24, n = 2) | 109 ± 0.179 |
| 56 | 84.6 | (10.1, n = 3) | 113 ± 10.7 |
| 57 | 61.0 | (6.27, n = 3) | 112 ± 14.8 |
| 58 | 23.8 | (1.35, n = 2) | 110 ± 1.71 |
| 59 | 49.1 | (1.86, n = 2) | 122 ± 2.6 |
| 60 | 189 | (26.9, n = 2) | 89.8 ± 3.63 |
| 61 | 56.8 | (21.5, n = 2) | 88.5 ± 2.66 |
| 62 | 17.5 | (1.42, n = 2) | 67.8 ± 10.2 |
| 63 | 21.6 | (4.35, n = 7) | 62.1 ± 3.55 |
| 64 | 61.9 | | 128 |
| 65 | 38.1 | (4.89, n = 2) | 113 ± 4.23 |
| 66 | 99.3 | (3.01, n = 2) | 118 ± 0.533 |
| 67 | 45.0 | (0.00697, n = 2) | 115 ± 8.68 |
| 68 | 0.904 | (0.212, n = 3) | 86.9 ± 7.93 |
| 69 | 34.0 | (2.62, n = 4) | 87.8 ± 8.06 |
| 70 | 158 | (19.1, n = 4) | 102 ± 2.2 |
| 71 | 74.8 | (1.44, n = 2) | 88.4 ± 10.3 |
| 72 | 300 | (62.0, n = 2) | 102 ± 4.83 |
| 73 | 11.1 | (0.396, n = 2) | 91.6 ± 0.83 |
| 74 | 13.6 | (2.34, n = 3) | 102 ± 3.65 |
| 75 | 65.0 | (17.5, n = 3) | 119 ± 6.64 |
| 76 | 16.2 | (0.585, n = 3) | 97.2 ± 7.63 |
| 77 | 88.2 | (8.82, n = 2) | 103 ± 12 |
| 78 | 20.3 | (2.18, n = 2) | 113 ± 18.8 |
| 79 | 25.1 | (2.88, n = 2) | 104 ± 5 |
| 80 | 28.6 | (0.471, n = 3) | 109 ± 4.1 |
| 81 | 33.1 | (7.90, n = 2) | 117 ± 0.667 |
| 82 | 36.4 | (8.35, n = 3) | 106 ± 2.58 |
| 83 | 44.1 | (4.69, n = 3) | 95.4 ± 7.12 |
| 84 | 37.8 | | 89.7 |
| 85 | 41.0 | (7.12, n = 2) | 87 ± 0.761 |
| 86 | 10.2 | (0.895, n = 2) | 71.1 ± 2.32 |
| 87 | 52.7 | (7.65, n = 2) | 71.1 ± 9.09 |
| 88 | 89 | | 116 |
| 89 | 152 | | 32.3 |
| 90 | 45.5 | | 120 |
| 91 | 60.9 | | 114 |
| 92 | 47.1 | (2.70, n = 7) | 126 ± 1.93 |
| 93 | 15.3 | (2.33, n = 2) | 115 ± 1.41 |
| 94 | 35.4 | | 101 |
| 95 | 48.1 | (15.0, n = 2) | 90 ± 2.84 |
| 96 | 169 | (38.6, n = 2) | 104 ± 4.57 |
| 97 | 38.6 | (7.28, n = 6) | 96.6 ± 4.57 |
| 98 | 35.5 | (8.28, n = 2) | 110 ± 3.22 |
| 99 | 6.84 | (0.274, n = 2) | 69.2 ± 0.431 |
| 100 | 3.47 | (0.582, n = 4) | 70.9 ± 1.67 |
| 101 | 58.5 | (9.71, n = 2) | 59.6 ± 0.0693 |
| 102 | 50.9 | (18.2, n = 2) | 69.4 ± 0.0734 |
| 103 | 99.6 | (17.0, n = 2) | 73.3 ± 6.2 |
| 104 | 54.6 | (1.64, n = 2) | 89 ± 1.12 |
| 105 | 49.8 | (13.0, n = 2) | 8.43 ± 1.23 |
| 106 | 36.3 | (0.216, n = 2) | 77.3 ± 7.69 |
| 107 | 36.2 | (9.99, n = 2) | 108 ± 0.709 |
| 108 | 0.470 | (0.0482, n = 3) | 81.7 ± 1.63 |
| 109 | 0.701 | (0.107, n = 2) | 73.3 ± 2.59 |
| 110 | 4.34 | (0.409, n = 4) | 50.8 ± 1.54 |
| 111 | 6.75 | (0.393, n = 3) | 28 ± 0.197 |
| 112 | 3.21 | (0.176, n = 4) | 62 ± 0.9 |
| 113 | 1.22 | (0.0873, n = 4) | 45.5 ± 2.18 |
| 114 | 1.79 | (0.0653, n = 2) | 80.3 ± 0.191 |
| 115 | 4.65 | (0.439, n = 2) | 87.3 ± 0.526 |
| 116 | 127 | (4.28, n = 2) | 23.1 ± 0.52 |
| 117 | 4.39 | (0.341, n = 4) | 56 ± 3.91 |
| 118 | 4.02 | (0.130, n = 2) | 59.3 ± 1.52 |
| 119 | 144 | (11.6, n = 2) | 79.5 ± 5.3 |
| 120 | 33.5 | (3.77, n = 2) | 72.4 ± 2.16 |
| 121 | 107 | (1.34, n = 2) | 74.6 ± 1.47 |
| 122 | 103 | (0.656, n = 2) | 90.8 ± 7.25 |
| 123 | 18.7 | (0.0410, n = 2) | 95.7 ± 6.9 |
| 124 | 55.6 | (6.04, n = 2) | 56.9 ± 11.2 |
| 125 | 38.5 | (8.70, n = 2) | 68.5 ± 6.59 |
| 126 | 42.3 | (9.23, n = 2) | 93.6 ± 1.06 |
| 127 | 60.1 | (20.2, n = 2) | 56.1 ± 4.15 |
| 128 | 163 | (61.7, n = 2) | 85.2 ± 1.24 |
| 129 | 138 | (22.6, n = 2) | 78.6 ± 6.28 |
| 130 | 104 | (18.0, n = 2) | 74.9 ± 11.6 |
| 131 | 17.7 | (2.20, n = 2) | 57.9 ± 4.24 |
| 132 | 20.3 | (1.51, n = 2) | 93.3 ± 0.0968 |
| 133 | 13.7 | (3.80, n = 2) | 78.4 ± 3.18 |
| 134 | 7.63 | (0.401, n = 2) | 57.4 ± 0.103 |
| 135 | 5.61 | (0.659, n = 2) | 48.5 ± 2.32 |
| 136 | 10.8 | (0.359, n = 2) | 74.6 ± 6.02 |
| 137 | 12.3 | (1.36, n = 2) | 61.7 ± 1.34 |
| 138 | 99.2 | | 52.3 |
| 139 | 24.8 | (4.37, n = 2) | 99.7 ± 3.49 |
| 140 | 51.7 | (2.85, n = 2) | 84.8 ± 5.99 |
| 141 | 26.2 | (2.86, n = 2) | 60.9 ± 5.88 |
| 142 | 102 | (15.8, n = 2) | 49.9 ± 1.38 |
| 143 | 57.7 | (0.677, n = 2) | 72.8 ± 3.32 |
| 144 | 0.755 | (0.0579, n = 4) | 63.3 ± 2.24 |
| 145 | 0.727 | (0.0589, n = 3) | 96.6 ± 2.37 |
| 146 | 0.234 | (0.0161, n = 2) | 120 ± 5.11 |
| 147 | 17.2 | (2.46, n = 3) | 86.1 ± 9 |
| 148 | 1.13 | (0.0816, n = 2) | 74.4 ± 2.94 |
| 149 | 5.57 | (0.171, n = 3) | 73.2 ± 2.43 |
| 150 | 2.06 | (0.376, n = 3) | 89.5 ± 6.21 |
| 151 | 16.9 | (0.0719, n = 2) | 106 ± 2.17 |
| 152 | 1.73 | (0.0497, n = 3) | 87.2 ± 1.03 |
| 153 | 0.478 | (0.0734, n = 3) | 77 ± 2.05 |
| 154 | 5.24 | (1.02, n = 2) | 74.2 ± 2.83 |
| 155 | 118 | (0.880, n = 2) | 78.1 ± 4.92 |
| 156 | 27.2 | (4.74, n = 2) | 34.8 ± 1.74 |
| 157 | 108 | (2.63, n = 2) | 16.5 ± 0.234 |
| 158 | 1.23 | (0.0650, n = 5) | 102 ± 1.41 |
| 159 | 0.397 | (0.0334, n = 7) | 95.3 ± 3.03 |
| 160 | 1.24 | (0.0489, n = 2) | 119 ± 3.76 |
| 161 | 1.47 | (0.0589, n = 2) | 71.7 ± 1.02 |
| 162 | 0.179 | | 50 |
| 163 | 3.95 | | 109 |
| 164 | 4.31 | (0.942, n = 2) | 53.3 ± 0.286 |
| 165 | 0.440 | (0.0392, n = 3) | 65.7 ± 4.25 |
| 166 | 0.359 | (0.0144, n = 2) | 116 ± 2.13 |
| 167 | 0.753 | (0.114, n = 3) | 92.1 ± 0.883 |
| 168 | 1.81 | | 110 |
| 169 | 1.15 | (0.134, n = 4) | 58.9 ± 1.86 |
| 170 | 0.846 | (0.0684, n = 4) | 97.2 ± 0.998 |
| 171 | 14.2 | (1.40, n = 2) | 25.1 ± 2.38 |
| 172 | 3.16 | (0.0694, n = 2) | 12.6 ± 0.102 |
| 173 | 24.6 | (4.97, n = 2) | 15.9 ± 0.138 |
| 174 | 6.09 | (0.352, n = 2) | 13.4 ± 1.64 |
| 175 | 0.996 | (0.154, n = 2) | 112 ± 1.44 |
| 176 | 14.4 | | 123 |
| 177 | 0.845 | (0.376, n = 5) | 115 ± 1.59 |
| 178 | 0.743 | (0.0881, n = 3) | 39.9 ± 0.556 |
| 179 | 2.95 | (0.439, n = 2) | 75.9 ± 4.59 |
| 180 | 7.75 | (0.440, n = 4) | 35.4 ± 1.96 |
| 181 | 23.1 | (1.81, n = 2) | 25.5 ± 2.92 |
| 182 | 41.4 | (3.89, n = 2) | 30.4 ± 2.08 |
| 183 | 44.9 | (1.18, n = 2) | 4.64 ± 0.608 |
| 184 | 15.2 | (0.727, n = 2) | 15.2 ± 1.27 |
| 185 | 0.402 | (0.00717, n = 2) | 115 ± 5.3 |
| 186 | 1.75 | (0.337, n = 2) | 104 ± 0.548 |
| 187 | 1.26 | (0.0235, n = 2) | 84.6 ± 2.33 |
| 188 | 0.265 | (0.0102, n = 2) | 110 ± 4.51 |
| 189 | 1.30 | (0.0226, n = 2) | 61 ± 0.6 |
| 190 | 1.12 | (0.101, n = 2) | 80.2 ± 6.78 |
| 191 | 2.89 | (0.0500, n = 2) | 78.5 ± 0.865 |
| 192 | 0.0409 | | 129 |
| 193 | 1.33 | (0.211, n = 2) | 85.5 ± 0.117 |
| 194 | 0.567 | (0.0884, n = 2) | 64.1 ± 0.489 |

| | | |
|---|---|---|
| 195 | 2.36 | 88.8 |
| 196 | 0.601 | 95.7 |
| 197 | 0.660 | 119 |
| 198 | 1.81 | 90.3 |

^{a} EC₅₀, nM = the Geometric Mean with the Standard Error of the Mean followed by the number of observations in parenthesis.
^{b} Eₘₐₓ, % = the Arithmetic Mean ± the Standard Error of the Mean for the percent of maximal response to GIP(1-42)OH.

We claim:

1. A compound of the formula: 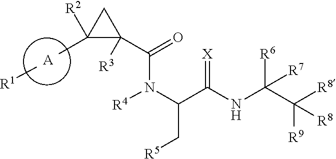
wherein
R¹ is:
i) an 8-, 9- or 10-membered N-containing bicyclic heterocycle optionally substituted with 1 to 3 substituents independently selected from:
C₃-C₄cycloalkyl optionally substituted with 1 or 2 halo or CH₃,
4-membered heterocycle optionally substituted with CH₃,
C₁-C₄alkoxy,
C₁-C₄haloalkyl,
C₁-C₄haloalkoxy,
oxo,
CN,
halo,
CD₃, and
C₁-C₄alkyl optionally substituted with OCH₃, OH or C₃-C₄cycloalkyl, wherein the C₃-C₄cycloalkyl is optionally substituted with 1 or 2 halo,
ii) NR¹⁶COR¹²,
iii) OH, or
iv) a 5- or 6-membered heterocycle optionally substituted with 1 or 2 substituents independently selected from: C(O)CH₃, oxo, OCH₃, CN, halo, and C₁-C₄alkyl optionally substituted with OH;
R¹² is NHR¹³, pyridyl, phenyl, OCH₃ or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
R¹⁶ is H or CH₃;
A is phenyl, a 6-membered N-containing heteroaryl or thienyl, wherein the phenyl, heteroaryl or thienyl is optionally substituted with 1 or 2 substituents independently selected from halo, CF₃, CHF₂, CH₃, cyclopropyl, OH and CN, or when R¹ is an optionally substituted 8-, 9- or 10-membered N-containing bicyclic heterocycle A may be absent;
R² is H, CH₃, halo, CF₃ or CHF₂;
R³ is H and R⁴ is H, CH₃ or CD₃; or R³ and R⁴ together form —CH₂—CH₂—;
R⁵ is:
i) a 8- or 9-membered N-containing bicyclic heterocycle optionally substituted with 1 or 2 substituents selected from oxo, halo and CH₃, or
ii) a 5- or 6-membered N-containing heteroaryl optionally substituted with 1 or 2 substituents selected from CH₃, CD₃, CHF₂, OH and CH₂CN:
X is O or S;
R⁶ and R⁷ are each CH₃, or together form a cyclopropyl, cyclobutyl, oxetane, tetrahydrofuran, pyrrolidine or piperidine, wherein the cyclopropyl or cyclobutyl is optionally substituted with 1 or 2 halo and wherein the pyrrolidine or piperidine is optionally substituted with CH₃;
R⁸ is H, D, OH, CO₂R¹⁸, or 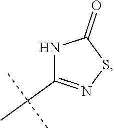
wherein R¹⁸ is H or C₁-C₄alkyl optionally substituted with OC(O)C₁-C₄alkyl or morpholine, R^{8′} is H, and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ is D, R^{8′} is D, and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo, and R^{8′} is H;
R¹⁰ is CF₃, CF₂H, halo or cyclopropyl;
R¹¹ is:
i) a 5- or 6-membered heterocycle optionally substituted with 1 to 4 substituents independently selected from:
halo,
OH,
C₁-C₄alkoxy,
CD₃,
OCHF₂,
SO₂NH₂,
CN,
C₁-C₄haloalkyl,
C₃-C₆cycloalkyl,
C(O)NH₂,
a 4- to 6-membered heterocycle optionally substituted with oxo or CH₃,
oxo,
CR¹⁴R¹⁵OP(O)(OH)₂,
(CH₂)ₘP(O)(R¹⁷) 2, and
C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
ii) a 9- or 10-membered N-containing bicyclic heterocycle optionally substituted with 1 or 2 substituents independently selected from CN, CH₃, halo, oxo, C(O)O(CH₃)₃ and OH,
iii) C₁-C₄alkoxy,
iv) C₃-C₅cycloalkyl,
v) halo,
vi) (CH₂)ₙC(O)R¹⁹, wherein n is 0 or 1, R¹⁹ is OCH₃, NR¹⁴R¹⁵ or a 4- to 6-membered heterocycle,
vii) (CH₂)ₘP(O)(R¹⁷)₂,
viii) C₁-C₄alkyl optionally substituted with a 4- to 6-membered heterocycle or phenyl, which heterocycle or phenyl is optionally substituted by CH₃,
ix) CN, or
x)phenyl optionally substituted with 1 to 3 substituents independently selected from: halo, C₁-C₄alkyl, (CH₂)ₘP(O)(R¹⁷)₂, COOH and NR¹⁴R¹⁵, wherein the C₁-C₄alkyl is optionally substituted with OH; and
R¹⁴ and R¹⁵ are independently H or CH₃;
each R¹⁷ is independently C₁-C₄alkyl; and
m is 0 or 1;
or a pharmaceutically acceptable salt thereof.

2. The compound according to claim 1, wherein X is O, or a pharmaceutically acceptable salt thereof.

3. The compound according to claim 1, wherein R⁶ and R⁷ are each CH₃, or a pharmaceutically acceptable salt thereof.

4. The compound according to claim 1, wherein R³ is H and R⁴ is H, CH₃ or CD₃, or a pharmaceutically acceptable salt thereof.

5. The compound according to claim 1, wherein R³ and R⁴ together form —CH₂—CH₂—, or a pharmaceutically acceptable salt thereof.

6. The compound according to claim 1, wherein R² is H or CH₃, or a pharmaceutically acceptable salt thereof.

7. The compound according to claim 1, wherein R¹ is:
i) a 9- or 10-membered N-containing bicyclic heterocycle selected from: 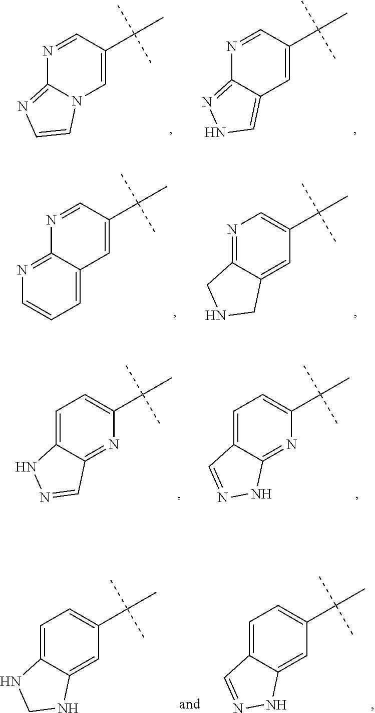
optionally substituted with 1 to 3 substituents independently selected from:
C₃-C₄cycloalkyl optionally substituted with 1 or 2 halo or CH₃,
4-membered heterocycle optionally substituted with CH₃,
C₁-C₄alkoxy,
C₁-C₄haloalkyl,
oxo,
CN,
halo, and
C₁-C₄alkyl optionally substituted with OCH₃, OH or C₃-C₄cycloalkyl, wherein the C₃-C₄cycloalkyl is optionally substituted with 1 or 2 halo,
ii) NR¹⁶COR¹², wherein R¹² is NHR¹³, OCH₃ or C₃-C₄cycloalkyl optionally substituted with 1 or 2 halo, R¹³ is phenyl optionally substituted with 1 or 2 halo and R¹⁶ is H or CH₃, or
iii) piperazine optionally substituted with C(O)CH₃,
pyridine optionally substituted with 1 or 2 substituents independently selected from OCH₃, halo and C₁-C₄alkyl optionally substituted with OH, or
pyrimidine optionally substituted with 1 or 2 substituents independently selected from OCH₃, CH₃ and oxo, or a pharmaceutically acceptable salt thereof.

8. The compound according to claim 7, wherein R¹ is selected from: 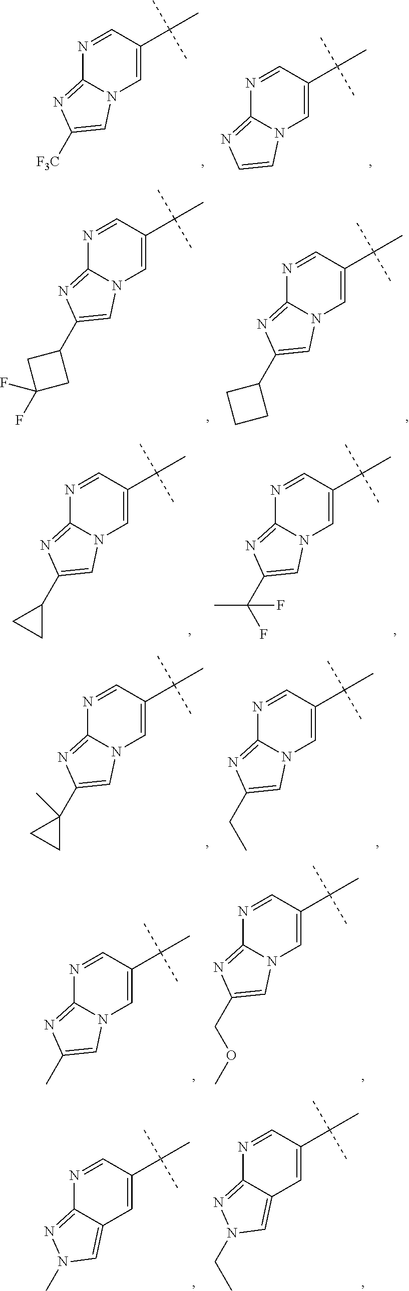 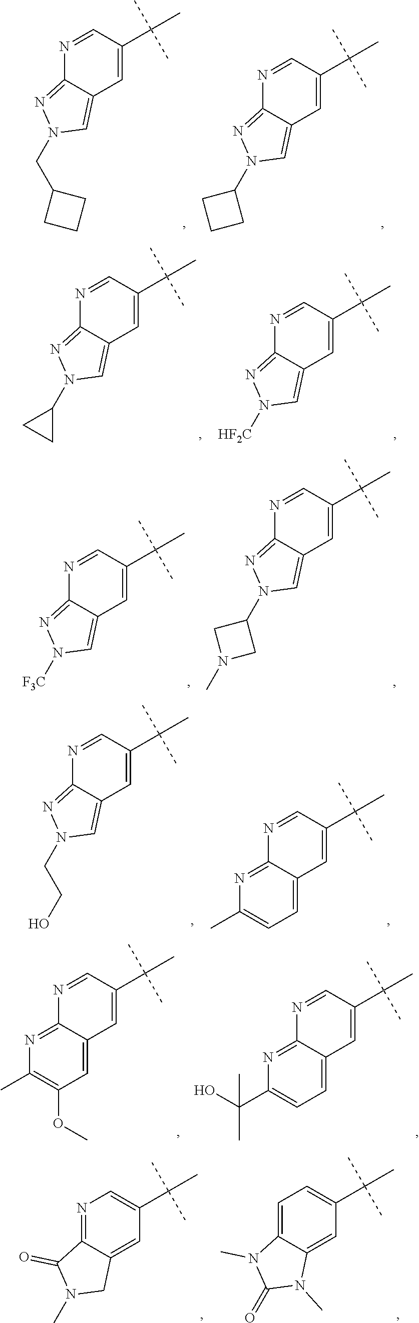 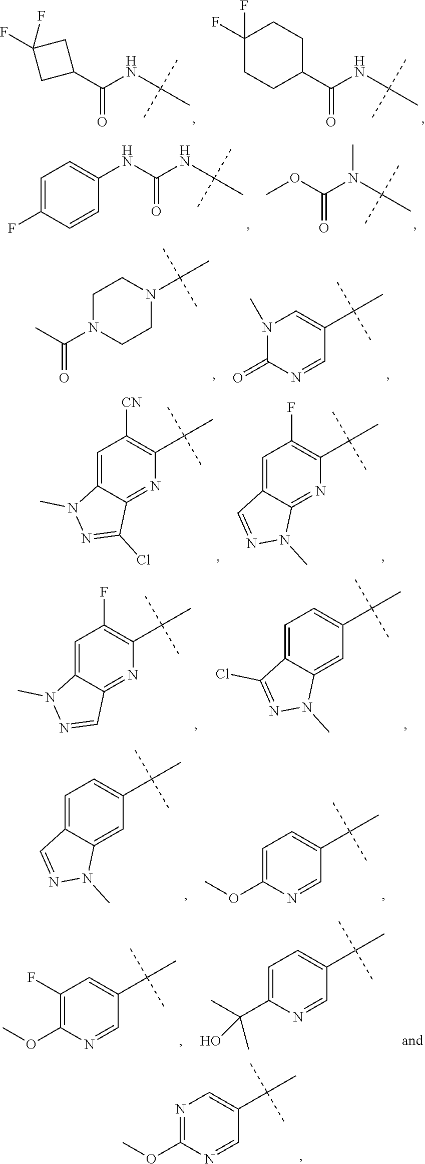
or a pharmaceutically acceptable salt thereof.

9. The compound according to claim 8, wherein R¹ is selected from: 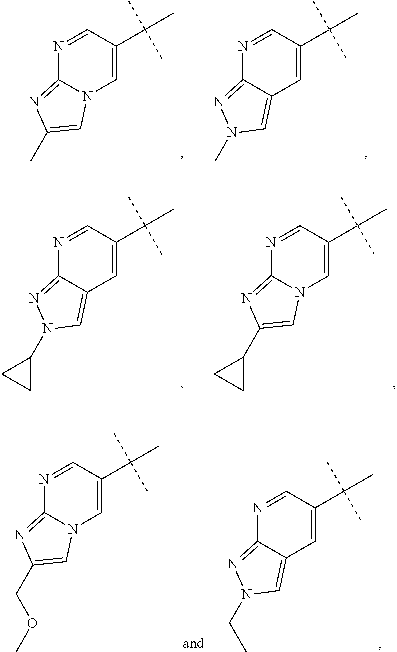
or a pharmaceutically acceptable salt thereof.

10. The compound according to claim 1, wherein A is phenyl, pyridyl, pyrimidinyl or thienyl, wherein the phenyl, pyridyl, pyrimidinyl or thienyl is optionally substituted with 1 or 2 substituents independently selected from: F, Cl, CF₃, CHF₂, CH₃, cyclopropyl and OH, or when R¹ is an optionally substituted 8-, 9- or 10-membered N-containing bicyclic heterocycle A may be absent, or a pharmaceutically acceptable salt thereof.

11. The compound according to claim 10, wherein A is selected from: 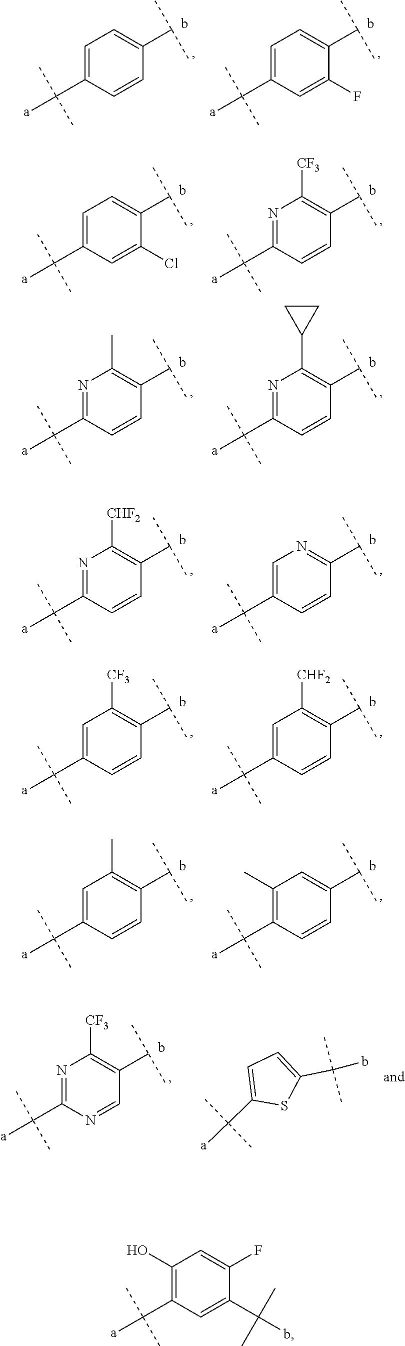
wherein a is the point of attachment to R¹ and b is the point of attachment to the cyclopropyl, or A is absent, or a pharmaceutically acceptable salt thereof.

12. The compound according to claim 11, wherein A is selected from: 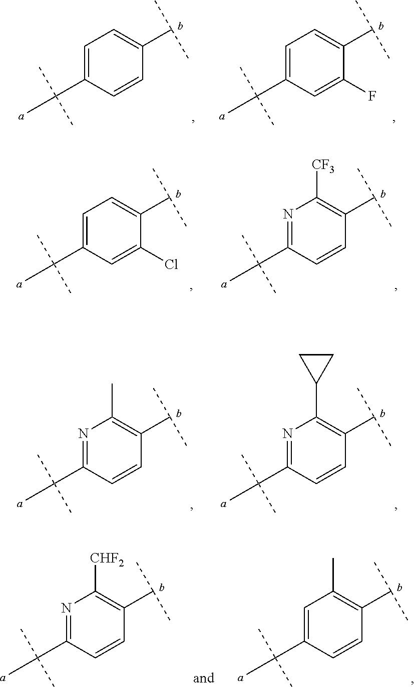
or a pharmaceutically acceptable salt thereof.

13. The compound according to claim 1, wherein R⁵ is:
i) a 9-membered N-containing bicyclic heterocycle, or
ii) a 5-membered N-containing heteroaryl substituted with CH₃, CD₃ or CHF₂,
or a pharmaceutically acceptable salt thereof.

14. The compound according to claim 13, wherein R⁵ is: 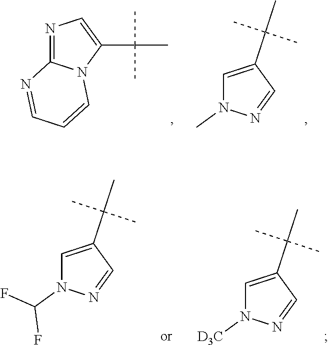
or a pharmaceutically acceptable salt thereof.

15. The compound according to claim 14, wherein R⁵ is: 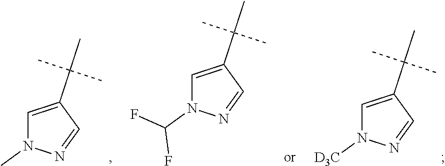
or a pharmaceutically acceptable salt thereof.

16. The compound according to claim 1, wherein R⁸ and R^{8′} are H, or a pharmaceutically acceptable salt thereof.

17. The compound according to claim 1, wherein R⁹ is:
phenyl substituted with F, or
a 5- or 6-membered N-containing heteroaryl optionally substituted with R¹⁰ or with R¹⁰ and R¹¹, or a pharmaceutically acceptable salt thereof.

18. The compound according to claim 17, wherein R⁹ is: 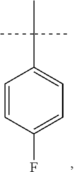
or
a 5- or 6-membered N-containing heteroaryl selected from: 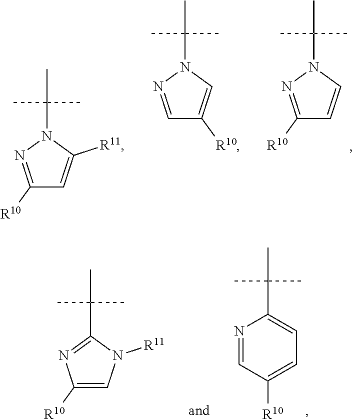
or a pharmaceutically acceptable salt thereof.

19. The compound according to claim 18, wherein R⁹ is:
a 5- or 6-membered N-containing heteroaryl selected from: 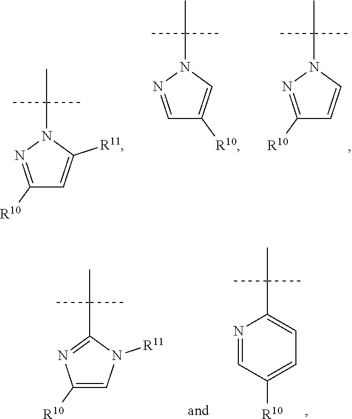
R¹⁰ is CF₃, CF₂H or F, and R¹¹ is:
i) a 5- or 6-membered heterocycle optionally substituted with 1 to 4 substituents independently selected from:
halo,
OH,
CD₃,
C₁-C₄haloalkoxy,
CN,
C₁-C₄haloalkyl,
C₃-C₅cycloalkyl,
C(O)NH₂,
a 4- to 6-membered heterocycle optionally substituted with oxo,
oxo,
CR¹⁴R¹⁵OP(O)(OH)₂, and
C₁-C₄alkyl, wherein said C₁-C₄ alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
ii) a 9- or 10-membered N-containing bicyclic heterocycle optionally substituted with 1 to 2 substituents independently selected from CH₃, halo, oxo and C(O)O(CH₃)₃,
iii) C₃-C₅cycloalkyl,
iv) halo,
v) C(O)OCH₃,
vi) P(O)(CH₃)₂, or
vii)phenyl optionally substituted with 1 to 3 substituents independently selected from: halo, P(O)(CH₃)₂, and C₁-C₄alkyl, wherein the C₁-C₄alkyl is optionally substituted with OH;
R¹⁴ and R¹⁵ are independently H or CH₃, or a pharmaceutically acceptable salt thereof.

20. The compound according to claim 19, wherein R⁹ is: 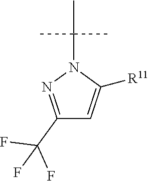
and R¹¹ is:
i) a 5- or 6-membered heterocycle selected from: 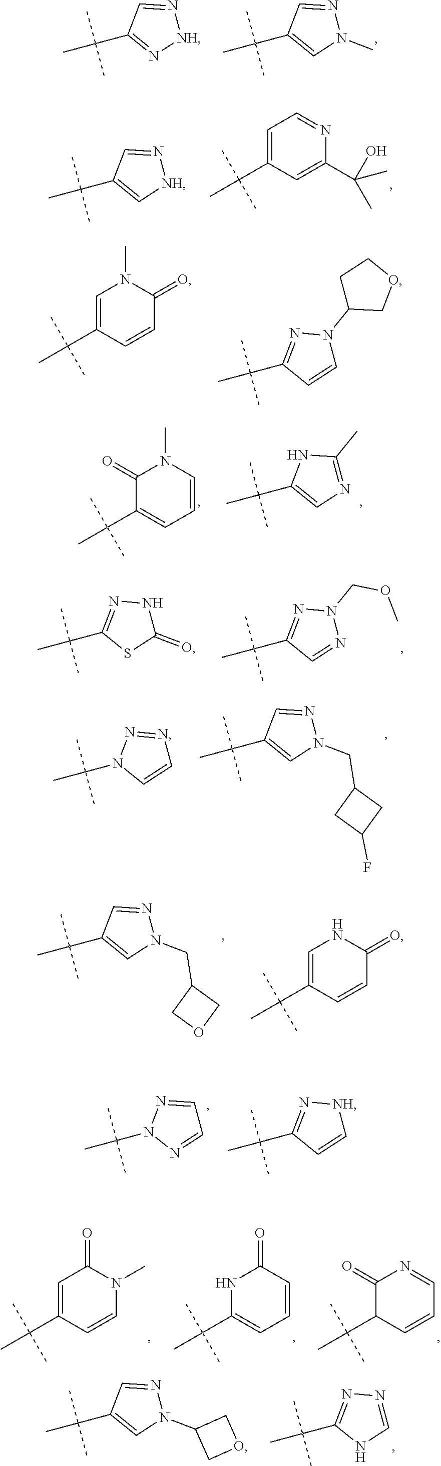 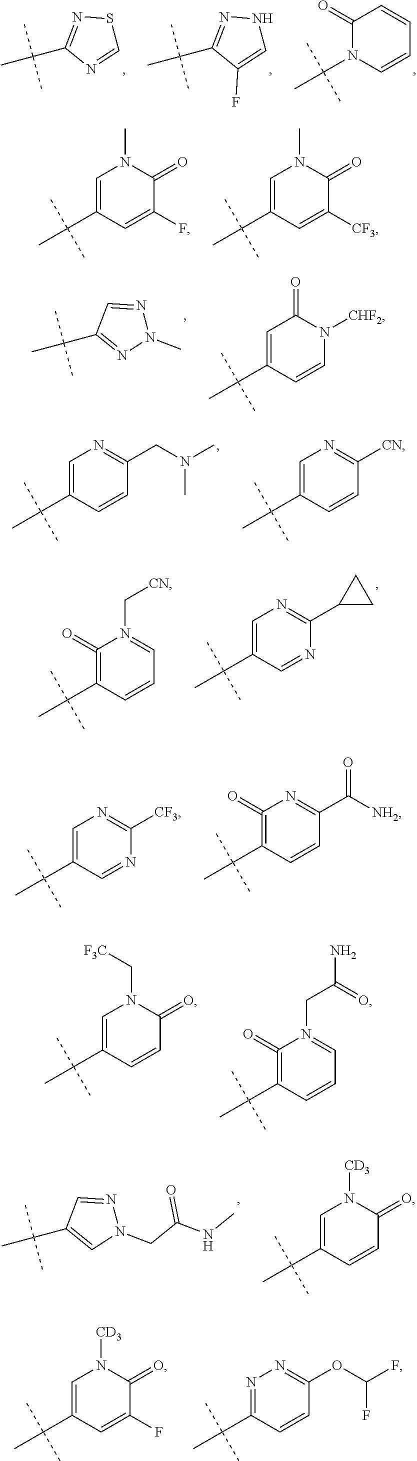 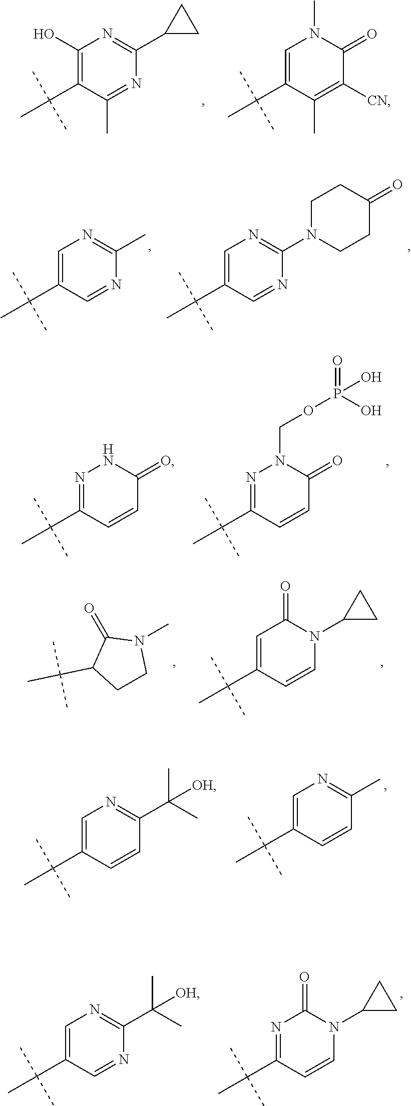 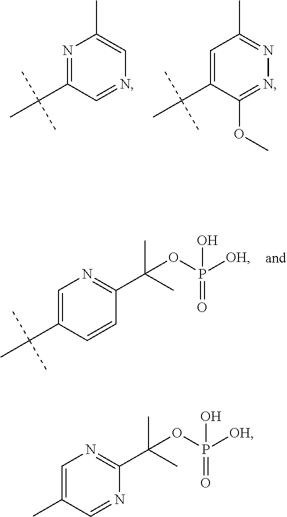
ii) a 9- or 10-membered N-containing bicyclic heterocycle selected from: 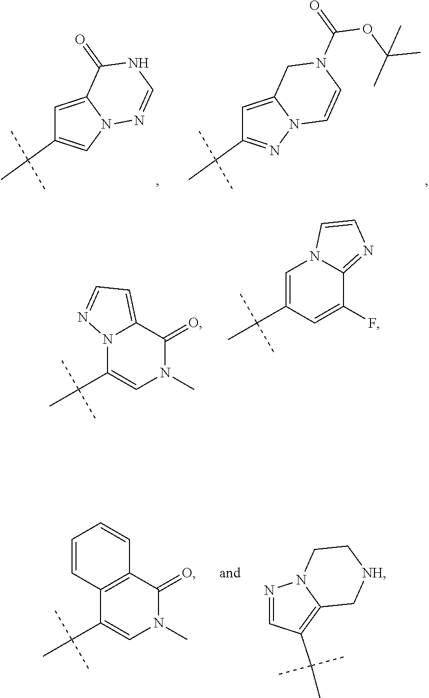
iii) cyclopropyl, cyclobutyl,
iv) Br,
v) C(O)OCH₃,
vi) P(OX(CH₃)₂, or 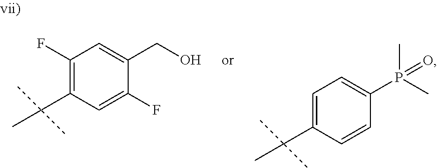
or a pharmaceutically acceptable salt thereof.

21. The compound according to claim 20, wherein R¹¹ is 5- or 6-membered heterocycle selected from: 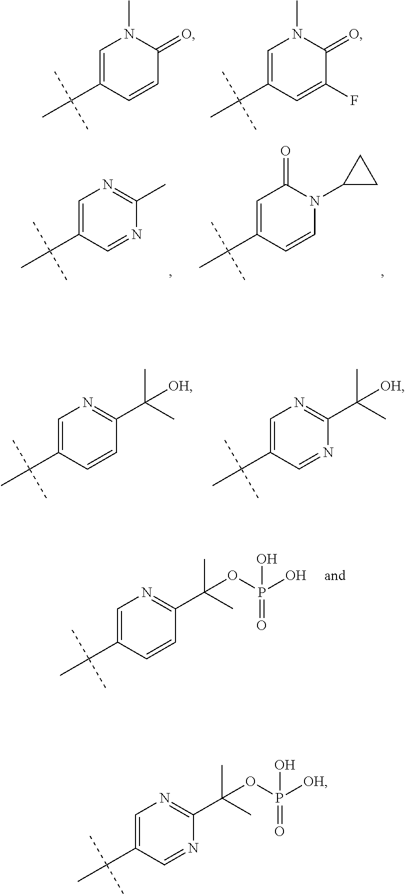
or a pharmaceutically acceptable salt thereof.

22. The compound according to claim 1 of the formula: 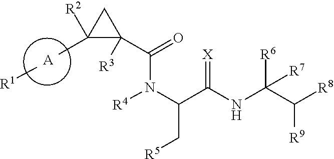
wherein
R¹ is:
i) an 8-, 9- or 10-membered N-containing bicyclic heterocycle optionally substituted with 1 to 3 substituents independently selected from:
C₃-C₄cycloalkyl optionally substituted with 1 or 2 halo or CH₃,
4-membered heterocycle optionally substituted with CH₃,
C₁-C₄alkoxy,
C₁-C₄haloalkyl,
oxo,
CN,
halo, and
C₁-C₄alkyl optionally substituted with OCH₃, OH or C₃-C₄cycloalkyl, wherein the C₃-C₄cycloalkyl is optionally substituted with 1 or 2 halo,
ii) NHCOR¹²,
iii) OH, or
iv) a 5- or 6-membered heterocycle optionally substituted with 1 or 2 substituents independently selected from: C(O)CH₃, C₁-C₄alkyl, oxo and CN;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
A is phenyl, a 6-membered N-containing heteroaryl or thienyl, wherein the phenyl, heteroaryl or thienyl is optionally substituted with 1 or 2 substituents independently selected from halo, CF₃, CHF₂, CH₃, cyclopropyl or CN, or when R¹ is an optionally substituted 8-, 9- or 10-membered N-containing bicyclic heterocycle A may be absent;
R² is H, CH₃, halo, CF₃ or CHF₂;
R³ is H and R⁴ is H, CH₃ or CD₃; or R³ and R⁴ together form —CH₂—CH₂—;
R⁵ is:
i) a 8- or 9-membered N-containing bicyclic heterocycle optionally substituted with halo, or
ii) a 5- or 6-membered N-containing heteroaryl optionally substituted with CH₃, CD₃, CHF₂ or OH;
X is O or S;
R⁶ and R⁷ are each CH₃ or together form a cyclopropyl;
R⁸ is H, CH₃, CO₂H or C(O)OCH₃ and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from:
halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
i) a 5- or 6-membered heterocycle optionally substituted with 1 to 4 substituents independently selected from:
halo,
OH,
OCH₃,
CD₃,
OCHF₂,
SO₂NH₂,
CN,
C₁-C₄haloalkyl,
C₃-Cscycloalkyl,
C(O)NH₂,
a 4- to 6-membered heterocycle optionally substituted with oxo,
oxo,
CR¹⁴R¹⁵OP(O)(OH)₂, and
C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
ii) a 9- or 10-membered N-containing bicyclic heterocycle optionally substituted with 1 or 2 substituents independently selected from CN, CH₃, halo, oxo, C(O)O(CH₃)₃ and OH,
iii) C₁-C₄alkoxy,
iv) C₃-C₅cycloalkyl,
v) halo,
vi) C(O)OCH₃, or
vii)phenyl optionally substituted with 1 to 3 substituents independently selected from: halo and C₁-C₄alkyl, wherein the C₁-C₄alkyl is optionally substituted with OH; and
R¹⁴ and R¹⁵ are independently H or CH₃;
or a pharmaceutically acceptable salt thereof.

23. The compound of claim 1, wherein
R¹ is:
i) a 9- or 10-membered N-containing bicyclic heterocycle optionally substituted with 1 or 2 substituents independently selected from:
C₃-C₄cycloalkyl optionally substituted with 1 or 2 halo or CH₃,
4-membered heterocycle optionally substituted with CH₃,
C₁-C₄alkoxy,
C₁-C₄haloalkyl,
oxo, and
C₁-C₄alkyl optionally substituted with OCH₃, OH or C₃-C₄cycloalkyl, wherein the C₃-C₄cycloalkyl is optionally substituted with 1 or 2 halo,
ii) NHCOR¹²,
iii) OH, or
iv) a 6-membered heterocycle optionally substituted with 1 or 2 substituents independently selected from: C(O)CH₃, C₁-C₄alkyl and oxo;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
A is phenyl, a 6-membered N-containing heteroaryl or thienyl, wherein the phenyl, heteroaryl or thienyl is optionally substituted with 1 or 2 substituents independently selected from halo, CF₃, CHF₂, CH₃ and cyclopropyl;
R² is H, CH₃, halo, CF₃ or CHF₂;
R³ is H and R⁴ is H, CH₃ or CD₃; or R³ and R⁴ together form —CH₂—CH₂—;
R⁵ is:
i) a 8- or 9-membered N-containing bicyclic heterocycle optionally substituted with halo, or
ii) a 5- or 6-membered N-containing heteroaryl optionally substituted with CH₃, CD₃, CHF₂ or OH;
X is O or S;
R⁶ and R⁷ are each CH₃ or together form a cyclopropyl;
R⁸ is H, CH₃, CO₂H or C(O)OCH₃ and R⁹ is a 5- or 6-membered N-containing heteroaryl or phenyl, wherein the heteroaryl or phenyl is optionally substituted with R¹⁰ or with R¹⁰ and R¹¹, or wherein the phenyl is optionally substituted with 1 or 2 substituents selected from:
halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
i) a 5- or 6-membered heterocycle optionally substituted with 1 to 4 substituents independently selected from:
halo,
OH,
OCH₃,
CD₃,
OCHF₂,
SO₂NH₂,
CN,
C₁-C₄haloalkyl,
C₃-C₅cycloalkyl,
C(O)NH₂,
a 4- to 6-membered heterocycle optionally substituted with oxo,
oxo, and
C₁-C₄alkyl, wherein the C₁-C₄alkyl is optionally substituted with OH, OCH₃, C(O)NR¹⁴R¹⁵, CN, NR¹⁴R¹⁵, a 4- to 6-membered heterocycle or C₃-C₄cycloalkyl optionally substituted with halo,
ii) a 9- or 10-membered N-containing bicyclic heterocycle optionally substituted with 1 or 2 substituents independently selected from CN, CH₃, halo, oxo, C(O)O(CH₃)₃ and OH,
iii) C₁-C₄alkoxy,
iv) C₃-C₅cycloalkyl,
v) halo,
vi) C(O)OCH₃, or
vii)phenyl optionally substituted with 1 to 3 substituents independently selected from: halo and C₁-C₄alkyl, wherein the C₁-C₄alkyl is optionally substituted with OH; and
R¹⁴ and R¹⁵ are independently H or CH₃;
or a pharmaceutically acceptable salt thereof.

24. The compound according to claim 1 of the formula: 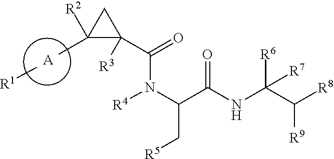
wherein
R¹ is:
i) a 9- or 10-membered N-containing bicyclic heterocycle optionally substituted with 1 or 2 substituents independently selected from:
C₃-C₄cycloalkyl optionally substituted with 1 or 2 halo or CH₃,
C₁-C₄haloalkyl,
oxo, and
C₁-C₄alkyl optionally substituted with OCH₃ or C₃-C₄cycloalkyl, wherein the C₃-C₄cycloalkyl is optionally substituted with 1 or 2 halo,
ii) NHCOR¹², or
iii) OH;
R¹² is NHR¹³, pyridyl, phenyl or C₃-C₆cycloalkyl, wherein the pyridyl, phenyl or C₃-C₆cycloalkyl is optionally substituted with 1 or 2 halo;
R¹³ is C₁-C₄alkyl, or phenyl optionally substituted with 1 or 2 halo;
R² is H, CH₃, halo, CF₃ or CHF₂;
R³ is H and R⁴ is H or CH₃; or R³ and R⁴ together form —CH₂—CH₂—;
R⁵ is:
i) a 8- or 9-membered N-containing bicyclic heterocycle optionally substituted with halo, or
ii) a 5- or 6-membered N-containing heteroaryl optionally substituted with CH₃, CHF₂ or OH;
R⁶ and R⁷ are each CH₃ or together form a cyclopropyl;
R⁸ is H, CH₃, CO₂H or C(O)OCH₃ and R⁹ is:
a 5- or 6-membered N-containing heteroaryl optionally substituted with R¹⁰ or with R¹⁰ and R¹¹, or
phenyl optionally substituted with 1 or 2 substituents selected from: halo, CF₃ and pyrazine optionally substituted with CF₂H or OCH₃;
or R⁸ and R⁹ together form a 9-membered N-containing bicyclic heterocycle optionally substituted with 1 to 3 substituents independently selected from: CH₃, CF₃ and oxo;
R¹⁰ is CF₃, CF₂H or halo;
R¹¹ is:
i) a 5- or 6-membered heterocycle optionally substituted with 1 to 3 substituents independently selected from:
halo,
OH,
OCH₃,
SO₂NH₂,
a 4- to 6-membered heterocycle,
oxo or
C₁-C₄alkyl, wherein the C₁-C₄alkyl is optionally substituted with OH, OCH₃, a 4- to 6-membered heterocycle or C₃-C₄cycloalkyl optionally substituted with halo,
ii) a 9-membered N-containing bicyclic heterocycle optionally substituted with CN, oxo, C(O)O(CH₃)₃ or OH,
iii) C₁-C₄alkoxy,
iv) C₃-C₅cycloalkyl,
v) halo, or
vi) C(O)OCH₃;
or a pharmaceutically acceptable salt thereof.

25. The compound according to claim 1, wherein the compound is selected from Examples 11 to 198, a pharmaceutically acceptable salt thereof.

26. A pharmaceutical composition comprising a compound, or a pharmaceutically acceptable salt thereof, according to claim 1 and at least one pharmaceutically acceptable carrier, diluent, or excipient.

27. A method for treating type II diabetes mellitus comprising administering to a patient in need thereof a therapeutically effective amount of a compound according to claim 1, or a pharmaceutically acceptable salt thereof.

28. A method for treating obesity comprising administering to a patient in need thereof a therapeutically effective amount of a compound according to claim 1, or a pharmaceutically acceptable salt thereof.

29. (canceled)

30. (canceled)

31. (canceled)
